SEQUENCE LISTING (783 sequences)

SEQ ID NO: 1 - DNA, 4302 bp, Artificial Sequence
  Synthetic
atggcggcgggctcggatctgctggacgaggtcttcttcaacagcgaggtggacgagaaagtggtgagcgacctggtgggctcgctggagtcgcagctggcggccagcgcggcccaccaccaccacctcgcgccgcgcacgcccgaggtgcgggccgcggccgccggcgcgctcgggaaccatgttgtgagcggcagcccggccggagccgcgggcgcagggccggccgcccccgccgagggcgcgcccggagcggcgccggagccgccccccgcaggtagagcgcggccggggggcggggggccgcagcgcccgggccccccctcaccgcgccgcccccttgtccccgcagggcccgcgccgcccgccgcgaagctgaggccgccgcccgagggcagcgcgggggcctgcgccccggtgcccgccgccgccgccgtcgccgcggggcccgagcccgcccccgccggccccgccaagcccgccggccccgccgcgctggccgcccgcgccggccccggccccgggcccggccccggccccggccccggccctggcaagcccgccggccccggcgccgcgcaaactttgaatgggagcgccgcgctgctgaactcgcaccacgccgccgcacctgctgtcagcctggtcaacaacgggcccgccgcgctgctgccgctgcccaagcccgccgcccccggcactgtcatccagacgccccccttcgtgggcgccgccgcgccccccgcgcccgccgcgccctcgccccccgccgcccccgcgcccgccgcccccgccgccgccccgcccccgccaccccccgcgcccgccaccctggcccggccgcccggccaccccgccggacccccgaccgccgcgcccgccgtgccgccccccgccgccgcccagaacgggggcagcgccggggcagcccccgcccccgccccggccgccgggggccccgctggggtcagcggccagcccgggcccggcgcggcggctgcggcgccggcgccgggggtcaaggccgagtcgcccaagagggtggtgcaggcggcgcccccggcggcgcagaccctggcggccagcggcccggccagcacggcggccagcatggtcatcgggccaactatgcaaggggcgctgcccagcccggccgccgtcccgccgcccgcccccgggacccccaccgggctgcccaaaggcgcggccggcgcagtgacccagagcctgtcccggacgcccacggccaccaccagcgggattcgggccaccctgacgcccaccgtgctggccccccgcttgccgcagccgcctcagaacccgaccaacatccagaacttccagctgcccccaggaatggtcctcgtccgaagtgagaatgggcagttgttaatgattcctcagcaggccttggcccagatgcaggcgcaggcccatgcccagcctcagaccaccatggcgcctcgccctgccacccccacaagtgcccctcccgtccagatctccaccgtacaggcacctggaacacctatcattgcacggcaggtgaccccaactaccataattaagcaagtgtctcaggcccagacaacggtgcagcccagtgcaaccctgcagcgctcgcccggcgtccagcctcagctcgttctgggtggcgctgcccagacggcttcacttgggacggcgacggctgttcagacggggactcctcagcgcacggtaccaggggcgaccaccacttcctcagctgccacggaaactatggaaaacgtgaagaaatgtaaaaatttcctatctacgttaataaaactggcttcatctggcaagcagtctacagagacagcagctaatgtgaaagagctcgtgcagaatttactggtcatccagcagcctccgaagccaggagccctgatccggcccccgcaggtgacgttgacgcagacacccatggtcgccctgcggcagcctcacaaccggatcatgctcaccacgcctcagcagatccagctgaacccactgcagccagtccctgtggtgaaacccgccgtgttacctggaaccaaagccctttctgctgtctcggcacaagcagctgctgcacagaaaaataaactcaaggagcctgggggaggttcgtttcgggacgatgatgacattaatgatgttgcatcgatggctggagtaaacttgtcagaagaaagtgcaagaatattagccacgaactctgaattggtgggcacgctaacgcggtcctgtaaagatgaaaccttcctcctccaagcgcctttgcagagaagaatattagaaataggtaaaaaacatggtataacggaattacatccagatgtagtaagttatgtatcacatgccacgcaacaaaggctacagaatcttgtagagaaaatatcagaaacagctcagcagaagaacttttcttacaaggatgacgacagatatgagcaggcgagtgacgtccgggcacagctcaagttttttgaacagcttgatcaaatcgaaaagcagaggaaggatgagcaggagcgggagatcctgatgagggcagcaaagtctcggtcaagacaagaagatccagaacagttaaggctgaaacagaaggcaaaggagatgcagcaacaggaactggcacaaatgagacagcgggacgccaacctcacagcactagcagcgatcgggcccaggaaaaagaggaaagtggactgtccggggccgggctcaggagcagaggggtcgggccccggctcagtggtcccaggcagctcgggtgtcggaacccccagacagttcacgcgacaaagaatcacgcgggtcaacctcagggacctcatattttgtttagaaaatgaacgtgagacaagccattcactgctgctctacaaagcattccttaagtgacacaggaggacgcctggggactttttatatatttgcagattacgcctttttgtaacgagcaaatgggatattgtttaaaaaacagccacctctttacaatggaacagttttatattcctgtttctaaatcagctcttcagtgtgaaagaaaacacgtttctgtaacagagagaacacaaaggcctgtggatactcttaaaggacaattaaatcttaactcatcttgattgagtggccttcctgccaaacaagccatatataaagactgatggaatcgttagcaaataattagctgccctctgtcaactcatagcagtttctgcattatttgtgcattttggtttagttctacctaacttactatgtaggtgtatgtctacagccgatgacctcatttcgtttattttatttttgtaatagtcagttggcaaagcaaactgattttttagactatttatcttccttcccttcccctcccaccccgctctcctctctgccccctgccctcccctcccctcccttcccctccactccgctgagaatcctggaggaatacacaattcatcgttgcacccccacctcagagtgtaatcgcatttctgcttggtagaggccgagcccagcaaaggtggctccttctgaatgtgtggtcagcatctgtacaaatgcattttatttgctatagtttgtaaagctgtaaagttaaaagagatgaaaaccttttcagcataaatatattttacttgcactgtgttttttagctaaaagtgaaaacctagattaaataaaatcaaagttgagaagaatcatcaaaagactgtttctcggtgtgaatcaagtgttgaaaaatggttggtgtattttgtcagtaattgtacataacttttggcacatgacatagaaatggctatgtaaactataattattttgctaagagactgtatgcaagccttgggccgactttacagacgtccagagcaaagccccttctttgtacctatttttttattacaaatatactaattggttctttctattttcagaggttattgtatgaaattgtctattgatagtacttttatgactgtaaatactctggctttctccgtgtgaattctcacattagactttaattcgagcgcgtgtgaactgaacgctgatcagtattttttatcaacacctgagaactgttacaccttttattttgtcttttaggaaatccctgtctttccattttttcatgtaaattttgcacagttacttgttcatatgtaaatattttactttcagaaatgaagtttttaattgctattgttttatataggattgaaagaaaattaactcctttattaaaaacaaatttatctgtaaaaaaaaaaaaaaaaaaaa

SEQ ID NO: 2 - DNA, 4437 bp, Artificial Sequence
  Synthetic
atggcggcgggctcggatctgctggacgaggtcttcttcaacagcgaggtggacgagaaagtggtgagcgacctggtgggctcgctggagtcgcagctggcggccagcgcggcccaccaccaccacctcgcgccgcgcacgcccgaggtgcgggccgcggccgccggcgcgctcgggaaccatgttgtgagcggcagcccggccggagccgcgggcgcagggccggccgcccccgccgagggcgcgcccggagcggcgccggagccgccccccgcaggtagagcgcggccggggggcggggggccgcagcgcccgggccccccctcaccgcgccgcccccttgtccccgcagggcccgcgccgcccgccgcgaagctgaggccgccgcccgagggcagcgcgggggcctgcgccccggtgcccgccgccgccgccgtcgccgcggggcccgagcccgcccccgccggccccgccaagcccgccggccccgccgcgctggccgcccgcgccggccccggccccgggcccggccccggccccggccccggccctggcaagcccgccggccccggcgccgcgcaaactttgaatgggagcgccgcgctgctgaactcgcaccacgccgccgcacctgctgtcagcctggtcaacaacgggcccgccgcgctgctgccgctgcccaagcccgccgcccccggcactgtcatccagacgccccccttcgtgggcgccgccgcgccccccgcgcccgccgcgccctcgccccccgccgcccccgcgcccgccgcccccgccgccgccccgcccccgccaccccccgcgcccgccaccctggcccggccgcccggccaccccgccggacccccgaccgccgcgcccgccgtgccgccccccgccgccgcccagaacgggggcagcgccggggcagcccccgcccccgccccggccgccgggggccccgctggggtcagcggccagcccgggcccggcgcggcggctgcggcgccggcgccgggggtcaaggccgagtcgcccaagagggtggtgcaggcggcgcccccggcggcgcagaccctggcggccagcggcccggccagcacggcggccagcatggtcatcgggccaactatgcaaggggcgctgcccagcccggccgccgtcccgccgcccgcccccgggacccccaccgggctgcccaaaggcgcggccggcgcagtgacccagagcctgtcccggacgcccacggccaccaccagcgggattcgggccaccctgacgcccaccgtgctggccccccgcttgccgcagccgcctcagaacccgaccaacatccagaacttccagctgcccccaggaatggtcctcgtccgaagtgagaatgggcagttgttaatgattcctcagcaggccttggcccagatgcaggcgcaggcccatgcccagcctcagaccaccatggcgcctcgccctgccacccccacaagtgcccctcccgtccagatctccaccgtacaggcacctggaacacctatcattgcacggcaggtgaccccaactaccataattaagcaagtgtctcaggcccagacaacggtgcagcccagtgcaaccctgcagcgctcgcccggcgtccagcctcagctcgttctgggtggcgctgcccagacggcttcacttgggacggcgacggctgttcagacggggactcctcagcgcacggtaccaggggcgaccaccacttcctcagctgccacggaaactatggaaaacgtgaagaaatgtaaaaatttcctatctacgttaataaaactggcttcatctggcaagcagtctacagagacagcagctaatgtgaaagagctcgtgcagaatttactggatggaaaaatagaagcagaagatttcacaagcaggttataccgagaacttaattcttcacctcaaccttaccttgtgcctttcctgaagaggagcttacccgccttgagacagctgacccccgactccgcggccttcatccagcagcctccgaagccaggagccctgatccggcccccgcaggtgacgttgacgcagacacccatggtcgccctgcggcagcctcacaaccggatcatgctcaccacgcctcagcagatccagctgaacccactgcagccagtccctgtggtgaaacccgccgtgttacctggaaccaaagccctttctgctgtctcggcacaagcagctgctgcacagaaaaataaactcaaggagcctgggggaggttcgtttcgggacgatgatgacattaatgatgttgcatcgatggctggagtaaacttgtcagaagaaagtgcaagaatattagccacgaactctgaattggtgggcacgctaacgcggtcctgtaaagatgaaaccttcctcctccaagcgcctttgcagagaagaatattagaaataggtaaaaaacatggtataacggaattacatccagatgtagtaagttatgtatcacatgccacgcaacaaaggctacagaatcttgtagagaaaatatcagaaacagctcagcagaagaacttttcttacaaggatgacgacagatatgagcaggcgagtgacgtccgggcacagctcaagttttttgaacagcttgatcaaatcgaaaagcagaggaaggatgagcaggagcgggagatcctgatgagggcagcaaagtctcggtcaagacaagaagatccagaacagttaaggctgaaacagaaggcaaaggagatgcagcaacaggaactggcacaaatgagacagcgggacgccaacctcacagcactagcagcgatcgggcccaggaaaaagaggaaagtggactgtccggggccgggctcaggagcagaggggtcgggccccggctcagtggtcccaggcagctcgggtgtcggaacccccagacagttcacgcgacaaagaatcacgcgggtcaacctcagggacctcatattttgtttagaaaatgaacgtgagacaagccattcactgctgctctacaaagcattccttaagtgacacaggaggacgcctggggactttttatatatttgcagattacgcctttttgtaacgagcaaatgggatattgtttaaaaaacagccacctctttacaatggaacagttttatattcctgtttctaaatcagctcttcagtgtgaaagaaaacacgtttctgtaacagagagaacacaaaggcctgtggatactcttaaaggacaattaaatcttaactcatcttgattgagtggccttcctgccaaacaagccatatataaagactgatggaatcgttagcaaataattagctgccctctgtcaactcatagcagtttctgcattatttgtgcattttggtttagttctacctaacttactatgtaggtgtatgtctacagccgatgacctcatttcgtttattttatttttgtaatagtcagttggcaaagcaaactgattttttagactatttatcttccttcccttcccctcccaccccgctctcctctctgccccctgccctcccctcccctcccttcccctccactccgctgagaatcctggaggaatacacaattcatcgttgcacccccacctcagagtgtaatcgcatttctgcttggtagaggccgagcccagcaaaggtggctccttctgaatgtgtggtcagcatctgtacaaatgcattttatttgctatagtttgtaaagctgtaaagttaaaagagatgaaaaccttttcagcataaatatattttacttgcactgtgttttttagctaaaagtgaaaacctagattaaataaaatcaaagttgagaagaatcatcaaaagactgtttctcggtgtgaatcaagtgttgaaaaatggttggtgtattttgtcagtaattgtacataacttttggcacatgacatagaaatggctatgtaaactataattattttgctaagagactgtatgcaagccttgggccgactttacagacgtccagagcaaagccccttctttgtacctatttttttattacaaatatactaattggttctttctattttcagaggttattgtatgaaattgtctattgatagtacttttatgactgtaaatactctggctttctccgtgtgaattctcacattagactttaattcgagcgcgtgtgaactgaacgctgatcagtattttttatcaacacctgagaactgttacaccttttattttgtcttttaggaaatccctgtctttccattttttcatgtaaattttgcacagttacttgttcatatgtaaatattttactttcagaaatgaagtttttaattgctattgttttatataggattgaaagaaaattaactcctttattaaaaacaaatttatctgtaaaaaaaaaaaaaaaaaaaa

SEQ ID NO: 3 - DNA, 3350 bp, Artificial Sequence
  Synthetic
atggcggcgggctcggatctgctggacgaggtcttcttcaacagcgaggtggacgagaaagtggaatggtcctcgtccgaagtgagaatgggcagttgttaatgattcctcagcaggccttggcccagatgcaggcgcaggcccatgcccagcctcagaccaccatggcgcctcgccctgccacccccacaagtgcccctcccgtccagatctccaccgtacaggcacctggaacacctatcattgcacggcaggtgaccccaactaccataattaagcaagtgtctcaggcccagacaacggtgcagcccagtgcaaccctgcagcgctcgcccggcgtccagcctcagctcgttctgggtggcgctgcccagacggcttcacttgggacggcgacggctgttcagacggggactcctcagcgcacggtaccaggggcgaccaccacttcctcagctgccacggaaactatggaaaacgtgaagaaatgtaaaaatttcctatctacgttaataaaactggcttcatctggcaagcagtctacagagacagcagctaatgtgaaagagctcgtgcagaatttactggatggaaaaatagaagcagaagatttcacaagcaggttataccgagaacttaattcttcacctcaaccttaccttgtgcctttcctgaagaggagcttacccgccttgagacagctgacccccgactccgcggccttcatccagcagagccagcagcagccgccaccgcccacctcgcaggccaccactgcgctcacggccgtggtgctgagtagctcggtccagcgcacggccgggaagacggcggccaccgtgaccagtgccctccagccccctgtgctcagcctcacgcagcccacgcaggtcggcgtcggcaagcaggggcaacccacaccgctggtcatccagcagcctccgaagccaggagccctgatccggcccccgcaggtgacgttgacgcagacacccatggtcgccctgcggcagcctcacaaccggatcatgctcaccacgcctcagcagatccagctgaacccactgcagccagtccctgtggtgaaacccgccgtgttacctggaaccaaagccctttctgctgtctcggcacaagcagctgctgcacagaaaaataaactcaaggagcctgggggaggttcgtttcgggacgatgatgacattaatgatgttgcatcgatggctggagtaaacttgtcagaagaaagtgcaagaatattagccacgaactctgaattggtgggcacgctaacgcggtcctgtaaagatgaaaccttcctcctccaagcgcctttgcagagaagaatattagaaataggtaaaaaacatggtataacggaattacatccagatgtagtaagttatgtatcacatgccacgcaacaaaggctacagaatcttgtagagaaaatatcagaaacagctcagcagaagaacttttcttacaaggatgacgacagatatgagcaggcgagtgacgtccgggcacagctcaagttttttgaacagcttgatcaaatcgaaaagcagaggaaggatgagcaggagcgggagatcctgatgagggcagcaaagtctcggtcaagacaagaagatccagaacagttaaggctgaaacagaaggcaaaggagatgcagcaacaggaactggcacaaatgagacagcgggacgccaacctcacagcactagcagcgatcgggcccaggaaaaagaggaaagtggactgtccggggccgggctcaggagcagaggggtcgggccccggctcagtggtcccaggcagctcgggtgtcggaacccccagacagttcacgcgacaaagaatcacgcgggtcaacctcagggacctcatattttgtttagaaaatgaacgtgagacaagccattcactgctgctctacaaagcattccttaagtgacacaggaggacgcctggggactttttatatatttgcagattacgcctttttgtaacgagcaaatgggatattgtttaaaaaacagccacctctttacaatggaacagttttatattcctgtttctaaatcagctcttcagtgtgaaagaaaacacgtttctgtaacagagagaacacaaaggcctgtggatactcttaaaggacaattaaatcttaactcatcttgattgagtggccttcctgccaaacaagccatatataaagactgatggaatcgttagcaaataattagctgccctctgtcaactcatagcagtttctgcattatttgtgcattttggtttagttctacctaacttactatgtaggtgtatgtctacagccgatgacctcatttcgtttattttatttttgtaatagtcagttggcaaagcaaactgattttttagactatttatcttccttcccttcccctcccaccccgctctcctctctgccccctgccctcccctcccctcccttcccctccactccgctgagaatcctggaggaatacacaattcatcgttgcacccccacctcagagtgtaatcgcatttctgcttggtagaggccgagcccagcaaaggtggctccttctgaatgtgtggtcagcatctgtacaaatgcattttatttgctatagtttgtaaagctgtaaagttaaaagagatgaaaaccttttcagcataaatatattttacttgcactgtgttttttagctaaaagtgaaaacctagattaaataaaatcaaagttgagaagaatcatcaaaagactgtttctcggtgtgaatcaagtgttgaaaaatggttggtgtattttgtcagtaattgtacataacttttggcacatgacatagaaatggctatgtaaactataattattttgctaagagactgtatgcaagccttgggccgactttacagacgtccagagcaaagccccttctttgtacctatttttttattacaaatatactaattggttctttctattttcagaggttattgtatgaaattgtctattgatagtacttttatgactgtaaatactctggctttctccgtgtgaattctcacattagactttaattcgagcgcgtgtgaactgaacgctgatcagtattttttatcaacacctgagaactgttacaccttttattttgtcttttaggaaatccctgtctttccattttttcatgtaaattttgcacagttacttgttcatatgtaaatattttactttcagaaatgaagtttttaattgctattgttttatataggattgaaagaaaattaactcctttattaaaaacaaatttatctgtaaaaaaaaaaaaaaaaaaaa

SEQ ID NO: 4 - DNA, 2767 bp, Artificial Sequence
  Synthetic
gcgcgaggtggctcagccgcaagatggcggcgctggcggaggagcagacggaggtggcggtcaagctagagcctgagggaccgccaacgctgctacctccgcaggcgggggacggcgcaggcgagggtagcggcggcactaccaacaacggccccaacggcggcggcgggaacgttgcggcgtcgtcgtccactggcggggatggcgggacccccaagcccacggtggctgtctccgccgctgccccggcgggggcggccccggtgcccgccgctgctccggacgccggcgctccgcatgaccgacagactctactggccgtgctgcagttcctacggcagagcaaactccgcgaggccgaagaggcgctgcgccgtgaggccgggctgctggaggaggcagtggcgggctccggagccccgggagaggtggacagcgccggcgctgaggtgaccagcgcgcttctcagccgggtgaccgcctcggcccctggccctgcggcccccgaccctccgggcactggcgcttcgggggccacggtcgtctcaggttcagcctcaggtcctgcggctccgggtaaagttggaagtgttgctgtggaagaccagccagatgtcagtgccgtgttgtcagcctacaaccaacaaggagatcccacaatgtatgaagaatactatagtggactgaaacacttcattgaatgttccctggactgccatcgggcagagttgtcccaacttttttatcctctgtttgtgcacatgtacttggagctagtctacaatcaacatgagaatgaagcaaagtcattctttgagaagtattttttggtttattaaaagaaccagaaattgaggtacctttggatgacgaggatgaagagggagaaaatgaagaaggaaaacctaaaaagaagaagcctaaaaaagatagtattggatccaaaagcaaaaaacaagatcccaatgctccacctcagaacagaatccctcttcctgagttgaaagattcagataagttggataagataatgaatatgaaagaaaccaccaaacgagtgcgccttgggccggactgcttaccctccatttgtttctatacatttctcaatgcttaccagggtctcactgcagtggatgtcactgatgattctagtctgattgctggaggttttgcagattcaactgtcagagtgtggtcggtaacacccaaaaagcttcgtagtgtcaaacaagcatcagatcttagtcttatagacaaagaatcagatgatgtcttagaaagaatcatggatgagaaaacagcaagtgagttgaagattttgtatggtcacagtgggcctgtctacggagccagcttcagtccggataggaactatctgctttcctcttcagaggacggaactgttagattgtggagccttcaaacatttacttgtttggtgggatataaaggacacaactatccagtatgggacacacaattttctccatatggatattattttgtgtcagggggccatgaccgagtagctcggctctgggctacagaccactatcagcctttaagaatatttgccggccatcttgctgatgtgaattgtaccagattccatccaaattctaattatgttgctacgggctctgcagacagaactgtgcggctctgggacgtcctgaatggtaactgtgtaaggatcttcactggacacaagggaccaattcattccttgacattttctcccaatgggagattcctggctacaggagcaacagatggcagagtgcttctttgggatattggacatggtttgatggttggagaattaaaaggccacactgatacagtctgttcacttaggtttagtagagatggtgaaattttggcatcaggttcaatggataatacagttcgattatgggatgctatcaaagcctttgaagatttagagaccgatgactttactacagccactgggcatataaatttacctgagaattcacaggagttattgttgggaacatatatgaccaaatcaacaccagttgtacaccttcattttactcgaagaaacctggttctagctgcaggagcttatagtccacaataaaccatcggtattaaagaccttttggaagctactgtttttaaaaagggagactaaaagcaaatacctcagtgattaatatttaagctacagagaatgtttttgtctatatggatctggaagtatgctgcttggaaaaatctgaacaggacagttccacgtttctatagcaaccacatttgactaatttccgttagttgaataagaggtattatgatcatggaggggacatttatggtgctttggattgtgtggaaactatgcattttctgttcaaatgctattttaatttattacatttagaaaaaaagttgatttcaataattcatcctgcttcaagattcaaattcagaaatatactatcatcttgaattttagctgaagaatcctatgagcatgtatgtttctgctgtaaaaacgtagttactgtatggcactcaaaaactatgttaaatgatccactaactttttttttcttggcccatgattaatggaatgtatgtaactaggtagggttcctttcttagatctagaggaagtacagccacccactgacatctgaatttatatacctgttgagttttgagtgcacccaaacactcgataaaccaggtgaagaaatttagcttccatgttctacttcagctaaaacagctacat

SEQ ID NO: 5 - DNA, 2549 bp, Artificial Sequence
  Synthetic
atgagtgtgagctcgtgagtgggcgccgccgccaccgcccccgccgccgtcgtctcggtagcagccttcgccacgccggggtcttcagctccactggggccatgtcagagcgagaagagcggcggtttgtggagatccctcgggagtctgtccggctcatggcggagagcacgggcctggagctgagcgatgaggtggcggcgctgctcgcagaggacgtgtgctatcgtctgagagaggccacgcagaatagctctcagttcatgaagcacaccaaacgccggaagctgacggttgaggacttcaacagggccctcagatggagcagcgtggaggctgtgtgtggttacggatcacaggaggcactgcccatgcgccccgccagggagggtgaactctactttcctgaggatcgagaggtgaacctggtggagctggccctggctaccaacatccccaaaggctgtgctgagacagctgtcagagttcatgtctcctacctggatggcaaagggaacctggcacctcaaggatcgggtaaggggtgatgtaggaaacaggctctttggatgaattttctcccttaggttctgagggtggtgcctatgtgcccccgagtctgcgtctaacatgtgtttacccatgcctgccttgtgccatggtctgagtgggcgctgggctctgcatggagggctcagagttggagatgggggcccagacctgtaactagtcataatgcagcatgttggatgctaagacagaagtctgggcagcatgctggggcggtgtttcacccccagggtatgctgagcagagcttcacagagcctgaagctctcaggagtccgtctggcagagggtgggtggaagacaggacagagcacagaggtgtgcagagcctagatggtcagggctgagcaggctctaagagcagtctcttgccctggttgtcctgtcagaaaggcttcttgtggatgtgtgtggggatggtggttgagggggaggaggctggagaggccaggagagggccagctctccacctgtccctgcttcctgcctgtcctctggcagtgcccagtgctgtgtcttcactgacagatgaccttctcaagtactatcaccaggtgactcgtgctgtgctaggggatgatccgcaactgatgaaggttgcactccaggacttgcagacgaactccaagattggggcactcctgccttactttgtttatgtggtcagtggggtgaaatctgtaagccatgacctggagcaactgcaccggctgctgcaggtggcacggagcctatttcgtaatccgcacctgtgcttggggccctatgtccgctgtctggtgggcagtgtcctctactgtgtcctggagccactggctgcctccatcaaccccctgaatgaccactggactctgcgggatggggctgccctcctgctcagccacatcttctggactcatggggaccttgtaagtggcctctatcagcatatcctgctatccctgcagaagatcctggcagatcctgtgcggccgctctgctgccactatggagccgtggtggggctgcatgctcttggctggaaggcagtagaacgagtcctgtacccacacctgtccacctactggacaaacttgcaggctgtgctggatgattattcagtatctaatgcccaggtcaaagcagatggacacaaagtctatggagccattctggtggcggtagagcgactgctgaagatgaaggcccaggcagcagagcccaacaggggtggcccaggtggcagggggtgccggcgcctggacgacctgccatgggacagccttctctttcaagagtcgtcctccgggggcggtgcagaacccagctttgggtccggcctcccgctgccgccagggggcgcggggccggaggacccttctctttcggtgaccctggccgacatctaccgggagctctacgccttcttcggtgacagcttggccacacgctttggcaccggccagcctgcacccacggctccgcggccgcccggggacaagaaggagccggcggcagccccggactcggtgcggaagatgccgcagctgacggcaagcgccatagtcagcccgcacggcgacgagagcccccggggcagcggcggaggcggccccgcgtcggcctctgggcccgccgcctctgagagcaggcccttgccgcgcgtgcatcgggcgcgcggggcaccccggcagcagggccccgggaccggcacccgcgacgttttccagaagagccgtttcgccccgcgcggcgccccgcactttcgtttcatcatagccgggcggcaggctgggaggcgctgccgcgggcgccttttccagactgccttccccgcgccgtacgggcctagcccggcctcgcgctacgtgcagaaactgcccatgatcggccgtaccagccgccccgcccgccggtgggcgctctcggactactcgctgtacttgccgctctgagtcagtggccccttcgttccttgtaaataaatcccgcccccggaaa

SEQ ID NO: 6 - DNA, 2128 bp, Artificial Sequence
  Synthetic
atgagtgtgagctcgtgagtgggcgccgccgccaccgcccccgccgccgtcgtctcggtagcagccttcgccacgccggggtcttcagctccactggggccatgtcagagcgagaagagcggcggtttgtggagatccctcgggagtctgtccggctcatggcggagagcacgggcctggagctgagcgatgaggtggcggcgctgctcgcagaggacgtgtgctatcgtctgagagaggccacgcagaatagctctcagttcatgaagcacaccaaacgccggaagctgacggttgaggacttcaacagggccctcagatggagcagcgtggaggctgtgtgtggttacggatcacaggaggcactgcccatgcgccccgccagggagggtgaactctactttcctgaggatcgagaggtgaacctggtggagctggccctggctaccaacatccccaaaggctgtgctgagacagctgtcagagttcatgtctcctacctggatggcaaagggaacctggcacctcaaggatcggaaaggcttcttgtggatgtgtgtggggatggtggttgagggggaggaggctggagaggccaggagagggccagctctccacctgtccctgcttcctgcctgtcctctggcagtgcccagtgctgtgtcttcactgacagatgaccttctcaagtactatcaccaggtgactcgtgctgtgctaggggatgatccgcaactgatgaaggttgcactccaggacttgcagacgaactccaagattggggcactcctgccttactttgtttatgtggtcagtggggtgaaatctgtaagccatgacctggagcaactgcaccggctgctgcaggtggcacggagcctatttcgtaatccgcacctgtgcttggggccctatgtccgctgtctggtgggcagtgtcctctactgtgtcctggagccactggctgcctccatcaaccccctgaatgaccactggactctgcgggatggggctgccctcctgctcagccacatcttctggactcatggggaccttgtaagtggcctctatcagcatatcctgctatccctgcagaagatcctggcagatcctgtgcggccgctctgctgccactatggagccgtggtggggctgcatgctcttggctggaaggcagtagaacgagtcctgtacccacacctgtccacctactggacaaacttgcaggctgtgctggatgattattcagtatctaatgcccaggtcaaagcagatggacacaaagtctatggagccattctggtggcggtagagcgactgctgaagatgaaggcccaggcagcagagcccaacaggggtggcccaggtggcagggggtgccggcgcctggacgacctgccatgggacagccttctctttcaagagtcgtcctccgggggcggtgcagaacccagctttgggtccggcctcccgctgccgccagggggcgcggggccggaggacccttctctttcggtgaccctggccgacatctaccgggagctctacgccttcttcggtgacagcttggccacacgctttggcaccggccagcctgcacccacggctccgcggccgcccggggacaagaaggagccggcggcagccccggactcggtgcggaagatgccgcagctgacggcaagcgccatagtcagcccgcacggcgacgagagcccccggggcagcggcggaggcggccccgcgtcggcctctgggcccgccgcctctgagagcaggcccttgccgcgcgtgcatcgggcgcgcggggcaccccggcagcagggccccgggaccggcacccgcgacgttttccagaagagccgtttcgccccgcgcggcgccccgcactttcgtttcatcatagccgggcggcaggctgggaggcgctgccgcgggcgccttttccagactgccttccccgcgccgtacgggcctagcccggcctcgcgctacgtgcagaaactgcccatgatcggccgtaccagccgccccgcccgccggtgggcgctctcggactactcgctgtacttgccgctctgagtcagtggccccttcgttccttgtaaataaatcccgcccccggaaa

SEQ ID NO: 7 - DNA, 1393 bp, Artificial Sequence
  Synthetic
gcacactacgccagaacaagatggccgacgcggcggccacagctggggccggtggctccggaacgagatcgggaagtaaacagtccactaaccctgccgataactatcatctggcccggaggagaaccctgcaggtggttgtgagctccttgctgacagaggcagggtttgagagtgccgagaaagcatccgtggaaacgctgacagagatgctgcagagctacatttcagaaattgggagaagtgccaagtcttactgtgagcacacagccaggacccagcccacactgtccgatatcgtggtcacacttgttgagatgggtttcaatgtggacactctccctgcttatgcaaaacggtctcagaggatggtcatcactgctcctccggtgaccaatcagccagtgacccccaaggccctcactgcagggcaggaccgaccccacccgccgcacatccccagccattttcctgagttccctgatccccacacctacatcaaaactccggaggattctggagccgagaaggagaacacctctgtcctgcagcagaacccctccttgtcgggtagccggaatggggaggagaacatcatcgataacccttatctgcggccggtgaagaagcccaagatccgcaggaagaagccagatacattctagagaattgtgagactttgccctaagctgccaagtgctccccaaggagatcggtcaccaggagagcagccacaaaggtcaagaaagacacacacggaagcaaacccaggctctgtgctccttccagcccttgctggtgcagatgctaccccacaagcttgtcagatcgcccaggtgacgggccaggcgcagccatcgggaaggtcattcagccaacccagaggatgtagaccctgtcctcaagaagtgcagggcgagttctgccgtgccctctgaaatactctccctcctctcagagccctccctcgagagcctgagtgcaccatgtcttgtggcctcgcctgaagccttcctctggcttcacagaacgctctggaactctggggtctggtcagggagtgtgtcctcagcttgtctggaggaggcctgcatccctcctgagctcttggaggtgcccaggaaccctgcttctcctcacagggcctggccatatagccagctccaacccaaagccctgcacagtgcctcacccactcccttctcctggtctctcctcaaaagaattttacatgattttaaaataataatagctttcatttacatagtgcttacatttatatagcacttaactatgtgcgatgtactaatttaagtactttatattaactcatataataaatggacacaacacaaaactcaaaaggtacaaaagaataaaacagttaaaaact

SEQ ID NO: 8 - DNA, 1532 bp, Artificial Sequence
  Synthetic
gcacactacgccagaacaagatggccgacgcggcggccacagctggggccggtggctccggaacgagatcgggaagtaaacagtccactaaccctgccgataactatcatctggcccggaggagaaccctgcaggtggttgtgagctccttgctgacagaggcagggtttgagagtgccgagaaagcatccgtggaaacgctgacagagatgctgcagagctacatttcagaaattgggagaagtgccaagtcttactgtgagcacacagccaggacccagcccacactgtccgatatcgtggtcacacttgttgagatgggtttcaatgtggacactctccctgcttatgcaaaacggtctcagaggatggtcatcactgctctgattgctgccagacctttcaccatcccctacctgacagctcttcttccgtctgaactggagatgcaacaaatggaagagcagattcctcggagcaggatgaacagacagacacagagaaccttgctcttcatatcagcatgatagagtctcgctccgtcacccaggctggagtgcagtggcaagatcttggctcactgcaacctccgcctcctgggttcaagcgattctccagcctcagcctcctgagtagctggaattacaggaggattctggagccgagaaggagaacacctctgtcctgcagcagaacccctccttgtcgggtagccggaatggggaggagaacatcatcgataacccttatctgcggccggtgaagaagcccaagatccgcaggaagaagccagatacattctagagaattgtgagactttgccctaagctgccaagtgctccccaaggagatcggtcaccaggagagcagccacaaaggtcaagaaagacacacacggaagcaaacccaggctctgtgctccttccagcccttgctggtgcagatgctaccccacaagcttgtcagatcgcccaggtgacgggccaggcgcagccatcgggaaggtcattcagccaacccagaggatgtagaccctgtcctcaagaagtgcagggcgagttctgccgtgccctctgaaatactctccctcctctcagagccctccctcgagagcctgagtgcaccatgtcttgtggcctcgcctgaagccttcctctggcttcacagaacgctctggaactctggggtctggtcagggagtgtgtcctcagcttgtctggaggaggcctgcatccctcctgagctcttggaggtgcccaggaaccctgcttctcctcacagggcctggccatatagccagctccaacccaaagccctgcacagtgcctcacccactcccttctcctggtctctcctcaaaagaattttacatgattttaaaataataatagctttcatttacatagtgcttacatttatatagcacttaactatgtgcgatgtactaatttaagtactttatattaactcatataataaatggacacaacacaaaactcaaaaggtacaaaagaataaaacagttaaaaact

SEQ ID NO: 9 - DNA, 891 bp, Artificial Sequence
  Synthetic
gttcgccgcctctcccaccggcccgatgagctgcagcggctccggcgcggaccccgaggcggcgccggcctccgccgcctcggccccgggccccgcgcccccggtctcggctcccgccgcgctgccctccagcaccgccgcggagaacaaggccagccccgcggggacagcggggggacctggggctggagcagctgctgggggcacgggacccttggcggcgcgggccggggagccagctgagcggcgtggggcggctccggtgtcggcgggtggcgcggcgcccccggagggggccatatctaacggggtttacgtactgccgagcgcggccaacggagacgtgaagcccgtggtgtccagcacgcctttggtggacttcttgatgcagctggaagattacacgcctacggtgggcttccgcccgaacaaggccacctagcctgctgacaaaactttcagccacatcgtgcttttcagcgttctcttccatttgctcccctagtcgctcttctgtgtttgccctctgctcacccaaactatcccagatgcagtgactggttactacctgaaccgtgctggctttgaggcctcagacccacgcataattcggctcatctccttagctgcccagaaattcatctcagatattgccaatgatgccctacagcactgcaaaatgaagggcacggcctccggcagctcccggagcaagagcaaggaccgcaagtacactctaaccatggaggacttgacccctgccctcagcgagtatggcatcaatgtgaagaagccgcactacttcacctgagccacccaacctaaatgtacttatctgtccccatgtccccacaccagcctgttttcataataaactttattgtgacaggc

SEQ ID NO: 10 - DNA, 3304 bp, Artificial Sequence
  Synthetic
gcgagtgttgaaagtcggtggcgtaggtcgtcgtcctggatgctggcgagatagatgttatcttccagaggaagaggaggaggcggcgaagcgttttcccagcctcagtctctctttcgttttccttttcccttcccccaaccctccgcccttctctaaatcagccggccttccttgacctcagtgacccgtctggccccgcccaccctcgtcgacgtgattcccgccgtgagaagactccaacttcacctttgaagatgaaaccagggcgcccacgaataaaaaaagatgagaagcagaacttactatccgttggcgattaccgacaccgtagaacagagcaagaggaggatgaagagctattaacagaaagctccaaagcaaccaatgtttgcactcgatttgaagactctccatcgtatgtaaaatggggtaaactgagagattatcaggtccgaggattaaactggctcatttctttgtatgagaatggcatcaatggtatccttgcagatgaaatgggcctaggaaagactcttcaaacaatttctcttcttgggtacatgaaacattatagaaacattcctgggcctcatatggttttggttcctaagtctacattacacaactggatgagtgaattcaagagatgggtaccaacacttagatctgtttgtttgataggagataaagaacaaagagctgcttttgtcagagacgttttattaccgggagaatgggatgtatgtgtaacatcttatgaaatgcttattaaagagaagtctgtgttcaaaaaatttaattggagatacttagtaatagatgaagctcacaggatcaaaaatgaaaaatctaagttgtcagaaatagtgagggaattcaagactacaaatagactattattaactggaacacctcttcagaacaacttgcatgagctgtggtcacttcttaactttctgttgccagatgtgtttaattcagcagatgactttgattcctggtttgatacaaacaactgccttggggatcaaaaactagttgagaggcttcatatggttttgcgtccattcctccttcgtcgaattaaggctgatgttgaaaagagtttgcctccaaagaaggaagtaaaaatctatgtgggcctcagcaaaatgcaaagggaatggtatactcggatattaatgaaggatatagatatactcaactcagcaggcaagatggacaaaatgaggttattgaacatcctaatgcagttgagaaaatgttgtaatcatccatatctctttgatggagcagaacctggtccaccttatacaacagatatgcatctagtaaccaacagtggcaaaatggtggttttagacaagctgctccctaagttaaaagaacaaggttcacgagtactaatcttcagtcaaatgacaagggtattggacattttggaagattattgcatgtggagaaattatgagtactgcaggttggatggtcagacaccccatgatgagagacaagactccatcaatgcatacaatgaaccaaacagcacaaagtttgttttcatgttaagcacgcgtgctggtggtcttggcatcaatcttgcgactgctgatgtagtaattttgtatgattctgattggaatccccaagtagatcttcaggctatggaccgagcacatagaattgggcagactaagacagtcagagtgttccgctttataactgataacactgtagaagaaagaatagtagaacgtgctgagatgaaactcagactggattcaatagtcattcaacaagggaggcttgtggatcagaatctgaacaaaattgggaaagatgaaatgcttcaaatgattagacatggagcaacacatgtgtttgcttcaaaggaaagtgagatcactgatgaagatatcgatggtattttggaaagaggtgcaaagaagactgcagagatgaatgaaaagctctccaagatgggcgaaagttcacttagaaactttacaatggatacagagtcaagtgtttataacttcgaaggagaagactatagagaaaaacaaaagattgcattcacagagtggattgaaccacctaaacgagaaagaaaagccaactatgccgttgatgcatatttcagggaagctcttcgtgttagtgaacctaaagcacccaaggctcctcgacctccaaaacaacccaatgttcaggatttccagttctttcctccacgtttatttgaattactggaaaaagaaattctgttttacagaaaaactattgggtacaaggtacctcgaaatcctgagctgcctaacgcagcacaggcacaaaaagaagaacagcttaaaattgatgaagctgaatcccttaatgatgaagagttagaggaaaaagagaagcttctaacacagggatttaccaattggaataagagagattttaaccagtttatcaaagctaatgagaagtggggtcgtgatgatattgaaaatatagcaagagaagtagaaggcaaaactccagaagaagtcattgaatattcagctgtgttttgggaaaggtgcaacgagctccaggacatagagaagattatggctcagattgaaaggggagaggcgagaattcaaagaagaataagcatcaagaaagcacttgacacaaagattggacggtacaaagcaccttttcatcagctgagaatatcatatggtactaacaaaggaaaaaactatactgaagaagaagatcgttttctgatttgtatgcttcacaaacttggatttgacaaagaaaatgtttatgatgaattgcgacagtgtattcgcaactctcctcagttcagatttgactggtttcttaagtccagaactgcaatggagctccagaggagatgtaataccttaattactttgattgaaagagaaaacatggaactagaagaaaaggagaaggcagagaaaaagaaacgaggaccaaagccttcaacacagaaacgtaaaatggatggcgcacctgatggtcgaggaagaaaaaagaagctgaaactatgaatatgtttttgtttcataatcactaactttaaaccagtagttctttaatttacgggtcttcataagatgtactgtacaatgctcaattgttatgtcatttaaagacatcaggttcatctgtttactgagctagaaacatagtatgtagtttcacttttttaaatgcaacagctgtgctgaaatttttttatcattaacacttgaagtaataaaataggcttcatttattaaaaaaaaaaaaaaaa

SEQ ID NO: 11 - DNA, 3460 bp, Artificial Sequence
  Synthetic
gcgagtgttgaaagtcggtggcgtaggtcgtcgtcctggatgctggcgagatagatgttatcttccagaggaagaggaggaggcggcgaagcgttttcccagcctcagtctctctttcgttttccttttcccttcccccaaccctccgcccttctctaaatcagccggccttccttgacctcagtgacccgtctggccccgcccaccctcgtcgacgtgattcccgccgtgaggaaatatttgatgatgcgtcacctggaaagcaaaaggaaatccaagaaccagatcctacctatgaagaaaaaatgcaaactgaccgggcaaatagattcgagtatttattaaagcagacagaactttttgcacatttcattcaacctgctgctcagaagactccaacttcacctttgaagatgaaaccagggcgcccacgaataaaaaaagatgagaagcagaacttactatccgttggcgattaccgacaccgtagaacagagcaagaggaggatgaagagctattaacagaaagctccaaagcaaccaatgtttgcactcgatttgaagactctccatcgtatgtaaaatggggtaaactgagagattatcaggtccgaggattaaactggctcatttctttgtatgagaatggcatcaatggtatccttgcagatgaaatgggcctaggaaagactcttcaaacaatttctcttcttgggtacatgaaacattatagaaacattcctgggcctcatatggttttggttcctaagtctacattacacaactggatgagtgaattcaagagatgggtaccaacacttagatctgtttgtttgataggagataaagaacaaagagctgcttttgtcagagacgttttattaccgggagaatgggatgtatgtgtaacatcttatgaaatgcttattaaagagaagtctgtgttcaaaaaatttaattggagatacttagtaatagatgaagctcacaggatcaaaaatgaaaaatctaagttgtcagaaatagtgagggaattcaagactacaaatagactattattaactggaacacctcttcagaacaacttgcatgagctgtggtcacttcttaactttctgttgccagatgtgtttaattcagcagatgactttgattcctggtttgatacaaacaactgccttggggatcaaaaactagttgagaggcttcatatggttttgcgtccattcctccttcgtcgaattaaggctgatgttgaaaagagtttgcctccaaagaaggaagtaaaaatctatgtgggcctcagcaaaatgcaaagggaatggtatactcggatattaatgaaggatatagatatactcaactcagcaggcaagatggacaaaatgaggttattgaacatcctaatgcagttgagaaaatgttgtaatcatccatatctctttgatggagcagaacctggtccaccttatacaacagatatgcatctagtaaccaacagtggcaaaatggtggttttagacaagctgctccctaagttaaaagaacaaggttcacgagtactaatcttcagtcaaatgacaagggtattggacattttggaagattattgcatgtggagaaattatgagtactgcaggttggatggtcagacaccccatgatgagagacaagactccatcaatgcatacaatgaaccaaacagcacaaagtttgttttcatgttaagcacgcgtgctggtggtcttggcatcaatcttgcgactgctgatgtagtaattttgtatgattctgattggaatccccaagtagatcttcaggctatggaccgagcacatagaattgggcagactaagacagtcagagtgttccgctttataactgataacactgtagaagaaagaatagtagaacgtgctgagatgaaactcagactggattcaatagtcattcaacaagggaggcttgtggatcagaatctgaacaaaattgggaaagatgaaatgcttcaaatgattagacatggagcaacacatgtgtttgcttcaaaggaaagtgagatcactgatgaagatatcgatggtattttggaaagaggtgcaaagaagactgcagagatgaatgaaaagctctccaagatgggcgaaagttcacttagaaactttacaatggatacagagtcaagtgtttataacttcgaaggagaagactatagagaaaaacaaaagattgcattcacagagtggattgaaccacctaaacgagaaagaaaagccaactatgccgttgatgcatatttcagggaagctcttcgtgttagtgaacctaaagcacccaaggctcctcgacctccaaaacaacccaatgttcaggatttccagttctttcctccacgtttatttgaattactggaaaaagaaattctgttttacagaaaaactattgggtacaaggtacctcgaaatcctgagctgcctaacgcagcacaggcacaaaaagaagaacagcttaaaattgatgaagctgaatcccttaatgatgaagagttagaggaaaaagagaagcttctaacacagggatttaccaattggaataagagagattttaaccagtttatcaaagctaatgagaagtggggtcgtgatgatattgaaaatatagcaagagaagtagaaggcaaaactccagaagaagtcattgaatattcagctgtgttttgggaaaggtgcaacgagctccaggacatagagaagattatggctcagattgaaaggggagaggcgagaattcaaagaagaataagcatcaagaaagcacttgacacaaagattggacggtacaaagcaccttttcatcagctgagaatatcatatggtactaacaaaggaaaaaactatactgaagaagaagatcgttttctgatttgtatgcttcacaaacttggatttgacaaagaaaatgtttatgatgaattgcgacagtgtattcgcaactctcctcagttcagatttgactggtttcttaagtccagaactgcaatggagctccagaggagatgtaataccttaattactttgattgaaagagaaaacatggaactagaagaaaaggagaaggcagagaaaaagaaacgaggaccaaagccttcaacacagaaacgtaaaatggatggcgcacctgatggtcgaggaagaaaaaagaagctgaaactatgaatatgtttttgtttcataatcactaactttaaaccagtagttctttaatttacgggtcttcataagatgtactgtacaatgctcaattgttatgtcatttaaagacatcaggttcatctgtttactgagctagaaacatagtatgtagtttcacttttttaaatgcaacagctgtgctgaaatttttttatcattaacacttgaagtaataaaataggcttcatttattaaaaaaaaaaaaaaaa

SEQ ID NO: 12 - DNA, 3786 bp, Artificial Sequence
  Synthetic
ggcgggcggcggcggggcccgagccggagaagatggcggtgcggaagaaggacggcggccccaacgtgaagtactacgaggccgcggacaccgtgacccagttcgacaacgtgcggctgtggctcggcaagaactacaagaagtatatacaagctgaaccacccaccaacaagtccctgtctagcctggttgtacagttgctacaatttcaggaagaagtttttggcaaacatgtcagcaatgcaccgctcactaaactgccgatcaaatgtttcctagatttcaaagcgggaggctccttgtgccacattcttgcagctgcctacaaattcaagagtgaccagggatggcggcgttacgatttccagaatccatcacgcatggaccgcaatgtggaaatgtttatgaccattgagaagtccttggtgcagaataattgcctgtctcgacctaacatttttctgtgcccagaaattgagcccaaactactagggaaattaaaggacattatcaagagacaccagggaacagtcactgaggataagaacaatgcctcccatgttgtgtatcctgtcccggggaatctagaagaagaggaatgggtacgaccagtcatgaagagggataagcaggttcttctgcactggggctactatcctgacagttacgacacgtggatcccagcgagtgaaattgaggcatctgtggaagatgctccaactcctgagaaacctaggaaggttcatgcaaagtggatcctggacaccgacaccttcaatgaatggatgaatgaggaagactatgaagtaaatgatgacaaaaaccctgtctcccgccgaaagaagatttcagccaagacactgacagatgaggtgaacagcccagattcagatcgacgggacaagaaggggggaaactataagaagaggaagcgctccccctctccttcaccaaccccagaagcaaagaagaaaaatgctaagaaaggtccctcaacaccttacactaagtcaaagcgtggccacagagaagaggagcaagaagacctgacaaaggacatggacgagccctcaccagtccccaatgtagaagaggtgacacttcccaaaacagtcaacacaaagaaagactcagagtcggccccagtcaaaggcggcaccatgaccgacctggatgaacaggaagatgaaagcatggagacgacgggcaaggatgaggatgagaacagtacggggaacaagggagagcagaccaagaatccagacctgcatgaggacaatgtgactgaacagacccaccacatcatcattcccagctacgctgcctggtttgactacaatagtgttcatgccattgagcggagggctctccccgagttcttcaacggcaagaacaagtccaagactccagagatctacctggcctatcgaaactttatgattgacacttaccgactgaacccccaagagtatcttacctctaccgcctgccgccgaaacctagcgggtgatgtctgtgccatcatgagggtccatgccttcctagaacagtggggtcttattaactaccaggtggatgctgagagtcgaccaaccccaatggggcctccgcctacctctcacttccatgtcttggctgacacaccatcagggctggtgcctctgcagcccaagacacctcagggccgccaggttgatgctgataccaaggctgggcgaaagggcaaagagctggatgacctggtgccagagacggctaagggcaagccagagctgcagacctctgcttcccaacaaatgctcaactttcctgacaaaggcaaagagaaaccaacagacatgcaaaactttgggctgcgcacagacatgtacacaaaaaagaatgttccctccaagagcaaggctgcagccagtgccactcgtgagtggacagaacaggaaaccctgcttctcctggaggcactggaaatgtacaaagatgactggaacaaagtgtccgagcatgtgggaagccgcacacaggacgagtgcatcttgcattttcttcgtcttcccattgaagacccatacctggaggactcagaggcctccctaggccccctggcctaccaacccatccccttcagtcagtcgggcaaccctgttatgagcactgttgccttcctggcctctgtcgtcgatccccgagtcgcctctgctgctgcaaagtcagccctagaggagttctccaaaatgaaggaagaggtacccacggccttggtggaggcccatgttcgaaaagtggaagaagcagccaaagtaacaggcaaggcggaccctgccttcggtctggaaagcagtggcattgcaggaaccacctctgatgagcctgagcggattgaggagagcgggaatgacgaggctcgggtggaaggccaggccacagatgagaagaaggagcccaaggaaccccgagaaggagggggtgctatagaggaggaagcaaaagagaaaaccagcgaggctcccaagaaggatgaggagaaagggaaagaaggcgacagtgagaaggagtccgagaagagtgatggagacccaatagtcgatcctgagaaggagaaggagccaaaggaagggcaggaggaagtgctgaaggaagtggtggagtctgagggggaaaggaagacaaaggtggagcgggacattggcgagggcaacctctccaccgctgctgccgccgccctggccgccgccgcagtgaaagctaagcacttggctgctgttgaggaaaggaagatcaaatctttggtggccctgctggtggagacccagatgaaaaagttggagatcaaacttcggcactttgaggagctggagactatcatggaccgggagcgagaagcactggagtatcagaggcagcagctcctggccgacagacaagccttccacatggagcagctgaagtatgcggagatgagggctcggcagcagcacttccaacagatgcaccaacagcagcagcagccaccaccagccctgcccccaggctcccagcctatccccccaacaggggctgctgggccacccgcagtccatggcttggctgtggctccagcctctgtagtccctgctcctgctggcagtggggcccctccaggaagtttgggcccttctgaacagattgggcaggcagggtcaactgcagggccacagcagcagcaaccagctggagccccccagcctggggcagtcccaccaggggttcccccccctggaccccatggcccctcaccgttccccaaccaacaaactcctccctcaatgatgccaggggcagtgccaggcagcgggcacccaggcgtggcggacccaggcacccccctgcctccagaccccacagccccgagcccaggcacggtcacccctgtgccacctccacagtgaggagccagccagacatctctccccctcaccccctgtggacatcacggttccaggaacagcccttcccccaccactgggaccctccccagcctggagagttcatcactacgtaaggaaagctccttccgcccctccaaagccctcaccatgcctaacagaggcatgcatttttatatcagattattcaaggacttctgtttaaaagatgtttataatgtctgggagagaggataggatgggaatgctgccctaaaggaagggctggtgaaaggtgtttatacaaggttctattaaccacttctaagggtacacctccctccaaactactgcattttctatggattaaaaaaaaaaaaaaaaa

SEQ ID NO: 13 - DNA, 7088 bp, Artificial Sequence
  Synthetic
agcgacggcggcggctgcggcttagtcggtggcggccggcggcggctgcgggctgagcggcgagtttccgatttaaagctgagctgcgaggaaaatggcggcgggaggatcaaaatacttgctggatggtggactcagagaccaataaaaataaactgcttgaacatcctttgactggttagccagttgctgatgtatattcaagatgagtggattaggagaaaacttggatccactggccagtgattcacgaaaacgcaaattgccatgtgatactccaggacaaggtcttacctgcagtggtgaaaaacggagacgggagcaggaaagtaaatatattgaagaattggctgagctgatatctgccaatcttagtgatattgacaatttcaatgtcaaaccagataaatgtgcgattttaaaggaaacagtaagacagatacgtcaaataaaagagcaaggaaaaactatttccaatgatgatgatgttcaaaaagccgatgtatcttctacagggcagggagttattgataaagactccttaggaccgcttttacttcaggcattggatggtttcctatttgtggtgaatcgagacggaaacattgtatttgtatcagaaaatgtcacacaatacctgcaatataagcaagaggacctggttaacacaagtgtttacaatatcttacatgaagaagacagaaaggattttcttaagaatttaccaaaatctacagttaatggagtttcctggacaaatgagacccaaagacaaaaaagccatacatttaattgccgtatgttgatgaaaacaccacatgatattctggaagacataaacgccagtcctgaaatgcgccagagatatgaaacaatgcagtgctttgccctgtctcagccacgagctatgatggaggaaggggaagatttgcaatcttgtatgatctgtgtggcacgccgcattactacaggagaaagaacatttccatcaaaccctgagagctttattaccagacatgatctttcaggaaaggttgtcaatatagatacaaattcactgagatcctccatgaggcctggctttgaagatataatccgaaggtgtattcagagattttttagtctaaatgatgggcagtcatggtcccagaaacgtcactatcaagaagcttatcttaatggccatgcagaaaccccagtatatcgattctcgttggctgatggaactatagtgactgcacagacaaaaagcaaactcttccgaaatcctgtaacaaatgatcgacatggctttgtctcaacccacttccttcagagagaacagaatggatatagaccaaacccaaatcctgttggacaagggattagaccacctatggctggatgcaacagttcggtaggcggcatgagtatgtcgccaaaccaaggcttacagatgccgagcagcagggcctatggcttggcagaccctagcaccacagggcagatgagtggagctaggtatgggggttccagtaacatagcttcattgacccctgggccaggcatgcaatcaccatcttcctaccagaacaacaactatgggctcaacatgagtagccccccacatgggagtcctggtcttgccccaaaccagcagaatatcatgatttctcctcgtaatcgtgggagtccaaagatagcctcacatcagttttctcctgttgcaggtgtgcactctcccatggcatcttctggcaatactgggaaccacagcttttccagcagctctctcagtgccctgcaagccatcagtgaaggtgtggggacttcccttttatctactctgtcatcaccaggccccaaattggataactctcccaatatgaatattacccaaccaagtaaagtaagcaatcaggattccaagagtcctctgggcttttattgcgaccaaaatccagtggagagttcaatgtgtcagtcaaatagcagagatcacctcagtgacaaagaaagtaaggagagcagtgttgagggggcagagaatcaaaggggtcctttggaaagcaaaggtcataaaaaattactgcagttacttacctgttcttctgatgaccggggtcattcctccttgaccaactcccccctagattcaagttgtaaagaatcttctgttagtgtcaccagcccctctggagtctcctcctctacatctggaggagtatcctctacatccaatatgcatgggtcactgttacaagagaagcaccggattttgcacaagttgctgcagaatgggaattcaccagctgaggtagccaagattactgcagaagccactgggaaagacaccagcagtataacttcttgtggggacggaaatgttgtcaagcaggagcagctaagtcctaagaagaaggagaataatgcacttcttagatacctgctggacagggatgatcctagtgatgcactctctaaagaactacagccccaagtggaaggagtggataataaaatgagtcagtgcaccagctccaccattcctagctcaagtcaagagaaagaccctaaaattaagacagagacaagtgaagagggatctggagacttggataatctagatgctattcttggtgatctgactagttctgacttttacaataattccatatcctcaaatggtagtcatctggggactaagcaacaggtgtttcaaggaactaattctctgggtttgaaaagttcacagtctgtgcagtctattcgtcctccatataaccgagcagtgtctctggatagccctgtttctgttggctcaagtcctccagtaaaaaatatcagtgctttccccatgttaccaaagcaacccatgttgggtgggaatccaagaatgatggatagtcaggaaaattatggctcaagtatgggtgggccaaaccgaaatgtgactgtgactcagactccttcctcaggagactggggcttaccaaactcaaaggccggcagaatggaacctatgaattcaaactccatgggaagaccaggaggagattataatacttctttacccagacctgcactgggtggctctattcccacattgcctcttcggtctaatagcataccaggtgcgagaccagtattgcagcagcagcagcagcagcagcaacagcaacagcaacagcaacagcagcaacagcagcaaacccaggccttcagcccacctcctaatgtgactgcttcccccagcatggatgggcttttggcaggacccacaatgccacaagctcctccgcaacagtttccatatcaaccaaattatggaatggacaaccagatccagcctttggtcgagtgtctagtcctcccaatgcaatgatgtcgtcaagaatgggtccctcccagaatcccatgatgcaacacccgcaggctgcatccatctatcagtcctcagaaatgaagggctggccatcaggaaatttggccaggaacagctccttttcccagcagcagtttgcccaccaggggaatcctgcagtgtatagtatggtgcacatgaatggcagcagtggtcacatgggacagatgaacatgaaccccatgcccatgtctggcatgcctatgggtcctgatcagaaatactgctgacatctctgcaccaggacctcttaaggaaaccactgtacaaatgacactgcactaggattattgggaaggaatcattgttccaggcatccatcttggaagaaaggaccagctttgagctccatcaagggtattttaagtgatgtcatttgagcaggactggattttaagccgaagggcaatatctacgtgtttttcccccctccttctgctgtgtatcatggtgttcaaaacagaaatgttttttggcattccacctcctagggatataattctggagacatggagtgttactgatcataaaacttttgtgtcacttttttctgccttgctagccaaaatctcttaaatacacgtaggtgggccagagaacattggaagaatcaagagagattagaatatctggtttctctagttgcagtattggacaaagagcatagtcccagccttcaggtgtagtagttctgtgttgaccctttgtccagtggaattggtgattctgaattgtcctttactaatggtgttgagttgctctgtccctattatttgccctaggctttctcctaatgaaggttttcatttgccattcatgtcctgtaatacttcacctccaggaactgtcatggatgtccaaatggctttgcagaaaggaaatgagatgacagtatttaatcgcagcagtagcaaacttttcacatgctaatgtgcagctgagtgcactttatttaaaaagaatggataaatgcaatattcttgaggtcttgagggaatagtgaaacacattcctggtttttgcctacacttacgtgttagacaagaactatgatttttttttttaaagtactggtgtcaccctttgcctatatggtagagcaataatgctttttaaaaataaacttctgaaaacccaaggccaggtactgcattctgaatcagaatctcgcagtgtttctgtgaatagatttttttgtaaatatgacctttaagatattgtattatgtaaaatatgtatatacctttttttgtaggtcacaacaactcatttttacagagtttgtgaagctaaatatttaacattgttgatttcagtaagctgtgtggtgaggctaccagtggaagagacatcccttgacttttgtggcctgggggaggggtagtgctccacagcttttccttccccaccccccagccttagatgcctcgctcttttcaatctcttaatctaaatgctttttaaagagattatttgtttagatgtaggcattttaattttttaaaaattcctctaccagaactaagcactttgttaatttggggggaaagaatagatatggggaaataaacttaaaaaaaaatcaggaatttaaaaaaacgagcaatttgaagagaatcttttggattttaagcagtccgaaataatagcaattcatgggctgtgtgtgtgtgtgtatgtgtgtgtgtgtgtgtgtatgtttaattatgttaccttttcatcccctttaggagcgttttcagattttggttgctaagacctgaatcccatattgagatctcgagtagaatccttggtgtggtttctggtgtctgctcagctgtcccctcattctactaatgtgatgctttcattatgtccctgtggattagaatagtgtcagttatttcttaagtaactcagtacccagaacagccagttttactgtgattcagagccacagtctaactgagcaccttttaaacccctccctcttctgccccctaccacttttctgctgttgcctctctttgacacctgttttagtcagttgggaggaagggaaaaatcaagtttaattccctttatctgggttaattcatttggttcaaatagttgacggaattgggtttctgaatgtctgtgaatttcagaggtctctgctagccttggtatcattttctagcaataactgagagccagttaattttaagaatttcacacatttagccaatctttctagatgtctctgaaggtaagatcatttaatatctttgatatgcttacgagtaagtgaatcctgattatttccagacccaccaccagagtggatcttattttcaaagcagtatagacaattatgagtttgccctctttcccctaccaagttcaaaatatatctaagaaagattgtaaatccgaaaacttccattgtagtggcctgtgcttttcagatagtatactctcctgtttggagacagaggaagaaccaggtcagtctgtctctttttcagctcaattgtatctgacccttctttaagttatgtgtgtggggagaaatagaatggtgctcttatctttcttgactttaaaaaaattattaaaaacaaaaaaaaaataaatttttttgcaatcctttcctcagacctggctccaggctaactggaaggcagcactcccttttttatatagtagaaaaatgaagtttattataagtttttatattttctacttgttcatttggtgcaaactcaagatttcttttaataggtgcagtctttgagataatttgtttttacctgtattgccctttatcttttttaggtaattctttgtactcctgctgtctacctctcctcacaccccagcaccccccattttttcaaaccttggtatctgttgggtgaacagtataatcttttcatctgcttttagaatgtgggatatttccagtacctactttttttttttttttttgctgaatccaaagatatataaataaaatatatatattttataaagatcagaatgatataaaggagatacatgtttcttcctttaaaaaataaacggaagttacattgttaatgttcatattatgatgccacttttctaaactgcatctggattgaaaggtgtaaatatcaataacagtgctacttagttatcagtatttaatatctgaggtgagttgggggtatctatattaggggtagggtattacagaagataattggcttgatgtcctagaagttctttgatccagaggtgggtgcagctgaaagtaaacagaatggattgccagttacatgtatgcctgcccagttccctttttatttgcagaagctgtgagttttgttcacaattaggttcctaggagcaaaacctcaaggattgatttattgttttcaactccaaggcacactgttaataaacgagcagggtgttttctctcttcctttctaatatatggagtttcgaagaataaaatatgagagcaatatttaaattctcaggaattgacttatactcttgagaatgaattcagtttcaatcaagtttacattatgttgcttaaaaaaatagaaattattctttatcttgcaaagaattgaaaccacatgaaatgacttatgggggatggtgagctgtgactgctttgctgaccattttggatgtcattgtaaataaaggtttctatttaaaa

SEQ ID NO: 14 - DNA, 3525 bp, Artificial Sequence
  Synthetic
aatggcgatgcctaccacctagaactggattgtgcgctggccgccaccgctgccacctgctcagagtgaaataatgaaggtggtcaacctgaagcaagccattttgcaagcctggaaggagcgctggagttactaccaatgggcaatcaacatgaagaaattctttcctaaaggagccacctgggatattctcaacctggcagatgcgttactagagcaggccatgattggaccatcccccaatcctctcatcttgtcctacctgaagtatgccattagttcccagatggtgtcctactcttctgtcctcacagccatcagtaagtttgatgacttttctcgggacctgtgtgtccaggcattgctggacatcatggacatgttttgtgaccgtctgagctgtcacggcaaagcagaggaatgcatcggactgtgccgagcccttcttagcgccctccactggctgctgcgctgcacggcagcctctgcagagcggctgcgggaggggctggaggccggcactccagccgctggggagaagcagcttgccatgtgccttcagcgcctggagaaaaccctcagcagcaccaagaaccgggccctgctgcacatcgccaaactagaggaggcctcattgcacacatcccagggacttgggcagggtggcacccgagccaatcaaccaacagcttcttggactgccatcgagcattctctcttgaaacttggagagatcctgaccaatctcagcaacccgcagctccggagtcaggccgagcagtgtggcaccctcattaggagcatccccacgatgctgtctgtgcatgcggagcagatgcacaagaccggcttccccactgtccacgccgtgatcctgctcgagggcaccatgaacctgacaggcgagacgcagtccctggtggagcagctgacgatggtgaagcgcatgcagcatatccccaccccactttttgtcctggagatctggaaagcttgcttcgtggggctcattgagtctcccgagggtacggaggagctcaagtggacagctttcactttcctcaagattccacaggttttggtgaagttgaagaagtactctcatggagacaaggacttcactgaggatgtcaactgtgcttttgagttcctgctgaagctcacccccttgttggacaaagctgaccagcgctgcaactgtgactgtacaaacttcctgctccaagaatgtggcaagcaggggcttctgtctgaggccagcgtcaacaaccttatggctaagcgcaaagcggaccgagagcacgcaccccagcagaaatcgggagagaatgccaacatccagcccaacatccagctgatcctccgggcggagcccactgtcacaaacatcctcaagacgatggatgcagaccactctaagtcaccggagggactgctgggagtcctgggccacatgctgtccgggaagagtctggacttgctgctggctgccgccgccgccactggaaagctgaaatccttcgcccggaaattcatcaatttgaatgaattcacaacctatggcagcgaagaaagcaccaaaccggcctccgtccgggccctgctgtttgacatctccttcctcatgctgtgccatgtggcccagacctatggttcagaggtgattctgtccgagtcgcgcacaggagctgaggtgcccttcttcgagacctggatgcagacctgcatgcctgaggagggcaagatcctgaaccctgaccacccctgcttccgccccgactccaccaaagtggagtccctggtggccctgctcaacaactcctcggagatgaagctagtgcagatgaagtggcatgaggcctgtctcagcatctcagccgccatcttggaaatcctcaatgcctgggagaatggggtcctggccttcgagtccatccagaaaatcactgataacatcaaagggaaggtatgcagtctggcggtgtgtgctgtggcttggcttgtggcccacgtccggatgctggggctggatgagcgtgagaagtcgctgcagatgatccgccagctggcagggccactgtttagtgagaacaccctgcagttctacaatgagagggtggtgatcatgaactcgatcctggagcgcatgtgtgccgacgtgctgcagcagacagccacgcagatcaagtttccctccaccggggtggacacaatgccctactggaacctgctgccccccaagcggcccatcaaagaggtgctgacggacatttttgccaaggtgctggagaagggctgggtggacagccgctccatccacatctttgacaccctgctgcacatgggcggcgtctactggttctgcaacaacctgattaaggagctgctgaaggagacgcggaaggagcacacgctgcgggcagtggagctgctctactccatcttctgcctggacatgcagcaagtgaccctggtcctgctgggccacatcctacctggcctgctcactgactcctccaagtggcacagcctcatggaccccccgggcactgctcttgccaagctggccgtgtggtgtgccctcagttcctactcctcccacaagggacaggcgtccacccgccagaagaagagacaccgcgaagacattgaggattatatcagcctcttccccctggacgatgtgcagccttcgaagttgatgcgactgctgagctctaatgaggacgatgccaacatcctttcgagccccacagaccgatccatgagcagctccctctcagcctctcagctccacacggtcaacatgcgggaccctctgaaccgagtcctggccaacctgttcctgctcatctcctccatcctggggtctcgcaccgctggcccccacacccagttcgtgcagtggttcatggaggagtgtgtggactgcctggagcagggtggccgtggcagcgtcctgcagttcatgcccttcaccaccgtgtcggaactggtgaaggtgtcagccatgtccagccccaaggtggttctggccatcacggacctcagcctgcccctgggccgccaggtggctgctaaagccattgctgcactctgaggggcttggcatggccgcagtgggggctggggactggcgcagccccaggcgcctccaagggaagcagtgaggaaagatgaggcatcgtgcctcacatccgctccacatggtgcaagagcctctagcggcttccagttccccgctcctgactcctgacctccaggatgtctcccggtttcttctttcaaaatttcctctccatctgctggcacctgaggagagtgagcagcctggaccacaagcccagtggtcacccctgtgtgcgcccgccccagcccaggagtagtcttacctctgaggaactttctagatgcaaagtgtgtatgtgtgtgtgtgtgtgtgtgtgtgtgtttgtgtgtattttgtaatatgtgagggaaatctaccttcgttcatgtataaataaagctcctcgtggctccctt

SEQ ID NO: 15 - DNA, 3340 bp, Artificial Sequence
  Synthetic
aatggcgatgcctaccacctagaactggattgtgcgctggccgccaccgctgccacctgctcagagtgaaataatgaaggtggtcaacctgaagcaagccattttgcaagcctggaaggagcgctggagttactaccaatgggcaatcaacatgaagaaattctttcctaaaggagccacctgggatattctcaacctggcagatgcgttactagagcaggccatgattggaccatcccccaatcctctcatcttgtcctacctgaagtatgccattagttcccagatggtgtcctactcttctgtcctcacagccatcagtaagtttgatgacttttctcgggacctgtgtgtccaggcattgctggacatcatggacatgttttgtgaccgtctgagctgtcacggcaaagcagcttgccatgtgccttcagcgcctggagaaaaccctcagcagcaccaagaaccgggccctgctgcacatcgccaaactagaggaggcctcttcttggactgccatcgagcattctctcttgaaacttggagagatcctgaccaatctcagcaacccgcagctccggagtcaggccgagcagtgtggcaccctcattaggagcatccccacgatgctgtctgtgcatgcggagcagatgcacaagaccggcttccccactgtccacgccgtgatcctgctcgagggcaccatgaacctgacaggcgagacgcagtccctggtggagcagctgacgatggtgaagcgcatgcagcatatccccaccccactttttgtcctggagatctggaaagcttgcttcgtggggctcattgagtctcccgagggtacggaggagctcaagtggacagctttcactttcctcaagattccacaggttttggtgaagttgaagaagtactctcatggagacaaggacttcactgaggatgtcaactgtgcttttgagttcctgctgaagctcacccccttgttggacaaagctgaccagcgctgcaactgtgactgtacaaacttcctgctccaagaatgtggcaagcaggggcttctgtctgaggccagcgtcaacaaccttatggctaagcgcaaagcggaccgagagcacgcaccccagcagaaatcgggagagaatgccaacatccagcccaacatccagctgatcctccgggcggagcccactgtcacaaacatcctcaagacgatggatgcagaccactctaagtcaccggagggactgctgggagtcctgggccacatgctgtccgggaagagtctggacttgctgctggctgccgccgccgccactggaaagctgaaatccttcgcccggaaattcatcaatttgaatgaattcacaacctatggcagcgaagaaagcaccaaaccggcctccgtccgggccctgctgtttgacatctccttcctcatgctgtgccatgtggcccagacctatggttcagaggtgattctgtccgagtcgcgcacaggagctgaggtgcccttcttcgagacctggatgcagacctgcatgcctgaggagggcaagatcctgaaccctgaccacccctgcttccgccccgactccaccaaagtggagtccctggtggccctgctcaacaactcctcggagatgaagctagtgcagatgaagtggcatgaggcctgtctcagcatctcagccgccatcttggaaatcctcaatgcctgggagaatggggtcctggccttcgagtccatccagaaaatcactgataacatcaaagggaaggtatgcagtctggcggtgtgtgctgtggcttggcttgtggcccacgtccggatgctggggctggatgagcgtgagaagtcgctgcagatgatccgccagctggcagggccactgtttagtgagaacaccctgcagttctacaatgagagggtggtgatcatgaactcgatcctggagcgcatgtgtgccgacgtgctgcagcagacagccacgcagatcaagtttccctccaccggggtggacacaatgccctactggaacctgctgccccccaagcggcccatcaaagaggtgctgacggacatttttgccaaggtgctggagaagggctgggtggacagccgctccatccacatctttgacaccctgctgcacatgggcggcgtctactggttctgcaacaacctgattaaggagctgctgaaggagacgcggaaggagcacacgctgcgggcagtggagctgctctactccatcttctgcctggacatgcagcaagtgaccctggtcctgctgggccacatcctacctggcctgctcactgactcctccaagtggcacagcctcatggaccccccgggcactgctcttgccaagctggccgtgtggtgtgccctcagttcctactcctcccacaagggacaggcgtccacccgccagaagaagagacaccgcgaagacattgaggattatatcagcctcttccccctggacgatgtgcagccttcgaagttgatgcgactgctgagctctaatgaggacgatgccaacatcctttcgagccccacagaccgatccatgagcagctccctctcagcctctcagctccacacggtcaacatgcgggaccctctgaaccgagtcctggccaacctgttcctgctcatctcctccatcctggggtctcgcaccgctggcccccacacccagttcgtgcagtggttcatggaggagtgtgtggactgcctggagcagggtggccgtggcagcgtcctgcagttcatgcccttcaccaccgtgtcggaactggtgaaggtgtcagccatgtccagccccaaggtggttctggccatcacggacctcagcctgcccctgggccgccaggtggctgctaaagccattgctgcactctgaggggcttggcatggccgcagtgggggctggggactggcgcagccccaggcgcctccaagggaagcagtgaggaaagatgaggcatcgtgcctcacatccgctccacatggtgcaagagcctctagcggcttccagttccccgctcctgactcctgacctccaggatgtctcccggtttcttctttcaaaatttcctctccatctgctggcacctgaggagagtgagcagcctggaccacaagcccagtggtcacccctgtgtgcgcccgccccagcccaggagtagtcttacctctgaggaactttctagatgcaaagtgtgtatgtgtgtgtgtgtgtgtgtgtgtgtgtttgtgtgtattttgtaatatgtgagggaaatctaccttcgttcatgtataaataaagctcctcgtggctccctt

SEQ ID NO: 16 - DNA, 3548 bp, Artificial Sequence
  Synthetic
aatggcgatgcctaccacctagaactggattgtgcgctggccgccaccgctgccacctgctcagagtgaaataatgaaggtggtcaacctgaagcaagccattttgcaagcctggaaggagcgctggagttactaccaatgggcaatcaacatgaagaaattctttcctaaaggagccacctgggatattctcaacctggcagatgcgttactagagcaggccatgattggaccatcccccaatcctctcatcttgtcctacctgaagtatgccattagttcccagatggtgtcctactcttctgtcctcacagccatcagtaagcatctccactgtcctttccacagtttgatgacttttctcgggacctgtgtgtccaggcattgctggacatcatggacatgttttgtgaccgtctgagctgtcacggcaaagcagaggaatgcatcggactgtgccgagcccttcttagcgccctccactggctgctgcgctgcacggcagcctctgcagagcggctgcgggaggggctggaggccggcactccagccgctggggagaagcagcttgccatgtgccttcagcgcctggagaaaaccctcagcagcaccaagaaccgggccctgctgcacatcgccaaactagaggaggcctcattgcacacatcccagggacttgggcagggtggcacccgagccaatcaaccaacagcttcttggactgccatcgagcattctctcttgaaacttggagagatcctgaccaatctcagcaacccgcagctccggagtcaggccgagcagtgtggcaccctcattaggagcatccccacgatgctgtctgtgcatgcggagcagatgcacaagaccggcttccccactgtccacgccgtgatcctgctcgagggcaccatgaacctgacaggcgagacgcagtccctggtggagcagctgacgatggtgaagcgcatgcagcatatccccaccccactttttgtcctggagatctggaaagcttgcttcgtggggctcattgagtctcccgagggtacggaggagctcaagtggacagctttcactttcctcaagattccacaggttttggtgaagttgaagaagtactctcatggagacaaggacttcactgaggatgtcaactgtgcttttgagttcctgctgaagctcacccccttgttggacaaagctgaccagcgctgcaactgtgactgtacaaacttcctgctccaagaatgtggcaagcaggggcttctgtctgaggccagcgtcaacaaccttatggctaagcgcaaagcggaccgagagcacgcaccccagcagaaatcgggagagaatgccaacatccagcccaacatccagctgatcctccgggcggagcccactgtcacaaacatcctcaagacgatggatgcagaccactctaagtcaccggagggactgctgggagtcctgggccacatgctgtccgggaagagtctggacttgctgctggctgccgccgccgccactggaaagctgaaatccttcgcccggaaattcatcaatttgaatgaattcacaacctatggcagcgaagaaagcaccaaaccggcctccgtccgggccctgctgtttgacatctccttcctcatgctgtgccatgtggcccagacctatggttcagaggtgattctgtccgagtcgcgcacaggagctgaggtgcccttcttcgagacctggatgcagacctgcatgcctgaggagggcaagatcctgaaccctgaccacccctgcttccgccccgactccaccaaagtggagtccctggtggccctgctcaacaactcctcggagatgaagctagtgcagatgaagtggcatgaggcctgtctcagcatctcagccgccatcttggaaatcctcaatgcctgggagaatggggtcctggccttcgagtccatccagaaaatcactgataacatcaaagggaaggtatgcagtctggcggtgtgtgctgtggcttggcttgtggcccacgtccggatgctggggctggatgagcgtgagaagtcgctgcagatgatccgccagctggcagggccactgtttagtgagaacaccctgcagttctacaatgagagggtggtgatcatgaactcgatcctggagcgcatgtgtgccgacgtgctgcagcagacagccacgcagatcaagtttccctccaccggggtggacacaatgccctactggaacctgctgccccccaagcggcccatcaaagaggtgctgacggacatttttgccaaggtgctggagaagggctgggtggacagccgctccatccacatctttgacaccctgctgcacatgggcggcgtctactggttctgcaacaacctgattaaggagctgctgaaggagacgcggaaggagcacacgctgcgggcagtggagctgctctactccatcttctgcctggacatgcagcaagtgaccctggtcctgctgggccacatcctacctggcctgctcactgactcctccaagtggcacagcctcatggaccccccgggcactgctcttgccaagctggccgtgtggtgtgccctcagttcctactcctcccacaagggacaggcgtccacccgccagaagaagagacaccgcgaagacattgaggattatatcagcctcttccccctggacgatgtgcagccttcgaagttgatgcgactgctgagctctaatgaggacgatgccaacatcctttcgagccccacagaccgatccatgagcagctccctctcagcctctcagctccacacggtcaacatgcgggaccctctgaaccgagtcctggccaacctgttcctgctcatctcctccatcctggggtctcgcaccgctggcccccacacccagttcgtgcagtggttcatggaggagtgtgtggactgcctggagcagggtggccgtggcagcgtcctgcagttcatgcccttcaccaccgtgtcggaactggtgaaggtgtcagccatgtccagccccaaggtggttctggccatcacggacctcagcctgcccctgggccgccaggtggctgctaaagccattgctgcactctgaggggcttggcatggccgcagtgggggctggggactggcgcagccccaggcgcctccaagggaagcagtgaggaaagatgaggcatcgtgcctcacatccgctccacatggtgcaagagcctctagcggcttccagttccccgctcctgactcctgacctccaggatgtctcccggtttcttctttcaaaatttcctctccatctgctggcacctgaggagagtgagcagcctggaccacaagcccagtggtcacccctgtgtgcgcccgccccagcccaggagtagtcttacctctgaggaactttctagatgcaaagtgtgtatgtgtgtgtgtgtgtgtgtgtgtgtgtttgtgtgtattttgtaatatgtgagggaaatctaccttcgttcatgtataaataaagctcctcgtggctccctt

SEQ ID NO: 17 - DNA, 21 bp, Artificial Sequence
  Synthetic
ttggttcccttgtgttgattc

SEQ ID NO: 18 - DNA, 21 bp, Artificial Sequence
  Synthetic
tggaaaccaacatctgactcc

SEQ ID NO: 19 - DNA, 21 bp, Artificial Sequence
  Synthetic
gaccaacatccagaacttcca

SEQ ID NO: 20 - DNA, 20 bp, Artificial Sequence
  Synthetic
tgctttgagagcagcagtga

SEQ ID NO: 21 - DNA, 21 bp, Artificial Sequence
  Synthetic
agacatgagtgaaagccagga

SEQ ID NO: 22 - DNA, 21 bp, Artificial Sequence
  Synthetic
cataaggcaactgaagggaca

SEQ ID NO: 23 - DNA, 21 bp, Artificial Sequence
  Synthetic
ggccatatctaacggggttta

SEQ ID NO: 24 - DNA, 21 bp, Artificial Sequence
  Synthetic
gggacatggggacagataagt

SEQ ID NO: 25 - DNA, 21 bp, Artificial Sequence
  Synthetic
ttgatgaccctccttcagcta

SEQ ID NO: 26 - DNA, 21 bp, Artificial Sequence
  Synthetic
gcaaaactctggcaatttcac

SEQ ID NO: 27 - DNA, 21 bp, Artificial Sequence
  Synthetic
agatgactcgcttgctggata

SEQ ID NO: 28 - DNA, 21 bp, Artificial Sequence
  Synthetic
aggttaattccgagacctcca

SEQ ID NO: 29 - DNA, 21 bp, Artificial Sequence
  Synthetic
ctgaggctctgtaccgtgaac

SEQ ID NO: 30 - DNA, 21 bp, Artificial Sequence
  Synthetic
tgaaatccactggcttcctaa

SEQ ID NO: 31 - DNA, 21 bp, Artificial Sequence
  Synthetic
accacaaagtgctgctgttct

SEQ ID NO: 32 - DNA, 21 bp, Artificial Sequence
  Synthetic
ttcttctgcttcttgctctcg

SEQ ID NO: 33 - DNA, 18 bp, Artificial Sequence
  Synthetic
atttcgccttccggcttc

SEQ ID NO: 34 - DNA, 21 bp, Artificial Sequence
  Synthetic
tacttctcatcgttgccatcc

SEQ ID NO: 35 - DNA, 21 bp, Artificial Sequence
  Synthetic
ctgctgttgaggaaaggaaga

SEQ ID NO: 36 - DNA, 19 bp, Artificial Sequence
  Synthetic
agatgtctggctggctcct

SEQ ID NO: 37 - DNA, 21 bp, Artificial Sequence
  Synthetic
aaccccagaagcaaagaagaa

SEQ ID NO: 38 - DNA, 21 bp, Artificial Sequence
  Synthetic
cggacactttgttccagtcat

SEQ ID NO: 39 - DNA, 21 bp, Artificial Sequence
  Synthetic
atgactctccaggtgcaggac

SEQ ID NO: 40 - DNA, 21 bp, Artificial Sequence
  Synthetic
acttttaatccagccccacac

SEQ ID NO: 41 - DNA, 21 bp, Artificial Sequence
  Synthetic
tagccagctctttgtcggata

SEQ ID NO: 42 - DNA, 21 bp, Artificial Sequence
  Synthetic
aggagagctccctcatcactc

SEQ ID NO: 43 - DNA, 21 bp, Artificial Sequence
  Synthetic
gagggacttggagcttgctat

SEQ ID NO: 44 - DNA, 21 bp, Artificial Sequence
  Synthetic
ggtcagacccagaaacacaaa

SEQ ID NO: 45 - DNA, 21 bp, Artificial Sequence
  Synthetic
gccacctcaaaataacccact

SEQ ID NO: 46 - DNA, 21 bp, Artificial Sequence
  Synthetic
ggttctgagggttcaaggttc

SEQ ID NO: 47 - DNA, 22 bp, Artificial Sequence
  Synthetic
gagaagaaggaacggaaacaaa

SEQ ID NO: 48 - DNA, 21 bp, Artificial Sequence
  Synthetic
caatggaaacaacctcttcca

SEQ ID NO: 49 - DNA, 23 bp, Artificial Sequence
  Synthetic
agtggtgcgtgatgtggctaaga

SEQ ID NO: 50 - DNA, 23 bp, Artificial Sequence
  Synthetic
gcttgaagtcctcctcctcctct

SEQ ID NO: 51 - DNA, 22 bp, Artificial Sequence
  Synthetic
ggtcatcagtgtggtcaaagtg

SEQ ID NO: 52 - DNA, 24 bp, Artificial Sequence
  Synthetic
gctgagacctcctacgagtggtac

SEQ ID NO: 53 - DNA, 21 bp, Artificial Sequence
  Synthetic
cgtcctactacatcttcaccc

SEQ ID NO: 54 - DNA, 21 bp, Artificial Sequence
  Synthetic
ctcttgggtggcgtcttcttc

SEQ ID NO: 55 - DNA, 20 bp, Artificial Sequence
  Synthetic
gaggaggattgcatccaggt

SEQ ID NO: 56 - DNA, 20 bp, Artificial Sequence
  Synthetic
tcctccaccaacctcttcag

SEQ ID NO: 57 - DNA, 21 bp, Artificial Sequence
  Synthetic
cccagccagcagactacaatg

SEQ ID NO: 58 - DNA, 21 bp, Artificial Sequence
  Synthetic
ctaatgcccatgtgctctctg

SEQ ID NO: 59 - DNA, 19 bp, Artificial Sequence
  Synthetic
atggctggctacgaatacg

SEQ ID NO: 60 - DNA, 20 bp, Artificial Sequence
  Synthetic
gctacgaagttgaggatgcc

SEQ ID NO: 61 - DNA, 20 bp, Artificial Sequence
  Synthetic
gcacctccctttcatctggt

SEQ ID NO: 62 - DNA, 21 bp, Artificial Sequence
  Synthetic
cccactcgactttcctcttag

SEQ ID NO: 63 - DNA, 22 bp, Artificial Sequence
  Synthetic
actaggcagagcagaggagtgg

SEQ ID NO: 64 - DNA, 23 bp, Artificial Sequence
  Synthetic
ctacagagcaggagttgccgcag

SEQ ID NO: 65 - DNA, 26 bp, Artificial Sequence
  Synthetic
gctgcagttactcctttgagacacca

SEQ ID NO: 66 - DNA, 22 bp, Artificial Sequence
  Synthetic
ccaccacttgtatccagcaccc

SEQ ID NO: 67 - DNA, 20 bp, Artificial Sequence
  Synthetic
cgctggtgtgtgatgggtac

SEQ ID NO: 68 - DNA, 22 bp, Artificial Sequence
  Synthetic
gatggggaacctcacttcgtgg

SEQ ID NO: 69 - DNA, 22 bp, Artificial Sequence
  Synthetic
ctcccagcatggacagcatctc

SEQ ID NO: 70 - DNA, 24 bp, Artificial Sequence
  Synthetic
ggggtgtccatcacaaaagccgag

SEQ ID NO: 71 - DNA, 21 bp, Artificial Sequence
  Synthetic
atgggaagggcctgctgaatc

SEQ ID NO: 72 - DNA, 21 bp, Artificial Sequence
  Synthetic
ctgcaaaagggaacaagagcc

SEQ ID NO: 73 - DNA, 21 bp, Artificial Sequence
  Synthetic
agggttctggaaggggatcac

SEQ ID NO: 74 - DNA, 26 bp, Artificial Sequence
  Synthetic
tgccaagtatgaccgctacctcaaca

SEQ ID NO: 75 - DNA, 24 bp, Artificial Sequence
  Synthetic
agaaagcagcgtggaccgagactg

SEQ ID NO: 76 - DNA, 23 bp, Artificial Sequence
  Synthetic
gctattttcaggcacggtttctc

SEQ ID NO: 77 - DNA, 21 bp, Artificial Sequence
  Synthetic
tccacattgttggggtcgttc

SEQ ID NO: 78 - DNA, 23 bp, Artificial Sequence
  Synthetic
gactagaatatcaatgaaccagg

SEQ ID NO: 79 - DNA, 21 bp, Artificial Sequence
  Synthetic
gcagtgccagtaaaaactccc

SEQ ID NO: 80 - DNA, 23 bp, Artificial Sequence
  Synthetic
cctggcggaactggatttctctc

SEQ ID NO: 81 - DNA, 21 bp, Artificial Sequence
  Synthetic
gttggatcattgagctgctgg

SEQ ID NO: 82 - DNA, 21 bp, Artificial Sequence
  Synthetic
ctgtaccctccaatgacatcg

SEQ ID NO: 83 - DNA, 20 bp, Artificial Sequence
  Synthetic
ggaagagcttgccatcagtg

SEQ ID NO: 84 - DNA, 20 bp, Artificial Sequence
  Synthetic
tgccctactacctggagaac

SEQ ID NO: 85 - DNA, 22 bp, Artificial Sequence
  Synthetic
ctgatgtgggagaggatgagga

SEQ ID NO: 86 - DNA, 22 bp, Artificial Sequence
  Synthetic
gctctgttctgttccattggtc

SEQ ID NO: 87 - DNA, 23 bp, Artificial Sequence
  Synthetic
gaagaggcagaagtgtttcaggg

SEQ ID NO: 88 - DNA, 22 bp, Artificial Sequence
  Synthetic
tggaggaactgaaagtgcgatg

SEQ ID NO: 89 - DNA, 21 bp, Artificial Sequence
  Synthetic
tgtcagtaaacgggcaggtac

SEQ ID NO: 90 - DNA, 22 bp, Artificial Sequence
  Synthetic
ctggctacaagaggagaaggac

SEQ ID NO: 91 - DNA, 22 bp, Artificial Sequence
  Synthetic
aacaagcagagcagtgagtcgg

SEQ ID NO: 92 - DNA, 24 bp, Artificial Sequence
  Synthetic
ggcacacaaactccttcttctccc

SEQ ID NO: 93 - DNA, 22 bp, Artificial Sequence
  Synthetic
tgctggcgggcgccgaggtgca

SEQ ID NO: 94 - DNA, 23 bp, Artificial Sequence
  Synthetic
tgctgctggcgggcgccgaggcc

SEQ ID NO: 95 - DNA, 23 bp, Artificial Sequence
  Synthetic
gcatggactcgagcagatggttc

SEQ ID NO: 96 - DNA, 21 bp, Artificial Sequence
  Synthetic
ttcttttggggggaggggaac

SEQ ID NO: 97 - DNA, 20 bp, Artificial Sequence
  Synthetic
gcttttgagaagcagggatc

SEQ ID NO: 98 - DNA, 20 bp, Artificial Sequence
  Synthetic
tgagaagcaggtaatggagc

SEQ ID NO: 99 - DNA, 21 bp, Artificial Sequence
  Synthetic
gctcagataacgcgcaacttc

SEQ ID NO: 100 - DNA, 22 bp, Artificial Sequence
  Synthetic
ctcaattgaccactcgcacacc

SEQ ID NO: 101 - DNA, 21 bp, Artificial Sequence
  Synthetic
gtcctactcccactcaagttg

SEQ ID NO: 102 - DNA, 22 bp, Artificial Sequence
  Synthetic
ctccttctccagttccgtgagc

SEQ ID NO: 103 - DNA, 23 bp, Artificial Sequence
  Synthetic
aaattcctcgtcccccggtcagc

SEQ ID NO: 104 - DNA, 22 bp, Artificial Sequence
  Synthetic
aaattcctcgtccccggtcagc

SEQ ID NO: 105 - DNA, 24 bp, Artificial Sequence
  Synthetic
cggaggtgcttcactgtcatttcc

SEQ ID NO: 106 - DNA, 22 bp, Artificial Sequence
  Synthetic
tggctgggctgctcgggttaga

SEQ ID NO: 107 - DNA, 25 bp, Artificial Sequence
  Synthetic
ctccttctctttcgtctggtcactc

SEQ ID NO: 108 - DNA, 19 bp, Artificial Sequence
  Synthetic
tgctgtaaccacctcacag

SEQ ID NO: 109 - DNA, 23 bp, Artificial Sequence
  Synthetic
cacactctcttctttgtcttggg

SEQ ID NO: 110 - DNA, 22 bp, Artificial Sequence
  Synthetic
gtgcagacccacctcgaaaacc

SEQ ID NO: 111 - DNA, 24 bp, Artificial Sequence
  Synthetic
ccagacattcacaacaagcggaac

SEQ ID NO: 112 - DNA, 22 bp, Artificial Sequence
  Synthetic
ggacgctcgtgaatgtgtgttc

SEQ ID NO: 113 - DNA, 21 bp, Artificial Sequence
  Synthetic
agggggttccaaggaaatggg

SEQ ID NO: 114 - DNA, 22 bp, Artificial Sequence
  Synthetic
tgacctcccttcacacgcttcc

SEQ ID NO: 115 - DNA, 24 bp, Artificial Sequence
  Synthetic
gcctgactttgagggactgtatcc

SEQ ID NO: 116 - DNA, 22 bp, Artificial Sequence
  Synthetic
cctccccttcccatgagaatcc

SEQ ID NO: 117 - DNA, 22 bp, Artificial Sequence
  Synthetic
ggaggagcagcgagtcaagatg

SEQ ID NO: 118 - DNA, 20 bp, Artificial Sequence
  Synthetic
gcctgggctgttgagattgc

SEQ ID NO: 119 - DNA, 20 bp, Artificial Sequence
  Synthetic
ccagctacagcccccatatg

SEQ ID NO: 120 - DNA, 20 bp, Artificial Sequence
  Synthetic
gattcccgctgccatcaagg

SEQ ID NO: 121 - DNA, 26 bp, Artificial Sequence
  Synthetic
agatggttctgctttagtgaagttgg

SEQ ID NO: 122 - DNA, 23 bp, Artificial Sequence
  Synthetic
gtcatcaaacacagcaaaggaag

SEQ ID NO: 123 - DNA, 22 bp, Artificial Sequence
  Synthetic
tttccagcgcctccaatgaccc

SEQ ID NO: 124 - DNA, 22 bp, Artificial Sequence
  Synthetic
gtcggcctgaagcttgatgtgg

SEQ ID NO: 125 - DNA, 19 bp, Artificial Sequence
  Synthetic
aaatggcggacgggaaggc

SEQ ID NO: 126 - DNA, 21 bp, Artificial Sequence
  Synthetic
aaagcggctccaaagatagtc

SEQ ID NO: 127 - DNA, 22 bp, Artificial Sequence
  Synthetic
atggtgtccttacctgtgggag

SEQ ID NO: 128 - DNA, 20 bp, Artificial Sequence
  Synthetic
tacagcatctgcccactgac

SEQ ID NO: 129 - DNA, 23 bp, Artificial Sequence
  Synthetic
gcaaacctctcaccttccaaatc

SEQ ID NO: 130 - DNA, 19 bp, Artificial Sequence
  Synthetic
tggaagcccagagctcgga

SEQ ID NO: 131 - DNA, 23 bp, Artificial Sequence
  Synthetic
caggagaatgaaccagccgcaga

SEQ ID NO: 132 - DNA, 24 bp, Artificial Sequence
  Synthetic
gcaataacttctcgtccagccctt

SEQ ID NO: 133 - DNA, 23 bp, Artificial Sequence
  Synthetic
cctcgtccaggtggtcttctatc

SEQ ID NO: 134 - DNA, 22 bp, Artificial Sequence
  Synthetic
gctgctttgggattcaggttcc

SEQ ID NO: 135 - DNA, 24 bp, Artificial Sequence
  Synthetic
caacaacatcttctgctccaaccc

SEQ ID NO: 136 - DNA, 25 bp, Artificial Sequence
  Synthetic
tcactggactcactgctgctgtcat

SEQ ID NO: 137 - DNA, 23 bp, Artificial Sequence
  Synthetic
cccagcttgaatgcatgacctgg

SEQ ID NO: 138 - DNA, 20 bp, Artificial Sequence
  Synthetic
ttggccaccgacagctgaag

SEQ ID NO: 139 - DNA, 23 bp, Artificial Sequence
  Synthetic
ctgcgaggaatctcaacaaagcc

SEQ ID NO: 140 - DNA, 22 bp, Artificial Sequence
  Synthetic
aggaaggtctccagcaccttgg

SEQ ID NO: 141 - DNA, 23 bp, Artificial Sequence
  Synthetic
atcttggctcactgcaacctccg

SEQ ID NO: 142 - DNA, 20 bp, Artificial Sequence
  Synthetic
tagacagcgcagggccatgg

SEQ ID NO: 143 - DNA, 22 bp, Artificial Sequence
  Synthetic
gtgtgcctcatttgctgctggg

SEQ ID NO: 144 - DNA, 23 bp, Artificial Sequence
  Synthetic
ggcgggtttccagtctgtggctc

SEQ ID NO: 145 - DNA, 23 bp, Artificial Sequence
  Synthetic
ctatccgagaccaggtatgttgc

SEQ ID NO: 146 - DNA, 24 bp, Artificial Sequence
  Synthetic
ctgtaatccagcatcagtaggaca

SEQ ID NO: 147 - DNA, 21 bp, Artificial Sequence
  Synthetic
ccgcccacagatgtagttttc

SEQ ID NO: 148 - DNA, 22 bp, Artificial Sequence
  Synthetic
catcaagtcccccaccaacacc

SEQ ID NO: 149 - DNA, 22 bp, Artificial Sequence
  Synthetic
gccccttattcgctccgacaag

SEQ ID NO: 150 - DNA, 21 bp, Artificial Sequence
  Synthetic
tgtcatctgctgtggctgttc

SEQ ID NO: 151 - DNA, 22 bp, Artificial Sequence
  Synthetic
acgccctttaccacatcccagc

SEQ ID NO: 152 - DNA, 21 bp, Artificial Sequence
  Synthetic
aaaggtataccggagggaggg

SEQ ID NO: 153 - DNA, 24 bp, Artificial Sequence
  Synthetic
ccctggcggcgattactacacttc

SEQ ID NO: 154 - DNA, 22 bp, Artificial Sequence
  Synthetic
ttcctgggacgatggagaaggg

SEQ ID NO: 155 - DNA, 24 bp, Artificial Sequence
  Synthetic
cccaacactgccgagctcaagatc

SEQ ID NO: 156 - DNA, 23 bp, Artificial Sequence
  Synthetic
ccagaaggaaacaccatggtggg

SEQ ID NO: 157 - DNA, 22 bp, Artificial Sequence
  Synthetic
caatcggaagcctaactacagc

SEQ ID NO: 158 - DNA, 22 bp, Artificial Sequence
  Synthetic
ctcggggcatctcagactctag

SEQ ID NO: 159 - DNA, 24 bp, Artificial Sequence
  Synthetic
ccgaggcaaaggcccttttgaagg

SEQ ID NO: 160 - DNA, 20 bp, Artificial Sequence
  Synthetic
agagcagggcagggttcatg

SEQ ID NO: 161 - DNA, 20 bp, Artificial Sequence
  Synthetic
tccttcggctgcgtttctgt

SEQ ID NO: 162 - DNA, 24 bp, Artificial Sequence
  Synthetic
ggcagagagagaaagggacatctt

SEQ ID NO: 163 - DNA, 23 bp, Artificial Sequence
  Synthetic
ctggagaagtggctgaatgatgc

SEQ ID NO: 164 - DNA, 23 bp, Artificial Sequence
  Synthetic
tttggtctcagtggaggtaggtg

SEQ ID NO: 165 - DNA, 20 bp, Artificial Sequence
  Synthetic
cagtcccatcactccaagga

SEQ ID NO: 166 - DNA, 21 bp, Artificial Sequence
  Synthetic
aggtccttggagtggaatgtg

SEQ ID NO: 167 - DNA, 22 bp, Artificial Sequence
  Synthetic
aaaggaggctggaaggttgtaa

SEQ ID NO: 168 - DNA, 23 bp, Artificial Sequence
  Synthetic
tgaaagaagccgacactaaacca

SEQ ID NO: 169 - DNA, 26 bp, Artificial Sequence
  Synthetic
catttctgcattctgcttaattccct

SEQ ID NO: 170 - DNA, 23 bp, Artificial Sequence
  Synthetic
ctttatctccacagacacgacat

SEQ ID NO: 171 - DNA, 25 bp, Artificial Sequence
  Synthetic
gtgcccgcttcttccatgccgtcct

SEQ ID NO: 172 - DNA, 21 bp, Artificial Sequence
  Synthetic
atgaggtttgccaagatgcca

SEQ ID NO: 173 - DNA, 22 bp, Artificial Sequence
  Synthetic
cattagcactatgtcatctgtg

SEQ ID NO: 174 - DNA, 21 bp, Artificial Sequence
  Synthetic
tcccgagattggatgatgtgc

SEQ ID NO: 175 - DNA, 22 bp, Artificial Sequence
  Synthetic
cctttcctccaaccgatgcttc

SEQ ID NO: 176 - DNA, 24 bp, Artificial Sequence
  Synthetic
ataggcaagtaggaggtgggcagc

SEQ ID NO: 177 - DNA, 23 bp, Artificial Sequence
  Synthetic
gacgggcaaggatgaggatgaga

SEQ ID NO: 178 - DNA, 29 bp, Artificial Sequence
  Synthetic
tttgtcaggaaagttgagcatttgttggg

SEQ ID NO: 179 - DNA, 23 bp, Artificial Sequence
  Synthetic
cgtgtagtgaatgggaaagtgga

SEQ ID NO: 180 - DNA, 25 bp, Artificial Sequence
  Synthetic
tttgcttggaataatggcatctcag

SEQ ID NO: 181 - DNA, 23 bp, Artificial Sequence
  Synthetic
ggcagaagcccgtgaagttccag

SEQ ID NO: 182 - DNA, 25 bp, Artificial Sequence
  Synthetic
tggtcatcaaagcaaagggaaaggt

SEQ ID NO: 183 - DNA, 23 bp, Artificial Sequence
  Synthetic
gacagagcagaccaatcacatta

SEQ ID NO: 184 - DNA, 27 bp, Artificial Sequence
  Synthetic
tactcataactggatttcctgactgac

SEQ ID NO: 185 - DNA, 24 bp, Artificial Sequence
  Synthetic
gagatctgtttgtttgataggaga

SEQ ID NO: 186 - DNA, 24 bp, Artificial Sequence
  Synthetic
gttcttttaacttagggagcagct

SEQ ID NO: 187 - DNA, 24 bp, Artificial Sequence
  Synthetic
tgtattactgctccgtggtctcag

SEQ ID NO: 188 - DNA, 21 bp, Artificial Sequence
  Synthetic
tctcctcccaccattactcgt

SEQ ID NO: 189 - DNA, 24 bp, Artificial Sequence
  Synthetic
gtccagatagacaagcagagatgc

SEQ ID NO: 190 - DNA, 20 bp, Artificial Sequence
  Synthetic
aacctccagtcgcagccttc

SEQ ID NO: 191 - DNA, 22 bp, Artificial Sequence
  Synthetic
acagaacaggcattcaggagtc

SEQ ID NO: 192 - DNA, 21 bp, Artificial Sequence
  Synthetic
gagcataggagaactggttgc

SEQ ID NO: 193 - DNA, 21 bp, Artificial Sequence
  Synthetic
gacggctgggctgctgctggg

SEQ ID NO: 194 - DNA, 20 bp, Artificial Sequence
  Synthetic
gactcagttcagcctcaggg

SEQ ID NO: 195 - DNA, 23 bp, Artificial Sequence
  Synthetic
ggcccctggatggatagctactc

SEQ ID NO: 196 - DNA, 23 bp, Artificial Sequence
  Synthetic
gcctcattcggacacactggctg

SEQ ID NO: 197 - DNA, 22 bp, Artificial Sequence
  Synthetic
ggccccattcgctgtgaccgct

SEQ ID NO: 198 - DNA, 22 bp, Artificial Sequence
  Synthetic
ggccacataactgcactgatca

SEQ ID NO: 199 - DNA, 22 bp, Artificial Sequence
  Synthetic
gaggtcttattgcgggtaaagg

SEQ ID NO: 200 - DNA, 21 bp, Artificial Sequence
  Synthetic
gtgctcaacttggatgggaca

SEQ ID NO: 201 - DNA, 22 bp, Artificial Sequence
  Synthetic
ccaaaatcaggggatcgcagcg

SEQ ID NO: 202 - DNA, 21 bp, Artificial Sequence
  Synthetic
ggatgttgcctaatgagccac

SEQ ID NO: 203 - DNA, 22 bp, Artificial Sequence
  Synthetic
gaagaggtggtggaagagtacg

SEQ ID NO: 204 - DNA, 22 bp, Artificial Sequence
  Synthetic
tcggtctcagcctctgcttcag

SEQ ID NO: 205 - DNA, 21 bp, Artificial Sequence
  Synthetic
ggtttacagtgatgcccagcc

SEQ ID NO: 206 - DNA, 22 bp, Artificial Sequence
  Synthetic
gtgtgcagatgggattaacgtc

SEQ ID NO: 207 - DNA, 23 bp, Artificial Sequence
  Synthetic
cccaatagaattacccgccaagc

SEQ ID NO: 208 - DNA, 21 bp, Artificial Sequence
  Synthetic
tgttttggcaggacagtgagc

SEQ ID NO: 209 - DNA, 20 bp, Artificial Sequence
  Synthetic
gatgtactgagaatgtgccc

SEQ ID NO: 210 - DNA, 22 bp, Artificial Sequence
  Synthetic
gagtttactggtgatcgctgcc

SEQ ID NO: 211 - DNA, 21 bp, Artificial Sequence
  Synthetic
tcaccagctggacattctcgg

SEQ ID NO: 212 - DNA, 23 bp, Artificial Sequence
  Synthetic
ggaggacttctacccggaacatc

SEQ ID NO: 213 - DNA, 24 bp, Artificial Sequence
  Synthetic
cagtgtactggatgctcttcaggg

SEQ ID NO: 214 - DNA, 23 bp, Artificial Sequence
  Synthetic
cagatagagagcaggcatagaca

SEQ ID NO: 215 - DNA, 20 bp, Artificial Sequence
  Synthetic
tgaggaggaaagggcgtttg

SEQ ID NO: 216 - DNA, 23 bp, Artificial Sequence
  Synthetic
gtgctgaagggacattgtgagaa

SEQ ID NO: 217 - DNA, 20 bp, Artificial Sequence
  Synthetic
gactcgcttattcacttctg

SEQ ID NO: 218 - DNA, 20 bp, Artificial Sequence
  Synthetic
gtggtggatgacggggtgac

SEQ ID NO: 219 - DNA, 20 bp, Artificial Sequence
  Synthetic
cggactcggacgcgtggtag

SEQ ID NO: 220 - DNA, 20 bp, Artificial Sequence
  Synthetic
cgccaccaccaggatgtagg

SEQ ID NO: 221 - DNA, 22 bp, Artificial Sequence
  Synthetic
tgtttgaaatgagcaggcactc

SEQ ID NO: 222 - DNA, 22 bp, Artificial Sequence
  Synthetic
gttccgacttggtttgtcttgt

SEQ ID NO: 223 - DNA, 20 bp, Artificial Sequence
  Synthetic
gctggcacctctcaaacctg

SEQ ID NO: 224 - DNA, 20 bp, Artificial Sequence
  Synthetic
aggcttttcatcactgtcac

SEQ ID NO: 225 - DNA, 20 bp, Artificial Sequence
  Synthetic
aggcttttcatcactgtcac

SEQ ID NO: 226 - DNA, 20 bp, Artificial Sequence
  Synthetic
tctggctttccttgctattg

SEQ ID NO: 227 - DNA, 26 bp, Artificial Sequence
  Synthetic
gcgtctcccactacatcatcaacagc

SEQ ID NO: 228 - DNA, 25 bp, Artificial Sequence
  Synthetic
ctaacacacaagccctccagttcgt

SEQ ID NO: 229 - DNA, 21 bp, Artificial Sequence
  Synthetic
gcctcagaccagaaagtgaag

SEQ ID NO: 230 - DNA, 22 bp, Artificial Sequence
  Synthetic
gaaatccatagaccttgtggcg

SEQ ID NO: 231 - DNA, 22 bp, Artificial Sequence
  Synthetic
gatgcggagtctacgatgggac

SEQ ID NO: 232 - DNA, 20 bp, Artificial Sequence
  Synthetic
actttccagtgagttccagc

SEQ ID NO: 233 - DNA, 22 bp, Artificial Sequence
  Synthetic
tgagttacctgaccttggacca

SEQ ID NO: 234 - DNA, 23 bp, Artificial Sequence
  Synthetic
ttcctggagttgggagtgaagtg

SEQ ID NO: 235 - DNA, 19 bp, Artificial Sequence
  Synthetic
ggcccgactctatcacaag

SEQ ID NO: 236 - DNA, 21 bp, Artificial Sequence
  Synthetic
tcctttcacccattcagtggc

SEQ ID NO: 237 - DNA, 22 bp, Artificial Sequence
  Synthetic
ctcagcagtcttagtgggtatc

SEQ ID NO: 238 - DNA, 22 bp, Artificial Sequence
  Synthetic
gagaatggagagttggcacctg

SEQ ID NO: 239 - DNA, 20 bp, Artificial Sequence
  Synthetic
tgttcgcgcctggtagagat

SEQ ID NO: 240 - DNA, 21 bp, Artificial Sequence
  Synthetic
tttggttgatgatggctggac

SEQ ID NO: 241 - DNA, 23 bp, Artificial Sequence
  Synthetic
ctatggaatcgcagacggttgat

SEQ ID NO: 242 - DNA, 23 bp, Artificial Sequence
  Synthetic
gcaagaagaaagagaagcagggc

SEQ ID NO: 243 - DNA, 24 bp, Artificial Sequence
  Synthetic
cacgctcgtttctcttgttcacat

SEQ ID NO: 244 - DNA, 23 bp, Artificial Sequence
  Synthetic
gctcgtcgtcctcatcaaactca

SEQ ID NO: 245 - DNA, 20 bp, Artificial Sequence
  Synthetic
gccaacagcacctccacaga

SEQ ID NO: 246 - DNA, 20 bp, Artificial Sequence
  Synthetic
agcagggagctgggaatggt

SEQ ID NO: 247 - DNA, 21 bp, Artificial Sequence
  Synthetic
gcgatgaaaccaggaactcac

SEQ ID NO: 248 - DNA, 21 bp, Artificial Sequence
  Synthetic
ggaaggctggtgtctctgtta

SEQ ID NO: 249 - DNA, 23 bp, Artificial Sequence
  Synthetic
cctaagcagctggaaggaaccat

SEQ ID NO: 250 - DNA, 23 bp, Artificial Sequence
  Synthetic
gatttcctacagcctggtcctct

SEQ ID NO: 251 - DNA, 24 bp, Artificial Sequence
  Synthetic
agaggaagaccgccaaagaacatc

SEQ ID NO: 252 - DNA, 20 bp, Artificial Sequence
  Synthetic
gatagagcaggcactcggca

SEQ ID NO: 253 - DNA, 20 bp, Artificial Sequence
  Synthetic
agtccctgtgagacggtttc

SEQ ID NO: 254 - DNA, 20 bp, Artificial Sequence
  Synthetic
actgtctttgttgctccctc

SEQ ID NO: 255 - DNA, 19 bp, Artificial Sequence
  Synthetic
gcatgggataacgcggcca

SEQ ID NO: 256 - DNA, 18 bp, Artificial Sequence
  Synthetic
cctcaggaagcgcatgcg

SEQ ID NO: 257 - DNA, 20 bp, Artificial Sequence
  Synthetic
agaaggagtggacggtgagt

SEQ ID NO: 258 - DNA, 20 bp, Artificial Sequence
  Synthetic
gcttgttagagtgctgtgca

SEQ ID NO: 259 - DNA, 20 bp, Artificial Sequence
  Synthetic
ggaaaccagtgtagctgcag

SEQ ID NO: 260 - DNA, 24 bp, Artificial Sequence
  Synthetic
gttgtttctacacctgtgctatgg

SEQ ID NO: 261 - DNA, 24 bp, Artificial Sequence
  Synthetic
gtattatgggagcatctgaggtca

SEQ ID NO: 262 - DNA, 20 bp, Artificial Sequence
  Synthetic
gctgctctgaaggaacaaac

SEQ ID NO: 263 - DNA, 20 bp, Artificial Sequence
  Synthetic
gataaatgaggggcgaaatg

SEQ ID NO: 264 - DNA, 23 bp, Artificial Sequence
  Synthetic
ccacagcggcaaggtctccaagt

SEQ ID NO: 265 - DNA, 26 bp, Artificial Sequence
  Synthetic
gcctttgctaaactgtccatttccga

SEQ ID NO: 266 - DNA, 22 bp, Artificial Sequence
  Synthetic
gccgcttcctgctcaactccag

SEQ ID NO: 267 - DNA, 20 bp, Artificial Sequence
  Synthetic
gcctcaatccttcttgctcc

SEQ ID NO: 268 - DNA, 22 bp, Artificial Sequence
  Synthetic
ccctgttggagagactatggcg

SEQ ID NO: 269 - DNA, 22 bp, Artificial Sequence
  Synthetic
atccgctgtccgaactcaatgg

SEQ ID NO: 270 - DNA, 21 bp, Artificial Sequence
  Synthetic
aaatcgggctgaagcgactga

SEQ ID NO: 271 - DNA, 23 bp, Artificial Sequence
  Synthetic
tttggctcaactactctcccatc

SEQ ID NO: 272 - DNA, 20 bp, Artificial Sequence
  Synthetic
gagttccaggcttctgccaa

SEQ ID NO: 273 - DNA, 26 bp, Artificial Sequence
  Synthetic
ttcaccaaagtattgttaattagcag

SEQ ID NO: 274 - DNA, 24 bp, Artificial Sequence
  Synthetic
ttcatcgggagactaaatccagcg

SEQ ID NO: 275 - DNA, 24 bp, Artificial Sequence
  Synthetic
ccataagaggcaaactcaaccacc

SEQ ID NO: 276 - DNA, 22 bp, Artificial Sequence
  Synthetic
gggtgactcttctcaaatgcct

SEQ ID NO: 277 - DNA, 21 bp, Artificial Sequence
  Synthetic
gcatttacagcactcacggac

SEQ ID NO: 278 - DNA, 22 bp, Artificial Sequence
  Synthetic
gctccccagaggacacagattc

SEQ ID NO: 279 - DNA, 21 bp, Artificial Sequence
  Synthetic
gctccccagaggacacagcct

SEQ ID NO: 280 - DNA, 22 bp, Artificial Sequence
  Synthetic
gctcctcctcggtcatctctac

SEQ ID NO: 281 - DNA, 20 bp, Artificial Sequence
  Synthetic
ataccatctccgtggatcgg

SEQ ID NO: 282 - DNA, 23 bp, Artificial Sequence
  Synthetic
tttgcctttgccctcctctgact

SEQ ID NO: 283 - DNA, 24 bp, Artificial Sequence
  Synthetic
ctgcccaacgcaccgaatagttac

SEQ ID NO: 284 - DNA, 25 bp, Artificial Sequence
  Synthetic
gagcctctctggttctttcaatcgg

SEQ ID NO: 285 - DNA, 22 bp, Artificial Sequence
  Synthetic
gttctcgccaccaaagtccttg

SEQ ID NO: 286 - DNA, 20 bp, Artificial Sequence
  Synthetic
aggaggtcccctgcttgggc

SEQ ID NO: 287 - DNA, 21 bp, Artificial Sequence
  Synthetic
ggaggtcccctgctacggtac

SEQ ID NO: 288 - DNA, 21 bp, Artificial Sequence
  Synthetic
cgaagaaagtcctggttgcac

SEQ ID NO: 289 - DNA, 22 bp, Artificial Sequence
  Synthetic
ggatacgctggggagtttattc

SEQ ID NO: 290 - DNA, 22 bp, Artificial Sequence
  Synthetic
ctgctctggtggagtatggcac

SEQ ID NO: 291 - DNA, 22 bp, Artificial Sequence
  Synthetic
tgccgtccagacactcatagcc

SEQ ID NO: 292 - DNA, 20 bp, Artificial Sequence
  Synthetic
tctgcaaagaccatgactcc

SEQ ID NO: 293 - DNA, 21 bp, Artificial Sequence
  Synthetic
agcatggatgggtccaagtcc

SEQ ID NO: 294 - DNA, 20 bp, Artificial Sequence
  Synthetic
cccacttcgttcaagacagg

SEQ ID NO: 295 - DNA, 20 bp, Artificial Sequence
  Synthetic
atccaatcccacagcgagag

SEQ ID NO: 296 - DNA, 20 bp, Artificial Sequence
  Synthetic
cctacacctgaaaaacaaga

SEQ ID NO: 297 - DNA, 20 bp, Artificial Sequence
  Synthetic
gctgtgtgaccccaaactgc

SEQ ID NO: 298 - DNA, 23 bp, Artificial Sequence
  Synthetic
actacaactcactgacccgctca

SEQ ID NO: 299 - DNA, 21 bp, Artificial Sequence
  Synthetic
tcctccatcctgggactctat

SEQ ID NO: 300 - DNA, 22 bp, Artificial Sequence
  Synthetic
ctgaaagggaagagagttggct

SEQ ID NO: 301 - DNA, 20 bp, Artificial Sequence
  Synthetic
tatcattctggtcggcttca

SEQ ID NO: 302 - DNA, 19 bp, Artificial Sequence
  Synthetic
gggctgcttgctaactcca

SEQ ID NO: 303 - DNA, 21 bp, Artificial Sequence
  Synthetic
atgtggctggctttgacactc

SEQ ID NO: 304 - DNA, 22 bp, Artificial Sequence
  Synthetic
gccatcgtctgctacattaccc

SEQ ID NO: 305 - DNA, 20 bp, Artificial Sequence
  Synthetic
agcagcctcctctcagatcc

SEQ ID NO: 306 - DNA, 21 bp, Artificial Sequence
  Synthetic
cctacaagcggcacaaggatg

SEQ ID NO: 307 - DNA, 20 bp, Artificial Sequence
  Synthetic
ccgtgatatcagtgggatgg

SEQ ID NO: 308 - DNA, 20 bp, Artificial Sequence
  Synthetic
tactgggagggcattgacca

SEQ ID NO: 309 - DNA, 21 bp, Artificial Sequence
  Synthetic
tccgaatgtcacgaacctcct

SEQ ID NO: 310 - DNA, 22 bp, Artificial Sequence
  Synthetic
ttcaaaccacacaggctatgcc

SEQ ID NO: 311 - DNA, 20 bp, Artificial Sequence
  Synthetic
atgtccatcacccgcaaggc

SEQ ID NO: 312 - DNA, 24 bp, Artificial Sequence
  Synthetic
caaatggagcaggaacttcaggac

SEQ ID NO: 313 - DNA, 20 bp, Artificial Sequence
  Synthetic
ttcagagcagcggagtcacg

SEQ ID NO: 314 - DNA, 22 bp, Artificial Sequence
  Synthetic
ccagaggagaaggaaggagcag

SEQ ID NO: 315 - DNA, 25 bp, Artificial Sequence
  Synthetic
ggaacactttcatcatctcccacag

SEQ ID NO: 316 - DNA, 17 bp, Artificial Sequence
  Synthetic
ggcggcgtaggacggag

SEQ ID NO: 317 - DNA, 20 bp, Artificial Sequence
  Synthetic
cgaggcaatcagctccaatc

SEQ ID NO: 318 - DNA, 23 bp, Artificial Sequence
  Synthetic
cttgattgaggtggagcgaaagt

SEQ ID NO: 319 - DNA, 22 bp, Artificial Sequence
  Synthetic
gcaccgcacaacgggcgtaata

SEQ ID NO: 320 - DNA, 23 bp, Artificial Sequence
  Synthetic
gcctctacctcacccacagcgta

SEQ ID NO: 321 - DNA, 21 bp, Artificial Sequence
  Synthetic
cttggctggtgctgtctcctg

SEQ ID NO: 322 - DNA, 22 bp, Artificial Sequence
  Synthetic
gtgaggactagaggaagaatgc

SEQ ID NO: 323 - DNA, 20 bp, Artificial Sequence
  Synthetic
gccctactctattgcctaag

SEQ ID NO: 324 - DNA, 24 bp, Artificial Sequence
  Synthetic
gtgttctccgagttcctgtctctc

SEQ ID NO: 325 - DNA, 22 bp, Artificial Sequence
  Synthetic
gggaagggagaagttgagtcgg

SEQ ID NO: 326 - DNA, 22 bp, Artificial Sequence
  Synthetic
cattgtaagggtagccactggg

SEQ ID NO: 327 - DNA, 27 bp, Artificial Sequence
  Synthetic
gtacacccacctccacccttatatcct

SEQ ID NO: 328 - DNA, 23 bp, Artificial Sequence
  Synthetic
gccaccagtgaccatcccaacaa

SEQ ID NO: 329 - DNA, 21 bp, Artificial Sequence
  Synthetic
accgccagcctcttattccat

SEQ ID NO: 330 - DNA, 20 bp, Artificial Sequence
  Synthetic
ttgtgggatggtggagtcct

SEQ ID NO: 331 - DNA, 24 bp, Artificial Sequence
  Synthetic
ttctcgagcagcggcagttctcac

SEQ ID NO: 332 - DNA, 22 bp, Artificial Sequence
  Synthetic
cacacagtctgtaagctttccc

SEQ ID NO: 333 - DNA, 22 bp, Artificial Sequence
  Synthetic
aatcaggacccacctctctgcc

SEQ ID NO: 334 - DNA, 21 bp, Artificial Sequence
  Synthetic
ggctggttctttggcttcctg

SEQ ID NO: 335 - DNA, 19 bp, Artificial Sequence
  Synthetic
tccatcaacacgctctcgg

SEQ ID NO: 336 - DNA, 20 bp, Artificial Sequence
  Synthetic
actgtcgtgcttgctcagga

SEQ ID NO: 337 - DNA, 21 bp, Artificial Sequence
  Synthetic
catcggatttgagacctgcag

SEQ ID NO: 338 - DNA, 22 bp, Artificial Sequence
  Synthetic
cttcgactgttgactgcaatgc

SEQ ID NO: 339 - DNA, 25 bp, Artificial Sequence
  Synthetic
ccatgaagctgacgcggaagatggt

SEQ ID NO: 340 - DNA, 24 bp, Artificial Sequence
  Synthetic
ctcctcctccgtcacagcctggtt

SEQ ID NO: 341 - DNA, 25 bp, Artificial Sequence
  Synthetic
gggacaggactggtgtagacaggca

SEQ ID NO: 342 - DNA, 19 bp, Artificial Sequence
  Synthetic
gagcgtgaggcagatcggc

SEQ ID NO: 343 - DNA, 22 bp, Artificial Sequence
  Synthetic
ccgaaaccacaaaccttgccat

SEQ ID NO: 344 - DNA, 21 bp, Artificial Sequence
  Synthetic
gcaggagtgaaaggactgacc

SEQ ID NO: 345 - DNA, 25 bp, Artificial Sequence
  Synthetic
gcccatcttctactccttggctaac

SEQ ID NO: 346 - DNA, 24 bp, Artificial Sequence
  Synthetic
ccgtgttgtcctacctgaagttct

SEQ ID NO: 347 - DNA, 23 bp, Artificial Sequence
  Synthetic
gtaggtgtcctggtgggaatgaa

SEQ ID NO: 348 - DNA, 23 bp, Artificial Sequence
  Synthetic
ctttgacctctgcttcctggtgc

SEQ ID NO: 349 - DNA, 23 bp, Artificial Sequence
  Synthetic
ttgcggaccacagcattctcatc

SEQ ID NO: 350 - DNA, 24 bp, Artificial Sequence
  Synthetic
gaaagaggaaagacacagagagac

SEQ ID NO: 351 - DNA, 20 bp, Artificial Sequence
  Synthetic
tggaggattctggctcagga

SEQ ID NO: 352 - DNA, 24 bp, Artificial Sequence
  Synthetic
tccttctatgacctcagtgccatc

SEQ ID NO: 353 - DNA, 23 bp, Artificial Sequence
  Synthetic
atgttgatggttgggaaggtgcg

SEQ ID NO: 354 - DNA, 24 bp, Artificial Sequence
  Synthetic
gacgctgttcttccatctttactc

SEQ ID NO: 355 - DNA, 24 bp, Artificial Sequence
  Synthetic
ttacccaagaatcaggaatggaac

SEQ ID NO: 356 - DNA, 24 bp, Artificial Sequence
  Synthetic
gttacaatgctgctaagaggagga

SEQ ID NO: 357 - DNA, 23 bp, Artificial Sequence
  Synthetic
tccaaacacaagactcatctacc

SEQ ID NO: 358 - DNA, 23 bp, Artificial Sequence
  Synthetic
ggtagtgtttggtgtccctgtct

SEQ ID NO: 359 - DNA, 23 bp, Artificial Sequence
  Synthetic
ggtagtgtttggtgtccctgtct

SEQ ID NO: 360 - DNA, 24 bp, Artificial Sequence
  Synthetic
gaactggatgaaagcaaacaacac

SEQ ID NO: 361 - DNA, 24 bp, Artificial Sequence
  Synthetic
ccttagcctttgcttcatcgtctc

SEQ ID NO: 362 - DNA, 25 bp, Artificial Sequence
  Synthetic
caaggaatgcttctccctgtatgac

SEQ ID NO: 363 - DNA, 24 bp, Artificial Sequence
  Synthetic
gtttgccatctctcccaagtgaaa

SEQ ID NO: 364 - DNA, 23 bp, Artificial Sequence
  Synthetic
tggaagaccagagagagggtttg

SEQ ID NO: 365 - DNA, 23 bp, Artificial Sequence
  Synthetic
cacttctgtctggcgattctgtg

SEQ ID NO: 366 - DNA, 24 bp, Artificial Sequence
  Synthetic
agaggcagagagaggagacacgca

SEQ ID NO: 367 - DNA, 27 bp, Artificial Sequence
  Synthetic
cctcatcttcctctcttggataaccca

SEQ ID NO: 368 - DNA, 25 bp, Artificial Sequence
  Synthetic
gaaaagtggcccgagaattccggca

SEQ ID NO: 369 - DNA, 21 bp, Artificial Sequence
  Synthetic
ttctcctttgccgctccatct

SEQ ID NO: 370 - DNA, 21 bp, Artificial Sequence
  Synthetic
gacgaagactacagcaccatc

SEQ ID NO: 371 - DNA, 22 bp, Artificial Sequence
  Synthetic
aaggagtcgtgttcaatgtcac

SEQ ID NO: 372 - DNA, 21 bp, Artificial Sequence
  Synthetic
ctgagagaaaggacagttgcc

SEQ ID NO: 373 - DNA, 21 bp, Artificial Sequence
  Synthetic
tgaactcatcacgggcatagg

SEQ ID NO: 374 - DNA, 26 bp, Artificial Sequence
  Synthetic
atcatcaggatacagagacatcggta

SEQ ID NO: 375 - DNA, 26 bp, Artificial Sequence
  Synthetic
gcaagtgatttcagaatgttgtaggc

SEQ ID NO: 376 - DNA, 23 bp, Artificial Sequence
  Synthetic
ccaaagcggcactcaactgaagg

SEQ ID NO: 377 - DNA, 26 bp, Artificial Sequence
  Synthetic
cagcctgggataaggtttcagatgtc

SEQ ID NO: 378 - DNA, 24 bp, Artificial Sequence
  Synthetic
gagagccgccaggaagagatgaat

SEQ ID NO: 379 - DNA, 24 bp, Artificial Sequence
  Synthetic
tcctggcttcttctccttcagtcg

SEQ ID NO: 380 - DNA, 23 bp, Artificial Sequence
  Synthetic
gctttcaaggtgtggatttggct

SEQ ID NO: 381 - DNA, 24 bp, Artificial Sequence
  Synthetic
ggcactgatctggactgtcaggtt

SEQ ID NO: 382 - DNA, 23 bp, Artificial Sequence
  Synthetic
aaactcttcgctcacaccacccg

SEQ ID NO: 383 - DNA, 24 bp, Artificial Sequence
  Synthetic
gcaaacttctccgcatccatcgtg

SEQ ID NO: 384 - DNA, 23 bp, Artificial Sequence
  Synthetic
cagcaccgaggaagcatttatga

SEQ ID NO: 385 - DNA, 26 bp, Artificial Sequence
  Synthetic
aatctcttcttccctttgtcgtttcc

SEQ ID NO: 386 - DNA, 24 bp, Artificial Sequence
  Synthetic
cttggcgggtgaaggtgtgtgtca

SEQ ID NO: 387 - DNA, 29 bp, Artificial Sequence
  Synthetic
ggttacactttacagacatcacaaatccc

SEQ ID NO: 388 - DNA, 26 bp, Artificial Sequence
  Synthetic
gtgcggatgtcgggctgggcggacga

SEQ ID NO: 389 - DNA, 26 bp, Artificial Sequence
  Synthetic
cttgacccagaccgagaccgtgagta

SEQ ID NO: 390 - DNA, 23 bp, Artificial Sequence
  Synthetic
tgtccctaatctgctccatctct

SEQ ID NO: 391 - DNA, 23 bp, Artificial Sequence
  Synthetic
gaccgaccaaatcctgatagtgg

SEQ ID NO: 392 - DNA, 24 bp, Artificial Sequence
  Synthetic
ccctgcacaagccctcctgcccat

SEQ ID NO: 393 - DNA, 24 bp, Artificial Sequence
  Synthetic
accatgaacggggaggccatctgc

SEQ ID NO: 394 - DNA, 20 bp, Artificial Sequence
  Synthetic
gtgagagggacatcatgcgc

SEQ ID NO: 395 - DNA, 20 bp, Artificial Sequence
  Synthetic
aatccaggcacggaactcaa

SEQ ID NO: 396 - DNA, 21 bp, Artificial Sequence
  Synthetic
gcgataatcacaaccaccacg

SEQ ID NO: 397 - DNA, 21 bp, Artificial Sequence
  Synthetic
accaggatgaggagcattgcc

SEQ ID NO: 398 - DNA, 20 bp, Artificial Sequence
  Synthetic
atcagtgggtgggaggtgag

SEQ ID NO: 399 - DNA, 21 bp, Artificial Sequence
  Synthetic
ggggcgccataaggaggaagc

SEQ ID NO: 400 - DNA, 24 bp, Artificial Sequence
  Synthetic
ctggaactgtccgttcagtccatc

SEQ ID NO: 401 - DNA, 22 bp, Artificial Sequence
  Synthetic
gatggacgggtccggggagcag

SEQ ID NO: 402 - DNA, 27 bp, Artificial Sequence
  Synthetic
ctcagcccatcttcttccagatggtga

SEQ ID NO: 403 - DNA, 25 bp, Artificial Sequence
  Synthetic
ggcagctgatcatagatctggagac

SEQ ID NO: 404 - DNA, 22 bp, Artificial Sequence
  Synthetic
caggggaagtggaggccacctc

SEQ ID NO: 405 - DNA, 20 bp, Artificial Sequence
  Synthetic
gtgggacggcagctcgccat

SEQ ID NO: 406 - DNA, 20 bp, Artificial Sequence
  Synthetic
ggccatgctggtagacgtgt

SEQ ID NO: 407 - DNA, 24 bp, Artificial Sequence
  Synthetic
gcaaccgggagctggtggttgact

SEQ ID NO: 408 - DNA, 23 bp, Artificial Sequence
  Synthetic
ctggtcatttccgactgaagagt

SEQ ID NO: 409 - DNA, 21 bp, Artificial Sequence
  Synthetic
gtggaactcctcaacttgctg

SEQ ID NO: 410 - DNA, 23 bp, Artificial Sequence
  Synthetic
ggtcaaccccacgatcagtctca

SEQ ID NO: 411 - DNA, 22 bp, Artificial Sequence
  Synthetic
gaggcgacagtgaaaccctttg

SEQ ID NO: 412 - DNA, 22 bp, Artificial Sequence
  Synthetic
gtgctccagtctctctcggatg

SEQ ID NO: 413 - DNA, 21 bp, Artificial Sequence
  Synthetic
ttggtcctgattccctcacgg

SEQ ID NO: 414 - DNA, 22 bp, Artificial Sequence
  Synthetic
cccatatgctaccaagcgtgag

SEQ ID NO: 415 - DNA, 23 bp, Artificial Sequence
  Synthetic
ctggaaggtaggagagctgtctg

SEQ ID NO: 416 - DNA, 20 bp, Artificial Sequence
  Synthetic
ccatcgtttcttgggtcgtc

SEQ ID NO: 417 - DNA, 20 bp, Artificial Sequence
  Synthetic
ggtagttggtggagagcagg

SEQ ID NO: 418 - DNA, 22 bp, Artificial Sequence
  Synthetic
caacagttcgtgcttcagtagg

SEQ ID NO: 419 - DNA, 22 bp, Artificial Sequence
  Synthetic
ggtggcagagacaagtaatagg

SEQ ID NO: 420 - DNA, 22 bp, Artificial Sequence
  Synthetic
tccgtgctgtttgtgtgtctgg

SEQ ID NO: 421 - DNA, 23 bp, Artificial Sequence
  Synthetic
gctttatgggctgtgtgaatgcc

SEQ ID NO: 422 - DNA, 24 bp, Artificial Sequence
  Synthetic
gtgtggttggagttgatgtgttgg

SEQ ID NO: 423 - DNA, 23 bp, Artificial Sequence
  Synthetic
ctgctgccattggagtccttatg

SEQ ID NO: 424 - DNA, 22 bp, Artificial Sequence
  Synthetic
gaagccagtccagagcctaagg

SEQ ID NO: 425 - DNA, 20 bp, Artificial Sequence
  Synthetic
agccaatgacaggaagtgtg

SEQ ID NO: 426 - DNA, 20 bp, Artificial Sequence
  Synthetic
tgaggagcagacccaggcat

SEQ ID NO: 427 - DNA, 22 bp, Artificial Sequence
  Synthetic
cttctgggagcacttgggacag

SEQ ID NO: 428 - DNA, 20 bp, Artificial Sequence
  Synthetic
gactcctggcatcagaacca

SEQ ID NO: 429 - DNA, 22 bp, Artificial Sequence
  Synthetic
cccttcaccatcttcctcactc

SEQ ID NO: 430 - DNA, 20 bp, Artificial Sequence
  Synthetic
ggcgaggacacacggcttag

SEQ ID NO: 431 - DNA, 20 bp, Artificial Sequence
  Synthetic
aacacaaacggagcaatgac

SEQ ID NO: 432 - DNA, 20 bp, Artificial Sequence
  Synthetic
ccagaggagtgcggggaacc

SEQ ID NO: 433 - DNA, 20 bp, Artificial Sequence
  Synthetic
acgcctttccccactgttac

SEQ ID NO: 434 - DNA, 20 bp, Artificial Sequence
  Synthetic
tcaggctggtggttgctgga

SEQ ID NO: 435 - DNA, 20 bp, Artificial Sequence
  Synthetic
cgaacaccctggaccctctg

SEQ ID NO: 436 - DNA, 20 bp, Artificial Sequence
  Synthetic
gatgtgttggttggagaaag

SEQ ID NO: 437 - DNA, 20 bp, Artificial Sequence
  Synthetic
cttggatttgccgagactgg

SEQ ID NO: 438 - DNA, 20 bp, Artificial Sequence
  Synthetic
gcctggagcccgccgagaac

SEQ ID NO: 439 - DNA, 20 bp, Artificial Sequence
  Synthetic
ggcggcttctacatcacctc

SEQ ID NO: 440 - DNA, 21 bp, Artificial Sequence
  Synthetic
gctggggatgtagcctgtctg

SEQ ID NO: 441 - DNA, 20 bp, Artificial Sequence
  Synthetic
accacagcatacaactgcac

SEQ ID NO: 442 - DNA, 20 bp, Artificial Sequence
  Synthetic
ggtccacgcccgcttctgta

SEQ ID NO: 443 - DNA, 20 bp, Artificial Sequence
  Synthetic
tgacctgggcacctctcttc

SEQ ID NO: 444 - DNA, 20 bp, Artificial Sequence
  Synthetic
ttgggatttgggtcttttgt

SEQ ID NO: 445 - DNA, 20 bp, Artificial Sequence
  Synthetic
gcaaggcaagggatggatag

SEQ ID NO: 446 - DNA, 20 bp, Artificial Sequence
  Synthetic
gaacggaggaggagaggacc

SEQ ID NO: 447 - DNA, 21 bp, Artificial Sequence
  Synthetic
tagtggtgacggtggtgacag

SEQ ID NO: 448 - DNA, 22 bp, Artificial Sequence
  Synthetic
atgcgtatcccactgcctatgg

SEQ ID NO: 449 - DNA, 24 bp, Artificial Sequence
  Synthetic
aagatgctggtgtatgtgacgagg

SEQ ID NO: 450 - DNA, 23 bp, Artificial Sequence
  Synthetic
caatggcggctctgaagagttgg

SEQ ID NO: 451 - DNA, 20 bp, Artificial Sequence
  Synthetic
cctggcggttatagaggcct

SEQ ID NO: 452 - DNA, 25 bp, Artificial Sequence
  Synthetic
ggcagggctcaaatttctgcctaca

SEQ ID NO: 453 - DNA, 25 bp, Artificial Sequence
  Synthetic
gattgttgatgatcagacagtatcc

SEQ ID NO: 454 - DNA, 23 bp, Artificial Sequence
  Synthetic
gtgctattgtgaggcggttgtag

SEQ ID NO: 455 - DNA, 21 bp, Artificial Sequence
  Synthetic
gactggatgaaccaggagcca

SEQ ID NO: 456 - DNA, 23 bp, Artificial Sequence
  Synthetic
ggctcctggcaacaggaccactg

SEQ ID NO: 457 - DNA, 23 bp, Artificial Sequence
  Synthetic
ggctcctggcaacaggaccactg

SEQ ID NO: 458 - DNA, 21 bp, Artificial Sequence
  Synthetic
ttctccgtggtagacaactcc

SEQ ID NO: 459 - DNA, 24 bp, Artificial Sequence
  Synthetic
gcgtgggggcagtcactatgctca

SEQ ID NO: 460 - DNA, 24 bp, Artificial Sequence
  Synthetic
ggggaccttgctgtagtcttcgga

SEQ ID NO: 461 - DNA, 24 bp, Artificial Sequence
  Synthetic
cccctactaccccatgcgtgcctc

SEQ ID NO: 462 - DNA, 20 bp, Artificial Sequence
  Synthetic
ggtgtcgttcaggacacagc

SEQ ID NO: 463 - DNA, 22 bp, Artificial Sequence
  Synthetic
gggcaattcgacgacgcggact

SEQ ID NO: 464 - DNA, 22 bp, Artificial Sequence
  Synthetic
cattcttgttctgggatccaac

SEQ ID NO: 465 - DNA, 22 bp, Artificial Sequence
  Synthetic
ctggagctggccgtcaacatgt

SEQ ID NO: 466 - DNA, 23 bp, Artificial Sequence
  Synthetic
ccaactcagtttccagatcctgg

SEQ ID NO: 467 - DNA, 22 bp, Artificial Sequence
  Synthetic
gggtctggccaggctatgacta

SEQ ID NO: 468 - DNA, 24 bp, Artificial Sequence
  Synthetic
gaggtcgccgttctccatgtagtc

SEQ ID NO: 469 - DNA, 24 bp, Artificial Sequence
  Synthetic
ccccaagacccttgtgctcgttgt

SEQ ID NO: 470 - DNA, 22 bp, Artificial Sequence
  Synthetic
gcaaagtcatcgaagcactgtc

SEQ ID NO: 471 - DNA, 22 bp, Artificial Sequence
  Synthetic
cccgaagatgataccattcctg

SEQ ID NO: 472 - DNA, 20 bp, Artificial Sequence
  Synthetic
gcagtgtcacactggctgcc

SEQ ID NO: 473 - DNA, 21 bp, Artificial Sequence
  Synthetic
ctactcagtgaagaagtgcat

SEQ ID NO: 474 - DNA, 20 bp, Artificial Sequence
  Synthetic
cgggaatcatcttccaccat

SEQ ID NO: 475 - DNA, 24 bp, Artificial Sequence
  Synthetic
cccagggggtgatcaagtacatgg

SEQ ID NO: 476 - DNA, 23 bp, Artificial Sequence
  Synthetic
gagtgtctgcattggttctacat

SEQ ID NO: 477 - DNA, 22 bp, Artificial Sequence
  Synthetic
ggaagatcgtcgccacctggat

SEQ ID NO: 478 - DNA, 25 bp, Artificial Sequence
  Synthetic
ggcatttccgtggcactaggtgtct

SEQ ID NO: 479 - DNA, 23 bp, Artificial Sequence
  Synthetic
cctctttgtgaagagatggcacc

SEQ ID NO: 480 - DNA, 22 bp, Artificial Sequence
  Synthetic
gccacttccgtgagatctcagt

SEQ ID NO: 481 - DNA, 21 bp, Artificial Sequence
  Synthetic
gccgctctacagcaaggtgac

SEQ ID NO: 482 - DNA, 23 bp, Artificial Sequence
  Synthetic
cctggctgtccagctagcagaga

SEQ ID NO: 483 - DNA, 23 bp, Artificial Sequence
  Synthetic
ccgccgaggagctcaagaagttc

SEQ ID NO: 484 - DNA, 22 bp, Artificial Sequence
  Synthetic
ggagcaagtccttgcaggtcca

SEQ ID NO: 485 - DNA, 25 bp, Artificial Sequence
  Synthetic
gggtctcctgttccaactccaccta

SEQ ID NO: 486 - DNA, 22 bp, Artificial Sequence
  Synthetic
ccaatggcaagttcaagtccac

SEQ ID NO: 487 - DNA, 24 bp, Artificial Sequence
  Synthetic
gctcggcttgtccagaagtgctta

SEQ ID NO: 488 - DNA, 21 bp, Artificial Sequence
  Synthetic
cggggttgtcatcttcctcct

SEQ ID NO: 489 - DNA, 25 bp, Artificial Sequence
  Synthetic
ccatgggtaacaacttctccagtat

SEQ ID NO: 490 - DNA, 25 bp, Artificial Sequence
  Synthetic
gggctaggagctgcggtaggtcttg

SEQ ID NO: 491 - DNA, 225 bp, Homo sapiens
tttggtgttaatgagtaccgccattggattaaagagtgtcttccttctcaggtggaagaattgcagcctttcatatcttcattaaacaaaccttatcatcttccccgtattctcattttacatattattatcatccaagagtaaactcaagtaagccaaaaagttaattttcgaagacttcaaacacctagagctattaaggagctagacaaaatagtggcatat

SEQ ID NO: 492 - DNA, 225 bp, Homo sapiens
tttggtgttaatgagtaccgccattggattaaagagaggtggaagaattgcagcctttcatatcttcattaaacaaaccttatcatcttccccgtattctcattttacatattattatcatccaagagtaaactcaagtaagccaaaaagttaattttcgaagacttcaaacacctagagctattaaggagctagacaaaatagtggcatatgaactaaaaactg

SEQ ID NO: 493 - DNA, 79 bp, Homo sapiens
ttaataaaactggcttcatctggcaagcagtctacagagacagcagctaatgtgaaagagctcgtgcagaatttactgg

SEQ ID NO: 494 - DNA, 75 bp, Homo sapiens
gacgatgatgacattaatgatgttgcatcgatggctggagtaaacttgtcagaagaaagtgcaagaatattagcc

SEQ ID NO: 495 - DNA, 93 bp, Homo sapiens
ctggatggaaaaatagaagcagaagatttcacaagcaggttataccgagaacttaattcttcacctcaaccttaccttgtgcctttcctgaag

SEQ ID NO: 496 - DNA, 75 bp, Homo sapiens
gtcatccagcagcctccgaagccaggagccctgatccggcccccgcaggtgacgttgacgcagacacccatggtc

SEQ ID NO: 497 - DNA, 963 bp, Homo sapiens
gagtgtgagctcgtgagtgggcgccgccgccaccgcccccgccgccgtcgtctcggtagcagccttcgccacgccggggtcttcaggtgagcaggccttgctctggtccaaggactccccattcccgacgccgactgcttactcaccagtcttggagcccgcaccgcgagggcccgcccccttggctgaccacgtgacccaactccactggggccatgtcagagcgagaagagcggcggtttgtggagatccctcgggagtctgtccggctcatggcggagagcacgggcctggagctgagcgatgaggtggcggcgctgctcgcagaggacgtgtgctatcgtctgagagaggccacgcagaatagctctcagttcatgaagcacaccaaacgccggaagctgacggttgaggacttcaacagggccctcagatggagcagcgtggaggctgtgtgtggttacggatcacaggaggcactgcccatgcgccccgccagggagggtgaactctactttcctgaggatcgagaggtgaacctggtggagctggccctggctaccaacatccccaaaggctgtgctgagacagctgtcagagttcatgtctcctacctggatggcaaagggaacctggcacctcaaggatcggtgcccagtgctgtgtcttcactgacagatgaccttctcaagtactatcaccaggtgactcgtgctgtgctaggggatgatccgcaactgatgaaggttgcactccaggacttgcagacgaactccaagattggggcactcctgccttactttgtttatgtggtcagtggggtgaaatctgtaagccatgacctggagcaactgcaccggctgctgcaggtggcacggagcctatttcgtaatccgcacctgtgcttggggccctatgtccgctgtctggtgggcagtgtcctctactgtgtcctggagccac

SEQ ID NO: 498 - DNA, 1380 bp, Homo sapiens
gagtgtgagctcgtgagtgggcgccgccgccaccgcccccgccgccgtcgtctcggtagcagccttcgccacgccggggtcttcagctccactggggccatgtcagagcgagaagagcggcggtttgtggagatccctcgggagtctgtccggctcatggcggagagcacgggcctggagctgagcgatgaggtggcggcgctgctcgcagaggacgtgtgctatcgtctgagagaggccacgcagaatagctctcagttcatgaagcacaccaaacgccggaagctgacggttgaggacttcaacagggccctcagatggagcagcgtggaggctgtgtgtggttacggatcacaggaggcactgcccatgcgccccgccagggagggtgaactctactttcctgaggatcgagaggtgaacctggtggagctggccctggctaccaacatccccaaaggctgtgctgagacagctgtcagagttcatgtctcctacctggatggcaaagggaacctggcacctcaaggatcgggtaaggggtgatgtaggaaacaggctctttggatgaattttctcccttaggttctgagggtggtgcctatgtgcccccgagtctgcgtctaacatgtgtttacccatgcctgccttgtgccatggtctgagtgggcgctgggctctgcatggagggctcagagttggagatgggggcccagacctgtaactagtcataatgcagcatgttggatgctaagacagaagtctgggcagcatgctggggcggtgtttcacccccagggtatgctgagcagagcttcacagagcctgaagctctcaggagtccgtctggcagagggtgggtggaagacaggacagagcacagaggtgtgcagagcctagatggtcagggctgagcaggctctaagagcagtctcttgccctggttgtcctgtcagaaaggcttcttgtggatgtgtgtggggatggtggttgagggggaggaggctggagaggccaggagagggccagctctccacctgtccctgcttcctgcctgtcctctggcagtgcccagtgctgtgtcttcactgacagatgaccttctcaagtactatcaccaggtgactcgtgctgtgctaggggatgatccgcaactgatgaaggttgcactccaggacttgcagacgaactccaagattggggcactcctgccttactttgtttatgtggtcagtggggtgaaatctgtaagccatgacctggagcaactgcaccggctgctgcaggtggcacggagcctatttcgtaatccgcacctgtgcttggggccctatgtccgctgtctggtgggcagtgtcctctactgtgtcctggagccac

SEQ ID NO: 499 - DNA, 678 bp, Homo sapiens
gagtgtgagctcgtgagtgggcgccgccgccaccgcccccgccgccgtcgtctcggtagcagccttcgccacgccggggtcttcagctccactggggccatgtcagagcgagaagagcggcggtttgtggagatccctcgggagtctgtccggctcatggcggagagcacgggcctggagctgagcgatgaggtggcggcgctgctcgcagaggacgtgtgctatcgtctgagagaggccacgcagaatagctctcagttcatgaagcacaccaaacgccggaagctgacggttgaggacttcaacagggccctcagatggagcagcgtggaggctgtgtgtggttacggatcacaggaggcactgcccatgcgccccgccagggagggtgaactctactttcctgaggatcgagaggtgaacctggtggagctggccctggctaccaacatccccaaaggctgtgctgagacagctgtcagagttcatgtctcctacctggatggcaaagggaacctggcacctcaaggatcggggtgaaatctgtaagccatgacctggagcaactgcaccggctgctgcaggtggcacggagcctatttcgtaatccgcacctgtgcttggggccctatgtccgctgtctggtgggcagtgtcctctactgtgtcctggagccac

SEQ ID NO: 500 - DNA, 780 bp, Homo sapiens
gagtgtgagctcgtgagtgggcgccgccgccaccgcccccgccgccgtcgtctcggtagcagccttcgccacgccggggtcttcagctccactggggccatgtcagagcgagaagagcggcggtttgtggagatccctcgggagtctgtccggctcatggcggagagcacgggcctggagctgagcgatgaggtggcggcgctgctcgcagaggacgtgtgctatcgtctgagagaggccacgcagaatagctctcagttcatgaagcacaccaaacgccggaagctgacggttgaggacttcaacagggccctcagatggagcagcgtggaggctgtgtgtggttacggatcacaggaggcactgcccatgcgccccgccagggagggtgaactctactttcctgaggatcgagagttcatgtctcctacctggatggcaaagggaacctggcacctcaaggatcggtgcccagtgctgtgtcttcactgacagatgaccttctcaagtactatcaccaggtgactcgtgctgtgctaggggatgatccgcaactgatgaaggttgcactccaggacttgcagacgaactccaagattggggcactcctgccttactttgtttatgtggtcagtggggtgaaatctgtaagccatgacctggagcaactgcaccggctgctgcaggtggcacggagcctatttcgtaatccgcacctgtgcttggggccctatgtccgctgtctggtgggcagtgtcctctactgtgtcctggagccac

SEQ ID NO: 501 - DNA, 300 bp, Homo sapiens
atttttgatatcctcgggaatgagcagccacaagcagggtcatacctcgtcagaatatgatatgcttcgggagatgttcagtgattctagaagtaacaatgatgatgatgaggatgaggatgatgaagatgaggatgaggatgaggatgaagatgaagacaaagaagaggaggaggaagattgttctgaagagtatctggaaaggcagctgcaggccgagtttattgaatctggccagtatagggcaaatgaaggtaccagttcaatagtcatggaaattcagaagcagattgagaaaaa

SEQ ID NO: 502 - DNA, 300 bp, Homo sapiens
ggacttcttgatgcagctggaagattacacgcctacggtgggcttccgcccgaacaaggccacctagcctgctgtcaaaactttcagccacatcgtgcttttcagcgttctcttccatttgctcccctagtcgctcttctgtgtttgccctctgctcacccaaactgtgagcttcctgataatcaggcctatccatttccctcaccctcctcccgctctgctgacagttctcttaattgatttctcagatcccagatgcagtgactggttactacctgaaccgtgctggctttgaggcct

SEQ ID NO: 503 - DNA, 300 bp, Homo sapiens
caatgatgccctacagcactgcaaaatgaagggcacggcctccggcagctcccggagcaagagcaaggtgtgaggggaggcttaatgaatcagtaattaccttccacaacagtggaggcttatcctgccacccctttcgggaaactgaatcgtaggggaggtgtaagacttactcagggtcacccatctgggattgaagtccgggattcctgtgctcagttggtgctcttccctcttccctcaggaccgcaagtacactctaaccatggaggacttgacccctgccctcagcgagtatgg

SEQ ID NO: 504 - DNA, 450 bp, Homo sapiens
ggacttcttgatgcagctggaagattacacgcctacggtgggcttccgcccgaacaaggccacctagcctgctgacaaaactttcagccacatcgtgcttttcagcgttctcttccatttgctcccctagtcgctcttctgtgtttgccctctgctcacccaaactgtgagcttcctgataatcaggcctatccatttccctcaccctcctcccgctctgctgacagttctcttaattgatttctcagatcccagatgcagtgactggttactacctgaaccgtgctggctttgaggcctcagacccacgcatgtgagtaaacccagggcaggttagttttgggtgcttgtgcagtatgttgtccatctccttctcatctaagttttttctctctagaattcggctcatctccttagctgcccagaaattcatctcagatattgccaatg

SEQ ID NO: 505 - DNA, 638 bp, Homo sapiens
ggccatatctaacggggtttacgtactgccgagcgcggccaacggagacgtgaagcccgtggtgtccagcacgcctttggtggacttcttgatgcagctggaagattacacgcctacggtgggcttccgcccgaacaaggccacctagcctgctgtcaaaactttcagccacatcgtgcttttcagcgttctcttccatttgctcccctagtcgctcttctgtgtttgccctctgctcacccaaactgtgagcttcctgataatcaggcctatccatttccctcaccctcctcccgctctgctgacagttctcttaattgatttctcagatcccagatgcagtgactggttactacctgaaccgtgctggctttgaggcctcagacccacgcataattcggctcatctccttagctgcccagaaattcatctcagatattgccaatgatgccctacagcactgcaaaatgaagggcacggcctccggcagctcccggagcaagagcaaggaccgcaagtacactctaaccatggaggacttgacccctgccctcagcgagtatggcatcaatgtgaagaagccgcactacttcacctgagccacccaacctaaatgtacttatctgtccccatgtccc

SEQ ID NO: 506 - DNA, 62 bp, Homo sapiens
gaaggaattcctgcaatcagtgcaatgagcctagaccagaggactctcgtccctcaggagga

SEQ ID NO: 507 - DNA, 75 bp, Homo sapiens
gaaacgactacagaaatgatcagcgcaaccgaccatactgatgactgttttgaatgttcctttgtctctgacatg

SEQ ID NO: 508 - DNA, 508 bp, Homo sapiens
ttgatgaccctccttcagctaaggcagccattgactggtttgatggaaaagaattccatggcaacatcattaaagtgtcctttgccactagaagacctgaattcatgagaggaggtggaagtggaggtgggcggcgaggccgtggaggatatagaggtcgtggaggctttcaagggagaggtggagaccccaaaagtggggattgggtttgccctaatccgtcatgcggaaatatgaactttgctcgaaggaattcctgcaatcagtgcaatgagcctagaccagaggactctcgtccctcaggaggagatttccgggggagaggctacggtggagagaggggctacagaggtcgtgggggcagaggtggagaccgaggtggctatggaggcaaaatgggaggaagaaacgactacagaaatgatcagcgcaaccgaccatactgatgactgttttgaatgttcctttgtctctgacatgatccatagtgaaattgccagagttttgc

SEQ ID NO: 509 - DNA, 70 bp, Homo sapiens
agattattgcatgtggcgtggttatgagtattgtcgactggatggacaaaccccgcatgaagaaagagag

SEQ ID NO: 510 - DNA, 75 bp, Homo sapiens
gaggaagcaatagaggcttttaatgctcctaatagtagcaaattcatctttatgctaagtaccagggctggaggt

SEQ ID NO: 511 - DNA, 86 bp, Homo sapiens
cccgctgagaaactgtcaccaaatccccccaaactgacaaagcagatgaacgctatcatcgatactgtgataaactacaaagatag

SEQ ID NO: 512 - DNA, 75 bp, Homo sapiens
ttcagggcgacagctcagtgaagtcttcattcagttaccttcaaggaaagaattaccagaatactatgaattaat

SEQ ID NO: 513 - DNA, 75 bp, Homo sapiens
ttcgaccagaagtcctccagccatgagcggcgcgccttcctgcaggccatcctggagcacgaggagcaggatgag

SEQ ID NO: 514 - DNA, 75 bp, Homo sapiens
gaggaagacgaggtgcccgacgacgagaccgtcaaccagatgatcgcccggcacgaggaggagtttgatctgttc

SEQ ID NO: 515 - DNA, 805 bp, Homo sapiens
gttttcccagcctcagtctctctttcgttttccttttcccttcccccaaccctccgcccttctctaaatcagccggccttccttgacctcagtgacccgtctggccccgcccaccctcgtcgacgtgattcccgccgtgaggaaatatttgatgatgcgtcacctggaaagcaaaaggaaatccaagaaccagatcctacctatgaagaaaaaatgcaaactgaccgggcaaatagattcgagtatttattaaagcagacagaactttttgcacatttcattcaacctgctgctcagaagactccaacttcacctttgaagatgaaaccagggcgcccacgaataaaaaaagatgagaagcagaacttactatccgttggcgattaccgacaccgtagaacagagcaagaggaggatgaagagctattaacagaaagctccaaagcaaccaatgtttgcactcgatttgaagactctccatcgtatgtaaaatggggtaaactgagagattatcaggtccgaggattaaactggctcatttctttgtatgagaatggcatcaatggtatccttgcagatgaaatgggcctaggaaagactcttcaaacaatttctcttcttgggtacatgaaacattatagaaacattcctgggcctcatatggttttggttcctaagtctacattacacaactggatgagtgaattcaagagatgggtaccaacacttagatctgtttgtttgataggagataaagaacaaagagctgcttttgtcagagacgttttattaccgggagaatggg

SEQ ID NO: 516 - DNA, 54 bp, Homo sapiens
aggcgactagccactgtggaagagaggaagaaaatagttgcatcgtcacatgat

SEQ ID NO: 517 - DNA, 75 bp, Homo sapiens
cacggatacacgactctagccaccagtgtgaccctgttaaaagcctcggaagtggaagagattctggatggcaac

SEQ ID NO: 518 - DNA, 29 bp, Homo sapiens
tgccaggcagcgggcacccaggcgtggcg

SEQ ID NO: 519 - DNA, 75 bp, Homo sapiens
gacccaggcacccccctgcctccagaccccacagccccgagcccaggcacggtcacccctgtgccacctccacag

SEQ ID NO: 520 - DNA, 104 bp, Homo sapiens
ttcccccccctggaccccatggcccctcaccgttccccaaccaacaaactcctccctcaatgatgccaggggcagtgccaggcagcgggcacccaggcgtggcg

SEQ ID NO: 521 - DNA, 75 bp, Homo sapiens
gcccaaagccctgccattgtggcagctgttcagggcaacctcctgcccagtgccagcccactgccagacccaggc

SEQ ID NO: 522 - DNA, 300 bp, Homo sapiens
atgctgagagtcgaccaaccccaatggggcctccgcctacctctcacttccatgtcttggctgacacaccatcagggctggtgcctctgcagcccaagacacctcagggccgccaggttgatgctgataccaaggctgggcgaaagggcaaagagctggatgacctggtgccagagacggctaagggcaagccagagctgcagacctctgcttcccaacaaatgctcaactttcctgacaaaggcaaagagaaaccaacagacatgcaaaactttgggctgcgcacagacatgtacacaa

SEQ ID NO: 523 - DNA, 762 bp, Homo sapiens
cacttggctgctgttgaggaaaggaagatcaaatctttggtggccctgctggtggagacccagatgaaaaagttggagatcaaacttcggcactttgaggagctggagactatcatggaccgggagcgagaagcactggagtatcagaggcagcagctcctggccgacagacaagccttccacatggagcagctgaagtatgcggagatgagggctcggcagcagcacttccaacagatgcaccaacagcagcagcagccaccaccagccctgcccccaggctcccagcctatccccccaacaggggctgctgggccacccgcagtccatggcttggctgtggctccagcctctgtagtccctgctcctgctggcagtggggcccctccaggaagtttgggcccttctgaacagattgggcaggcagggtcaactgcagggccacagcagcagcaaccagctggagccccccagcctggggcagtcccaccaggggttcccccccctggaccccatggcccctcaccgttccccaaccaacaaactcctccctcaatgatgccaggggcagtgccaggcagcgggcacccaggcgtggcggcccaaagccctgccattgtggcagctgttcagggcaacctcctgcccagtgccagcccactgccagacccaggcacccccctgcctccagaccccacagccccgagcccaggcacggtcacccctgtgccacctccacagtgaggagccagccagacatct

SEQ ID NO: 524 - DNA, 126 bp, Homo sapiens
gcccttggtgctgcaggcgcggtgggctccgggcccaggcaccgagggggcactggatgactctccaggtgcaggaccctgccatctatgactccaggtcttcagcacccacccaccgtggtacag

SEQ ID NO: 525 - DNA, 75 bp, Homo sapiens
cagtgccaagaggaggaagatggctgacaaaatcctccctcaaaggattcgggagctggtccccgagtcccaggc

SEQ ID NO: 526 - DNA, 126 bp, Homo sapiens; identical to SEQ ID NO: 524

SEQ ID NO: 527 - DNA, 75 bp, Homo sapiens
caaaagcggaagctgcgactctatatctccaacacttttaaccctgcgaagcctgatgctgaggattccgacggc

SEQ ID NO: 528 - DNA, 67 bp, Homo sapiens
acagctgaaaacagccctgtcacacctgttggagcccagaaaacagcactgcgaatttcacagagca

SEQ ID NO: 529 - DNA, 75 bp, Homo sapiens
gaatgattggtaacagtgcttctcggcctactatgccatctggagaatgggcaccgcagagttcggctgtgagag

SEQ ID NO: 530 - DNA, 852 bp, Homo sapiens
tagccagctctttgtcggatacaaacaaagactccacaggtagcttgcctggttctgggtctacacatggaacctcgctcaaggagaagcataaaattttgcacagactcttgcaggacagcagttcccctgtggacttggccaagttaacagcagaagccacaggcaaagacctgagccaggagtccagcagcacagctcctggatcagaagtgactattaaacaagagccggtgagccccaagaagaaagagaatgcactacttcgctatttgctagataaagatgatactaaagatattggtttaccagaaataacccccaaacttgagagactggacagtaagacagatcctgccagtaacacaaaattaatagcaatgaaaactgagaaggaggagatgagctttgagcctggtgaccaggaatgattggtaacagtgcttctcggcctactatgccatctggagaatgggcaccgcagagttcggctgtgagagtcacctgtgctgctaccaccagtgccatgaaccggccagtccaaggaggtatgattcggaacccagcagccagcatccccatgaggcccagcagccagcctggccaaagacagacgcttcagtctcaggtcatgaatatagggccatctgaattagagatgaacatggggggacctcagtatagccaacaacaagctcctccaaatcagactgccccatggcctgaaagcatcctgcctatagaccaggcgtcttttgccagccaaaacaggcagccatttggcagttctccagatgacttgctatgtccacatcctgcagctgagtctccgagtgatgagggagctctcct

SEQ ID NO: 531 - DNA, 828 bp, Homo sapiens
tagccagctctttgtcggatacaaacaaagactccacaggtagcttgcctggttctgggtctacacatggaacctcgctcaaggagaagcataaaattttgcacagactcttgcaggacagcagttcccctgtggacttggccaagttaacagcagaagccacaggcaaagacctgagccaggagtccagcagcacagctcctggatcagaagtgactattaaacaagagccggtgagccccaagaagaaagagaatgcactacttcgctatttgctagataaagatgatactaaagatattggtttaccagaaataacccccaaacttgagagactggacagtaagacagatcctgccagtaacacaaaattaatagcaatgaaaactgagaaggaggagatgagctttgagcctggtgaccagcctggcagtgagctggacaacttggaggagattttggatgatttgcagaagtcacctgtgctgctaccaccagtgccatgaaccggccagtccaaggaggtatgattcggaacccagcagccagcatccccatgaggcccagcagccagcctggccaaagacagacgcttcagtctcaggtcatgaatatagggccatctgaattagagatgaacatggggggacctcagtatagccaacaacaagctcctccaaatcagactgccccatggcctgaaagcatcctgcctatagaccaggcgtcttttgccagccaaaacaggcagccatttggcagttctccagatgacttgctatgtccacatcctgcagctgagtctccgagtgatgagggagctctcct

SEQ ID NO: 532 - DNA, 104 bp, Homo sapiens
ggctccttggaagcaaacctgccagtggttatcaagctccttacatacccagcaccgacccccaggactggcttacccaaaagcagaccttggagaacagtcag

SEQ ID NO: 533 - DNA, 75 bp, Homo sapiens
gaagtattacttaattcacctctacaggaggaacataacttccccccagaccattatggcctccctgcagtttgt

SEQ ID NO: 534 - DNA, 125 bp, Homo sapiens
gcagcctgtcagctctccgggtcggaatcctatggttcaacagggaaatgtgccacctaacttcatggtgatgcagcagcaaccaccaaaccaggggccacagagtttacatccaggcctaggag

SEQ ID NO: 535 - DNA, 75 bp, Homo sapiens
agcaggacaggccaatccgaactttatgcaaggtcaggtgccttcgaccacagcaaccacccctgggaattcagg

SEQ ID NO: 536 - DNA, 64 bp, Homo sapiens
tttgattgtgtattatggataccaaggaagagaagaaggaacggaaacaaagttattttgctcg

SEQ ID NO: 537 - DNA, 75 bp, Homo sapiens
agatgacaatcaaaacaaaacacatgataaaaaagagaagaagatggtggttcagaagccccatgggactatgga

SEQ ID NO: 538 - DNA, 1748 bp, Homo sapiens
cagcaatctctgagaccaaggttaagaagagacatgttgaccctttcatggaatggactcagatcatcaccaagtacttatgggagcagttacagaagatggctgaatactaccggccagggcctgcaggaagtgggggctgtggttccacgatagggcccttgccccatgatgtagaggtggcaatccggcagtgggattacaccgagaagctggccatgttcatgtttcaggatggaatgctggacagacatgagttcctgacctgggtgcttgagtgttttgagaagatccgccctggagaggatgaattgcttaaactgctgctgcctctgcttctccgatactctggggaatttgttcagtctgcatacctgtcccgccggcttgcctacttctgtacacggagactggccctgcagctggatggtgtgagcagtcactcatctcatgttatatctgctcagtcaacaagcacgctacccaccacccctgctcctcagcccccaactagcagcacaccctcgactccctttagtgacctgcttatgtgccctcagcaccggcccctggtttttggcctcagctgtatcctacagaccatcctcctgtgctgtcctagtgccttggtttggcactactcactgactgatagcagaattaagaccggctcaccacttgaccacttgcctattgccccgtccaacctgcccatgccagagggtaacagtgccttcactcagcaggtatgtctgaccactagcctggtactctcagattgggctatgaggctaaattactctttcagaagtagtgatttggagtctagtactattcttctagcctggggctctggccttttatatgccttggtacatccttgtagccttcctttttaacattgcaggtccgtgcaaagttgcgggagatcgagcagcagatcaaggagcggggacaggcagttgaagttcgctggtctttcgataaatgccaggaagctactgcaggcttcaccattggacgggtacttcatactttggaagtgctggacagccatagttttgaacgctctgacttcagcaactctcttgactccctttgtaaccgaatctttggattgggacctagcaaggatgggcatgagatctcctcagatgatgatgctgtggtgtcattgctatgtgaatgggctgtcagctgcaagcgttctggtcggcatcgtgctatggtggtagccaagctcctggagaagagacaggcggagattgaggctgaggttagagggcagagataagagaacaagattggccaatgggaaggaatttactgcggttggagaccgagagatggaggtggtggagggaccagagttgaaggtgtgagaacagagtaaagaagcaaaagagaacctaaaggcaaagttacggacgtgaggcgaaagtagagaagagtggattgtagtaagagttagagataacatcaaggcttcagttgggaggtggtaaagaacatggaggtcagcaggggaatgaaagtgaaaagcatggggtagaggtcaagcaggtggtagtttaaggcctacacattgaggagtgaagaagcaggtaaaagtcagttctacaatttgttctgtcatcttgcagcgttgtggagaatcagaagccgcagatgagaagggttccatcgcctctggctccctttctgctcccagtgctcccattttccaggatgtcctcctgcagtttctg

SEQ ID NO: 539 - DNA, 2326 bp, Homo sapiens
cagcaatctctgagaccaaggttaagaagagacatgttgaccctttcatggaatggactcagatcatcaccaagtacttatgggagcagttacagaagatggctgaatactaccggccagggcctgcaggaagtgggggctgtggttccacgatagggcccttgccccatgatgtagaggtggcaatccggcagtgggattacaccgagaagctggccatgttcatgtttcaggatggaatgctggacagacatgagttcctgacctgggtgcttgagtgttttgagaagatccgccctggagaggatgaattgcttaaactgctgctgcctctgcttctccgatactctggggaatttgttcagtctgcatacctgtcccgccggcttgcctacttctgtacacggagactggccctgcagctggatggtgtgagcagtcactcatctcatgttatatctgctcagtcaacaagcacgctacccaccacccctgctcctcagcccccaactagcagcacaccctcgactccctttagtgacctgcttatgtgccctcagcaccggcccctggtttttggcctcagctgtatcctacagaccatcctcctgtgctgtcctagtgccttggtttggcactactcactgactgatagcagaattaagaccggctcaccacttgaccacttgcctattgccccgtccaacctgcccatgccagagggtaacagtgccttcactcagcaggtccgtgcaaagttgcgggagatcgagcagcagatcaaggagcggggacaggcagttgaagttcgctggtctttcgataaatgccaggaagctactgcaggcttcaccattggacgggtacttcatactttggaagtgctggacagccatagttttgaacgctctgacttcagcaactctcttgactccctttgtaaccgaatctttggattgggacctagcaaggatgggcatgagatctcctcagatgatgatgctgtggtgtcattgctatgtgaatgggctgtcagctgcaagcgttctggtcggcatcgtgctatggtggtagccaagctcctggagaagagacaggcggagattgaggctgagcgttgtggagaatcagaagccgcagatgagaagggttccatcgcctctggctccctttctgctcccagtgctcccattttccaggatgtcctcctgcagtttctggatacacaggctcccatgctgacggaccctcgaagtgagagtgagcgggtggaattctttaacttagtactgctgttctgtgaactgattcgacatgatgttttctcccacaacatgtatacttgcactctcatctcccgaggggaccttgcctttggagcccctggtccccggcctccctctccctttgatgatcctgccgatgacccagagcacaaggaggctgaaggcagcagcagcagcaagctggaagatccagggctctcagaatctatggacattgaccctagttccagtgttctctttgaggacatggagaagcctgatttctcattgttctcccctactatgccctgtgaggggaagggcagtccatcccctgagaagccagatgtcgagaaggaggtgaagcccccacccaaggagaagattgaagggacccttggggttctttacgaccagccacgacacgtgcagtacgccacccattttcccatcccccaggaggagtcatgcagccatgagtgcaaccagcggttggtcgtactgtttggggtgggaaagcagcgagatgatgcccgccatgccatcaagaaaatcaccaaggatatcttgaaggttctgaaccgcaaagggacagcagaaactgaccagcttgctcctattgtgcctctgaatcctggagacctgacattcttaggtggggaggatgggcagaagcggcgacgcaaccggcctgaagccttccccactgctgaagatatctttgctaagttccagcacctttcacattatgaccaacaccaggtcacggctcaggtgtgggcctaagcccagcccctttcccacattctggcctcctgttctgttttccttttcttccctatcttctccctgctaggcaggctaagcctcctggtctcatccccttccagtgtcatcctttcctccttccctggttctttcctctctccactcccatctcactcccactgcccttatcaggtctcccggaatgttctggagcagatcacgagctttgcccttggcatgtcataccacttgcctctggtgcagcatgtgcagttcatcttcgacctcatgga

SEQ ID NO: 540 - DNA, 128 bp, Homo sapiens
tgatgatgctgtggtgtcattgctatgtgaatgggctgtcagctgcaagcgttctggtcggcatcgtgctatggtggtagccaagctcctctggtgcagcatgtgcagttcatcttcgacctcatgga

SEQ ID NO: 541 - DNA, 2236 bp, Homo sapiens
tgatgatgctgtggtgtcattgctatgtgaatgggctgtcagctgcaagcgttctggtcggcatcgtgctatggtggtagccaagctcctggagaagagacaggcggagattgaggctgagcgttgtggagaatcagaagccgcagatgagaagggttccatcgcctctggctccctttctgctcccagtgctcccattttccaggatgtcctcctgcagtttctggatacacaggctcccatgctgacggaccctcgaagtgagagtgagcgggtggaattctttaacttagtactgctgttctgtgaactgattcgacatgatgttttctcccacaacatgtatacttgcactctcatctcccgaggggaccttgcctttggagcccctggtccccggcctccctctccctttgatgatcctgccgatgacccagagcacaaggaggctgaaggcagcagcagcagcaagctggaagatccagggctctcagaatctatggacattgaccctagttccagtgttctctttgaggacatggagaagcctgatttctcattgttctcccctactatgccctgtgaggggaagggcagtccatcccctgagaagccagatgtcgagaaggaggtgaagcccccacccaaggagaagattgaagggacccttggggttctttacgaccagccacgacacgtgcagtacgccacccattttcccatcccccaggaggagtcatgcagccatgagtgcaaccagcggttggtcgtactgtttggggtgggaaagcagcgagatgatgcccgccatgccatcaagaaaatcaccaaggatatcttgaaggttctgaaccgcaaagggacagcagaaactgaccagcttgctcctattgtgcctctgaatcctggagacctgacattcttaggtggggaggatgggcagaagcggcgacgcaaccggcctgaagccttccccactgctgaagatatctttgctaagttccagcacctttcacattatgaccaacaccaggtcacggctcaggtctcccggaatgttctggagcagatcacgagctttgcccttggcatgtcataccacttgcctctggtgcagcatgtgcagttcatcttcgacctcatggaatattcactcagcatcagtggcctcatcgactttgccattcagctgctgaatgaactgagtgtagttgaggctgagctgcttctcaaatcctcggatctggtgggcagctacactactagcctgtgcctgtgcatcgtggctgtcctgcggcactatcatgcctgcctcatcctcaaccaggaccagatggcacaggtctttgaggggctgtgtggcgtcgtgaagcatgggatgaaccggtccgatggctcctctgcagagcgctgtatccttgcttatctctatgatctgtacacctcctgtagccatttaaagaacaaatttggggagctcttcaggtaagagaggtggaaggtaaggggtagcgagtgggacctactcccttcttcccatgaccacccaactcaggaggagaggatggcccgggaccctgctgcctgtctagggtcatttgtggactgtgtcctccacatactgttgtgttaccaagagtgggccctcttcctcagcaggcttgctccccgcctatatctgtggggcccaccctcttcccccttttcctcactgccttcagaggccccagttccttattcccatgtggttcctttcctgcccagtctgttttgtcccatctcccttttcttgtctcaagatccttcatccctcactttctcctttttttcttttctcccctttcctgaccatccctcgacctcagcaggccttcttcaacactactatctcctttcctccatccctgcagcgacttttgctcaaaggtgaagaacaccatctactgcaacgtggagccatcggaatcaaatatgcgctgggcacctgagttcatgatcgacactctagagaaccctgcagctcacaccttcacctacacggggctagtagggtgaatgacatcgcaatcctgtgtgcagagctgaccggctattgcaagtcactgagtgcagaatggctaggagtgcttaaggccttgtgctgctcctctaacaatggcacttgtggtttcaacgatctcctctgcaatgttgatgtcagtgacctatcttttcatgactcgctggctacttttgt

SEQ ID NO: 542 - DNA, 2324 bp, Homo sapiens
tgatgatgctgtggtgtcattgctatgtgaatgggctgtcagctgcaagcgttctggtcggcatcgtgctatggtggtagccaagctcctggagaagagacaggcggagattgaggctgagcgttgtggagaatcagaagccgcagatgagaagggttccatcgcctctggctccctttctgctcccagtgctcccattttccaggatgtcctcctgcagtttctggatacacaggctcccatgctgacggaccctcgaagtgagagtgagcgggtggaattctttaacttagtactgctgttctgtgaactgattcgacatgatgttttctcccacaacatgtatacttgcactctcatctcccgaggggaccttgcctttggagcccctggtccccggcctccctctccctttgatgatcctgccgatgacccagagcacaaggaggctgaaggcagcagcagcagcaagctggaagatccagggctctcagaatctatggacattgaccctagttccagtgttctctttgaggacatggagaagcctgatttctcattgttctcccctactatgccctgtgaggggaagggcagtccatcccctgagaagccagatgtcgagaaggaggtgaagcccccacccaaggagaagattgaagggacccttggggttctttacgaccagccacgacacgtgcagtacgccacccattttcccatcccccaggaggagtcatgcagccatgagtgcaaccagcggttggtcgtactgtttggggtgggaaagcagcgagatgatgcccgccatgccatcaagaaaatcaccaaggatatcttgaaggttctgaaccgcaaagggacagcagaaactgaccagcttgctcctattgtgcctctgaatcctggagacctgacattcttaggtggggaggatgggcagaagcggcgacgcaaccggcctgaagccttccccactgctgaagatatctttgctaagttccagcacctttcacattatgaccaacaccaggtcacggctcaggtctcccggaatgttctggagcagatcacgagctttgcccttggcatgtcataccacttgcctctggtgcagcatgtgcagttcatcttcgacctcatggaatattcactcagcatcagtggcctcatcgactttgccattcagctgctgaatgaactgagtgtagttgaggctgagctgcttctcaaatcctcggatctggtgggcagctacactactagcctgtgcctgtgcatcgtggctgtcctgcggcactatcatgcctgcctcatcctcaaccaggaccagatggcacaggtctttgaggggctgtgtggcgtcgtgaagcatgggatgaaccggtccgatggctcctctgcagagcgctgtatccttgcttatctctatgatctgtacacctcctgtagccatttaaagaacaaatttggggagctcttcaggtaagagaggtggaaggtaaggggtagcgagtgggacctactcccttcttcccatgaccacccaactcaggaggagaggatggcccgggaccctgctgcctgtctagggtcatttgtggactgtgtcctccacatactgttgtgttaccaagagtgggccctcttcctcagcaggcttgctccccgcctatatctgtggggcccaccctcttcccccttttcctcactgccttcagaggccccagttccttattcccatgtggttcctttcctgcccagtctgttttgtcccatctcccttttcttgtctcaagatccttcatccctcactttctcctttttttcttttctcccctttcctgaccatccctcgacctcagcaggccttcttcaacactactatctcctttcctccatccctgcagcgacttttgctcaaaggtgaagaacaccatctactgcaacgtggagccatcggaatcaaatatgcgctgggcacctgagttcatgatcgacactctagagaaccctgcagctcacaccttcacctacacggggctaggcaagagtcttagtgagaaccctgctaaccgctacagctttgtctgcaatgcccttatgcacgtctgtgtggggcaccatgatcccgatagggtgaatgacatcgcaatcctgtgtgcagagctgaccggctattgcaagtcactgagtgcagaatggctaggagtgcttaaggccttgtgctgctcctctaacaatggcacttgtggtttcaacgatctcctctgcaatgttgatgtcagtgacctatcttttcatgactcgctggctacttttgt

SEQ ID NO: 543 - DNA, 1621 bp, Homo sapiens
tgatgatgctgtggtgtcattgctatgtgaatgggctgtcagctgcaagcgttctggtcggcatcgtgctatggtggtagccaagctccacttgcctctggtgcagcatgtgcagttcatcttcgacctcatggaatattcactcagcatcagtggcctcatcgactttgccattcaggtggggaagttggggagatgagggtggaggcaggagttcatgccatatagcggctacggagggtcataaggacaggcgtagaggctccagccagtttcccaagcatctgctgaccctcccaaccttgcttcttcatgcaggctgtgtggcgtcgtgaagcatgggatgaaccggtccgatggctcctctgcagagcgctgtatccttgcttatctctatgatctgtacacctcctgtagccatttaaagaacaaatttggggagctcttcaggtaagagaggtggaaggtaaggggtagcgagtgggacctactcccttcttcccatgaccacccaactcaggaggagaggatggcccgggaccctgctgcctgtctagggtcatttgtggactgtgtcctccacatactgttgtgttaccaagagtgggccctcttcctcagcaggcttgctccccgcctatatctgtggggcccaccctcttcccccttttcctcactgccttcagaggccccagttccttattcccatgtggttcctttcctgcccagtctgttttgtcccatctcccttttcttgtctcaagatccttcatccctcactttctcctttttttcttttctcccctttcctgaccatccctcgacctcagcaggccttcttcaacactactatctcctttcctccatccctgcagcgacttttgctcaaaggtgaagaacaccatctactgcaacgtggagccatcggaatcaaatatgcgctgggcacctgagttcatgatcgacactctagagaaccctgcagctcacaccttcacctacacggggctaggcaagagtcttagtgagaaccctgctaaccgctacagctttgtctgcaatgcccttatgcacgtctgtgtggggcaccatgatcccgagtatggggtgtactgagtgaggaagggcaccatgcccccatctgagatagggagggctgaggtacccgggaggtactacaaccttgattatttagtggggcagagatgagaagttaatgggtctgaggttttgtggagcaaggtttttcctgagggcatttgtacttttccctagtagggtgaatgacatcgcaatcctgtgtgcagagctgaccggctattgcaagtcactgagtgcagaatggctaggagtgcttaaggccttgtgctgctcctctaacaatggcacttgtggtttcaacgatctcctctgcaatgttgatgtgagacttggggtggggttttgctagtggggcagtgaccagggcagggggctggttgtgatcctctgaccagggacagagttccgtagagtggaggcacaccgctttgagtgggcctccacactgagtcatggtgtctgtctgttttttcctccaggtcagtgacctatcttttcatgactcgctggctacttttgt

SEQ ID NO: 544 - DNA, 1504 bp, Homo sapiens
gcagctcacaccttcacctacacggggctaggcaagagtcttagtgagaaccctgctaaccgctacagctttgtctgcaatgcccttatgcacgtctgtgtggggcaccatgatcccgatagggtgaatgacatcgcaatcctgtgtgcagagctgaccggctattgcaagtcactgagtgcagaatggctaggagtgcttaaggccttgtgctgctcctctaacaatggcacttgtggtttcaacgatctcctctgcaatgttgatgtcagtgacctatcttttcatgactcgctggctacttttgttgccatcctcatcgctcggcagtgtttgctcctggaagatctgattcgctgtgctgccatcccttcactccttaatgctggtgaactaccaatctgtaacccctagcatttctagacctcaaatttcaatacacactggacggccatcctctcattgttcactgtgggagaccttgctgcggctccctggccttcctcagaaggccagtcctttggtatgctgaaggctagaagaaacctgttttttagccctggatttgcagccctgacctttccaatttctgacccttcaactgcgtaacagttctctgctctacctcgctttcaatattatcttgctttttctcctttcactttacctcatcttctctcccatgcccctgccatacacttgcatgcatgcaggcacgcacacacataaacccacatacagtttaacttcatcccttccagatctgttttgtcttccttttagcttgtagtgaacaggactctgagccaggggcccggcttacctgccgcatcctccttcaccttttcaagacaccgcagctcaatccttgccagtctgatggaaacaagcctacagtaggaatccgctcctcctgcgaccgccacctgctggctgcctcccagaaccgcatcgtggatggagccgtgtttgctgttctcaaggctgtgtttgtacttggggatgcggaactgaaaggttcaggcttcactgtgacaggaggaacagaagaacttccagaggaggagggaggaggtggcagtggtggtcggaggcagggtggccgcaacatctctgtggagacagccagtctggatgtctatgccaagtacgtgctgcgcagcatctgccaacaggaatgggtaggagaacgttgccttaagtctctgtgtgaggacagcaatgacctgcaagacccagtgttgagtagtgcccaggcgcagcgcctcatgcagctcatttgctatccacatcgactgctggacaatgaggatggggaaaacccccagcggcagcgcataaagcgcattctccagaacttggaccagtggaccatgcgccagtcttccttggagctgcagctcatgatcaagcagacccctaacaatgagatgaactccctcttggagaacatcgccaaggccacaatcgaggttttccaacggtcagcagagacagggtcatc

SEQ ID NO: 545 - DNA, 697 bp, Homo sapiens
cataggcctgtacacccagaaccagccactacctgcaggtggccctcgtgtggacccataccgtcctgtgcgcttaccaatgcagaagctgcccacccgaccaacttaccctggagtgctgcccacaaccatgactggcgtcatgggtttagaaccctcctcttataagacctctgtgtaccggcagcagcaacctgcggtgccccaaggacagcgccttcgccaacagctccaggcaaagatagtgagaggggcagtagggagggctgtcagggagaggggcttttgagggtcacaggacggaggagacacttgggatcttcacaaggacactcagggtgggagacacaagagatgagatggcagcaagcatttcctgagtttgagttgttctcttttctccctttagcagagtcagggcatgttgggacagtcatctgtccatcagatgactcccagctcttcctacggtttgcagacttcccagggctatactccttatgtttctcatgtgggattgcagcaacacacaggccctgcaggtaccatggtgccccccagctactccagccagccttaccagagcacccacccttctaccaatcctactcttgtagatcctacccgccacctgcaacagcggcccagtggctatgtgcaccagcaggcccccacctatggacatggactgacctcc

SEQ ID NO: 546 - DNA, 622 bp, Homo sapiens
cataggcctgtacacccagaaccagccactacctgcaggtggccctcgtgtggacccataccgtcctgtgcgcttaccaatgcagaagctgcccacccgaccaacttaccctggagtgctgcccacaaccatgactggcgtcatgggtttagaaccctcctcttataagacctctgtgtaccggcagcagcaacctgcggtgccccaaggacagcgccttcgccaacagctccaggcaaagatagtgagaggggcagtagggagggctgtcagggagaggggcttttgagggtcacaggacggaggagacacttgggatcttcacaaggacactcagggtgggagacacaagagatgagatggcagcaagcatttcctgagtttgagttgttctcttttctccctttagcagagtcagggcatgttgggacagtcatctgtccatcagatgactcccagctcttcctacggtttgcagacttcccagggctatactccttatgtttctcatgtgggattgcagcaacacacaggccctgcagatcctacccgccacctgcaacagcggcccagtggctatgtgcaccagcaggcccccacctatggacatggactgacctcc

SEQ ID NO: 547 - DNA, 1128 bp, Homo sapiens
cttgctcctattgtgcctctgaatcctggagacctgacattcttaggtggggaggatgggcagaagcggcgacgcaaccggcctgaagccttccccactgctgaagatatctttgctaagttccagcacctttcacattatgaccaacaccaggtcacggctcaggtctcccggaatgttctggagcagatcacgagctttgcccttggcatgtcataccacttgcctctggtgcagcatgtgcagttcatcttcgacctcatggaatattcactcagcatcagtggcctcatcgactttgccattcagctgctgaatgaactgagtgtagttgaggctgagctgcttctcaaatcctcggatctggtgggcagctacactactagcctgtgcctgtgcatcgtggctgtcctgcggcactatcatgcctgcctcatcctcaaccaggaccagatggcacaggtctttgaggggtaagcagagcttcggaataactgaaacaaagctctggcgaatgccggtggaagtggcctgggaagagcatgcacttcctcacactctggggaagcacctgctgctcaggctgtgtggcgtcgtgaagcatgggatgaaccggtccgatggctcctctgcagagcgctgtatccttgcttatctctatgatctgtacacctcctgtagccatttaaagaacaaatttggggagctcttcagcgacttttgctcaaaggtgaagaacaccatctactgcaacgtggagccatcggaatcaaatatgcgctgggcacctgagttcatgatcgacactctagagaaccctgcagctcacaccttcacctacacggggctaggcaagagtcttagtgagaaccctgctaaccgctacagctttgtctgcaatgcccttatgcacgtctgtgtggggcaccatgatcccgatagggtgaatgacatcgcaatcctgtgtgcagagctgaccggctattgcaagtcactgagtgcagaatggctaggagtgcttaaggccttgtgctgctcctctaacaatggcacttgtggtttcaacgatctcctctgcaatgttgatgtcagtgacctatcttttcatgactcgctggctacttttgt

SEQ ID NO: 548 - DNA, 985 bp, Homo sapiens
ccacctagaactggattgtgcgctggccgccaccgctgccacctgctcagagtgaaataatgaaggtggtcaacctgaagcaagccattttgcaagcctggaaggagcgctggagttactaccaatgggcaatcaacatgaagaaattctttcctaaaggagccacctgggatattctcaacctggcagatgcgttactagagcaggccatgattggaccatcccccaatcctctcatcttgtcctacctgaagtatgccattagttcccagatggtgtcctactcttctgtcctcacagccatcagtaagtttgatgacttttctcgggacctgtgtgtccaggcattgctggacatcatggacatgttttgtgaccgtctgagctgtcacggcaaagcagaggaatgcatcggactgtgccgagcccttcttagcgccctccactggctgctgcgctgcacggcagcctctgcagagcggctgcgggaggggctggaggccggcactccagccgctggggagaagcagcttgccatgtgccttcagcgcctggagaaaaccctcagcagcaccaagaaccgggccctgctgcacatcgccaaactagaggaggcctcattgcacacatcccagggacttgggcagggtggcacccgagccaatcaaccaacagcttcttggactgccatcgagcattctctcttgaaacttggagagatcctgaccaatctcagcaacccgcagctccggagtcaggccgagcagtgtggcaccctcattaggagcatccccacgatgctgtctgtgcatgcggagcagatgcacaagaccggcttccccactgtccacgccgtgatcctgctcgagggcaccatgaacctgacaggcgagacgcagtccctggtggagcagctgacgatggtgaagcgcatgcagcatatccccaccccactttttgtcctggagatctggaaagcttgctt

SEQ ID NO: 549 - DNA, 1300 bp, Homo sapiens
ccacctagaactggattgtgcgctggccgccaccgctgccacctgctcagagtgaaataatgaaggtggtcaacctgaagcaagccattttgcaagcctggaaggagcgctggagttactaccaatgggcaatcaacatgaagaaattctttcctaaaggagccacctgggatattctcaacctggcagatgcgttactagagcaggccatgattggaccatcccccaatcctctcatcttgtcctacctgaagtatgccattagttcccagatggtgtcctactcttctgtcctcacagccatcagtaagtttgatgacttttctcgggacctgtgtgtccaggcattgctggacatcatggacatgttttgtgaccgtctgagctgtcacggcaaagcagaggaatgcatcggactgtgccgagcccttcttagcgccctccactggctgctgcgctgcacggcagcctctgcagagcggctgcgggaggggctggaggccggcactccagccgctggggagaagcagcttgccatgtgccttcagcgcctggagaaaaccctcagcagcaccaagaaccgggccctgctgcacatcgccaaactagaggaggcctcattgcacacatcccagggacttgggcagggtggcacccgagccaatcaaccaacagccactggattctggcctccctctgcctctctctcctgagcctgtgtgatgccataccttctgaagtcagctggctgtgtcccctggaaatcaggcttttgggaatggtctctggggtttccagctctaggtgcccaccccccttctggaaacagtgcatgctgccctcaggcccctccctccctgttgtcctcaggggaagccttcctgtgtggtttcgtgtgccggagggagtgccaaaatcgaggagttcagggccaggtgctccttctctcctgtttcccatcatgtttctgtacttccttccctctgccagcttcttggactgccatcgagcattctctcttgaaacttggagagatcctgaccaatctcagcaacccgcagctccggagtcaggccgagcagtgtggcaccctcattaggagcatccccacgatgctgtctgtgcatgcggagcagatgcacaagaccggcttccccactgtccacgccgtgatcctgctcgagggcaccatgaacctgacaggcgagacgcagtccctggtggagcagctgacgatggtgaagcgcatgcagcatatccccaccccactttttgtcctggagatctggaaagcttgctt

SEQ ID NO: 550 - DNA, 548 bp, Homo sapiens
ggaacaggagtttcgttccattttccagcacatacaatcagctcagtctcagcgtagcccctcagaactgtttgcccaacatatagtgaccattgttcaccatgttaaagagcatcactttgggtcctcaggaatgacattacatgaacgctttactaaatacctaaagagaggaactgagcaggaggcagccaaaaacaagaaaagcccagagatacacaggagaatagacatttcccccagtacattcagaaaacatggtttggctcatgatgaaatgaaaagtccccgggaacctggctacaaggatgggcataattctaaaaatgaactacaaagggttaatttttattaaatgtatcaacaacctttgtgaagtggttagaatatggtaaatgaccccaaagtctattgaggtgagcttgagaaaaaaaagagaggagttttggaacaagtgcccatgatgagagaagaaactttttgtgatatttttctgcttgctgagggaaaatacaaagatgatcctgttgatctccgccttgatattg

SEQ ID NO: 551 - DNA, 824 bp, Homo sapiens
acggagaagatccaggagaagaagatcaagaaagaagactcgagctctgggctcatgaacactctcctgaatggacacaagggtggggactgcgatggcttctccaccttcgatgttcccatcttcactgaagagttcttggaccaaaacaaaggcacgggcgaaacgcccacgctgggcactctggacttctacatggcccggcttcacggagccatcgagcgcgaccccgcccagcacgagaagctcatcgtccgcatcaaggaaatcctggcccaggtcgccagcgagcacctgtgaggagtgggcgggcccacgatgcagaggagaagctgtgggcgcggccctgccacaccccaccccgtggacgagaggctgggggtccaccctttggggcctggtcccatcctgcacctttgggggctccagcccccctaaaattaaatttctgcagcatccctttagctttcaatctccccagccccctgaacccggaaaaagcactcgctgcgcgatacacccagaagaacctcacagccgagggtgcccctcctcggaggacagccacgcgctacactggctctccgggccacccccaggacacagggcagacgaaacccacccccagcacacggcaggaccccccaaattactcactacggggggctgtgccataggccacacaggaagctgccttgtggggacttacctggggtgtcccccgcatgcctgtaccccagatgggtgggggccggctttgcccatcctgctctcctccagccgagggaccctggtgggggtggctccttctcactgctggatcc

SEQ ID NO: 552 - DNA, 566 bp, Homo sapiens
caggggaaggctgaacgtgctggccaacgtgatccgcaaggacctggagcagatcttctgccagtttgaccccaagctggaggcggcggacgagggctccggggatgtcaagtaccacctgggcatgtaccacgagaggatcaaccgcgtcaccaaccggaacatcactctgtcgctggttgccaacccctcccacctggaggcagtggaccctgtggtgcaggggaagacaaaggcagagcagttctaccgtggagatgcccagggcaagaagcccctcctggctcacacctgccctgcaggtcatgtccatcctggttcatggggacgccgcctttgctggccagggcgtggtatatgagaccttccacctgagcgacctgccctcctacacgaccaatggtaccgtgcacgtcgtcgtcaacaaccagattggattcaccacagacccccgaatggcccgctcctcaccatacccgaccgacgtggcccgggtggtcaatgcgcctatcttccatgtgaatgccgatgacccaaaggctgtgatatatgtgtgcagtgtggca

SEQ ID NO: 553 - DNA, 210 bp, Homo sapiens
gacgagtccggttcgtgttcgtccgcggagatctctctcatctcgctcggctgcgggaaatcgggctgaagcgactgagtccgcgatggagagagaaaaggaacagttccgtaagctctttattggtggcttaagctttgaaaccacagaagaaagtttgaggaactactacgaacaatggggaaagcttacagactgtgtggtaatgag

SEQ ID NO: 554 - DNA, 210 bp, Homo sapiens
gcgaaggaaggcaccaaggagaaatcaggacccacctctctgcctctgggcaaactgttttggaaaaagtcagttaaagaggactcagtccccacaggtgcggaggagaatacatcagactccacagaaaagactatcacaccgccagagcctgaaccaacaggagcaccacagaagggtaaagagggctcctcgaaggacaagaagtca

SEQ ID NO: 555 - DNA, 770 bp, Homo sapiens
gcagcagcaccaggctctgcagcggcaacccccagcggcttaagccatggcgtgagtaccggggcgggtcgtccagctgtgctcctggggccggcgcgggttttggattggtggggtgcggcctggggccagggcggtgccgccaagggggaagcgatttaacgagcgcccgggacgcgtggtctttgcttgggtgtccccgagacgctcgcgtgcctgggatcgggaaagcgtagtcgggtgcccggactgcttccccaggagccctacagccctcggaccccgagccccgcaaggtcccaggggtcttggctgttgccccacgaaacgtgcaggaaccaagatggcggcggcagggcggcggcgcgggcgtgagtcaagggcgggcggtgggcggggcgcggccgctggccgtatttggacgtggggacggagcgctttcctcttggcggccggtggaagaatcccctggtctccgtgagcgtccattttgtggaacctgagttgcaagcagggaggggcaaatacaactgccctgttcccgattctctagatggccgatctagagaagtcccgcctcataagtggaaggatgaaattctcagaacagctaacctctaatgggagttggcttctgattctcattcaggcttctcacggcattcagcagcagcgttgctgtaaccgacaaagacaccttcgaattaagcacattcctcgattccagcaaagcaccgcaacatgaccgaaatgagcttcctgagcagcga

SEQ ID NO: 556 - DNA, 140 bp, Homo sapiens
gccatcttgcgtccccgcgtgtgtgcgcctaatctcaggtggtccacccgagaccccttgagcaccaaccctagtcccccgcgcggccccttattcgctccgacaagatgaaagaaacaatcatgaaccaggaaaaactc

SEQ ID NO: 557 - DNA, 181 bp, Homo sapiens
ggtccgccgacatggcctggaccaagtaccagctgttcctggccgggctcatgcttgttaccggctccatcaacacgctctcggcaaagcagtgggcatgttcctgggagaattctcctgcctggctgccttctacctcctccgatgcagagctgcagggcaatcagactccagcgtagac

SEQ ID NO: 558 - DNA, 180 bp, Homo sapiens
cctggagcgcaagttccgtcagaaacagtacctctccattgcagagcgtgcagagttctccagctctctgaacctcacagagacccaggtcaaaatctggttccagaacagaaggtaaagccatgttttgacttggtgaaaatggggttgtcaaacagcccattaagctccctggtattt

SEQ ID NO: 559 - DNA, 240 bp, Homo sapiens
ggcatctcgtccccggtgaagaagacagagatggacaagtcaccattcaacagcacgtcccctgcaaaccgttcctttgtgggattaggaccaagggatcctgcgggcatttatcaggcacagtcctggtatctgggatagcaaaggtcttcttccctcgccccttctccatcgtcccaggaatcccagggggcagcacagccggcccccggcccacgttttcggtggaaaattagagtg

SEQ ID NO: 560 - DNA, 210 bp, Homo sapiens
cgtgcccccaacactgccgagctcaagatctgccgagtgaaccgaaactctggcagctgcctcggtggggatgagatcttcctactgtgtgacaaggtgcagaaagaggacattgaggtgtgtccccaagccagcaccccagccctatccctttacgtcatccctgagcaccatcaactatgatgagtttcccaccatggtgtttccttc

SEQ ID NO: 561 - DNA, 180 bp, Homo sapiens
acagcgagctgcaggactctaatccagagtttaccttccagcagccctacgaccaggcccacctgctggcagccatcccacgaggtgtgactaactatgcaataatccacccccaggtgcagccccagggcctgcggaggcggtggcagactagagtctgagatgccccgagcccaggca

SEQ ID NO: 562 - DNA, 280 bp, Homo sapiens
tgtcagcaactcctgcccagctgagctgcccaacatcaaacgggagatctctgagaccgaggcaaaggcccttttgaaggaacggcagaagaaagacaatcacaacctaattgagcgtcgcaggcgattcaacattaacgacaggatgttgctccatcctttgtcttggaaccaccagtctagtccgtcctggcacagaagaggagtcaagtaatggaggtcccagccctgggggtttaagctctgccccttccccatgaaccctgccctgctctgccca

SEQ ID NO: 563 - DNA, 210 bp, Homo sapiens
ttacaccttttctactgtacaccccatcccagacgaagacagtccctggatcaccgacagcacagacagaatccctgctaccaatatggactccagtcatagtacaacgcttcagcctactgcaaatccaaacacaggtttggtggaagatttggacaggacaggacctctttcaatgacaacgcagcagagtaattctcagagcttctc

SEQ ID NO: 564 - DNA, 490 bp, Homo sapiens
agccgccttcccggggccagtttccttccctctcagccagggatgcctcgagcagccacaggggcagggtgagtggcgggccgctaggggccgcggctgcctctgcccactgcacccactgcacagaaaccgtggggagggagcatggagcctcacagggccccgtggggagggagcatggagcctcacagggccttgaagagctgtgccccagggggagctgcgtgtgcgggtctgtgaatgcgcacacacgtgtaacacgtgccccgcacggagccgtcctggcccctcagcctctcctgctgtcctggtctgtggaatgtgggcccgggccctgctgggctgagggcaacaggagtcacgtggaagaggtgccacacacgcgtccacaggcggggctcctctgctcagattctccgagtgtgccgaacgtcctgactgccatcctgctgctgctgcgggagctggatgcagaggggctggaggccgt

SEQ ID NO: 565 - DNA, 420 bp, Homo sapiens
tgctgcccctgggggcatgaagagccccccagaccagcccgtcaagcacctcttcaccacaggtgtggtctacgacacgttcatgctaaagcaccagtgcatgtgcgggaacacacacgtgcaccctgagcatgctggccggatccagagcatctggtcccggctgcaggagacaggcctgcttagcaagtgcgagcggatccgaggtcgcaaagccacgctagatgagatccagacagtgcactctgaataccacaccctgctctatgggaccagtcccctcaaccggcagaagctagacagcaagaagttgctcggccccatcagccagaagatgtatgctgtgctgccttgtgggggcatcggggtggacagtgacaccgtgtggaatgagatgcactcctccagtgctgtgcgcat

SEQ ID NO: 566 - DNA, 232 bp, Homo sapiens
  misc_feature (21)..(21): n is a, c, g, or t
gtttagtgtcttttccttgtntctgctcggggagcgtgaggcagatcggccggctttgctccaggcctcaggagtgtcactcgcctnggcttgcacagtacattggaacgtgcgggttctattttgtattcgacgtgccggatcgaaatagagctcgcggcactntgaagaccacagtaggaagttaaggacgggggtgcaggttcgcagccctatcaaccagctccgagcc

SEQ ID NO: 567 - DNA, 180 bp, Homo sapiens
gatgtgaaggtggacactgaggatatggagaagaaaccagagtcatttttcactcaattcgatgctatgggatttttccttgggtggctgcattctttgaaacaccaaaggaacacatttctctgtgtgtctgacttgctgctccagggatgtcatagttaaagttgaccagatctgtca

SEQ ID NO: 568 - DNA, 280 bp, Homo sapiens
tcctgtgcagtgggcatcaagtacatgggtgtgttcacgtacgtgctcgtgctgggtgttgcagctgtccatgcctggcacctgcttggagaccagactttgtccaatgtaggtgctgatgtccagtgctgcatgaggccggcctgtatggggcagatgcggatgtcacagggggtctgtgtgttctgtcacttgctcgcccgagcagtggctttgctggtcatcccggtcgtcctgtacttactgttcttctacgtccacttgattctagtcttccgct

SEQ ID NO: 569 - DNA, 210 bp, Homo sapiens
ggctgcgtttctgtgggaggccctgaaacgcgcggagcttccctctgcctccaggctttcccagcgagagtgaaattaaacttgaaactcggatcaactggcagtcgttgttggtattgttgcagcatctggcagtgagactgaggatgaggacagcatggacattcccttggacctttcttcatccgctggctcaggcaagagaaggag

SEQ ID NO: 570 - DNA, 180 bp, Homo sapiens
cctgttcagcctgccttctccacggtgccgttctcccagcctgtctgtttcccacccaggcccagggggcgcagacaaaaaacccagacagtcatccacacagtgcagagcgcccctggacagatgttctctactcccgccatcccacctatgatgtacccccaccccgcctatccgatg

SEQ ID NO: 571 - DNA, 180 bp, Homo sapiens
tggtaggaagagaaagaaacggaccagcatcgagaccaacatccgcctgactctggagaagaggtttcaagatgtatctccctcagggtctctgggccccctctctgtccctcctgtccacagtaccatgcctggaacagtaacgtcatcctgttcccctgggaacaacagcaggccttc

SEQ ID NO: 572 - DNA, 180 bp, Homo sapiens
ggggatgggggctgcagctcgtctgagcgcccctcgagcgctggtactctgggctgcactgggggcagcagctcacatcggaccatcacctatcagggctctctcagcaccccgccctgctccgagactgtcacctggatcctcattgaccgggccctcaatatcacctcccttcagatg

SEQ ID NO: 573 - DNA, 360 bp, Homo sapiens
acccgggacttcacccagctcaacgagctgcaatgccgctttcccaggcgcctggtggtccttggcttcccttgcaaccaatttggacatcaggagagacagaagtagcaaaccctctttcgagatgtccctccagccccagaagtacctccagcctcacaccatctcttcagcctagcaagttgctggagggagtctataacctaccaggagccagccagccattgtatcaagaaatagaaatctgccaggtacagggctcacacctataatcccagcgcttgggaggctaaggagaacagtcagaatgaggagatcctgaacagtctcaagtatgtccgtcctgggggtggataccag

SEQ ID NO: 574 - DNA, 662 bp, Homo sapiens
ccacatcaaagacagctttcacagtttgcgggactcagtcccatcactccaaggagagaagctctatttcctcttttggaaattgtgtactcctgtccttcatcgtcaaagtttgatgcagaaatgccacaccttcatttcaagctaccaagtgcacaagaaaaaagaatgcaagatttaaaaaatgattgttttgaccccttacacaaatgtcttactcctggctttaattaagctgcttgagggctgatagctctgccttaccctggtaatcagcaaaatggtcctgtggctggggaggccctggcagcaggaagccttcaaggagccatgggtctgtgctgactctggccttacaaccttccagcctcctttgctggcattgatggggttccatttttgaatgaactagtttaatgtggatccaaatttattgtgcatattctttcgttttggttttcaaaagatggcttattcacatggaaatgtacaccagtttagccctgggccctccctttaccttcatatgtgtaaaagcttacacaggtttcagaaaataaatggtttcattttctctaaaataactagtacaaaataaaacagatgtcagttgttgaaaaaaaaaaaaaaaaaaaaaaaaaaaaaaaaaaaaaaaaaaaaaa

SEQ ID NO: 575 - DNA, 140 bp, Homo sapiens
ccagaagcctgcatttctgcattctgcttaattccctttccttagatttgaaagaagccaacactaaaccacaaatatacaacaaggccattttctcaaacgagagtcagcctttaacgaaatgaccatggttgacacag

SEQ ID NO: 576 - DNA, 199 bp, Homo sapiens
gtgaccatgacagtaatgaaaccagggtcccaaccaagaaatctaactcaaacgtccacttcatttgttccattcctgattcttgggtaataaagacaaactttgtacctctcaaaaaaaaaaaaaaaaaagttggcctgcaggcggccgcaggtaagccagcccaggcctcgccctccagctcaaggcgggacagggc

SEQ ID NO: 577 - DNA, 1620 bp, Homo sapiens
gcaatcaaagaattaaaactacaaacaaaccatgttacaatgctgctaagaggaggaagatgatgatgttgatggtgacgtcaatgttgagaaaaatgaaactgaaccaccaaaaggaaaaaagaaaaaacaaaagaataaacagctgcagaagcctcagaaaaataagcccttacttgtagatgttgatctcagcttgtcagcatatgccaatgccaaaaagtattatgatcacaagagatatgctgctaagaaaacacaaaagactgttgaagctgctgagaaggcattcaagtcagcagaaaagaaaacaaagcaaacattaaaagaagttcagactgttacctctattcaaaaagcaagaaaagtatattgcttaggattcagcttcttaagtctgatcacagccgggcgcagtggctcacgcctgtaatcccagcactttgggaggccgaggagggcggatcacgaggtcaagagatcgagaccatcctggctaacacggtggacgagatcagcaacagaatgaaataattgtgaaaagatacttgacaccaggagacatttatgtacatgctgatcttcatggagctactagctgtgtaattaagaatccaacaggagaacccatccccccacggaccttgactgaagctggcacaatggcactttgctacagtgctgcttgggatgcacgagttatcactagtgcttggtgggtgtaccatcatcaggtatctaaaacagcaccaactggagaatatttgacaacaggaagcttcatgataagaggaaaaaagaattttcttcctccctcatatctaatgatggggtttagcttcctttttaaggtagatgagtcttgtgtttggagacatcagggtgaacgaaaagtcagagtacaggatgaagacatggagacactggcaagttgtacaagtgaactcatatcagaagaaatggaacaattagatggaggtgacacgagcagtgatgaggataaagaagaacatgaaactcctgtggaagtagaactcatgactcaggttgaccaagaggatatcactcttcagagtggcagagatgaactaaatgaggagctcattcaggaagaaagctctgaagacgaaggagaatatgaagaggttagaaaagatcaggattctgttggtgaaatgaaggatgaaggggaagagacattaaattatcctgatactaccattgacttgtctcaccttcaaccccaaaggtccatccagaaattggcttcaaaagaggaatcttctaattctagtgacagtaaatcacagagccggagacatttgtcagccaaggaaagaagtagagatggggtttcaccgtgttgggcaggattgtctcgatcttctgacctcgcgatccacccgccttggcctcccaaagtgctggattacagtcaaccaaccggtcaacagatgttttattgaatgcctaagacctgccaatgctatgttggtacaaagactacaaatcccagtgcctggccatcaagggaaatgaaaaagaaaaaacttccaagtgactcaggagatttagaagcgttagagggaaaggataaagaaaaagaaagtactgtacaca

SEQ ID NO: 578 - DNA, 179 bp, Homo sapiens
gctggagatattgacatagagttgtggtccaaagaagctcctaaagcttgcagaaattttatccaactttgtttggaagcttattatgacaataccatttttcatagagttgtgcctggtttcatagtccaaggcggagatcctactggcacagggagtggtggagagtctatctatgg

SEQ ID NO: 579 - DNA, 360 bp, Homo sapiens
caggagctgacacagaagatacagcaaatggaggcccagcatgacaaaactgaaaatgaacagtatttgttgctgacctcccagaatacatttttgacaaagttaaaggaagaatgctgtacattagccaagaaactggaacaaatctctcaaaaaaccagatctgaaatagctcaactcagtcaagaaaaaaggtatacatatgataaattgggaaagttacagagaagaaatgaagaattggaggaacagtgtgtccagcatgggagagtacatgagacgatgaagcaaaggctaaggcagctggataagcacagccaggccacagcccagcagctggtgcagctcctcagcaagcag

SEQ ID NO: 580 - DNA, 300 bp, Homo sapiens
ggctggaggaaagggaactgaacgcggttctgggagcagcaagcccacgggtagcagccgaggccccagaatgagtacaaggaatgcttctccctgtatgacaagcagcagagggggaagataaaagccaccgacctcatggtggccatgaggtgcctgggggccagcccgacgccaggggaggtgcagcggcacctgcagacccacgggatagacggaaatggagagctggatttctccacttttctgaccattatgcacatgcaaataaaacaagaagacccaaagaaagaaattctt

SEQ ID NO: 581 - DNA, 480 bp, Homo sapiens
cagaatcgccagacagaagtgcctgtcaaagtgctgtttgtggcccacaatcctcaacatggaaacttcctatcctgcctagggatcacagctgggccagaagctgggcttacagagattctctaaaggcagaagaaaacagaaaattgcaaaagatgaaggatgaacaacatcaaaagagtgaattactggaactgaaacggcagcagcaagagcaagaaagagccaaaatccaccagactgaacacaggagggtaaataatgcttttctggaccgactccaaggcaaaagtcaaccaggtggcctcgagcaatctggaggctgttggaatatgaatagcggtaacagctggggttctctattagttttttcgaggcacctaagggtatatgagaaaatattgactcctatctggccttcatcaactgacctcgaaaagcctcatgagatgctttttcttaatgtgattttgttcagcc

SEQ ID NO: 582 - DNA, 420 bp, Homo sapiens
agagacacacgcggagaggaggagaggctgagggagggaggtggagaaggacgggagaggcagagagaggagacacgcagagacactcaggaggggagagacaccgagacgcagagacactcaggaggggagagacaccgagacgcagagacacccaggccggggagcgcgagggagcgaggcacagacctggcccagcccgggcgccgaccctcctcccgctcccgcgccctcccctcggcgggcacggtatttttatccgtgcgcgaacagccctcctcctcctctcgccgcacagccaccaacgcctgccatgctgttccggctctcagagcactcctcaccagctgtgcagcgcattgctgagtctcacctgcagtctatcagcaatttgaatgagaaccaggcctcagaggagga

SEQ ID NO: 583 - DNA, 180 bp, Homo sapiens
gtgggattggagataggggaccagattgtcgaagtcaatggcgtcgacttctctaacctggatcacaaggagggccgggagctgttcatgacagaccgggagcggctggcagaggcgcggcagcgtgagctgcagcggcaggagcttctcatgcagaagcggctggcgatggagtccaac

SEQ ID NO: 584 - DNA, 180 bp, Homo sapiens
ctgatccccgtgaaaagctctcctgatgagcccctcacttggcagtatgtggatcagtttgtgtcggaatctgggggcgtgcgaggcagcctgggctcccctggaaatcgggaaaacaaggagaagaaggtcttcatcagcctggtaggctcccgaggccttggctgcagcatttccagc

SEQ ID NO: 585 - DNA, 2539 bp, Homo sapiens
gtttacaaacacgggctcccggcaggtgcgcgccgccccgcccgtgcgcggccggggttcgagggtggctcccgcgggcctcggggtgcccggacgggggctgcggtgctggctgcgtgcccgcttcttccatgccgtcctggggcaccggaaaatccgccgccaggcgctgtccccgacacgggctgtcgcctggttgggcccggaaatgggacgtcgcgctttctcagggagcgtagaagcagccagggcctctccaagccgctgctgtgacagaaagtgagtgagctgccggaggatgtccaccgccacgacagtcgcccccgcggggatcccggcgaccccgggccctgtgaacccaccccccccggaggtctccaaccccagcaagcccggccgcaagaccaaccagctgcagtacatgcagaatgtggtggtgaagacgctctggaaacaccagttcgcctggcccttctaccagcccgtggacgcaatcaaattgaacctgccggattatcataaaataattaaaaacccaatggatatggggactattaagaagagactagaaaataattattattggagtgcaagcgaatgtatgcaggacttcaacaccatgtttacaaattgttacatttataacaagcccacagatgacatagtgctaatggcccaagctttagagaaaatttttctacaaaaagtggcccagatgccccaagaggaagttgaattattaccccctgctccaaagggcaaaggtcggaagccggctgcgggagcccagagcgcaggtacacagcaagtggcggccgtgtcctctgtctccccagcgaccccctttcagagcgtgccccccaccgtctcccagacgcccgtcatcgctgccacccctgtaccaaccatcactgcaaacgtcacgtcggtcccagtccccccagctgccgccccacctcctcctgccacacccatcgtccccgtggtccctcctacgccgcctgtcgtcaagaaaaagggcgtgaagcggaaagcagacacaaccactcccacgacgtcggccatcactgccagccggagtgagtcgcccccgccgttgtcagaccccaagcaggccaaagtggtggcccggcgggagagtggtggccgccccatcaagcctcccaagaaggacctggaggacggcgaggtgccccagcacgcaggcaagaagggcaagctgtcggagcacctgcgctactgcgacagcatcctcagggagatgctatccaagaagcacgcggcctacgcctggcccttctacaagccagtggatgccgaggccctggagctgcacgactaccacgacatcatcaagcacccgatggacctcagcaccgtgaaaaggaagatggatggccgagagtacccagacgcacagggctttgctgctgatgtccggctgatgttctcgaattgctacaaatacaatcccccagaccacgaggttgtggccatggcccggaagctccaggacgtgtttgagatgaggtttgccaagatgccagatgagcccgtggaggcaccggcgctgcctgcccccgcggcccccatggtgagcaagggcgctgagagcagccgtagcagtgaggagagctcttcggactcaggcagctcggactcggaggaggagcgggccaccaggctggcggagctgcaggagcagctgaaggccgtgcacgagcagctggccgccctgtctcaggccccagtaaacaaaccaaagaagaagaaggagaagaaggagaaggagaagaagaagaaggacaaggagaaggagaaggagaagcacaaagtgaaggccgaggaagagaagaaggccaaggtggctccgcctgccaagcaggctcagcagaagaaggctcctgccaagaaggccaacagcacgaccacggccggcagagatcatttcttgacctgtggagtttgagacgcctatggggtgtagagaggaacgaacctctgtaattgtttcctggccaagggctggaaaccccgcagctgggagcgacttttctaaccttggattttctgccttggggcaccactttgggaagaaagcttggtcccagagagcagcctgctgttgggaggaaggggtgtgtgcagtgggctcccacggcaggtagacggagactcaacaccacgttgctctgtctcctgccccagacagctgaagaaaggcggcaagcaggcatctgcctcctacgactcagaggaagaggaggagggcctgcccatgagctacgatgaaaagcgccagcttagcctggacatcaaccggctgcccggggagaagctgggccgggtagtgcacatcatccaatctcgggagccctcgctcagggactccaaccccgacgagatagaaattgactttgagactctgaaacccccccctttgcgggaactggagagatatgtcaagtcttgtttacagaaaaagcaaaggaaaccgttctgtaaaaaaaaaaaaaaaaaaaaaaaaaaaaaaaaaaaa

SEQ ID NO: 586 - DNA, 720 bp, Homo sapiens
aagagctcgttgattcctctgcaaggtggtgcagcatcctctgtcccttcattcatttcagatctactcaggtctccctgtaaacagatctctcggatcaataagcatgaatgacgaagactacagcaccatctatgacacaatccaaaatgagaggacgtatgaggttccagaccagccagaagaaaatgaaagtccccattatgatgatgtccatgagtacttaaggccagaaaatgatttatatgccactcagctgaatacccatgagtatgattttgtgtcagtctataccattaagggtgaagagaccagcttggcctctgtccagtcagaagacagaggctacctcctgcctgatgagatatactctgaactccaggaggctcatccaggtgagccccaggaggacaggggcatctcaatggaagggttatattcatcaacccaggaccagcaactctgcgcagcagaactccaggagaatgggagtgtgatgaaggaagatctgccttctccttcaagcttcaccattcagcacagtaaggccttctctaccaccaagtattcctgctattctgatgctgaaggtttggaagaaaaggagggagctcacatgaaccctgagatttacctctttgtgaaggctggaatcgatggagaaagcatcggcaactgtcctttctctcagcgcctcttcatgatcctctggctgaaagg

SEQ ID NO: 587 - DNA, 1560 bp, Homo sapiens
gttgagtcaatgtgtccccctcttgttcctagggtgcgggcttcatggccttctcctccaggaagctccacctgatcatgtcctgggtggatatccagcccccatagttcagggcctactagcagctgctagatcttgaactccaggagcgccccacgccttgggagcttggcatgggctaaatactcccccatttgttaaatggggtcctgaaacctgaccagggaagacgggataaagtagccatgggtcatcgcagcccctttgaagccgggcctggccacccaaaggcaactcaggggtggagactgaggcctcaggagaagcccccactagaatgctctctgcccctcccttccagattaaccaaaacctgctaattgtggaagccctcggcatgctcccctcccccacagcctcttcctcccttccctcccctcccccttccatccgaatgataaaggccccagcccgcctgccccagcccggcctcaggtcccggccctgccttctacactgccccaccgccctgcaccctccacccggccaggcccctgcccacgctgtctaccgtcccgcatggggccctgcagcggctcccgcctggggcccccagaggcagagtcgccctcccagccccctaagaggaggaagaagaggtacctgcgacatgacaagcccccctacacctacttggccatgatcgccttggtgattcaggccgctccctcccgcagactgaagctggcccagatcatccgtcaggtccaggccgtgttccccttcttcagggaagactacgagggctggaaagactccattcgccacaacctttcctccaaccgatgcttccgcaaggtgcccaaggaccctgcaaagccccaggccaagggcaacttctgggcggtcgacgtgagcctgatcccagctgaggcgctccggctgcagaacaccgccctgtgccggcgctggcagaacggaggtgcgcgtggagccttcgccaaggacctgggcccctacgtgctgcacggccggccataccggccgcccagtcccccgccaccacccagtgagggcttcagcatcaagtccctgctaggagggtccggggagggggcaccctggccggggctagctccacagagcagcccagttcctgcaggcacagggaacagtggggaggaggcggtgcccaccccaccccttccctcttctgagaggcctctgtggcccctctgcccccttcctggccccacgagagtggagggggagactgtgcaggggggagccatcgggccctcaaccctctccccagagcctagggcctggcctctccacttactgcagggcaccgcagttcctgggggacggtccagcgggggacacagggcctccctctgggggcagctgcccacctcctacttgcctatctacactcccaatgtggtaatgcccttggcaccaccacccacctcctgtccccagtgtccgtcaaccagccctgcctactggggggtggcccctgaaacccgagggcccccagggctgctctgcgatcta

SEQ ID NO: 588 - DNA, 180 bp, Homo sapiens
tgtcttggctgacacaccatcagggctggtgcctctgcagcccaagacacctcagcagacctctgcttcccaacaaatgctcaactttcctgacaaaggcaaagagaaaccaacagacatgcaaaactttgggctgcgcacagacatgtacacaaaaaagaatgttccctccaagagcaa

SEQ ID NO: 589 - DNA, 179 bp, Homo sapiens
tcagttcctgcagtaccacgtcctcagcgactccaaacctttggcttgtctgctgttatccctagagagtttctatcctcctgctcatcagctatctctggacatgctgaagcgactttcaacagcaaatgatgaaatagtagaagttctcctttccaaacaccaagtgttagctgcct

SEQ ID NO: 590 - DNA, 660 bp, Homo sapiens
gaaaatgctggcacctgggcccagaagccagggcctctaactcctggggttgatttcttcagtgaagttgcaccttacaaagggaatatggccaaagcggcactcaactgaaggctgatatcaggcgattagacagccatgcattctgcgtttgtctggaatggattgtagagagatggacttatatgaggactaccagtccccgtttgattttgatgcaggagtgaacaaaagctatctctacttgtctcctagtggaaattcatctccacccggatcacctactcttcagaaatttggtctgctgagaacagacccagtccctgaggaaggagaagagaacttgcaaaggtagaagaagaaatccagactctgtctcaagtgttagcagcaaaagagaagcatctagcagagatcaagcggaaacttggaatcaattctctacaggaactaaaacagaacattgccaaagggtggcaagacgtgacagcaacatctgcgaggagcaagcttctagcagcagaaaccgaactgctctgtcttctgtattgagagccatctgcagagctgttacaagaagacatctgaaaccttatcccaggctggacagaaggcctcagctgctttttcgtctgttggctcagtcatcaccaaaaagct

SEQ ID NO: 591 - DNA, 120 bp, Homo sapiens
gaggaaatggaaacagatgctcgctcgtcccgtggctctgattccccagcagctgatgttgagattgagtatgtgactgaagaacctgaaatttacgagcccaactttatcttctttaag

SEQ ID NO: 592 - DNA, 180 bp, Homo sapiens
atgtcttcaaggctcctgctccccgcccttcattactgggactggacttgctggcttccctgaaacggagagagcggcagcagtgggaagatgaccagaggcaagccgatcgggattggtacatgatggacgagggctatgacgagttccacaacccgctggcctactcctccgaggact

SEQ ID NO: 593 - DNA, 240 bp, Homo sapiens
gggaaaaaaacagaatggaaagagtaaaaaagttgaagaggcagagcctgaagaatttgtcgtggaaaaagtactagatcgacgtgtagtgaatgggaaagtggaatatttcctgaagtggaagggaaagctggcaaagaaaaagatggtacaaaaagaaaatctttatctgacagtgaatctgatgacagcaaatcaaagaagaaaagagatgctgctgacaaaccaagaggatttgcc

SEQ ID NO: 594 - DNA, 180 bp, Homo sapiens
tcactctggaggcgactagccactgtggaagagaggaagaaaatagttgcatcgtcacatgatcacggatacacgactctagccaccagtgtgaccctgttaaaagcctcggaagtggaagagattctggatggcaacgatgagaagtacaaggctgtgtccatcagcacagagcccccc

SEQ ID NO: 595 - DNA, 179 bp, Homo sapiens
ggaaagtagacccatggcaatgggacctcctcctactcctcattttaatgtattagctgataccccctctgggcttgtgcctctgcatcttcgatcacctcagagtaaggtgctagtgctggaagagaatggactgaacaggagacccttctactcctggaggccctggagatgtacaa

SEQ ID NO: 596 - DNA, 210 bp, Homo sapiens
aagcctcgaatgggcgaaagttcacttagaaactttacaatagatctgtttgtttgataggagataaagaacaaagagctgcttttgtcagagacgttttattaccgggagaatggtatactcggatattaatgaaggatatagatatactcaactcagcaggcaagatggacaaaatgaggttattgaacatcctaatgcagttgagaa

SEQ ID NO: 597 - DNA, 300 bp, Homo sapiens
agagagcgggacttcaggcggcggaggcagcaccgaggaagcatttatgaccttctacagtgaggaataaagatggcatatagcataccagagattcattccaactagcattccaactctgacagtgacaccaagaatgttttcctgggactgcctggtgcttgttctccctggcattgtcttcaggtgaaacaaatagagaagagagactcggttctaacttcgaaaaatcagattgaaagactgacccgtcctggttcctcttacttcaatttgaacccatttgaggttcttcagata

SEQ ID NO: 598 - DNA, 180 bp, Homo sapiens
  misc_feature (109)..(109): n is a, c, g, or t
gaggtatttccaatccccgtcgaggtcaagatcaagatccaggtctatttcacgaccaagaagcagtcgttccccatcaggaagtcctcgcagaagtgcaagtcctganaagaatggactgaaagcttctcagttcacccttttaggggaaaagttatttttggttacattattataaag

SEQ ID NO: 599 - DNA, 180 bp, Homo sapiens
gcagctggcaggacctgaaggatcacatgcgagaagctggggatgtctgttatgctgatgtgcagaaggatggagtggggatggtcgagtatctcagaaaagaagacatgagggtgaaacttcctacatccgagtttatcctgagagaagcaccagctatggctactcacggtctcggtc

SEQ ID NO: 600 - DNA, 180 bp, Homo sapiens
ttgttttctttttttaatgaaactagatcactgcttacaaaaccctgcacaagccctcctgcccatccccttcacagttcccttggtgagacgggcaatgacacggcaagcggcatcgtgctggtacagagcgtgtgacagctcttggcgggttgtctgcagctgctggcgcagagtgaa

SEQ ID NO: 601 - DNA, 480 bp, Homo sapiens
ccccccatctcaggtgagaatctgattggcctgagcagagcccggcgcccccacaatgccatctttgtcaactttgaggatgaggaggtgcccaagcagcctatggattcgatttgggtatgacccccggaaaaacccagatgccaagatttatcaagtcctcgatttccgaatccgttgtggaatgaaacacggttacgcccccagtgacttgccggtcaaagcaaagcgcagcacctacaactacagcctccccatcaccgtcaagaagacatccagccagcttgtcaccatgcatgacctgaagcagggcctgggcccgtcggggacgagtggtgctcggaaaccagcttccagcaagtacaagctcaaggtcagccttcagacactgagggactctgtctacatcttccgggaaggggccttgccaccctatcggcagatgttctaccagttatgcgacttgaatgtggaagagtt

SEQ ID NO: 602 - DNA, 240 bp, Homo sapiens
cggaaatgctgacctgacctttgaccagacggcgtggggggacagtggtgtgtattactgctccgtggtctcagcccaggacctccaggggaacaatgaggcctacgcagagctcatcgtccttgtgtatgccgccggcaaagcagccacctcaggtgttcccagcatttatgcccccagcacctatgcccacctgtctcccgccaagaccccacccccaccagctatgattcccatggg

SEQ ID NO: 603 - DNA, 150 bp, Homo sapiens
tccgcccgccacgcagactggcgcgtccaggtggccgtgaagcacctgcacatccacactccgctgctcgacagaaaactgaatatcctgatgttgcttggccattgagatttcgcatcctgcatgaaattgcccttggtgtaaattacc

SEQ ID NO: 604 - DNA, 300 bp, Homo sapiens
gactcaccagatacaagagttaactcttgacacaccatactacttcaaaatccaggcacggaactcaaagggcatgggacccatgtctgaagctgtccaattcagaacacctaaagcctcagggtctggagggaaaggaagccggctgccagacctaggatccgactacaaacctccaatgagcggcagtaacagccctcatgggagccccacctctcctctggacagtaatatgctgctggtcataattgtttctgttggcgtcatcaccatcgtggtggttgtgattatcgctgtctt

SEQ ID NO: 605 - DNA, 225 bp, Homo sapiens
gcagacggacgactcgcttattcacttctgctggaaggacaggacgtccgggaacgtggaagacgacttgatcatcttccctgacgactgaacccaagacagaccaggatgaggagcattgccggaaagtcaacgagtatctgaacaaccccccgatgcctggggcgctgggggccagcggaagcagcggccacgaactctctgcgctaggcggtgagggtggcc

SEQ ID NO: 606 - DNA, 180 bp, Homo sapiens
aagtttataccaagtcttctcatttaaaagctcacctgaggactcacactgtgtgaagttatcagtaccagactattttgcttcaatctgcaaaaggaaggtgtgtgaaggtgaaaagccatacaagtgtacctgggaaggctgcgactggaggttcgcgcgatcggatgagctgacccg

SEQ ID NO: 607 - DNA, 150 bp, Homo sapiens
ccgcgcgcctgggagacgctgcctcggcccggacgcgcccgcgcccccgcggctggagggtggtcaacaacggttccagcctcagggatgagtgcatcacaaacctactggtgtttggcttcctccaaagctgttctgacaacagcttcc

SEQ ID NO: 608 - DNA, 250 bp, Homo sapiens
agtggcagctgacatgttttctgacggcaacttcaactggggccgggttgtcgcccttttctactttgccagcaaactggtgctcaaggctggcgtgaaatggcgtgatctgggctcactgcaacctctgcctcctgggttcaagcgattcacctgcctcagcatcccaaggagctgggattacaggccctgtgcaccaaggtgccggaactgatcagaaccatcatgggctggacattggacttcctcc

SEQ ID NO: 609 - DNA, 150 bp, Homo sapiens
accagaggttctcagaccggaaacacccagaccagtggacattggttctggaggatttggtgatgtcgagcagaaagaccatgggtttgaggtggcctccacttcccctgaagacgagtcccctggcagtaaccccgagccagatgccac

SEQ ID NO: 610 - DNA, 150 bp, Homo sapiens
tgcagcacctgcagcccacggcagagaatgcctatgagtacttcaccaagattgccaccaggccagcagcaacacccacagcctgtttgagagtggcatcaattggggccgtgtggtggctcttctgggcttcggctaccgtctggccct

SEQ ID NO: 611 - DNA, 152 bp, Homo sapiens
ctgcggtaccggcgggcattcagtgacctgacatcccagctccacatcaccccagggacagcatatcagagctttgaacaggatacttttgtggaactctatgggaacaatgcagcagccgagagccgaaagggccaggaacgcttcaaccg

SEQ ID NO: 612 - DNA, 200 bp, Homo sapiens
ggaaatgagggagctcatccaggccaaagtgggcagtttcagccagaatgtggaactcctcaacttgctgcctaagaggggtccccaagcttttgatgccttctgtgaagccttgcactcctgaattttatcaaacacacttccagctggcatataggttgcagtctcggcctcgtggcctagcactggtgttgagcaat

SEQ ID NO: 613 - DNA, 180 bp, Homo sapiens
agaagctgagatgtttggatggagctttgtctttgaggactttgtctctgatgagctgagaaacaaagccacccagccaatgagcctgcaggtcctggctctggcatccgagagagactggagcacccagtgttacacgtgagctggaatgacgcccgtgcctactgtgcttggcgggga

SEQ ID NO: 614 - DNA, 210 bp, Homo sapiens
gtcttttgcttagtgtcaatgcccgaggactcttggagtttgagcatcagagggcccctagggtctccccctcgtccctgccccctctggattggagcagacagctctcctaccttccaggcaaggatcaaaagacccagctgagggcgatggggcccagcctgaggaaacacccagggatggcgacaagccagaggagactcaggggaa

SEQ ID NO: 615 - DNA, 180 bp, Homo sapiens
cttatgtggtaaccaagacaaaagcgattaatgggaaataccatcgtttcttgggtcgtcatttcccccgcttctatatcctgtacacaatcttcatgaaagaaagccttgagccgggccatgcttctcacatcttacctgcctcctcccttgttgagacatcgtttgaagactcataca

SEQ ID NO: 616 - DNA, 240 bp, Homo sapiens
tctggagaaggatcagatgaacttacgcagggttacatatattttcacaaggattggagagggagaaagaaaaactgctttgtgtgccaaaagcaaaactcttggtgtttttgtttgtgaaataggctccttctcctgaaaaagccgaggaggagagtgagaggcttctgagggaactctatttgtttgatgttctccgcgcagatcgaactactgctgcccatggtcttgaactgagag

SEQ ID NO: 617 - DNA, 577 bp, Homo sapiens
atggcggaacaggctaccaagtccgtgctgtttgtgtgtctgggtaacatttgtcgatcacccattgcagaagcagttttcaggaaacttgtaaccgatcaaaacatctcagagaattggagggtagacagcgcggcaacttccggtgggtcattgatagcggtgctgtttctgactggaacgtgggccggtccccagaccaagagctgtggagctgcctaagaaatcatggcattcacacagcccataaagcaagacagattaccaaagaagattttgccacatttgattatatactatgtatggatgaaagcaatctgagagatttgaatagaaaaagtaatcaagttaaaacctgcaaagctaaaattgaactacttgggagctatgatccacaaaaacaacttattattgaagatccctattatgggaatgactctgactttgagacggtgtaccagcagtgtgtcaggtgctgcagagcgttcttggagaaggcccactgaggcaggttcgtgccctgctgcggccagcctgactagaccccaccctgaggtcctgcatttctcagtcggtg

SEQ ID NO: 618 - DNA, 240 bp, Homo sapiens
ctggcaagaagcatggatctcggaatccctgacctgctggacgcgtggctggagcccccagaggatatcttctcgacaggatccgtcctggagctgggactccactgcccccctccagaggttccgggccttcaagagagtgagcctgaagatttcttgaagcttttcattgatcccaatgaggtgtactgctcagaagcatctcctggcagtgacagtggcatctctgaggacccctgc

SEQ ID NO: 619 - DNA, 180 bp, Homo sapiens
gggcatggcgccacccgcggcgcctggccgggaccgtgtgggccgtgaggatgaggacggctgggagacgcgaggggaccgcaaggtgcaggccaagctggagaacgccgaagtgctggagctgacggtgcggcgggtccagggtgtgctgcggggccgggcgcgcgagcgcgagcagct

SEQ ID NO: 620 - DNA, 180 bp, Homo sapiens
ggttggagttgatgtgttggacagacatatagatccctctggaaagttgcacagccacagacttctcagcacagagtggggactgccttccattgtgaagtctatttcatttacaaacatggtttcagtagatgagagacttatatacaaaccacatcctcaggatccagaaaaaactgt

SEQ ID NO: 621 - DNA, 180 bp, Homo sapiens
ctaaaaaacacaaaggatgcagtacggaattctgtatgtcatactgcaaccgttatagcaaactcttttatgcactgtgggacaaccagtgaccagtttcttagagataatttggttctggtttcctctttcacacttcctgtcattggcttatacccctacctgtgtcattggccttaa

SEQ ID NO: 622 - DNA, 210 bp, Homo sapiens
gcgtttctcgccctgctgggatcgctgctcctctctggggtcctggcggccgaccgagaacgcagcatccacgagaatgccacgggtgacctggccaccagcaggaatgcagcggattcctctgtcccaagtgctcccagaaggcaggattctgaagaccactccagcgatatgttcaactatgaagaatactgcaccgccaacgcagtc

SEQ ID NO: 623 - DNA, 120 bp, Homo sapiens
agtcggatatcagatggcaagaaacaggagggaccagccactcaggttgacagtgctgtgggaacactccctgcaacaagtccccagagcacctccgtccaggccaaagggaccaacaag

SEQ ID NO: 624 - DNA, 225 bp, Homo sapiens
cgttctccagactttgccagctcctttaagattgtcctgtgacagcagccccagcgtgtgtcctggcaccctgtccaagaacctttctactgctggcccagcctggagctggcgctgtgcagcctcaccccgggcaggggcggccctcgttgtcagggcctctcctcactgctgttgtcattgctccgtttgtgtttgtactaatcagtaataaaggtttagaag

SEQ ID NO: 625 - DNA, 225 bp, Homo sapiens
aggaacagcttgaagtaccagagccctaccctccagcagaacccaggcccctagagtcctgctgtaggagtgagcctgagataccggagtcctctcgccaggaacagcttgaggaacagcttgaggtacctgagccctgccctccagcagaacccgggccccttcagcccagcacccaggggcagtctggacccccagggccctgccctagggtagagctggggg

SEQ ID NO: 626 - DNA, 225 bp, Homo sapiens
ccgtggaccaggagaaccaagatccaaggagatgggtgcagaaaccaccgctcaatattcaacgccccctcgttgattcagcaggccccaggccgaaagccaggcaccaggcagagacatcacaaagattgaggctccagggaccatagagtttgtggctgaccctgcagccctggccaccatcctgtcaggtgagggtgtgaagagctgtcacctggggcgcca

SEQ ID NO: 627 - DNA, 150 bp, Homo sapiens
aacgagaaggaatcctccagtctcggcaaatccaagaggaaataactggtaacacagaaacgtgatgcctttgacaccttgttcgaccatgccccagacaagctgaatgtggtgaaaaagacactcatcactttcgtgaacaagcacctg

SEQ ID NO: 628 - DNA, 300 bp, Homo sapiens
gctgctatggacgacattttcactcagtgccgggagggcaacgcagtcgccgttcgcctgtggctggacaacacggagaacgacctcaaccagggtatcgtcttggatgctttgtgaagagcaggtggaaaggaggcaattgcctagttcatcgtagaagtaatgatgtcttggactagaattaggggacgatcatggcttctcccccttgcactgggcctgccgagagggccgctctgctgtggttgagatgttgatcatgcggggggcacggatcaatgtaatgaaccgtggggatga

SEQ ID NO: 629 - DNA, 525 bp, Homo sapiens
cgagagcgggcagagaagatgggccagaatctcaaccgtattccatacaaggacacattctggaaggggaccacccgcactcggccccgtgagtcaccactgtgggaagaagggttgtaaaaggaaataatcctggcctcttggggctgggttagggtgaagctgggtacctgacctgcccacactcttaggaaatggaaccctgaacaaacactctggcattgacttcaaacagcttaacttcctgacgaagctcaacgagaatcactctggagaggtgacccctgcccttcttgcccttccctcactaaacccccataaattacttgctttgtacctgttttaagtttttcctccagttagtgggcaaggaagtggcagcaacatttcaagcctcctaacccctacctgtcctgcagctatggaagggccgctggcagggcaatgacattgtcgtgaaggtgctgaaggttcgagactggagtacaaggaagagcagggacttcaatgaagagtgtccccggc

SEQ ID NO: 630 - DNA, 300 bp, Homo sapiens
ccccaggctgatggggatgatgcccatgaagcccagctcctggtcatgcttcctgactcactgcactactcaggggtccgggccctggaccctgcggtgaggacctgggggcaggatggggtggggtcttgaggggctccagtaacccagactgaccttgccttctctcccattccaggagaagccactctgcctgtccaatgagaatgcctcccatgttgagtgtgagctggggaaccccatgaagagaggtgcccaggtcaccttctacctcatccttagcacctctgggatcagcat

SEQ ID NO: 631 - DNA, 225 bp, Homo sapiens
ctgaacgaggccaacgagtacactgcatccaaccagatggactatccatcccttgccttgcttggagagaaattggcagagaacaacatcaacctcatctttgcagtgacaaaaaaccattatatgctgtacaagagtatccggtctaaagtggagttgtcagtctgggatcagcctgaggatcttaatctcttctttactgctacctgccaagatggggtatcc

SEQ ID NO: 632 - DNA, 99 bp, Homo sapiens
caggcagaatattgtgaatgccaccgccaacctcggccagtccgtcaccctggtgtgcgatgccgaaggcttcccagagcccaccatgagctggacaaa

SEQ ID NO: 633 - DNA, 240 bp, Homo sapiens
ggtgaagttttggtaggtgagtgtcagagtgagccgacccaggccacatcctggcagtggaggcacagtcacccggggcagggccaggatcttggtatatcctcagatctcagtgggcagcgacatgaagtcaggcaatttcttgcaaccaccaccgaggccccgaaaagcactggtcgtcagggagctcctccccttggcccccagcctgtgccagccctggcccggctgccacacctc

SEQ ID NO: 634 - DNA, 250 bp, Homo sapiens
gatagcgtctggcgtccgcgcgctgcacaatggcggctctgaagagttggctgtcgcgcagcgtaacttcattcttcaggttcctgcttggctcgagtttgagtttacagcccctgcaagtaaatccaagagcctgttacagattggcggtcgtgccttatgaaatctgacttctacttccaggctgtttataccttaacttctctttaccgacaatatacaagtttacttgggaaaatgaattcagagg

SEQ ID NO: 635 - DNA, 450 bp, Homo sapiens
gaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaagagagaagcagcagccttgaaggaagtcctgatgaattttcaaatgactttggacaaagtttaccaaatgaaaagcaaacctcggggatactgtctgatcatcaacaatcacaattttgcaaaagcacgggagaaagtgcccaaa

SEQ ID NO: 636 - DNA, 650 bp, Homo sapiens
agtgcagacgcggctcctagcggatgggtgctattgtgaggcggttgtagaagttaataaaggtatccatggagaacactgaaaactcagtggattcaaaatccattaaaaatttggaaccaaagatcatacatggaagcgaatcaatggactctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggttattattcttggcgaaattcaaaggatggctcctggttcatccagtcgctttgtgccatgctgaaacagtatgccgacaagcttgaatttatgcaca

SEQ ID NO: 637 - DNA, 750 bp, Homo sapiens
atgtgcggccagcagaaggagtgtcctggctcctggcaacaggaccactgcccacctaagcttactgaggagccagtgctgatagcagtgcaacccctctttggcccacgggcaggaggcacctgtctcactcttgaaggccagagtctgtctgtaggcaccagccgggctgtgctggtcaatgggactgagtgtctgctagcacgggtcagtgaggggcagcttttatgtgccacaccccctggggccacggtggccagtgtcccccttagcctgcaggtggggggtgcccaggtacctggttcctggaccttccagtacagagaagaccctgtcgtgctaagcatcagccccaactgtggctacatcaactcccacatcaccatctgtggccagcatctaacttcagcatggcacttagtgctgtcattccatgacgggcttagggcagtggaaagcaggtgtgagaggcagcttccagagcagcagctgtgccgccttcctgaatatgtggtccgagacccccagggatgggtggcagggaatctgagtgcccgaggggatggagctgctggctttacactgcctggctttcgcttcctacccccaccccatccacccagtgccaacctagttccactgaagcctgaggagcatgccattaagtttgaggtctgcgtagatggtgaatgtcatatcctgggtagagtggtgcggccagggccagatggggtcccacagagcacgctc

SEQ ID NO: 638 - DNA, 150 bp, Homo sapiens
gccctatcccagtcccacttgtgtcaaaagcgaaatgggcccctggatggatagctactccggaccttacggggacatgcggcttccgcaacttacacgtggacgaccagatggctgtcattcagtactcctggatggggctcatggtgt

SEQ ID NO: 639 - DNA, 150 bp, Homo sapiens
gggcttctgcgaggcccccggcaacaggacccagagtggcaaccaccctgaggactggcctgtgtaccaggagctcctggggatggtcctgtccatctgcttgtgccggcacgtccattccgaagactacagcaaggtccccaagtactg

SEQ ID NO: 640 - DNA, 150 bp, Homo sapiens
tggggtcatccctatggccttctgcctcaactacgagatcaacgttcagtgctgcacccccactcgcggtaccacgaccgggtcatcttcagcccccacccccagcactgtgcagacgaccaccaccagtgcctggaccccaacgccgac

SEQ ID NO: 641 - DNA, 150 bp, Homo sapiens
ttggaaaactcgccaagggttatgtctggaatggaggaagcaacccacagctagtgccttagactctggaattcccttctaggcaaatcgacagacctccgacagcagttcagccaaaatgtctactccagcagacaaggtcttacggaa

SEQ ID NO: 642 - DNA, 150 bp, Homo sapiens
tgttgacaaagatactaccttgcctgcttcagctagaaaagttaagtcttcggaatcaaagattcgtgttcttctacaggaacgtggtgcccaggacagccggatccaggatctggaaactgagttggaaaagatggaagcaaggctaaa

SEQ ID NO: 643 - DNA, 150 bp, Homo sapiens
cgtgggaatccgccccactccgctccctgtgtccccaatggctctgcctacagtggggactatatggagcctgagaagccaggcgccccgcttctgcccccacctccccagaacagcgtcccccattatgccgaggctgacattgttacc

SEQ ID NO: 644 - DNA, 150 bp, Homo sapiens
tgccgcacagggtgtcccagagggatggtcaaggtcggtgattgtacaccctggagtgacatcgaatgtgtccacaaagaatcaggcatcatcataggagtcacagttgcagccgtagtcttgattgtggctgtgtttgtttgcaagtct

SEQ ID NO: 645 - DNA, 150 bp, Homo sapiens
aaccccaaaattcacctggcacagtcacttcacaagttgtctaccgcctgtccaggaaggacctatttttgaaggcataaaagcagttccatcaatggtgagcaccagcctgaatgcagaagcgctccagtatctccaagggtaccttca

SEQ ID NO: 646 - DNA, 200 bp, Homo sapiens
ttcacttcctgcacgaggagagcatcctggagcgggtgcagcagcacatcgagagcaagctcctgggctccaattcctccaggatgtacttcacccagaaagagacatcgggaagattctgatgtggaaatggtggaagatgattcccgaaaggaaatgactgcagcttgtaccccccggagaaggatcattaacctcac

SEQ ID NO: 647 - DNA, 150 bp, Homo sapiens
gcgcacggcgaggacgcgctgctggccgcccgggaggtgttcaagacccagggggtgatcaagtacatggggccggcaggtggaaaaccatgaattccttgtaaaaccttcatttgatcctaatctcagtgaattaagagaaataatgaa

SEQ ID NO: 648 - DNA, 710 bp, Homo sapiens
cctgaacctgaggagccccaacaacttcctgtcctactaccgcctcacacgcttcctctccagagtgatcaagtgtgacccagtaagtgagggtgatgtcccaggcagccttgccggggcttacagggggagacacctagtgccacggaaatgccgaggctggtgccaaggcccccaagggtgacaaggttggggctggggctgggcccctcggaccccaggccacagactgacagggcaccggcttcttccactgctcctagaacttactgactggctgggaggtcctcacagccttctcacgtcccctggggcttccaggagccgtagagtttctgggcgaagcgtccgggacggaggccccaggcggccccagccaatggtctgtgtggtgatggtgtgtggggttaggcccaggcgagctttgtttgggccacaatgtgcgtggccaataaatagatgcttgaaaagggctcctgtgaggtccgagacaccggacaacgggcggatagagacagccttgttgtttacggcctctttgagaggctgctgctgttaaaccctgggatgactgtgtctttcttcttaaaaatgccattgttttattcccgagtcttttcttaaagaaagaattaaaatgacaatcaaaagggtttgtggcatttaccaaattagaccagagaggtggccgggtcagccgccggccccgc

SEQ ID NO: 649 - DNA, 150 bp, Homo sapiens
tcagaagactcatctaactagacatatgcgtactcattcagtggggtatggataccatttggtaatatttactagagtgtgatctagatgggtgagaagccatttaaatgtgatcagtgcagttatgtggcctctaatcaacatgaagta

SEQ ID NO: 650 - DNA, 150 bp, Homo sapiens
ttgtactatcactggctggtctgagccctttccaccttaccctgtggcctgccctgtgcctctggagctgctggctgaggagggctgcccgtgctcttcactggcacgtgggtgagctgcaaactggccttcgaggacatcgcgtgctgg

SEQ ID NO: 651 - DNA, 150 bp, Homo sapiens
ccgaagggtccccgggacccgcctgctgagtggacccgggtgtaagtctaacgccagttcctgcacagagcagattcaagaaagaagatcaggaaggggcatgacccctgagttatgaaggggagaagggacagatgagcttccggagac

SEQ ID NO: 652 - DNA, 150 bp, Homo sapiens
aacgtgctgcgcgacatgggcctgcaggagatggccgggcagctgcaggcggccacgcaccagggcctgcactttatagaccagcaccgggctgcgcttatcgcgagggtcacaaacgttgagtggctgctggatgctctgtacgggaag

SEQ ID NO: 653 - DNA, 150 bp, Homo sapiens
gaagccatactgcggaggctggtggccctgctggaggaggaggcagaagtcattaaccagaaggagggcatcctggctgtttcacccgtggacttgaacttgccattggactgagctctttctcagaagctgctacaagatgacacctca

SEQ ID NO: 654 - DNA, 150 bp, Homo sapiens
tacagctttggaaaatgcatccatactcacctccagtttaacagcagaggacgatagaggttcagaagggttcttgaaaggccccctgtctgaagaaacagaagcatcggacagtgttgatggaggtcacgattctgtcattttggatcc

SEQ ID NO: 655 - DNA, 150 bp, Homo sapiens
atgggtaacaacttctccagtatcccctcgctgccccgaggaaacccgagccgcgcgccgcggggccacccccagaacctcaaagatagcgagctggtgctcccggactgtctgcggccgcgctccttcaccgccctgcggcggccgtcg

SEQ ID NO: 656 - DNA, 200 bp, Homo sapiens
cctgctggctgagggcttctacaccaccggcgcagtcaggcagatctttggcgactacaagaccaccatctgcggcaagggcctgagcgcaacgtttgtgggcatcacctatgccctgaccgttgtgtggctcctggtgtttgcctgctctgctgtgcctgtgtacatttacttcaacacctggaccacctgccagtcta

SEQ ID NO: 657 - DNA, 140 bp, Homo sapiens
atgtgcaataccaacatgtctgtacctactgatggtgctgtaaccacctcacagattcctgattgtaaaaaaactatagtgaatgattccagagagtcatgtgttgaggaaaatgatgataaaattacacaagcttcaca

SEQ ID NO: 658 - DNA, 695 bp, Homo sapiens
catttgaggaattccccatgaccccaacgacctacaaaggctctgtggacaaccagacagacagtgggatggtgctggcctcggaggagtttgagcagatagagagcaggcatagacaagaaagcggcttcaggtagctgaagcagagagagagaaggcagcatacgtcagcattttcttctctgcacttataagaaagatcaaagactttaagactttcgctatttcttctactgctatctactacaaacttcaaagaggaaccaggaggacaagaggagcatgaaagtggacaaggagtgtgaccactgaagcaccacagggaggggttaggcctccggatgactgcgggcaggcctggataatatccagcctcccacaagaagctggtggagcagagtgttccctgactcctccaaggaaagggagacgccctttcatggtctgctgagtaacaggtgccttcccagacactggcgttactgcttgaccaaagagccctcaagcggcccttatgccagcgtgacagagggctcacctcttgccttctaggtcacttctcacaatgtcccttcagcacctgaccctgtgcccgccgattattccttggtaatatgagtaatacatcaaagagtagtattaaaagctaattaatcatgtttataaaaaaaaaaaaaaaaaaaaaaaaaaaaaaa

SEQ ID NO: 659 - DNA, 180 bp, Homo sapiens
gtggtgccgcttgcagacattatcacgcccaaccagtttgaggccgagttactgagtggccggaagatccacagccagggcagcaactacctgattgtgctggggagtcagaggaggaggaatcccgctggctccgtggtgatggaacgcatccggatggacattcgcaaagtggacgcc

SEQ ID NO: 660 - DNA, 300 bp, Homo sapiens
gagcttggaaaaaagaagcttttgacctctttatggatcccagtttctttcagatggatgcctcttgtgttaatcagtaagttgccctcttatttgtattcagcatgatgcacctcacagtctgatgaaatcagccactcccctggaaagttagaatactgttctttaacagtaacaacataattacatgttgtaatccttatctctttcaggtggagagcaattatggacaatctgatgacacatgataaaacaacatttagagatttgatgactcgtgtagcagtggctcaaagcagt

SEQ ID NO: 661 - DNA, 180 bp, Homo sapiens
ccaacagaatacaggctggtgagattggagagatgaaggatggagtcccagagggagcacaacttcagggaccggttcatcgaaatccaacttaccgcccaagcaggggacctcctcgcccacgacctgccccagcagttggagaggctgaagataaagaaaatcagcaagccaccagtg

SEQ ID NO: 662 - DNA, 300 bp, Homo sapiens
caggaacaggtttaagtttttgaaactgaagtaggtctacacagtaggaactcatgtcatttcttgtaagtaaaccagagcgaatcaggcggtgggtctcggaaaagttcattgttgagggcttaagagatttggaactatttggagagcagcctccgggtgacactcggagaaaaaccaatgatgcgagctcagagtcaatagcatccttctctaaacaggaggtcatgagtagctttctgccagagggagggtgttacgagctgctcactgtgataggcaaaggatttgaggacctga

SEQ ID NO: 663 - DNA, 240 bp, Homo sapiens
gctgcgggcaggcgctggtgctcctgagctgctgcgtgcactgcttcagagtggagctcctgctgtgccccagctgttgcatatgcctgactttgagggactgtatccagtacacctggcggtccgagcctcaggtgcactgacctgctgcctgcccccagcccccttcccggaccccctgtacagcgtccccacctatttcaaatcttatttaacaccccacacccacccctcagttgg

SEQ ID NO: 664 - DNA, 180 bp, Homo sapiens
tcacagtactaaccgtcgtaggcggtctcgtagacgaaggactgatgaagatgctgttctgatggatggaatgactgaatctgatacagcttcagttaatgaaaatgggctaggcaaaagatgtgattgaagagcatggtccttcagaaaaggcaataaacggcccaactagtgcttctg

SEQ ID NO: 665 - DNA, 180 bp, Homo sapiens
gacagtgccacggtgtccggatatgatataatgaaatctaaaagcaaccctgacttcttgaagaaagacagatcctgtgtcacccggcaactcagaaacatcaggtccaagagtctgaaggaaggcctgacggtgcaagaacggttgaagctctttgaatccagggacttgaagaaagac

SEQ ID NO: 666 - DNA, 180 bp, Homo sapiens
atgtttcaacgtgcgcaagcgttgcggcggcgggcagaggactactacagatgcaaaatcaccccttctgcaagaaagcctctttgcaaccggcggatgataatctcaagacacctcccgagtgtctgctcactccccttccaccctcagctctaccctcagcggatgataatctcaaga

SEQ ID NO: 667 - DNA, 210 bp, Homo sapiens
atacaccctacaagtacaacctgaagaatttcatggttatcaactcagtggcctttgaccatgcagacccatccattttcacagtattgactgctttgagaaggccagcaaggtcaagctggcacctgagaggattgccgatggcaccatggcatttatgtttgaatcatctttaagtctggcggtcacaaagtggggactcaaggcctc

SEQ ID NO: 668 - DNA, 1150 bp, Homo sapiens
agccatgcagcccccgcccccgggcccgctgggcgactgcctgcgggactgggaggatctacagcaggacttccagaacatccaggagacccatcggctctaccgcctgaagctggaggagctgaccaaacttcagaacaattgcaccagctccatcacgcggcagaagaagcggctccaggagctggccctcgccctgaagaaatgcaaaccctccctcccagcagaggccgagggggccgcacaggagctggagaaccagatgaaagagcgccaaggcctcttctttgacatggaggcctatttgcctaagaagaatggattgtacctgagcctggttctggggaacgtcaacgtcacgctcctgagcaagcaggctaagtttgcctacaaggacgagtatgagaagttcaagctctacctcaccatcatcctcatcctcatctccttcacttgccgcttcctgctcaactccagggtgacagatgctgccttcaacttcctgctggtctggtactactgcaccctgaccatccgggagagcatcctcatcaacaacggctcccggatcaaaggctggtgggtgttccatcactacgtgtccaccttcctgtcgggagtcatgctgacgtggcccgacggtctcatgtaccagaaattccggaaccaattcctctccttttccatgtaccagagcttcgtgcagtttctccagtactactaccagagcggctgcctctaccgcctgcgggcgctgggcgagcggcacaccatggacctcactgtggagggcttccagtcctggatgtggcggggcctcaccttcctgctgccttttcttttctttggacacttctggcagctttttaacgcgctgacgttgttcaacctggcccaggaccctcagtgcaaggagtggcagggttgtgcaccacaagtttcacagtcagcggcacgggagcaagaaggattgaggctgggccttcccctgccggcccagaggggcttctgtcctgtgtgttgtgggaggggatgggaggcgcccctcgagtgtgcgtgtatcagggggtctcttctattctcccttgggttttatgggcgctgtgggccctgaaggaagacctgggcccagtgccctcaataaagagag

SEQ ID NO: 669 - DNA, 210 bp, Homo sapiens
gatcgcccgtggcaaaatcacagacctggccaacctcagtgcagccaaccatgatgctgccatctttccaggaggctttggagcggctaaaaacctcttgtgctgcattgcacctgtcctcgcggccaaggtgctcagaggcgtcgaggtgactgtgggccacgagcaggaggaaggtggcaagtggccttatgccgggaccgcagaggc

SEQ ID NO: 670 - DNA, 300 bp, Homo sapiens
ccctgcgtggctgggctgctcgggttagatcgtcaggtgagggaggaagggatagccagcgcgaaggaagtgctggagtcgtgtgttttggctgcgcgtgatcctgcgtgggtcgggaggtgtttctgtgtaggtgtctggccctttcatcagtcgtgcggaggaccgcgtgatttccttccagttctcctcggttttcaggtggtggcgccatcttcggaaaagcctaaagattagactgtaagaaaagaaaatagaagccatgtttcgaagacctgtattacaggtacttcgtcagtt

SEQ ID NO: 671 - DNA, 180 bp, Homo sapiens
tgtttggcatgcggaacagtgcagccagtgatgaggactcaagctgggctaccttatcccagggcagcccctcctatggctccccagaggacacagcctcccacctggcagattccttctggaaccccaacgccttcgagacggattccgacctgccggctggatggatgagggtccagg

SEQ ID NO: 672 - DNA, 180 bp, Homo sapiens
agttgggaaatactacagtaatctgtggagttaaagcagaatttgcagcaccatcaacagatgcccctgataaaggatacgttgattccggtctggacctcctggagaagaggcccaagtggctagccaattcattgcagatgtcattgaaaattcacagataattcagaaagaggactt

SEQ ID NO: 673 - DNA, 360 bp, Homo sapiens
  misc_feature (26)..(26): n is a, c, g, or t
ccaggagctcagaccgtctttgagantctcccgaaggaggaatgggagggtaggggcgctgccagactccttccctggtgggcctagatgaagacgctcaaggaccctcgtgacttggccgagacaggggaagggagaagttgagtcgggcaaggaagagatgctaaagcctggggaattaagaacatgccagaatcatcccgagggagtctggaattagggagggtgaggactcgctaggatcgtcctgtggatctggctacagcaggagctgatgaccctcatgatgtctggcgataaagggatttctgccttccctgaatcagacaaccttttcaaatgggtagggaccatccatgg

SEQ ID NO: 674 - DNA, 180 bp, Homo sapiens
atttcagagtgcctgccccggttgacatgcatgatcagagggatcggagacccactagtgtcggtgtatgcccgtgcctacctgtgccgggctctgctgaccgagatgatggaaaggtgtaagaaactaggaaacaatgccttgctgttgaattctgtgatgtctgccttccgggctgag

SEQ ID NO: 675 - DNA, 180 bp, Homo sapiens
agcacagaaggaagaagttcttagccacatgaatgatgtgctagagaatgagctccaatgtattatttgttcagaatacttcattgagcaaagagattgttctgaagaccgtgctctaagggcatttgaaagactgccaggtagtgcgagcctgagatggtctggaggattctctctagc

SEQ ID NO: 676 - DNA, 180 bp, Homo sapiens
ggggctgcaggggaggccgcggcggggaaaatggcggacgggaaggcgggagacgagaagcctgaaaagtcgcagcgagctggagccgccggagatacaccaacatcagctggaccaaactccttcaataaaggaaagcatgggttttctgataaccagaagctgtgggagcgaaatata

SEQ ID NO: 677 - DNA, 240 bp, Homo sapiens
tgcgttttgagtctcgggacccctgttggagagactatggcgctcaacaagaatcactcggagggcggcggagtgatcgtcaataacaccgagaggtgaaaacactgcggaaggatcctggaggaccaaagttcgggtgtcgaggaagtgggcgcatcctaatgtcctatgatcacgtggaactcacattcaatgacatgaagaacgtgccagaagccttcaaagggaccaagaaaggca

SEQ ID NO: 678 - DNA, 180 bp, Homo sapiens
acaattgccacgggtactggcaattggttttcggctttggcgctcggggtgactcttctcaaatgccttctcatccccacatagcaacttcagagtggacgttggattacccccctttctttgcatggtttgagtatatcctgtcacatgttgccaaatattttgatcaagaaatgctga

SEQ ID NO: 679 - DNA, 210 bp, Homo sapiens
tccggttcgtgttcgtccgcggagatctctctcatctcgctcggctgcgggaaatcgggctgaagcgactgagtccgcgatggagaaaactttagaaactgttcctttggagaggaaaaagagagaaaaggaacagttccgtaagctctttattggtggcttaagctttgaaaccacagaagaaagtttgaggaactactacgaacaatg

SEQ ID NO: 680 - DNA, 350 bp, Homo sapiens
aggcgcaagccggcaagatggcggcggctggggctggccgtctgaggcgggtggcatcggctctgctgctgcggagcccccgcctgcccgcccgggagctgtcggccccggcccgactctatcacaagaaggtatctcaaatctgtgaagtattgtagaggagacacaaaaggaattgggggtcacaaatggttctcattgacatgagtgtagacctttctactcaggttgttgatcattatgaaaatcctagaaacgtggggtcccttgacaagacatctaaaaatgttggaactggactggtgggggctccagcatgtggtgacgtaatgaaattacagattcaagtg

SEQ ID NO: 681 - DNA, 438 bp, Homo sapiens
cacagccttgtagccgggagtcgctgccgagtgggcgctcagttttcgggtcgtcatggctggctacgaatacgtgagcccggagcagctggctggctttgataagtacaagcccccgaaaggatggagttccttctgttgtgtcaatcgccttcattttagtgaagtttccactcgcctgtcatgcatacaacttcggaggaggagatgatcgtttggcagatgaggcccgggaggggagcgacttgccgatgccatcctgctgatgtctccacttctgctcccggcagggacttcctaagcggcagcttgtggcgctagggccaccagatgaaagggaggtgcacaggaaggagctgtggagtggaaagagcgcgggctttcgagcacatacaaacctgattacaaaagtcagatttctttaaaaaaaaaaaaaaa

SEQ ID NO: 682 - DNA, 280 bp, Homo sapiens
aggagcacagtgcggccatttcctgggccacatgacagggcacccctgccccgtccccacctcgggacaccatgggccacgcccatgttttccaggcccccagcctcccactcgactttcctcttaggaacctggcccctccctggcactgaggccctgacccctgctcccggccacaggcagtggagaaagccaggtggccacgtttttcagcttcgcatccatgataagctgaaagcgctttcttgctcccgcccactcctctgctctgcctagttga

SEQ ID NO: 683 - DNA, 150 bp, Homo sapiens
gatgtagaattttgcctgagtttgacccaatatgaatctggttccatggataaagctgccaatttcagctttagaaatacactggaagtatttttgagcagtggctccgaaggcaccgtcctcttcaagaagtttatccagaagccaatg

SEQ ID NO: 684 - DNA, 210 bp, Homo sapiens
aggaacagatgcaggaatggacttggctctgtaaaggatggggaacctcacttcgtggtggtccactgcacaggctacatcaaggcctggcccccagcaggtgtttccctcccagatgatgacccagcctgaggtcttccaggagatgctgtccatgctgggagatcagagcaacagctacaacaatgaagaattccctgatctaactat

SEQ ID NO: 685 - DNA, 350 bp, Homo sapiens
atgaaaggaaaaagaggcgacgagaaagaaataagattgcagctgcaaagtgccgaaacaagaagaaggagaagacggagtgcctgcagcttcagtattagcagagccacaggccgcctctgtggcatcaccagggtttctctgaagaagagggtctgcattttcctaaacccagtgctgctctcccatctcccatcttcctctcgcagcttgatgagccccggtgtgtcccaggagtcggagaagctggaaagtgtgaatgctgaactgaaggctcagattgaggagctcaagaacgagaagcagcatttgatatacatgctcaaccttcatcggcccacgtgtattgt

SEQ ID NO: 686 - DNA, 280 bp, Homo sapiens
accccccgcagcagcagcagcagcagcagcaacgacatgattcctatggcaatcagttctccacccaaggcaccccttctggcagccccttccccagccagcagactacaatgtatcaacagcaacagcaggaaccccggaggcatggcgggtaatgatgtccctcaagtctggtctcctggcagagagcacatgggcattagataccatcaacatcctgctgtatgatgacaacagcatcatgaccttcaacctcagtcagctcccagggttgctagag

SEQ ID NO: 687 - DNA, 280 bp, Homo sapiens
accccccgcagcagcagcagcagcagcagcaacgacatgattcctatggcaatcagttctccacccaaggcaccccttctggcagccccttccccagccagcagactacaatgtatcaacagcaacagcaggtatccagccctgctcccctgccccggccaatggagaaccgcacctctcctagcaagtctccattcctgcactctgggatgaaaatgcagaaggcaggtcccccagtacctgcctcgcacatagcacctgcccctgtgcagccccccat

SEQ ID NO: 688 - DNA, 210 bp, Homo sapiens
gaggctcacggaatttgaagacacccccaccagtcagttgaccattgatgagttcatgaagatcgacctggaggaggagtgcgacccccccatcgaggagggagggcagacggaggcccgagagcctccccaggcctcttcgtgggaaggccccagtaccactcgtaggaggtctcagctctggcatggctgccccggatgtggccgagg

SEQ ID NO: 689 - DNA, 980 bp, Homo sapiens
cggccgcgtcgaccggctgcgctcaccggtaggccccgctcgggttccgccgaagcccagcccccgcaggtcggcccctccgacgccggccgcgccgcaagggaggccagctcgctcgcagtggggaggtcgcggctccagtcctcgcgtccccgccgtggtcccggtgcctgtcccatcccgcgggcggggccgttgcggggccgggcccgggccggggcgaatctgcggctgcgaatcggctggagcggggcctcgcgagaggccgaggctgggcggctgggctgggcgggcggccggggctgctccggaggctcgggtggcttgagagtcttgggaggctccgcctgcccgccggtcgccggcatgacgggccgcgtgtgccgcggttgcggcggcacggacatcgagctggacgcggcccgcggggacgcggtgtgcaccgcctgcggctcagtgctggaggacaacatcatcgtgtccgaggtgcagttcgtggagagcagcggcggcggctcctcggccgtgggccagttcgtgtccctggacggtgctggcaaaaccccgactctgggtggcggcttccacgtgaatctggggaaggagtcgagagcgcagaccctgcaggatgggaggcgccacatccaccacctggggaaccagctgcagctgaaccagcactgcctggacaccgccttcaacttcttcaagatggccgtgagcaggcacctgacccgcggccggaagatggcccacgtgattgctgcctgcctctacctggtctgccgtacggagggcacgccgcacatgctcctggtcctcagcgacctgctccaggtgaatgtgtacgtgcttggaaagacgtttcttctcttggcaagagagctctgcatcaatgcgccggccatagacccgtgcctgtatattccacgctttgcgcacctgctggaattcggggagaagaaccacgaggtgtccat

SEQ ID NO: 690 - DNA, 210 bp, Homo sapiens
ctccgccactccggtaggattccccgcctgtcattccctagcccagctcttgggaaactgcagaggggtccagaggatttgcagttctgaacctgcacactccagtctaggatctccgagcaagagcgtagcctcatggctacaacctgtgagattagcaacatttttagcaactacttcagtgcgatgtacagctcggaggactccacc

SEQ ID NO: 691 - DNA, 212 bp, Homo sapiens
gctgcgagacctcacttccagctcttctgatgagctcagttggatcattgagctgctggagaaggatggcatggccttccaggaggccctagacccagggccctttgaccagggcagcccctttgcccaggagctgctggacgacgtctccaccgcagggactggtgcttctcggagctcccactcctcagactccggtggaagtgacgtgg

SEQ ID NO: 692 - DNA, 210 bp, Homo sapiens
ccgcaaggcccggaagcccctggtggagaagaagcggcgcgcgcggatcaacgagagcctgcaggagctgcggctgctgctggcgggcgccgaggccaagctggagaacgccgaagtgctggagctgacggtgcggcgggtccagggtgtgctgcggggccgggcgcgcgagcgcgagcagctgcaggcggaagcgagcgagcgcttcgc

SEQ ID NO: 693 - DNA, 420 bp, Homo sapiens
ctgctgctggcgggcgccgaggtgcaggccaagctggagaacgccgaagtgctggagctgacggtgcggcgggtccagggtgtgctgcggggccgggcgcgcggtgagtggcggcggggcgggcgggggcgccggccgcgggcgcctgtaacccctgccagacggaggacttccctcccggcgcccctgtcctgtcggcggcgagggctcccaccggagcagggtgcgcccccgcgtctcctgggtgagccgcgtccccgcgggccgggtgggctgggccacgcagtcgccgctcaccgcgcgggacgcggctctctccctcccaccctcgggcccagagcgcgagcagctgcaggcggaagcgagcgagcgcttcgctgccggctacatccagtgcatgcacgaggtgcacacgttcgt

SEQ ID NO: 694 - DNA, 210 bp, Homo sapiens
gaagcgccgacgagaccggatcaataacagtttgtctgagctgagaaggctggtacccagtgcttttgagaagcaggtaatggagcaaggatctgctaagctagaaaaagccgagatcctgcagatgaccgtggatcacctgaaaatgctgcatacggcaggagggaaaggttactttgacgcgcacgcccttgctatggactatcggag

SEQ ID NO: 695 - DNA, 350 bp, Homo sapiens
caccacccccagccggctacctaccagacttccgggaacctgggggtgtcctactcccactcaagttgtggtccaagctatggctcacagaacttcagtgcgccttacagcccctacgcgttaaatcaggaagcagacccaccaagaagcctgtcgctcccccgcatcggagacatcttctccagcgcagacttttgactggatgaaagtcaaaagaaaccctcccaaaacagggaaagttggagagtacggctacctgggtcaacccaacgcggtgcgcaccaacttcactaccaagcagctcacggaactggagaaggagttccacttcaacaagtacctgacgcgcg

SEQ ID NO: 696 - DNA, 210 bp, Homo sapiens
agcctgtcgctcccccgcatcggagacatcttctccagcgcagacttttgactggatgaaagtcaaaagaaaccctcccaaaacagggaaagttggagagtacggctacctgggtcaacccaacgcggtgcgcaccaacttcactaccaagcagctcacggaactggagaaggagttccacttcaacaagtacctgacgcgcgcccgcag

SEQ ID NO: 697 - DNA, 210 bp, Homo sapiens
cgtgaagaactccaaaaataaaattctctagagataaaaaaaaaaaaaaaaggaaaatgccagctgatataatggagaaaaattcctcgtccccggtagcagccagtgtcaacacgacaccggataaaccaaagacagcatctgagcacagaaagtcatcaaagcctattatggagaaaagacgaagagcaagaataaatgaaagtctga

SEQ ID NO: 698 - DNA, 210 bp, Homo sapiens
acatctccgcggagcagaagcggcgcttcaacatcaagctggggtttgacacccttcatgggctcgtgagcacactcagtgcccagcccagcctcaaggagcgtgcgggcttgcaggaggaggcccagcagctgcgggatgagattgaggagctcaatgccgccattaacctgtgccagcagcagctgcccgccacaggggtacccatca

SEQ ID NO: 699 - DNA, 280 bp, Homo sapiens
ggcccggcagggggttccaaggaaatggggaccagcagcctgggcctggtggacaccacaggaggcccaggcgatgactacggggtgcttgggagcactgccaatgagacagagaagaaatcatccaggcggagaaaggagagttcaggtcaaagtgtggttccagaaccgaaggatgaagtggaagcgtgtgaagggaggtcagcccatctcccccaatgggcaggaccctgaggatggggactccacagcctctccaagttcagagtgagattctgca

SEQ ID NO: 700 - DNA, 210 bp, Homo sapiens
tgtgaaggtgcatggaggaagaaaggagaaaacagagatcctatcagatgaccttacagacaaagcagagtattctgccagtcactcccaaattgtttcagtttaaaaggatcatgaattttctaaaactgaggaactaaaactagaagatgtggatgaggaaattaatgctgaaaatgtggaaagcaagaagaaaactgtgggagatga

SEQ ID NO: 701 - DNA, 700 bp, Homo sapiens
gagcagacgcctccaggatctgtcggcagctgctgttctgagggagagcagagaccatgtctgacatagaagaggtggtggaagagtacgaggaggagtgaagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagacagagcggaaaagtgggaagaggcagactgagcgggaaaagaagaagaagattctggctgagaggaggaaggtgctggccattgaccacctgaat

SEQ ID NO: 702 - DNA, 210 bp, Homo sapiens
gaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgctttgaggggtctc

SEQ ID NO: 703 - DNA, 210 bp, Homo sapiens
ctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcaggatgtgcgacgtgtg

SEQ ID NO: 704 - DNA, 1082 bp, Homo sapiens
ctttgccagtccatcttcaaattggaattatagaaagtagagggagggatagtctaccgtctctcactggattggtgccacctaaaacattgttatgctggaaatgctagaatataatcactatcaggtgcagacccacctcgaaaaccccaccaagtaccacatacagcaagcccaacggcagcaggtaaagcagtacctttctaccactttagcaaataaacatgccaaccaagtcctgagcttgccatgtccaaaccagcctggcgatcatgtcatgccaccggtgccggggagcagcgcacccaacagccccatggctatgcttacgcttaactccaactgtgaaaaagagtttatgaagcagtgagaatgcagagagaggagaaggggaggtggaaaaggaaaagcaaaaatagaagaggtgtgggacatgctgtttagaagttccgcttgttgtgaatgtctggaatattatttttatttctccctgagttgggggaagaaagaatggaatatgcatggatggatttgaatcatatagcacatgagactttaacggaaacgcaaaggtttaattgctggatacattctgtttcataataaaattgccactgcccgttaaatctgctttggtgaaggctggattggaaacaagactcaaactaccttcaagctaattggtgcatcaaaatttgcagcatacaaatacctgagagctgtgatttaatgctcattatttccaaattatgagatgatgagcttcatctcaatgggatttaccgtactatggactatgaagtgtttatgcaaattcggaggcaacttttctagagttggattgattttaatttctagagggactaaaatctttgcccctatgcccaaaccaactgctttatttttctctacccaaatttgtcatctagcaagatgatttgacacaagttcttccttcattatttcatcttttggtcagattccactttgtttgaaagcttagttcatcttgttgctgtgccatcagctttgtgtgaacaggtcattaaaaagtcatttgcaaatccaaaaaaaaaaaaaaa

SEQ ID NO: 705 - DNA, 1623 bp, Homo sapiens
agagtccctgtgagacggtttcacagaaggatgtgtatttacccaaagctacacatcaaaaagaattcgataccttaagtggaaaattagaagcctacctgtggaaggaaagtttctcttccaaataaagccttagaattaaaggacagagaaacattcaaagcagagtctcctgataaagatggtcttctgaagcctacctgtggaaggaaagtttctcttccaaataaagccttagaattaaaggacagagaaacactcaaagcagagtctcctgataatgatggtcttctgaagcctacctgtggaaggaaagtttctcttccaaataaagctttagaattgaaggacagagaaacattcaaagcagctcagatgttcccatcagaatccaaacaaaaggatgatgaagaaaattcttgggattttgagagtttccttgaggctctcttacagaatgatgggtgtttacccaaggctacacatcaaaaagaattcgataccttaagtggaaaattagaagagtctcctgataaagatggtcttctgaagcctacctgtggaaggaaagtttctcttccaaataaagccttagaattaaaggacagagaaacactcaaagcagagtctcctgataaagatggtcttctgaagcctacctgtgtaaggaaagtttctcttccaaataaagccttagaattaaaggacagagaaacattaaaagcagctcagatgttcccatcagaatccaaacaaaaggatgatgaagaaaattcttgggattttgagagtttccttgagactctcttacagaatgatgtgtgtttacccaaggctacacatcaaaaagaattcgataccttaagtggaaaattagaagatttcaggccgggcactgtggttcacgcctgtaatcccagccctttgggaggcagaggcatgcggatcacgaggtcagcagatcgagaccatcctggctaacatggtgaaaccccgtctctatgaaaaaatacaaaaaattagccaagcatggtggtgggtgcctctagtcccagctactcgggaggctgaggcaggagaatgtgagaacccatgaggcagagattgcagtgagccaagatcatgcacctacactccagcctgggtgacagggccagactctgtgaaaaaaaaaaaaaaaaaagaatttatttattgtggcactattcacaacagcaaagacttggaaccaaaccaaatgtccaacaacgctagactggattaagaaagtatggcacatatacaccatggaacactacgcagccataaaaaatgataagttcatgtcctttgtagggacatgaatgaaactggaaaccatcattctcagcaaactctcgcaaggacaaaaaaccaaacactgcgtgttctcactcataggtgtgaattgaacaatgagaacacatggacacaggaaggggaacatcacactccggggactgttgtggggttggaggagggatagcattaggagatatacctaatgctaaatgacgagttaatgggaacctgcacattgtgcacatgtaccctaaaacttaaagtataatattaaaataaaaaataaagaaaaaaaaaaaaaaa

SEQ ID NO: 706 - DNA, 700 bp, Homo sapiens
aaaaatggcggacggaggagcagcgagtcaagatgagagttcagccgcggcggcagcagcagcagctactactgggctgtaaacagtgatgccagcaaaatgttacttcagctgatgaagtgatgctgtttcgagaatttgaaagcaatttttcagtggataaagaagttgacagcacgatttgttggatgtgatgaaggattaatcagcatacaccttcacttgtattagcttaagatggaatggttctgggcaatataaaataacagactcaagaatgaacaatccgtcagaaaccagtaaaccatctatggagagtggagatggcaacacagcatggacccttttatgatatgggcactgaaactaaagcacatggtggaagaaggattggtagcatatagaaacatttttagacaaatgaaaaagcaaaaaagtcagaaattacagtgtatttccataaagttacaccaagtgtgcctgcctctcctgcctccccttccagctttttgtcttctgccatttctgagtcagcaagacccctcctgttcctccttctcagcctactcagcatgaagacaaggatgaagatctttgtgatgatccacttccacttaatgaatagcacacaaaccaatggtctggactttcagaagcagcctgtgcctgtaggaggagcaatctcaacagcccaggcgca

SEQ ID NO: 707 - DNA, 140 bp, Homo sapiens
gaggagcagcgagtcaagatgagagttcagccgcggcggcagcagcagcagactcaagaatgaacaatccgtcagaaaccagtaaaccatctatggagagtggagatggcaacacaggcacacaaaccaatggtctggac

SEQ ID NO: 708 - DNA, 210 bp, Homo sapiens
gctacagcccccatatggtcacaccccaagggggcgcggggaccttaccgttgtcccaagcttccagcagtctgagcacaacagcacaaaccccagccctcaaggcagccactcggctatcggcttgtcaggcctgaaccccagcacgggccctggcctctggtggaaccctgccccttaccagccttgatggcagcgggaatctggtgc

SEQ ID NO: 709 - DNA, 700 bp, Homo sapiens
acggcctcccctcctgtttccagcgcctccaatgacccagtgggatcctactccatcaatgggatcctggggattcctcgctccaatggtgagaagaggaaacgtgatgaagttgaggtatacactgatcctgcccacattagaggaggtggaggtttgcatctggtctggactttaagagatgtgtctgagggctcagtccccaatggagattcccagagtggtgtggacagtttgcggaagcacttgcgagctgacaccttcacccagcagcagctggaagctttggatcgggtctttgagcgtccttcctaccctgacgtcttccaggcatcagagcacatcaaatcagaacaggggaacgagtactccctcccagccctgacccctgggcttgatgaagtcaagtcgagtctatctgcatccaccaaccctgagctgggcagcaacgtgtcaggcacacagacatacccagttgtgactggtcgtgacatggcgagcaccactctgcctggttacccccctcacgtgccccccactggccagggaagctaccccacctccaccctggcaggaatggtgcctgggagcgagttctccggcaacccgtacagccacccccagtacacggcctacaacgaggcttggagattcagcaaccccgccttactaagttccccttattattatagtgccgccc

SEQ ID NO: 710 - DNA, 225 bp, Homo sapiens
cgcccccgcagctgccgccgccgccagggcccggactcggacgcgtggtagcctagagtcctggggagcttctgtccacctgtcctgcagaggagtcgtttccagcccgggccccaggatgggtgagttcaacgagaagaagacaacatgtggcaccgtttgcctcaagtacctgctgtttacctacaattgctgcttctggctggctggcctggctgtcatggc

SEQ ID NO: 711 - DNA, 1834 bp, Homo sapiens
cccgccttccatacctccccggctccgctcggttcctggccaccccgcagcccctgcccaggtgccatggccgcattgtaccgccctggcctgcggcttaactggcatgggctgagccccttgggctggccatcatgccgtagcatccagaccctgcgagtgcttagtggagatctgggccagcttcccactggcattcgagattttgtagagcacagtgcccgcctgtgccaaccagagggcatccacatctgtgatggaactgaggctgagaatactgccacactgaccctgctggagcagcagggcctcatccgaaagctccccaagtacaataactgctggctggcccgcacagaccccaaggatgtggcacgagtagagagcaagacggtgattgtaactccttctcagcgggacacggtaccactcccgcctggtggggcctgtgggcagctgggcaactggatgtccccagctgatttccagcgagctgtggatgagaggtttccaggctgcatgcagggccgcaccatgtatgtgcttccattcagcatgggtcctgtgggctccccgctgtcccgcatcggggtgcagctcactgactcagcctatgtggtggcaagcatgcgtattatgacccgactggggacacctgtgcttcaggccctgggagatggtgactttgtcaagtgtctgcactccgtgggccagcccctgacaggacaaggggagccagtgagccagtggccgtgcaacccagagaaaaccctgattggccacgtgcccgaccagcgggagatcatctccttcggcagcggctatggtggcaactccctgctgggcaagaagtgctttgccctacgcatcgcctctcggctggcccgggatgagggctggctggcagagcacatgctgatcctgggcatcaccagccctgcagggaagaaggcgctatgtgcagccgccttccctagtgcctgtggcaagaccaacctggctatgatgcggcctgcactgccaggctggaaagtggagtgtgtgggggatgatattgcttggatgaggtttgacagtgaaggtcgactccgggccatcaaccctgagaacggcttctttggggttgcccctggtacctctgccaccaccaatcccaacgccatggctacaatccagagtaacactatttttaccaatgtggctgagaccagtgatggtggcgtgtactgggagggcattgaccagcctcttccacctggtgttactgtgacctcctggctgggcaaaccctggaaacctggtgacaaggagccctgtgcacatcccaactctcgattttgtgccccggctcgccagtgccccatcatggacccagcctgggaggccccagagggtgtccccattgacgccatcatctttggtggccgcagacccaaaggggtacccctggtatacgaggccttcaactggcgtcatggggtgtttgtgggcagagccatgcgctctgagtccactgctgcagcagaacacaaaaggacttctgggaacaggaggttcgtgacattcggagctacctgacagagcaggtcaaccaggatctgcccaaagaggtgttggctgagcttgaggccctggagagacgtgtgcacaaaatgtgacctgaggcctagtctagcaagaggacatagcaccctcatctgggaatagggaaggcaccttgcagaaaatatgagcaattgatattaactaacatcttcaatgtgccatagaccttcccacaaagactgtccaataataagagatgcttatctattttaaaaaaaaaaaaaaaaaa

SEQ ID NO: 712 - DNA, 140 bp, Homo sapiens
ttagacagcgcagggccatggctgaggcggccccggccccgacatctgaatgggactccgagtgccttacatccctgcagccccttcctcttcctacacccccagcagcaaatgaggcacacctgcagacagcagctatc

SEQ ID NO: 713 - DNA, 210 bp, Homo sapiens
ctgccgccacccccgagatcagagtcaaccacgagccagagccggccggcggggccacgcccggggccaccctccccaagtccccatctcagcccacagagagtccagccggcagcctgccttccggggagcccagcgctgccgagggcacctttgctgtgtcctggcccagccagacggccgagccggggcctgcccaaccagcagagg

SEQ ID NO: 714 - DNA, 210 bp, Homo sapiens
ctgccgccacccccgagatcagagtcaaccacgagccagagccggccggcggggccacgcccggggccaccctccccaagtccccatctcagtttgaggccccggggcctttctcggagcaggccagtctgctggacctggactttgaccccctcccgcccgtgacgagccctgtgaaggcacccacgccctctggtcagtcaattccat

SEQ ID NO: 715 - DNA, 150 bp, Homo sapiens
cgccatctttatagcccaaatgaatggtgttgtcctggatggaggacagattgtgactgtaagggacaggatgagaacttcagtcaatgttgtgggtgactcttttggggctgggatagtctatcacctctccaagtctgagctggatac

SEQ ID NO: 716 - DNA, 150 bp, Homo sapiens
gatgggagatcaggccaagctgatggtggatttcttcaacattttgaatgagattgtaatgaagttagtgatcatgatcatgtgtgctggaactttgcctgtcacctttcgttgcctggaagaaaatctggggattgataagcgtgtgac

SEQ ID NO: 717 - DNA, 150 bp, Homo sapiens
agactaagatggttatcaagaagggcctggagttcaaggatgggatgaacgtcttaggtctgatagggtttttcattgcttttgtgctggaactttgcctgtcacctttcgttgcctggaagaaaatctggggattgataagcgtgtgac

SEQ ID NO: 718 - DNA, 280 bp, Homo sapiens
gaagagcccaatgacatgattactgagagttcactggatgttgctgaagaagaaatcatagacgatgatgatgatgacatcacccttacagtggaaacagggtttctccatgttggccagtctcagactcctgacctcaagcaatctgcttgcctcggcttcccaaagtgcgggattacaggaatgagccactgcgccagccaggtttgttgaagcttcttgtcatgacggggatgaaacaattgaaactattgaggctgctgaggcactcctcaatatg

SEQ ID NO: 719 - DNA, 280 bp, Homo sapiens
gagcagcggcggcggcggcggcggcggcagcagcagcttcagtagcgcagaggcggcggtggcgagaggtgcggcgaaggaggcagaggcacttatgcttgtcaggccaagaagcttgagagaagaaaaatttcagaaaaattgtctcaatttgactagaatatcaatgaaccaggaaaaaaggaagaaaaactaaaccaccatgaccagattccccagccactacgccaaatatatctgtgaagaagaaaaacaaagatggaaagggaaacacaattta

SEQ ID NO: 720 - DNA, 840 bp, Homo sapiens
ggattggtaccgtaaccatggtcagctggggtcgtttcatctgcctggtcgtggtcaccatggcaaccttgtccctggcccggccctccttcagtttagttgaggataccacattagagccagaaggagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcct

SEQ ID NO: 721 - DNA, 210 bp, Homo sapiens
tgtcttctctgctctggtggagtatggcaccttgcattattttgtcagcaaccggaaaccaagcaaggacaaagataaaaagaagaaaaaccctgcccctaccattgatatccgcccaagatcagcaaccattcaaatgaataatgctacacaccttcaagagagagatgaagagtacggctatgagtgtctggacggcaaggactgtgc

SEQ ID NO: 722 - DNA, 210 bp, Homo sapiens
tgtcagtaaacgggcaggtactcagtgcaccaactgccagacgaccaccacgacactgtggcggagaaatgccagtggggatcccgtgtgcaatgcctgcggcctctactacaagctacaccaccagcactactgtggtggctccgctcagctcatgagggcacagagcatggcctccagaggaggggtggtgtccttctcctcttgtag

SEQ ID NO: 723 - DNA, 210 bp, Homo sapiens
agtgagtcggccgtcagcagcaccgtcaaccctgtcgccattcacaagcgcagcaaggtcaagaccgagcctgagggcctgcggccggcctcccctctggcgctgacgcaggagcagctggctgacctcaaggaagatctggacagggatgactgtaagcaggaggctgaggtggtcatctatgagaccaactgccactgggaagactgc

SEQ ID NO: 724 - DNA, 350 bp, Homo sapiens
cggctttctgcaaagaccatgactccaggtctggaaaacaaccttcacagaccctatctccttcagatttcttggacaagttaatgggaaggacatcaggatatgatgcaagaatcaggccaaattttaaagggcctcctgtaaatgttacctgcaacatatttatcaacagctttgggtcaatagcagaaactacaatggactaccgagtgaatatttttctgagacaacagtggaatgattcacggctggcgtacagtgagtacccagatgactccctggacttggacccatccatgctagactccatttggaaaccagatttgttctttgccaatgagaagggtgcc

SEQ ID NO: 725 - DNA, 140 bp, Homo sapiens
gcttgagcaacaagaaaatctaccaggaggaggagaaggagaaacgtggccgcaggaaggcgagcgagctgcgcatccacgacctggaggacgacctggagatgtcgtccgatgccagtgatgccagtggtgaggagggg

SEQ ID NO: 726 - DNA, 280 bp, Homo sapiens
cccgcaggagaagaagcgcaggaaagacagcagcgaggagtcggacagctcagaggagagcgacattgacagcgaggcctcctcagccctcttcatggcggtaaggcccagcccggtggcgggggaggcctgggcgtctgtttgcagactcacccagctcccagccctgacctctgcagaagaagaagacgccacccaagagagagcggaagccgtcgggagggagctcaaggggcaacagccgcccaggcacgcccagcgcagagggtggcagcacctc

SEQ ID NO: 727 - DNA, 200 bp, Homo sapiens
gggcggctccaggagctcacccccagttcaggtgaccctggagagcatgacccagcgtccacacacaaatccacacgccctgtgaagaaggtctccacccctgtccctgccttacccagcaagcttcccacgtttggagccccggaacagttagtggatttaaaacaagctggcttggaggctgcagccaaagccaccag

SEQ ID NO: 728 - DNA, 280 bp, Homo sapiens
aaaactttctgtgtgaatggaggggagtgcttcatggtgaaagacctttcaaacccctcgagatacttgtgcaagtgccaacctaacttcactggagacagatgtactgagaatgtgcccatgaaagtccaaaaccaagaaaaggcggaggagctgtaccagaagagagtgctgaccataaccggcatctgcatcgccctccttgtggtcggcatcatgtgtgtggtggcctactgcaaaaccaagaaacagcggaaaaagctgcatgaccgtcttcggc

SEQ ID NO: 729 - DNA, 210 bp, Homo sapiens
ggggacaaccctcccgtcctgttcagcagcgacttccgcatctctggggcaccagagaagtacgagtccaaagaggtttctaccctggaatctcactgagtgccccaggagagcgagaggcgcctgggatctgagaggaggctgctgggccttcggggtgagcccccagagctggacctgagctattctcactcggacctggggaaacgg

SEQ ID NO: 730 - DNA, 210 bp, Homo sapiens
ccatcacctggaggacttctacccggaacatcagcagcgaagaaaaggcttcgtggactcgaccagagaagcaagagactctggatgggcacatggtggtgcgtagccatgcccgtgtgtcgtcgctgaccctgaagagcatccagtacactgatgccggagagtacatctgcaccgccagcaacaccatcggccaggactcccagtcca

SEQ ID NO: 731 - DNA, 210 bp, Homo sapiens
cgggatcttcctgattttcatcgagattgcctacaagcggcacaaggatgctcgccggaagcagatgcagctggcctttgccgccgttaacgtgtggcggaagaacctgcagcagtaccatcccactgatatcacgggcccgctcaacctctcagatccctcggtcagcaccgtggtgtgaggcccccggaggcgcccacctgcccagtt

SEQ ID NO: 732 - DNA, 350 bp, Homo sapiens
gccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagt

SEQ ID NO: 733 - DNA, 210 bp, Homo sapiens
tgactgcatcgttgataaaatccgcagaaaaaactgcccagcatgtcgccttagaaagtgctgtcaggctggcatggtccttggaggttttcgaaacttacatattgatgaccagataactctcattcagtattcttggatgagcttaatggtgtttggtctaggatggagatcctacaaacacgtcagtgggcagatgctgtattttgc

SEQ ID NO: 734 - DNA, 350 bp, Homo sapiens
tgactgcatcgttgataaaatccgcagaaaaaactgcccagcatgtcgccttagaaagtgctgtcaggctggcatggtccttggaggttttcgaaacttacatattgatgaccagataactctcattcagtattcttggatgagcttaatggtgtttggtctaggatggagatcctacaaacacgtcagtgggcagatgctgtattttgcacctgatctaatactaaatgattcctttggaagggctacgaagtcaaacccagtttgaggagatgaggtcaagctacattagagagctcatcaaggcaattggtttgaggcaaaaaggagttgtgtcgagctcacagcgt

SEQ ID NO: 735 - DNA, 420 bp, Homo sapiens
gccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaagg

SEQ ID NO: 736 - DNA, 980 bp, Homo sapiens
tatggcccgataaaagtgaaggatgtaatagatcgtggcccttcaatttagaagagattaagaaaaattggatggagattacagacagttcactcccttccccctcaactctcccaatcattaacatcttctatagtgtgttacatttgttacaattaatgaactgatactgatactttattattaaataaagtttagcattaacattagggtttactcctgtgttgtgcggctttggacaaatgcaggagagcaagtcccacccagtgtgctctggagcagccgctggccctaaaccccctgagccatacctccccttcttcctccccttgaacccccaagcaaccgcgaatctcattcctgtctcttaagactaccttttccaaattgtcacgtcgttggaatcatacagtatgtagcctctgcagactggcttcttgcacttagcaatgtatgtttgcagttcctccagtgtcttttcatgactcgacggctcattggtttttgttgctgaaaatattccattgtttggatgtacactttatcccttcacctataacagcttgtattttcgtgtgcagttttatgattactcaaattgcacttgtagatatatcttaacaaacacttcatacaaaataagcatagtattattttattcaccaaagtattgttaattagcagagctcaattctttggtgtcagtttatcaaatttaccttctaggttttgagtttattattaagaacctgcgtagacttattttattttttaatgcataggatcttttgccagaaatgagggcatactggcctgacgtaattcactcgtttcccaatcgcagccgcttctggaagcatgagtgggaaaagcatgggacctgcgccgcccaggtggatgcgctcaactcccagaagaagtactttggcagaagcctggaactctacagggagctggacctcaacagtgtgcttctaaaa

SEQ ID NO: 737 - DNA, 910 bp, Homo sapiens
cgtgtggaaccaaacctgcgcgcgtggccgggccgtgggacaacgaggccgcggagacgaaggcgcaatggcgaggaagttatctgtaatcttgatcctgacctttgccctctctgtcacaaatccccttcatgaactaaaagcagctgctttcccccagaccactgagaaaattagtccgaattgggaatctggcattaatgttgacttggcaatttccacacggcaatatcatctacaacagcttttctaccgctatggagaaaataattctttgtcagttgaagggttcagaaaattacttcaaaatataggcatagataagattaaaagaatccatatacaccatgaccacgaccatcactcagaccacgagcatcactcagaccatgagcgtcactcagaccatgagcatcactcagaccacgagcatcactctgaccataatcatgctgcttctggtaaaaataagcgaaaagctctttgcccagaccatgactcagatagttcaggtaaagatcctagaaacagccaggggaaaggagctcaccgaccagaacatgccagtggtagaaggaatgtcaaggacagtgttagtgctagtgaagtgacctcaactgtgtacaacactgtctctgaaggaactcactttctagagacaatagagactccaagacctggaaaactcttccccaaagatgtaagcagctccactccacccagtgtcacatcaaagagccgggtgagccggctggctggtaggaaaacaaatgaatctgtgagtgagccccgaaaaggctttatgtattccagaaacacaaatgaaaatcctcaggagtgtttcaatgcatcaaagctactgacatctcatggcatgggcatccaggttccgctgaatgcaacagagttc

SEQ ID NO: 738 - DNA, 350 bp, Homo sapiens
gcagggagacttcaagcgccaagctgacctttggaggtcaggacggacccagaatcaggcaggaatttggcaggcccgcggcggcgtaggacggaggcgtcgctagggtcttgttctcttggccaggctggagtgctgtgggaaaatctgggctcactgcagcctcaacctccgggactcaagtgatcatcctgcctcagccaccccagagtagctgagaatacaggcgtgcgccaccaggctcgggcagcttcgaaccagtgcaatgacgatgccagtcaacggggcccacaaggatgctgacctgtggtcctcacatgacaagatgctggcacaacccctcaaagaca

SEQ ID NO: 739 - DNA, 100 bp, Homo sapiens
gtaaaagacagctattttcaggcacggtttctcgtgtgctttaattacagaaagcactccaaagacctccgccagctgcagccctgcccctgagtccccg

SEQ ID NO: 740 - DNA, 700 bp, Homo sapiens
cccaagggtgggtgccctaaagcaccacagcaggaagagcttcccctcagcagcgacatggtggagaagcagactgggaaaaagatttttccaaaagaatcgtgatctcagtgacatatacgtggaagatggaaatggagcccacgactctgcagtgcatcctgatgccgcgctgacctgacggcttgtgcgtgtccctttggctgcaccagtgagcacagtggcaggcgtgtcagagaaagggccccttctgcagacggtctctcaccattgccgaccacggaatcccagaaccgctgagctgcctcgggaagaaccagcaggtgtctgcatcgttgagtgtgttctgatccaaaggataaagataaagtttctctaaccaagaccccaaaactggagcgtggcgatggcgggaaggaggtgagggagcgagccagcaagcggaagctgcccttcaccgcgggcgccaatggggagcagaaggactcggacacagatgcctccagcccagtccctgttgtggtgctgcaaggctggtacgctcctcgaagcaccatggcatgagatggaggttcctagaagcaagaagaaagagaagcagggccctgagcggaagaggattaagaaggagcctgtcacccggaaggccgggctgctgtttggcatggggctgtctggaatccgagccggctaccccctc

SEQ ID NO: 741 - DNA, 210 bp, Homo sapiens
tgatcaagctatcattcttgatggtataaaaatggacactggagtagaagtctctgatattggaagccaagagctggggtttcaccatgttggccagactggactcgagttcctgacctcagatgctcccataatactctcagatagtgaagaagaagaaatgatcattttggaaccagacaagaatccaaagaaaataagaacacagac

SEQ ID NO: 742 - DNA, 2380 bp, Homo sapiens
ctttctgccgccatcttggttccgcgttccctgcacaaaatgcccggcgaagccacagaaaccgtccctgctacagagcaggagttgccgcagccccaggctgagacagctgtgctacctatgtcttcagccttgagtgtcactgctgccttagggcagcctggacctaccctcccccctccttgctctcctgccccacaacagtgccctctctcagctgctaaccaggcttccccattcccttccccctctactattgcctcgacccctttagaagttccttttccccagtcatcctctggaacagccctacctttgggaactgcccctgaagccccaaccttcctaccaaacctaatagggcctcccatctccccagctgccttagctctagcctctcccatgatagctccaactctgaaagggaccccttcctcttcagctcccttagctctggttgccctggctccccactcagttcagaagagttctgcttttccacctaaccttcttacttcacctccttcagtggctgtagctgagtcagggtcagtgataactctgtcagctcccattgctccctcagaaccaaagactaatcttaataaagttccctctgaggtagtccctaatccaaaaggcacccccagccctccatgtatagtcagtactgttccttaccactgtgtgactcccatggcctctattcaatctggagtggcctcccttcctcagacaacacccacaactaccctagccatcgcttcccctcaagtcaaagataccaccatttcctcagttctgatttctccacaaaacccaggaagcctcagcctgaaggggcctgttagtccacctgctgccttatctctttcaactcagtctcttcctgtggtgacctcttctcaaaagactgcgggtcccaacacccccccagattttcccatttctctgggctctcatcttgcacctttacatcagagttcttttggttctgtccaacttttaggtcaaacaggtcctagtgctttgtcagaccccacagagaagaccatttctgtagatcattcttccacaggggcctcttatccttctcagagatctgtaattcctccccttccttccagaaatgaggtagttcctgctactgtggctgcctttccagtggtggctccatctgttgacaaaggtccctctaccatctctagcataacctgcagcccttctggctccttaaatgtagctacctcttcttcattatctcctacaacctctctcattctcaaaaactctcctaatgccacttatcattatcctttagtggcccaaatgcccgtttcttctgttggaaccaccccacttgtggtgactaacccctgtacaattgctgcagcacctactactacctttgaggtagctacttgtgtttctcctccaatgtcatcaggtcccataagtaacatagaaccaacttcccctgctgccttggttatggcacctgtggctcccaaagagccttctactcaagtagcaaccactctgaggataccagtctctcctcctctgccagaccctgaagacctcaaaaatctctccagttcagtattggttaaatttccaacacaaaaagacctccaaactgtacctgcctctcttgaaggagcccctttctctccagcccaagcaggactcaccaccaagaaagaccctactgtattaccgttagtccaggcagcccctaaaaattccccttctttccaaagtacatcctcttctccagagatacctctttctcctgaagccaccctagcaaagaaaagccttggggagcctctccctatagtggctgcatttcctttggaaagtgctgaccctgccggggtggctcccacaactgccaaagcagctgcctttgagaaggtccttcctaaacctgaatcagcatctgtctctgcagcacccaccccaccagtctctctgcctcttgctccctccccagttcccactctgcctcctaaacagcaatttctgccgtcctctcctgggctggtgttggaatcaccctctaaaccccttgcccctgctgatgaggatgagctgccgcctctgattcccccggaaccaatctctgggggagtgcctttccagtcggtcctcgtcaacatgcccacccctaaatctgctggaatccctgtcccaaccccctctgccaagcaacctgttacgaagaacaacaaggggtctggaacagaatctgacagtgatgaatcagtaccagagcttgaagaacaggattccacccaggcaaccacacaacaagcccagctggcggcagcagctgaaatcgatgaagaaccagtcagtaaagcaaaacagagtc

SEQ ID NO: 743 - DNA, 140 bp, Homo sapiens
tgcagccggagttcaaacctaagcagctggaaggaaccatggccaactgtgagcgtaccttcattgcgatcaaaccagatggggtccagcggggtcttgtgggagagattatcaagcgttttgagcagaaaggattccgc

SEQ ID NO: 744 - DNA, 210 bp, Homo sapiens
  misc_feature (77)..(77): n is a, c, g, or t
gaagagcacttcagggatgatgatgagggtccagtgtccaaccagggctacatgccttatttaaacaggttcatttnggaaaagatgaatacctgcttaagaagcttacagaagctatgggaggaggntggcagcaagaacaatttgaacattataaaatcaactttgatgacagtaaaaatggcctttctgcatgggaacttattgagc

SEQ ID NO: 745 - DNA, 210 bp, Homo sapiens
cagggggaagcaaacctctcaccttccaaatccagggcaacaagctgactttgactggtgcccaggtgcgccagcttgctgtggggcagccccgcccgctgcaaatgccaccaaccatggtgaataatacaggcgtggtgaagattgtagtgagacaagcccctcgggatggactgactcctgttcctccattggccccagcaccccggc

SEQ ID NO: 746 - DNA, 210 bp, Homo sapiens
tccggaactgctcccggcattcctcgcgagtgtatggcgtgggctcccttccccctctgtgggtcccgcgaggagactctcgggctttgaggtgtgcctgcacaggagacagcaccagccaagctgattgtgtatctacagcgtttccggcctcaagactatcagcgcctgctagaagtgaacagctccagagagaggccacaggagact

SEQ ID NO: 747 - DNA, 480 bp, Homo sapiens
ctattgaacatgctagggctcggtcacgaggtggaagaggtagaggacgatactctgaccgttttagtagtcgcagacctcgaaatgatagacggtatgtgaagggtggatggctgcattgaacaattattgtaggggtagcatttaagattcaggagtcattagcagtgatgattttgggacctgccgtataatctgttcttctattcccacgttagccaattgttcttgatgaatctatatgagtcatagaacacaaatctattgacggaagtcattagaatggcttgtgatatctgatggcttgaacttgcccacagttgaacacaagtgctgtcattgcatttcttccattgtgaatacgaattttcttcctcagaaatgctccacctgtaagaacagaaaatcgtcttatagttgagaatttatcctcaagagtcagctggcaggatctcaaagatttcatgagacaagctgggg

SEQ ID NO: 748 - DNA, 210 bp, Homo sapiens
gcgagtacgtcatcgtgccctccacctacgagccccaccaggagggggaattcatcctccgggtcttctctgaaaagaggaacctctctgaggaagttgaaaataccatctccgtggatcggccagtgcccatcatcttcgtttcggacagagcaaacagcaacaaggagctgggtgtggaccaggagtcagaggagggcaaaggcaaaa

SEQ ID NO: 749 - DNA, 360 bp, Homo sapiens
  misc_feature (101)..(101): n is a, c, g, or t
actggaaggtctttgagagctggatgcaccattggctcctgtttgaaatgagcaggcactccttggagcaaaagcccactgacgctccaccgaaagtactnaccaagtgccaggaagaggtcagccacatccctgctgtccacccgggttcattcaggcccaagtgcgacgagaacggcaactatctgccactccagtgctatgggagcatcggctactgctggtgtgtcttccccaacggcacggaggtccccaacaccagaagccgcgggcaccataactncagtgagtcactggaactggaggacccgtcttctgggctgggtgtgaccaagcaggatctgggcccagtccccatgt

SEQ ID NO: 750 - DNA, 350 bp, Homo sapiens
actacaactcactgacccgctcagaacactcacactcgaccacactgccgagggactactccaccctcacctccgtctcctcccacggcctccctcccatctgggaacacgggaggagcaggcttccgctgtcctgggccctggggtcccggagtcgggctcagatgaaagggttccccccttccaggggcccacgagactctataatcctggctgggaggccagcagcgccctcctggggcccagactctcgcctgactgctggtgtgcccgacacgcccacccgcctggtgttctctgccctggggcccacatctctcagagtgagctggcaggagccgcggtgcgag

SEQ ID NO: 751 - DNA, 210 bp, Homo sapiens
gacctttctgaaagggaagagagttggctactggctgagcgagaagaaaatcaagaagctgaatttccaggccttcgccgagctgtgcaggaagcgagggatggaggttgtgcagctgaaccttagccggccgatcgaggagcagggccccctggacgtcatcatccacaagctgactgacgtcatccttgaagccgaccagaatgatag

SEQ ID NO: 752 - DNA, 280 bp, Homo sapiens
agcacatgctgggctcgggggcgatgggcttgtgcgcggacctggcgacgctctagccccgagccgcgtattcgtggccgggtcctccctgggaacagggtgaaggccgagaacctctggcctcaggaagcgcatgcgcaaccggttctccgaaacatggagtcctgtaggcaaggtcttacctgaatcaggatgagggagtggtgggtccaggtggggctgctggccgtgcccctgcttgctgcgtacctgcacatcccaccccctcagctctcccctg

SEQ ID NO: 753 - DNA, 180 bp, Homo sapiens
agaatgtttttgaccagaaaaccgacaaccttcccagaaagtccaagctcgtggtgggtggaaaagtgttcgccgagggtctgcttggccactcagtgcagctgcgattaaccctaaaggctttaaggaacgggccacctgtaacagagacaccagccttcctgtatagacactaaattg

SEQ ID NO: 754 - DNA, 660 bp, Homo sapiens
  misc_feature (643)..(643): n is a, c, g, or t
gaagatggcggcccgggcgggtttccagtctgtggctccaagcggcggcgccggagcctcaggaggggcgggcgcggctgctgccttgggcccgggcggaactccggggcctcctgtgcgaatgggcccggctccgggtcaagggctgtaccgctccccgatgcccggagcggcctatccgagaccaggtatgttgccaggcagccgaatgacacctcagggaccttccatgggaccccctggctatggggggaacccttcagtccgacctggcctggcccagtcagggatggatcagtcccgcaagagacctgcccctcagcagatccagcaggtccagcagcaggcggtccaaaatcgaaaccacaatgcaaagaaaaagaagatggctgacaaaattctacctcaaaggattcgtgaactggtaccagaatcccaggcctatatggatctcttggcttttgaaaggaaactggaccagactatcatgaggaaacggctagatatccaagaggccttgaaacgtcccatcaagtcagccttgtccaaatatgatgccactaaacaaaaagaggaagttctcttccttttttaagtcccttggtgattgaactggacaaagacctgtatgggccagacaacncatctggtagaatggca

SEQ ID NO: 755 - DNA, 180 bp, Homo sapiens
cctggacacgctggtggtgctgcaccgggccggggcgcggctggacgtgcgcgatgcctggggccgtctgcccgtggacctggctgaggagctgggccatcgcgatgtcgcacgacatccccgattgaaagaaccagagaggctctgagaaacctccggaaacttagatcatcagtcacc

SEQ ID NO: 756 - DNA, 180 bp, Homo sapiens
gggcacgaggctgctgtgaagctgaaaccggagccggtccgctgggcggcgggcgccgggggccggaggggcgcgcgcggcggcggcaccccagcgtttaggcgcggaggcagccatggcgggcaacttcgactcggaggagcggagtagctggtactgggggaggttgagtcggcagga

SEQ ID NO: 757 - DNA, 239 bp, Homo sapiens
ggacgatcacaccaaggcacaagagggagaacagcccgtgaggccatttcccgaccgggagggattgtgcccccacaacgacattagtccagaccgaatgccggttcattcccaaaggccccaagcactggaccacagaggtacggatacatacgactccaacacggagaagctcatcaggacacgggcgccgaaggacccaaagaccatccagggatccgtaccccatccgccaggaa

SEQ ID NO: 758 - DNA, 180 bp, Homo sapiens
ctgcaggacctcagctcttgcatcacccaggggaaagatgcagctgtatccaagaaagccagcccagaggctgccagcactcccagggaccctattgacgttgacctggatgtctccaatacaacgacagcccagaagaggaagtgcagccagacccagtgccccaggaaggtcatcaag

SEQ ID NO: 759 - DNA, 180 bp, Homo sapiens
accagccgcagaggatggcgcctgtgggcacagacaaggaggctcagtgacctcctggacttcagcatgatgttcccgctgcctgtcaccaacgggaagggccggcccgcctccctggccggggcgcagttcggaggttcaggcaagagcggtgagcggggcgcctatgcctccttcggg

SEQ ID NO: 760 - DNA, 180 bp, Homo sapiens
gagtttcgggatgtccggatgcctgtggccaaccccttccccaaggagcgggcactcccatgtgatagtgccaggccagtccctggtgagtacagccacccatggagcctgagaaccttgacctccagtccccaaccaagctgagtgccagcggggaggactccaccatcccacaagcca

SEQ ID NO: 761 - DNA, 180 bp, Homo sapiens
gggggcggcccggcggagaccacctggctgggagaaggcggaggaggcgatggctactatccctcgggaggcgcctggccagagcctggtcgagccggaggaagccaccagagtttgaattcttatacaaatggagcgtatggtccaacataccccccaggccctggggcaaatactgcc

SEQ ID NO: 762 - DNA, 225 bp, Homo sapiens
ggaatctgtatattgccaaagtagaaaaatcagatgttgggaattatacctgtgtggttaccaataccgtgacaaaccacaaggtcctggggccacctacaccactaatattgagaaatgatgtccagtaccaactattatctggcgaagagctgatggaaagccaatagcaaggaaagccagaagacacaagtcaaatggaattcttgagatccctaattttca

SEQ ID NO: 763 - DNA, 225 bp, Homo sapiens
cattacaactccatcaaagcccagctggcacctctcaaacctgaatgcaactaccaagtacaaattctacttgagggcttgcacttcacagggctgtggaaaaccgatcacggaggaaagctccaccttaggagaagggaaatatgctggtttatatgatgacatctccactcaaggctggtttattggactgatgtgtgcgattgctcttctcacactactatt

SEQ ID NO: 764 - DNA, 225 bp, Homo sapiens
caataaaactcagtcttgatttctgattatgtgaaaaaatttggagaaaattttgcatcatgtcaagctggaatatccagtttttacacaaaggatttaattgtgatgggggccccaggatcatcttactggactggctctctttttgtctacaatataactacaaataaatacaaggcttttttagacaaacaaaatcaagtaaaatttggaagttatttagga

SEQ ID NO: 765 - DNA, 225 bp, Homo sapiens
gctcagggaagcaggagatcacgctgcccccgtctcgtaagaccgaacttgtagttgaagttaagtcagataagctcccagaagagatgggcctcctgcagggcagcagcggtgacaagagggctccgggagaccagccctgaatgtcctcgtgaccccggagctgttggagacaggtgttgaatgcacggcctccaacgacctgggcaaaaacaccagcatcct

SEQ ID NO: 766 - DNA, 225 bp, Homo sapiens
ctgtagccatcccctggccagcttcagctttacctctgcatgtaccttcatctgctcagaaggaactgagttaattgggaagaagaaaaccatttgtgaatcatctggaatctggtcaaatcctagtccaatatgtcaaagcaagaaatccaagagaagtatgaatgacccatattaaatcgcccttggtgaaagaaaattcttggaatactaaaaatcatgaga

SEQ ID NO: 767 - DNA, 1132 bp, Homo sapiens
agctcctgtggtggtagcagcggtagcgggagacggagcgagtccagcggccgcgggcagacccggagggaacggaggaagcggtcatgtctcgctacacgaggccccccaacacctccctgttcatcaggaacgtcgcggacgccaccagaagatctaaagcagtccacagtagctggcaagcaccccccagtttgaaccaacctgttagctagaatccaagcataaacccagcaggcgagacaaaaggcacctaaagttcaagcatcaaggagtaaagagggagggtggacacagatataaagacctggaagaggggaagtctttatcaagcaaaagacaaagccaacaccaggttgagacttcggctttcctacatttactcagagttccagagtcaaagccaagtctgattttgttggttctgcgtctcttataaagtccatcttgcaagccttaaagagtaaaggtcaaggttcaagatcaagtgacattgagatttgaagatgttcgaggtgctgaagatgctctttataacctcaatagaaagtgggtatgtggccgtcagattgaaatacagtttgcacaaggtgatcgcaaaacaccaggccaaatgaaatcaaaagaacgtcatccttgttctccaagtgatcacaggagatcaagaagccccagccaaagaagaactcgaagtagaagttcttcatggggaagaaataggaggcggtcagacagccttaaagagtctcgacacaggcgattttcttatagcaagtctaaatctcgttccaaatcattaccaaggcggtctacctcagcaaggcagtcaagaactccaagaaggaattttggctctagaggacggtcaaggtccaagtccttacaaaagaggtccaagtcaataggaaaatcacagtcaagttcacctcaaaagcagactagctcaggaacaaaatcaagatcacatggaagacattctgactcaatagcaagatccccgtgtaaatctcccaaagggtataccaattctgaaactaaagtacaaacagcaaagcattctcattttcggtcacattccagatctcgaagttatcgtcataaaaacagttggtgaacagcaacagaaagagca

SEQ ID NO: 768 - DNA, 1813 bp, Homo sapiens
atgtcccctccaggttaagaaagccgaaccagagccgatgcgagaggaggagaaaatgattcctcctacgaaacctgaaattcaggccaaggctccaagtagtctgagtgatgctgtcccccagcgagcagatcacagggtagtgggcaccatcgaccagcttgtgaaacgtgtcatcgaaggcagcctgtctcccaaagagagaactcttctcaaagaggaccctgcttactggtttttgtctgatgaaaatagtctggagtataaatattacaagctgaagttggcagaaatgcagcggatgagcgagaacttgcgaggagccgaccagaagccgacctcagcagactgtgcagtgagggccatgctgtactcccgggctgtccgcaacctcaagaagaaactccttccgtggcagcggcgggggctcctccgtgctcaagggctccggggctggaaggcgaggagagcgaccaccgggacccagaccctcctatcctcaggcaccaggctgaaacaccacggccggcaggctccaggcctctcacaggcaaaaccatccctgccagacagaaatgatgctgccaaggactgcccgccagacccagttggaccttctcctcaggaccccagcttagaagcctcaggcccatcccccaagccagcaggagtggacatctctgaagcacctcagacctcttctccctgcccatctgctgacattgacatgaagacaatggagactgcagagaaactggctagatttgttgctcaggtgggaccagagatcgaacaattcagcatagaaaacagcaccgataaccctgacctgtggtttctacatgaccaaaatagttctgctttcaaattctatcgaaagaaagtgtttgaactatgtccatcaatttgtttcacgtcatctccgcacaaccttcacactggtggtggtgacaccacgggttctcaggagagccccgtggacctcatggaaggggaagcagagtttgaagacgagccccctccgcgggaggctgagctggagagcccagaggtgatgcctgaggaggaggacgaggacgatgaggatgggggagaggaggcccccgctcctggaggggcgggcaagtctgagggcagcacccctgccgacggccttcccggcgaggctgccgaggacgacctggctggagcacctgccttgtcacaggcctcctcaggtacctgcttccctcggaagaggatcagcagcaagtcattgaaggttggcatgattccagctcccaagagagtgtgtctcatccaggagccaaaagtccatgaaccagttcgaattgcctatgacaggcctcggggtcgtcccatgtccaaaaagaagaaacccaaggacttggacttcgcccagcagaagctgaccgataagaacctgggcttccagatgctgcagaagatgggctggaaggagggccatggcctgggctccctcggaaagggcatcagggagccggtcagcgtgggaaccccctcggaaggggaagggttgggtgctgacgggcaggagcacaaagaagacacattcgatgtgttccgacagaggatgatgcagatgtacagacacaagcgggccaacaaatagatcaaaaccactgatgtgaaagataagccttgaagcagcaattgcccttaaaacatcatccctgccctggatcggcctggagccagtgcccaattccagggtcacccccgagaggacaacaggcatctggaagtgctctctcgccactctgggtgctttactgtctctggcttgtttcca

SEQ ID NO: 769 - DNA, 2480 bp, Homo sapiens
atgtcccctccaggttaagaaagccgaaccagagccgatgcgagaggaggagaaaatgattcctcctacgaaacctgaaattcaggccaaggctccaagtagtctgagtgatgctgtcccccagcgagcagatcacagggtagtgggcaccatcgaccagcttgtgaaacgtgtcatcgaaggcagcctgtctcccaaagagagaactcttctcaaagaggaccctgcttactggtttttgtctgatgaaaatagtctggagtataaatattacaagctgaagttggcagaaatgcagcggatgagcgagaacttgcgaggagccgaccagaagccgacctcagcagactgtgcagtgagggccatgctgtactcccgggctgtccgcaacctcaagaagaaactccttccgtggcagcggcgggggctcctccgtgctcaagggctccggggctggaaggcgaggagagcgaccaccgggacccagaccctcctatcctcaggcaccaggctgaaacaccacggccggcaggctccaggcctctcacaggcaaaaccatccctgccagacagaaatgatgctgccaaggactgcccgccagacccagttggaccttctcctcaggaccccagcttagaagcctcaggcccatcccccaagccagcaggagtggacatctctgaagcacctcagacctcttctccctgcccatctgctgacattgacatgaagacaatggagactgcagagaaactggctagatttgttgctcaggtgggaccagagatcgaacaattcagcatagaaaacagcaccgataaccctgacctgtggtttctacatgaccaaaatagttctgctttcaaattctatcgaaagaaagtgtttgaactatgtccatcaatttgtttcacgtcatctccgcacaaccttcacactggtggtggtgacaccacgggttctcaggagagccccgtggacctcatggaaggggaagcagagtttgaagacgagccccctccgcgggaggctgagctggagagcccagaggtgatgcctgaggaggaggacgaggacgatgaggatgggggagaggaggcccccgctcctggaggggcgggcaagtctgagggcagcacccctgccgacggccttcccggcgaggctgccgaggacgacctggctggagcacctgccttgtcacaggcctcctcaggtacctgcttccctcggaagaggatcagcagcaagtcattgaaggttggcatgattccagctcccaagagagtgtgtctcatccaggagccaaaagtccatgaaccagttcgaattgcctatgacaggcctcggggtcgtcccatgtccaaaaagaagaaacccaaggacttggacttcgcccagcagaagctgaccgataagaacctgggcttccagatgctgcagaagatgggctggaaggagggccatggcctgggctccctcggaaagggcatcagggagccggtcagcgtgtacgcagcaggcagcctggggtgggagtgggtggggcctcagtccttccacctgcagcctgccgcttggctccttcacagccaagatggcttacagctggcagttgatttttgttttttaaacagaaggcatcttcagatgagaagctgatcatttacatgtgcaggtgtttacagggctcctttctgtcctggtgtagattttttaaccagcttgttggccctggtcattttggccacatttgtgaccatcataaaagctaagtggtatttctgtgtagtttccgtctggaactgctttcccattcccgggaacccatagccgggccagccagggtcccgaacacaggcccaaagtttattaaaccccgatcataacctccagcaggcatttcatttaatactgagcttagttcctgctgggtaaggcattccgaggtaaccagggccctctgggcaccccctcaaaagccagctcttcgagggtgagtactccttgtttctactgtgagtcgcgtcttgattttccctttctttgatgtctcagtgtgtgtcccaaacacctgcatctcatggactgtttgtgcccatgcccagttcctggcatgccaggccctgggctcaggtgcacaactgactctctttttcactccctaggggaaccccctcggaaggggaagggttgggtgctgacgggcaggagcacaaagaagacacattcgatgtgttccgacagaggatgatgcagatgtacagacacaagcgggccaacaaatagcaaaccgtacttgggcactggctccaggccgatccagggcagggatgatgttttaagggcaattgctgcttcaaggcttatctttcacatcagtggttttgatttccagggtcacccccgagaggacaacaggcatctggaagtgctctctcgccactctgggtgctttactgtctctggcttgtttcca

SEQ ID NO: 770 - DNA, 1698 bp, Homo sapiens
ctaatgctcagcgatcaggactgaaccagattcccaatcgtagattcaccctctggtggtccccgaccattaatcgagccaatgtatatgtaggctttcaggtgcagctagacctgacgggtatcttcatgcacggcaagatccccacgctgaagatctctctcatccagatcttccgagctcacttgtggcagaagatccatgagagcattgttatggacttatgtcaggtgtttgaccaggaacttgatgcactggaaattgagacagtacaaaaggagacaatccatccccgaaagtcatataagatgaactcttcctgtgcagatatcctgctctttgcctcctataagtggaatgtctcccggccctcattgctggctgactccaagtaagtgcctcaggacccagccctaggcagccaggacactttcgttttcctgttcttctagccctgcaactttaggaattgtcctgtctgcctttgtttcaaacttggagccagtgctacgcttggagcctgtcaacacccttagtcagatctgctgattctctggggtcctgctgacctggaacaagttggtggagtgggtgggatggttttgggatttaagtggttctggttctggggacattggttatgcccatggtttcttagaagcttgaaccctcttcatcctcagggatgtgatggacagcaccaccacccagaaatactggattgacatccagttgcgctggggggactatgattcccacgacattgagcgctacgcccgggccaagttcctggactacaccaccgacaacatgagtatctacccttcgcccacaggtgtactcatcgccattgacctggcctataacttgcacagtgcctatggaaactggttcccaggcagcaagcctctcatacaacaggccatggccaagatcatgaaggcaaaccctgccctgtatgtgttacgtgaacggatccgcaaggggctacagctctattcatctgaacccactgagccttatttgtcttctcagaactatggtgagctcttctccaaccagattatctggtttgtggatgacaccaacgtctacagagtgactattcacaagacctttgaagggaacttgacaaccaagcccatcaacggagccatcttcatcttcaacccacgcacagggcagctgttcctcaagataatccacacgtccgtgtgggcgggacagaagcgtttggggcagttggctaagtggaagacagctgaggaggtggccgccctgatccgatctctgcctgtggaggagcagcccaagcagatcattgtcaccaggaagggcatgctggacccactggaggtgcacttactggacttccccaatattgtcatcaaaggatcggagctccaactccctttccaggcgtgtctcaaggtggaaaaattcggggatctcatccttaaagccactgagccccagatggttctcttcaacctctatgacgactggctcaagactatttcatcttacacggccttctcccgtctcatcctgattctgcgtgccctacatgtgaacaacgatcgggcaaaagtgatcctgaagccagacaagactactattacagaaccacaccacatctggcccactctgactgacgaagaatggatcaaggtcgaggtgcagctcaaggatctgatc

SEQ ID NO: 771 - DNA, 1619 bp, Homo sapiens
ctaatgctcagcgatcaggactgaaccagattcccaatcgtagattcaccctctggtggtccccgaccattaatcgagccaatgtatatgtaggctttcaggtgcagctagacctgacgggtatcttcatgcacggcaagatccccacgctgaagatctctctcatccagatcttccgagctcacttgtggcagaagatccatgagagcattgttatggacttatgtcaggtgtttgaccaggaacttgatgcactggaaattgagacagtacaaaaggagacaatccatccccgaaagtcatataagatgaactcttcctgtgcagatatcctgctctttgcctcctataagtggaatgtctcccggccctcattgctggctgactccaagtaagtgcctcaggacccagccctaggcagccaggacactttcgttttcctgttcttctagccctgcaactttaggaattgtcctgtctgcctttgtttcaaacttggagccagtgctacgcttggagcctgtcaacacccttagtcagatctgctgattctctggggtcctgctgacctggaacaagttggtggagtgggtgggatggttttgggatttaagtggttctggttctggggacattggttatgcccatggtttcttagaagcttgaaccctcttcatcctcagggatgtgatggacagcaccaccacccagaaatactggattgacatccagttgcgctggggggactatgattcccacgacattgagcgctacgcccgggccaagttcctggactacaccaccgacaacatgagtatctacccttcgcccacaggtgtactcatcgccattgacctggcctataacttgcacagtgcctatggaaactggttcccaggcagcaagcctctcatacaacaggccatggccaagatcatgaaggcaaaccctgccctaactatggtgagctcttctccaaccagattatctggtttgtggatgacaccaacgtctacagagtgactattcacaagacctttgaagggaacttgacaaccaagcccatcaacggagccatcttcatcttcaacccacgcacagggcagctgttcctcaagataatccacacgtccgtgtgggcgggacagaagcgtttggggcagttggctaagtggaagacagctgaggaggtggccgccctgatccgatctctgcctgtggaggagcagcccaagcagatcattgtcaccaggaagggcatgctggacccactggaggtgcacttactggacttccccaatattgtcatcaaaggatcggagctccaactccctttccaggcgtgtctcaaggtggaaaaattcggggatctcatccttaaagccactgagccccagatggttctcttcaacctctatgacgactggctcaagactatttcatcttacacggccttctcccgtctcatcctgattctgcgtgccctacatgtgaacaacgatcgggcaaaagtgatcctgaagccagacaagactactattacagaaccacaccacatctggcccactctgactgacgaagaatggatcaaggtcgaggtgcagctcaaggatctgatc

SEQ ID NO: 772 - DNA, 601 bp, Homo sapiens
agtctcgagggaagacagaggagtcgggggaggatcggggcgatggtccgccagacagagaccccacgctttctccttctgcctttatcctgcgagccatccagcaggctgtgggaagctccctgcagggggacctgcccaatgataaagatggctctcggtgtcatggccttcgatggcggcgctgccggagtccacggtcagagccccgttcccaggaatcagggggcactgacacggctactgtgttggacatggccacggacagcttcctcgcagggctggtgagtgtcctggatcccccggatacctgggttcccagccgcctggacctgcggcctggcgaaagtgaggacatgctggagctggtggctgaggtccgaatcggggacagagatcccatccctctgcctgtgcccagcctgctgccccgtctcagggcctggaggacgggcaaaacggtttctccacagtcgaactcctctaggcccacctgtgcccgtcacctcaccttgggcacgggagacgggggccctgcaccgccccctgcacccccagccccacctgccccccgattcgatatctatgaccccttccaccc

SEQ ID NO: 773 - DNA, 1005 bp, Homo sapiens
agtctcgagggaagacagaggagtcgggggaggatcggggcgatggtccgccagacagagaccccacgctttctccttctgcctttatcctgcgagccatccagcaggctgtgggaagctccctgcagggggacctgcccaatgataaagatggctctcggtgtcatggccttcgatggcggcgctgccggagtccacggtcagagccccgttcccaggaatcagggggcactgacacggctactgtgagtaagaagagggggctgggggcctggctcacgggtatcagggaggaagggatgggggcctgagtctgggggaatggggtttggggacctggactcctggctctgcgatgctgaccaggggcaatgttggagagtctgggggcctgatctgtgggcctgagctttgagtgttgatggcagtcaggctataggaattagatcctcagttttcttggggatcttagatgtctgggttcctgagaggttagggagtggggaagcaggatttgccagtcttcatgtgaccagggacggcgtagagcctctctggcctcttccaggtgttggacatggccacggacagcttcctcgcagggctggtgagtgtcctggatcccccggatacctgggttcccagccgcctggacctgcggcctggcgaaagtgaggacatgctggagctggtggctgaggtccgaatcggggacagagatcccatccctctgcctgtgcccagcctgctgccccgtctcagggcctggaggacgggcaaaacggtttctccacagtcgaactcctctaggcccacctgtgcccgtcacctcaccttgggcacgggagacgggggccctgccccaccccctgccccctcctctgcatcctcctccccttccccttctccctcatcttcctccccttcccctcccccacccccaccgccccctgcacccccagccccacctgccccccgattcgatatctatgaccccttccaccc

SEQ ID NO: 774 - DNA, 1242 bp, Homo sapiens
ccaaagccctctctttattggctcctgctccaaccatgacaagtctgatgcctggtgcaggattgcttccaataccgaccccaaatcctttgactactcttggtgtttcacttagcagtttgggagctataccagcagcagcactagaccccaacattgcaacacttggagagataccacagccaccacttatgggaaacgtggatccttccaaaatagatgaaattaggagaacggtttatgttggaaatctgaattcccagacaacgacagctgatcaactacttgaattttttaaacaagttggagaagtgaagtttgtgcggatggcaggtgatgagactcagccaactcggtttgcttttgtggaatttgcagaccaaaattctgtaccaagggcccttgcttttaatggagttatgtttggagacaggccactgaaaataaatcactccaacaatgcaatagtaaaaccccctgagatgacacctcaggctgcagctaaggagttagaagaagtaatgaagcgagtacgagaagctcagtcatttatctcagcagctattgaaccagagtctggaaagagcaatgaaagaaaaggcggtcgatctcgttcccatactcgctcaaaatccaggtctagctcaaaatcccattctagaaggaaaagatcacaatcaaaacacaggagtagatcccataatagatcacgttcaagacagaaagacagacgtagatctaagagcccacataaaaaacgctctaaatcaagggagagacggaagtcaaggagtcgttcgcattcacgggaaaggcgtaggaggaggagcaggagttcttccagatcgccaagaacatcaaaaaccataaaaaggaaatcttctagatctccgtcccccaggagcagaaataagaaggataaaaagagagaaaaagaaagggaccacatcagtgaaagaagagagagagaacgttcaacgtctatgagaaagagttctaatgatagagatgggaaggagaagttggagaagaacagtacttcacttaaagagaaagagcacaataaagaaccagattcaagtgtgagcaaagaagtagatgacaaggatgcaccaaggactgaggaaaacaaaatacagcacaatgggaattgtcagctgaatgaagaaaacctctctaccaaaacagaagcagtataggaccgacaagtgtacctctgcactcaatgctggaatcaaatcc

SEQ ID NO: 775 - DNA, 1952 bp, Homo sapiens
aaactaaagcacccgacgacttagttgctccggtcgtgaagaaaccacacatctattatggaagtttggaagagaaggagagggagcgtctggccaaaggagagtctgggattttggggaaagacggacttaaagcagggatcgaagctggaaatattaatataacctctggagaagtgtttgaaattgaagagcatatcagcgagcgacaggcagaagtattggctgagtttgagagaaggaagcgagcccggcagatcaatgtttccacagatgactcagaggtcaaagcttgccttagagccttgggggaacccatcacactttttggagagggtcctgctgaaagaagagaaaggttaagaaatatcctctcagttgtcggtactgatgccttgaaaaagaccaaaaaggatgatgagaagtctaaaaagtccaaagaagaggtagaacatgtctttaacttcacagtataaacatgaaggaaatgaggggataggtctctcgttttctgctttcaatggtttgttttgctgagatgttgggggaaatgtttttgaaggctctaccattcaagaagagttgctggcagtagttttggttcctttgtaagtatgaatggagctaagtgagttttccagtcaggaaagaatcatggcattcctggtataaccatgtagttacatatcatagaaaaaaattcagtagaaagtcctctgcctgatttcatcctattaccgaatgaattcaccttccttctgggcagttaaaatggagaaatgacagttataagaggagtagaatgcttcagatttgacctttctgctcttaatttgcctttcagtatcagcaaacctggtatcatgaaggaccaaatagcttgaaggtggcaagactatggattgctaattattcgttgcccagggcaatgaaacgcttggaagaggcccgactccataaggagattcctgagacaacaaggacctcccagatgcaagagctgcacaagtctctccggtctttgaataatttttgcagtcagattggggatgatcggcctatctcctactgtcactttagtcccaattccaagatgctggccacagcttgttggagtgggctttgcaagctctggtctgttcctgattgcaacctccttcacactcttcgagggcataacacaaatgtaggagcaattgtattccatcccaaatccactgtctccttggacccaaaagatgtcaacctggcctcttgtgcggctgatggctctgtgaagctttggagtctcgacagtgatgaaccagtggcagatattgaaggccatacagtgcgtgtggcgcgggtaatgtggcatccttcaggacgtttcctgggcaccacctgctatgaccgttcatggcgcttatgggatttggaggctcaagaggagatcctgcatcaggaaggccatagcatgggtgtgtatgacattgccttccatcaagatggctctttggctggcactgggggactggatgcatttggtcgagtttgggacctacgcacaggacgttgtatcatgttcttagaaggccacctgaaagaaatctatggaataaatttctcccccaatggctatcacattgcaaccggcagtggtgacaacacctgcaaagtgtgggacctccgacagcggcgttgcgtctacaccatccctgctcatcagaacttagtgactggtgtcaagtttgagcctatccatgggaacttcttgcttactggtgcctatgataacacagccaagatctggacgcacccaggctggtccccgctgaagactctggctggccacgaaggcaaagtgatgggcctagatatttcttccgatgggcagctcatagccacttgctcatatgacaggaccttcaagctgtggatggctgaatagatgacaatgggaaaaggacttg

SEQ ID NO: 776 - DNA, 1665 bp, Homo sapiens
aaactaaagcacccgacgacttagttgctccggtcgtgaagaaaccacacatctattatggaagtttggaagagaaggagagggagcgtctggccaaaggagagtctgggattttggggaaagacggacttaaagcagggatcgaagctggaaatattaatataacctctggagaagtgtttgaaattgaagagcatatcagcgagcgacaggcagaagtattggctgagtttgagagaaggaagcgagcccggcagatcaatgtttccacagatgactcagaggtcaaagcttgccttagagccttgggggaacccatcacactttttggagagggtcctgctgaaagaagagaaaggttaagaaatatcctctcagttgtcggtactgatgccttgaaaaagaccaaaaaggatgatgagaagtctaaaaagtccaaagaagagtatcagcaaacctggtatcatgaaggaccaaatagcttgaaggtggcaagactatggattgctaattattcgttgcccagggcaatgaaacgcttggaagaggcccgactccataaggagattcctgagacaacaaggacctcccagatgcaagagctgcacaagtctctccggtctttgaataatttttgcagtcagattggggatgatcggcctatctcctactgtcactttagtcccaattccaagatgctggccacagcttgttggagtgggctttgcaagctctggtctgttcctgattgcaacctccttcacactcttcgagggcataacacaaatgtaggagcaattgtattccatcccaaatccactgtctccttggacccaaaagatgtcaacctggcctcttgtgcggctgatggctctgtgaagctttggagtctcgacagtgatgaaccagtggcagatattgaaggccatacagtgcgtgtggcgcgggtaatgtggcatccttcaggacgtttcctgggcaccacctgctatgaccgttcatggcgcttatgggatttggaggctcaagaggagatcctgcatcaggaaggccatagcatgggtgtgtatgacattgccttccatcaagatggctctttggctggcactgggtaaggcttctcccatgtagtcaggggcagttcagtactctcacctcttacctatacctgcttccacagagaactggattcaaagtgttcatttctaaattattttctcaggggactggatgcatttggtcgagtttgggacctacgcacaggacgttgtatcatgttcttagaaggccacctgaaagaaatctatggaataaatttctcccccaatggctatcacattgcaaccggcagtggtgacaacacctgcaaagtgtgggacctccgacagcggcgttgcgtctacaccatccctgctcatcagaacttagtgactggtgtcaagtttgagcctatccatgggaacttcttgcttactggtgcctatgataacacagccaagatctggacgcacccaggctggtccccgctgaagactctggctggccacgaaggcaaagtgatgggcctagatatttcttccgatgggcagctcatagccacttgctcatatgacaggaccttcaagctgtggatggctgaatagatgacaatgggaaaaggacttg

SEQ ID NO: 777 - DNA, 1204 bp, Homo sapiens
gcaccgcatctacgagtatgtggagtcccggatgtccttcatcgcacccaacctgtccatcattatcggggcatccacggccgccaagatcatgggtgtggccggcggcctgaccaacctctccaagatgcccgcctgcaacatcatgctgctcggggcccagcgcaagacgctgtcgggcttctcgtctacctcagtgctgccccacaccggctacatctaccacagtgacatcgtgcagtccctgccaccggatctgcggcggaaagcggcccggctggtggccgccaagtgcacactggcagcccgtgtggacagtttccacgagagcacagaagggaaggtgggctacgaactgaaggatgagatcgagcgcaaattcgacaagtggcaggagccgccgcctgtgaagcaggtgaagccgctgcctgcgcccctggatggacagcggaagaagcgaggcggccgcaggtaccgcaagatgaaggagcggctggggctgacggagatccggaagcaggccaaccgtatgagcttcggagagatcgaggaggacgcctaccaggaggacctgggattcagcctgggccacctgggcaagtcgggcagtgggcgtgtgcggcagacacaggtaaacgaggccaccaaggccaggatctccaagacgctgcaggtatgggccagacccaggtggggctggggaccgagggacacaaggtggggggagcccagatcgcagcctccctgtcctccccacagcggaccctgcagaagcagagcgtcgtatatggcgggaagtccaccatccgcgaccgctcctcgggcacggcctccagcgtggccttcaccccactccagggcctggagattgtgaacccacaggcggcagagaagaaggtggctgaggccaaccagaagtatttctccagcatggctgagttcctcaaggtcaagggcgagaagagtggccttatgtccacctgaatgactgcgtgtgtccaaggtggcttcccactgaagggacacagaggtccagtccttctgaagggctaggatcgggttctggcagggagaacctgccctgccactggccccattgctgggactgcccagggaggaggccttggaagagtccggcctggcctcccccaggaccgagatcaccgcccagtatgggctagagcaggttttcatcatgccttgt

SEQ ID NO: 778 - DNA, 1308 bp, Homo sapiens
gcaccgcatctacgagtatgtggagtcccggatgtccttcatcgcacccaacctgtccatcattatcggggcatccacggccgccaagatcatgggtgtggccggcggcctgaccaacctctccaagatgcccgcctgcaacatcatgctgctcggggcccagcgcaagacgctgtcgggcttctcgtctacctcagtgctgccccacaccggctacatctaccacagtgacatcgtgcagtccctgccaccggatctgcggcggaaagcggcccggctggtggccgccaagtgcacactggcagcccgtgtggacagtttccacgagagcacagaagggaaggtgggctacgaactgaaggatgagatcgagcgcaaattcgacaagtggcaggagccgccgcctgtgaagcaggtgaagccgctgcctgcgcccctggatggacagcggaagaagcgaggcggccgcaggtgaggggccctgggggtccggtaggcatgggggtcatggaggggagaagccggcgtcctcctcccagccgactccctggcgccgcccacccacccgtccccaggtaccgcaagatgaaggagcggctggggctgacggagatccggaagcaggccaaccgtatgagcttcggagagatcgaggaggacgcctaccaggaggacctgggattcagcctgggccacctgggcaagtcgggcagtgggcgtgtgcggcagacacaggtaaacgaggccaccaaggccaggatctccaagacgctgcaggtatgggccagacccaggtggggctggggaccgagggacacaaggtggggggagcccagatcgcagcctccctgtcctccccacagcggaccctgcagaagcagagcgtcgtatatggcgggaagtccaccatccgcgaccgctcctcgggcacggcctccagcgtggccttcaccccactccagggcctggagattgtgaacccacaggcggcagagaagaaggtggctgaggccaaccagaagtatttctccagcatggctgagttcctcaaggtcaagggcgagaagagtggccttatgtccacctgaatgactgcgtgtgtccaaggtggcttcccactgaagggacacagaggtccagtccttctgaagggctaggatcgggttctggcagggagaacctgccctgccactggccccattgctgggactgcccagggaggaggccttggaagagtccggcctggcctcccccaggaccgagatcaccgcccagtatgggctagagcaggttttcatcatgccttgt

SEQ ID NO: 779 - DNA, 1106 bp, Homo sapiens
ccccctaaatctggaaaaatgaacatgaacatccttcaccaggaagagctcatcgctcagaagaaacgggaaattgaagccaaaatggaacagaaagccaagcagaatcaggtggccagccctcagcccccacatcctggcgaaatcacaaatgcacacaactcttcctgcatttccaacaagtttgccaacgatggtagcttcttgcagcagtttctgaagttgcagaaggcacagaccagcacagacgccccgaccagtgcgcccagcgcccctcccagcacacccacccccagcgctgggaagaggtccctgctcatcagcaggcggacaggcctggggctggccagcctgccgggccctgtgaagagctactcccacgccaagcagctgcccgtggcgcaccgcccgagtgtcttccagtcccctgacgaggacgaggaggaggactatgagcagtggctggagatcaaagagagagtgtgcctattgactgtggggtgtgtgagttgaaccccagtactgacagcctccttaaagtttcacccccagagggagccgagactcggaaagtgatagagaaattggcccgctttgtggcagaaggaggccccgagttagaaaaagtagctatggaggactacaaggataacccagcatttgcatttttgcacgataagaatagcagggaattcctctactacaggaagaaggtggctgagataagaaaggaagcacagaagtcgcaggcagcctctcagaaagtttcacccccagaggacgaagaggtcaagaaccttgcagaaaagttggccaggttcatagcggacgggggtcccgaggtggaaaccattgccctccagaacaaccgtgagaaccaggcattcagctttctgtatgagcccaatagccaagggtacaagtactaccgacagaagctggaggagttccggaaagccaaggccagctccacaggcagcttcacagcacctgatcccggcctgaagcgcaagtcccctcctgaggccctgtcagggtccttacccccagccaccacctgccccgcctcgtccacgcctgcgcccactatcatccctgctccagct

SEQ ID NO: 780 - DNA, 1035 bp, Homo sapiens
caaggacattgaggacgtgttctacaaatacggcgctatccgcgacatcgacctcaagaatcgccgcgggggaccgcccttcgccttcgttgagttcgaggacccgcgagacgcggaagacgcggtgtatggtcgcgacggctatgattacgatgggtaccgtctgcgggtggagtttcctcgaagcggccgtggaacaggccgaggcggcggcgggggtggaggtggcggagctccccgaggtcgctatggccccccatccaggcggtctgaaaacagagtggttgtctctggactgcctccaagtggaagttggcaggatttaaaggatcacatgcgtgaagcaggtgatgtatgttatgctgatgtttaccgagatggcactggtgtcgtggagtttgtacggaaagaagatatgacctatgcagttcgaaaactggataacactaagtttagatctcatgaggtaggttatacacgtattcttttctttgaccagaattggatacagtggtcttaacagtggaatttcaaggtaaggattcaggcaaggttgtccaagtaaattgccagatttctggttttagttacattgtattcattcagcatgtctgaagatagatgaaagcttagatctttcaatggaaagttctgtctatccaatagggagaaactgcctacatccgggttaaagttgatgggcccagaagtccaagttatggaagatctcgatctcgaagccgtagtcgtagcagaagccgtagcagaagcaacagcaggagtcgcagttactccccaaggagaagcagaggatcaccacgctattctccccgtcatagcagatctcgctctcgtacataagatgattggtgacactttttgtagaacccatgttgtatacagttttcctttattcagtacaatcttttcattttttaattcaaactgttttgttcagaatgggctaaagtgttgaattgcattcttgtaatatccccttgctcctaacatctacattcccttcgtgtctttgat

SEQ ID NO: 781 - DNA, 872 bp, Homo sapiens
caaggacattgaggacgtgttctacaaatacggcgctatccgcgacatcgacctcaagaatcgccgcgggggaccgcccttcgccttcgttgagttcgaggacccgcggtgaggcggcatggggcttgcagccttgaggaaatagagacgcggaagacgcggtgtatggtcgcgacggctatgattacgatgggtaccgtctgcgggtggagtttcctcgaagcggccgtggaacaggccgaggcggcggcgggggtggaggtggcggagctccccgaggtcgctatggccccccatccaggcggtctgaaaacagagtggttgtctctggactgcctccaagtggaagttggcaggatttaaaggatcacatgcgtgaagcaggtgatgtatgttatgctgatgtttaccgagatggcactggtgtcgtggagtttgtacggaaagaagatatgacctatgcagttcgaaaactggataacactaagtttagatctcatgagggagaaactgcctacatccgggttaaagttgatgggcccagaagtccaagttatggaagatctcgatctcgaagccgtagtcgtagcagaagccgtagcagaagcaacagcaggagtcgcagttactccccaaggagaagcagaggatcaccacgctattctccccgtcatagcagatctcgctctcgtacataagatgattggtgacactttttgtagaacccatgttgtatacagttttcctttattcagtacaatcttttcattttttaattcaaactgttttgttcagaatgggctaaagtgttgaattgcattcttgtaatatccccttgctcctaacatctacattcccttcgtgtctttgat

SEQ ID NO: 782 - DNA, 883 bp, Homo sapiens
agcaggaagaggagattctgggatctgatgatgatgagcaagaagatcctaatgattattgtaaaggaggttatcatcttgtgaaaattggagatctattcaatgggagataccatgtgatccgaaagttaggctggggacacttttcaacagtatggttatcatgggatattcaggggaagaaatttgtggcaatgaaagtagttaaaagtgctgaacattacactgaaacagcactagatgaaatccggttgctgaagtcagttcgcaattcagaccctaatgatccaaatagagaaatggttgttcaactactagatgactttaaaatatcaggagttaatggaacacatatctgcatggtatttgaagttttggggcatcatctgctcaagtggatcatcaaatccaattatcaggggcttccactgccttgtgtcaaaaaaattattcagcaagtgttacagggtcttgattatttacataccaagtgccgtatcatccacactgacattaaaccagagaacatcttattgtcagtgaatgagcagtacattcggaggctggctgcagaagcaacagaatggcagcgatctggagctcctccgccttccggatctgcagtcagtactgctccccagcctaaaccaaagagtcaagtaccattggccaggatcaaacgcttatggaacgtgatacagagggtggtgcagcagaaattaattgcaatggagtgattgaagtcattaattatactcagaacagtaataatgaaacattgagacataaagaggatctacataatgctaatgactgtgatgtccaaaatttgaatcaggaatctagtttcctaagctcccaaaatggagacagcagcacatct

SEQ ID NO: 783 - DNA, 1150 bp, Homo sapiens
aaatgcatcgtgattcctgtccattggactgtaaggtttatgtaggcaatcttggaaacaatggcaacaagacggaattggaacgggcttttggctactatggaccactccgaagtgtgtgggttgctagaaacccacccggctttgcttttgttgaatttgaagatccccgagatgcagctgatgcagtccgagagctagatggaagaacactatgtggctgccgtgtaagagtggaactgtcgaatggtgaaaaaagaagtagaaatcgtggcccacctccctcttggggtcgtcgccctcgagatgattatcgtaggaggagtcctccacctcgtcgcagagtcaccatcatgtctcttctcaccaccctctgaatctgcattagccagtcaactagccctttcagcgtcatgtgaccagcgcgccccattcagcttggctggtgtcgtttcacatgacccaggctggccagtcgtcaggttgcaccgccctttggttcccgagcatgctgttttctctcagccttctctccaaccttaaccaaatcggcagcagccacctcgaccgcccacacattcctggccaatcagctcagctgtttatttaccaaatgtcttcacaacaactacagcagcagccttcggctaacaaaaaagcaggaaaaatccacaacacccccttcgccaaccaactaaatccaacgcaacatctggcaaaaccttttcagcaaattcttcctggccgtcagtccggcagcctcacctcaccatttctagcttgttgaaacccaaaactaatctccaagaaggagaagcttctctcgcagccggagcaggtccctttctagagataggagaagagagagatcgctgtctcgggagagaaatcacaagccgtcccgatccttctctaggtctcgtagtcgatctaggtcaaatgaaaggaaatagaagacagtttgcaagagaagtggtgtacaggaaattacttcatttgacaggagtatgtacagaaaattcaagttttgtttgagacttcataagcttggtgcatttttaagatgttttagctgttcaaatctgtttgtctcttgaaacagtgacacaaaggtgtaattctctatggtttgaaatggatcatacgaggc

(19) United States
(12) Patent Application Publication
(10) Pub. No.: US 2009/0176724 A1
(43) Pub. Date: Jul. 9, 2009

(54) Methods and Compositions for the Diagnosis, Prognosis and Treatment of Cancer

(76) Inventors: Daiwei Shen, South Pasadena, CA (US); Toomas Neuman, Mountain View, CA (US); Kaia Palm, Tallinn (EE)

Correspondence Address: DORSEY & WHITNEY LLP;INTELLECTUAL PROPERTY DEPARTMENT, 370 SEVENTEENTH STREET, SUITE 4700, DENVER, CO, 80202-5647, US

(21) Appl. No.: 11/571,585

(22) PCT Filed: Jun. 30, 2005
(86) PCT No.: PCT/US05/23708

§ 371 Date: Dec. 1, 2008

Related U.S. Application Data
(60) Provisional application No. 60/584,784, filed on Jun. 30, 2004.

(51) Int. Cl. A61K 31/7052; G01N 33/574; C40B 30/00; C12Q 1/68; C07K 14/00; C40B 40/10; A61P 35/00; C07K 16/00; C07H 21/00; G01N 33/53; C40B 30/04

(52) U.S. Cl. 514/44; 436/64; 506/7; 435/6; 436/501; 506/9; 536/23.5; 530/350; 530/387.9; 506/18

(57) ABSTRACT

The invention is relates to splice variants of basal transcription factors and other transcriptional modulators, the use of expression analyses of the same as a diagnostic and prognostic tool, and the targeting of such splice variants for therapeutic purposes, particularly in relation to the treatment of cancer.

### STATEMENT OF RELATEDNESS

This application claims the benefit of application Ser. No. 60/584,784, filed Jun. 30, 2004, which is expressly incorporated herein in its entirety by reference.

### FIELD

The present disclosure relates to the expression of transcription modulator splice variants, more particularly to the expression of splice variants of basal transcription factors, and to the early diagnosis, prognosis, and treatment of cancer. The present disclosure further relates to the molecular characterization of cancer and the description of cancer subtypes, as well as the optimization of cancer treatment. The present disclosure further relates to cancer treatment methods and therapeutic agents.

### BACKGROUND

The early and accurate detection of cancer, and the precise characterization of tumor cells are highly desirable for effective cancer treatment. However, many current diagnostic methods, such as those involving imaging and the analysis of biochemical markers, do not reliably provide for early and accurate diagnosis.

A number of studies examining the molecular characteristics of various cancers have been reported. Oligonucleotide and cDNA micro-arrays (Bhattacharjee et al., Proc. Natl. Acad. Sci. USA, 98(24):13790-13795 (2001), Garber et al., Proc. Natl. Acad. Sci. USA 98(24):13784-13789 (2001), Virtanen et al., Proc. Natl. Acad. Sci. USA, 99(19):12357-12362 (2002)), as well as the serial analysis of gene expression (Nacht et al., Proc. Natl. Acad. Sci. USA, 98(26):15203-15208 (2001)) have been used to molecularly characterize different cancer types. In addition, the expression of particular markers has been associated with prognosis for particular cancers (Beer et al., Nature Medicine, 8(8):816-824 (2002), Volm et al., Clinical Cancer Res., 8:1843-1848 (2002), Wigle et al., Cancer Res., 62:3005-3008 (2002)). Tumor cells have also been shown to express splice variant mRNAs that are not present in normal cells of the same cell type. A genome-wide computational screen using human expressed sequence tags identified more than 25,000 alternatively spliced transcripts, of which 845 were significantly associated with cancer (Wang et al., Cancer Research 63:655-657 (2003)).

Differences between the gene expression profiles of cancer cells and normal cells, and the presence of cancer cell markers, stem in part from differences in patterns of transcriptional activity between cancer and normal cells. It is well known that a number of identified oncogenes encode transcription factors. In addition, it has been reported that some tumor cells aberrantly express transcriptional modulators that are normally expressed during development (Palm et al., Brain Res. Mol. Brain. Res. 72(1):30-39 (1999), Lee et al., J. Mol. Neurosci., 15(3):205-214 (2000), Lawinger et al., Nat. Med., 6(7):826-831 (2000), Coulson et al., Cancer Res., 60(7):1840-1844 (2000), Gure et al., Proc. Natl. Acad. Sc. USA., 97(8):4198-203. (2000)). WO 02/40716 in particular discloses the expression profiles of a number of transcription factors in a variety of cancers, and describes tumor subtypes that express subsets of transcription factors.

Studies examining the immunoreactivity of blood sera from cancer patients have also been reported. Serological analysis of expression cDNA libraries has been used to identify tumor antigens, among which developmentally regulated transcription factors have been found (Gure et al., 2000). Additionally, WO 02/40716 discloses the use of peptides derived from developmentally regulated transcription factors to generate an anti-transcription-factor autoantibody profile detailing the aberrant expression of the transcription factors in tumor cells. However, because these transcription factors are not tumor-specific and are potentially exposed to the immune system prior to the onset of cancer, the use of immunoreactivity against such transcription factors to diagnose cancer may be hindered by the occurrence of false positive results.

Improvements in diagnostic and prognostic methods have come from the use cancer-associated transcription modulator splice variants, and autoantibodies recognizing the same, as early markers of cancer. The expression profiles of a plurality of transcription modulator splice variants that are tumor-specific or tumor-enriched (“tumor-specific/enriched”) and their correlation with numerous cancer types and subtypes has been described (PCT/US03/41253, expressly incorporated herein in its entirety by reference). Further, the utility of expression profiles of such transcription modulator splice variants as a very highly accurate diagnostic indicator for the early detection of cancer has been established. Additionally, the utility of expression profiles of an appropriate set of such transcription modulator splice variants as a very highly accurate diagnostic indicator for a variety of cancer types has been established.

Devices for identifying differentially spliced gene products have also been described previously (U.S. Pat. No. 6,881,571; U.S. Pub. 2004/0191828). Additionally, methods for remotely detecting cancer using nucleic acids prepared from blood cells and involving the hybridization thereof to splicing forms of nucleic acids associated with cancer have been described (U.S. Pat. No. 6,372,432). However, these devices and methods have not been directed to the detection of transcription modulators and splice variants thereof in cancer cells in particular. As such, they may not be capable of detecting the earliest molecular alterations associated with cell transformation, and may not provide the mechanistic insight highly desired for the design of cancer therapeutics.

### SUMMARY OF THE INVENTION

The number and nature of biomarkers that are used in a diagnostic or prognostic assay controls the accuracy of the diagnostic or prognostic determination. While the expression of transcription factors in a variety of cancer types has been previously reported, and the use of such expression profiles as a diagnostic tool has been disclosed in WO 02/40716, the present methods are distinguished in one respect by their reliance on the expression profiles of tumor-enriched or tumor-specific splice variants of transcription modulators, which are more specific to cancer and, in many tumor types, more highly expressed than their wildtype counterparts. The present disclosure thus provides diagnostics that are both more sensitive and more accurate than those disclosed in WO 02/40716.

The use of expression profiles of transcription modulator splice variants in diagnostic and prognostic methods has been previously disclosed by the present inventors (PCT/US03/41253). However, the present invention stems in large part from the surprising recent finding that a large number of splice variants of basal transcription factors are present in significant amounts in a wide variety of cancers. Previous studies did not reveal the predominance of this particular class of transcription modulator splice variants in cancer cells. This, combined with the low expression level of basal transcription factors relative to other transcription modulators suggested that basal transcription factor splice variants might not be a preferred class for use in diagnostic and prognostic assays. However, the ubiquitous expression of basal transcription factors and their intimate association with the regulation of gene transcription by RNA Polymerase II, combined with the present identification of large numbers of aberrant basal transcription factor splice variants associated with a wide variety of cancer types now makes the basal transcription factor class of splice variants a highly preferred class for use in diagnostic and prognostic assays.

In addition to establishing the significance of basal transcription factor splice variants, the present invention discloses a large number of splice variants in addition to those disclosed in PCT/US03/41253, the expression characteristics of which may be used to improve the accuracy of diagnostic and prognostic methods, as well as increase the resolution of cancer subtypes at the molecular level. Further, the presently disclosed transcription modulator splice variants represent novel targets for therapeutic agents, as described herein.

Accordingly, disclosed herein are methods and compositions for diagnosing cancer. Further disclosed herein are methods and compositions for diagnosing cancer subtypes. Further disclosed herein are methods and compositions for determining the prognosis of a patient having cancer. Further disclosed herein are methods and compositions for the treatment of cancer. The diagnostic methods provided herein generally comprise determining the expression of a plurality of tumor-specific/enriched splice variants of transcription modulators, more particularly a plurality of tumor-specific/enriched splice variants of basal transcription factors. Typically, the expression of at least two, more preferably at least 5, still more preferably at least 10, and often at least 15, 25 or 50 splice variants of basal transcription factors is determined, though generally the expression of not more than about 5000, more preferably less than about 1000 or 500, and still more preferably less than about 250 or 100 such splice variants is determined in the subject methods. In one embodiment, the methods further comprise determining the expression of one or more splice variants of non-basal transcription factors to increase the accuracy of the method and/or the resolution of cancer subtypes. Preferably, the expression of at least one, more preferably at least two, more preferably at least 10, and often more than 15, 50, or 100 splice variants of non-basal transcription factors will be determined. Typically, the expression of less than 5000, and more often less than 1000, and most often less than 500 of such splice variants of non-basal transcription factors will be determined.

In a preferred embodiment, the expression of at least one splice variant of each of a plurality of basal transcription factors is determined. In a preferred embodiment, the expression of at least one splice variant of between at least two and about 1000, more preferably between at least two and about 500, more preferably between at least two and about 250, more preferably between at least two and about 150, more preferably between at least two and about 100, more preferably between at least two and about 75, more preferably between at least two and about 50, more preferably between at least two and about 25, more preferably between at least two and about 10 basal transcription factors is determined, wherein expression of each of the basal transcription factor splice variants is indicative of cancer.

In another preferred embodiment, the expression of a plurality of splice variants of a basal transcription factor is determined. In a preferred embodiment, the expression of between at least two and about 10 or 20, more preferably between at least two and about 5 splice variants of a basal transcription factor is determined, wherein expression of each of the basal transcription factor splice variants is indicative of cancer.

In a preferred embodiment, one or more of the basal transcription factor splice variants is derived from a gene selected from group of gene families consisting of TAF, SMARC, HDAC, MED12, NCOA, GTF, THRAP, HMG, OGDL, BRF, and BAF.

In a further preferred embodiment, one or more of the basal transcription factor splice variants is derived from a gene selected from the group of gene families consisting of TAF2, TAF4, TAF6L, TAF7L, TAF8, TAF10, TAF15, SMARCA1, SMARCA2, SMARCA4, SMARCA5 SMARCB1, SMARCC2, SMARCD3, NCOA2, NCOA3, NCOA4, NCOA6, NCOA7, BRF1, GTF3C, GTF2F, MED12, THRAP4, THRAP3, HMG20, OGHDL, HDAC5, AND BAF250.

In a preferred embodiment, the methods further comprise determining the expression of at least one splice variant of each of a plurality of transcription modulators which are not basal transcription factors. In a preferred embodiment, the expression of at least one splice variant of between at least two and about 1000, more preferably between at least two and about 500, more preferably between at least two and about 250, more preferably between at least two and about 150, more preferably between at least two and about 100, more preferably between at least two and about 75, more preferably between at least two and about 50, more preferably between at least two and about 25, more preferably between at least two and about 10 such transcription modulators is determined, wherein expression of each such splice variant is indicative of cancer.

In another preferred embodiment, the methods further comprise determining the expression of a plurality of splice variants of a transcription modulator which is not a basal transcription factor. In a preferred embodiment, the expression of between at least two and about 10 or 20, more preferably between at least two and about 5 such splice variants is determined, wherein expression of each of the splice variants is indicative of cancer.

In another preferred embodiment, the methods further comprise determining the expression of one or more splice variants which are not transcription factors. In another preferred embodiment, the methods further comprise determining the expression of one or more such splice variants. It will be appreciated that splice variants of transcription factors, and of basal transcription factors in particular, are preferred therapeutic targets, and knowledge of their expression in disease cells is, accordingly, highly desired. However, splice variants of non-transcription factors and non-transcription modulators are also present in cancer cells and are diagnostically useful in combination with transcription factor splice variants for increased diagnostic accuracy and for the identification of molecular subtypes of cancer, which reflect the varied regulatory mechanisms between cancer cells.

The expression of a plurality of basal transcription factor splice variants and splice variants of other factors may be determined simultaneously or sequentially.

Though the splice variants provided herein are indicative of cancer, each splice variant is not necessarily expressed in all cancers, all tumor cell types, or all patients having a particular type of cancer (e.g., prostate cancer; small cell lung cancer). Further, in some embodiments, the set of transcription modulator splice variants for which expression is determined in a diagnostic assay will include one or more that are determined not to be expressed (i.e., in addition to the plurality that are determined to be expressed). As disclosed herein, it is the overall expression pattern, i.e., the combined determinations of the expression of a plurality of splice variants, not individual splice variants, that provides for the highly accurate diagnosis of cancer. Thus, negative expression results are obtained for individual splice variants in some diagnostic and prognostic assays disclosed herein, yet the assay results are indicative of cancer or a particular prognosis.

It will be apparent to one of skill in the art that the information gleaned from the determination of the expression of a plurality of basal transcription factor splice variants, and optionally one or more additional splice variants is, as exemplified herein, not simply additive. Rather, the combinatorial analysis of tumor-enriched/specific splice variant expression disclosed herein reveals molecular subtypes of cancer, in which the expression of a number of such splice variants is linked. Thus, the splice variants presently disclosed in addition to those disclosed in PCT/US03/41253 provide for more accurate diagnostic determinations than those disclosed in PCT/US03/41253, as well as for the enhanced resolution and identification of novel molecular subtypes of cancer.

The present methods and compositions thus satisfy the need for highly accurate diagnostic and prognostic assays, and provide for the precise characterization of tumor cells and the identification of cancer subtypes. Importantly, the present methods and compositions provide by way of the analysis of transcription factor splice variants, particularly basal transcription modulator splice variants, the mechanistic insight highly desired for the design of cancer therapeutics.

In a preferred embodiment disclosed herein are methods for diagnosing cancer subtypes. The methods generally comprise determining the expression of a plurality of tumor-specific/enriched splice variants of basal transcription factors. In a preferred embodiment, the methods comprise determining the expression of at least one splice variant of a plurality of basal transcription factors, wherein the presence or absence of expression of each splice variant is indicative of a cancer subtype. In another preferred embodiment, the methods comprise determining the expression of a plurality of splice variants of a basal transcription factor, wherein the presence or absence of expression of each splice variant is indicative of a cancer subtype. In a preferred embodiment, the cancer subtype is characterized by its metastatic potential. In another embodiment, the cancer subtype is characterized by its refractory behavior, particularly its non-responsiveness to a therapeutic agent. In another preferred embodiment, the cancer subtype is characterized by its invasive activity.

In a preferred embodiment, one or more of the basal transcription factor splice variants is derived from a gene selected from group of gene families consisting of TAF, SMARC, HDAC, MED12, NCOA, GTF, THRAP, HMG, OGDL, BRF, and BAF.

In a further preferred embodiment, one or more of the basal transcription factor splice variants is derived from a gene selected from the group of gene families consisting of TAF2, TAF4, TAF6L, TAF7L, TAF8, TAF10, TAF15, SMARCA1, SMARCA2, SMARCA4, SMARCA5 SMARCB1, SMARCC2, SMARCD3, NCOA2, NCOA3, NCOA4, NCOA6, NCOA7, BRF1, GTF3C, GTF2F, MED12, THRAP4, THRAP3, HMG20, OGHDL, HDAC5, AND BAF250.

In some embodiments, the methods further comprise determining the expression of a plurality of tumor-specific/enriched splice variants of non-basal transcription factors. In a preferred embodiment, the methods comprise determining the expression of at least one splice variant of a plurality of non-basal transcription factors, wherein the presence or absence of expression of each splice variant is indicative of a cancer subtype. In another preferred embodiment, the methods comprise determining the expression of a plurality of splice variants of a non-basal transcription factor, wherein the presence or absence of expression of each splice variant is indicative of a cancer subtype. In a preferred embodiment, the cancer subtype is characterized by its metastatic potential.

In another embodiment, the cancer subtype is characterized by its refractory behavior, particularly its non-responsiveness to a therapeutic agent. In another preferred embodiment, the cancer subtype is characterized by its invasive activity.

In a preferred embodiment, the methods further comprise determining the expression of additional splice variants which are useful for diagnosing cancer and cancer subtypes. Preferred splice variants for use in the present methods include those disclosed herein. In one embodiment, the expression of markers such as integrins, receptors for extracellular signals including receptor tyrosine kinases, non-receptor tyrosine kinases, matrix metalloproteinases, and other molecules known to have a role in signal transduction, cell proliferation, cell motility, cell adhesion, or cell survival are also determined.

In another preferred embodiment disclosed herein are methods for determining cancer prognosis, which comprise diagnosing a cancer subtype as disclosed herein. In a preferred embodiment, the methods further comprise determining the expression of additional prognostic indicators known in the art.

Determining splice variant expression may involve determining mRNA or protein expression, which may be done using any of the large number of methods known in the art. Alternatively, determining splice variant expression may involve determining the presence of autoantibodies that recognize the splice variant.

A preferred method for determining expression involves the use of RT-PCR to determine the expression of splice variant mRNAs. The primers used to detect splice variant mRNAs preferably hybridize to sequences flanking junction sites of deletionsor to sequences flanking or in inserted sequences. Preferred primers for determining the expression of splice variant mRNAs include those disclosed herein. Additionally preferred primers are disclosed in PCT/US03/41253. Additionally, it will be appreciated that primers may be designed based on the sequence of splice variant mRNAs using routine methods.

Another preferred method for determining expression involves the use oligonucleotide probes to determine the expression of splice variant mRNAs. In a particularly preferred embodiment, the oligonucleotide probes are on an array. Another preferred method for determining expression involves the use of peptides that are capable of detecting auto-antibodies that specifically bind to transcription modulator splice variants. The peptides preferably do not specifically bind to autoantibodies that specifically bind to wildtype isoforms of the transcription modulators. In a particularly preferred embodiment, the peptides are on an array.

Importantly, the methods provided herein provide for distinguishing the expression of splice variants of from the expression of “wildtype” counterpart isoforms. As disclosed herein, many tumor-specific/enriched splice variants of transcription modulators have wildtype counterparts that are expressed in non-tumor cells. Consequently, distinguishing splice variant from wildtype isoform expression contributes significantly to the accuracy of the diagnostic methods disclosed herein.

Preferred splice variants are those associated with cancer, particularly cancer selected from the group consisting of lung cancer (e.g., small cell lung cancer, non-small cell lung cancer), gastrointestinal cancer (e.g., colorectal cancer, stomach cancer, liver cancer, pancreatic cancer, and cancers of other regions of gastrointestinal tract), breast cancer, prostate cancer, skin cancer (e.g., basal cell carcinoma, melanoma), sarcoma, endocrine cancer (e.g., carcinoids, insulinoma, cancer of thyroid gland), neural cancers (e.g., neuroblastoma, glioblastoma, medulloblastoma, retinoblastoma), bladder cancer, cervical cancer, renal cancer, hematopoietic cancers (e.g., lymphoma, leukemia). Also preferred are splice variants for which the presence or absence of expression is indicative of a cancer subtype, particularly a subtype within a cancer selected from the group consisting of lung cancer (e.g., small cell lung cancer, non-small cell lung cancer), gastrointestinal cancer (e.g., colorectal cancer, stomach cancer, liver cancer, pancreatic cancer, and cancers of other regions of gastrointestinal tract), breast cancer, prostate cancer, skin cancer (e.g., basal cell carcinoma, melanoma), sarcoma, endocrine cancer (e.g., carcinoids, insulinoma, cancer of thyroid gland), neural cancers (e.g., neuroblastoma, glioblastoma, medulloblastoma, retinoblastoma), bladder cancer, cervical cancer, renal cancer, hematopoietic cancers (e.g., lymphoma, leukemia).

Preferred splice variants for use in the presently disclosed methods are basal transcription factor splice variants that are tumor-specific/enriched.

In a preferred embodiment, one or more of the basal transcription factor splice variants is derived from a gene selected from group of gene families consisting of TAF, SMARC, HDAC, MED12, NCOA, GTF, THRAP, HMG, OGDL, BRF, and BAF.

In a further preferred embodiment, one or more of the basal transcription factor splice variants is derived from a gene selected from the group of gene families consisting of TAF2, TAF4, TAF6L, TAF7L, TAF8, TAF10, TAF15, SMARCA1, SMARCA2, SMARCA4, SMARCA5 SMARCB1, SMARCC2, SMARCD3, NCOA2, NCOA3, NCOA4, NCOA6, NCOA7, BRF1, GTF3C, GTF2F, MED12, THRAP4, THRAP3, HMG20, OGHDL, HDAC5, AND BAF250.

Also preferred in the present invention are combinations of basal transcription factor splice variants provided herein with non-basal transcription factors similarly described herein. Also preferred are combinations including splice variants of NRSF, MDM2, TSG, RREB, ZNF207, TTF-1, GTFIIIA, HES-6, HRY, Msx2, Neu, NeuroD1, Mash-1, and Irx2 which are tumor-specific/enriched, as disclosed in PCT/US03/41253.

Preferred peptides for use in the detection of autoantibodies that recognize tumor-specific/enriched splice variants are those that bind basal transcription factor splice variants and do not specifically bind to autoantibodies that specifically bind to wildtype isoforms of the basal transcription factors.

Preferred peptides include peptides corresponding to amino acid sequences present in transcription modulator splice variants which are not present in wildtype counterparts thereof.

Preferably, where the splice variant disclosed includes a novel amino acid sequence (with respect to its wildtype counterpart), an autoantibody-recognizing peptide corresponds to a region of the splice variant including the novel amino acid sequence, or a portion thereof.

Preferably, where the splice variant includes an in-frame deletion of amino acids present in its wildtype counterpart, an autoantibody-recognizing peptide corresponds to a region of the splice variant including the junction site at which the deletion occurred.

Also preferred are combinations of the peptides described above with those disclosed in PCT/US03/41253.

In another preferred embodiment disclosed herein are peptide arrays, which arrays comprise a plurality of peptides derived from tumor-specific/enriched transcription modulator splice variants, wherein the peptides specifically bind to autoantibodies which are characterized by their ability to specifically bind to transcription modulator splice variants that are tumor-specific/enriched. Moreover, the peptides are splice-variant specific in that they do not bind to autoantibodies that specifically bind to wildtype isoforms of the transcription modulators. Moreover, a plurality of the peptides on such arrays are specific for basal transcription factor splice variants. In a preferred embodiment, one or more of the basal transcription factor splice variants is derived from a gene selected from group of gene families consisting of TAF, SMARC, HDAC, MED12, NCOA, GTF, THRAP, HMG, OGDL, BRF, and BAF. In a further preferred embodiment, one or more of the basal transcription factor splice variants is derived from a gene selected from the group of gene families consisting of TAF2, TAF4, TAF6L, TAF7L, TAF8, TAF10, TAF15, SMARCA1, SMARCA2, SMARCA4, SMARCA5 SMARCB1, SMARCC2, SMARCD3, NCOA2, NCOA3, NCOA4, NCOA6, NCOA7, BRF1, GTF3C, GTF2F, MED12, THRAP4, THRAP3, HMG20, OGHDL, HDAC5, AND BAF250. Such arrays find use in cancer diagnosis, and may particularly be used to determine the expression of a plurality of transcription modulator splice variants simultaneously. In a preferred embodiment, such peptide arrays comprise peptides that specifically bind to autoantibodies that specifically bind to splice variants selected from those described herein. In a preferred embodiment, such peptide arrays additionally comprise peptides disclosed in PCT/US03/41253.

In another preferred embodiment disclosed herein are peptide arrays, which arrays consist essentially of a plurality of peptides derived from tumor-specific/enriched transcription modulator splice variants, wherein the peptides specifically bind to autoantibodies which are characterized by their ability to specifically bind to transcription modulator splice variants that are tumor-specific/enriched. Moreover, the peptides are splice-variant specific in that they do not bind to autoantibodies that specifically bind to wildtype isoforms of the transcription modulators. Moreover, a plurality of the peptides on such arrays are specific for autoantibodies that specifically bind basal transcription factor splice variants. In one embodiment, such arrays consist essentially of peptides specific for autoantibodies that specifically bind basal transcription factor splice variants. In a preferred embodiment, one or more of the basal transcription factor splice variants is derived from a gene selected from group of gene families consisting of TAF, SMARC, HDAC, MED12, NCOA, GTF, THRAP, HMG, OGDL, BRF, and BAF. In a further preferred embodiment, one or more of the basal transcription factor splice variants is derived from a gene selected from the group of gene families consisting of TAF2, TAF4, TAF6L, TAF7L, TAF8, TAF10, TAF15, SMARCA1, SMARCA2, SMARCA4, SMARCA5 SMARCB1, SMARCC2, SMARCD3, NCOA2, NCOA3, NCOA4, NCOA6, NCOA7, BRF1, GTF3C, GTF2F, MED12, THRAP4, THRAP3, HMG20, OGHDL, HDAC5, AND BAF250. Such arrays find use in cancer diagnosis, and may particularly be used to determine the expression of a plurality of transcription modulator splice variants simultaneously. In a preferred embodiment, such peptide arrays consist essentially of peptides that specifically bind to autoantibodies that specifically bind to transcription modulator splice variants selected from those described herein. In another preferred embodiment, such peptide arrays consist essentially of peptides that specifically bind to autoantibodies that specifically bind to transcription modulator splice variants selected from those described herein and peptides disclosed in PCT/US03/41253.

Also disclosed herein in a preferred embodiment are oligonucleotide arrays, which arrays comprise a plurality of oligonucleotides derived from the nucleotide sequences of mRNAs encoding tumor-specific/enriched transcription modulator splice variants, and which hybridize under high stringency conditions to such mRNAs or their complements. Moreover, a plurality of the oligonucleotides of such arrays are specific for basal transcription factor splice variants. In a preferred embodiment, one or more of the basal transcription factor splice variants is derived from a gene selected from group of gene families consisting of TAF, SMARC, HDAC, MED12, NCOA, GTF, THRAP, HMG, OGDL, BRF, and BAF. In a further preferred embodiment, one or more of the basal transcription factor splice variants is derived from a gene selected from the group of gene families consisting of TAF2, TAF4, TAF6L, TAF7L, TAF8, TAF10, TAF15, SMARCA1, SMARCA2, SMARCA4, SMARCA5 SMARCB1, SMARCC2, SMARCD3, NCOA2, NCOA3, NCOA4, NCOA6, NCOA7, BRF1, GTF3C, GTF2F, MED12, THRAP4, THRAP3, HMG20, OGHDL, HDAC5, AND BAF250. Such arrays find use in cancer diagnosis, and may particularly be used to determine the expression of a plurality of transcription modulator splice variants simultaneously. In a preferred embodiment, such arrays comprise oligonucleotides that are substantially complementary to mRNAs selected from those described herein. In another preferred embodiment, such arrays comprise oligonucleotides that are substantially complementary to mRNAs selected from those described herein and splice variants of NRSF, MDM2, TSG, RREB, ZNF207, TTF-1, GTFIIIA, HES-6, HRY, Msx2, Neu, NeuroD1, Mash-1, and Irx2 which are tumor-specific/enriched, as disclosed in PCT/US03/41253.

Also disclosed herein in a preferred embodiment are oligonucleotide arrays, which arrays consist essentially of a plurality of oligonucleotides derived from the nucleotide sequences of mRNAs encoding tumor-specific/enriched transcription modulator splice variants, and which hybridize under high stringency conditions to such mRNAs or their complements. Moreover, a plurality of the oligonucleotides of such arrays are specific for basal transcription factor splice variants. In one embodiment, an array consists essentially of a plurality of oligonucleotides specific for basal transcription factor splice variants. In a preferred embodiment, one or more of the basal transcription factor splice variants is derived from a gene selected from group of gene families consisting of TAF, SMARC, HDAC, MED12, NCOA, GTF, THRAP, HMG, OGDL, BRF, and BAF. In a further preferred embodiment, one or more of the basal transcription factor splice variants is derived from a gene selected from the group of gene families consisting of TAF2, TAF4, TAF6L, TAF7L, TAF8, TAF10, TAF15, SMARCA1, SMARCA2, SMARCA4, SMARCA5 SMARCB1, SMARCC2, SMARCD3, NCOA2, NCOA3, NCOA4, NCOA6, NCOA7, BRF1, GTF3C, GTF2F, MED12, THRAP4, THRAP3, HMG20, OGHDL, HDAC5, AND BAF250. Such arrays find use in cancer diagnosis, and may particularly be used to determine the expression of a plurality of transcription modulator splice variants simultaneously. In a preferred embodiment, such arrays consist essentially of oligonucleotides that are substantially complementary to mRNAs selected from those described herein. In another preferred embodiment, such arrays consist essentially of oligonucleotides that are substantially complementary to mRNAs selected from those described herein and splice variants of NRSF, MDM2, TSG, RREB, ZNF207, TTF-1, GTFIIIA, HES-6, HRY, Msx2, Neu, NeuroD1, Mash-1, and Irx2 which are tumor-specific/enriched, as disclosed in PCT/US03/41253.

In one aspect, the invention provides compositions and methods useful for making amplification products that may be used to probe an oligonucleotide array described herein.

Also disclosed herein are methods for the treatment of cancer, and therapeutics useful in the treatment of cancer.

The treatment methods generally comprise determining the expression of a plurality of tumor-specific/enriched transcription modulator splice variants, wherein the expression of each of the transcription modulator splice variants is indicative of cancer and wherein a plurality of the splice variants are basal transcription factor splice variants, and further comprise administering to the patient a bioactive agent capable of inhibiting the activity of one or more of such splice variants determined to be expressed. In a preferred embodiment, the bioactive agent is targeted to a basal transcription factor splice variant. In a preferred embodiment, the methods comprise determining the expression of at least one splice variant of each of a plurality of transcription modulators. In another preferred embodiment, the methods comprise determining the expression of a plurality of splice variants of a transcription modulator. As in the methods described above, expression of tumor-specific/enriched splice variants is distinguished from the expression of corresponding wildtype isoforms of transcription modulators.

In a preferred embodiment, the treatment methods comprise determining the expression of at least one splice variant of between at least two and about 1000, more preferably between at least two and about 500, more preferably between at least two and about 250, more preferably between at least two and about 150, more preferably between at least two and about 100, more preferably between at least two and about 75, more preferably between at least two and about 50, more preferably between at least two and about 25, more preferably between at least two and about 10 transcription modulators, wherein expression of a plurality of basal transcription factor splice variants is determined, and wherein expression of each of the transcription modulator splice variants is indicative of cancer.

In another preferred embodiment, the expression of a plurality of splice variants of a transcription modulator is determined. In a preferred embodiment, the expression of between at least two and about 10, more preferably between at least two and about 5 splice variants of a transcription modulator is determined, wherein the expression of a plurality of basal transcription factor splice variants is determined, and wherein expression of each of the transcription modulator splice variants is indicative of cancer.

In another preferred embodiment, the treatment methods further comprise diagnosing a cancer subtype, which generally comprises determining the expression of a plurality of transcription modulator splice variants, wherein the expression of a plurality of basal transcription factor splice variants is determined, and wherein the presence or absence of expression of each splice variant is indicative of a cancer subtype. In a preferred embodiment, the methods comprise determining the expression of at least one splice variant of a plurality of transcription modulators, wherein the presence or absence of expression of each splice variant is indicative of a cancer subtype, and further comprise administering to the patient a bioactive agent capable of inhibiting the activity of one or more such splice variants determined to be expressed. In another preferred embodiment, the methods comprise determining the expression of a plurality of splice variants of a transcription modulator, wherein the presence or absence of expression of each splice variant is indicative of a cancer subtype, and further comprise administering to the patient a bioactive agent capable of inhibiting the activity of one or more such splice variants determined to be expressed. In a preferred embodiment, the therapeutic agent is targeted to a basal transcription factor splice variant. In a preferred embodiment, the cancer subtype is characterized by metastatic potential. In another embodiment, the cancer subtype is characterized by its refractory behavior, particularly its non-respsonsiveness to a therapeutic agent. In another preferred embodiment, the cancer subtype is characterized by its invasive activity. In one embodiment, the methods further comprise determining the expression of other splice variants. In one embodiment, the methods further comprise determining the expression of additional markers which are useful markers of tumor cell subtypes. Examples of such markers include integrins, receptors for extracellular signals including receptor tyrosine kinases, non-receptor tyrosine kinases, matrix metalloproteinases, and other molecules known to have a role in signal transduction, cell proliferation, cell motility, cell adhesion, or cell survival.

In the treatment methods herein, the transcription modulator splice variants for which expression is determined include a plurality of basal transcription factor splice variants, which are preferably selected from those described herein. In a preferred embodiment, one or more of the basal transcription factor splice variants is derived from a gene selected from group of gene families consisting of TAF, SMARC, HDAC, MED12, NCOA, GTF, THRAP, HMG, OGDL, BRF, and BAF. In a further preferred embodiment, one or more of the basal transcription factor splice variants is derived from a gene selected from the group of gene families consisting of TAF2, TAF4, TAF6L, TAF7L, TAF8, TAF10, TAF15, SMARCA1, SMARCA2, SMARCA4, SMARCA5 SMARCB1, SMARCC2, SMARCD3, NCOA, NCOA3, NCOA4, NCOA6, NCOA7, BRF1, GTF3C, GTF2F, MED12, THRAP4, THRAP3, HMG20, OGHDL, HDAC5, AND BAF250. Especially preferred are combinations of transcription modulator splice variants described herein and splice variants of NRSF, MDM2, TSG, RREB, ZNF207, TTF-1, GTFIIIA, HES-6, HRY, Msx2, Neu, NeuroD1, Mash-1, and Irx2 which are tumor-specific/enriched, as disclosed in PCT/US03/41253.

In one aspect, the invention provides therapeutics targeted to transcription modulator splice variants associated with cancer. Preferred therapeutic targets are transcription factor splice variants, with basal transcription modulator splice variants being especially preferred. In a preferred embodiment, molecular therapeutics capable of reducing the expression of such splice variants in cancer cells are provided. Preferred molecular therapeutics include agents targeted to mRNA encoding such splice variants, such as, for example, siRNA and antisense molecules targeted to such splice variant mRNAs.

Also provided herein are novel splice variant proteins, and nucleic acids encoding the same, as well as fragments thereof, and fusion molecules comprising the novel splice variants or fragment thereof. Also provide herein are antibodies that specifically bind to the novel splice variant proteins provided herein. Also provided are peptides corresponding to novel sequences provided by the novel splice variants herein which are capable of binding to autoantibodies that specifically bind to the novel splice variant proteins provided herein.

### BRIEF DESCRIPTION OF THE DRAWINGS

FIGS. 1-11 show the sequences of splice variants of a variety of basal transcription factors.

### DETAILED DESCRIPTION OF THE PREFERRED EMBODIMENTS

The present disclosure provides methods for diagnosing cancer and cancer subtypes which generally comprise determining the expression of a plurality of tumor-specific/enriched splice variants of transcription modulators. As disclosed herein, it is the combined determination of expression of the plurality, or the overall expression pattern, that provides for the very high accuracy of the diagnostic test, and leads to the molecular identification of cancer subtypes.

“Determining the expression” of a splice variant may be done by assaying for the expression of the splice variant in some way, for example, by assaying for the presence of its encoding mRNA, or the presence of translated protein product. Alternatively, expression may be determined indirectly by assaying for indicia of the expression of a splice variant. For example, an assay for an autoantibody that specifically binds to a splice variant but not to a wildtype transcription modulator may be performed, and the results used to infer whether or not the transcription modulator splice variant is expressed.

By “wildtype transcription modulator”, and “wildtype counterpart” of a transcription modulator splice variant, is meant an isoform of a transcription modulator that is expressed in non-tumor cells, though not necessarily exclusively, and is alternatively spliced relative to a tumor-specific or tumor-enriched splice variant isoform of the transcription modulator. The wildtype isoform is often developmentally regulated. More than one isoform may satisfy these criteria for wildtype.

By “basal transcription factor”, or “general transcription factor” is meant a member of the set of transcription factors that are necessary to reconstitute accurate transcription from a minimal promoter (such as a TATA element or initiator sequence). Basal transcription factors include those transcription factors that facilitate assembly of the preinitiation complex, as well as cofactors that associate with the basal transcriptional machinery and integrate signals from regulatory transcription factors. Included among basal transcription factors are proteins that alter chromatin structure to facilitate assembly of the preinitiation complex. Though they regulate gene expression in a general sense, they are distinct from “regulatory transcription factors”, which bind to sequences farther away from the initiation site and serve to modulate levels of transcription.

By the term “substantially complementary” herein is meant a situation where a probe sequence is sufficiently complementary to the corresponding region of its target sequence and/or another probe to hybridize under the selected reaction conditions. This complementarity need not be perfect; there may be any number of base-pair mismatches that will interfere with hybridization between a probe sequence (e.g., detection region) and its corresponding target sequence or another probe. However, if the degree of non-complementarity is so great that hybridization between a probe and its target cannot occur under even the least stringent of conditions, the probe sequence is considered to be not complementary to the target sequence.

### Splice Variants

The prominent product of gene transcription is termed the primary transcript and is a precursor to mRNA. Many primary transcripts contain intervening nucleotide sequences that are not functional in the final mRNA. These intervening, non-functional sequences are called introns, while the sequences of the primary transcript that are preserved in the mature mRNA are called exons. Accordingly, introns are regions of the initial transcript that must be excised during post-transcriptional RNA processing, and exons are regions that are joined together after intron excision. This excision and joining process is called RNA splicing. The actual splicing is performed by a spliceosome, which is a large particulate complex consisting of various proteins and ribonucleoproteins such as snRNAs and snRNPs.

The spliceosome is responsible for cutting the primary transcript at the two exon-intron boundaries called the splice sites. The nucleotide bases of the splice sites on a primary transcript are always the same. The first two nucleotide bases following an exon are always GU, and the last two bases of the intron are always AG. It is important to note that the two sites have different sequences and so they define the ends of the intron directionally. They are named proceeding from left to right along the intron, that is as the 5′ (or donor) and the 3′ (or acceptor) sites.

The majority of normal genes are transcribed into a primary transcript that gives rise to a single type of spliced mRNA. In these cases, there is no variation in the splicing of the primary transcript; the same introns for each of the transcripts are spliced out. However, sometimes the primary transcripts of certain genes follow patterns of alternative splicing, where a single gene gives rise to more than one mRNA sequence.

In an embodiment of the invention, “splice variants” relate to the different mRNA sequences that are derived from the same gene as processed by a spliceosome. Accordingly, “splice variants” encompass any situation in which the single primary transcript is spliced in more than one way, and therefore includes splicing patterns where internal exons are substituted, added, or deleted. “Splice variants” also encompass situations where introns are substituted, added or deleted.

It has been discovered that mRNA splicing is changed in a tumor cell compared to a normal cell. Accordingly, the expression of splice variants in a tumor cell is in some way different from that of a normal cell. Changes in the splicing of tumor cells can be brought about by more than one way. For example, tumors can express products that are necessary for splicing (splicing factors, snRNAs and snRNPs) differently than normal cells. Changes in splicing patterns can also be related to mutations in the donor and acceptor sequences of certain genes in a tumor cell, thereby resulting in different splicing start and termination points.

The physiological activity of splice variant products (proteins) and the original product from which they are derived may differ. For example the splice variant could function in an opposite manner or not function at all. In addition, splice variations may result in changes of various properties not directly connected to biological activity of the protein. For example, a splice variant may have altered stability characteristics (half-life), clearance rate, tissue and cellular localization, temporal pattern of expression, up or down regulation mechanisms, and responses to agonists or antagonists.

### Transcription Modulators

The term “transcription modulator” or “transcriptional modulator” is to be construed broadly and in a preferred embodiment relates to factors that play a role in regulating gene expression. In some embodiments, a transcriptional modulator can aid in the structural activation of a gene locus. In other embodiments, a transcriptional modulator can assist in the initiation of transcription. In still other embodiments, a transcriptional modulator can process the transcript. The following is a non-exclusive list of possible factors that are considered to be transcriptional modulators.

Transcription modulators consist of basal transcription factors and transcription modulators that are not basal transcription factors, which are referred to herein as non-basal transcription modulators. Transcription modulators may be grouped according to their structure and/or function.

Among the basal transcription factor class of transcription modulators are factors that alter chromatin structure to permit access of the transcriptional components to the target gene of interest. One group of factors that alters chromatin in an ATP-dependent manner includes NURF, CHRAC, ACF, the SWI/SNF complex, and SWI/SNF-related (RUSH) proteins.

Another group of basal transcription factors is involved in the recruitment of TATA-binding protein (TBP)-containing and non-containing (Initiator) complexes. Examples of general initiation factors include: TFIIB, TFIID, TFIIE, TFIIF, and TFIIH. Each of these general initiation factors are thought to function in intimate association with RNA polymerase II and are required for selective binding of polymerase to its promoters. Additional factors such as TATA-binding protein (TBP), TBP-homologs (TRP, TRF2), initiators that coordinate the interaction of these proteins by recognizing the core promoter element TATA-box or initiator sequence and supplying a scaffolding upon which the rest of the transcriptional machinery can assemble are also considered basal transcription factors.

Included in another group of basal transcription factors are the TBP-associated factors (TAFs) that function as promoter-recognition factors, as coactivators capable of transducing signals from enhancer-bound activators to the basal machinery, and even as enzymatic modifiers of other proteins are also transcription modulators. Particular examples of these basal transcription factors and complexes thereof include: the TFIIA complex: (TFIIAa; TFIIAb; TFIIAg); the TFIIB complex: (TFIIB; RAP74; RAP30); the TAFIIA complex: (TAFIIAa; TAFIIAb; TAFIIAg); the TAFIIB complex: (TAFIIB; RAP74; RAP30); TAFs forming the TFIID complex (TAFI-15) (TAFII250; CIF150; TAFII130/135; TAFII100; TAFII70/80; TAFII31/32; TAFII20; TAFII15; TAFII28; TAFII68; TAFII55; TAFII30; TAFII18; TAFII105); the TAFIIE complex: (TAFIIEa; TAFIIEb); the TAFIIF complex (p62; p52; MAT1; p34; XPD/ERCC2; p44; XPB/ERCC3; Cdk7; CyclinH); the RNA polymerase II complex: (hRPB1, hRPB2, hRPB3, hRPB4, hRPB5, hRPB6, hRPB7, hRPB8, hRPB9, hRPB10, hRPB11, hRPB12); and others.

An additional group of basal transcription factors are those that act as a conserved interface between gene-specific regulatory proteins and the general transcription apparatus of eukaryotes. Typically, this type of mediator complex formed by basal transcription factors integrates and transduces positive and negative regulatory information from enhancers and operators to promoters. They typically function directly through RNA polymerase II, modulating its activity in promoter-dependent transcription. Examples of such mediators that form coactivator complexes with TRAP, DRIP, ARC, CRSP, Med, SMCC, NAT, include: TRAP240/DRIP250; TRAP230/DRIP240; DRIP205/CRSP200/TRIP2/PBP/RB18A/TRAP220; hRGR1/CRSP150/DRIP150/TRAP170, TRAP150; CRSP130/hSur-2/DRIP130; TIG-1; CRSP100/TRAP100/DRIP100; DRIP97; DRIP92/TRAP95; CRSP85; CRSP77/DRIP77/TRAP80; CRSP70/DRIP70; Ring3; hSRB10/hCDK8; DRIP36/hMEDp34; CRSP34; CRSP33/hMED7; hMED6; hSRB11/hCyclin C; hSOH1; hSRB7; and others. Additional members in this class include proteins of the androgen receptor complex, such as: ANPK; ARIP3; PIAS family (PIASa, PIASb, PIASg); ARIP4; and transcriptional co-repressors such as: the N-CoR and SMRT families (NCOR2/SMRT/TRAC1/CTG26/TNRC14/SMRTE); REA; MSin3; HDAC family (HDAC5); and other modulators such as PC4 and MBF1.

Non-basal transcription modulators may conveniently be grouped by their structure and/or biological function.

One group of such non-basal transcription modulators comprises neuronally enriched bHLHs such as: Neurogenins (Neurogenin-1/MATH4c, Neurogenin-2/MATH4a, Neurogenin-3/MATH4b); NeuroD (NeuroD-1, NeuroD-2, NeuroD-3(6)/my051/NEX1/MATH2/Dlx-3, NeuroD-4/ATH-3/NeuroM); ATHs (ATH-1/MATH1, ATH-5/MATH5); ASHs (ASH-1/MASH1, ASH-2/MASH2, ASCL-3/reserved); NSCLs (NSCL1/HENI1NSCL2/HEN2), HANDs (Hand1/eHAND/Thing-1, Hand2/dHAND/Thing-2); Mesencephalon-Olfactory Neuronal bHLHs: COE proteins (COE1; COE2/Olf-1/EBF-LIKE3, COE3/Olf-1Homol/Mmot1); and others.

Another group of such non-basal transcription modulators that are structurally related comprises the GIia enriched bHLHs, such as OLIG proteins (Olig1, Olig2/protein kinase C-binding protein RACK17, Olig3), and others; the HLH and bHLH families of negative regulators, which include Ids (Id1, Id2, Id3, Id4), DIP1, HES (HES1, HES2, HES3, HES4, HES5, HES6, HES7, SHARPs (SHARP1/DEC-2/eip1/Stra13, SHARP2/DEC-1/TR00067497_p), Hey/HRT proteins (Hey1/HRT1/HERP-2/HESR-2, Hey2/HRT2/HERP-1, HRT3), and others. There are other bHLHs that fall within this present category of transcriptional modulators, which include: Lyl family (Lyl-1, Lyl-2); RGS family (RGS1, RGSRGS2/GOS8, RGS3/RGP3); capsulin; CENP-B; Mist1; Nhlh1; MOP3; Scleraxis; TCF15; bA305P22.3; lpf-1/Pdx-1/ldx-1/Stf-1/luf-1/Gsf; and others.

Fork head/winged helix transcription factors constitute another group of structurally related non-basal transcription modulators. Examples of such proteins include BF-1; BF-2/Freac4; Fkh5/Foxb1/HFH-e5.1/Mf3; Fkh6/Freac7; and others.

HMG transcription factors constitute a further group of structurally related non-basal transcription modulators. Examples of such proteins include: Sox proteins (Sox1, Sox2, Sox3, Sox4, Sox6, Sox10, Sox11, Sox13, Sox14 Sox18, Sox21, Sox22, Sox30); HMGIX; HMGIC; HMGIY; HMG-17; and others.

Homeodomain transcription factors constitute yet another group of structurally related non-basal transcription modulators. Examples of such proteins include: Hox proteins; Evx family (Evx1, Evx2); Mox family (Mox1, Mox2); NKL family (NK1, NK3, NRx3.1, NK4); Lbx family (Lbx1, Lbx2); Tlx family (Tlx1, Tlx2, Tlx3); Emx/Ems family (Emx1, Emx2); Vax family (Vax1, Vax2); Hmx family (Hmx1, Hmx2, Hmx3); NK6 family (NRx6.1); Msx/Msh family (Msx-1, Msx-2); Cdx (Cdx1, Cdx2); Xlox family (Lox3); Gsx family (Goosecoid, GSX, GSCL); En family (En-1, En-2) HB9 family (Hb9/HLXB9); Gbx family (Gbx1, Gbx2), Dbx family (Dbx-1, Dbx-2); Dll family (Dlx-1, Dlx-2, Dlx-4, Dlx-5, Dlx-7); Iroquois family (Xiro1, Irx2, Irx3, Irx4, Irx5, Irx6); Nkx (NRx 2.1/TTF-1, NRx2.2/TTF-2, NRx2.8, NRx2.9, NRx5.1, NRx5.2); PBC family (Pbx1a, Pbx1b, Pbx2, Pbx3); Prd family (Otx-1, Otx-2, Phox2a, Phox2B); Ptx family (Pitx2, Pitx3/Ptx3), XANF family (Hesx1/XANF-1); BarH family (BarH, Brx2); Cut; Gtx; and others.

POU domain factors constitute yet another group of structurally related non-basal transcription modulators. Examples of such proteins include Brn2/XIPou2; Brn3a, Brn3b; Brn4/POU3F4; Brn5/Pou6FI; N-Oct-3; Oct-1; Oct-2, Oct2.1, Oct2B; Oct4A, Oct4B; Oct-6; Pit-1; TCFbeta1; vHNF-1A, vHNF-1B, vHNF-IC; and others.

Transcription modulators with homeodomain and LIM regions constitute yet another group of structurally related non-basal transcription modulators. Examples of such proteins include: Isl1; Lhx2; Lhx3; Lhx4; Lhx5; Lhx6; Lhx7 Lhx9; LMO family (LMO1, LMO2, LMO4); and others.

Paired box transcription factors constitute yet another-group of structurally related non-basal transcription modulators. Examples of such proteins include Pax2; Pax3; Pax5; Pax6; Pax7; Pax8; and others.

Zinc finger transcription factors constitute yet another group of structurally related non-basal transcription modulators. Examples of such proteins include: GATA family (Gata1, Gata2, Gata3, Gata4/5, Gata6); MyT family (MyT1, MyT1I, MyT2, MyT3); SAL family (HSal1, Sal2, Sall3); REST/NRSF/XBR; Snail family (Scratch/Scrt); Zf289; FLJ22251; MOZ; ZFP-38/RU49; Pzf; Mtsh1/teashirt; MTG8/CBF1A-homolog; TIS11D/BRF2/ERF2; TTF-I interacting peptide 21; Znf-HX; Zhx1; KOX1/NGO-St-66; ZFP-15/ZN-15; ZnF20; ZFP200; ZNF/282; HUB1; Finb/RREB1; Nuclear Receptors (liganded: ER family; TR family; RAR family; RXR family; PML-RAR family; PML-RXR family; orphan receptors: Not1/Nurr; ROR; COUP-TF family (COUP-TF1, COUP-TF2)) and others.

RING finger transcription factors constitute yet another group of structurally related non-basal transcription modulators. Examples of such proteins include: KIAA0708; Bfp/ZNF179; BRAP2; KIAA0675; LUN; NSPc1; Neutralized family (neu/Neur-1, Neur-2, Neur-3, Neur-4); RING1A; SSA1/RO52; ZNF173; PIAS family (PIAS-α, PIAS-β, PIAS-γ, PIAS-γ homolog); parkin family; ZNF127 family and others.

Another group of non-basal transcription modulators comprises enhancer-bound activators and sequence-specific or general repressors. Examples of these modulators include: non-tissue specific bHLHs, such as: USF; AP4; E-proteins (E2A/E12, E47; HEB/MEI; HEB2/ME2/MITF-2A,B,C/SEF-2/TFE/TF4/R8f); TFE family (TFE3, TFEB); the Myc, Max, Mad families; WBSCR14; and others.

Many non-basal transcription modulators have been described in the context of developmentally important signal transduction pathways.

For example, non-basal transcription modulators belonging to Wnt pathway have been described. Examples of such proteins include: β-catenin; GSK3; Groucho proteins (Groucho-1, Groucho-2, Groucho-3, Groucho-4); TCF family (TCF1A, B, C, D, E, F, G/LEF-1; TCF3; TCF4) and others.

Additionally, non-basal transcription modulators have been described in the TGFβ/BMP pathway. Examples of such proteins include: Chordin; Noggin; Follistatin; SMAD proteins (SMAD1, SMAD2, SMAD3, SMAD4, SAMD5, SMAD6, SMAD7, SMAD8, SMAD9, SMAD10); and others.

Additionally, non-basal transcription modulators have been described in the Notch pathway. Examples of such proteins include: Delta, Serrate, and Jagged families (Dll1, Dll3, Dll4, Jagged1, Jagged2, Serrate2); Notch family (Notch1, Notch2, Notch3, Notch4, TAN-1); Bearded family (E(spl)ma, E(spl)m2, E(spl)m4, E(spl)m6); Fringe family (Mfng, Rfng, Lfng); Deltex/dx-1; MAML1; RBP-Jk/CBF1/Su(H)/KBF2; RUNX; and others.

Additionally, non-basal transcription modulators have been described in the Sonic hedgehog pathway. Examples of such proteins include: SHH; IHH; Su(fu); GLI family (GLI/GLI1, Gli2, Gli3); Zic family (Zic/Zic1, Zic2, Zic3); and others.

Another group of non-basal transcription modulators includes proteins that are involved in recombination and recombinational repair of damaged DNA and in meiotic recombination. Examples of such proteins include: PCNA; RPA (RPA 14 kD, RPA binding co-activator); RFC(RFC 140 kD, RFC 40 kD, RFC 38 kD, RFC 37 kD, RFC 36 kD, RFC/activator homologue RAD17); RAD 50 (RAD 50, RAD 50 truncated, RAD 50-2); RAD 51 (RAD 51, RAD 51 B, RAD 51 C, RAD 51 C truncated, RAD 51 D, RAD 51 H2, RAD 51 H3, RAD 51 interacting/PIR 51, XRCC2, XRCC3); RAD 52 (RAD 52, RAD 52 beta, RAD 52 gamma, RAD 52 delta); RAD 54 (RAD 54, RAD 54 B, RAD 54, ATRX); Ku (Ku p70/p80); NBS1 (nibrin); MRE11 (MRE11, MRE11A, MRE11B); XRCC4; and others.

Another group of non-basal transcription modulators includes proteins relating to cell-cycle progression-dedicated components that are part of the RNA polymerase II transcription complex. Examples of these proteins include: E2F family (E2F-1, E2F-3, E2F-4, E2F-5); DP family (DP-1, DP-2); p53 family (p53, p63; p73); mdm2; ATM; RB family (RB, p107, p130).

Still another group of non-basal transcription modulators includes proteins relating to capping, splicing, and polyadenylation factors that are also a part of the RNA polymerase II modulating activity. Factors involved in splicing include: Hu family (HuA, HuB, HuC, HuD); Musashi1; Nova family (Nova1, Nova2); SR proteins (B1C8, B4A11, ASF SRp20, SRp30, SRp40, SRp55, SRp75, SRm160, SRm300); CC1.3/CC1.4; Def-3/RBM6; SIAHBP/PUF60; Sip1; C1QBP/GC1Q-R/HABP1/P32; Staufen; TRIP; Zfr; and others. Polyadenylation factors include: CPSF; Inducible poly(A)-Binding Protein (U33818), and others.

Another group of non-basal transcription modulators includes protein kinases. Examples of these proteins include: AGC Group: AGC Group I (cyclic nucleotide regulated protein kinase (PKA & PKg) family); AGC Group II (diacylglycerol-activated/phospholipid-dependent protein kinase C (PKC) family); AGC Group III (related to PKA and PKC (RAC/Akt) protein kinase family); AGC Group IV (kinases that phosphorylate ribosomal protein S6 family); AGC Group V (budding yeast AGC-related protein kinase family); AGC Group VI (kinases that phosphorylate ribosomal protein S6 family); AGC Group VII (budding yeast DB 2/20 family); AGC Group VIII (flowering plant PVPk1 protein kinase homologue family); AGC Group Other (other AGC related kinase families); CaMK Group: CaMK Group I (kinases regulated by Ca2+/CaM and close relatives family); CaMK Group II (KIN1/SNF1/Nim1 family); CaMK Other (other CaMK related kinase families); CMGC Group: CMGC Group I (cyclin-dependent kinases (CDKs) and close relatives family); CMGC Group II (ERK (MAP) kinase family); CMGC Group III (glycogen synthase kinase 3 (GSK3) family); CMGC Group IV (casein kinase II family); CMGC Group V (Clk family); CMGC Group Other; Protein-tyrosine kinases (PTK): A. non-membrane spanning: PTK group I (Src family); PTK group 11 (Tec/Akt family); PTK group III (Csk family); PTK group IV Fes (Fps) family; PTK group V (AbI family); PTK group VI (Syk/ZAP70 family); PTK group VIII (Ack family); PTK group IX (focal adhesion kinase (Fak) family); B. membrane spanning: PTK group X (epidermal growth factor receptor family); PTK group XI (Eph/Elk/Eck receptor family); PTK group XII (Axl family); PTK group XIII (Tie/Tek family); PTK group XIV (platelet-derived growth factor receptor family); PTK group XV (fibroblast growth factor receptor family); PTK group XVI (insulin receptor family); PTK group XVII (LTK/ALK family); PTK group XVIII (Ros/Sevenless family); PTK group XIX (Trk/Ror family); PTK group XX (DDR/TKT family); PTK group XXI (hepatocyte growth factor receptor family); PTK group XXII (nematode Kin15/16 family); PTK other membrane spanning kinases (other PTK kinase families); OPK Group: OPK Group I (Polo family); OPK Group II (MEK/STE7 family); OPK Group III (PAK/STE20 family); OPK Group IV (MEKK/STE11 family); OPK Group V (NimA family); OPK Group VI (wee1/mik1 family); OPK Group VII (kinases involved in transcriptional control family); OPK Group VIII (Raf family); OPK Group IX (Activin/TGFb receptor family); OPK Group X (flowering plant putative receptor kinases and close relatives family); OPK Group XI (PSK/PTK “mixed lineage” leucine zipper domain family); OPK Group XII (casein kinase I family); OPK Group XIII (PKN prokaryotic protein kinase family); OPK Other (other protein kinase families).

Another group of non-basal transcription modulators includes cytokines and growth factors. Examples of these proteins include: Bone morphogenetic proteins: Decapentaplegic protein (Dpp), BMP2, BMP4; 60A, BMP5, BMP6, BMP7/OP1, BMP8a/OP2 BMP8b/OP3; BMP3 (Osteogenin), GDF10; BMP9, BMP10, Dorsalin-1; BMP12/GDF7 BMP13/GDF6; GDF5; GDF3Ngr2; Vg1, Univin; BMP14, BMP15, GDF1, Screw, Nodal, XNrl-3, Radar, Admp; Cytokines: Ciliary neurotrophic factor (CNTF) family; Leukemia inhibitory factor; Cardiotrophin-1; Oncostatin-M; Interleukin-1 family; Interleukin-2 family; Interleukin-3 (IL-3); Interleukin-4 (IL-4); Interleukin-5 (IL-5) family; Interleukin-6 (IL-6) family; Interleukin-7 (IL-7); Interleukin-9 (IL-9); Interleukin-10 (IL-10); Interleukin-11 (IL-11); Interleukin-12 (IL-12); Interleukin-13 (IL-13); Interleukin-15 (IL-15) family; GM-CSF; G-CSF; Leptin; Epidermal growth factors: Amphiregulin; Acetylcholine receptor-inducing activity (ARIA); Heregulin (Neuregulin) (NEU differentiation factor); Transforming growth factor α (TGF-α) family; Neuregulin 2; Neuregulin 3; Netrin 1 and 2; Fibroblast growth factors (FGF): FGF-1 (acidic); FGF 2 (basic); FGF3/int-2 (murine mammary tumor virus integration site (v-int-2) oncogene homolog); FGF4/transforming gene from human stomach-1/hst/hst-1/heparin-binding secretary transforming factor-1 (HSTF1)/Kaposi's sarcome FGF (ksFGF)/K-FGF/KS3; FGF5/oncogene encoding fibroblast growth factor-related protein; FGF6/fibroblast growth factor-related gene/hst-2; FGF7, keratinocyte growth factor (KGF); FGF8/androgen-induced growth factor (AIGF); FGF9/glia-activating factor (GAF); FGF10/keratinocyte growth factor 2, KGF-2; FGF11/fibroblast growth factor homologous factor 3 (FHF-3); FGF12/fibroblast growth factor homologous factor 1 (FHF-1); FGF13/fibroblast growth factor homologous factor 2 (FHF-2); FGF14/fibroblast growth factor homologous factor 4 (FHF-4); FGF15; FGF16; FGF17/FGF13; FGF18; FGF19; FGF20/XFGF-20; FGF21; FGF22; FGF23; FGFH/fibroblast growth factor homologous; C05D11.4/hypothetical 48.1 KD protein COD11.4; GDNF: Artemin; Glial-derived neurotrophic factor (GDN F); Neurturin; Persephin; Heparin-binding growth factors: Pleiotrophin (NEGF1); Midkine (NEGF2), Insulin-like growth factors (IGF): Insulin-like IGF1 and IGF2; Neurotrophins: Nerve growth factor (NGF); Brain-derived neurotrophic factor (BDNF); Neurotrophin-3 (NT-3); Neurotrophin-4/5 (NT-4/5); Neurotrophin-6 (NT-6) family; Tyrosine kinase receptor ligands: Stem cell factor; Agrin; FLT3L; Macrophage colony stimulating factor-1 (CSF-1); Platelet derived growth factor (PDGF) family; Other: Hedgehog family (Indian hedgehog (Ihh), Desert Hedgehog (Dhh), Sonic Hedgehog (Shh)); Wnt Group: WNT1/INT; WNT2/IRP, WNT2B/13; WNT3; WNT3A; WNT4; WNT5A, WNT5B; WNT6; WNT7A, WNT7B; WNT8A/WNT8d, WNT8B; WNT10A, WNT10B; WNT11; WNT14; WNT15; WNT16 isoforms; negative regulators of Wnt signaling: Dickkopf (Dkk) family (Dkk1, Dkk2, Dkk3, Dkk4); Frisbee; Cerberus; Wnt binding factors: WIFs.

Non-basal transcription modulators may be further subdivided into groups of non-basal transcription factors, and transcription modulators that are non-transcription factors. An exemplary group of transcription factors is the group of bHLH factors (e.g., NeuroD) involved in neuronal development. An exemplary group of transcription modulators that are non-transcription factors is the kinase group of factors, discussed above. Transcription factors, in general, access the nucleus and are capable of impacting transcription and gene expression through DNA interactions. These DNA interactions may be direct or indirect. Disease-associated splice variants of transcription factors, and especially of basal transcription factors, are the preferred targets for therapeutics disclosed herein.

### Methods and Compositions for Cancer Diagnosis

Disclosed herein are methods and compositions for diagnosing cancer. The methods generally comprise determining the expression of a plurality of tumor-specific/enriched splice variants, particularly a plurality of basal transcription modulators. In a preferred embodiment, the methods comprise determining the expression of at least one splice variant of a plurality of transcription modulators, wherein the expression of each splice variant is indicative of cancer. In another preferred embodiment, the methods comprise determining the expression of a plurality of splice variants of at least one transcription modulator.

While the expression of each of the splice variants is indicative of cancer, each is not necessarily expressed in every occurrence of a particular cancer or in every cancer type. Moreover, all splice variants for which expression is determined in a diagnostic assay that gives a result indicative of cancer are not necessarily expressed. Rather, it is the determination of the overall expression pattern of a plurality of tumor-specific/enriched splice variants that provides for the very high accuracy of the subject diagnostic methods. Further, as also exemplified herein, the determination of negative expression results for transcription modulator splice variants in some samples in a cancer group yields the molecular identification of cancer subtypes.

Disclosed herein are sets of transcription modulator splice variants that are tumor-enriched or tumor-specific, the expression of which can be determined, and such a determination used as a highly accurate indicator of cancer. While these particular splice variants are of tremendous utility, other tumor-specific/enriched splice variants are contemplated for use in the subject methods. It will be appreciated by the artisan that by increasing the number of tumor-specific/enriched splice variants for which expression is determined, the accuracy of the subject methods is increased, and, importantly, cancer subtypes are more clearly defined, and new subtypes are revealed. All of these factors are beneficial to the effective treatment of cancer.

In addition, it will be appreciated by the artisan that the number of tumor-specific/enriched splice variants for which expression is determined can easily be increased to the point where a single, simultaneous expression determination, or a series of expression determinations, is sufficient to diagnose any of a large number of cancer types and subtypes.

Accordingly, the disclosed methods are useful for diagnosing the existence of a neoplasm or tumor of any origin. For example, the tumor may be associated with lung cancer (e.g., small cell lung cancer, non-small cell lung cancer), gastrointestinal cancer (e.g., colorectal cancer, stomach cancer, liver cancer, pancreatic cancer, and cancers of other regions of gastrointestinal tract), breast cancer, prostate cancer, skin cancer (e.g., basal cell carcinoma, melanoma), sarcoma, endocrine cancer (e.g., carcinoids, insulinoma, cancer of thyroid gland), neural cancers (e.g., neuroblastoma, glioblastoma, medulloblastoma, retinoblastoma), bladder cancer, cervical cancer, renal cancer, hematopoietic cancers (e.g., lymphoma, leukemia). In addition to diagnosing general types of tumors, it is a preferred embodiment of the current invention to diagnose molecular subtypes of the above-listed neoplasia and tumors.

In a preferred embodiment of diagnosing a tumor a practitioner could use primers provided herein to detect the expression of tumor-specific/enriched transcriptional modulator splice variants. In another preferred embodiment, a practitioner could diagnose cancer from neoplastic cells from one of the following sources: blood, tears, semen, saliva, urine, tissue, serum, stool, sputum, cerebrospinal fluid and supernatant from cell lysate. However, diagnosis of a tumor can be performed with as few as one tumor cell from any sample source.

The determination of splice variant isoform expression and its distinction from wildtype expression may be accomplished in a number of ways. With respect to autoantibody detection, when alternative splicing produces a splice variant with a coding sequence that differs from the wildtype isoform, peptides unique to the splice variant isoform (i.e., not present in wildtype isoform) may be used to probe patient sera for the presence of autoantibodies that specifically recognize the peptide, where the presence of such antibodies is indicative of the presence of the splice variant irrespective of the presence of the wildtype isoform of the transcription modulator.

With respect to mRNA detection, RT-PCR reactions may be designed to distinguish the presence of splice variant mRNA from wildtype mRNA. In one embodiment, where alternative splicing removes nucleotide sequence present in the wildtype transcript, primers complementary to mRNA sequence adjacent to the splice junction site in the splice variant may be used to generate a PCR product that traverses the junction site to produce a first product, where the same primers would produce a second product of a different size when reacted with a wildtype transcript. PCR products may be distinguished, for example, by size, and the expression of splice variant mRNA may be discerned from the presence of the splice variant-derived PCR product. In another embodiment, where alternative splicing adds sequence not present in the wildtype construct, primers complementary to mRNA sequence adjacent to each of two splice junctions in a splice variant (between which non-wildtype sequence resides) may be used to generate a PCR product that traverses the junction sites of the splice variant to produce a first product, where the same primers would produce a second product of a different size when reacted with a wildtype transcript. Again, PCR products may be distinguished and the expression of splice variant mRNA determined. Alternatively, a first primer complementary to mRNA sequence adjacent to one of the splice junctions may be used with a second primer complementary to a segment of the non-wildtype sequence present in the splice variant. In this case, the second primer would not hybridize to the wildtype construct, and the PCR reaction would only produce a product in the presence of the splice variant. In preferred embodiments, the mRNA sequence adjacent to the splice junction(s) of interest may optimally be within about 50 to about 100 nucleotides of the splice junction(s), though it will be appreciated by the skilled artisan that greater and shorter distances from the splice junction(s) may be used, and such distances are embraced by other embodiments.

PCR methods are well known in the art. For example, see Current Protocols in Molecular Biology, Greene Pub. Associates and Wiley-Interscience; New York; Eds. Ausubel et al., 1988/April 2003, Chapter 15, The Polymerase Chain Reaction.

Preferred transcription modulator splice variants for which expression is determined include those set forth below. In some cases, primer sequences useful for amplifying and obtaining the varied sequences are presented. It will be appreciated that primer design is routine in the art, and that by disclosing the variation of a splice variant, one of skill in the art would be capable of designing appropriate amplification primers without undue experimentation.

| | | | | |
|---|---|---|---|---|
| gene/ASV | cDNA | protein aa | forward primer | reverse primer |

| | | | | |
|---|---|---|---|---|
| TAF2 | NM_003184 | 1199 | | |
| TAF2 ASV1 | insert 165 nt after ex. | 432 | 5′-TTGGTTCCCTTGTGTTGATTC | 5′ TGGAAACCAACATCTGACTCC |
| (S2/AS2) | 9 | | | |
| TAF2 ASV2 | insert 152 nt after ex. | 409 | 5′-TTGGTTCCCTTGTGTTGATTC | 5′ TGGAAACCAACATCTGACTCC |
| | 9 | | | |
| TAF4 | NM_003185 | 1083 | | |
| (S2/AS3) | | | | |
| TAF4 ASV1 | exons 6-9 spliced out | 628 | 5′-GACCAACATCCAGAACTTCCA | 5′-TGCTTTG AGAGCAGCAGTGA |
| TAF4 ASV2 | exon 7 spliced out | 1000 | 5′-GACCAACATCCAGAACTTCCA | 5′-TGCTTTG AGAGCAGCAGTGA |
| (S2/AS2) | | | | |
| TAF4 ASV3 | exons 6, 7 spliced out | ORF continues, | | |
| | | 970 aa | | |
| TAF4 ASV4 | part of exon 7, 8 | ORF continues, | | |
| | spliced out | 1015 aa | | |
| TAF4 ASV5 | deletion in exon 1 | 452 aa missing | | |
| | (65-1355) | in NH terminus, | | |
| | | 630 aa | | |
| TAF4 ASV6 | combination of ASV2 and | 547 aa | | |
| | ASV5 | | | |
| TAF6L | NM_006473 | | | |
| TAF6L ASV1 | unspliced intron between | truncated protein | | |
| | exons 5 and 6 | 161 aa | | |
| TAF6L ASV2 | unspliced intron between | truncated protein | | |
| | exons 5 and 6 | 157 aa | | |
| TAF7L | NM_024885 | 303 | | |
| TAF7L ASV1 | new exon between ex. 8 | 375 | 5′- | 5′-CATAAGGCAACTGAAGGGACA |
| | and 9 | | AGACATGAGTGAAAGCCAGGA | |
| TAF8 | NM_138572 | 259 aa | | |
| TAF8 ASV1 | exons 6-8 spliced out | truncated protein | | |
| | | 214 aa | | |
| TAF8 ASV2 | different exons after 7, | different COOH | | |
| | 9 is similar | terminus | | |
| | | 310 aa | | |
| TAF8 ASV3 | exons 5 and 6 spliced | truncated protein | | |
| | out | 168 aa | | |
| TAF10 | NM_024885 | 218 | | |
| TAF10 ASV1 | intron seq. after exon 2 | 138 | 5′-GGCCATATCTAACGGGGTTTA | 5′-GGGACATGGGGACAGATAAGT |
| TAF10 ASV2 | intron seq after exon 4 | 198 | 5′-GGCCATATCTAACGGGGTTTA | 5′-GGGACATGGGGACAGATAAGT |
| TAF10 ASV3 | intron after exon 2 and | 138 | 5′-GGCCATATCTAACGGGGTTTA | 5′-GGGACATGGGGACAGATAAGT |
| | exon 4 | | | |
| TAF10 ASV4 | intron after exon 2 | truncated protein | | |
| | | 138 aa | | |
| TAF15 | NM_139215 | 592 | | |
| (S2/AS2) | | | | |
| TAF15 ASV1 | exon 15 spliced out | 485 | 5′-TTGATGACCCTCCTTCAGCTA | 5′-GCAAAACTCTGGCAATTTCAC |
| SMARCA1 | NM_003069 | 1054 | | |
| (S3/AS2) | | | | |
| SMARCA1 | exon 13 is spliced out | 1043 | 5′- | 5′-AGGTTAATTCCGAGACCTCCA |
| ASV1 | | | AGATGACTCGCTTGCTGGATA | |
| SMARCA2 | NM_003070 | 1586 | | |
| (S6/AS6) | | | | |
| SMARCA2 | deletion in ex 29 | 15668 | 5′- | 5′-TGAAATCCACTGGCTTCCTAA |
| ASV1 | | | CTGAGGCTCTGTACCGTGAAC | |
| SMARCA4 | NM_003072 | 1647 | | |
| (S6/AS6) | | | | |
| SMARCA4 | exon 27 is out (fragment | 1614 | 5′-ACCACAAAGTGCTGCTGTTCT | 5′-TTCTTCTGCTTCTTGCTCTCG |
| ASV1 | 950) | | | |
| SMARCA5 | NM_003601 | 1052 aa | | |
| SMARCA5 | exons 1-3 partially | 933 aa, first | | |
| ASV1 | spliced out (222-794) | 119 aa missing | | |
| SMARCA5 | deletion in exon1 (nt | 969 aa protein, | | |
| ASV2 | 235-640) | first | | |
| | | 83 aa missing | | |
| SMARCB1 | NM_003073 | 385 | | |
| SMARCB1 | Deletion in exon 2 (nt | 376 | 5′-ATTTCGCCTTCCGGCTTC | 5′-TACTTCTCATCGTTGCCATCC |
| ASV1 | 355-378) | | | |
| SMARCC2 | NM_003075 | 1214 | | |
| (S5/AS5) | | | | |
| SMARCC2 | nt 3255-3600 spliced in | 1099 | 5′- | 5′-AGATGTCTGGCTGGCTCCT |
| ASV1 | exon 27 | | CTGCTGTTGAGGAAAGGAAGA | |
| SMARCC2 | nt 3255-3531 spliced in | 1121 | 5′- | 5′-AGATGTCTGGCTGGCTCCT |
| ASV2 | exon 27 | | CTGCTGTTGAGGAAAGGAAGA | |
| SMARCC2 | extra ex. between 17 and | 1245 | 5′- | 5′-CGGACACTTTGTTCCAGTCAT |
| ASV3 | 18 | | AACCCCAGAAGCAAAGAAGAA | |
| SMARCC2 | extra exon after 17 and | 1131 aa | | |
| ASV4 | deletion exon 27 | | | |
| SMARCD3 | NM_003078 | 470 | | |
| SMARCD3 | New ORF or short trunc | 382 | 5′- | 5′-ACTTTTAATCCAGCCCCACAC |
| ASV1 | | | ATGACTCTCCAGGTGCAGGAC | |
| SMARCD3 | ex.s 3, 4, 5 out | 344 | 5′- | 5′-ACTTTTAATCCAGCCCCACAC |
| ASV2 | | | ATGACTCTCCAGGTGCAGGAC | |
| NCOA2 | NM_006540 | 1465 | | |
| (S2/AS2) | | | | |
| NCOA2 ASV1 | ex 13 spliced out | 1385 | 5′-TAGCCAGCTCTTTGTCGGATA | 5′-AGGAGAGCTCCCTCATCACTC |
| NCOA3 | NM_181659 | 1425 aa | | |
| NCOA3 ASV1 | 3145-(3950-3980) out in | 1052 aa, poly Q | | |
| | strech of CAG | at the COOH | | |
| | | terminus | | |
| NCOA4 | NM_005437 | 615 | | |
| (S1/AS2) | | | | |
| NCOA4 ASV1 | exon 8 out | 286 | 5′- | 5′-GGTCAGACCCAGAAACACAA |
| | | | GAGGGACTTGGAGCTTGCTAT | |
| NCOA6 | NM_014071 | 2064 | | |
| (S2/AS2) | | | | |
| NCOA6 ASV1 | deletion beginning of ex | 568 | 5′-GCCACCTCAAAATAACCCACT | 5′-GGTTCTGAGGGTTCAAGGTTC |
| | 8 | | | |
| NCOA7 | NM_181782 | 943 | | |
| (S1/AS1) | | | | |
| NCOA7 ASV1 | exon 3 out | 877 | 5′- | 5′-CAATGGAAACAACCTCTTCCA |
| | | | GAGAAGAAGGAACGGAAACAAA | |
| GTF3C5 ASV1 | Exon skipping + alterna- | | AGTGGTGCGTGATGTGGCTAAG | GCTTGAAGTCCTCCTCCTCCTCT |
| | tive exon, deleted (exon | | A | |
| | IV partly + exonV en- | | | |
| | tirely) + additional | | | |
| | exon VIII | | | |
| BRF1 | Exon skipping, exons 5- | | GGTCATCAGTGTGGTCAAAGTG | GCTGAGACCTCCTACGAGTGGTAC |
| | 11 deleted, deletion in | | | |
| | exon 12 | | | |
| GTF2F1 asv1 | Exon skipping + cryptic | | CGTCCTACTACATCTTCACCC | CTCTTGGGTGGCGTCTTCTTC |
| | splicing, deletion in | | | |
| | exon 5, cryptic splic- | | | |
| | ings in exons 4 and 6, | | | |
| | deletion 396 nt | | | |
| GTF2F1 asv2 | intron retained between | | | |
| | exons 10 and 11, | | | |
| | insertion 79 nt | | | |
| MED12 | Gene ID 9968 | | | |
| MED12 ASV1 | introns 8, 11 unspliced | | | |
| MED12 ASV2 | intron 18 unspliced | | | |
| MED12 ASV3 | Deletion from mid-exon | | | |
| | 11 through mid-exon 19 | | | |
| MED12 ASV4 | Intron 21 unspliced AND | | | |
| | exon 22 truncated on | | | |
| | 3′end by 31 nt (net | | | |
| | increase of 394 nt) | | | |
| MED12 ASV5 | Intron 21 unspliced re- | | | |
| | sulting in 425 nt | | | |
| | increase | | | |
| MED12 ASV6 | Large deletion from mid- | | | |
| | exon 11 through exon 21, | | | |
| | with exon 19 redefined. | | | |
| | Also, exon 21 through | | | |
| | exon 24 (end of clone) | | | |
| | is intact, with no in- | | | |
| | trons spliced out | | | |
| MED12 ASV7 | Intron 24 unspliced re- | | | |
| | sulting in 395 nt | | | |
| | increase | | | |
| MED12 ASV8 | Intron 39 unspliced re- | | | |
| | sulting in 174 nt | | | |
| | increase | | | |
| MED12 ASV9 | First: Intron 39 un- | | | |
| | spliced resulting in 174 | | | |
| | nt increase; Second: | | | |
| | exon 41 has internal | | | |
| | intron splice out | | | |
| | (known ASV) which de- | | | |
| | letes 75 nts | | | |
| MED12 ASV10 | Exon 20 extended 3′, | | | |
| | resulting in a 109 nt | | | |
| | increase | | | |
| THRAP4 | gene id 9862 | | | |
| THRAP4 ASV1 | Extra 57 nt exon between | | | |
| | exons 6 and 7 | | | |
| THRAP4 ASV2 | First: extra exon be- | | | |
| | tween exons 6 and 7, (57 | | | |
| | nt); exon 7 is extended | | | |
| | on the 5′ end by 315 nts | | | |
| THRAP3 | gene id 9967 | | | |
| THRAP3 ASV1 | Extra exon (192 nt), lo- | | | |
| | cated 114 nt after exon | | | |
| | 8 | | | |
| HMG20B | gene id 10362 | | | |
| HMG20B ASV1 | Exon 5 spliced out, loss | | | |
| | of 216 nt | | | |
| OGHDL | gene id 55753 | | | |
| OGHDL ASV1 | exon 10 extended 5′ | | | |
| HDAC5 ASV1 | Alternative exon, exons | | GAGGAGGATTGCATCCAGGT | TCCTCCACCAACCTCTTCAG |
| | 14 and 15 in; insertion | | | |
| | 255 nt | | | |
| BAF250 ASV1 | Exon skipping, exon 16 | | CCCAGCCAGCAGACTACAATG | CTAATGCCCATGTGCTCTCTG |
| | deleted, deletion 892 nt | | | |
| BAF250 ASV2 | deletion in exon 16, | | | |
| | deletion of 651 nt | | | |

TABLE 2

Non-basal Transcription Modulator Splice Variants - Transcription Factors

| | | | | |
|---|---|---|---|---|
| GROUP | Symbol | Splicing Type | Sense | Asense |

| | | | | |
|---|---|---|---|---|
| TF | AKNAh | Alternative exon, additional exon | ATGGCTGGCTACGAATACG; | as1GCTACGAAGTTGAGGATGCC; |
| | | after 1 exon | | as2GCACCTCCCTTTCATCTGGT |
| TF | Alx4 | Cryptic splicing, deletion in 3′UTR | CCCACTCGACTTTCCTCTTAG | ACTAGGCAGAGCAGAGGAGTGG |
| TF | ANAC | Alternative exon, 3 additional al- | CTACAGAGCAGGAGTTGCCGC | GCTGCAGTTACTCCTTTGAGACACCA |
| | | ternative exons after exon 1 | AG | |
| TF | AP-4 | Cryptic splicing, deletion in exon | CCACCACTTGTATCCAGCACCC | CGCTGGTGTGTGATGGGTAC |
| | | 14 | | |
| TF | ARNT | Exon skipping, exons 12-20 deleted. | GATGGGGAACCTCACTTCGTGG | CTCCCAGCATGGACAGCATCTC |
| TF | ATF3 | Alternative exon, additional exon | GGGGTGTCCATCACAAAAGCC | ATGGGAAGGGCCTGCTGAATC |
| | | before exon 4 | GAG | |
| TF | BIN1 | Exon skipping, exons 12 and 13 | CTGCAAAAGGGAACAAGAGCC | AGGGTTCTGGAAGGGGATCAC |
| | | deleted. | | |
| TF | CTDP1 | Alternative exon | TGCCAAGTATGACCGCTACCTC | AGAAAGCAGCGTGGACCGAGACTG |
| | | | AACA | |
| TF | CUX | Alternative exon, alternative | GCTATTTTCAGGCACGGTTTCT | TCCACATTGTTGGGGTCGTTC |
| | | transcription initiation between | C | |
| | | exons 20 and 21. | | |
| TF | TELF1 | Intron retention. | GACTAGAATATCAATGAACCAG | GCAGTGCCAGTAAAAACTCCC |
| | | | G | |
| TF | ELF3 | Alternative exon, different 5′ UTR | s1CCTGGCGGAACTGGATTTCT | as1CTGTACCCTCCAATGACATCG, |
| | | | CTC, | as2GGAAGAGCTTGCCATCAGTG |
| | | | s2GTTGGATCATTGAGCTGCTG | |
| | | | G | |
| TF | ER1 | Alternative exon, exon 2 inserted. | TGCCCTACTACCTGGAGAAC | as1CTGATGTGGGAGAGGATGAGGA, |
| | | | | as2GCTCTGTTCTGTTCCATTGGTC |
| TF | FXR1 | Exon skipping, exon 15 spliced out | GAAGAGGCAGAAGTGTTTCAGG | TGGAGGAACTGAAAGTGCGATG |
| | | | G | |
| TF | GATA1 | Cryptic splicing, deletion in exon 6 | TGTCAGTAAACGGGCAGGTAC | CTGGCTACAAGAGGAGAAGGAC |
| TF | Gli2 | Cryptic splicing, deletion in exon 5 | AACAAGCAGAGCAGTGAGTCG | GGCACACAAACTCCTTCTTCTCCC |
| | | | G | |
| TF | Hes6 | Cryptic splicing, deletion in exon 3 | s1TGCTGGCGGGCGCCGAGGT | GCATGGACTCGAGCAGATGGTTC |
| | | | GCA, | |
| | | | s2TGCTGCTGGCGGGCGCCGA | |
| | | | GGCC | |
| TF | HesR1 | Cryptic splicing, exon 3 longer, | s1TTCTTTTGGGGGGAGGGGAA | as1GCTCAGATAACGCGCAACTTC, |
| | | deletion in 3′UTR | C, | as2CTCAATTGACCACTCGCACACC |
| | | | s2GCTTTTGAGAAGCAGGGATC, | |
| | | | s3TGAGAAGCAGGTAATGGAGC | |
| TF | HOXA1 | Cryptic splicing, two deletions in | GTCCTACTCCCACTCAAGTTG | CTCCTTCTCCAGTTCCGTGAGC |
| | | exon 1 | | |
| TF | HRY | Cryptic splicing, deletion in exon 1 | s1AAATTCCTCGTCCCCCGGTC | CGGAGGTGCTTCACTGTCATTTCC |
| | | | AGC; | |
| | | | s2AAATTCCTCGTCCCCGGTCA | |
| | | | GC | |
| TF | HSSB | Cryptic splicing, alternative splice | TGGCTGGGCTGCTCGGGTTAG | CTCCTTCTCTTTCGTCTGGTCACTC |
| | | donor in exon 1. Probably leads to | A | |
| | | an mRNA that is not translated. | | |
| TF | Mdm-2 | Exon skipping, exons 4-11 spliced | TGCTGTAACCACCTCACAG | CACACTCTCTTCTTTGTCTTGGG |
| | | out | | |
| TF | MITF | Alternative exon, different 5′ re- | GTGCAGACCCACCTCGAAAACC | as1CCAGACATTCACAACAAGCGGAA |
| | | gion, additional exon between exons | | C, |
| | | 3 and 4. | | as2GGACGCTCGTGAATGTGTGTTC |
| TF | MOX1 | Cryptic splicing, exon 2 deleted | AGGGGGTTCCAAGGAAATGGG | TGACCTCCCTTCACACGCTTCC |
| TF | nfkb2 | Alternative exon + exon skipping, | GCCTGACTTTGAGGGACTGTAT | CCTCCCCTTCCCATGAGAATCC |
| | | alternative exons 18, 19 and exons | CC | |
| | | 18-22 spliced out. | | |
| TF | Oct1 | Exon skipping, exon 2 in, exon 3 | GGAGGAGCAGCGAGTCAAGAT | GCCTGGGCTGTTGAGATTGC |
| | | deleted, exon 5 in. | G | |
| TF | Oct2 | Cryptic splicing, deletion in exon | CCAGCTACAGCCCCCATATG | GATTCCCGCTGCCATCAAGG |
| | | 13 | | |
| TF | OIP2 | Cryptic splicing, alternative splice | AGATGGTTCTGCTTTAGTGAAG | GTCATCAAACACAGCAAAGGAAG |
| | | acceptor in exon 6 | TTGG | |
| TF | PAX2 | Exon skipping + Alternative exon, | TTTCCAGCGCCTCCAATGACCC | GTCGGCCTGAAGCTTGATGTGG |
| | | alternative exon 6, exon 10 deleted. | | |
| TF | PCNP | Exon skipping, exons 2 and 3 spliced | AAATGGCGGACGGGAAGGC | AAAGCGGCTCCAAAGATAGTC |
| | | out. | | |
| TF | PGR | Exon skipping, exon 4 deleted. | ATGGTGTCCTTACCTGTGGGAG | TACAGCATCTGCCCACTGAC |
| TF | SCRAP | Exon skipping, exon 23 deleted. | GCAAACCTCTCACCTTCCAAAT | TGGAAGCCCAGAGCTCGGA |
| | | | C | |
| TF | TCF3 | Exon skipping, exons III & IV | s1CAGGAGAATGAACCAGCCGC | as1CCTCGTCCAGGTGGTCTTCTATC; |
| | | deleted | AGA, | as2GCTGCTTTGGGATTCAGGTTCC |
| | | | s2GCAATAACTTCTCGTCCAGCC | |
| | | | CTT | |
| TF | Trim19 | Exon skipping + cryptic splicing, | CAACAACATCTTCTGCTCCAAC | TCACTGGACTCACTGCTGCTGTCAT |
| | lambda | exon IV deleted, exon V partly | CC | |
| | | deleted | | |
| TF | WT1 | Cryptic splicing, deletion in exon 9 | CCCAGCTTGAATGCATGACCTG | TTGGCCACCGACAGCTGAAG |
| | | | G | |
| TF | ZNF147 | Exon skipping, exon 6 deleted. | CTGCGAGGAATCTCAACAAAGC | AGGAAGGTCTCCAGCACCTTGG |
| | | | C | |
| TF | ZNF398 | Exon skipping + Alternative exon, | s1ATCTTGGCTCACTGCAACCTC | GTGTGCCTCATTTGCTGCTGGG |
| | | different 5′ exon, exon 3 in. | CG; | |
| | | | s2TAGACAGCGCAGGGCCATGG | |
| TF | SMARCD1 | Alternative exon + exon skipping, | s1GGCGGGTTTCCAGTCTGTGG | CTGTAATCCAGCATCAGTAGGACA |
| | | exon 1 different + Exon 5 deleted | CTC, | |
| | | | s2CTATCCGAGACCAGGTATGTT | |
| | | | GC | |
| TF | ATF4 | Cryptic splicing, deletion in 5′UTR | CCGCCCACAGATGTAGTTTTC | CATCAAGTCCCCCACCAACACC |
| TF | BTF3 | Cryptic splicing, deletion in exon 1 | GCCCCTTATTCGCTCCGACAAG | TGTCATCTGCTGTGGCTGTTC |
| TF | Msx2 | Cryptic splicing, deletion in exon 2 | ACGCCCTTTACCACATCCCAGC | AAAGGTATACCGGAGGGAGGG |
| TF | NFIC | Exon skipping + alternative exon, | CCCTGGCGGCGATTACTACACT | TTCCTGGGACGATGGAGAAGGG |
| | | deletion in exon 7, exon 8 deleted, | TC | |
| | | alternative exon after exon 7 | | |
| TF | RELA | Cryptic splicing + exon skipping, | CCCAACACTGCCGAGCTCAAGA | CCAGAAGGAAACACCATGGTGGG |
| | | deletion in exon 7, exon 8 deleted, | TC | |
| | | deletion in exon 9 | | |
| TF | SNAI1 | Alternative exon + cryptic splicing, | CAATCGGAAGCCTAACTACAGC | CTCGGGGCATCTCAGACTCTAG |
| | | different 5′ exon, deletions in | | |
| | | exons 2 and 3 | | |
| TF | TFE3 | Cryptic splicing + exon skipping, | CCGAGGCAAAGGCCCTTTTGAA | AGAGCAGGGCAGGGTTCATG |
| | | deletion in exons 8 and 10, exon 9 | GG | |
| | | deleted | | |
| TF | TGIF | Cryptic splicing, alternative splice | TCCTTCGGCTGCGTTTCTGT | GGCAGAGAGAGAAAGGGACATCTT |
| | | donor in exon 1. | | |
| TF | Oct11a | Exon skipping, exon 10 spliced out | CTGGAGAAGTGGCTGAATGATG | TTTGGTCTCAGTGGAGGTAGGTG |
| | | | C | |
| TF | MAX | Alternative exon, alternative 3′exon | CAGTCCCATCACTCCAAGGA | as1 AGGTCCTTGGAGTGGAATGTG; |
| | | after exon 3. | | as2 AAAGGAGGCTGGAAGGTTGTAA |
| TF | PPARG | Alternative exon, alternative 5′ | s1 | CTTTATCTCCACAGACACGACAT |
| | | exon, does not change the protein | TGAAAGAAGCCGACACTAAACC | |
| | | | A; s2 | |
| | | | CATTTCTGCATTCTGCTTAATTC | |
| | | | CCT | |
| TF | BRD3 | Alternative exon, alternative 5′ and | s1 | as1 CATTAGCACTATGTCATCTGTG, |
| | | 3′ exons. | GTGCCCGCTTCTTCCATGCCGT | as2 TCCCGAGATTGGATGATGTGC |
| | | | CCT; s2 | |
| | | | ATGAGGTTTGCCAAGATGCCA | |
| TF | FoxH1 | Alternative exon + Intron retention, | CCTTTCCTCCAACCGATGCTTC | ATAGGCAAGTAGGAGGTGGGCAGC |
| | | different 5′UTR, retained intron | | |
| | | btween exons 3 and 4. | | |
| TF | SMARCC2 | Exon skipping, exon 11 spliced out | GACGGGCAAGGATGAGGATGA | TTTGTCAGGAAAGTTGAGCATTTGTT |
| | | | GA | GGG |
| TF | CBX3 | Cryptic splicing, cryptic splicing | CGTGTAGTGAATGGGAAAGTGG | TTTGCTTGGAATAATGGCATCTCAG |
| | | in exon 4 (D81bp), in-frame splicing | A | |
| | | altered protein. | | |
| TF | SMARCB1 | Cryptic splicing, cryptic splicing | GGCAGAAGCCCGTGAAGTTCC | TGGTCATCAAAGCAAAGGGAAAGGT |
| | | in exon IV, D27bp | AG | |
| TF | SMARCC1 | Exon skipping, exon 18 deleted | GACAGAGCAGACCAATCACATT | TACTCATAACTGGATTTCCTGACTGA |
| | | (D111bp) | A | C |
| TF | SMARCA5 | Exon skipping, exons 8, 9 and 10 | GAGATCTGTTTGTTTGATAGGA | GTTCTTTTAACTTAGGGAGCAGCT |
| | | deleted (D420bp) | GA | |
| TF | LISCH7 | Exon skipping, exon 4 spliced out | TGTATTACTGCTCCGTGGTCTC | TCTCCTCCCACCATTACTCGT |
| | | | AG | |
| TF | KLF5 | Alternative exon, additional exon | GTCCAGATAGACAAGCAGAGAT | AACCTCCAGTCGCAGCCTTC |
| | | after exon 3. | GC | |
| TF | CREB3L4 | Cryptic splicing, exon 2 uses a | ACAGAACAGGCATTCAGGAGTC | GAGCATAGGAGAACTGGTTGC |
| | | cryptic splice donor, leading to a | | |
| | | smaller exon. | | |
| TF | Hes6 | Exon skipping, exon 2 deleted | GACGGCTGGGCTGCTGCTGGG | GACTCAGTTCAGCCTCAGGG |
| TF | AR | Exon skipping, skipping of exon 2, | GGCCCCTGGATGGATAGCTACT | GCCTCATTCGGACACACTGGCTG |
| | | exon 3 and exon 4. | C | |
| TF | REST | Alternative exon, inclusion of an | GGCCCCATTCGCTGTGACCGCT | GGCCACATAACTGCACTGATCA |
| | | extra exon | | |

TABLE 3

Non-basal Transcription Modulator Splice Variants

| | | | | |
|---|---|---|---|---|
| Group | Symbol | Splicing Type | Sense | Asense |

| | | | | |
|---|---|---|---|---|
| Cytoskeletal | M-RIP | Exon skipping, exon 9 | GAGGTCTTATTGCGGGTAAAGG | GTGCTCAACTTGGATGGGACA |
| protein | | spliced out | | |
| Cytoskeletal | TAU | Alternative exon, exon | CCAAAATCAGGGGATCGCAGC | GGATGTTGCCTAATGAGCCAC |
| protein | | 10 inserted. | G | |
| Cytoskeletal | TNNT2 | Exon skipping, exons 4 | GAAGAGGTGGTGGAAGAGTAC | TCGGTCTCAGCCTCTGCTTCAG |
| protein | | and 5 deleted | G | |
| Growth | FGFR2 | Exon skipping + Alterna- | s1GGTTTACAGTGATGCCCAGC | as1CCCAATAGAATTACCCGCCAAGC; |
| factor/ | | tive exon, exons 2, 3 | C; | as2TGTTTTGGCAGGACAGTGAGC |
| Receptor | | deleted, alternative | s2GTGTGCAGATGGGATTAACG | |
| | | exon 5. | TC | |
| Growth | Her | Alternative exon, al- | s1GATGTACTGAGAATGTGCCC, | TCACCAGCTGGACATTCTCGG |
| factor/ | | ternative exon 7. | s2GAGTTTACTGGTGATCGCTG | |
| Receptor | | | CC | |
| Growth | NCAM | Alternative exon, exon | GGAGGACTTCTACCCGGAACAT | CAGTGTACTGGATGCTCTTCAGGG |
| factor/ | | insertion between exons | C | |
| Receptor | | 6 and 7. | | |
| Growth | VEGFR3 | Alternative exon, alter- | CAGATAGAGAGCAGGCATAGAC | as1TGAGGAGGAAAGGGCGTTTG; |
| factor/ | | native usage of the last | A | as2GTGCTGAAGGGACATTGTGAGAA |
| Receptor | | exon | | |
| Other | ADRM1 | Cryptic splicing, exon 3 | GACTCGCTTATTCACTTCTG | GTGGTGGATGACGGGGTGAC |
| | | differently spliced, | | |
| | | leading to a frameshift | | |
| Other | CD151 | Alternative exon, addi- | CGGACTCGGACGCGTGGTAG | CGCCACCACCAGGATGTAGG |
| | | tional exon after | | |
| | | exon II | | |
| Other | CD74 | Alternative exon, addi- | TGTTTGAAATGAGCAGGCACTC | GTTCCGACTTGGTTTGTCTTGT |
| | | tional exon after | | |
| | | exon 6. | | |
| Other | CHL1 | Exon skipping, exon 25 | GCTGGCACCTCTCAAACCTG | AGGCTTTTCATCACTGTCAC |
| | | deleted. | | |
| Other | CNTN4 | Exon skipping, exon 8 | AGGTCAAGGAATGGTGCTAC | TCTGGCTTTCCTTGCTATTG |
| | | deleted. | | |
| Other | CRK | Cryptic splicing, exon 2 | GCGTCTCCCACTACATCATCAA | CTAACACACAAGCCCTCCAGTTCGT |
| | | internal splicing | CAGC | |
| Other | DKFZp313H1 | Exon skipping, exons 13 | GCCTCAGACCAGAAAGTGAAG | GAAATCCATAGACCTTGTGGCG |
| | 733 | and 14 spliced out | | |
| Other | GT335 | Exon skipping, exon5 | GATGCGGAGTCTACGATGGGA | ACTTTCCAGTGAGTTCCAGC |
| | | skipping | C | |
| Other | HGD | Alternative exon, al- | TGAGTTACCTGACCTTGGACCA | TTCCTGGAGTTGGGAGTGAAGTG |
| | | ternative use of exons | | |
| | | 12 and 13. | | |
| Other | ISCU2 | Alternative exon, ad- | GGCCCGACTCTATCACAAG | TCCTTTCACCCATTCAGTGGC |
| | | ditional exon after 1 | | |
| | | exon | | |
| Other | KIAA1117 | Intron retention, Intron | CTCAGCAGTCTTAGTGGGTATC | GAGAATGGAGAGTTGGCACCTG |
| | | retained between exons | | |
| | | 12 and 13. | | |
| Other | LIV1 | Alternative exon, ad- | TGTTCGCGCCTGGTAGAGAT | TTTGGTTGATGATGGCTGGAC |
| | | ditional exon after | | |
| | | exon 1 | | |
| Other | LZ16 | Alternative exon, ad- | s1CTATGGAATCGCAGACGGTT | as1CACGCTCGTTTCTCTTGTTCACAT, |
| | | ditional exons after | GAT, | as2GCTCGTCGTCCTCATCAAACTCA |
| | | exons 2 and 3 | s2GCAAGAAGAAAGAGAAGCAG | |
| | | | GGC | |
| Other | MCAM | Cryptic splicing, new | GCCAACAGCACCTCCACAGA | AGCAGGGAGCTGGGAATGGT |
| | | splice acceptor in exon | | |
| | | 16, extended exon. | | |
| Other | MGC2747 | Cryptic splicing, cryp- | GCGATGAAACCAGGAACTCAC | GGAAGGCTGGTGTCTCTGTTA |
| | | tic splice site used in | | |
| | | exon 2. No protein. | | |
| Other | Nm23 | Exon skipping, exon 2 | CCTAAGCAGCTGGAAGGAACCA | GATTTCCTACAGCCTGGTCCTCT |
| | | spliced out. | T | |
| Other | NPIP | Cryptic splicing, al- | AGAGGAAGACCGCCAAAGAAC | GATAGAGCAGGCACTCGGCA |
| | | ternative splice ac- | ATC | |
| | | ceptor in exon 4. | | |
| Other | NYBR1 | Exon skipping + Alter- | AGTCCCTGTGAGACGGTTTC | ACTGTCTTTGTTGCTCCCTC |
| | | native exon, exon 17 | | |
| | | deleted, 6 additional | | |
| | | alternative exons after | | |
| | | exon 22. | | |
| Other | PEG1/MEST | Alternative exon, al- | s1GCATGGGATAACGCGGCCA; | AGAAGGAGTGGACGGTGAGT |
| | | ternative 5′ exon, not | s2CCTCAGGAAGCGCATGCG | |
| | | translated. | | |
| Other | PLP1 | Exon skipping, skipping | GCTTGTTAGAGTGCTGTGCA | GGAAACCAGTGTAGCTGCAG |
| | | of 5′ part of exon 3 | | |
| Other | PMSCL1 | Alternative exon, exon 9 | GTTGTTTCTACACCTGTGCTAT | GTATTATGGGAGCATCTGAGGTCA |
| | | inserted | GG | |
| Other | SELL | Exon skipping, exon 7 | GCTGCTCTGAAGGAACAAAC | GATAAATGAGGGGCGAAATG |
| | | spliced out | | |
| Other | SWAP70 | Exon skipping, exon 3 | CCACAGCGGCAAGGTCTCCAA | GCCTTTGCTAAACTGTCCATTTCCGA |
| | | deleted. | GT | |
| Other | TMPIT | cryptic splicing, 62 bp | GCCGCTTCCTGCTCAACTCCAG | GCCTCAATCCTTCTTGCTCC |
| | | skipped from the last | | |
| | | exon | | |
| Other | WBP2 | Cryptic splicing, alter- | CCCTGTTGGAGAGACTATGGCG | ATCCGCTGTCCGAACTCAATGG |
| | | native splice donorsite | | |
| | | in exon1. | | |
| RNA Binding | HNRNPB1 | Alternative exon, ad- | AAATCGGGCTGAAGCGACTGA | TTTGGCTCAACTACTCTCCCATC |
| Protein | | ditional exon after 1 | | |
| | | exon | | |
| RNA Binding | RNP6 | Alternative exon, al- | GAGTTCCAGGCTTCTGCCAA | TTCACCAAAGTATTGTTAATTAGCAG |
| Protein | | ternatively spliced exon | | |
| | | 5. | | |
| RNA Binding | SFRS5 | Intron retention, Intron | TTCATCGGGAGACTAAATCCAG | CCATAAGAGGCAAACTCAACCACC |
| Protein | | retained between exons 4 | CG | |
| | | and 5. | | |
| Signal | ALG8 | Exon skipping, exon 2 | GGGTGACTCTTCTCAAATGCCT | GCATTTACAGCACTCACGGAC |
| Transduction | | spliced out. | | |
| Signal | APBB1 | Cryptic splicing, al- | GCTCCCCAGAGGACACAGATTC | as1GCTCCCCAGAGGACACAGCCT |
| Transduction | | ternative splice accep- | | as2GCTCCTCCTCGGTCATCTCTAC |
| | | tor in exon 3 | | |
| Signal | Capn3 | Exon skipping, exon 15 | ATACCATCTCCGTGGATCGG | TTTGCCTTTGCCCTCCTCTGACT |
| Transduction | | spliced out | | |
| Signal | cdkn2a | Exon skipping | CTGCCCAACGCACCGAATAGTT | GAGCCTCTCTGGTTCTTTCAATCGG |
| Transduction | | | AC | |
| Signal | CSDA | Cryptic splicing, Al- | GTTCTCGCCACCAAAGTCCTTG | as1AGGAGGTCCCCTGCTTGGGC; |
| Transduction | | ternative splice accep- | | as2GGAGGTCCCCTGCTACGGTAC |
| | | tor in exon 7, leads to | | |
| | | 3 amino acid deletion | | |
| Signal | EAAT2 | Exon skipping, exon 8 | CGAAGAAAGTCCTGGTTGCAC | GGATACGCTGGGGAGTTTATTC |
| Transduction | | deleted. | | |
| Signal | GABARG2 | Exon skipping, exon 9 | CTGCTCTGGTGGAGTATGGCAC | TGCCGTCCAGACACTCATAGCC |
| Transduction | | spliced out | | |
| Signal | GLRA2 | Alternative exon, | TCTGCAAAGACCATGACTCC | AGCATGGATGGGTCCAAGTCC |
| Transduction | | alternative exon 3. | | |
| Signal | Hri | Exon skipping + cryptic | CCCACTTCGTTCAAGACAGG | ATCCAATCCCACAGCGAGAG |
| Transduction | | splicing, exons 4-8 | | |
| | | spliced out, exons 3 and | | |
| | | 9 use different splice | | |
| | | donor and acceptor. | | |
| Signal | ITGA4 | Alternative exon, ad- | CCTACACCTGAAAAACAAGA | GCTGTGTGACCCCAAACTGC |
| Transduction | | ditional exon after exon | | |
| | | 5 | | |
| Signal | ITGB4 | Alternative exon, al- | ACTACAACTCACTGACCCGCTC | TCCTCCATCCTGGGACTCTAT |
| Transduction | | ternative exon after | A | |
| | | exon 35 | | |
| Signal | ITPK1 | Alternative exon, 2 ad- | CTGAAAGGGAAGAGAGTTGGCT | TATCATTCTGGTCGGCTTCA |
| Transduction | | ditional exons after | | |
| | | exon 1 | | |
| Signal | Lyk5 | Alternative exon, 2 ad- | GGGCTGCTTGCTAACTCCA | ATGTGGCTGGCTTTGACACTC |
| Transduction | | ditional exons after | | |
| | | exon 2 | | |
| Signal | MAG | Alternative exon, al- | GCCATCGTCTGCTACATTACCC | AGCAGCCTCCTCTCAGATCC |
| Transduction | | ternative exon after | | |
| | | exon 10. | | |
| Signal | NMDAR1 | Exon skipping + cryptic | CCTACAAGCGGCACAAGGATG | CCGTGATATCAGTGGGATGG |
| Transduction | | splicing, exon 19 de- | | |
| | | leted, deletion in exon | | |
| | | 20 | | |
| Signal | PCF | Cryptic splicing, al- | TACTGGGAGGGCATTGACCA | TCCGAATGTCACGAACCTCCT |
| Transduction | | ternative splice accep- | | |
| | | tor inside exon 10 | | |
| Signal | pyridoxal | Cryptic splicing, al- | TTCAAACCACACAGGCTATGCC | ATGTCCATCACCCGCAAGGC |
| Transduction | kinase | ternative splice accep- | | |
| | | tor in exon 8. | | |
| Signal | RNF8 | Exon skipping, exon 7 | CAAATGGAGCAGGAACTTCAGG | TTCAGAGCAGCGGAGTCACG |
| Transduction | | spliced out | AC | |
| Signal | RPGR | Alternative exon, ad- | CCAGAGGAGAAGGAAGGAGCA | GGAACACTTTCATCATCTCCCACAG |
| Transduction | | ditional exon between | G | |
| | | exons 15 and 16 | | |
| Signal | SHMT1 | Alternative exon, ad- | GGCGGCGTAGGACGGAG | CGAGGCAATCAGCTCCAATC |
| Transduction | | ditional exon in 5′ UTR | | |
| | | after exon 1 | | |
| Signal | THTPA | Cryptic splicing, dele- | s1CTTGATTGAGGTGGAGCGAA | as1GCCTCTACCTCACCCACAGCGTA |
| Transduction | | tion in exon 1 | AGT | as2CTTGGCTGGTGCTGTCTCCTG |
| | | | s2GCACCGCACAACGGGCGTAA | |
| | | | TA | |
| Signal | Tyr | Exon skipping, exon 3 | GTGAGGACTAGAGGAAGAATG | GCCCTACTCTATTGCCTAAG |
| Transduction | | deleted. | C | |
| Signal | UBEC2C | Alternative exon, al- | GTGTTCTCCGAGTTCCTGTCTC | as1GGGAAGGGAGAAGTTGAGTCGG; |
| Transduction | | ternative 5′exon, if any | TC | as2CATTGTAAGGGTAGCCACTGGG |
| | | protein is translated, | | |
| | | the alternative Met is | | |
| | | used. | | |
| Signal | BAG4 | Exon skipping, exon 2 | GTACACCCACCTCCACCCTTAT | GCCACCAGTGACCATCCCAACAA |
| Transduction, | | spliced out. | ATCCT | |
| Death | | | | |
| Signal | Bcl6 | Cryptic splicing, exon 5 | ACCGCCAGCCTCTTATTCCAT | TTGTGGGATGGTGGAGTCCT |
| Transduction, | | spliced into two exons | | |
| Death | | | | |

TABLE 4

Non-basal Transcription Modulator Splice Variants

| | | | | |
|---|---|---|---|---|
| Group | Symbol | Splicing Type | Sense | Asense |

| | | | | |
|---|---|---|---|---|
| RNA Binding | HRNP | Exon skipping, exon 2 | TTCTCGAGCAGCGGCAGTTCTC | CACACAGTCTGTAAGCTTTCCC |
| Protein | | deleted | AC | |
| Other | BACS1 | Exon skipping, exons 9 | AATCAGGACCCACCTCTCTGCC | GGCTGGTTCTTTGGCTTCCTG |
| | | and 10 deleted | | |
| Other | CENPA | Exon skipping, exon 2 | TCCATCAACACGCTCTCGG | ACTGTCGTGCTTGCTCAGGA |
| | | skipping | | |
| Other | CD44 | Exon skipping, exons | CATCGGATTTGAGACCTGCAG | CTTCGACTGTTGACTGCAATGC |
| | | 6-11 deleted | | |
| Other | NEMP | Cryptic splicing, exon 6 | CCATGAAGCTGACGCGGAAGAT | as1 |
| | | cryptic splicing | GGT | CTCCTCCTCCGTCACAGCCTGGTT |
| | | | | as2 |
| | | | | GGGACAGGACTGGTGTAGACAGGCA |
| Other | EST | Alternative exon, ad- | GAGCGTGAGGCAGATCGGC | CCGAAACCACAAACCTTGCCAT |
| | | ditional exon spliced | | |
| | | in. | | |
| Signal | SUA1 | Alternative exon, ad- | GCAGGAGTGAAAGGACTGACC | GCCCATCTTCTACTCCTTGGCTAAC |
| Transduction | | ditional exon spliced in | | |
| | | after exon 3. | | |
| Signal | POMT1 | Cryptic splicing, ex- | CCGTGTTGTCCTACCTGAAGTT | GTAGGTGTCCTGGTGGGAATGAA |
| Transduction | | tended exon 8. | CT | |
| Other | galectin 9 | Exon skipping, exon 6 | CTTTGACCTCTGCTTCCTGGTG | TTGCGGACCACAGCATTCTCATC |
| | | spliced out. | C | |
| Signal | CA11 | Exon skipping + cryptic | GAAAGAGGAAAGACACAGAGA | TGGAGGATTCTGGCTCAGGA |
| Transduction | | splicing, exons 2-6 and | GAC | |
| | | the first half of exon 7 | | |
| | | spliced out. | | |
| Signal | GPX2 | Alternative exon, ad- | TCCTTCTATGACCTCAGTGCCA | ATGTTGATGGTTGGGAAGGTGCG |
| Transduction | | ditional exon after | TC | |
| | | exon 1. | | |
| Other | ccrg | Cryptic splicing, Inser- | GACGCTGTTCTTCCATCTTTACT | TTACCCAAGAATCAGGAATGGAAC |
| | | tion in 3′ UTR; doesn't | C | |
| | | affect protein | | |
| Other | SDCCAG1 | Exon skipping + alter- | GTTACAATGCTGCTAAGAGGAG | TCCAAACACAAGACTCATCTACC |
| | | native exon, one exon | GA | |
| | | skipped and one exon | | |
| | | inserted | | |
| Other | SDCCAG10 | Intron retention, intron | GGTAGTGTTTGGTGTCCCTGTC | GGTAGTGTTTGGTGTCCCTGTCT |
| | | retention in 5′UTR | T | |
| Other | SDCCAG8 | Alternative exon, exon 3 | GAACTGGATGAAAGCAAACAAC | CCTTAGCCTTTGCTTCATCGTCTC |
| | | insertion. Inserted | AC | |
| | | 192 bp. | | |
| Other | NY-BR-20 | Exon skipping + Alter- | CAAGGAATGCTTCTCCCTGTAT | GTTTGCCATCTCTCCCAAGTGAAA |
| | | native exon, exon 2 | GAC | |
| | | skipping, exon 3 inser- | | |
| | | tion. Alternative ATG. | | |
| Other | EPSTI1 | Alternative exon, two | TGGAAGACCAGAGAGAGGGTTT | CACTTCTGTCTGGCGATTCTGTG |
| | | additional exons spliced | G | |
| | | in. | | |
| Signal | PPP1R1B | Exon skipping, exons 1, | AGAGGCAGAGAGAGGAGACAC | CCTCATCTTCCTCTCTTGGATAACCC |
| Transduction | | 2, 5, 6 and 7 spliced | GCA | A |
| | | out | | |
| Other | USH1C | Exon skipping, exon 11 | GAAAAGTGGCCCGAGAATTCCG | TTCTCCTTTGCCGCTCCATCT |
| | | skipping | GCA | |
| Signal | CLIC5B | Alternative exon, alter- | s1 | as1 CTGAGAGAAAGGACAGTTGCC, |
| Transduction | | native 5′ exon. | GACGAAGACTACAGCACCATC, | as2 TGAACTCATCACGGGCATAGG |
| | | | s2 | |
| | | | AAGGAGTCGTGTTCAATGTCAC | |
| Other | Mic1 | Cryptic splicing, | ATCATCAGGATACAGAGACATC | GCAAGTGATTTCAGAATGTTGTAGGC |
| | | cryptic splicing in exon | GGTA | |
| | | IX | | |
| Other | PC-1 | Alternative exon, alter- | CCAAAGCGGCACTCAACTGAAG | CAGCCTGGGATAAGGTTTCAGATGTC |
| | | native exon I, ad- | G | |
| | | ditional exon between | | |
| | | exons 3 and 4 | | |
| RNA Binding | SF3B2 | Cryptic splicing, | GAGAGCCGCCAGGAAGAGATG | TCCTGGCTTCTTCTCCTTCAGTCG |
| Protein | | cryptic splicing in | AAT | |
| | | exons IX and X, D158bp | | |
| RNA Binding | DDX38 | Exon skipping, exons 3, | s1 | as1 AAACTCTTCGCTCACACCACCCG, |
| Protein | | 4, 5 and part of exon 6 | GCTTTCAAGGTGTGGATTTGGC | as2GCAAACTTCTCCGCATCCATCgtg |
| | | deleted (D746bp) | T; s2 | |
| | | | GGCACTGATCTGGACTGTCAGG | |
| | | | TT | |
| RNA Binding | DNAJC8 | Alternative exon, alter- | CAGCACCGAGGAAGCATTTATG | AATCTCTTCTTCCCTTTGTCGTTTCC |
| Protein | | native exon 2 | A | |
| RNA Binding | SFRS7 | Exon skipping, exon 7 | CTTGGCGGGTGAAGGTGTGTG | GGTTACACTTTACAGACATCACAAAT |
| Protein | | deleted | TCA | CCC |
| RNA Binding | SFRS9 | Cryptic splicing, exon 3 | GTGCGGATGTCGGGCTGGGCG | CTTGACCCAGACCGAGACCGTGAGT |
| Protein | | uses cryptic splice | GACGA | A |
| | | site. | | |
| RNA Binding | PRP19 | Exon skipping, exons | s1 | CCCTGCACAAGCCCTCCTGCCCAT |
| Protein | | 2-12 deleted, D1495bp | TGTCCCTAATCTGCTCCATCTCT, | |
| | | | s2 | |
| | | | GACCGACCAAATCCTGATAGTG | |
| | | | G | |
| Signal | RIPK2 | Exon skipping, exon 2 | ACCATGAACGGGGAGGCCATC | GTGAGAGGGACATCATGCGC |
| Transduction | | spliced out | TGC | |
| Other | neogenin1 | Exon skipping, exon 21 | AATCCAGGCACGGAACTCAA | GCGATAATCACAACCACCACG |
| | | spliced out | | |
| Other | ADRM1 | Cryptic splicing, exon 3 | ACCAGGATGAGGAGCATTGCC | ATCAGTGGGTGGGAGGTGAG |
| | | cryptic splicing | | |
| | | (D92 bp) | | |
| Signal | Bid | Exon skipping, exon 3 | GGGGCGC CATAAGGAGG | CTGGAACTGTCCGTTCAGTCCATC |
| Transduction | | deleted | AAGC | |
| Signal | Bax | Alternative exon, an | GATGGACGGGTCCGGGGAGCA | CTCAGCCCATCTTCTTCCAGATGGTG |
| Transduction, | | extra exon inserted be- | G | A |
| Death | | tween exons 4 and 5 | | |
| Signal | CASP9 | Exon skipping, skipping | GGCAGCTGATCATAGATCTGGA | CAGGGGAAGTGGAGGCCACCTC |
| Transduction, | | of exons 3, 4, 5, 6 | GAC | |
| Death | | | | |
| Signal | Bak | Alternative exon, an | GTGGGACGGCAGCTCGCCAT | GGCCATGCTGGTAGACGTGT |
| Transduction, | | extra exon between exons | | |
| Death | | 4 and 5 | | |
| Signal | BCL2L1 | Cryptic splicing, skip- | GCAACCGGGAGCTGGTGGTTG | CTGGTCATTTCCGACTGAAGAGT |
| Transduction, | | ping of 3′ part of exon | ACT | |
| Death | | 1 | | |
| Signal | Casp2 | Exon skipping + cryptic | GTGGAACTCCTCAACTTGCTG | GGTCAACCCCACGATCAGTCTCA |
| Transduction, | | splicing, skipping of | | |
| Death | | part of exon 3, exon 4 | | |
| | | entirely and part of | | |
| | | exon 5 | | |
| Other | SUMF2 | Exon skipping, exon 4 | GAGGCGACAGTGAAACCCTTTG | GTGCTCCAGTCTCTCTCGGATG |
| | | spliced out. | | |
| Other | G2AN | Exon skipping + cryptic | TTGGTCCTGATTCCCTCACGG | as1 CCCATATGCTACCAAGCGTGAG |
| | | splicing, exon 6 is | | as2 CTGGAAGGTAGGAGAGCTGTCTG |
| | | spliced out, exon 7 uses | | |
| | | different splice | | |
| | | acceptor. | | |
| Other | HCCR1 | Exon skipping, exons 3-6 | CCATCGTTTCTTGGGTCGTC | GGTAGTTGGTGGAGAGCAGG |
| | | spliced out. | | |
| Other | asns | Cryptic splicing, alter- | CAACAGTTCGTGCTTCAGTAGG | GGTGGCAGAGACAAGTAATAGG |
| | | native splice acceptor | | |
| | | in exon 4, leading to an | | |
| | | extended exon. | | |
| Signal | HSACP1 | Alternative exon, ad- | TCCGTGCTGTTTGTGTGTCTGG | GCTTTATGGGCTGTGTGAATGCC |
| Transduction | | ditional exon inserted | | |
| | | after exon 2. | | |
| Other | C20orf45 | Exon skipping, exon 3 | GTGTGGTTGGAGTTGATGTGTT | CTGCTGCCATTGGAGTCCTTATG |
| | | spliced out | GG | |
| Signal | macropain | Exon skipping, exons | GAAGCCAGTCCAGAGCCTAAG | AGCCAATGACAGGAAGTGTG |
| Transduction | | 6-17 spliced out. | G | |
| Signal | spi2 | Exon skipping, exon 2 | TGAGGAGCAGACCCAGGCAT | CTTCTGGGAGCACTTGGGACAG |
| Transduction | | deleted | | |
| Other | TCOF1 | Exon skipping, exon 21 | GACTCCTGGCATCAGAACCA | CCCTTCACCATCTTCCTCACTC |
| | | spliced out | | |
| Other | CIB1 | Intron retention, dif- | GGCGAGGACACACGGCTTAG | AACACAAACGGAGCAATGAC |
| | | ference in 3′UTR | | |
| | | (retained intron) | | |
| Other | TROAP | Intron retention, intron | s1 | as1 TCAGGCTGGTGGTTGCTGGA; |
| | | retained in the last | CCAGAGGAGTGCGGGGAACC; | as2 CGAACACCCTGGACCCTCTG |
| | | exon. | s2 | |
| | | | ACGCCTTTCCCCACTGTTAC | |
| Other | PARVA | Exon skipping, exon 8 | GATGTGTTGGTTGGAGAAAG | CTTGGATTTGCCGAGACTGG |
| | | skipping | | |
| Other | ILK | Alternative exon, ad- | s1 | as1 |
| | | ditional exon (exon 3a) | GCCTGGAGCCCGCCGAGAAC; | GCTGGGGATGTAGCCTGTCTG; |
| | | | s2 | as2 |
| | | | GGCGGCTTCTACATCACCTC | ACCACAGCATACAACTGCAC |
| Signal | ITGA7 | Intron retention, intron | GGTCCACGCCCGCTTCTGTA | TGACCTGGGCACCTCTCTTC |
| Transduction | | 16 retained. | | |
| Signal | ITGA5 | Exon skipping, exon 8 | TTGGGATTTGGGTCTTTTGT | GCAAGGCAAGGGATGGATAG |
| Transduction | | skipping | | |
| Growth factor/ | NCAM | Exon skipping, exons 17 | GAACGGAGGAGGAGAGGACC | TAGTGGTGACGGTGGTGACAG |
| Receptor | | and 18 deleted | | |
| Other | ZD52F10 | Alternative exon, alter- | ATGCGTATCCCACTGCCTATGG | AAGATGCTGGTGTATGTGACGAGG |
| | | native use of exon 2 | | |
| Signal | Diablo | Alternative exon + exon | CAATGGCGGCTCTGAAGAGTTG | CCTGGCGGTTATAGAGGCCT |
| Transduction, | | skipping, alternative | G | |
| Death | | exon 2 and exon 3 | | |
| | | skipping | | |
| Signal | CASP8 | Exon skipping + alter- | GGCAGGGCTCAAATTTCTGCCT | GATTGTTGATGATCAGACAGTATCC |
| Transduction, | | native exon, exon 4 and | ACA | |
| Death | | exon 8 skipping, exon 7 | | |
| | | inclusion | | |
| Signal | Casp3 | Exon skipping, exon2 | GTGCTATTGTGAGGCGGTTGTA | GACTGGATGAACCAGGAGCCA |
| Transduction, | | skipping, exon 7 | G | |
| Death | | skipping. | | |
| Signal | RON | Exon skipping, exon 5, | GGCTCCTGGCAACAGGACCAC | TTCTCCGTGGTAGACAACTCC |
| Transduction | | exon 6 and exon 11 | TG | |
| | | deleted. | | |
| Other | CD82 | Exon skipping, exon 9 | GCGTGGGGGCAGTCACTATGC | GGGGACCTTGCTGTAGTCTTCGGA |
| | | deleted | TCA | |
| Other | MUC2 | Cryptic splicing, skip- | CCCCTACTACCCCATGCGTGCC | GGTGTCGTTCAGGACACAGC |
| | | ping of 3′ part of Exon | TC | |
| | | 30 | | |
| Signal | RIOK1 | Cryptic splicing, | GGGCAATTCGACGACGCGGAC | CATTCTTGTTCTGGGATCCAAC |
| Transduction | | cryptic splicing of exon | T | |
| | | 3 | | |
| Other | RHAMM | Exon skipping, exon 4 | CTGGAGCTGGCCGTCAACATGT | CCAACTCAGTTTCCAGATCCTGG |
| | | spliced out | | |
| Other | DDR1a | Alternative exon + exon | GGGTCTGGCCAGGCTATGACTA | GAGGTCGCCGTTCTCCATGTAGTC |
| | | skipping, alternative 5′ | | |
| | | exons and skipping of | | |
| | | exon 11 | | |
| Growth factor/ | TNFRSF10B | Cryptic splicing, | CCCCAAGACCCTTGTGCTCGTT | GCAAAGTCATCGAAGCACTGTC |
| Receptor | | cryptic splicing in exon | GT | |
| | | 5 | | |
| Other | CSE1L | Alternative exon, an | CCCGAAGATGATACCATTCCTG | GCAGTGTCACACTGGCTGCC |
| | | extra exon (25 bp) in- | | |
| | | serted before last exon | | |
| Other | MLH1 | Exon skipping, exon 12 | CTACTCAGTGAAGAAGTGCAT | CGGGAATCATCTTCCACCAT |
| | | skipping | | |
| Other | MSH2 | Exon skipping, skipping | CCCAGGGGGTGATCAAGTACAT | GAGTGTCTGCATTGGTTCTACAT |
| | | of exons 2-8 | GG | |
| Signal | CCND1 | Exon skipping, G to A | GGAAGATCGTCGCCACCTGGAT | GGCATTTCCGTGGCACTAGGTGTCT |
| Transduction | | polymorphism in the end | | |
| | | of exon 4 results in | | |
| | | intron 4 retention and | | |
| | | exon 5 skipping | | |
| Growth factor/ | GHRHR | Exon skipping, skipping | CCTCTTTGTGAAGAGATGGCAC | GCCACTTCCGTGAGATCTCAGT |
| Receptor | | of exons 2, 3, 4 | C | |
| Signal | PTPN18 | Exon skipping, skipping | GCCGCTCTACAGCAAGGTGAC | CCTGGCTGTCCAGCTAGCAGAGA |
| Transduction | | of an exon in 3′ UTR, | | |
| | | protein sequence does | | |
| | | not change | | |
| Signal | ASC | Exon skipping, exon 2 | CCGCCGAGGAGCTCAAGAAGT | GGAGCAAGTCCTTGCAGGTCCA |
| Transduction | | skipping | TC | |
| Signal | BCL2L12 | Exon skipping, exon 6 | GGGTCTCCTGTTCCAACTCCAC | CCAATGGCAAGTTCAAGTCCAC |
| Transduction, | | skipping | CTA | |
| Death | | | | |
| Signal | NEK3 | Exon skipping, exon 14 | GCTCGGCTTGTCCAGAAGTGCT | CGGGGTTGTCATCTTCCTCCT |
| Transduction | | spliced out | TA | |
| Signal | Neu1 | Exon skipping, exon 2 | CCATGGGTAACAACTTCTCCAG | GGGCTAGGAGCTGCGGTAGGTCTTG |
| Transduction | | and 3 skipping (564 | TAT | |
| | | nucleotides) | | |

TABLE 5:
Non-basal Transcription Modulator Splice Variants

| SYMBOL | GENE ID | SPLICE TYPE |
|---|---|---|
| SRrp35 | 135295 | asv1, Exon 2 (107 nt) deleted, replaced |
| | | with new exon 2 (347 nt) just downstream |
| | | in the same intron; net change of +240 nt |
| SFRS14 | 10147 | asv1, Extra 93 nt exon between exons 10 and 11 |
| SFRS14 | 10147 | asv2, First: Extra 93 nt exon between exons |
| | | 10 and 11, Second: intron 9 looks unspliced but |
| | | clone is incomplete; Results in additional |
| | | 760 nts |
| PRPF8 | 10594 | asv1, Intron 31 unspliced, results in |
| | | 292 nt increase |
| PRPF8 | 10594 | asv2, intron 31 unspliced, exon 33 has deletion |
| SR-A1 | 58506 | asv1, 81 nt deletion in exon 6 |
| SR-A1 | 58506 | asv2, unspliced intron 3 (323 nt increase) |
| SFRS12 | 140890 | asv1, exon 9 missing |
| PRPF4 | 9128 | asv1, intron 4 unspliced |
| PRPF4 | 9128 | asv2, intron 11 unspliced |
| PRPF31 | 26121 | asv1, intron 12 unspliced |
| PRPF31 | 26121 | asv2, introns 10 and 12 unspliced |
| SF4 | 57794 | asv1, SF4; unique exon 5 |
| SFRS1 | 6426 | asv1, intron 3 unspliced |
| SFRS1 | 6426 | asv2, exon 1 extended 5′ |
| SRPK1 | 6732 | asv1, exon 10 missing |
| SFRS3 | 6428 | asv1, extra exon between exons 3 and 5 |

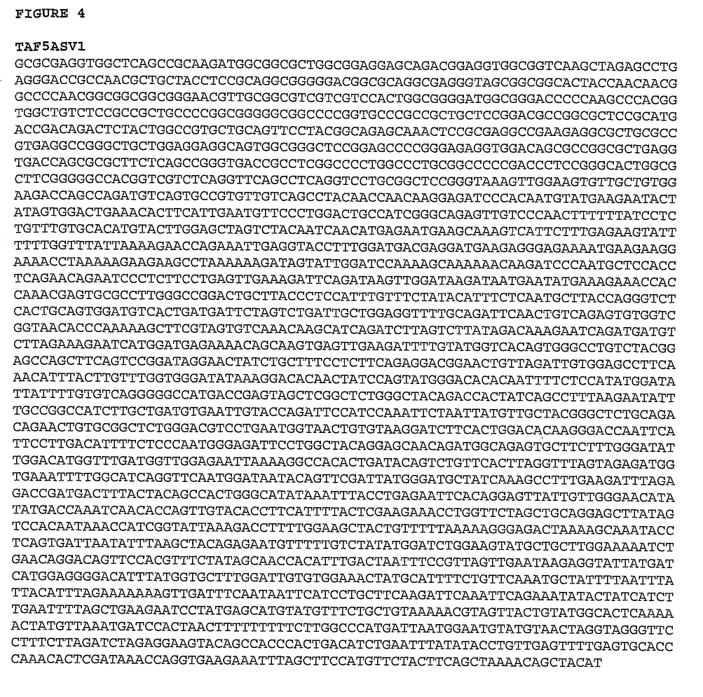

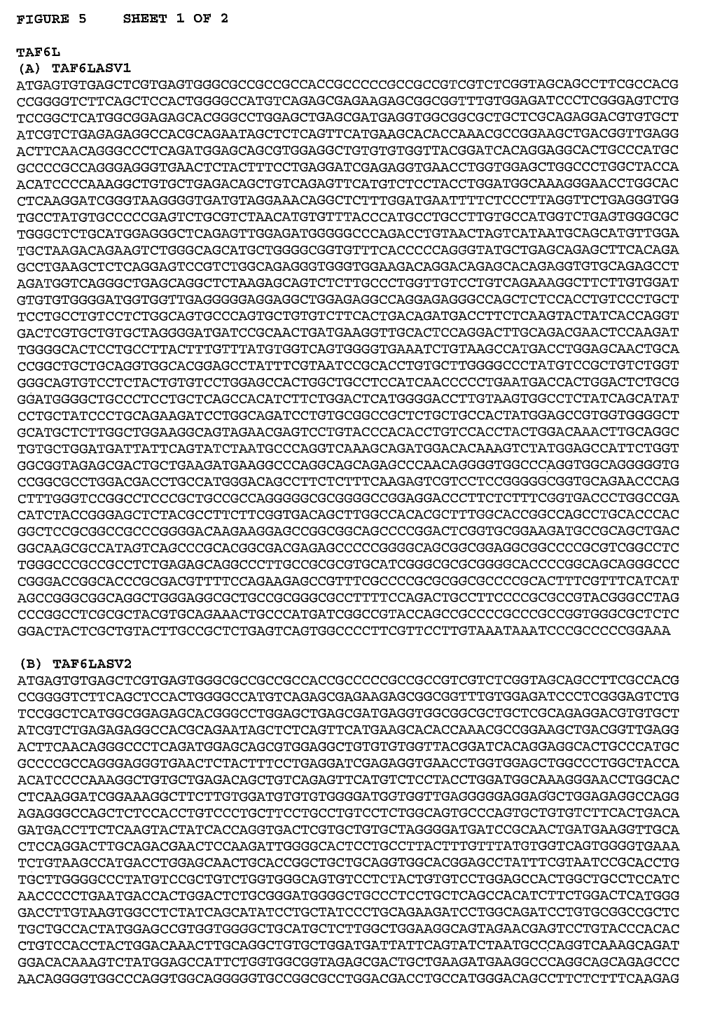

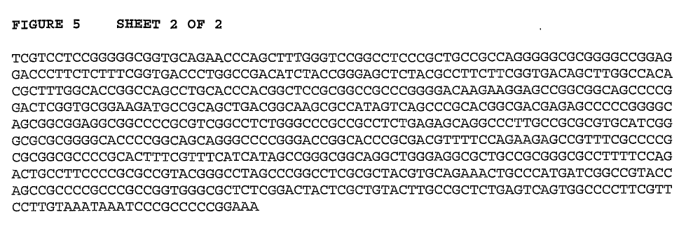

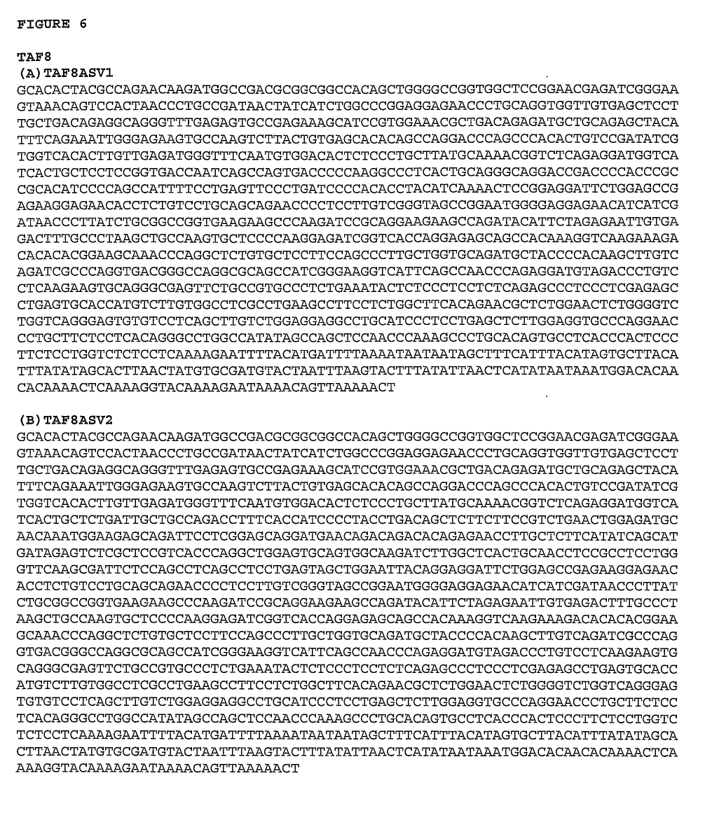

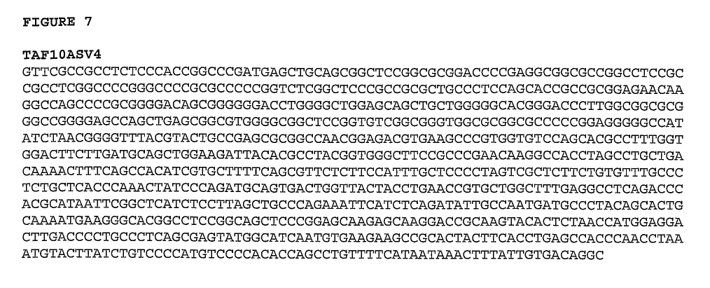

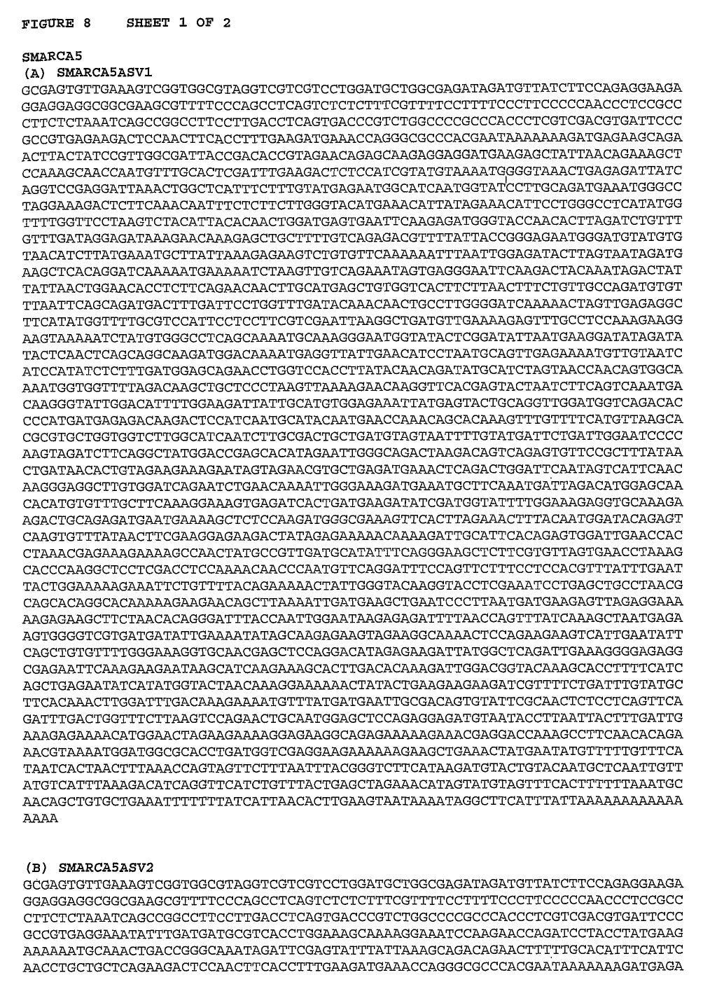

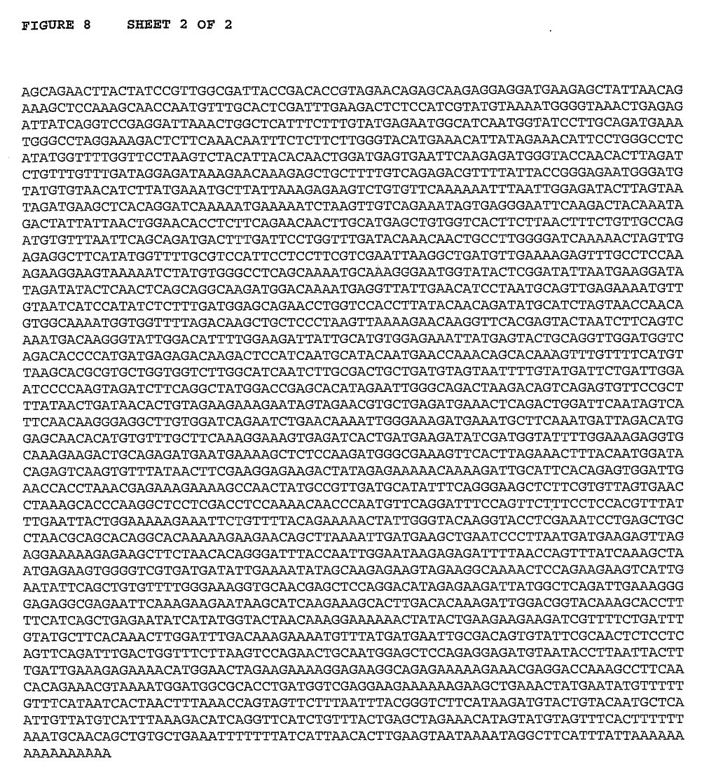

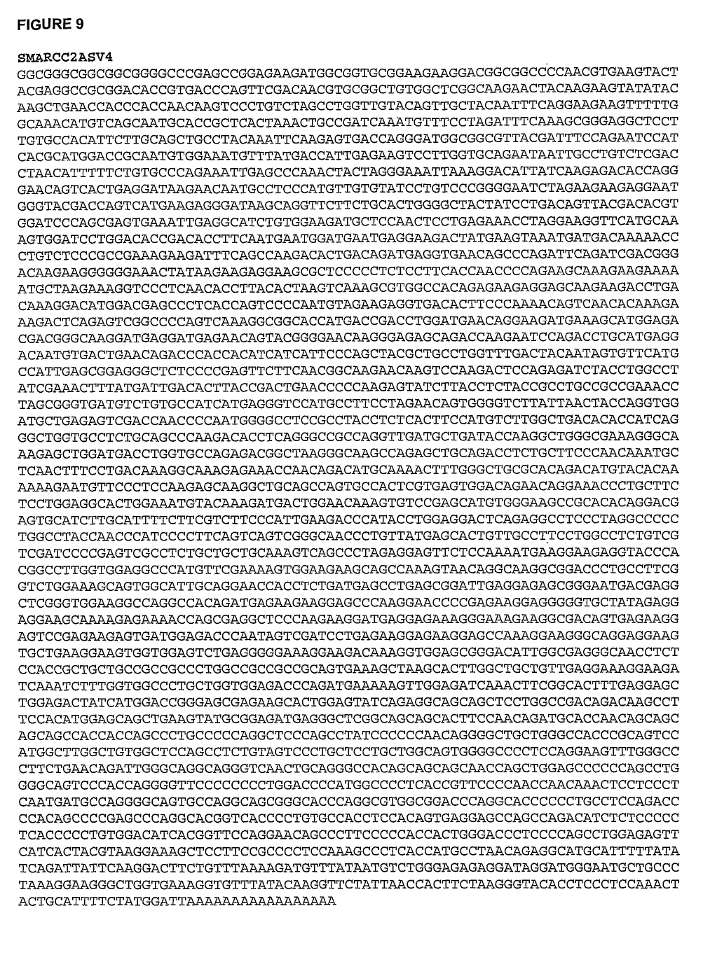

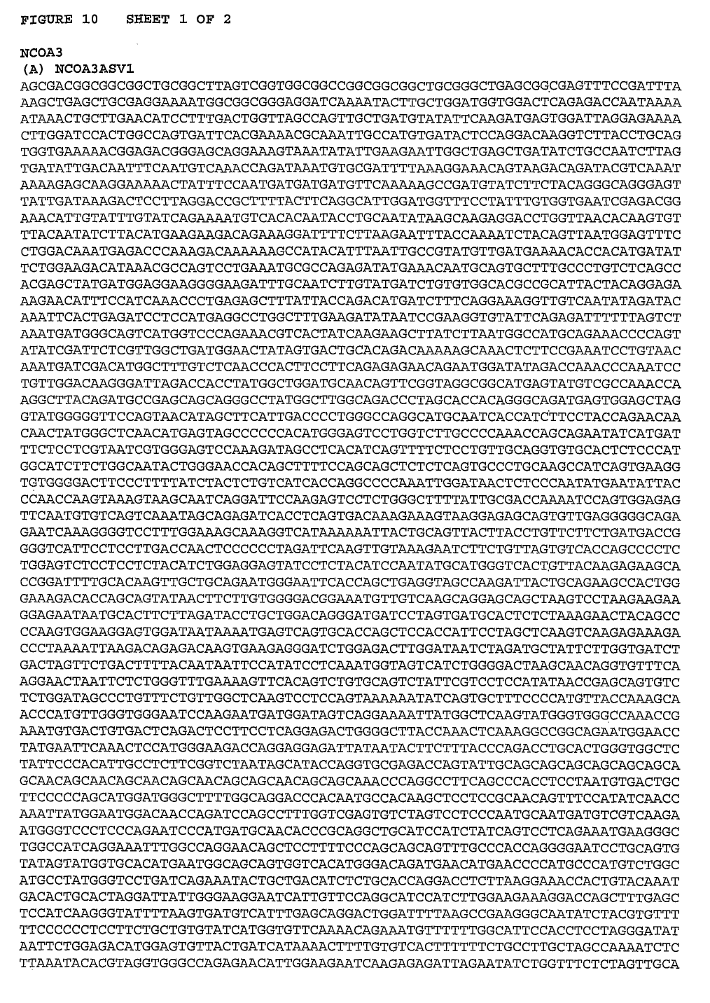

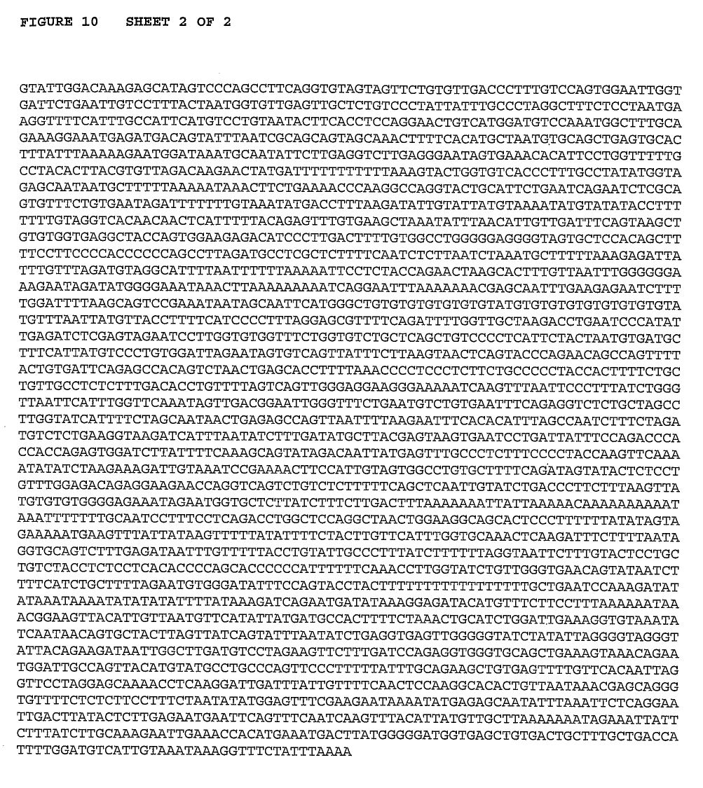

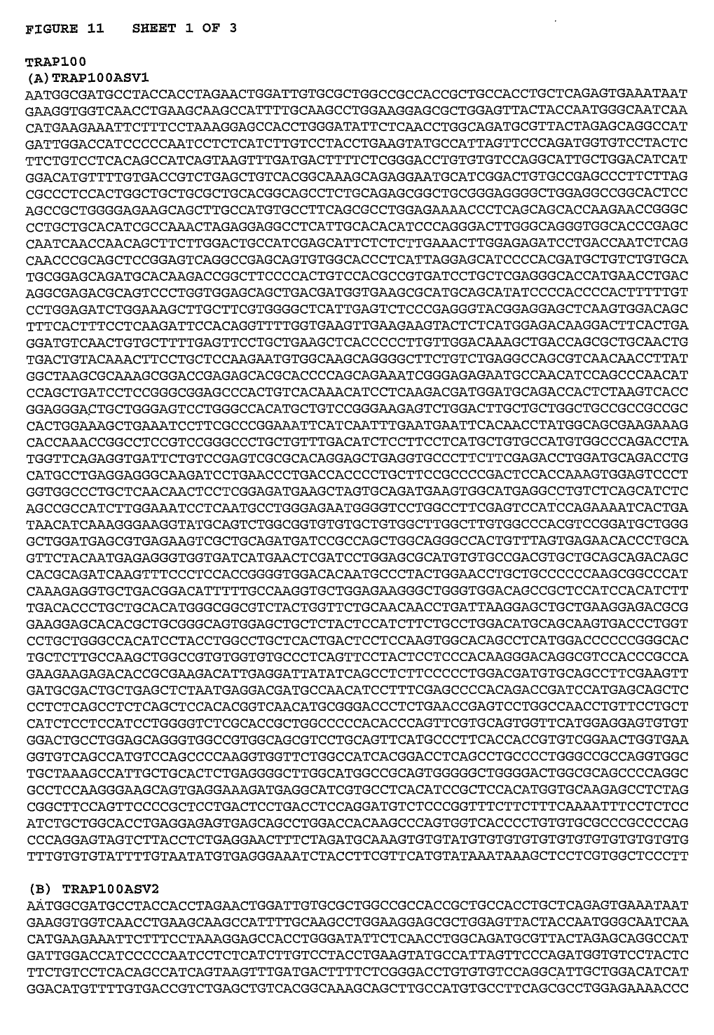

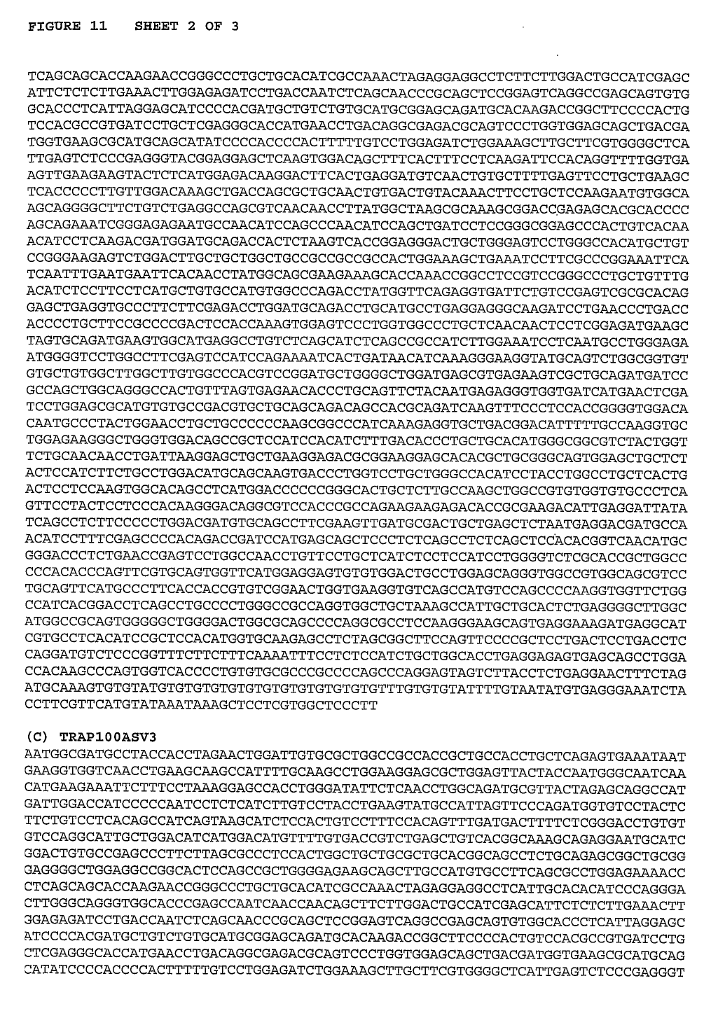

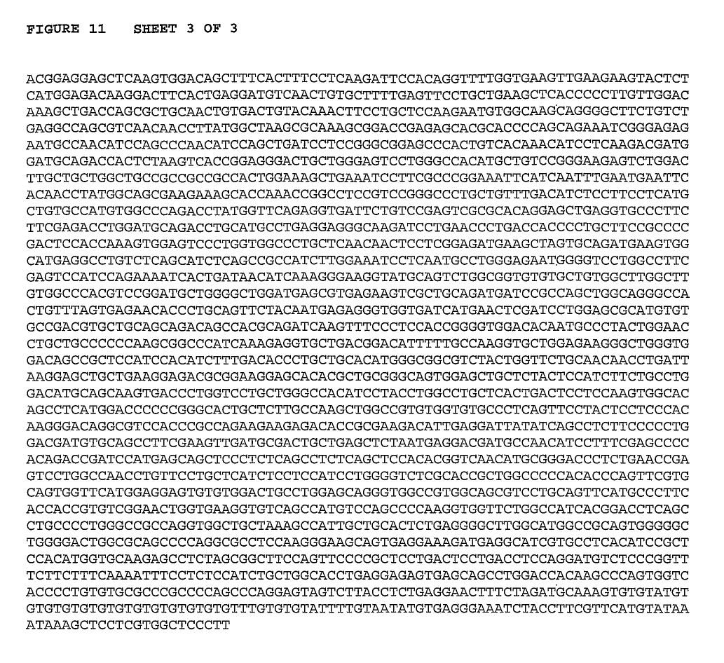

Also preferred are combinations of the primers provided herein with those disclosed in PCT/US03/41253 for the detection of tumor-specific/enriched splice variants of NRSF, MDM2, TSG, RREB, ZNF207, TTF-1, GTFIIIA, HES-6, HRY, Msx2, Neu, NeuroD1, Mash-1, and Irx2. Particularly preferred tumor-specific/enriched splice variants disclosed in PCT/US03/41253 are the novel tumor-specific/enriched splice variants of Neu, NeuroD1, Mash-1, and Irx2 disclosed in FIGS. 4-7 of PCT/US03/41253

Additionally, with respect to mRNA detection, oligonucleotide probes that hybridize to sequence not present in a wildtype transcript may be used to selectively detect expression of a splice variant of a transcription modulator. Such an approach is possible where alternative splicing generates a splice variant that contains a sequence insertion that is not present in the wildtype isoform of the transcription modulator. Such oligonucleotide probes are well suited for use in an array. An array may contain a plurality of such splice-variant specific oligonucleotide probes, and may contain probes for additional factors whose expression determination is of use in cancer diagnosis or prognosis, or provides relevant pharmacogenetic information, for example, how a patient will metabolize a particular drug.

The formation and use of nucleic acid arrays is well known in the art. For example, see Current Protocols in Molecular Biology, Greene Pub. Associates and Wiley-Interscience; New York; Eds. Ausubel et al., 1988/April 2003, Chapter 22, Nucleic Acid Arrays.

Preferred splice variants include those comprising the partial sequences set forth below. The partial sequences provided highlight the sequence variation in these preferred splice variants. It will be understood that minor sequence variations due to sequencing errors may be present.

TATA Associated Factors (TAFs)
wildtype TAF 2 = NM_003184
TAF2 ASV1
Novel exon (nt 1462-1627) following exon 9. Truncated protein of 522
amino acids long.
TTTGGTGTTAATGAGTACCGCCATTGGATTAAAGAGTGTCTTCCTTCTCAGGTGGAAGAATTGCAGCCTTTCATA
TCTTCATTAAACAAACCTTATCATCTTCCCCGTATTCTCATTTTACATATTATTATCATCCAAGAGTAAACTCAA
GTAAGCCAAAAAGTTAATTTTCGAAGACTTCAAACACCTAGAGCTATTAAGGAGCTAGACAAAATAGTGGCATAT
TATA Associated Factors (TAFs)
wildtype TAF 2 = NM_003184
TAF2 ASV2
Novel exon similar to ASV1 but 13 nucleotides shorter (1462-1614) after
exon 9. Truncated protein 408 amino acids long.
TTTGGTGTTAATGAGTACCGCCATTGGATTAAAGAGAGGTGGAAGAATTGCAGCCTTTCATATCTTCATTAAACA
AACCTTATCATCTTCCCCGTATTCTCATTTTACATATTATTATCATCCAAGAGTAAACTCAAGTAAGCCAAAAAG
TTAATTTTCGAAGACTTCAAACACCTAGAGCTATTAAGGAGCTAGACAAAATAGTGGCATATGAACTAAAAACTG
TATA Associated Factors (TAFs)
wildtype TAF4 = NM_003185
TAF4 ASV1 has exons 6-9 (nt. 1880-2480) spliced out. Truncated protein
628 amino acids long.
TTAATAAAACTGGCTTCATCTGGCAAGCAGTCTACAGAGACAGCAGCTAATGTGAAAGAGCTCGTGCAGAATTTA
CTGG-----------------------------------------------------------------
GACGATGATGACATTAATGATGTTGCATCGATGGCTGGAGTAAACTTGTCAGAAGAAAGTGCAAGAATATTAGCC
TATA Associated Factors (TAFs)
wildtype TAF4 = NM_003185
TAF4 ASV2, exon 7 (1969-2217) spliced out. Truncated protein 1000 amino
acids long (aa 656-739 out)
CTGGATGGAAAAATAGAAGCAGAAGATTTCACAAGCAGGTTATACCGAGAACTTAATTCTTCACCTCAACCTTAC
CTTGTGCCTTTCCTGAAG------------------------------------------------
GTCATCCAGCAGCCTCCGAAGCCAGGAGCCCTGATCCGGCCCCCGCAGGTGACGTTGACGCAGACACCCATGGTC
TATA Associated Factors (TAFs)
wildtype TAF4 = NM_003185
TAF4 ASV3, exons 6, 7 spliced out
see FIG. X
TATA Associated Factors (TAFs)
wildtype TAF4 = NM_003185
TAF4 ASV4, part of exon 7, 8 spliced out
see FIG. X
TATA Associated Factors (TAFs)
wildtype TAF4 = NM_003185
TAF4 ASV5, deletion in exon 1 (65-1355)
see FIG. X
TATA Associated Factors (TAFs)
wildtype TAF4 = NM_003185
TAF4 ASV6, combination of ASV2 and ASV5
TATA Associated Factors (TAFs)
wildtype TAF6L = NM_006473
TAF6 ASV1, unspliced intron between exons 5 and 6
see FIG. X
TATA Associated Factors (TAFs)
wildtype TAF6L = NM_006473
TAF6 ASV2, unspliced intron between exons 5 and 6
see FIG. X
TATA Associated Factors (TAFs)
wildtype TAF6L = NM_006473
TAF6 ASV3, Exon 1 extended 3′ by 116 nt
gagtgtgagctcgtgagtgggcgccgccgccaccgcccccgccgccgtcgtctcggtagcagccttcgccacgcc
ggggtcttcaggtgagcaggccttgctctggtccaaggactccccattcccgacgccgactgcttactcaccagt
cttggagcccgcaccgcgagggcccgcccccttggctgaccacgtgacccaactccactggggccatgtcagagc
gagaagagcggcggtttgtggagatccctcgggagtctgtccggctcatggcggagagcacgggcctggagctga
gcgatgaggtggcggcgctgctcgcagaggacgtgtgctatcgtctgagagaggccacgcagaatagctctcagt
tcatgaagcacaccaaacgccggaagctgacggttgaggacttcaacagggccctcagatggagcagcgtggagg
ctgtgtgtggttacggatcacaggaggcactgcccatgcgccccgccagggagggtgaactctactttcctgagg
atcgagaggtgaacctggtggagctggccctggctaccaacatccccaaaggctgtgctgagacagctgtcagag
ttcatgtctcctacctggatggcaaagggaacctggcacctcaaggatcggtgcccagtgctgtgtcttcactga
cagatgaccttctcaagtactatcaccaggtgactcgtgctgtgctaggggatgatccgcaactgatgaaggttg
cactccaggacttgcagacgaactccaagattggggcactcctgccttactttgtttatgtggtcagtggggtga
aatctgtaagccatgacctggagcaactgcaccggctgctgcaggtggcacggagcctatttcgtaatccgcacc
tgtgcttggggccctatgtccgctgtctggtgggcagtgtcctctactgtgtcctggagccac
TATA Associated Factors (TAFs)
wildtype TAF6L = NM_006473
TAF6 ASV4, Unspliced intron between exons 5 and 6, results in additional
533 nts
gagtgtgagctcgtgagtgggcgccgccgccaccgcccccgccgccgtcgtctcggtagcagccttcgccacgcc
ggggtcttcagctccactggggccatgtcagagcgagaagagcggcggtttgtggagatccctcgggagtctgtc
cggctcatggcggagagcacgggcctggagctgagcgatgaggtggcggcgctgctcgcagaggacgtgtgctat
cgtctgagagaggccacgcagaatagctctcagttcatgaagcacaccaaacgccggaagctgacggttgaggac
ttcaacagggccctcagatggagcagcgtggaggctgtgtgtggttacggatcacaggaggcactgcccatgcgc
cccgccagggagggtgaactctactttcctgaggatcgagaggtgaacctggtggagctggccctggctaccaac
atccccaaaggctgtgctgagacagctgtcagagttcatgtctcctacctggatggcaaagggaacctggcacct
caaggatcgggtaaggggtgatgtaggaaacaggctctttggatgaattttctcccttaggttctgagggtggtg
cctatgtgcccccgagtctgcgtctaacatgtgtttacccatgcctgccttgtgccatggtctgagtgggcgctg
ggctctgcatggagggctcagagttggagatgggggcccagacctgtaactagtcataatgcagcatgttggatg
ctaagacagaagtctgggcagcatgctggggcggtgtttcacccccagggtatgctgagcagagcttcacagagc
ctgaagctctcaggagtccgtctggcagagggtgggtggaagacaggacagagcacagaggtgtgcagagcctag
atggtcagggctgagcaggctctaagagcagtctcttgccctggttgtcctgtcagaaaggcttcttgtggatgt
gtgtggggatggtggttgagggggaggaggctggagaggccaggagagggccagctctccacctgtccctgcttc
ctgcctgtcctctggcagtgcccagtgctgtgtcttcactgacagatgaccttctcaagtactatcaccaggtga
ctcgtgctgtgctaggggatgatccgcaactgatgaaggttgcactccaggacttgcagacgaactccaagattg
gggcactcctgccttactttgtttatgtggtcagtggggtgaaatctgtaagccatgacctggagcaactgcacc
ggctgctgcaggtggcacggagcctatttcgtaatccgcacctgtgcttggggccctatgtccgctgtctggtgg
gcagtgtcctctactgtgtcctggagccac
TATA Associated Factors (TAFs)
wildtype TAF6L = NM_006473
TAF6 ASV5, Exons 6 and 7 spliced out, net loss of 169 nt
gagtgtgagctcgtgagtgggcgccgccgccaccgcccccgccgccgtcgtctcggtagcagccttcgccacgcc
ggggtcttcagctccactggggccatgtcagagcgagaagagcggcggtttgtggagatccctcgggagtctgtc
cggctcatggcggagagcacgggcctggagctgagcgatgaggtggcggcgctgctcgcagaggacgtgtgctat
cgtctgagagaggccacgcagaatagctctcagttcatgaagcacaccaaacgccggaagctgacggttgaggac
ttcaacagggccctcagatggagcagcgtggaggctgtgtgtggttacggatcacaggaggcactgcccatgcgc
cccgccagggagggtgaactctactttcctgaggatcgagaggtgaacctggtggagctggccctggctaccaac
atccccaaaggctgtgctgagacagctgtcagagttcatgtctcctacctggatggcaaagggaacctggcacct
caaggatcggggtgaaatctgtaagccatgacctggagcaactgcaccggctgctgcaggtggcacggagcctat
ttcgtaatccgcacctgtgcttggggccctatgtccgctgtctggtgggcagtgtcctctactgtgtcctggagc
cac
TATA Associated Factors (TAFs)
wildtype TAF6L = NM_006473
TAF6 ASV6, Exon 4 truncated on 3′ end, loss of 67 nt (alternate 5′ splice
site)
gagtgtgagctcgtgagtgggcgccgccgccaccgcccccgccgccgtcgtctcggtagcagccttcgccacgcc
ggggtcttcagctccactggggccatgtcagagcgagaagagcggcggtttgtggagatccctcgggagtctgtc
cggctcatggcggagagcacgggcctggagctgagcgatgaggtggcggcgctgctcgcagaggacgtgtgctat
cgtctgagagaggccacgcagaatagctctcagttcatgaagcacaccaaacgccggaagctgacggttgaggac
ttcaacagggccctcagatggagcagcgtggaggctgtgtgtggttacggatcacaggaggcactgcccatgcgc
cccgccagggagggtgaactctactttcctgaggatcgagagttcatgtctcctacctggatggcaaagggaacc
tggcacctcaaggatcggtgcccagtgctgtgtcttcactgacagatgaccttctcaagtactatcaccaggtga
ctcgtgctgtgctaggggatgatccgcaactgatgaaggttgcactccaggacttgcagacgaactccaagattg
gggcactcctgccttactttgtttatgtggtcagtggggtgaaatctgtaagccatgacctggagcaactgcacc
ggctgctgcaggtggcacggagcctatttcgtaatccgcacctgtgcttggggccctatgtccgctgtctggtgg
gcagtgtcctctactgtgtcctggagccac
TATA Associated Factors (TAFs)
wildtype TAF7L = NM_024885
TAF7L ASV1 a novel exon between exons 8 and 9 , new protein 375 amino acids
long.
ATTTTTGATATCCTCGGGAATGAGCAGCCACAAGCAGGGTCATACCTCGTCAGAATATGATATGCTTCGGGAGAT
GTTCAGTGATTCTAGAAGTAACAATGATGATGATGAGGATGAGGATGATGAAGATGAGGATGAGGATGAGGATGA
AGATGAAGACAAAGAAGAGGAGGAGGAAGATTGTTCTGAAGAGTATCTGGAAAGGCAGCTGCAGGCCGAGTTTAT
TGAATCTGGCCAGTATAGGGCAAATGAAGGTACCAGTTCAATAGTCATGGAAATTCAGAAGCAGATTGAGAAAAA
TATA Associated Factors (TAFs)
wildtype TAF8 = NM_138572
TAF8 ASV1, exons 6-8 spliced out
see FIG. X
TATA Associated Factors (TAFs)
wildtype TAF8 = NM_138572
TAF8 ASV2, different exons after 7, 9 is similar
see FIG. X
TATA Associated Factors (TAFs)
wildtype TAF8 = NM_138572
TAF8 ASV3, exons 5 and 6 spliced out
see FIG. X
TATA Associated Factors (TAFs)
wildtype TAF10 NM_006284
TAF10 ASV1 intronic sequence 3′ from exon 2 (unspliced, 413-622).
Truncated protein 138 amino acids long.
GGACTTCTTGATGCAGCTGGAAGATTACACGCCTACGGTGGGCTTCCGCCCGAACAAGGCCACCTAGCCTGCTGT
CAAAACTTTCAGCCACATCGTGCTTTTCAGCGTTCTCTTCCATTTGCTCCCCTAGTCGCTCTTCTGTGTTTGCCC
TCTGCTCACCCAAACTGTGAGCTTCCTGATAATCAGGCCTATCCATTTCCCTCACCCTCCTCCCGCTCTGCTGAC
AGTTCTCTTAATTGATTTCTCAGATCCCAGATGCAGTGACTGGTTACTACCTGAACCGTGCTGGCTTTGAGGCCT
TATA Associated Factors (TAFs)
wildtype TAF10 = NM_006284
TAF10 ASV2 intronic sequence 3′ from exon 4 (593-767) Truncated protein
190 amino acids long
CAATGATGCCCTACAGCACTGCAAAATGAAGGGCACGGCCTCCGGCAGCTCCCGGAGCAAGAGCAAGGTGTGAGG
GGAGGCTTAATGAATCAGTAATTACCTTCCACAACAGTGGAGGCTTATCCTGCCACCCCTTTCGGGAAACTGAAT
CGTAGGGGAGGTGTAAGACTTACTCAGGGTCACCCATCTGGGATTGAAGTCCGGGATTCCTGTGCTCAGTTGGTG
CTCTTCCCTCTTCCCTCAGGACCGCAAGTACACTCTAACCATGGAGGACTTGACCCCTGCCCTCAGCGAGTATGG
TATA Associated Factors (TAFs)
wildtype TAF10 = NM_006284
TAF10 ASV3 intronic sequences 3′ of exons 2 and 4
GGACTTCTTGATGCAGCTGGAAGATTACACGCCTACGGTGGGCTTCCGCCCGAACAAGGCCACCTAGCCTGCTGA
CAAAACTTTCAGCCACATCGTGCTTTTCAGCGTTCTCTTCCATTTGCTCCCCTAGTCGCTCTTCTGTGTTTGCCC
TCTGCTCACCCAAACTGTGAGCTTCCTGATAATCAGGCCTATCCATTTCCCTCACCCTCCTCCCGCTCTGCTGAC
AGTTCTCTTAATTGATTTCTCAGATCCCAGATGCAGTGACTGGTTACTACCTGAACCGTGCTGGCTTTGAGGCCT
CAGACCCACGCATGTGAGTAAACCCAGGGCAGGTTAGTTTTGGGTGCTTGTGCAGTATGTTGTCCATCTCCTTCT
CATCTAAGTTTTTTCTCTCTAGAATTCGGCTCATCTCCTTAGCTGCCCAGAAATTCATCTCAGATATTGCCAATG
TATA Associated Factors (TAFs)
wildtype TAF10 = NM_006284
TAF10 ASV4, intron after exon 2
see FIG. X
TATA Associated Factors (TAFs)
wildtype TAF10 = NM_006284
TAF10ASV5, Intron 2 unspliced (211 nt addition)
ggccatatctaacggggtttacgtactgccgagcgcggccaacggagacgtgaagcccgtggtgtccagcacgcc
tttggtggacttcttgatgcagctggaagattacacgcctacggtgggcttccgcccgaacaaggccacctagcc
tgctgtcaaaactttcagccacatcgtgcttttcagcgttctcttccatttgctcccctagtcgctcttctgtgt
ttgccctctgctcacccaaactgtgagcttcctgataatcaggcctatccatttccctcaccctcctcccgctct
gctgacagttctcttaattgatttctcagatcccagatgcagtgactggttactacctgaaccgtgctggctttg
aggcctcagacccacgcataattcggctcatctccttagctgcccagaaattcatctcagatattgccaatgatg
ccctacagcactgcaaaatgaagggcacggcctccggcagctcccggagcaagagcaaggaccgcaagtacactc
taaccatggaggacttgacccctgccctcagcgagtatggcatcaatgtgaagaagccgcactacttcacctgag
ccacccaacctaaatgtacttatctgtccccatgtccc
TATA Associated Factors (TAFs)
wildtype TAF15 = NM_139215
TAF15 ASV1, exon 15 spliced out, results in 485 amino acid protein that has
different COOH terminus.
GAAGGAATTCCTGCAATCAGTGCAATGAGCCTAGACCAGAGGACTCTCGTCCCTCAGGAGGA-------------
---------------------
GAAACGACTACAGAAATGATCAGCGCAACCGACCATACTGATGACTGTTTTGAATGTTCCTTTGTCTCTGACATG
TATA Associated Factors (TAFs)
wildtype TAF15 = NM_139215
TAF15 ASV2, Middle of exon 15 spliced out/deleted, loss of 465 nt
ttgatgaccctccttcagctaaggcagccattgactggtttgatggaaaagaattccatggcaacatcattaaag
tgtcctttgccactagaagacctgaattcatgagaggaggtggaagtggaggtgggcggcgaggccgtggaggat
atagaggtcgtggaggctttcaagggagaggtggagaccccaaaagtggggattgggtttgccctaatccgtcat
gcggaaatatgaactttgctcgaaggaattcctgcaatcagtgcaatgagcctagaccagaggactctcgtccct
caggaggagatttccgggggagaggctacggtggagagaggggctacagaggtcgtgggggcagaggtggagacc
gaggtggctatggaggcaaaatgggaggaagaaacgactacagaaatgatcagcgcaaccgaccatactgatgac
tgttttgaatgttcctttgtctctgacatgatccatagtgaaattgccagagttttgc
SMARC family (synonyms SWI/SNF; BAF; BRM; BRG)
wildtype SMARCA1 = NM_003069
SMARCA1 ASV1, exon 13 spliced out. Results in 1043 amino acid protein,
amino acids 543-554 are missing.
AGATTATTGCATGTGGCGTGGTTATGAGTATTGTCGACTGGATGGACAAACCCCGCATGAAGAAAGAGAG-----
---
GAGGAAGCAATAGAGGCTTTTAATGCTCCTAATAGTAGCAAATTCATCTTTATGCTAAGTACCAGGGCTGGAGGT
SMARC family (synonyms SWI/SNF; BAF; BRM; BRG)
wildtype SMARCA2 = NM_003070
SMARCA2 ASV1. Exon 29 (nt 4287-4339) spliced out. Protein 1568 amino
acids, lacks amino acids 1396-1412
CCCGCTGAGAAACTGTCACCAAATCCCCCCAAACTGACAAAGCAGATGAACGCTATCATCGATACTGTGATAAAC
TACAAAGATAG----------------------------------
TTCAGGGCGACAGCTCAGTGAAGTCTTCATTCAGTTACCTTCAAGGAAAGAATTACCAGAATACTATGAATTAAT
SMARC family (synonyms SWI/SNF; BAF; BRM; BRG)
wildtype SMARCA4 = NM_003072
SMARCA4 ASV1 Exon 27 is spliced out (nt 4051-4149). Protein 1614 amino
acids, lacks amino acids 1259-1290.
TTCGACCAGAAGTCCTCCAGCCATGAGCGGCGCGCCTTCCTGCAGGCCATCCTGGAGCACGAGGAGCAGGATGAG
------------------------------------------------------
GAGGAAGACGAGGTGCCCGACGACGAGACCGTCAACCAGATGATCGCCCGGCACGAGGAGGAGTTTGATCTGTTC
SMARC family (synonyms SWI/SNF; BAF; BRM; BRG)
wildtype SMARCA5 = NM_003601
SMARCA5 ASV1, exons 1-3 partially spliced out (222-794)
see FIG. X
SMARC family (synonyms SWI/SNF; BAF; BRM; BRG)
wildtype SMARCA5 = NM_003601
SMARCA5 ASV2, deletion in exon1 (nt 235-640)
see FIG. X
SMARC family (synonyms SWI/SNF; BAF; BRM; BRG)
wildtype SMARCA5 = NM_003601
SMARCA5 ASV3, alt. exon 1
gttttcccagcctcagtctctctttcgttttccttttcccttcccccaaccctccgcccttctctaaatcagccg
gccttccttgacctcagtgacccgtctggccccgcccaccctcgtcgacgtgattcccgccgtgaggaaatattt
gatgatgcgtcacctggaaagcaaaaggaaatccaagaaccagatcctacctatgaagaaaaaatgcaaactgac
cgggcaaatagattcgagtatttattaaagcagacagaactttttgcacatttcattcaacctgctgctcagaag
actccaacttcacctttgaagatgaaaccagggcgcccacgaataaaaaaagatgagaagcagaacttactatcc
gttggcgattaccgacaccgtagaacagagcaagaggaggatgaagagctattaacagaaagctccaaagcaacc
aatgtttgcactcgatttgaagactctccatcgtatgtaaaatggggtaaactgagagattatcaggtccgagga
ttaaactggctcatttctttgtatgagaatggcatcaatggtatccttgcagatgaaatgggcctaggaaagact
cttcaaacaatttctcttcttgggtacatgaaacattatagaaacattcctgggcctcatatggttttggttcct
aagtctacattacacaactggatgagtgaattcaagagatgggtaccaacacttagatctgtttgtttgatagga
gataaagaacaaagagctgcttttgtcagagacgttttattaccgggagaatggg
SMARC family (synonyms SWI/SNF; BAF; BRM; BRG)
wildtype SMARCB1 = NM_003073
SMARCB1 ASV1 deletion in exon 3 (nt 355-378). Protein 376 amino acids,
lacks amino acids 69-76)
AGGCGACTAGCCACTGTGGAAGAGAGGAAGAAAATAGTTGCATCGTCACATGAT------
CACGGATACACGACTCTAGCCACCAGTGTGACCCTGTTAAAAGCCTCGGAAGTGGAAGAGATTCTGGATGGCAAC
SMARC family (synonyms SWI/SNF; BAF; BRM; BRG)
wildtype SMARCC2 = NM_003075
SMARCC2 ASV1 deletion in exon 27 nt 3255-3600. Protein truncated at COOH
terminal end, 1099 amino acids, lacks amino acids 1075-1189.
TGCCAGGCAGCGGGCACCCAGGCGTGGCG----------------------------------------------
------------
GACCCAGGCACCCCCCTGCCTCCAGACCCCACAGCCCCGAGCCCAGGCACGGTCACCCCTGTGCCACCTCCACAG
SMARC family (synonyms SWI/SNF; BAF; BRM; BRG)
wildtype SMARCC2 = NM_003075
SMARCC2 ASV2 deletion in exon 27 (nt 3255-3531). Protein 1121 amino
acids, lacks amino acids 1075-1166.
TTCCCCCCCCTGGACCCCATGGCCCCTCACCGTTCCCCAACCAACAAACTCCTCCCTCAATGATGCCAGGGGCAG
TGCCAGGCAGCGGGCACCCAGGCGTGGCG----------------------------------------------
------------
GCCCAAAGCCCTGCCATTGTGGCAGCTGTTCAGGGCAACCTCCTGCCCAGTGCCAGCCCACTGCCAGACCCAGGC
SMARC family (synonyms SWI/SNF; BAF; BRM; BRG)
wildtype SMARCC2 = NM_003075
SMARCC2 ASV3 novel exon between exons 17 and 18 from nt 1682. Protein 1245
amino acids.
ATGCTGAGAGTCGACCAACCCCAATGGGGCCTCCGCCTACCTCTCACTTCCATGTCTTGGCTGACACACCATCAG
GGCTGGTGCCTCTGCAGCCCAAGACACCTCAGGGCCGCCAGGTTGATGCTGATACCAAGGCTGGGCGAAAGGGCA
AAGAGCTGGATGACCTGGTGCCAGAGACGGCTAAGGGCAAGCCAGAGCTGCAGACCTCTGCTTCCCAACAAATGC
TCAACTTTCCTGACAAAGGCAAAGAGAAACCAACAGACATGCAAAACTTTGGGCTGCGCACAGACATGTACACAA
SMARC family (synonyms SWI/SNF; BAF; BRM; BRG)
wildtype SMARCC2 = NM_003075
SMARCC2 ASV4, extra exon after 17 and deletion exon 27
see FIG. X
SMARC family (synonyms SWI/SNF; BAF; BRM; BRG)
wildtype SMARCC2 = NM_003075
SMARCC2 ASV5, deleted seq. in penultimate exon or extra exon after
penultimate exon, depending on context
cacttggctgctgttgaggaaaggaagatcaaatctttggtggccctgctggtggagacccagatgaaaaagttg
gagatcaaacttcggcactttgaggagctggagactatcatggaccgggagcgagaagcactggagtatcagagg
cagcagctcctggccgacagacaagccttccacatggagcagctgaagtatgcggagatgagggctcggcagcag
cacttccaacagatgcaccaacagcagcagcagccaccaccagccctgcccccaggctcccagcctatcccccca
acaggggctgctgggccacccgcagtccatggcttggctgtggctccagcctctgtagtccctgctcctgctggc
agtggggcccctccaggaagtttgggcccttctgaacagattgggcaggcagggtcaactgcagggccacagcag
cagcaaccagctggagccccccagcctggggcagtcccaccaggggttcccccccctggaccccatggcccctca
ccgttccccaaccaacaaactcctccctcaatgatgccaggggcagtgccaggcagcgggcacccaggcgtggcg
gcccaaagccctgccattgtggcagctgttcagggcaacctcctgcccagtgccagcccactgccagacccaggc
acccccctgcctccagaccccacagccccgagcccaggcacggtcacccctgtgccacctccacagtgaggagcc
agccagacatct
SMARC family (synonyms SWI/SNF; BAF; BRM; BRG)
wildtype SMARCD3 = NM_003078
SMARCD3 ASV1 exon 3 spliced out. Results in 22 amino acid short protein or
if reading frame shift then new 383 amino acids long protein
GCCCTTGGTGCTGCAGGCGCGGTGGGCTCCGGGCCCAGGCACCGAGGGGGCACTGGATGACTCTCCAGGTGCAGG
ACCCTGCCATCTATGACTCCAGGTCTTCAGCACCCACCCACCGTGGTACAG---------------
CAGTGCCAAGAGGAGGAAGATGGCTGACAAAATCCTCCCTCAAAGGATTCGGGAGCTGGTCCCCGAGTCCCAGGC
SMARC family (synonyms SWI/SNF; BAF; BRM; BRG)
wildtype SMARCD3 = NM_003078
SMARCD3 ASV2 exons 3, 4, 5 are spliced out (202-579). Protein 343 amino
acids
lacking amino acids 14-138
GCCCTTGGTGCTGCAGGCGCGGTGGGCTCCGGGCCCAGGCACCGAGGGGGCACTGGATGACTCTCCAGGTGCAGG
ACCCTGCCATCTATGACTCCAGGTCTTCAGCACCCACCCACCGTGGTACAG---------------
CAAAAGCGGAAGCTGCGACTCTATATCTCCAACACTTTTAACCCTGCGAAGCCTGATGCTGAGGATTCCGACGGC
NCOA family (SRC; NcoA)
wildtype NCOA2 = NM_006540
NCOA2 ASV1 exon 13 spliced out (nt 2768-2974). Protein 1385 amino acids,
lacks amino acids 868-937.
ACAGCTGAAAACAGCCCTGTCACACCTGTTGGAGCCCAGAAAACAGCACTGCGAATTTCACAGAGCA--------
---------------------------------
GAATGATTGGTAACAGTGCTTCTCGGCCTACTATGCCATCTGGAGAATGGGCACCGCAGAGTTCGGCTGTGAGAG
NCOA family (SRC; NcoA)
wildtype NCOA2 = NM_006540
NCOA2 ASV2, Exons 12 and 13 spliced out, results in loss of 418 nt
tagccagctctttgtcggatacaaacaaagactccacaggtagcttgcctggttctgggtctacacatggaacct
cgctcaaggagaagcataaaattttgcacagactcttgcaggacagcagttcccctgtggacttggccaagttaa
cagcagaagccacaggcaaagacctgagccaggagtccagcagcacagctcctggatcagaagtgactattaaac
aagagccggtgagccccaagaagaaagagaatgcactacttcgctatttgctagataaagatgatactaaagata
ttggtttaccagaaataacccccaaacttgagagactggacagtaagacagatcctgccagtaacacaaaattaa
tagcaatgaaaactgagaaggaggagatgagctttgagcctggtgaccaggaatgattggtaacagtgcttctcg
gcctactatgccatctggagaatgggcaccgcagagttcggctgtgagagtcacctgtgctgctaccaccagtgc
catgaaccggccagtccaaggaggtatgattcggaacccagcagccagcatccccatgaggcccagcagccagcc
tggccaaagacagacgcttcagtctcaggtcatgaatatagggccatctgaattagagatgaacatggggggacc
tcagtatagccaacaacaagctcctccaaatcagactgccccatggcctgaaagcatcctgcctatagaccaggc
gtcttttgccagccaaaacaggcagccatttggcagttctccagatgacttgctatgtccacatcctgcagctga
gtctccgagtgatgagggagctctcct
NCOA family (SRC; NcoA)
wildtype NCOA2 = NM_006540
NCOA2 ASV3, Deletion from early in exon 12 to late in exon 14, exon 13
completely deleted, net loss of 442 nt
tagccagctctttgtcggatacaaacaaagactccacaggtagcttgcctggttctgggtctacacatggaacct
cgctcaaggagaagcataaaattttgcacagactcttgcaggacagcagttcccctgtggacttggccaagttaa
cagcagaagccacaggcaaagacctgagccaggagtccagcagcacagctcctggatcagaagtgactattaaac
aagagccggtgagccccaagaagaaagagaatgcactacttcgctatttgctagataaagatgatactaaagata
ttggtttaccagaaataacccccaaacttgagagactggacagtaagacagatcctgccagtaacacaaaattaa
tagcaatgaaaactgagaaggaggagatgagctttgagcctggtgaccagcctggcagtgagctggacaacttgg
aggagattttggatgatttgcagaagtcacctgtgctgctaccaccagtgccatgaaccggccagtccaaggagg
tatgattcggaacccagcagccagcatccccatgaggcccagcagccagcctggccaaagacagacgcttcagtc
tcaggtcatgaatatagggccatctgaattagagatgaacatggggggacctcagtatagccaacaacaagctcc
tccaaatcagactgccccatggcctgaaagcatcctgcctatagaccaggcgtcttttgccagccaaaacaggca
gccatttggcagttctccagatgacttgctatgtccacatcctgcagctgagtctccgagtgatgagggagctct
cct
NCOA family (SRC; NcoA)
wildtype NCOA3 = NM_181659
NCOA3 ASV1, 3145-(3950-3980) out in stretch of CAG
see FIG. X
NCOA family (SRC; NcoA)
wildtype NCOA4 = NM_005437
NCOA4 ASV1 exon 8 is spliced out (nt. 855-1838). Protein 286 amino acids
lacks amino acids 239-565.
GGCTCCTTGGAAGCAAACCTGCCAGTGGTTATCAAGCTCCTTACATACCCAGCACCGACCCCCAGGACTGGCTTA
CCCAAAAGCAGACCTTGGAGAACAGTCAG----------
GAAGTATTACTTAATTCACCTCTACAGGAGGAACATAACTTCCCCCCAGACCATTATGGCCTCCCTGCAGTTTGT
NCOA family (SRC; NcoA)
wildtype NCOA6 = NM_014071
NCOA6 ASV1 part of exon 8 is spliced out, nt 1851-1882. Truncated protein
568 amino acids.
GCAGCCTGTCAGCTCTCCGGGTCGGAATCCTATGGTTCAACAGGGAAATGTGCCACCTAACTTCATGGTGATGCA
GCAGCAACCACCAAACCAGGGGCCACAGAGTTTACATCCAGGCCTAGGAG----------------------
AGCAGGACAGGCCAATCCGAACTTTATGCAAGGTCAGGTGCCTTCGACCACAGCAACCACCCCTGGGAATTCAGG
NCOA family (SRC; NcoA)
wildtype NCOA7 = NM_181782
NCOA7 ASV1 exon 3 spliced out (nt 215-435). Protein 869 amino acids
TTTGATTGTGTATTATGGATACCAAGGAAGAGAAGAAGGAACGGAAACAAAGTTATTTTGCTCG--
AGATGACAATCAAAACAAAACACATGATAAAAAAGAGAAGAAGATGGTGGTTCAGAAGCCCCATGGGACTATGGA
TRAP100
wildtype = NM_014815
ASV1, new exon between exons 6 and 7
see FIG. X
TRAP100
wildtype = NM_014815
ASV2, splicing inside exon 6
see FIG. X
TRAP100
wildtype = NM_014815
ASV3, new exon after 4, and between 6 and 7
see FIG. X
MED12
gene id: 9968
asv1, introns 8, 11 unspliced
cagcaatctctgagaccaaggttaagaagagacatgttgaccctttcatggaatggactcagatcatcaccaagt
acttatgggagcagttacagaagatggctgaatactaccggccagggcctgcaggaagtgggggctgtggttcca
cgatagggcccttgccccatgatgtagaggtggcaatccggcagtgggattacaccgagaagctggccatgttca
tgtttcaggatggaatgctggacagacatgagttcctgacctgggtgcttgagtgttttgagaagatccgccctg
gagaggatgaattgcttaaactgctgctgcctctgcttctccgatactctggggaatttgttcagtctgcatacc
tgtcccgccggcttgcctacttctgtacacggagactggccctgcagctggatggtgtgagcagtcactcatctc
atgttatatctgctcagtcaacaagcacgctacccaccacccctgctcctcagcccccaactagcagcacaccct
cgactccctttagtgacctgcttatgtgccctcagcaccggcccctggtttttggcctcagctgtatcctacaga
ccatcctcctgtgctgtcctagtgccttggtttggcactactcactgactgatagcagaattaagaccggctcac
cacttgaccacttgcctattgccccgtccaacctgcccatgccagagggtaacagtgccttcactcagcaggtat
gtctgaccactagcctggtactctcagattgggctatgaggctaaattactctttcagaagtagtgatttggagt
ctagtactattcttctagcctggggctctggccttttatatgccttggtacatccttgtagccttcctttttaac
attgcaggtccgtgcaaagttgcgggagatcgagcagcagatcaaggagcggggacaggcagttgaagttcgctg
gtctttcgataaatgccaggaagctactgcaggcttcaccattggacgggtacttcatactttggaagtgctgga
cagccatagttttgaacgctctgacttcagcaactctcttgactccctttgtaaccgaatctttggattgggacc
tagcaaggatgggcatgagatctcctcagatgatgatgctgtggtgtcattgctatgtgaatgggctgtcagctg
caagcgttctggtcggcatcgtgctatggtggtagccaagctcctggagaagagacaggcggagattgaggctga
ggttagagggcagagataagagaacaagattggccaatgggaaggaatttactgcggttggagaccgagagatgg
aggtggtggagggaccagagttgaaggtgtgagaacagagtaaagaagcaaaagagaacctaaaggcaaagttac
ggacgtgaggcgaaagtagagaagagtggattgtagtaagagttagagataacatcaaggcttcagttgggaggt
ggtaaagaacatggaggtcagcaggggaatgaaagtgaaaagcatggggtagaggtcaagcaggtggtagtttaa
ggcctacacattgaggagtgaagaagcaggtaaaagtcagttctacaatttgttctgtcatcttgcagcgttgtg
gagaatcagaagccgcagatgagaagggttccatcgcctctggctccctttctgctcccagtgctcccattttcc
aggatgtcctcctgcagtttctg
MED12
gene id: 9968
asv2, intron 18 unspliced
cagcaatctctgagaccaaggttaagaagagacatgttgaccctttcatggaatggactcagatcatcaccaagt
acttatgggagcagttacagaagatggctgaatactaccggccagggcctgcaggaagtgggggctgtggttcca
cgatagggcccttgccccatgatgtagaggtggcaatccggcagtgggattacaccgagaagctggccatgttca
tgtttcaggatggaatgctggacagacatgagttcctgacctgggtgcttgagtgttttgagaagatccgccctg
gagaggatgaattgcttaaactgctgctgcctctgcttctccgatactctggggaatttgttcagtctgcatacc
tgtcccgccggcttgcctacttctgtacacggagactggccctgcagctggatggtgtgagcagtcactcatctc
atgttatatctgctcagtcaacaagcacgctacccaccacccctgctcctcagcccccaactagcagcacaccct
cgactccctttagtgacctgcttatgtgccctcagcaccggcccctggtttttggcctcagctgtatcctacaga
ccatcctcctgtgctgtcctagtgccttggtttggcactactcactgactgatagcagaattaagaccggctcac
cacttgaccacttgcctattgccccgtccaacctgcccatgccagagggtaacagtgccttcactcagcaggtcc
gtgcaaagttgcgggagatcgagcagcagatcaaggagcggggacaggcagttgaagttcgctggtctttcgata
aatgccaggaagctactgcaggcttcaccattggacgggtacttcatactttggaagtgctggacagccatagtt
ttgaacgctctgacttcagcaactctcttgactccctttgtaaccgaatctttggattgggacctagcaaggatg
ggcatgagatctcctcagatgatgatgctgtggtgtcattgctatgtgaatgggctgtcagctgcaagcgttctg
gtcggcatcgtgctatggtggtagccaagctcctggagaagagacaggcggagattgaggctgagcgttgtggag
aatcagaagccgcagatgagaagggttccatcgcctctggctccctttctgctcccagtgctcccattttccagg
atgtcctcctgcagtttctggatacacaggctcccatgctgacggaccctcgaagtgagagtgagcgggtggaat
tctttaacttagtactgctgttctgtgaactgattcgacatgatgttttctcccacaacatgtatacttgcactc
tcatctcccgaggggaccttgcctttggagcccctggtccccggcctccctctccctttgatgatcctgccgatg
acccagagcacaaggaggctgaaggcagcagcagcagcaagctggaagatccagggctctcagaatctatggaca
ttgaccctagttccagtgttctctttgaggacatggagaagcctgatttctcattgttctcccctactatgccct
gtgaggggaagggcagtccatcccctgagaagccagatgtcgagaaggaggtgaagcccccacccaaggagaaga
ttgaagggacccttggggttctttacgaccagccacgacacgtgcagtacgccacccattttcccatcccccagg
aggagtcatgcagccatgagtgcaaccagcggttggtcgtactgtttggggtgggaaagcagcgagatgatgccc
gccatgccatcaagaaaatcaccaaggatatcttgaaggttctgaaccgcaaagggacagcagaaactgaccagc
ttgctcctattgtgcctctgaatcctggagacctgacattcttaggtggggaggatgggcagaagcggcgacgca
accggcctgaagccttccccactgctgaagatatctttgctaagttccagcacctttcacattatgaccaacacc
aggtcacggctcaggtgtgggcctaagcccagcccctttcccacattctggcctcctgttctgttttccttttct
tccctatcttctccctgctaggcaggctaagcctcctggtctcatccccttccagtgtcatcctttcctccttcc
ctggttctttcctctctccactcccatctcactcccactgcccttatcaggtctcccggaatgttctggagcaga
tcacgagctttgcccttggcatgtcataccacttgcctctggtgcagcatgtgcagttcatcttcgacctcatgg
a
MED12
gene id: 9968
asv3, Deletion from mid-exon 11 through mid-exon 19
tgatgatgctgtggtgtcattgctatgtgaatgggctgtcagctgcaagcgttctggtcggcatcgtgctatggt
ggtagccaagctcctctggtgcagcatgtgcagttcatcttcgacctcatgga
MED12
gene id: 9968
asv4, Intron 21 unspliced AND exon 22 truncated on 3′end by 31 nt (net
increase of 394 nt)
tgatgatgctgtggtgtcattgctatgtgaatgggctgtcagctgcaagcgttctggtcggcatcgtgctatggt
ggtagccaagctcctggagaagagacaggcggagattgaggctgagcgttgtggagaatcagaagccgcagatga
gaagggttccatcgcctctggctccctttctgctcccagtgctcccattttccaggatgtcctcctgcagtttct
ggatacacaggctcccatgctgacggaccctcgaagtgagagtgagcgggtggaattctttaacttagtactgct
gttctgtgaactgattcgacatgatgttttctcccacaacatgtatacttgcactctcatctcccgaggggacct
tgcctttggagcccctggtccccggcctccctctccctttgatgatcctgccgatgacccagagcacaaggaggc
tgaaggcagcagcagcagcaagctggaagatccagggctctcagaatctatggacattgaccctagttccagtgt
tctctttgaggacatggagaagcctgatttctcattgttctcccctactatgccctgtgaggggaagggcagtcc
atcccctgagaagccagatgtcgagaaggaggtgaagcccccacccaaggagaagattgaagggacccttggggt
tctttacgaccagccacgacacgtgcagtacgccacccattttcccatcccccaggaggagtcatgcagccatga
gtgcaaccagcggttggtcgtactgtttggggtgggaaagcagcgagatgatgcccgccatgccatcaagaaaat
caccaaggatatcttgaaggttctgaaccgcaaagggacagcagaaactgaccagcttgctcctattgtgcctct
gaatcctggagacctgacattcttaggtggggaggatgggcagaagcggcgacgcaaccggcctgaagccttccc
cactgctgaagatatctttgctaagttccagcacctttcacattatgaccaacaccaggtcacggctcaggtctc
ccggaatgttctggagcagatcacgagctttgcccttggcatgtcataccacttgcctctggtgcagcatgtgca
gttcatcttcgacctcatggaatattcactcagcatcagtggcctcatcgactttgccattcagctgctgaatga
actgagtgtagttgaggctgagctgcttctcaaatcctcggatctggtgggcagctacactactagcctgtgcct
gtgcatcgtggctgtcctgcggcactatcatgcctgcctcatcctcaaccaggaccagatggcacaggtctttga
ggggctgtgtggcgtcgtgaagcatgggatgaaccggtccgatggctcctctgcagagcgctgtatccttgctta
tctctatgatctgtacacctcctgtagccatttaaagaacaaatttggggagctcttcaggtaagagaggtggaa
ggtaaggggtagcgagtgggacctactcccttcttcccatgaccacccaactcaggaggagaggatggcccggga
ccctgctgcctgtctagggtcatttgtggactgtgtcctccacatactgttgtgttaccaagagtgggccctctt
cctcagcaggcttgctccccgcctatatctgtggggcccaccctcttcccccttttcctcactgccttcagaggc
cccagttccttattcccatgtggttcctttcctgcccagtctgttttgtcccatctcccttttcttgtctcaaga
tccttcatccctcactttctcctttttttcttttctcccctttcctgaccatccctcgacctcagcaggccttct
tcaacactactatctcctttcctccatccctgcagcgacttttgctcaaaggtgaagaacaccatctactgcaac
gtggagccatcggaatcaaatatgcgctgggcacctgagttcatgatcgacactctagagaaccctgcagctcac
accttcacctacacggggctagtagggtgaatgacatcgcaatcctgtgtgcagagctgaccggctattgcaagt
cactgagtgcagaatggctaggagtgcttaaggccttgtgctgctcctctaacaatggcacttgtggtttcaacg
atctcctctgcaatgttgatgtcagtgacctatcttttcatgactcgctggctacttttgt
MED12
gene id: 9968
asv5, Intron 21 unspliced resulting in 425 nt increase
tgatgatgctgtggtgtcattgctatgtgaatgggctgtcagctgcaagcgttctggtcggcatcgtgctatggt
ggtagccaagctcctggagaagagacaggcggagattgaggctgagcgttgtggagaatcagaagccgcagatga
gaagggttccatcgcctctggctccctttctgctcccagtgctcccattttccaggatgtcctcctgcagtttct
ggatacacaggctcccatgctgacggaccctcgaagtgagagtgagcgggtggaattctttaacttagtactgct
gttctgtgaactgattcgacatgatgttttctcccacaacatgtatacttgcactctcatctcccgaggggacct
tgcctttggagcccctggtccccggcctccctctccctttgatgatcctgccgatgacccagagcacaaggaggc
tgaaggcagcagcagcagcaagctggaagatccagggctctcagaatctatggacattgaccctagttccagtgt
tctctttgaggacatggagaagcctgatttctcattgttctcccctactatgccctgtgaggggaagggcagtcc
atcccctgagaagccagatgtcgagaaggaggtgaagcccccacccaaggagaagattgaagggacccttggggt
tctttacgaccagccacgacacgtgcagtacgccacccattttcccatcccccaggaggagtcatgcagccatga
gtgcaaccagcggttggtcgtactgtttggggtgggaaagcagcgagatgatgcccgccatgccatcaagaaaat
caccaaggatatcttgaaggttctgaaccgcaaagggacagcagaaactgaccagcttgctcctattgtgcctct
gaatcctggagacctgacattcttaggtggggaggatgggcagaagcggcgacgcaaccggcctgaagccttccc
cactgctgaagatatctttgctaagttccagcacctttcacattatgaccaacaccaggtcacggctcaggtctc
ccggaatgttctggagcagatcacgagctttgcccttggcatgtcataccacttgcctctggtgcagcatgtgca
gttcatcttcgacctcatggaatattcactcagcatcagtggcctcatcgactttgccattcagctgctgaatga
actgagtgtagttgaggctgagctgcttctcaaatcctcggatctggtgggcagctacactactagcctgtgcct
gtgcatcgtggctgtcctgcggcactatcatgcctgcctcatcctcaaccaggaccagatggcacaggtctttga
ggggctgtgtggcgtcgtgaagcatgggatgaaccggtccgatggctcctctgcagagcgctgtatccttgctta
tctctatgatctgtacacctcctgtagccatttaaagaacaaatttggggagctcttcaggtaagagaggtggaa
ggtaaggggtagcgagtgggacctactccccttcttccatgaccacccaactcaggaggagaggatggcccggga
ccctgctgcctgtctagggtcatttgtggactgtgtcctccacatactgttgtgttaccaagagtgggccctctt
cctcagcaggcttgctccccgcctatatctgtggggcccaccctcttcccccttttcctcactgccttcagaggc
cccagttccttattcccatgtggttcctttcctgcccagtctgttttgtcccatctcccttttcttgtctcaaga
tccttcatccctcactttctcctttttttcttttctcccctttcctgaccatccctcgacctcagcaggccttct
tcaacactactatctcctttcctccatccctgcagcgacttttgctcaaaggtgaagaacaccatctactgcaac
gtggagccatcggaatcaaatatgcgctgggcacctgagttcatgatcgacactctagagaaccctgcagctcac
accttcacctacacggggctaggcaagagtcttagtgagaaccctgctaaccgctacagctttgtctgcaatgcc
cttatgcacgtctgtgtggggcaccatgatcccgatagggtgaatgacatcgcaatcctgtgtgcagagctgacc
ggctattgcaagtcactgagtgcagaatggctaggagtgcttaaggccttgtgctgctcctctaacaatggcact
tgtggtttcaacgatctcctctgcaatgttgatgtcagtgacctatcttttcatgactcgctggctacttttgt
MED12
gene id: 9968
asv6, Large deletion from mid-exon 11 through exon 21, with exon 19
redefined. Also, exon 21 through exon 24 (end of clone) is intact, with no
introns
tgatgatgctgtggtgtcattgctatgtgaatgggctgtcagctgcaagcgttctggtcggcatcgtgctatggt
ggtagccaagctccacttgcctctggtgcagcatgtgcagttcatcttcgacctcatggaatattcactcagcat
cagtggcctcatcgactttgccattcaggtggggaagttggggagatgagggtggaggcaggagttcatgccata
tagcggctacggagggtcataaggacaggcgtagaggctccagccagtttcccaagcatctgctgaccctcccaa
ccttgcttcttcatgcaggctgtgtggcgtcgtgaagcatgggatgaaccggtccgatggctcctctgcagagcg
ctgtatccttgcttatctctatgatctgtacacctcctgtagccatttaaagaacaaatttggggagctcttcag
gtaagagaggtggaaggtaaggggtagcgagtgggacctactcccttcttcccatgaccacccaactcaggagga
gaggatggcccgggaccctgctgcctgtctagggtcatttgtggactgtgtcctccacatactgttgtgttacca
agagtgggccctcttcctcagcaggcttgctccccgcctatatctgtggggcccaccctcttcccccttttcctc
actgccttcagaggccccagttccttattcccatgtggttcctttcctgcccagtctgttttgtcccatctccct
tttcttgtctcaagatccttcatccctcactttctcctttttttcttttctcccctttcctgaccatccctcgac
ctcagcaggccttcttcaacactactatctcctttcctccatccctgcagcgacttttgctcaaaggtgaagaac
accatctactgcaacgtggagccatcggaatcaaatatgcgctgggcacctgagttcatgatcgacactctagag
aaccctgcagctcacaccttcacctacacggggctaggcaagagtcttagtgagaaccctgctaaccgctacagc
tttgtctgcaatgcccttatgcacgtctgtgtggggcaccatgatcccgagtatggggtgtactgagtgaggaag
ggcaccatgcccccatctgagatagggagggctgaggtacccgggaggtactacaaccttgattatttagtgggg
cagagatgagaagttaatgggtctgaggttttgtggagcaaggtttttcctgagggcatttgtacttttccctag
tagggtgaatgacatcgcaatcctgtgtgcagagctgaccggctattgcaagtcactgagtgcagaatggctagg
agtgcttaaggccttgtgctgctcctctaacaatggcacttgtggtttcaacgatctcctctgcaatgttgatgt
gagacttggggtggggttttgctagtggggcagtgaccagggcagggggctggttgtgatcctctgaccagggac
agagttccgtagagtggaggcacaccgctttgagtgggcctccacactgagtcatggtgtctgtctgttttttcc
tccaggtcagtgacctatcttttcatgactcgctggctacttttgt
MED12
gene id: 9968
asv7, Intron 24 unspliced resulting in 395 nt increase
gcagctcacaccttcacctacacggggctaggcaagagtcttagtgagaaccctgctaaccgctacagctttgtc
tgcaatgcccttatgcacgtctgtgtggggcaccatgatcccgatagggtgaatgacatcgcaatcctgtgtgca
gagctgaccggctattgcaagtcactgagtgcagaatggctaggagtgcttaaggccttgtgctgctcctctaac
aatggcacttgtggtttcaacgatctcctctgcaatgttgatgtcagtgacctatcttttcatgactcgctggct
acttttgttgccatcctcatcgctcggcagtgtttgctcctggaagatctgattcgctgtgctgccatcccttca
ctccttaatgctggtgaactaccaatctgtaacccctagcatttctagacctcaaatttcaatacacactggacg
gccatcctctcattgttcactgtgggagaccttgctgcggctccctggccttcctcagaaggccagtcctttggt
atgctgaaggctagaagaaacctgttttttagccctggatttgcagccctgacctttccaatttctgacccttca
actgcgtaacagttctctgctctacctcgctttcaatattatcttgctttttctcctttcactttacctcatctt
ctctcccatgcccctgccatacacttgcatgcatgcaggcacgcacacacataaacccacatacagtttaacttc
atcccttccagatctgttttgtcttccttttagcttgtagtgaacaggactctgagccaggggcccggcttacct
gccgcatcctccttcaccttttcaagacaccgcagctcaatccttgccagtctgatggaaacaagcctacagtag
gaatccgctcctcctgcgaccgccacctgctggctgcctcccagaaccgcatcgtggatggagccgtgtttgctg
ttctcaaggctgtgtttgtacttggggatgcggaactgaaaggttcaggcttcactgtgacaggaggaacagaag
aacttccagaggaggagggaggaggtggcagtggtggtcggaggcagggtggccgcaacatctctgtggagacag
ccagtctggatgtctatgccaagtacgtgctgcgcagcatctgccaacaggaatgggtaggagaacgttgcctta
agtctctgtgtgaggacagcaatgacctgcaagacccagtgttgagtagtgcccaggcgcagcgcctcatgcagc
tcatttgctatccacatcgactgctggacaatgaggatggggaaaacccccagcggcagcgcataaagcgcattc
tccagaacttggaccagtggaccatgcgccagtcttccttggagctgcagctcatgatcaagcagacccctaaca
atgagatgaactccctcttggagaacatcgccaaggccacaatcgaggttttccaacggtcagcagagacagggt
catc
MED12
gene id: 9968
asv8, Intron 39 unspliced resulting in 174 nt increase
cataggcctgtacacccagaaccagccactacctgcaggtggccctcgtgtggacccataccgtcctgtgcgctt
accaatgcagaagctgcccacccgaccaacttaccctggagtgctgcccacaaccatgactggcgtcatgggttt
agaaccctcctcttataagacctctgtgtaccggcagcagcaacctgcggtgccccaaggacagcgccttcgcca
acagctccaggcaaagatagtgagaggggcagtagggagggctgtcagggagaggggcttttgagggtcacagga
cggaggagacacttgggatcttcacaaggacactcagggtgggagacacaagagatgagatggcagcaagcattt
cctgagtttgagttgttctcttttctccctttagcagagtcagggcatgttgggacagtcatctgtccatcagat
gactcccagctcttcctacggtttgcagacttcccagggctatactccttatgtttctcatgtgggattgcagca
acacacaggccctgcaggtaccatggtgccccccagctactccagccagccttaccagagcacccacccttctac
caatcctactcttgtagatcctacccgccacctgcaacagcggcccagtggctatgtgcaccagcaggcccccac
ctatggacatggactgacctcc
MED12
gene id: 9968
asv9, First: Intron 39 unspliced resulting in 174 nt increase; Second: exon
41 has internal intron splice out (known ASV) which deletes 75 nts
cataggcctgtacacccagaaccagccactacctgcaggtggccctcgtgtggacccataccgtcctgtgcgctt
accaatgcagaagctgcccacccgaccaacttaccctggagtgctgcccacaaccatgactggcgtcatgggttt
agaaccctcctcttataagacctctgtgtaccggcagcagcaacctgcggtgccccaaggacagcgccttcgcca
acagctccaggcaaagatagtgagaggggcagtagggagggctgtcagggagaggggcttttgagggtcacagga
cggaggagacacttgggatcttcacaaggacactcagggtgggagacacaagagatgagatggcagcaagcattt
cctgagtttgagttgttctcttttctccctttagcagagtcagggcatgttgggacagtcatctgtccatcagat
gactcccagctcttcctacggtttgcagacttcccagggctatactccttatgtttctcatgtgggattgcagca
acacacaggccctgcagatcctacccgccacctgcaacagcggcccagtggctatgtgcaccagcaggcccccac
ctatggacatggactgacctcc
MED12
gene id: 9968
asv10, Exon 20 extended 3′, resulting in a 109 nt increase
cttgctcctattgtgcctctgaatcctggagacctgacattcttaggtggggaggatgggcagaagcggcgacgc
aaccggcctgaagccttccccactgctgaagatatctttgctaagttccagcacctttcacattatgaccaacac
caggtcacggctcaggtctcccggaatgttctggagcagatcacgagctttgcccttggcatgtcataccacttg
cctctggtgcagcatgtgcagttcatcttcgacctcatggaatattcactcagcatcagtggcctcatcgacttt
gccattcagctgctgaatgaactgagtgtagttgaggctgagctgcttctcaaatcctcggatctggtgggcagc
tacactactagcctgtgcctgtgcatcgtggctgtcctgcggcactatcatgcctgcctcatcctcaaccaggac
cagatggcacaggtctttgaggggtaagcagagcttcggaataactgaaacaaagctctggcgaatgccggtgga
agtggcctgggaagagcatgcacttcctcacactctggggaagcacctgctgctcaggctgtgtggcgtcgtgaa
gcatgggatgaaccggtccgatggctcctctgcagagcgctgtatccttgcttatctctatgatctgtacacctc
ctgtagccatttaaagaacaaatttggggagctcttcagcgacttttgctcaaaggtgaagaacaccatctactg
caacgtggagccatcggaatcaaatatgcgctgggcacctgagttcatgatcgacactctagagaaccctgcagc
tcacaccttcacctacacggggctaggcaagagtcttagtgagaaccctgctaaccgctacagctttgtctgcaa
tgcccttatgcacgtctgtgtggggcaccatgatcccgatagggtgaatgacatcgcaatcctgtgtgcagagct
gaccggctattgcaagtcactgagtgcagaatggctaggagtgcttaaggccttgtgctgctcctctaacaatgg
cacttgtggtttcaacgatctcctctgcaatgttgatgtcagtgacctatcttttcatgactcgctggctacttt
tgt
THRAP4
gene id: 9862
asv1, Extra 57 nt exon between exons 6 and 7
ccacctagaactggattgtgcgctggccgccaccgctgccacctgctcagagtgaaataatgaaggtggtcaacc
tgaagcaagccattttgcaagcctggaaggagcgctggagttactaccaatgggcaatcaacatgaagaaattct
ttcctaaaggagccacctgggatattctcaacctggcagatgcgttactagagcaggccatgattggaccatccc
ccaatcctctcatcttgtcctacctgaagtatgccattagttcccagatggtgtcctactcttctgtcctcacag
ccatcagtaagtttgatgacttttctcgggacctgtgtgtccaggcattgctggacatcatggacatgttttgtg
accgtctgagctgtcacggcaaagcagaggaatgcatcggactgtgccgagcccttcttagcgccctccactggc
tgctgcgctgcacggcagcctctgcagagcggctgcgggaggggctggaggccggcactccagccgctggggaga
agcagcttgccatgtgccttcagcgcctggagaaaaccctcagcagcaccaagaaccgggccctgctgcacatcg
ccaaactagaggaggcctcattgcacacatcccagggacttgggcagggtggcacccgagccaatcaaccaacag
cttcttggactgccatcgagcattctctcttgaaacttggagagatcctgaccaatctcagcaacccgcagctcc
ggagtcaggccgagcagtgtggcaccctcattaggagcatccccacgatgctgtctgtgcatgcggagcagatgc
acaagaccggcttccccactgtccacgccgtgatcctgctcgagggcaccatgaacctgacaggcgagacgcagt
ccctggtggagcagctgacgatggtgaagcgcatgcagcatatccccaccccactttttgtcctggagatctgga
aagcttgctt
THRAP4
gene id: 9862
asv2, First: extra exon between exons 6 and 7, (57 nt); exon 7 is extended
on the 5′ end by 315 nts
ccacctagaactggattgtgcgctggccgccaccgctgccacctgctcagagtgaaataatgaaggtggtcaacc
tgaagcaagccattttgcaagcctggaaggagcgctggagttactaccaatgggcaatcaacatgaagaaattct
ttcctaaaggagccacctgggatattctcaacctggcagatgcgttactagagcaggccatgattggaccatccc
ccaatcctctcatcttgtcctacctgaagtatgccattagttcccagatggtgtcctactcttctgtcctcacag
ccatcagtaagtttgatgacttttctcgggacctgtgtgtccaggcattgctggacatcatggacatgttttgtg
accgtctgagctgtcacggcaaagcagaggaatgcatcggactgtgccgagcccttcttagcgccctccactggc
tgctgcgctgcacggcagcctctgcagagcggctgcgggaggggctggaggccggcactccagccgctggggaga
agcagcttgccatgtgccttcagcgcctggagaaaaccctcagcagcaccaagaaccgggccctgctgcacatcg
ccaaactagaggaggcctcattgcacacatcccagggacttgggcagggtggcacccgagccaatcaaccaacag
ccactggattctggcctccctctgcctctctctcctgagcctgtgtgatgccataccttctgaagtcagctggct
gtgtcccctggaaatcaggcttttgggaatggtctctggggtttccagctctaggtgcccaccccccttctggaa
acagtgcatgctgccctcaggcccctccctccctgttgtcctcaggggaagccttcctgtgtggtttcgtgtgcc
ggagggagtgccaaaatcgaggagttcagggccaggtgctccttctctcctgtttcccatcatgtttctgtactt
ccttccctctgccagcttcttggactgccatcgagcattctctcttgaaacttggagagatcctgaccaatctca
gcaacccgcagctccggagtcaggccgagcagtgtggcaccctcattaggagcatccccacgatgctgtctgtgc
atgcggagcagatgcacaagaccggcttccccactgtccacgccgtgatcctgctcgagggcaccatgaacctga
caggcgagacgcagtccctggtggagcagctgacgatggtgaagcgcatgcagcatatccccaccccactttttg
tcctggagatctggaaagcttgctt
THRAP3
gene id: 9967
asv1, Extra exon (192 nt), located 114 nt after exon 8
ggaacaggagtttcgttccattttccagcacatacaatcagctcagtctcagcgtagcccctcagaactgtttgc
ccaacatatagtgaccattgttcaccatgttaaagagcatcactttgggtcctcaggaatgacattacatgaacg
ctttactaaatacctaaagagaggaactgagcaggaggcagccaaaaacaagaaaagcccagagatacacaggag
aatagacatttcccccagtacattcagaaaacatggtttggctcatgatgaaatgaaaagtccccgggaacctgg
ctacaaggatgggcataattctaaaaatgaactacaaagggttaatttttattaaatgtatcaacaacctttgtg
aagtggttagaatatggtaaatgaccccaaagtctattgaggtgagcttgagaaaaaaaagagaggagttttgga
acaagtgcccatgatgagagaagaaactttttgtgatatttttctgcttgctgagggaaaatacaaagatgatcc
tgttgatctccgccttgatattg
HMG20B
gene id: 10362
asv1, Exon 5 spliced out, loss of 216 nt
acggagaagatccaggagaagaagatcaagaaagaagactcgagctctgggctcatgaacactctcctgaatgga
cacaagggtggggactgcgatggcttctccaccttcgatgttcccatcttcactgaagagttcttggaccaaaac
aaaggcacgggcgaaacgcccacgctgggcactctggacttctacatggcccggcttcacggagccatcgagcgc
gaccccgcccagcacgagaagctcatcgtccgcatcaaggaaatcctggcccaggtcgccagcgagcacctgtga
ggagtgggcgggcccacgatgcagaggagaagctgtgggcgcggccctgccacaccccaccccgtggacgagagg
ctgggggtccaccctttggggcctggtcccatcctgcacctttgggggctccagcccccctaaaattaaatttct
gcagcatccctttagctttcaatctccccagccccctgaacccggaaaaagcactcgctgcgcgatacacccaga
agaacctcacagccgagggtgcccctcctcggaggacagccacgcgctacactggctctccgggccacccccagg
acacagggcagacgaaacccacccccagcacacggcaggaccccccaaattactcactacggggggctgtgccat
aggccacacaggaagctgccttgtggggacttacctggggtgtcccccgcatgcctgtaccccagatgggtgggg
gccggctttgcccatcctgctctcctccagccgagggaccctggtgggggtggctccttctcactgctggatcc
OGHDL
gene id: 55753
asv1, exon 10 extended 5′
caggggaaggctgaacgtgctggccaacgtgatccgcaaggacctggagcagatcttctgccagtttgaccccaa
gctggaggcggcggacgagggctccggggatgtcaagtaccacctgggcatgtaccacgagaggatcaaccgcgt
caccaaccggaacatcactctgtcgctggttgccaacccctcccacctggaggcagtggaccctgtggtgcaggg
gaagacaaaggcagagcagttctaccgtggagatgcccagggcaagaagcccctcctggctcacacctgccctgc
aggtcatgtccatcctggttcatggggacgccgcctttgctggccagggcgtggtatatgagaccttccacctga
gcgacctgccctcctacacgaccaatggtaccgtgcacgtcgtcgtcaacaaccagattggattcaccacagacc
cccgaatggcccgctcctcaccatacccgaccgacgtggcccgggtggtcaatgcgcctatcttccatgtgaatg
ccgatgacccaaaggctgtgatatatgtgtgcagtgtggca
HRNP
wildtype = NM_031243
exon 2 deleted; deletion of 36 nucleotides
HRNP asv1
GACGAGTCCGGTTCGTGTTCGTCCGCGGAGATCTCTCTCATCTCGCTCGGCTGCGGGAAATCGGGCTGAAGCGAC
TGAGTCCGCGATGGAGAGAGAAAAGGAACAGTTCCGTAAGCTCTTTATTGGTGGCTTAAGCTTTGAAACCACAGA
AGAAAGTTTGAGGAACTACTACGAACAATGGGGAAAGCTTACAGACTGTGTGGTAATGAG
BACS1
wildtype = AF041260
exons 9 and 10 deleted; deletion of 234 nucleotides
BACS1/1 asv1
GCGAAGGAAGGCACCAAGGAGAAATCAGGACCCACCTCTCTGCCTCTGGGCAAACTGTTTTGGAAAAAGTCAGTT
AAAGAGGACTCAGTCCCCACAGGTGCGGAGGAGAATACATCAGACTCCACAGAAAAGACTATCACACCGCCAGAG
CCTGAACCAACAGGAGCACCACAGAAGGGTAAAGAGGGCTCCTCGAAGGACAAGAAGTCA
ATF4
wildtype = D90209
Intron retention between exons I and II, splicing occurs in 5′UTR.
atf4 asv1
GCAGCAGCACCAGGCTCTGCAGCGGCAACCCCCAGCGGCTTAAGCCATGGCGTGAGTACCGGGGCGGGTCGTCCA
GCTGTGCTCCTGGGGCCGGCGCGGGTTTTGGATTGGTGGGGTGCGGCCTGGGGCCAGGGCGGTGCCGCCAAGGGG
GAAGCGATTTAACGAGCGCCCGGGACGCGTGGTCTTTGCTTGGGTGTCCCCGAGACGCTCGCGTGCCTGGGATCG
GGAAAGCGTAGTCGGGTGCCCGGACTGCTTCCCCAGGAGCCCTACAGCCCTCGGACCCCGAGCCCCGCAAGGTCC
CAGGGGTCTTGGCTGTTGCCCCACGAAACGTGCAGGAACCAAGATGGCGGCGGCAGGGCGGCGGCGCGGGCGTGA
GTCAAGGGCGGGCGGTGGGCGGGGCGCGGCCGCTGGCCGTATTTGGACGTGGGGACGGAGCGCTTTCCTCTTGGC
GGCCGGTGGAAGAATCCCCTGGTCTCCGTGAGCGTCCATTTTGTGGAACCTGAGTTGCAAGCAGGGAGGGGCAAA
TACAACTGCCCTGTTCCCGATTCTCTAGATGGCCGATCTAGAGAAGTCCCGCCTCATAAGTGGAAGGATGAAATT
CTCAGAACAGCTAACCTCTAATGGGAGTTGGCTTCTGATTCTCATTCAGGCTTCTCACGGCATTCAGCAGCAGCG
TTGCTGTAACCGACAAAGACACCTTCGAATTAAGCACATTCCTCGATTCCAGCAAAGCACCGCAACATGACCGAA
ATGAGCTTCCTGAGCAGCGA
BTF3
wildtype = X53280
Alternative exon 1, N-terminally truncated protein, sequence identical to
constitutive variant.
btf3 asv1
GCCATCTTGCGTCCCCGCGTGTGTGCGCCTAATCTCAGGTGGTCCACCCGAGACCCCTTGAGCACCAACCCTAGT
CCCCCGCGCGGCCCCTTATTCGCTCCGACAAGATGAAAGAAACAATCATGAACCAGGAAAAACTC
CENPA
wildtype = CD628726
Exon 2 skipping; deletion of 73 nucleotides
cenpa asv1
GGTCCGCCGACATGGCCTGGACCAAGTACCAGCTGTTCCTGGCCGGGCTCATGCTTGTTACCGGCTCCATCAACA
CGCTCTCGGCAAAGCAGTGGGCATGTTCCTGGGAGAATTCTCCTGCCTGGCTGCCTTCTACCTCCTCCGATGCAG
AGCTGCAGGGCAATCAGACTCCAGCGTAGAC
Msx2
wildtype = D89377
Deletion in exon 2; deletion of 1317 nucleotides
C-terminal truncated protein is produced, sequence is identical to
constitutive variant.
msx2 asv1
CCTGGAGCGCAAGTTCCGTCAGAAACAGTACCTCTCCATTGCAGAGCGTGCAGAGTTCTCCAGCTCTCTGAACCT
CACAGAGACCCAGGTCAAAATCTGGTTCCAGAACAGAAGGTAAAGCCATGTTTTGACTTGGTGAAAATGGGGTTG
TCAAACAGCCCATTAAGCTCCCTGGTATTT
NFIC
wildtype = BC012120
Deletion in exon 7, exon 8 deleted, alternative exon after exon 7
nfic asv1
GGCATCTCGTCCCCGGTGAAGAAGACAGAGATGGACAAGTCACCATTCAACAGCACGTCCCCTGCAAACCGTTCC
TTTGTGGGATTAGGACCAAGGGATCCTGCGGGCATTTATCAGGCACAGTCCTGGTATCTGGGATAGCAAAGGTCT
TCTTCCCTCGCCCCTTCTCCATCGTCCCAGGAATCCCAGGGGGCAGCACAGCCGGCCCCCGGCCCACGTTTTCGG
TGGAAAATTAGAGTG
RELA
wildtype = L19067
deletion of 341 nucleotides
rela/1 asv1
CGTGCCCCCAACACTGCCGAGCTCAAGATCTGCCGAGTGAACCGAAACTCTGGCAGCTGCCTCGGTGGGGATGAG
ATCTTCCTACTGTGTGACAAGGTGCAGAAAGAGGACATTGAGGTGTGTCCCCAAGCCAGCACCCCAGCCCTATCC
CTTTACGTCATCCCTGAGCACCATCAACTATGATGAGTTTCCCACCATGGTGTTTCCTTC
SNAI1
wildtype = BC012910
Different 5′ exon, deletions in exons 2 and 3; deletion of 1085 nucleotides
snai1 asv1
ACAGCGAGCTGCAGGACTCTAATCCAGAGTTTACCTTCCAGCAGCCCTACGACCAGGCCCACCTGCTGGCAGCCA
TCCCACGAGGTGTGACTAACTATGCAATAATCCACCCCCAGGTGCAGCCCCAGGGCCTGCGGAGGCGGTGGCAGA
CTAGAGTCTGAGATGCCCCGAGCCCAGGCA
TFE3
wildtype = X96717
Deletion in exons 8 and 10, exon 9 deleted; deletion of 1032 nucleotides
TFE3 asv1
TGTCAGCAACTCCTGCCCAGCTGAGCTGCCCAACATCAAACGGGAGATCTCTGAGACCGAGGCAAAGGCCCTTTT
GAAGGAACGGCAGAAGAAAGACAATCACAACCTAATTGAGCGTCGCAGGCGATTCAACATTAACGACAGGATGTT
GCTCCATCCTTTGTCTTGGAACCACCAGTCTAGTCCGTCCTGGCACAGAAGAGGAGTCAAGTAATGGAGGTCCCA
GCCCTGGGGGTTTAAGCTCTGCCCCTTCCCCATGAACCCTGCCCTGCTCTGCCCA
CD44
wildtype = BC004372
Exons 6-11 deleted; deletion of 618 nucleotides
cd44/1 asv1
TTACACCTTTTCTACTGTACACCCCATCCCAGACGAAGACAGTCCCTGGATCACCGACAGCACAGACAGAATCCC
TGCTACCAATATGGACTCCAGTCATAGTACAACGCTTCAGCCTACTGCAAATCCAAACACAGGTTTGGTGGAAGA
TTTGGACAGGACAGGACCTCTTTCAATGACAACGCAGCAGAGTAATTCTCAGAGCTTCTC
NEMP
wildtype = Y11392
Exon 6 cryptic splicing; insertion of 360 nucleotides
nemp asv1
AGCCGCCTTCCCGGGGCCAGTTTCCTTCCCTCTCAGCCAGGGATGCCTCGAGCAGCCACAGGGGCAGGGTGAGTG
GCGGGCCGCTAGGGGCCGCGGCTGCCTCTGCCCACTGCACCCACTGCACAGAAACCGTGGGGAGGGAGCATGGAG
CCTCACAGGGCCCCGTGGGGAGGGAGCATGGAGCCTCACAGGGCCTTGAAGAGCTGTGCCCCAGGGGGAGCTGCG
TGTGCGGGTCTGTGAATGCGCACACACGTGTAACACGTGCCCCGCACGGAGCCGTCCTGGCCCCTCAGCCTCTCC
TGCTGTCCTGGTCTGTGGAATGTGGGCCCGGGCCCTGCTGGGCTGAGGGCAACAGGAGTCACGTGGAAGAGGTGC
CACACACGCGTCCACAGGCGGGGCTCCTCTGCTCAGATTCTCCGAGTGTGCCGAACGTCCTGACTGCCATCCTGC
TGCTGCTGCGGGAGCTGGATGCAGAGGGGCTGGAGGCCGT
HDAC5
wildtype = AB011172
Exons 14 and 15 in; insertion of 255 nucleotides
hdac5 asv1
TGCTGCCCCTGGGGGCATGAAGAGCCCCCCAGACCAGCCCGTCAAGCACCTCTTCACCACAGGTGTGGTCTACGA
CACGTTCATGCTAAAGCACCAGTGCATGTGCGGGAACACACACGTGCACCCTGAGCATGCTGGCCGGATCCAGAG
CATCTGGTCCCGGCTGCAGGAGACAGGCCTGCTTAGCAAGTGCGAGCGGATCCGAGGTCGCAAAGCCACGCTAGA
TGAGATCCAGACAGTGCACTCTGAATACCACACCCTGCTCTATGGGACCAGTCCCCTCAACCGGCAGAAGCTAGA
CAGCAAGAAGTTGCTCGGCCCCATCAGCCAGAAGATGTATGCTGTGCTGCCTTGTGGGGGCATCGGGGTGGACAG
TGACACCGTGTGGAATGAGATGCACTCCTCCAGTGCTGTGCGCAT
EST
wildtype = AL037524
Additional exon spliced in; insertion of 120 nucleotides
est asv1
GTTTAGTGTCTTTTCCTTGTNTCTGCTCGGGGAGCGTGAGGCAGATCGGCCGGCTTTGCTCCAGGCCTCAGGAGT
GTCACTCGCCTNGGCTTGCACAGTACATTGGAACGTGCGGGTTCTATTTTGTATTCGACGTGCCGGATCGAAATA
GAGCTCGCGGCACTNTGAAGACCACAGTAGGAAGTTAAGGACGGGGGTGCAGGTTCGCAGCCCTATCAACCAGCT
CCGAGCC
SUA1
wildtype = AK021978
Additional exon spliced in after exon 3; insertion of 58 nucleotides
sua1 asv1
GATGTGAAGGTGGACACTGAGGATATGGAGAAGAAACCAGAGTCATTTTTCACTCAATTCGATGCTATGGGATTT
TTCCTTGGGTGGCTGCATTCTTTGAAACACCAAAGGAACACATTTCTCTGTGTGTCTGACTTGCTGCTCCAGGGA
TGTCATAGTTAAAGTTGACCAGATCTGTCA
POMT1
wildtype = BC022877
Extended exon 8.; insertion of 66 nucleotides
pomt1 asv1
TCCTGTGCAGTGGGCATCAAGTACATGGGTGTGTTCACGTACGTGCTCGTGCTGGGTGTTGCAGCTGTCCATGCC
TGGCACCTGCTTGGAGACCAGACTTTGTCCAATGTAGGTGCTGATGTCCAGTGCTGCATGAGGCCGGCCTGTATG
GGGCAGATGCGGATGTCACAGGGGGTCTGTGTGTTCTGTCACTTGCTCGCCCGAGCAGTGGCTTTGCTGGTCATC
CCGGTCGTCCTGTACTTACTGTTCTTCTACGTCCACTTGATTCTAGTCTTCCGCT
TGIF
wildtype = NM_170695
Alternative splice donor in exon 1; deletion of 607 nucleotides, protein is
truncated at the N-terminus, but identical to constitutive form.
tgif asv1
GGCTGCGTTTCTGTGGGAGGCCCTGAAACGCGCGGAGCTTCCCTCTGCCTCCAGGCTTTCCCAGCGAGAGTGAAA
TTAAACTTGAAACTCGGATCAACTGGCAGTCGTTGTTGGTATTGTTGCAGCATCTGGCAGTGAGACTGAGGATGA
GGACAGCATGGACATTCCCTTGGACCTTTCTTCATCCGCTGGCTCAGGCAAGAGAAGGAG
galectin 9
wildtype = AB006782
Exon 6 spliced out; deletion of 36 nucleotides
galectin 9 asv1
CCTGTTCAGCCTGCCTTCTCCACGGTGCCGTTCTCCCAGCCTGTCTGTTTCCCACCCAGGCCCAGGGGGCGCAGA
CAAAAAACCCAGACAGTCATCCACACAGTGCAGAGCGCCCCTGGACAGATGTTCTCTACTCCCGCCATCCCACCT
ATGATGTACCCCCACCCCGCCTATCCGATG
Oct11a
wildtype = AF133895
Exon 10 spliced out; deletion of 162 nucleotides
oct11a asv1
TGGTAGGAAGAGAAAGAAACGGACCAGCATCGAGACCAACATCCGCCTGACTCTGGAGAAGAGGTTTCAAGATGT
ATCTCCCTCAGGGTCTCTGGGCCCCCTCTCTGTCCCTCCTGTCCACAGTACCATGCCTGGAACAGTAACGTCATC
CTGTTCCCCTGGGAACAACAGCAGGCCTTC
CA11
wildtype = AF067662
Exons 2-6 and the first half of exon 7 spliced out; deletion of 621
nucleotides
ca11 asv1
GGGGATGGGGGCTGCAGCTCGTCTGAGCGCCCCTCGAGCGCTGGTACTCTGGGCTGCACTGGGGGCAGCAGCTCA
CATCGGACCATCACCTATCAGGGCTCTCTCAGCACCCCGCCCTGCTCCGAGACTGTCACCTGGATCCTCATTGAC
CGGGCCCTCAATATCACCTCCCTTCAGATG
GPX2
wildtype = X53463
Additional exon after exon 1; insertion of 200 nucleotides
gpx2 asv1
ACCCGGGACTTCACCCAGCTCAACGAGCTGCAATGCCGCTTTCCCAGGCGCCTGGTGGTCCTTGGCTTCCCTTGC
AACCAATTTGGACATCAGGAGAGACAGAAGTAGCAAACCCTCTTTCGAGATGTCCCTCCAGCCCCAGAAGTACCT
CCAGCCTCACACCATCTCTTCAGCCTAGCAAGTTGCTGGAGGGAGTCTATAACCTACCAGGAGCCAGCCAGCCAT
TGTATCAAGAAATAGAAATCTGCCAGGTACAGGGCTCACACCTATAATCCCAGCGCTTGGGAGGCTAAGGAGAAC
AGTCAGAATGAGGAGATCCTGAACAGTCTCAAGTATGTCCGTCCTGGGGGTGGATACCAG
MAX
wildtype = BC036092
Alternative 3′exon after exon 3
max asv1
CCACATCAAAGACAGCTTTCACAGTTTGCGGGACTCAGTCCCATCACTCCAAGGAGAGAAGCTCTATTTCCTCTT
TTGGAAATTGTGTACTCCTGTCCTTCATCGTCAAAGTTTGATGCAGAAATGCCACACCTTCATTTCAAGCTACCA
AGTGCACAAGAAAAAAGAATGCAAGATTTAAAAAATGATTGTTTTGACCCCTTACACAAATGTCTTACTCCTGGC
TTTAATTAAGCTGCTTGAGGGCTGATAGCTCTGCCTTACCCTGGTAATCAGCAAAATGGTCCTGTGGCTGGGGAG
GCCCTGGCAGCAGGAAGCCTTCAAGGAGCCATGGGTCTGTGCTGACTCTGGCCTTACAACCTTCCAGCCTCCTTT
GCTGGCATTGATGGGGTTCCATTTTTGAATGAACTAGTTTAATGTGGATCCAAATTTATTGTGCATATTCTTTCG
TTTTGGTTTTCAAAAGATGGCTTATTCACATGGAAATGTACACCAGTTTAGCCCTGGGCCCTCCCTTTACCTTCA
TATGTGTAAAAGCTTACACAGGTTTCAGAAAATAAATGGTTTCATTTTCTCTAAAATAACTAGTACAAAATAAAA
CAGATGTCAGTTGTTGAAAAAAAAAAAAAAAAAAAAAAAAAAAAAAAAAAAAAAAAAAAAAA
PPARG
wildtype = NM_138712
Alternative 5′ exon, does not change the protein
pparg asv1
CCAGAAGCCTGCATTTCTGCATTCTGCTTAATTCCCTTTCCTTAGATTTGAAAGAAGCCAACACTAAACCACAAA
TATACAACAAGGCCATTTTCTCAAACGAGAGTCAGCCTTTAACGAAATGACCATGGTTGACACAG
CCRG
wildtype = NM_032579
Alternative 3′ exon, protein composition is not changed.
ccrg asv1
GTGACCATGACAGTAATGAAACCAGGGTCCCAACCAAGAAATCTAACTCAAACGTCCACTTCATTTGTTCCATTC
CTGATTCTTGGGTAATAAAGACAAACTTTGTACCTCTCAAAAAAAAAAAAAAAAAAGTTGGCCTGCAGGCGGCCG
CAGGTAAGCCAGCCCAGGCCTCGCCCTCCAGCTCAAGGCGGGACAGGGC
SDCCAG1
wildtype = NM_004713
One exon skipped and one exon inserted
SDCCAG1 asv1
GCAATCAAAGAATTAAAACTACAAACAAACCATGTTACAATGCTGCTAAGAGGAGGAAGATGATGATGTTGATGG
TGACGTCAATGTTGAGAAAAATGAAACTGAACCACCAAAAGGAAAAAAGAAAAAACAAAAGAATAAACAGCTGCA
GAAGCCTCAGAAAATAAGCCCCTTACTTGTAGATGTTGATCTCAGCTTGTCAGCATATGCCAATGCCAAAAAGTA
TTATGATCACAAGAGATATGCTGCTAAGAAAACACAAAAGACTGTTGAAGCTGCTGAGAAGGCATTCAAGTCAGC
AGAAAAGAAAACAAAGCAAACATTAAAAGAAGTTCAGACTGTTACCTCTATTCAAAAAGCAAGAAAAGTATATTG
CTTAGGATTCAGCTTCTTAAGTCTGATCACAGCCGGGCGCAGTGGCTCACGCCTGTAATCCCAGCACTTTGGGAG
GCCGAGGAGGGCGGATCACGAGGTCAAGAGATCGAGACCATCCTGGCTAACACGGTGGACGAGATCAGCAACAGA
ATGAAATAATTGTGAAAAGATACTTGACACCAGGAGACATTTATGTACATGCTGATCTTCATGGAGCTACTAGCT
GTGTAATTAAGAATCCAACAGGAGAACCCATCCCCCCACGGACCTTGACTGAAGCTGGCACAATGGCACTTTGCT
ACAGTGCTGCTTGGGATGCACGAGTTATCACTAGTGCTTGGTGGGTGTACCATCATCAGGTATCTAAAACAGCAC
CAACTGGAGAATATTTGACAACAGGAAGCTTCATGATAAGAGGAAAAAAGAATTTTCTTCCTCCCTCATATCTAA
TGATGGGGTTTAGCTTCCTTTTTAAGGTAGATGAGTCTTGTGTTTGGAGACATCAGGGTGAACGAAAAGTCAGAG
TACAGGATGAAGACATGGAGACACTGGCAAGTTGTACAAGTGAACTCATATCAGAAGAAATGGAACAATTAGATG
GAGGTGACACGAGCAGTGATGAGGATAAAGAAGAACATGAAACTCCTGTGGAAGTAGAACTCATGACTCAGGTTG
ACCAAGAGGATATCACTCTTCAGAGTGGCAGAGATGAACTAAATGAGGAGCTCATTCAGGAAGAAAGCTCTGAAG
ACGAAGGAGAATATGAAGAGGTTAGAAAAGATCAGGATTCTGTTGGTGAAATGAAGGATGAAGGGGAAGAGACAT
TAAATTATCCTGATACTACCATTGACTTGTCTCACCTTCAACCCCAAAGGTCCATCCAGAAATTGGCTTCAAAAG
AGGAATCTTCTAATTCTAGTGACAGTAAATCACAGAGCCGGAGACATTTGTCAGCCAAGGAAAGAAGTAGAGATG
GGGTTTCACCGTGTTGGGCAGGATTGTCTCGATCTTCTGACCTCGCGATCCACCCGCCTTGGCCTCCCAAAGTGC
TGGATTACAGTCAACCAACCGGTCAACAGATGTTTTATTGAATGCCTAAGACCTGCCAATGCTATGTTGGTACAA
AGACTACAAATCCCAGTGCCTGGCCATCAAGGGAAATGAAAAAGAAAAAACTTCCAAGTGACTCAGGAGATTTAG
AAGCGTTAGAGGGAAAGGATAAAGAAAAAGAAAGTACTGTACACA
SDCCAG10
wildtype = BC012117
Intron retention in 5′UTR
SDCCAG10 asv1
GCTGGAGATATTGACATAGAGTTGTGGTCCAAAGAAGCTCCTAAAGCTTGCAGAAATTTTATCCAACTTTGTTTG
GAAGCTTATTATGACAATACCATTTTTCATAGAGTTGTGCCTGGTTTCATAGTCCAAGGCGGAGATCCTACTGGC
ACAGGGAGTGGTGGAGAGTCTATCTATGG
SDCCAG8
wildtype = AF039690
Exon 3 insertion; insertion of 192 bp
SDCCAG8 asv1
CAGGAGCTGACACAGAAGATACAGCAAATGGAGGCCCAGCATGACAAAACTGAAAATGAACAGTATTTGTTGCTG
ACCTCCCAGAATACATTTTTGACAAAGTTAAAGGAAGAATGCTGTACATTAGCCAAGAAACTGGAACAAATCTCT
CAAAAAACCAGATCTGAAATAGCTCAACTCAGTCAAGAAAAAAGGTATACATATGATAAATTGGGAAAGTTACAG
AGAAGAAATGAAGAATTGGAGGAACAGTGTGTCCAGCATGGGAGAGTACATGAGACGATGAAGCAAAGGCTAAGG
CAGCTGGATAAGCACAGCCAGGCCACAGCCCAGCAGCTGGTGCAGCTCCTCAGCAAGCAG
NY-BR-20
wildtype = AF308287
Exon 2 skipping, exon 3 insertion. Alternative ATG.
NY-BR-20 asv1
GGCTGGAGGAAAGGGAACTGAACGCGGTTCTGGGAGCAGCAAGCCCACGGGTAGCAGCCGAGGCCCCAGAATGAG
TACAAGGAATGCTTCTCCCTGTATGACAAGCAGCAGAGGGGGAAGATAAAAGCCACCGACCTCATGGTGGCCATG
AGGTGCCTGGGGGCCAGCCCGACGCCAGGGGAGGTGCAGCGGCACCTGCAGACCCACGGGATAGACGGAAATGGA
GAGCTGGATTTCTCCACTTTTCTGACCATTATGCACATGCAAATAAAACAAGAAGACCCAAAGAAAGAAATTCTT
EPSTI1
wildtype = NM_033255
Two additional exons spliced in.
EPSTI1 asv1
CAGAATCGCCAGACAGAAGTGCCTGTCAAAGTGCTGTTTGTGGCCCACAATCCTCAACATGGAAACTTCCTATCC
TGCCTAGGGATCACAGCTGGGCCAGAAGCTGGGCTTACAGAGATTCTCTAAAGGCAGAAGAAAACAGAAAATTGC
AAAAGATGAAGGATGAACAACATCAAAAGAGTGAATTACTGGAACTGAAACGGCAGCAGCAAGAGCAAGAAAGAG
CCAAAATCCACCAGACTGAACACAGGAGGGTAAATAATGCTTTTCTGGACCGACTCCAAGGCAAAAGTCAACCAG
GTGGCCTCGAGCAATCTGGAGGCTGTTGGAATATGAATAGCGGTAACAGCTGGGGTTCTCTATTAGTTTTTTCGA
GGCACCTAAGGGTATATGAGAAAATATTGACTCCTATCTGGCCTTCATCAACTGACCTCGAAAAGCCTCATGAGA
TGCTTTTTCTTAATGTGATTTTGTTCAGCC
PPP1R1B
wildtype = AF435975
Cryptic splicing in exon I (results in extended ORF), exons III and IV
spliced out
PPP1R1B asv1
AGAGACACACGCGGAGAGGAGGAGAGGCTGAGGGAGGGAGGTGGAGAAGGACGGGAGAGGCAGAGAGAGGAGACA
CGCAGAGACACTCAGGAGGGGAGAGACACCGAGACGCAGAGACACTCAGGAGGGGAGAGACACCGAGACGCAGAG
ACACCCAGGCCGGGGAGCGCGAGGGAGCGAGGCACAGACCTGGCCCAGCCCGGGCGCCGACCCTCCTCCCGCTCC
CGCGCCCTCCCCTCGGCGGGCACGGTATTTTTATCCGTGCGCGAACAGCCCTCCTCCTCCTCTCGCCGCACAGCC
ACCAACGCCTGCCATGCTGTTCCGGCTCTCAGAGCACTCCTCACCAGCTGTGCAGCGCATTGCTGAGTCTCACCT
GCAGTCTATCAGCAATTTGAATGAGAACCAGGCCTCAGAGGAGGA
USH1C
wildtype = AF250731
Exon 11 skipping
USH1C asv1
GTGGGATTGGAGATAGGGGACCAGATTGTCGAAGTCAATGGCGTCGACTTCTCTAACCTGGATCACAAGGAGGGC
CGGGAGCTGTTCATGACAGACCGGGAGCGGCTGGCAGAGGCGCGGCAGCGTGAGCTGCAGCGGCAGGAGCTTCTC
ATGCAGAAGCGGCTGGCGATGGAGTCCAAC
USH1C
wildtype = AF250731
Exon 7 skipping
USH1C asv2
CTGATCCCCGTGAAAAGCTCTCCTGATGAGCCCCTCACTTGGCAGTATGTGGATCAGTTTGTGTCGGAATCTGGG
GGCGTGCGAGGCAGCCTGGGCTCCCCTGGAAATCGGGAAAACAAGGAGAAGAAGGTCTTCATCAGCCTGGTAGGC
TCCCGAGGCCTTGGCTGCAGCATTTCCAGC
BRD3
wildtype = D26362
Alternative 5′ and 3′ exons.
brd3 asv1
GTTTACAAACACGGGCTCCCGGCAGGTGCGCGCCGCCCCGCCCGTGCGCGGCCGGGGTTCGAGGGTGGCTCCCGC
GGGCCTCGGGGTGCCCGGACGGGGGCTGCGGTGCTGGCTGCGTGCCCGCTTCTTCCATGCCGTCCTGGGGCACCG
GAAAATCCGCCGCCAGGCGCTGTCCCCGACACGGGCTGTCGCCTGGTTGGGCCCGGAAATGGGACGTCGCGCTTT
CTCAGGGAGCGTAGAAGCAGCCAGGGCCTCTCCAAGCCGCTGCTGTGACAGAAAGTGAGTGAGCTGCCGGAGGAT
GTCCACCGCCACGACAGTCGCCCCCGCGGGGATCCCGGCGACCCCGGGCCCTGTGAACCCACCCCCCCCGGAGGT
CTCCAACCCCAGCAAGCCCGGCCGCAAGACCAACCAGCTGCAGTACATGCAGAATGTGGTGGTGAAGACGCTCTG
GAAACACCAGTTCGCCTGGCCCTTCTACCAGCCCGTGGACGCAATCAAATTGAACCTGCCGGATTATCATAAAAT
AATTAAAAACCCAATGGATATGGGGACTATTAAGAAGAGACTAGAAAATAATTATTATTGGAGTGCAAGCGAATG
TATGCAGGACTTCAACACCATGTTTACAAATTGTTACATTTATAACAAGCCCACAGATGACATAGTGCTAATGGC
CCAAGCTTTAGAGAAAATTTTTCTACAAAAAGTGGCCCAGATGCCCCAAGAGGAAGTTGAATTATTACCCCCTGC
TCCAAAGGGCAAAGGTCGGAAGCCGGCTGCGGGAGCCCAGAGCGCAGGTACACAGCAAGTGGCGGCCGTGTCCTC
TGTCTCCCCAGCGACCCCCTTTCAGAGCGTGCCCCCCACCGTCTCCCAGACGCCCGTCATCGCTGCCACCCCTGT
ACCAACCATCACTGCAAACGTCACGTCGGTCCCAGTCCCCCCAGCTGCCGCCCCACCTCCTCCTGCCACACCCAT
CGTCCCCGTGGTCCCTCCTACGCCGCCTGTCGTCAAGAAAAAGGGCGTGAAGCGGAAAGCAGACACAACCACTCC
CACGACGTCGGCCATCACTGCCAGCCGGAGTGAGTCGCCCCCGCCGTTGTCAGACCCCAAGCAGGCCAAAGTGGT
GGCCCGGCGGGAGAGTGGTGGCCGCCCCATCAAGCCTCCCAAGAAGGACCTGGAGGACGGCGAGGTGCCCCAGCA
CGCAGGCAAGAAGGGCAAGCTGTCGGAGCACCTGCGCTACTGCGACAGCATCCTCAGGGAGATGCTATCCAAGAA
GCACGCGGCCTACGCCTGGCCCTTCTACAAGCCAGTGGATGCCGAGGCCCTGGAGCTGCACGACTACCACGACAT
CATCAAGCACCCGATGGACCTCAGCACCGTGAAAAGGAAGATGGATGGCCGAGAGTACCCAGACGCACAGGGCTT
TGCTGCTGATGTCCGGCTGATGTTCTCGAATTGCTACAAATACAATCCCCCAGACCACGAGGTTGTGGCCATGGC
CCGGAAGCTCCAGGACGTGTTTGAGATGAGGTTTGCCAAGATGCCAGATGAGCCCGTGGAGGCACCGGCGCTGCC
TGCCCCCGCGGCCCCCATGGTGAGCAAGGGCGCTGAGAGCAGCCGTAGCAGTGAGGAGAGCTCTTCGGACTCAGG
CAGCTCGGACTCGGAGGAGGAGCGGGCCACCAGGCTGGCGGAGCTGCAGGAGCAGCTGAAGGCCGTGCACGAGCA
GCTGGCCGCCCTGTCTCAGGCCCCAGTAAACAAACCAAAGAAGAAGAAGGAGAAGAAGGAGAAGGAGAAGAAGAA
GAAGGACAAGGAGAAGGAGAAGGAGAAGCACAAAGTGAAGGCCGAGGAAGAGAAGAAGGCCAAGGTGGCTCCGCC
TGCCAAGCAGGCTCAGCAGAAGAAGGCTCCTGCCAAGAAGGCCAACAGCACGACCACGGCCGGCAGAGATCATTT
CTTGACCTGTGGAGTTTGAGACGCCTATGGGGTGTAGAGAGGAACGAACCTCTGTAATTGTTTCCTGGCCAAGGG
CTGGAAACCCCGCAGCTGGGAGCGACTTTTCTAACCTTGGATTTTCTGCCTTGGGGCACCACTTTGGGAAGAAAG
CTTGGTCCCAGAGAGCAGCCTGCTGTTGGGAGGAAGGGGTGTGTGCAGTGGGCTCCCACGGCAGGTAGACGGAGA
CTCAACACCACGTTGCTCTGTCTCCTGCCCCAGACAGCTGAAGAAAGGCGGCAAGCAGGCATCTGCCTCCTACGA
CTCAGAGGAAGAGGAGGAGGGCCTGCCCATGAGCTACGATGAAAAGCGCCAGCTTAGCCTGGACATCAACCGGCT
GCCCGGGGAGAAGCTGGGCCGGGTAGTGCACATCATCCAATCTCGGGAGCCCTCGCTCAGGGACTCCAACCCCGA
CGAGATAGAAATTGACTTTGAGACTCTGAAACCCCCCCCTTTGCGGGAACTGGAGAGATATGTCAAGTCTTGTTT
ACAGAAAAAGCAAAGGAAACCGTTCTGTAAAAAAAAAAAAAAAAAAAAAAAAAAAAAAAAAAAA
CLIC5B
wildtype = BC035968
Alternative 5′ exon.
CLIC5B asv1
AAGAGCTCGTTGATTCCTCTGCAAGGTGGTGCAGCATCCTCTGTCCCTTCATTCATTTCAGATCTACTCAGGTCT
CCCTGTAAACAGATCTCTCGGATCAATAAGCATGAATGACGAAGACTACAGCACCATCTATGACACAATCCAAAA
TGAGAGGACGTATGAGGTTCCAGACCAGCCAGAAGAAAATGAAAGTCCCCATTATGATGATGTCCATGAGTACTT
AAGGCCAGAAAATGATTTATATGCCACTCAGCTGAATACCCATGAGTATGATTTTGTGTCAGTCTATACCATTAA
GGGTGAAGAGACCAGCTTGGCCTCTGTCCAGTCAGAAGACAGAGGCTACCTCCTGCCTGATGAGATATACTCTGA
ACTCCAGGAGGCTCATCCAGGTGAGCCCCAGGAGGACAGGGGCATCTCAATGGAAGGGTTATATTCATCAACCCA
GGACCAGCAACTCTGCGCAGCAGAACTCCAGGAGAATGGGAGTGTGATGAAGGAAGATCTGCCTTCTCCTTCAAG
CTTCACCATTCAGCACAGTAAGGCCTTCTCTACCACCAAGTATTCCTGCTATTCTGATGCTGAAGGTTTGGAAGA
AAAGGAGGGAGCTCACATGAACCCTGAGATTTACCTCTTTGTGAAGGCTGGAATCGATGGAGAAAGCATCGGCAA
CTGTCCTTTCTCTCAGCGCCTCTTCATGATCCTCTGGCTGAAAGG
FOXH1
wildtype = NM_003923
Different 5′UTR, retained intron between exons 3 and 4.
FOXH1 asv1
GTTGAGTCAATGTGTCCCCCTCTTGTTCCTAGGGTGCGGGCTTCATGGCCTTCTCCTCCAGGAAGCTCCACCTGA
TCATGTCCTGGGTGGATATCCAGCCCCCATAGTTCAGGGCCTACTAGCAGCTGCTAGATCTTGAACTCCAGGAGC
GCCCCACGCCTTGGGAGCTTGGCATGGGCTAAATACTCCCCCATTTGTTAAATGGGGTCCTGAAACCTGACCAGG
GAAGACGGGATAAAGTAGCCATGGGTCATCGCAGCCCCTTTGAAGCCGGGCCTGGCCACCCAAAGGCAACTCAGG
GGTGGAGACTGAGGCCTCAGGAGAAGCCCCCACTAGAATGCTCTCTGCCCCTCCCTTCCAGATTAACCAAAACCT
GCTAATTGTGGAAGCCCTCGGCATGCTCCCCTCCCCCACAGCCTCTTCCTCCCTTCCCTCCCCTCCCCCTTCCAT
CCGAATGATAAAGGCCCCAGCCCGCCTGCCCCAGCCCGGCCTCAGGTCCCGGCCCTGCCTTCTACACTGCCCCAC
CGCCCTGCACCCTCCACCCGGCCAGGCCCCTGCCCACGCTGTCTACCGTCCCGCATGGGGCCCTGCAGCGGCTCC
CGCCTGGGGCCCCCAGAGGCAGAGTCGCCCTCCCAGCCCCCTAAGAGGAGGAAGAAGAGGTACCTGCGACATGAC
AAGCCCCCCTACACCTACTTGGCCATGATCGCCTTGGTGATTCAGGCCGCTCCCTCCCGCAGACTGAAGCTGGCC
CAGATCATCCGTCAGGTCCAGGCCGTGTTCCCCTTCTTCAGGGAAGACTACGAGGGCTGGAAAGACTCCATTCGC
CACAACCTTTCCTCCAACCGATGCTTCCGCAAGGTGCCCAAGGACCCTGCAAAGCCCCAGGCCAAGGGCAACTTC
TGGGCGGTCGACGTGAGCCTGATCCCAGCTGAGGCGCTCCGGCTGCAGAACACCGCCCTGTGCCGGCGCTGGCAG
AACGGAGGTGCGCGTGGAGCCTTCGCCAAGGACCTGGGCCCCTACGTGCTGCACGGCCGGCCATACCGGCCGCCC
AGTCCCCCGCCACCACCCAGTGAGGGCTTCAGCATCAAGTCCCTGCTAGGAGGGTCCGGGGAGGGGGCACCCTGG
CCGGGGCTAGCTCCACAGAGCAGCCCAGTTCCTGCAGGCACAGGGAACAGTGGGGAGGAGGCGGTGCCCACCCCA
CCCCTTCCCTCTTCTGAGAGGCCTCTGTGGCCCCTCTGCCCCCTTCCTGGCCCCACGAGAGTGGAGGGGGAGACT
GTGCAGGGGGGAGCCATCGGGCCCTCAACCCTCTCCCCAGAGCCTAGGGCCTGGCCTCTCCACTTACTGCAGGGC
ACCGCAGTTCCTGGGGGACGGTCCAGCGGGGGACACAGGGCCTCCCTCTGGGGGCAGCTGCCCACCTCCTACTTG
CCTATCTACACTCCCAATGTGGTAATGCCCTTGGCACCACCACCCACCTCCTGTCCCCAGTGTCCGTCAACCAGC
CCTGCCTACTGGGGGGTGGCCCCTGAAACCCGAGGGCCCCCAGGGCTGCTCTGCGATCTA
SMARCC2
wildtype = BC013045
SWI/SNF related, matrix associated, actin dependent regulator of chromatin,
subfamily c, member 2
Exon 11 spliced out
SMARCC2 asv1
TGTCTTGGCTGACACACCATCAGGGCTGGTGCCTCTGCAGCCCAAGACACCTCAGCAGACCTCTGCTTCCCAACA
AATGCTCAACTTTCCTGACAAAGGCAAAGAGAAACCAACAGACATGCAAAACTTTGGGCTGCGCACAGACATGTA
CACAAAAAAGAATGTTCCCTCCAAGAGCAA
Mic
wildtype = AF143536
Cryptic splicing in exon IX
mic1 asv1
TCAGTTCCTGCAGTACCACGTCCTCAGCGACTCCAAACCTTTGGCTTGTCTGCTGTTATCCCTAGAGAGTTTCTA
TCCTCCTGCTCATCAGCTATCTCTGGACATGCTGAAGCGACTTTCAACAGCAAATGATGAAATAGTAGAAGTTCT
CCTTTCCAAACACCAAGTGTTAGCTGCCT
PC-1
wildtype = S82081
Alternative exon I, additional exon between exons 3 and 4
pc1 asv1
GAAAATGCTGGCACCTGGGCCCAGAAGCCAGGGCCTCTAACTCCTGGGGTTGATTTCTTCAGTGAAGTTGCACCT
TACAAAGGGAATATGGCCAAAGCGGCACTCAACTGAAGGCTGATATCAGGCGATTAGACAGCCATGCATTCTGCG
TTTGTCTGGAATGGATTGTAGAGAGATGGACTTATATGAGGACTACCAGTCCCCGTTTGATTTTGATGCAGGAGT
GAACAAAAGCTATCTCTACTTGTCTCCTAGTGGAAATTCATCTCCACCCGGATCACCTACTCTTCAGAAATTTGG
TCTGCTGAGAACAGACCCAGTCCCTGAGGAAGGAGAAGAGAACTTGCAAAGGTAGAAGAAGAAATCCAGACTCTG
TCTCAAGTGTTAGCAGCAAAAGAGAAGCATCTAGCAGAGATCAAGCGGAAACTTGGAATCAATTCTCTACAGGAA
CTAAAACAGAACATTGCCAAAGGGTGGCAAGACGTGACAGCAACATCTGCGAGGAGCAAGCTTCTAGCAGCAGAA
ACCGAACTGCTCTGTCTTCTGTATTGAGAGCCATCTGCAGAGCTGTTACAAGAAGACATCTGAAACCTTATCCCA
GGCTGGACAGAAGGCCTCAGCTGCTTTTTCGTCTGTTGGCTCAGTCATCACCAAAAAGCT
SF3B2
wildtype = NM_006842
Cryptic splicing in exons IX and X, deletion of 158 bp
SF3B2 asv1
GAGGAAATGGAAACAGATGCTCGCTCGTCCCGTGGCTCTGATTCCCCAGCAGCTGATGTTGAGATTGAGTATGTG
ACTGAAGAACCTGAAATTTACGAGCCCAACTTTATCTTCTTTAAG
DDX38
wildtype = NM_014003
Exon skipping, exons 3, 4, 5 and part of exon 6 deleted; deletion of 746 bp
ddx38 asv1
ATGTCTTCAAGGCTCCTGCTCCCCGCCCTTCATTACTGGGACTGGACTTGCTGGCTTCCCTGAAACGGAGAGAGC
GGCAGCAGTGGGAAGATGACCAGAGGCAAGCCGATCGGGATTGGTACATGATGGACGAGGGCTATGACGAGTTCC
ACAACCCGCTGGCCTACTCCTCCGAGGACT
CBX3
wildtype = NM_007276
Cryptic splicing in exon 4 (□81 bp), inframe splicing altered protein.
cbx3 asv1
GGGAAAAAAACAGAATGGAAAGAGTAAAAAAGTTGAAGAGGCAGAGCCTGAAGAATTTGTCGTGGAAAAAGTACT
AGATCGACGTGTAGTGAATGGGAAAGTGGAATATTTCCTGAAGTGGAAGGGAAAGCTGGCAAAGAAAAAGATGGT
ACAAAAAGAAAATCTTTATCTGACAGTGAATCTGATGACAGCAAATCAAAGAAGAAAAGAGATGCTGCTGACAAA
CCAAGAGGATTTGCC
SMARCB1
wildtype = NM_003073
Cryptic splicing in exon IV, deletion of 27 bp
SMARCB1 asv1
TCACTCTGGAGGCGACTAGCCACTGTGGAAGAGAGGAAGAAAATAGTTGCATCGTCACATGATCACGGATACACG
ACTCTAGCCACCAGTGTGACCCTGTTAAAAGCCTCGGAAGTGGAAGAGATTCTGGATGGCAACGATGAGAAGTAC
AAGGCTGTGTCCATCAGCACAGAGCCCCCC
SMARCC1
wildtype = NM_003074
Exon skipping, exon 18 deleted, deletion of 111 bp
SMARCC1 asv1
GGAAAGTAGACCCATGGCAATGGGACCTCCTCCTACTCCTCATTTTAATGTATTAGCTGATACCCCCTCTGGGCT
TGTGCCTCTGCATCTTCGATCACCTCAGAGTAAGGTGCTAGTGCTGGAAGAGAATGGACTGAACAGGAGACCCTT
CTACTCCTGGAGGCCCTGGAGATGTACAA
SMARCA5
wildtype = BU600776
Exon skipping, exons 8, 9 and 10 deleted; deletion of 420 bp
smarca5 asv1
AAGCCTCGAATGGGCGAAAGTTCACTTAGAAACTTTACAATAGATCTGTTTGTTTGATAGGAGATAAAGAACAAA
GAGCTGCTTTTGTCAGAGACGTTTTATTACCGGGAGAATGGTATACTCGGATATTAATGAAGGATATAGATATAC
TCAACTCAGCAGGCAAGATGGACAAAATGAGGTTATTGAACATCCTAATGCAGTTGAGAA
DNAJC8
wildtype = NM_014280
Alternative exon 2
DNAJC8 asv1
AGAGAGCGGGACTTCAGGCGGCGGAGGCAGCACCGAGGAAGCATTTATGACCTTCTACAGTGAGGAATAAAGATG
GCATATAGCATACCAGAGATTCATTCCAACTAGCATTCCAACTCTGACAGTGACACCAAGAATGTTTTCCTGGGA
CTGCCTGGTGCTTGTTCTCCCTGGCATTGTCTTCAGGTGAAACAAATAGAGAAGAGAGACTCGGTTCTAACTTCG
AAAAATCAGATTGAAAGACTGACCCGTCCTGGTTCCTCTTACTTCAATTTGAACCCATTTGAGGTTCTTCAGATA
SFRS7
wildtype = NM_006276
Exon skipping, exon 7 deleted
SFRS7 asv1
GAGGTATTTCCAATCCCCGTCGAGGTCAAGATCAAGATCCAGGTCTATTTCACGACCAAGAAGCAGTCGTTCCCC
ATCAGGAAGTCCTCGCAGAAGTGCAAGTCCTGANAAGAATGGACTGAAAGCTTCTCAGTTCACCCTTTTAGGGGA
AAAGTTATTTTTGGTTACATTATTATAAAG
SFRS9
wildtype = NM_003769
Exon 3 uses cryptic splice site, deletion of 40 bp in exon 3
sfrs9 asv1
GCAGCTGGCAGGACCTGAAGGATCACATGCGAGAAGCTGGGGATGTCTGTTATGCTGATGTGCAGAAGGATGGAG
TGGGGATGGTCGAGTATCTCAGAAAAGAAGACATGAGGGTGAAACTTCCTACATCCGAGTTTATCCTGAGAGAAG
CACCAGCTATGGCTACTCACGGTCTCGGTC
PRP19
wildtype = AJ131186
Exon skipping, exons 2-12 deleted, deletion of 1495 bp
prp19 asv1
TTGTTTTCTTTTTTTAATGAAACTAGATCACTGCTTACAAAACCCTGCACAAGCCCTCCTGCCCATCCCCTTCAC
AGTTCCCTTGGTGAGACGGGCAATGACACGGCAAGCGGCATCGTGCTGGTACAGAGCGTGTGACAGCTCTTGGCG
GGTTGTCTGCAGCTGCTGGCGCAGAGTGAA
GTF3C5
wildtype = NM_012087
deleted (exon IV partly + exonV entirely, deletion of 199 bp) + additional
exon VIII (insertion of 20 bp)
gtf3c5 asv1
CCCCCCATCTCAGGTGAGAATCTGATTGGCCTGAGCAGAGCCCGGCGCCCCCACAATGCCATCTTTGTCAACTTT
GAGGATGAGGAGGTGCCCAAGCAGCCTATGGATTCGATTTGGGTATGACCCCCGGAAAAACCCAGATGCCAAGAT
TTATCAAGTCCTCGATTTCCGAATCCGTTGTGGAATGAAACACGGTTACGCCCCCAGTGACTTGCCGGTCAAAGC
AAAGCGCAGCACCTACAACTACAGCCTCCCCATCACCGTCAAGAAGACATCCAGCCAGCTTGTCACCATGCATGA
CCTGAAGCAGGGCCTGGGCCCGTCGGGGACGAGTGGTGCTCGGAAACCAGCTTCCAGCAAGTACAAGCTCAAGGT
CAGCCTTCAGACACTGAGGGACTCTGTCTACATCTTCCGGGAAGGGGCCTTGCCACCCTATCGGCAGATGTTCTA
CCAGTTATGCGACTTGAATGTGGAAGAGTT
LISCH7
wildtype = AK126834
Exon 4 spliced out; deletion of 146 nucleotides
lisch7 asv1
CGGAAATGCTGACCTGACCTTTGACCAGACGGCGTGGGGGGACAGTGGTGTGTATTACTGCTCCGTGGTCTCAGC
CCAGGACCTCCAGGGGAACAATGAGGCCTACGCAGAGCTCATCGTCCTTGTGTATGCCGCCGGCAAAGCAGCCAC
CTCAGGTGTTCCCAGCATTTATGCCCCCAGCACCTATGCCCACCTGTCTCCCGCCAAGACCCCACCCCCACCAGC
TATGATTCCCATGGG
RIPK2
wildtype = NM_003821
Exon 2 skipping, (154 nucleotides), usage of downstream ATG
RIPK2 asv1
TCCGCCCGCCACGCAGACTGGCGCGTCCAGGTGGCCGTGAAGCACCTGCACATCCACACTCCGCTGCTCGACAGA
AAACTGAATATCCTGATGTTGCTTGGCCATTGAGATTTCGCATCCTGCATGAAATTGCCCTTGGTGTAAATTACC
neogenin1
wildtype = U61262
Exon 21 spliced out; deletion of 33 nucleotides
neogenin1 asv1
GACTCACCAGATACAAGAGTTAACTCTTGACACACCATACTACTTCAAAATCCAGGCACGGAACTCAAAGGGCAT
GGGACCCATGTCTGAAGCTGTCCAATTCAGAACACCTAAAGCCTCAGGGTCTGGAGGGAAAGGAAGCCGGCTGCC
AGACCTAGGATCCGACTACAAACCTCCAATGAGCGGCAGTAACAGCCCTCATGGGAGCCCCACCTCTCCTCTGGA
CAGTAATATGCTGCTGGTCATAATTGTTTCTGTTGGCGTCATCACCATCGTGGTGGTTGTGATTATCGCTGTCTT
ADRM1
wildtype = NM_175573
Exon 3 cryptic splicing; deletion of 92 bp
adrm1 asv1
GCAGACGGACGACTCGCTTATTCACTTCTGCTGGAAGGACAGGACGTCCGGGAACGTGGAAGACGACTTGATCAT
CTTCCCTGACGACTGAACCCAAGACAGACCAGGATGAGGAGCATTGCCGGAAAGTCAACGAGTATCTGAACAACC
CCCCGATGCCTGGGGCGCTGGGGGCCAGCGGAAGCAGCGGCCACGAACTCTCTGCGCTAGGCGGTGAGGGTGGCC
KLF5
wildtype = AF132818
Additional exon after exon 3; insertion of 59 nucleotides
klf5 asv1
AAGTTTATACCAAGTCTTCTCATTTAAAAGCTCACCTGAGGACTCACACTGTGTGAAGTTATCAGTACCAGACTA
TTTTGCTTCAATCTGCAAAAGGAAGGTGTGTGAAGGTGAAAAGCCATACAAGTGTACCTGGGAAGGCTGCGACTG
GAGGTTCGCGCGATCGGATGAGCTGACCCG
Bid
wildtype = NM_001196
exon 3 skipping (70 nucleotides), translation initiation of downstream ATG
as compared to NM_001196
Bid asv1
CCGCGCGCCTGGGAGACGCTGCCTCGGCCCGGACGCGCCCGCGCCCCCGCGGCTGGAGGGTGGTCAACAACGGTT
CCAGCCTCAGGGATGAGTGCATCACAAACCTACTGGTGTTTGGCTTCCTCCAAAGCTGTTCTGACAACAGCTTCC
Bax
wildtype = NM_138761
An extra exon (98 bp) inserted between exons 4 and 5
Bax asv1
AGTGGCAGCTGACATGTTTTCTGACGGCAACTTCAACTGGGGCCGGGTTGTCGCCCTTTTCTACTTTGCCAGCAA
ACTGGTGCTCAAGGCTGGCGTGAAATGGCGTGATCTGGGCTCACTGCAACCTCTGCCTCCTGGGTTCAAGCGATT
CACCTGCCTCAGCATCCCAAGGAGCTGGGATTACAGGCCCTGTGCACCAAGGTGCCGGAACTGATCAGAACCATC
ATGGGCTGGACATTGGACTTCCTCC
CASP9
wildtype = NM_001229
skipping of exons 3, 4, 5, 6 (450 nucleotides)
CASP9 asv1
ACCAGAGGTTCTCAGACCGGAAACACCCAGACCAGTGGACATTGGTTCTGGAGGATTTGGTGATGTCGAGCAGAA
AGACCATGGGTTTGAGGTGGCCTCCACTTCCCCTGAAGACGAGTCCCCTGGCAGTAACCCCGAGCCAGATGCCAC
Bak
wildtype = NM_001188
An extra exon (20 bp) between exons 4 and 5
Bak asv1
TGCAGCACCTGCAGCCCACGGCAGAGAATGCCTATGAGTACTTCACCAAGATTGCCACCAGGCCAGCAGCAACAC
CCACAGCCTGTTTGAGAGTGGCATCAATTGGGGCCGTGTGGTGGCTCTTCTGGGCTTCGGCTACCGTCTGGCCCT
BCL2L1
wildtype = NM_138578
Skipping of 3′ part of exon 1(189 nucleotides)
BCL2L1 asv1
CTGCGGTACCGGCGGGCATTCAGTGACCTGACATCCCAGCTCCACATCACCCCAGGGACAGCATATCAGAGCTTT
GAACAGGATACTTTTGTGGAACTCTATGGGAACAATGCAGCAGCCGAGAGCCGAAAGGGCCAGGAACGCTTCAAC
CG
Casp2
wildtype = NM_032982
skipping of part of exon 3, exon 4 entirely and part of exon 5 (218
nucleotides)
Casp2 asv1
GGAAATGAGGGAGCTCATCCAGGCCAAAGTGGGCAGTTTCAGCCAGAATGTGGAACTCCTCAACTTGCTGCCTAA
GAGGGGTCCCCAAGCTTTTGATGCCTTCTGTGAAGCCTTGCACTCCTGAATTTTATCAAACACACTTCCAGCTGG
CATATAGGTTGCAGTCTCGGCCTCGTGGCCTAGCACTGGTGTTGAGCAAT
SUMF2
wildtype = BC006159
Exon 4 spliced out; deletion of 46 nucleotides
sumf2 asv1
AGAAGCTGAGATGTTTGGATGGAGCTTTGTCTTTGAGGACTTTGTCTCTGATGAGCTGAGAAACAAAGCCACCCA
GCCAATGAGCCTGCAGGTCCTGGCTCTGGCATCCGAGAGAGACTGGAGCACCCAGTGTTACACGTGAGCTGGAAT
GACGCCCGTGCCTACTGTGCTTGGCGGGGA
G2AN
wildtype = NM_198335
Exon 6 is spliced out, exon 7 uses different splice acceptor.
G2AN asv1
GTCTTTTGCTTAGTGTCAATGCCCGAGGACTCTTGGAGTTTGAGCATCAGAGGGCCCCTAGGGTCTCCCCCTCGT
CCCTGCCCCCTCTGGATTGGAGCAGACAGCTCTCCTACCTTCCAGGCAAGGATCAAAAGACCCAGCTGAGGGCGA
TGGGGCCCAGCCTGAGGAAACACCCAGGGATGGCGACAAGCCAGAGGAGACTCAGGGGAA
HCCR1
wildtype = AF195651
Exons 3-6 spliced out; deletion of 488 nucleotides
HCCR1 asv1
CTTATGTGGTAACCAAGACAAAAGCGATTAATGGGAAATACCATCGTTTCTTGGGTCGTCATTTCCCCCGCTTCT
ATATCCTGTACACAATCTTCATGAAAGAAAGCCTTGAGCCGGGCCATGCTTCTCACATCTTACCTGCCTCCTCCC
TTGTTGAGACATCGTTTGAAGACTCATACA
asns
wildtype = AK000379
Alternative splice acceptor in exon 4, leading to an extended exon;
insertion of 74 nucleotides
asns asv1
TCTGGAGAAGGATCAGATGAACTTACGCAGGGTTACATATATTTTCACAAGGATTGGAGAGGGAGAAAGAAAAAC
TGCTTTGTGTGCCAAAAGCAAAACTCTTGGTGTTTTTGTTTGTGAAATAGGCTCCTTCTCCTGAAAAAGCCGAGG
AGGAGAGTGAGAGGCTTCTGAGGGAACTCTATTTGTTTGATGTTCTCCGCGCAGATCGAACTACTGCTGCCCATG
GTCTTGAACTGAGAG
HSACP1
wildtype = BC007422
Additional exon inserted after exon 2; insertion of 29 nucleotides
HSACP1 asv1
ATGGCGGAACAGGCTACCAAGTCCGTGCTGTTTGTGTGTCTGGGTAACATTTGTCGATCACCCATTGCAGAAGCA
GTTTTCAGGAAACTTGTAACCGATCAAAACATCTCAGAGAATTGGAGGGTAGACAGCGCGGCAACTTCCGGTGGG
TCATTGATAGCGGTGCTGTTTCTGACTGGAACGTGGGCCGGTCCCCAGACCAAGAGCTGTGGAGCTGCCTAAGAA
ATCATGGCATTCACACAGCCCATAAAGCAAGACAGATTACCAAAGAAGATTTTGCCACATTTGATTATATACTAT
GTATGGATGAAAGCAATCTGAGAGATTTGAATAGAAAAAGTAATCAAGTTAAAACCTGCAAAGCTAAAATTGAAC
TACTTGGGAGCTATGATCCACAAAAACAACTTATTATTGAAGATCCCTATTATGGGAATGACTCTGACTTTGAGA
CGGTGTACCAGCAGTGTGTCAGGTGCTGCAGAGCGTTCTTGGAGAAGGCCCACTGAGGCAGGTTCGTGCCCTGCT
GCGGCCAGCCTGACTAGACCCCACCCTGAGGTCCTGCATTTCTCAGTCGGTG
CREB3L4
wildtype = BC038962
Exon 2 uses a cryptic splice donor, leading to a smaller exon; deletion of
60 nucleotides
CREB3L4 asv1
CTGGCAAGAAGCATGGATCTCGGAATCCCTGACCTGCTGGACGCGTGGCTGGAGCCCCCAGAGGATATCTTCTCG
ACAGGATCCGTCCTGGAGCTGGGACTCCACTGCCCCCCTCCAGAGGTTCCGGGCCTTCAAGAGAGTGAGCCTGAA
GATTTCTTGAAGCTTTTCATTGATCCCAATGAGGTGTACTGCTCAGAAGCATCTCCTGGCAGTGACAGTGGCATC
TCTGAGGACCCCTGC
Hes6
wildtype = BC007939
Exon 2 spliced out; deletion of 87 nucleotides
hes6 asv1
GGGCATGGCGCCACCCGCGGCGCCTGGCCGGGACCGTGTGGGCCGTGAGGATGAGGACGGCTGGGAGACGCGAGG
GGACCGCAAGGTGCAGGCCAAGCTGGAGAACGCCGAAGTGCTGGAGCTGACGGTGCGGCGGGTCCAGGGTGTGCT
GCGGGGCCGGGCGCGCGAGCGCGAGCAGCT
C20orf45
wildtype = BC013969
Exon 3 spliced out; deletion of 90 nucleotides
C20orf45 asv1
GGTTGGAGTTGATGTGTTGGACAGACATATAGATCCCTCTGGAAAGTTGCACAGCCACAGACTTCTCAGCACAGA
GTGGGGACTGCCTTCCATTGTGAAGTCTATTTCATTTACAAACATGGTTTCAGTAGATGAGAGACTTATATACAA
ACCACATCCTCAGGATCCAGAAAAAACTGT
macropain
wildtype = BC047897
Exons 6-17 spliced out; deletion of 1138 nucleotides
macropain asv1
CTAAAAAACACAAAGGATGCAGTACGGAATTCTGTATGTCATACTGCAACCGTTATAGCAAACTCTTTTATGCAC
TGTGGGACAACCAGTGACCAGTTTCTTAGAGATAATTTGGTTCTGGTTTCCTCTTTCACACTTCCTGTCATTGGC
TTATACCCCTACCTGTGTCATTGGCCTTAA
SPI2
wildtype = BC012868
Exon 2 spliced out; deletion of 170 nucleotides
SPI2 asv1
GCGTTTCTCGCCCTGCTGGGATCGCTGCTCCTCTCTGGGGTCCTGGCGGCCGACCGAGAACGCAGCATCCACGAG
AATGCCACGGGTGACCTGGCCACCAGCAGGAATGCAGCGGATTCCTCTGTCCCAAGTGCTCCCAGAAGGCAGGAT
TCTGAAGACCACTCCAGCGATATGTTCAACTATGAAGAATACTGCACCGCCAACGCAGTC
TCOF1
wildtype = U40847
Exon 21 spliced out; deletion of 114 nucleotides
TCOF1 asv1
AGTCGGATATCAGATGGCAAGAAACAGGAGGGACCAGCCACTCAGGTTGACAGTGCTGTGGGAACACTCCCTGCA
ACAAGTCCCCAGAGCACCTCCGTCCAGGCCAAAGGGACCAACAAG
CIB1
wildtype = NM_006384
Difference in 3′UTR (intron insertion)
cib1 asv1
CGTTCTCCAGACTTTGCCAGCTCCTTTAAGATTGTCCTGTGACAGCAGCCCCAGCGTGTGTCCTGGCACCCTGTC
CAAGAACCTTTCTACTGCTGGCCCAGCCTGGAGCTGGCGCTGTGCAGCCTCACCCCGGGCAGGGGCGGCCCTCGT
TGTCAGGGCCTCTCCTCACTGCTGTTGTCATTGCTCCGTTTGTGTTTGTACTAATCAGTAATAAAGGTTTAGAAG
TROAP
wildtype = NM_005480
Intron insertion in front of the last exon.
troap asv1
AGGAACAGCTTGAAGTACCAGAGCCCTACCCTCCAGCAGAACCCAGGCCCCTAGAGTCCTGCTGTAGGAGTGAGC
CTGAGATACCGGAGTCCTCTCGCCAGGAACAGCTTGAGGAACAGCTTGAGGTACCTGAGCCCTGCCCTCCAGCAG
AACCCGGGCCCCTTCAGCCCAGCACCCAGGGGCAGTCTGGACCCCCAGGGCCCTGCCCTAGGGTAGAGCTGGGGG
TROAP
wildtype = NM_005480
Cryptic splicing in exon III, exon III shorter for 91 bp
troap asv2
CCGTGGACCAGGAGAACCAAGATCCAAGGAGATGGGTGCAGAAACCACCGCTCAATATTCAACGCCCCCTCGTTG
ATTCAGCAGGCCCCAGGCCGAAAGCCAGGCACCAGGCAGAGACATCACAAAGATTGAGGCTCCAGGGACCATAGA
GTTTGTGGCTGACCCTGCAGCCCTGGCCACCATCCTGTCAGGTGAGGGTGTGAAGAGCTGTCACCTGGGGCGCCA
PARVA
wildtype = NM_018222.2
Exon 8 skipping
parva asv1
AACGAGAAGGAATCCTCCAGTCTCGGCAAATCCAAGAGGAAATAACTGGTAACACAGAAACGTGATGCCTTTGAC
ACCTTGTTCGACCATGCCCCAGACAAGCTGAATGTGGTGAAAAAGACACTCATCACTTTCGTGAACAAGCACCTG
ILK
wildtype = U40282
Additional exon (exon 3a)
ilk asv1
GCTGCTATGGACGACATTTTCACTCAGTGCCGGGAGGGCAACGCAGTCGCCGTTCGCCTGTGGCTGGACAACACG
GAGAACGACCTCAACCAGGGTATCGTCTTGGATGCTTTGTGAAGAGCAGGTGGAAAGGAGGCAATTGCCTAGTTC
ATCGTAGAAGTAATGATGTCTTGGACTAGAATTAGGGGACGATCATGGCTTCTCCCCCTTGCACTGGGCCTGCCG
AGAGGGCCGCTCTGCTGTGGTTGAGATGTTGATCATGCGGGGGGCACGGATCAATGTAATGAACCGTGGGGATGA
ILK
wildtype = U40282
Introns 6 and 7 retained
ilk asv2
CGAGAGCGGGCAGAGAAGATGGGCCAGAATCTCAACCGTATTCCATACAAGGACACATTCTGGAAGGGGACCACC
CGCACTCGGCCCCGTGAGTCACCACTGTGGGAAGAAGGGTTGTAAAAGGAAATAATCCTGGCCTCTTGGGGCTGG
GTTAGGGTGAAGCTGGGTACCTGACCTGCCCACACTCTTAGGAAATGGAACCCTGAACAAACACTCTGGCATTGA
CTTCAAACAGCTTAACTTCCTGACGAAGCTCAACGAGAATCACTCTGGAGAGGTGACCCCTGCCCTTCTTGCCCT
TCCCTCACTAAACCCCCATAAATTACTTGCTTTGTACCTGTTTTAAGTTTTTCCTCCAGTTAGTGGGCAAGGAAG
TGGCAGCAACATTTCAAGCCTCCTAACCCCTACCTGTCCTGCAGCTATGGAAGGGCCGCTGGCAGGGCAATGACA
TTGTCGTGAAGGTGCTGAAGGTTCGAGACTGGAGTACAAGGAAGAGCAGGGACTTCAATGAAGAGTGTCCCCGGC
ITGA7
wildtype = AF052050
Intron 16 retained.
itga7 asv1
CCCCAGGCTGATGGGGATGATGCCCATGAAGCCCAGCTCCTGGTCATGCTTCCTGACTCACTGCACTACTCAGGG
GTCCGGGCCCTGGACCCTGCGGTGAGGACCTGGGGGCAGGATGGGGTGGGGTCTTGAGGGGCTCCAGTAACCCAG
ACTGACCTTGCCTTCTCTCCCATTCCAGGAGAAGCCACTCTGCCTGTCCAATGAGAATGCCTCCCATGTTGAGTG
TGAGCTGGGGAACCCCATGAAGAGAGGTGCCCAGGTCACCTTCTACCTCATCCTTAGCACCTCTGGGATCAGCAT
ITGA5
wildtype = NM_002213.3
Exon 8 deleted
itga5 asv1
CTGAACGAGGCCAACGAGTACACTGCATCCAACCAGATGGACTATCCATCCCTTGCCTTGCTTGGAGAGAAATTG
GCAGAGAACAACATCAACCTCATCTTTGCAGTGACAAAAAACCATTATATGCTGTACAAGAGTATCCGGTCTAAA
GTGGAGTTGTCAGTCTGGGATCAGCCTGAGGATCTTAATCTCTTCTTTACTGCTACCTGCCAAGATGGGGTATCC
NCAM
wildtype = BC047244
Exons 17 and 18 deleted
ncam asv1
CAGGCAGAATATTGTGAATGCCACCGCCAACCTCGGCCAGTCCGTCACCCTGGTGTGCGATGCCGAAGGCTTCCC
AGAGCCCACCATGAGCTGGACAAA
ZD52F10
wildtype = BC011886
Alternative use of exon 2
Splicing does not change the protein.
zd52f10 asv1
GGTGAAGTTTTGGTAGGTGAGTGTCAGAGTGAGCCGACCCAGGCCACATCCTGGCAGTGGAGGCACAGTCACCCG
GGGCAGGGCCAGGATCTTGGTATATCCTCAGATCTCAGTGGGCAGCGACATGAAGTCAGGCAATTTCTTGCAACC
ACCACCGAGGCCCCGAAAAGCACTGGTCGTCAGGGAGCTCCTCCCCTTGGCCCCCAGCCTGTGCCAGCCCTGGCC
CGGCTGCCACACCTC
Diablo
wildtype = NM_019887
Alternative exon 2 and exon 3 (132 bp) skipping
DIABLO asv1
GATAGCGTCTGGCGTCCGCGCGCTGCACAATGGCGGCTCTGAAGAGTTGGCTGTCGCGCAGCGTAACTTCATTCT
TCAGGTTCCTGCTTGGCTCGAGTTTGAGTTTACAGCCCCTGCAAGTAAATCCAAGAGCCTGTTACAGATTGGCGG
TCGTGCCTTATGAAATCTGACTTCTACTTCCAGGCTGTTTATACCTTAACTTCTCTTTACCGACAATATACAAGT
TTACTTGGGAAAATGAATTCAGAGG
CASP8
wildtype = NM_001228
Exon 4 (96 bp) and exon 8 skipping (not shown), exon 7 inclusion (47 bp)
CASP8 asv1
GAAAGGAGGAGATGGAAAGGGAACTTCAGACACCAGGCAGGGCTCAAATTTCTGCCTACAGGGTCATGCTCTATC
AGATTTCAGAAGAAGTGAGCAGATCAGAATTGAGGTCTTTTAAGTTTCTTTTGCAAGAGGAAATCTCCAAATGCA
AACTGGATGATGACATGAACCTGCTGGATATTTTCATAGAGATGGAGAAGAGGGTCATCCTGGGAGAAGGAAAGT
TGGACATCCTGAAAAGAGTCTGTGCCCAAATCAACAAGAGCCTGCTGAAGATAATCAACGACTATGAAGAATTCA
GCAAAGAGAGAAGCAGCAGCCTTGAAGGAAGTCCTGATGAATTTTCAAATGACTTTGGACAAAGTTTACCAAATG
AAAAGCAAACCTCGGGGATACTGTCTGATCATCAACAATCACAATTTTGCAAAAGCACGGGAGAAAGTGCCCAAA
Casp3
wildtype = NM_004346
Exon2 (UTR) skipping, exon 7 (121 bp) skipping
Casp3 asv1
AGTGCAGACGCGGCTCCTAGCGGATGGGTGCTATTGTGAGGCGGTTGTAGAAGTTAATAAAGGTATCCATGGAGA
ACACTGAAAACTCAGTGGATTCAAAATCCATTAAAAATTTGGAACCAAAGATCATACATGGAAGCGAATCAATGG
ACTCTGGAATATCCCTGGACAACAGTTATAAAATGGATTATCCTGAGATGGGTTTATGTATAATAATTAATAATA
AGAATTTTCATAAAAGCACTGGAATGACATCTCGGTCTGGTACAGATGTCGATGCAGCAAACCTCAGGGAAACAT
TCAGAAACTTGAAATATGAAGTCAGGAATAAAAATGATCTTACACGTGAAGAAATTGTGGAATTGATGCGTGATG
TTTCTAAAGAAGATCACAGCAAAAGGAGCAGTTTTGTTTGTGTGCTTCTGAGCCATGGTGAAGAAGGAATAATTT
TTGGAACAAATGGACCTGTTGACCTGAAAAAAATAACAAACTTTTTCAGAGGGGATCGTTGTAGAAGTCTAACTG
GAAAACCCAAACTTTTCATTATTCAGGTTATTATTCTTGGCGAAATTCAAAGGATGGCTCCTGGTTCATCCAGTC
GCTTTGTGCCATGCTGAAACAGTATGCCGACAAGCTTGAATTTATGCACA
RON
wildtype = NM_002447
Exon 5, exon 6 and exon 11 deleted (534 bp)
RON asv1
ATGTGCGGCCAGCAGAAGGAGTGTCCTGGCTCCTGGCAACAGGACCACTGCCCACCTAAGCTTACTGAGGAGCCA
GTGCTGATAGCAGTGCAACCCCTCTTTGGCCCACGGGCAGGAGGCACCTGTCTCACTCTTGAAGGCCAGAGTCTG
TCTGTAGGCACCAGCCGGGCTGTGCTGGTCAATGGGACTGAGTGTCTGCTAGCACGGGTCAGTGAGGGGCAGCTT
TTATGTGCCACACCCCCTGGGGCCACGGTGGCCAGTGTCCCCCTTAGCCTGCAGGTGGGGGGTGCCCAGGTACCT
GGTTCCTGGACCTTCCAGTACAGAGAAGACCCTGTCGTGCTAAGCATCAGCCCCAACTGTGGCTACATCAACTCC
CACATCACCATCTGTGGCCAGCATCTAACTTCAGCATGGCACTTAGTGCTGTCATTCCATGACGGGCTTAGGGCA
GTGGAAAGCAGGTGTGAGAGGCAGCTTCCAGAGCAGCAGCTGTGCCGCCTTCCTGAATATGTGGTCCGAGACCCC
CAGGGATGGGTGGCAGGGAATCTGAGTGCCCGAGGGGATGGAGCTGCTGGCTTTACACTGCCTGGCTTTCGCTTC
CTACCCCCACCCCATCCACCCAGTGCCAACCTAGTTCCACTGAAGCCTGAGGAGCATGCCATTAAGTTTGAGGTC
TGCGTAGATGGTGAATGTCATATCCTGGGTAGAGTGGTGCGGCCAGGGCCAGATGGGGTCCCACAGAGCACGCTC
AR
wildtype = NM_000044
Skipping of exon 2, exon 3 and exon 4 (557 bp)
AR asv1
GCCCTATCCCAGTCCCACTTGTGTCAAAAGCGAAATGGGCCCCTGGATGGATAGCTACTCCGGACCTTACGGGGA
CATGCGGCTTCCGCAACTTACACGTGGACGACCAGATGGCTGTCATTCAGTACTCCTGGATGGGGCTCATGGTGT
CD82
wildtype = NM_002231
Skipping of exon 9 (84 bp)
CD82 asv1
GGGCTTCTGCGAGGCCCCCGGCAACAGGACCCAGAGTGGCAACCACCCTGAGGACTGGCCTGTGTACCAGGAGCT
CCTGGGGATGGTCCTGTCCATCTGCTTGTGCCGGCACGTCCATTCCGAAGACTACAGCAAGGTCCCCAAGTACTG
MUC2
wildtype = NM_002457
Skipping of 3′ part of Exon 30(ca 7200 nucleotides, ORF remains)
MUC2 asv1
TGGGGTCATCCCTATGGCCTTCTGCCTCAACTACGAGATCAACGTTCAGTGCTGCACCCCCACTCGCGGTACCAC
GACCGGGTCATCTTCAGCCCCCACCCCCAGCACTGTGCAGACGACCACCACCAGTGCCTGGACCCCAACGCCGAC
RIOK1
wildtype = NM_031480
Cryptic splicing of exon 3 (insertion of 32 bp)
RIOK1 asv1
TTGGAAAACTCGCCAAGGGTTATGTCTGGAATGGAGGAAGCAACCCACAGCTAGTGCCTTAGACTCTGGAATTCC
CTTCTAGGCAAATCGACAGACCTCCGACAGCAGTTCAGCCAAAATGTCTACTCCAGCAGACAAGGTCTTACGGAA
RHAMM
wildtype = NM_012484
Hyaluronan-mediated motility receptor
Exon 4 skipping (45 bp)
RHAMM asv1
TGTTGACAAAGATACTACCTTGCCTGCTTCAGCTAGAAAAGTTAAGTCTTCGGAATCAAAGATTCGTGTTCTTCT
ACAGGAACGTGGTGCCCAGGACAGCCGGATCCAGGATCTGGAAACTGAGTTGGAAAAGATGGAAGCAAGGCTAAA
DDR1a
wildtype = NM_013993
Alternative 5′ exons and skipping of exon 11 (111 bp)
DDR1 asv1
CGTGGGAATCCGCCCCACTCCGCTCCCTGTGTCCCCAATGGCTCTGCCTACAGTGGGGACTATATGGAGCCTGAG
AAGCCAGGCGCCCCGCTTCTGCCCCCACCTCCCCAGAACAGCGTCCCCCATTATGCCGAGGCTGACATTGTTACC
TNFRSF10B
wildtype = NM_003842
Cryptic intron in exon 5 spliced out (87 bp)
TNFRSF10B asv1
TGCCGCACAGGGTGTCCCAGAGGGATGGTCAAGGTCGGTGATTGTACACCCTGGAGTGACATCGAATGTGTCCAC
AAAGAATCAGGCATCATCATAGGAGTCACAGTTGCAGCCGTAGTCTTGATTGTGGCTGTGTTTGTTTGCAAGTCT
CSE1L
wildtype = NM_001316
An extra exon (25 bp) inserted before last exon
CSE1L asv1
AACCCCAAAATTCACCTGGCACAGTCACTTCACAAGTTGTCTACCGCCTGTCCAGGAAGGACCTATTTTTGAAGG
CATAAAAGCAGTTCCATCAATGGTGAGCACCAGCCTGAATGCAGAAGCGCTCCAGTATCTCCAAGGGTACCTTCA
MLH1
wildtype = NM_000249
Exon 12 skipping (371 nucleotides)
MLH1 asv1
TTCACTTCCTGCACGAGGAGAGCATCCTGGAGCGGGTGCAGCAGCACATCGAGAGCAAGCTCCTGGGCTCCAATT
CCTCCAGGATGTACTTCACCCAGAAAGAGACATCGGGAAGATTCTGATGTGGAAATGGTGGAAGATGATTCCCGA
AAGGAAATGACTGCAGCTTGTACCCCCCGGAGAAGGATCATTAACCTCAC
MSH2
wildtype = NM_000251
Skipping of exons 2-8 (1175 nucleotides)
MSH2 asv1
GCGCACGGCGAGGACGCGCTGCTGGCCGCCCGGGAGGTGTTCAAGACCCAGGGGGTGATCAAGTACATGGGGCCG
GCAGGTGGAAAACCATGAATTCCTTGTAAAACCTTCATTTGATCCTAATCTCAGTGAATTAAGAGAAATAATGAA
CCND1
wildtype = Z23022
G to A polymorphism in the end of exon 4 results in intron 4 retention and
exon 5 skipping
ccnd1 asv1
CCTGAACCTGAGGAGCCCCAACAACTTCCTGTCCTACTACCGCCTCACACGCTTCCTCTCCAGAGTGATCAAGTG
TGACCCAGTAAGTGAGGGTGATGTCCCAGGCAGCCTTGCCGGGGCTTACAGGGGGAGACACCTAGTGCCACGGAA
ATGCCGAGGCTGGTGCCAAGGCCCCCAAGGGTGACAAGGTTGGGGCTGGGGCTGGGCCCCTCGGACCCCAGGCCA
CAGACTGACAGGGCACCGGCTTCTTCCACTGCTCCTAGAACTTACTGACTGGCTGGGAGGTCCTCACAGCCTTCT
CACGTCCCCTGGGGCTTCCAGGAGCCGTAGAGTTTCTGGGCGAAGCGTCCGGGACGGAGGCCCCAGGCGGCCCCA
GCCAATGGTCTGTGTGGTGATGGTGTGTGGGGTTAGGCCCAGGCGAGCTTTGTTTGGGCCACAATGTGCGTGGCC
AATAAATAGATGCTTGAAAAGGGCTCCTGTGAGGTCCGAGACACCGGACAACGGGCGGATAGAGACAGCCTTGTT
GTTTACGGCCTCTTTGAGAGGCTGCTGCTGTTAAACCCTGGGATGACTGTGTCTTTCTTCTTAAAAATGCCATTG
TTTTATTCCCGAGTCTTTTCTTAAAGAAAGAATTAAAATGACAATCAAAAGGGTTTGTGGCATTTACCAAATTAG
ACCAGAGAGGTGGCCGGGTCAGCCGCCGGCCCCGC
REST
wildtype = NM_005612
Inclusion of an extra exon (50 bp) ) between coding exons 2 and 3
REST asv1
TCAGAAGACTCATCTAACTAGACATATGCGTACTCATTCAGTGGGGTATGGATACCATTTGGTAATATTTACTAG
AGTGTGATCTAGATGGGTGAGAAGCCATTTAAATGTGATCAGTGCAGTTATGTGGCCTCTAATCAACATGAAGTA
GHRHR
wildtype = AF282259
Skipping of exons 2, 3, 4 (385 bp)
GHRHR asv1
TTGTACTATCACTGGCTGGTCTGAGCCCTTTCCACCTTACCCTGTGGCCTGCCCTGTGCCTCTGGAGCTGCTGGC
TGAGGAGGGCTGCCCGTGCTCTTCACTGGCACGTGGGTGAGCTGCAAACTGGCCTTCGAGGACATCGCGTGCTGG
PTPN18
wildtype = NM_014369
Skipping of 193 bp in 3′ UTR, protein sequence does not change
PTPN18 asv1
CCGAAGGGTCCCCGGGACCCGCCTGCTGAGTGGACCCGGGTGTAAGTCTAACGCCAGTTCCTGCACAGAGCAGAT
TCAAGAAAGAAGATCAGGAAGGGGCATGACCCCTGAGTTATGAAGGGGAGAAGGGACAGATGAGCTTCCGGAGAC
ASC
wildtype = NM_013258
Exon 2 skipping (57 bp)
ASC asv1
AACGTGCTGCGCGACATGGGCCTGCAGGAGATGGCCGGGCAGCTGCAGGCGGCCACGCACCAGGGCCTGCACTTT
ATAGACCAGCACCGGGCTGCGCTTATCGCGAGGGTCACAAACGTTGAGTGGCTGCTGGATGCTCTGTACGGGAAG
BCL2L12
wildtype = NM_138639
Exon 6 skipping (273 bp)
BCL2L12 asv1
GAAGCCATACTGCGGAGGCTGGTGGCCCTGCTGGAGGAGGAGGCAGAAGTCATTAACCAGAAGGAGGGCATCCTG
GCTGTTTCACCCGTGGACTTGAACTTGCCATTGGACTGAGCTCTTTCTCAGAAGCTGCTACAAGATGACACCTCA
NEK3
wildtype = NM_152720
Exon 14 skipping (135 bp)
NEK3 asv1
TACAGCTTTGGAAAATGCATCCATACTCACCTCCAGTTTAACAGCAGAGGACGATAGAGGTTCAGAAGGGTTCTT
GAAAGGCCCCCTGTCTGAAGAAACAGAAGCATCGGACAGTGTTGATGGAGGTCACGATTCTGTCATTTTGGATCC
Neu1
wildtype = NM_004210
Exon 2 and 3 skipping (564 nucleotides)
Neu1 asv1
ATGGGTAACAACTTCTCCAGTATCCCCTCGCTGCCCCGAGGAAACCCGAGCCGCGCGCCGCGGGGCCACCCCCAG
AACCTCAAAGATAGCGAGCTGGTGCTCCCGGACTGTCTGCGGCCGCGCTCCTTCACCGCCCTGCGGCGGCCGTCG
PLP1
wildype = NM_000533
Proteolipid protein 1 (Pelizaeus-Merzbacher disease, spastic paraplegia 2,
uncomplicated)
Skipping of 105 nucleotides from 5′ part of exon 3
PLP1 asv1
CCTGCTGGCTGAGGGCTTCTACACCACCGGCGCAGTCAGGCAGATCTTTGGCGACTACAAGACCACCATCTGCGG
CAAGGGCCTGAGCGCAACGTTTGTGGGCATCACCTATGCCCTGACCGTTGTGTGGCTCCTGGTGTTTGCCTGCTC
TGCTGTGCCTGTGTACATTTACTTCAACACCTGGACCACCTGCCAGTCTA
Mdm-2
wildype = Z12020
Exons 4-11 spliced out; deletion of 1020 nucleotides
mdm2 asv1
ATGTGCAATACCAACATGTCTGTACCTACTGATGGTGCTGTAACCACCTCACAGATTCCTGATTGTAAAAAAACT
ATAGTGAATGATTCCAGAGAGTCATGTGTTGAGGAAAATGATGATAAAATTACACAAGCTTCACA
VEGFR3
wildype = AY233383
Alternative usage of the last exon.
vegfr3 asv1
CATTTGAGGAATTCCCCATGACCCCAACGACCTACAAAGGCTCTGTGGACAACCAGACAGACAGTGGGATGGTGC
TGGCCTCGGAGGAGTTTGAGCAGATAGAGAGCAGGCATAGACAAGAAAGCGGCTTCAGGTAGCTGAAGCAGAGAG
AGAGAAGGCAGCATACGTCAGCATTTTCTTCTCTGCACTTATAAGAAAGATCAAAGACTTTAAGACTTTCGCTAT
TTCTTCTACTGCTATCTACTACAAACTTCAAAGAGGAACCAGGAGGACAAGAGGAGCATGAAAGTGGACAAGGAG
TGTGACCACTGAAGCACCACAGGGAGGGGTTAGGCCTCCGGATGACTGCGGGCAGGCCTGGATAATATCCAGCCT
CCCACAAGAAGCTGGTGGAGCAGAGTGTTCCCTGACTCCTCCAAGGAAAGGGAGACGCCCTTTCATGGTCTGCTG
AGTAACAGGTGCCTTCCCAGACACTGGCGTTACTGCTTGACCAAAGAGCCCTCAAGCGGCCCTTATGCCAGCGTG
ACAGAGGGCTCACCTCTTGCCTTCTAGGTCACTTCTCACAATGTCCCTTCAGCACCTGACCCTGTGCCCGCCGAT
TATTCCTTGGTAATATGAGTAATACATCAAAGAGTAGTATTAAAAGCTAATTAATCATGTTTATAAAAAAAAAAA
AAAAAAAAAAAAAAAAAAAA
pyridoxal kinase
wildype = BC000123
Alternative splice acceptor in exon 8; deletion of 87 nucleotides
pyridoxal kinase asv1
GTGGTGCCGCTTGCAGACATTATCACGCCCAACCAGTTTGAGGCCGAGTTACTGAGTGGCCGGAAGATCCACAGC
CAGGGCAGCAACTACCTGATTGTGCTGGGGAGTCAGAGGAGGAGGAATCCCGCTGGCTCCGTGGTGATGGAACGC
ATCCGGATGGACATTCGCAAAGTGGACGCC
KIAA1117
wildype = AK027030
Intron retained between exons 12 and 13; insertion of 137 nucleotides
KIAA1117 asv1
GAGCTTGGAAAAAAGAAGCTTTTGACCTCTTTATGGATCCCAGTTTCTTTCAGATGGATGCCTCTTGTGTTAATC
AGTAAGTTGCCCTCTTATTTGTATTCAGCATGATGCACCTCACAGTCTGATGAAATCAGCCACTCCCCTGGAAAG
TTAGAATACTGTTCTTTAACAGTAACAACATAATTACATGTTGTAATCCTTATCTCTTTCAGGTGGAGAGCAATT
ATGGACAATCTGATGACACATGATAAAACAACATTTAGAGATTTGATGACTCGTGTAGCAGTGGCTCAAAGCAGT
CSDA
wildype = BC021926
Alternative splice acceptor in exon 7, leads to 3 amino acid deletion;
deletion of 9 nucleotides
csda asv1
CCAACAGAATACAGGCTGGTGAGATTGGAGAGATGAAGGATGGAGTCCCAGAGGGAGCACAACTTCAGGGACCGG
TTCATCGAAATCCAACTTACCGCCCAAGCAGGGGACCTCCTCGCCCACGACCTGCCCCAGCAGTTGGAGAGGCTG
AAGATAAAGAAAATCAGCAAGCCACCAGTG
Lyk5
wildype = AK074771
2 additional exons after exon 2; insertion of 111 nucleotides
Lyk5 asv1
CAGGAACAGGTTTAAGTTTTTGAAACTGAAGTAGGTCTACACAGTAGGAACTCATGTCATTTCTTGTAAGTAAAC
CAGAGCGAATCAGGCGGTGGGTCTCGGAAAAGTTCATTGTTGAGGGCTTAAGAGATTTGGAACTATTTGGAGAGC
AGCCTCCGGGTGACACTCGGAGAAAAACCAATGATGCGAGCTCAGAGTCAATAGCATCCTTCTCTAAACAGGAGG
TCATGAGTAGCTTTCTGCCAGAGGGAGGGTGTTACGAGCTGCTCACTGTGATAGGCAAAGGATTTGAGGACCTGA
nfkb2
wildype = BC002844
Alternative exons 18, 19. Exons 18-22 spliced out; deletion of 857
nucleotides
nfkb2 asv1
GCTGCGGGCAGGCGCTGGTGCTCCTGAGCTGCTGCGTGCACTGCTTCAGAGTGGAGCTCCTGCTGTGCCCCAGCT
GTTGCATATGCCTGACTTTGAGGGACTGTATCCAGTACACCTGGCGGTCCGAGCCTCAGGTGCACTGACCTGCTG
CCTGCCCCCAGCCCCCTTCCCGGACCCCCTGTACAGCGTCCCCACCTATTTCAAATCTTATTTAACACCCCACAC
CCACCCCTCAGTTGG
FXR1
wildype = U25165
Exon 15 spliced out; deletion of 92 nucleotides
FXR1 asv1
TCACAGTACTAACCGTCGTAGGCGGTCTCGTAGACGAAGGACTGATGAAGATGCTGTTCTGATGGATGGAATGAC
TGAATCTGATACAGCTTCAGTTAATGAAAATGGGCTAGGCAAAAGATGTGATTGAAGAGCATGGTCCTTCAGAAA
AGGCAATAAACGGCCCAACTAGTGCTTCTG
M-RIP
wildype = AL834513
Exon 9 spliced out; deletion of 63 nucleotides
M-RIP asv1
GACAGTGCCACGGTGTCCGGATATGATATAATGAAATCTAAAAGCAACCCTGACTTCTTGAAGAAAGACAGATCC
TGTGTCACCCGGCAACTCAGAAACATCAGGTCCAAGAGTCTGAAGGAAGGCCTGACGGTGCAAGAACGGTTGAAG
CTCTTTGAATCCAGGGACTTGAAGAAAGAC
NPIP
wildype = BC046145
Alternative splice acceptor in exon 4; deletion of 242 nucleotides
npip asv1
ATGTTTCAACGTGCGCAAGCGTTGCGGCGGCGGGCAGAGGACTACTACAGATGCAAAATCACCCCTTCTGCAAGA
AAGCCTCTTTGCAACCGGCGGATGATAATCTCAAGACACCTCCCGAGTGTCTGCTCACTCCCCTTCCACCCTCAG
CTCTACCCTCAGCGGATGATAATCTCAAGA
HGD
wildype = AF045167
Alternative use of exons 12 and 13; deletion of 213 bp
hgd asv1
ATACACCCTACAAGTACAACCTGAAGAATTTCATGGTTATCAACTCAGTGGCCTTTGACCATGCAGACCCATCCA
TTTTCACAGTATTGACTGCTTTGAGAAGGCCAGCAAGGTCAAGCTGGCACCTGAGAGGATTGCCGATGGCACCAT
GGCATTTATGTTTGAATCATCTTTAAGTCTGGCGGTCACAAAGTGGGGACTCAAGGCCTC
TMPIT
wildype = NM_031925
Cryptic splicing, 62 bp skipped from the last exon
TMPIT asv1
AGCCATGCAGCCCCCGCCCCCGGGCCCGCTGGGCGACTGCCTGCGGGACTGGGAGGATCTACAGCAGGACTTCCA
GAACATCCAGGAGACCCATCGGCTCTACCGCCTGAAGCTGGAGGAGCTGACCAAACTTCAGAACAATTGCACCAG
CTCCATCACGCGGCAGAAGAAGCGGCTCCAGGAGCTGGCCCTCGCCCTGAAGAAATGCAAACCCTCCCTCCCAGC
AGAGGCCCAGGOGGCCGCACAGGAGCTGGAGAACCAGATGAAAGAGCGCCAAGGCCTCTTCTTTGACATGGAGGC
CTATTTGCCTAAGAAGAATGGATTGTACCTGAGCCTGGTTCTGGGGAACGTCAACGTCACGCTCCTGAGCAAGCA
GGCTAAGTTTGCCTACAAGGACGAGTATGAGAAGTTCAAGCTCTACCTCACCATCATCCTCATCCTCATCTCCTT
CACTTGCCGCTTCCTGCTCAACTCCAGGGTGACAGATGCTGCCTTCAACTTCCTGCTGGTCTGGTACTACTGCAC
CCTGACCATCCGGGAGAGCATCCTCATCAACAACGGCTCCCGGATCAAAGGCTGGTGGGTGTTCCATCACTACGT
GTCCACCTTCCTGTCGGGAGTCATGCTGACGTGGCCCGACGGTCTCATGTACCAGAAATTCCGGAACCAATTCCT
CTCCTTTTCCATGTACCAGAGCTTCGTGCAGTTTCTCCAGTACTACTACCAGAGCGGCTGCCTCTACCGCCTGCG
GGCGCTGGGCGAGCGGCACACCATGGACCTCACTGTGGAGGGCTTCCAGTCCTGGATGTGGCGGGGCCTCACCTT
CCTGCTGCCTTTTCTTTTCTTTGGACACTTCTGGCAGCTTTTTAACGCGCTGACGTTGTTCAACCTGGCCCAGGA
CCCTCAGTGCAAGGAGTGGCAGGGTTGTGCACCACAAGTTTCACAGTCAGCGGCACGGGAGCAAGAAGGATTGAG
GCTGGGCCTTCCCCTGCCGGCCCAGAGGGGCTTCTGTCCTGTGTGTTGTGGGAGGGGATGGGAGGCGCCCCTCGA
GTGTGCGTGTATCAGGGGGTCTCTTCTATTCTCCCTTGGGTTTTATGGGCGCTGTGGGCCCTGAAGGAAGACCTG
GGCCCAGTGCCCTCAATAAAGAGAG
GT335
wildype = U53003
Exon5 skipping; deletion of 93 bp
gt335 asv1
GATCGCCCGTGGCAAAATCACAGACCTGGCCAACCTCAGTGCAGCCAACCATGATGCTGCCATCTTTCCAGGAGG
CTTTGGAGCGGCTAAAAACCTCTTGTGCTGCATTGCACCTGTCCTCGCGGCCAAGGTGCTCAGAGGCGTCGAGGT
GACTGTGGGCCACGAGCAGGAGGAAGCTGGCAAGTGGCCTTATGCCGGGACCGCAGAGGC
HSSB
wildype = AF277319
Alternative splice donor in exon 1; insertion of 183 bp; splicing does not
change the protein composition
hssb asv1
CCCTGCGTGGCTGGGCTGCTCGGGTTAGATCGTCAGGTGAGGGAGGAAGGGATAGCCAGCGCGAAGGAAGTGCTG
GAGTCGTGTGTTTTGGCTGCGCGTGATCCTGCGTGGGTCGGGAGGTGTTTCTGTGTAGGTGTCTGGCCCTTTCAT
CAGTCGTGCGGAGGACCGCGTGATTTCCTTCCAGTTCTCCTCGGTTTTCAGGTGGTGGCGCCATCTTCGGAAAAG
CCTAAAGATTAGACTGTAAGAAAAGAAAATAGAAGCCATGTTTCGAAGACCTGTATTACAGGTACTTCGTCAGTT
APBB1
wildype = BC010854
Alternative splice acceptor in exon 3; insertion of 15 bp
apbb1f1 asv1
TGTTTGGCATGCGGAACAGTGCAGCCAGTGATGAGGACTCAAGCTGGGCTACCTTATCCCAGGGCAGCCCCTCCT
ATGGCTCCCCAGAGGACACAGCCTCCCACCTGGCAGATTCCTTCTGGAACCCCAACGCCTTCGAGACGGATTCCG
ACCTGCCGGCTGGATGGATGAGGGTCCAGG
OIP2
wildype = BC020773
Alternative splice acceptor in exon 6; deletion of 37 bp
oip2 asv1
AGTTGGGAAATACTACAGTAATCTGTGGAGTTAAAGCAGAATTTGCAGCACCATCAACAGATGCCCCTGATAAAG
GATACGTTGATTCCGGTCTGGACCTCCTGGAGAAGAGGCCCAAGTGGCTAGCCAATTCATTGCAGATGTCATTGA
AAATTCACAGATAATTCAGAAAGAGGACTT
UBEC2C
wildype = BC050736
Alternative 5′exon, if any protein is translated, the alternative Met is
used.
ubec2c asv1
CCAGGAGCTCAGACCGTCTTTGAGANTCTCCCGAAGGAGGAATGGGAGGGTAGGGGCGCTGCCAGACTCCTTCCC
TGGTGGGCCTAGATGAAGACGCTCAAGGACCCTCGTGACTTGGCCGAGACAGGGGAAGGGAGAAGTTGAGTCGGG
CAAGGAAGAGATGCTAAAGCCTGGGGAATTAAGAACATGCCAGAATCATCCCGAGGGAGTCTGGAATTAGGGAGG
GTGAGGACTCGCTAGGATCGTCCTGTGGATCTGGCTACAGCAGGAGCTGATGACCCTCATGATGTCTGGCGATAA
AGGGATTTCTGCCTTCCCTGAATCAGACAACCTTTTCAAATGGGTAGGGACCATCCATGG
DKFZp313H1733
wildype = BX537867
Exons 13 and 14 spliced out; deletion of 201 bp
DKFZp313H1733 asv1
ATTTCAGAGTGCCTGCCCCGGTTGACATGCATGATCAGAGGGATCGGAGACCCACTAGTGTCGGTGTATGCCCGT
GCCTACCTGTGCCGGGCTCTGCTGACCGAGATGATGGAAAGGTGTAAGAAACTAGGAAACAATGCCTTGCTGTTG
AATTCTGTGATGTCTGCCTTCCGGGCTGAG
RNF8
wildype = AB014546
Exon 7 spliced out; deletion of 205 bp
rnf8 asv1
AGCACAGAAGGAAGAAGTTCTTAGCCACATGAATGATGTGCTAGAGAATGAGCTCCAATGTATTATTTGTTCAGA
ATACTTCATTGAGCAAAGAGATTGTTCTGAAGACCGTGCTCTAAGGGCATTTGAAAGACTGCCAGGTAGTGCGAG
CCTGAGATGGTCTGGAGGATTCTCTCTAGC
PCNP
wildype = BC013916
Exons 2 and 3 spliced out; deletion of 292 bp
PCNP asv1
GGGGCTGCAGGGGAGGCCGCGGCGGGGAAAATGGCGGACGGGAAGGCGGGAGACGAGAAGCCTGAAAAGTCGCAG
CGAGCTGGAGCCGCCGGAGATACACCAACATCAGCTGGACCAAACTCCTTCAATAAAGGAAAGCATGGGTTTTCT
GATAACCAGAAGCTGTGGGAGCGAAATATA
WBP2
wildype = BC010616
Alternative splice donor site in exon1; insertion of 59 bp
wbp2 asv1
TGCGTTTTGAGTCTCGGGACCCCTGTTGGAGAGACTATGGCGCTCAACAAGAATCACTCGGAGGGCGGCGGAGTG
ATCGTCAATAACACCGAGAGGTGAAAACACTGCGGAAGGATCCTGGAGGACCAAAGTTCGGGTGTCGAGGAAGTG
GGCGCATCCTAATGTCCTATGATCACGTGGAACTCACATTCAATGACATGAAGAACGTGCCAGAAGCCTTCAAAG
GGACCAAGAAAGGCA
ALG8
wildype = BC001133
Exon 2 spliced out; deletion of 79 bp
alg8 asv1
ACAATTGCCACGGGTACTGGCAATTGGTTTTCGGCTTTGGCGCTCGGGGTGACTCTTCTCAAATGCCTTCTCATC
CCCACATAGCAACTTCAGAGTGGACGTTGGATTACCCCCCTTTCTTTGCATGGTTTGAGTATATCCTGTCACATG
TTGCCAAATATTTTGATCAAGAAATGCTGA
HNRPA2B1
wildype =
Additional exon after I exon; insertion of 36 bp, alternative initiation
codon used.
hnRNPA2B1 asv1
TCCGGTTCGTGTTCGTCCGCGGAGATCTCTCTCATCTCGCTCGGCTGCGGGAAATCGGGCTGAAGCGACTGAGTC
CGCGATGGAGAAAACTTTAGAAACTGTTCCTTTGGAGAGGAAAAAGAGAGAAAAGGAACAGTTCCGTAAGCTCTT
TATTGGTGGCTTAAGCTTTGAAACCACAGAAGAAAGTTTGAGGAACTACTACGAACAATG
ISCU2
wildype = AY009128
Additional exon after I exon; insertion of 96 bp
iscu2 asv1
AGGCGCAAGCCGGCAAGATGGCGGCGGCTGGGGCTGGCCGTCTGAGGCGGGTGGCATCGGCTCTGCTGCTGCGGA
GCCCCCGCCTGCCCGCCCGGGAGCTGTCGGCCCCGGCCCGACTCTATCACAAGAAGGTATCTCAAATCTGTGAAG
TATTGTAGAGGAGACACAAAAGGAATTGGGGGTCACAAATGGTTCTCATTGACATGAGTGTAGACCTTTCTACTC
AGGTTGTTGATCATTATGAAAATCCTAGAAACGTGGGGTCCCTTGACAAGACATCTAAAAATGTTGGAACTGGAC
TGGTGGGGGCTCCAGCATGTGGTGACGTAATGAAATTACAGATTCAAGTG
AKNAh
wildype = AB051511
3′ exon insertion after exon 1.
aknah asv1
CACAGCCTTGTAGCCGGGAGTCGCTGCCGAGTGGGCGCTCAGTTTTCGGGTCGTCATGGCTGGCTACGAATACGT
GAGCCCGGAGCAGCTGGCTGGCTTTGATAAGTACAAGCCCCCGAAAGGATGGAGTTCCTTCTGTTGTGTCAATCG
CCTTCATTTTAGTGAAGTTTCCACTCGCCTGTCATGCATACAACTTCGGAGGAGGAGATGATCGTTTGGCAGATG
AGGCCCGGGAGGGGAGCGACTTGCCGATGCCATCCTGCTGATGTCTCCACTTCTGCTCCCGGCAGGGACTTCCTA
AGCGGCAGCTTGTGGCGCTAGGGCCACCAGATGAAAGGGAGGTGCACAGGAAGGAGCTGTGGAGTGGAAAGAGCG
CGGGCTTTCGAGCACATACAAACCTGATTACAAAAGTCAGATTTCTTTAAAAAAAAAAAAAAA
A1x4
wildype = AB058691
Deletion in 3′UTR; deletion of 92 bp
A1x4 asv1
AGGAGCACAGTGCGGCCATTTCCTGGGCCACATGACAGGGCACCCCTGCCCCGTCCCCACCTCGGGACACCATGG
GCCACGCCCATGTTTTCCAGGCCCCCAGCCTCCCACTCGACTTTCCTCTTAGGAACCTGGCCCCTCCCTGGCACT
GAGGCCCTGACCCCTGCTCCCGGCCACAGGCAGTGGAGAAAGCCAGGTGGCCACGTTTTTCAGCTTCGCATCCAT
GATAAGCTGAAAGCGCTTTCTTGCTCCCGCCCACTCCTCTGCTCTGCCTAGTTGA
Tyr
wildype = M27160
Exon 3 deleted; deletion of 184 bp
Tyr asv1
GATGTAGAATTTTGCCTGAGTTTGACCCAATATGAATCTGGTTCCATGGATAAAGCTGCCAATTTCAGCTTTAGA
AATACACTGGAAGTATTTTTGAGCAGTGGCTCCGAAGGCACCGTCCTCTTCAAGAAGTTTATCCAGAAGCCAATG
ARNT
wildype = AL834279
Deletion in exon 11, exons 12-20 deleted; deletion of 1133 nucleotides
arnt asv1
AGGAACAGATGCAGGAATGGACTTGGCTCTGTAAAGGATGGGGAACCTCACTTCGTGGTGGTCCACTGCACAGGC
TACATCAAGGCCTGGCCCCCAGCAGGTGTTTCCCTCCCAGATGATGACCCAGCCTGAGGTCTTCCAGGAGATGCT
GTCCATGCTGGGAGATCAGAGCAACAGCTACAACAATGAAGAATTCCCTGATCTAACTAT
ATF3
wildype = BC006322
Additional exon before exon 4; insertion of 151 nucleotides
atf3 asv1
ATGAAAGGAAAAAGAGGCGACGAGAAAGAAATAAGATTGCAGCTGCAAAGTGCCGAAACAAGAAGAAGGAGAAGA
CGGAGTGCCTGCAGCTTCAGTATTAGCAGAGCCACAGGCCGCCTCTGTGGCATCACCAGGGTTTCTCTGAAGAAG
AGGGTCTGCATTTTCCTAAACCCAGTGCTGCTCTCCCATCTCCCATCTTCCTCTCGCAGCTTGATGAGCCCCGGT
GTGTCCCAGGAGTCGGAGAAGCTGGAAAGTGTGAATGCTGAACTGAAGGCTCAGATTGAGGAGCTCAAGAACGAG
AAGCAGCATTTGATATACATGCTCAACCTTCATCGGCCCACGTGTATTGT
BAF250
wildype = AF231056
Exon 16 deleted; deletion of 892 nucleotides
baf250 asv1
ACCCCCCGCAGCAGCAGCAGCAGCAGCAGCAACGACATGATTCCTATGGCAATCAGTTCTCCACCCAAGGCACCC
CTTCTGGCAGCCCCTTCCCCAGCCAGCAGACTACAATGTATCAACAGCAACAGCAGGAACCCCGGAGGCATGGCG
GGTAATGATGTCCCTCAAGTCTGGTCTCCTGGCAGAGAGCACATGGGCATTAGATACCATCAACATCCTGCTGTA
TGATGACAACAGCATCATGACCTTCAACCTCAGTCAGCTCCCAGGGTTGCTAGAG
BAF250
wildype = AF231056
Deletion in exon 16; deletion of 651 nucleotides
baf250 asv2
ACCCCCCGCAGCAGCAGCAGCAGCAGCAGCAACGACATGATTCCTATGGCAATCAGTTCTCCACCCAAGGCACCC
CTTCTGGCAGCCCCTTCCCCAGCCAGCAGACTACAATGTATCAACAGCAACAGCAGGTATCCAGCCCTGCTCCCC
TGCCCCGGCCAATGGAGAACCGCACCTCTCCTAGCAAGTCTCCATTCCTGCACTCTGGGATGAAAATGCAGAAGG
CAGGTCCCCCAGTACCTGCCTCGCACATAGCACCTGCCCCTGTGCAGCCCCCCAT
BRF1
wildype = AJ297407
Exons 5-11 deleted, deletion in exon 12; deletion of 2044 nucleotides
brf1 asv1
GAGGCTCACGGAATTTGAAGACACCCCCACCAGTCAGTTGACCATTGATGAGTTCATGAAGATCGACCTGGAGGA
GGAGTGCGACCCCCCCATCGAGGAGGGAGGGCAGACGGAGGCCCGAGAGCCTCCCCAGGCCTCTTCGTGGGAAGG
CCCCAGTACCACTCGTAGGAGGTCTCAGCTCTGGCATGGCTGCCCCGGATGTGGCCGAGG
BRF1
wildype = AJ297407
Different 5′ region
brf1 asv2
CGGCCGCGTCGACCGGCTGCGCTCACCGGTAGGCCCCGCTCGGGTTCCGCCGAAGCCCAGCCCCCGCAGGTCGGC
CCCTCCGACGCCGGCCGCGCCGCAAGGGAGGCCAGCTCGCTCGCAGTGGGGAGGTCGCGGCTCCAGTCCTCGCGT
CCCCGCCGTGGTCCCGGTGCCTGTCCCATCCCGCGGGCGGGGCCGTTGCGGGGCCGGGCCCGGGCCGGGGCGAAT
CTGCGGCTGCGAATCGGCTGGAGCGGGGCCTCGCGAGAGGCCGAGGCTGGGCGGCTGGGCTGGGCGGGCGGCCGG
GGCTGCTCCGGAGGCTCGGGTGGCTTGAGAGTCTTGGGAGGCTCCGCCTGCCCGCCGGTCGCCGGCATGACGGGC
CGCGTGTGCCGCGGTTGCGGCGGCACGGACATCGAGCTGGACGCGGCCCGCGGGGACGCGGTGTGCACCGCCTGC
GGCTCAGTGCTGGAGGACAACATCATCGTGTCCGAGGTGCAGTTCGTGGAGAGCAGCGGCGGCGGCTCCTCGGCC
GTGGGCCAGTTCGTGTCCCTGGACGGTGCTGGCAAAACCCCGACTCTGGGTGGCGGCTTCCACGTGAATCTGGGG
AAGGAGTCGAGAGCGCAGACCCTGCAGGATGGGAGGCGCCACATCCACCACCTGGGGAACCAGCTGCAGCTGAAC
CAGCACTGCCTGGACACCGCCTTCAACTTCTTCAAGATGGCCGTGAGCAGGCACCTGACCCGCGGCCGGAAGATG
GCCCACGTGATTGCTGCCTGCCTCTACCTGGTCTGCCGTACGGAGGGCACGCCGCACATGCTCCTGGTCCTCAGC
GACCTGCTCCAGGTGAATGTGTACGTGCTTGGAAAGACGTTTCTTCTCTTGGCAAGAGAGCTCTGCATCAATGCG
CCGGCCATAGACCCGTGCCTGTATATTCCACGCTTTGCGCACCTGCTGGAATTCGGGGAGAAGAACCACGAGGTG
TCCAT
ELF3
wildype = AF017307
Insertion in 5′ UTR; insertion of 114 nucleotides
elf3 asv1
CTCCGCCACTCCGGTAGGATTCCCCGCCTGTCATTCCCTAGCCCAGCTCTTGGGAAACTGCAGAGGGGTCCAGAG
GATTTGCAGTTCTGAACCTGCACACTCCAGTCTAGGATCTCCGAGCAAGAGCGTAGCCTCATGGCTACAACCTGT
GAGATTAGCAACATTTTTAGCAACTACTTCAGTGCGATGTACAGCTCGGAGGACTCCACC
ELF3
wildype = AF017307
Deletion in exon 5; deletion of 69 nucleotides
elf3 asv2
GCTGCGAGACCTCACTTCCAGCTCTTCTGATGAGCTCAGTTGGATCATTGAGCTGCTGGAGAAGGATGGCATGGC
CTTCCAGGAGGCCCTAGACCCAGGGCCCTTTGACCAGGGCAGCCCCTTTGCCCAGGAGCTGCTGGACGACGTCTC
CACCGCAGGGACTGGTGCTTCTCGGAGCTCCCACTCCTCAGACTCCGGTGGAAGTGACGTGG
Hes6
wildype = BC007939
Deletion in exon 3; deletion of 6 nucleotides
hes6 asv1
CCGCAAGGCCCGGAAGCCCCTGGTGGAGAAGAAGCGGCGCGCGCGGATCAACGAGAGCCTGCAGGAGCTGCGGCT
GCTGCTGGCGGGCGCCGAGGCCAAGCTGGAGAACGCCGAAGTGCTGGAGCTGACGGTGCGGCGGGTCCAGGGTGT
GCTGCGGGGCCGGGCGCGCGAGCGCGAGCAGCTGCAGGCGGAAGCGAGCGAGCGCTTCGC
Hes6
wildype = BC007939
Intron retained between exons 3 and 4; insertion of 235 nucleotides
hes6 asv2
CTGCTGCTGGCGGGCGCCGAGGTGCAGGCCAAGCTGGAGAACGCCGAAGTGCTGGAGCTGACGGTGCGGCGGGTC
CAGGGTGTGCTGCGGGGCCGGGCGCGCGGTGAGTGGCGGCGGGGCGGGCGGGGGCGCCGGCCGCGGGCGCCTGTA
ACCCCTGCCAGACGGAGGACTTCCCTCCCGGCGCCCCTGTCCTGTCGGCGGCGAGGGCTCCCACCGGAGCAGGGT
GCGCCCCCGCGTCTCCTGGGTGAGCCGCGTCCCCGCGGGCCGGGTGGGCTGGGCCACGCAGTCGCCGCTCACCGC
GCGGGACGCGGCTCTCTCCCTCCCACCCTCGGGCCCAGAGCGCGAGCAGCTGCAGGCGGAAGCGAGCGAGCGCTT
CGCTGCCGGCTACATCCAGTGCATGCACGAGGTGCACACGTTCGT
HesR1
wildype = BC001873
Exon 3 longer, deletion in 3′UTR; insertion of 12 nucleotides; deletion of
nucleotides in 3′ UTR.
hesr1 asv1
GAAGCGCCGACGAGACCGGATCAATAACAGTTTGTCTGAGCTGAGAAGGCTGGTACCCAGTGCTTTTGAGAAGCA
GGTAATGGAGCAAGGATCTGCTAAGCTAGAAAAAGCCGAGATCCTGCAGATGACCGTGGATCACCTGAAAATGCT
GCATACGGCAGGAGGGAAAGGTTACTTTGACGCGCACGCCCTTGCTATGGACTATCGGAG
HOXA1
wildype = S79869
Two deletions in exon 1; deletion of 203 nucleotides and deletion of 466
nucleotides; deletion of 669 nucleotides in total
hoxa1 asv1
CACCACCCCCAGCCGGCTACCTACCAGACTTCCGGGAACCTGGGGGTGTCCTACTCCCACTCAAGTTGTGGTCCA
AGCTATGGCTCACAGAACTTCAGTGCGCCTTACAGCCCCTACGCGTTAAATCAGGAAGCAGACCCACCAAGAAGC
CTGTCGCTCCCCCGCATCGGAGACATCTTCTCCAGCGCAGACTTTTGACTGGATGAAAGTCAAAAGAAACCCTCC
CAAAACAGGGAAAGTTGGAGAGTACGGCTACCTGGGTCAACCCAACGCGGTGCGCACCAACTTCACTACCAAGCA
GCTCACGGAACTGGAGAAGGAGTTCCACTTCAACAAGTACCTGACGCGCG
HOXA1
wildype = S79869
One deletion in exon 1; deletion of 466 nucleotides
hoxa1 asv2
AGCCTGTCGCTCCCCCGCATCGGAGACATCTTCTCCAGCGCAGACTTTTGACTGGATGAAAGTCAAAAGAAACCC
TCCCAAAACAGGGAAAGTTGGAGAGTACGGCTACCTGGGTCAACCCAACGCGGTGCGCACCAACTTCACTACCAA
GCAGCTCACGGAACTGGAGAAGGAGTTCCACTTCAACAAGTACCTGACGCGCGCCCGCAG
HRY
wildype = AK000415
Deletion in exon 1; deletion of 9 nucleotides
hry asv1
CGTGAAGAACTCCAAAAATAAAATTCTCTAGAGATAAAAAAAAAAAAAAAAGGAAAATGCCAGCTGATATAATGG
AGAAAAATTCCTCGTCCCCGGTAGCAGCCAGTGTCAACACGACACCGGATAAACCAAAGACAGCATCTGAGCACA
GAAAGTCATCAAAGCCTATTATGGAGAAAAGACGAAGAGCAAGAATAAATGAAAGTCTGA
AP-4
wildype = BC012925
Deletion in exon 14; deletion of 57 nucleotides
ap-4 asv1
ACATCTCCGCGGAGCAGAAGCGGCGCTTCAACATCAAGCTGGGGTTTGACACCCTTCATGGGCTCGTGAGCACAC
TCAGTGCCCAGCCCAGCCTCAAGGAGCGTGCGGGCTTGCAGGAGGAGGCCCAGCAGCTGCGGGATGAGATTGAGG
AGCTCAATGCCGCCATTAACCTGTGCCAGCAGCAGCTGCCCGCCACAGGGGTACCCATCA
MOX1
wildype = U10492
Exon 2 deleted; deletion of 173 nucleotides
mox1 asv1
GGCCCGGCAGGGGGTTCCAAGGAAATGGGGACCAGCAGCCTGGGCCTGGTGGACACCACAGGAGGCCCAGGCGAT
GACTACGGGGTGCTTGGGAGCACTGCCAATGAGACAGAGAAGAAATCATCCAGGCGGAGAAAGGAGAGTTCAGGT
CAAAGTGTGGTTCCAGAACCGAAGGATGAAGTGGAAGCGTGTGAAGGGAGGTCAGCCCATCTCCCCCAATGGGCA
GGACCCTGAGGATGGGGACTCCACAGCCTCTCCAAGTTCAGAGTGAGATTCTGCA
RPGR
wildype = BC031624
Additional exon between exons 15 and 16; insertion of 39 nucleotides
rpgr asv1
TGTGAAGGTGCATGGAGGAAGAAAGGAGAAAACAGAGATCCTATCAGATGACCTTACAGACAAAGCAGAGTATTC
TGCCAGTCACTCCCAAATTGTTTCAGTTTAAAAGGATCATGAATTTTCTAAAACTGAGGAACTAAAACTAGAAGA
TGTGGATGAGGAAATTAATGCTGAAAATGTGGAAAGCAAGAAGAAAACTGTGGGAGATGA
TNNT2
wildype = X74819
Exons 3, 4 and 12 deleted; deletion of 22 nucleotides and deletion of 9
nucleotides; deletion of 31 nucleotides in total
tnnt2 asv1
GAGCAGACGCCTCCAGGATCTGTCGGCAGCTGCTGTTCTGAGGGAGAGCAGAGACCATGTCTGACATAGAAGAGG
TGGTGGAAGAGTACGAGGAGGAGTGAAGCAGGAGGAGGCAGCGGAAGAGGATGCTGAAGCAGAGGCTGAGACCGA
GGAGACCAGGGCAGAAGAAGATGAAGAAGAAGAGGAAGCAAAGGAGGCTGAAGATGGCCCAATGGAGGAGTCCAA
ACCAAAGCCCAGGTCGTTCATGCCCAACTTGGTGCCTCCCAAGATCCCCGATGGAGAGAGAGTGGACTTTGATGA
CATCCACCGGAAGCGCATGGAGAAGGACCTGAATGAGTTGCAGGCGCTGATCGAGGCTCACTTTGAGAACAGGAA
GAAAGAGGAGGAGGAGCTCGTTTCTCTCAAAGACAGGATCGAGAGACGTCGGGCAGAGCGGGCCGAGCAGCAGCG
CATCCGGAATGAGCGGGAGAAGGAGCGGCAGAACCGCCTGGCTGAAGAGAGGGCTCGACGAGAGGAGGAGGAGAA
CAGGAGGAAGGCTGAGGATGAGGCCCGGAAGAAGAAGGCTTTGTCCAACATGATGCATTTTGGGGGTTACATCCA
GAAGACAGAGCGGAAAAGTGGGAAGAGGCA
GACTGAGCGGGAAAAGAAGAAGAAGATTCTGGCTGAGAGGAGGAAGGTGCTGGCCATTGACCACCTGAAT
WT1
wildype = X51630
Deletion in exon 9; deletion of 9 nucleotides
wt1 asv1
GAAACCATTCCAGTGTAAAACTTGTCAGCGAAAGTTCTCCCGGTCCGACCACCTGAAGACCCACACCAGGACTCA
TACAGGTGAAAAGCCCTTCAGCTGTCGGTGGCCAAGTTGTCAGAAAAAGTTTGCCCGGTCAGATGAATTAGTCCG
CCATCACAACATGCATCAGAGAAACATGACCAAACTCCAGCTGGCGCTTTGAGGGGTCTC
WT1
wildype = X51630
Exon 5 deleted; deletion of 51 nucleotides
wt1 asv2
CTGAGGACGCCCTACAGCAGTGACAATTTATACCAAATGACATCCCAGCTTGAATGCATGACCTGGAATCAGATG
AACTTAGGAGCCACCTTAAAGGGCCACAGCACAGGGTACGAGAGCGATAACCACACAACGCCCATCCTCTGCGGA
GCCCAATACAGAATACACACGCACGGTGTCTTCAGAGGCATTCAGGATGTGCGACGTGTG
MITF
wildype = AB006909
Different 5′ region, 3′ exon inserted after exon 3
mitf asv1
CTTTGCCAGTCCATCTTCAAATTGGAATTATAGAAAGTAGAGGGAGGGATAGTCTACCGTCTCTCACTGGATTGG
TGCCACCTAAAACATTGTTATGCTGGAAATGCTAGAATATAATCACTATCAGGTGCAGACCCACCTCGAAAACCC
CACCAAGTACCACATACAGCAAGCCCAACGGCAGCAGGTAAAGCAGTACCTTTCTACCACTTTAGCAAATAAACA
TGCCAACCAAGTCCTGAGCTTGCCATGTCCAAACCAGCCTGGCGATCATGTCATGCCACCGGTGCCGGGGAGCAG
CGCACCCAACAGCCCCATGGCTATGCTTACGCTTAACTCCAACTGTGAAAAAGAGTTTATGAAGCAGTGAGAATG
CAGAGAGAGGAGAAGGGGAGGTGGAAAAGGAAAAGCAAAAATAGAAGAGGTGTGGGACATGCTGTTTAGAAGTTC
CGCTTGTTGTGAATGTCTGGAATATTATTTTTATTTCTCCCTGAGTTGGGGGAAGAAAGAATGGAATATGCATGG
ATGGATTTGAATCATATAGCACATGAGACTTTAACGGAAACGCAAAGGTTTAATTGCTGGATACATTCTGTTTCA
TAATAAAATTGCCACTGCCCGTTAAATCTGCTTTGGTGAAGGCTGGATTGGAAACAAGACTCAAACTACCTTCAA
GCTAATTGGTGCATCAAAATTTGCAGCATACAAATACCTGAGAGCTGTGATTTAATGCTCATTATTTCCAAATTA
TGAGATGATGAGCTTCATCTCAATGGGATTTACCGTACTATGGACTATGAAGTGTTTATGCAAATTCGGAGGCAA
CTTTTCTAGAGTTGGATTGATTTTAATTTCTAGAGGGACTAAAATCTTTGCCCCTATGCCCAAACCAACTGCTTT
ATTTTTCTCTACCCAAATTTGTCATCTAGCAAGATGATTTGACACAAGTTCTTCCTTCATTATTTCATCTTTTGG
TCAGATTCCACTTTGTTTGAAAGCTTAGTTCATCTTGTTGCTGTGCCATCAGCTTTGTGTGAACAGGTCATTAAA
AAGTCATTTGCAAATCCAAAAAAAAAAAAAAA
NYBR1
wildype = AF269088
Exon 17 deleted, 6 additional alternative exons after exon 2.2; deletion of
29 nucleotides (exon 17).
NYBR1 asv1
AGAGTCCCTGTGAGACGGTTTCACAGAAGGATGTGTATTTACCCAAAGCTACACATCAAAAAGAATTCGATACCT
TAAGTGGAAAATTAGAAGCCTACCTGTGGAAGGAAAGTTTCTCTTCCAAATAAAGCCTTAGAATTAAAGGACAGA
GAAACATTCAAAGCAGAGTCTCCTGATAAAGATGGTCTTCTGAAGCCTACCTGTGGAAGGAAAGTTTCTCTTCCA
AATAAAGCCTTAGAATTAAAGGACAGAGAAACACTCAAAGCAGAGTCTCCTGATAATGATGGTCTTCTGAAGCCT
ACCTGTGGAAGGAAAGTTTCTCTTCCAAATAAAGCTTTAGAATTGAAGGACAGAGAAACATTCAAAGCAGCTCAG
ATGTTCCCATCAGAATCCAAACAAAAGGATGATGAAGAAAATTCTTGGGATTTTGAGAGTTTCCTTGAGGCTCTC
TTACAGAATGATGGGTGTTTACCCAAGGCTACACATCAAAAAGAATTCGATACCTTAAGTGGAAAATTAGAAGAG
TCTCCTGATAAAGATGGTCTTCTGAAGCCTACCTGTGGAAGGAAAGTTTCTCTTCCAAATAAAGCCTTAGAATTA
AAGGACAGAGAAACACTCAAAGCAGAGTCTCCTGATAAAGATGGTCTTCTGAAGCCTACCTGTGTAAGGAAAGTT
TCTCTTCCAAATAAAGCCTTAGAATTAAAGGACAGAGAAACATTAAAAGCAGCTCAGATGTTCCCATCAGAATCC
AAACAAAAGGATGATGAAGAAAATTCTTGGGATTTTGAGAGTTTCCTTGAGACTCTCTTACAGAATGATGTGTGT
TTACCCAAGGCTACACATCAAAAAGAATTCGATACCTTAAGTGGAAAATTAGAAGATTTCAGGCCGGGCACTGTG
GTTCACGCCTGTAATCCCAGCCCTTTGGGAGGCAGAGGCATGCGGATCACGAGGTCAGCAGATCGAGACCATCCT
GGCTAACATGGTGAAACCCCGTCTCTATGAAAAAATACAAAAAATTAGCCAAGCATGGTGGTGGGTGCCTCTAGT
CCCAGCTACTCGGGAGGCTGAGGCAGGAGAATGTGAGAACCCATGAGGCAGAGATTGCAGTGAGCCAAGATCATG
CACCTACACTCCAGCCTGGGTGACAGGGCCAGACTCTGTGAAAAAAAAAAAAAAAAAAGAATTTATTTATTGTGG
CACTATTCACAACAGCAAAGACTTGGAACCAAACCAAATGTCCAACAACGCTAGACTGGATTAAGAAAGTATGGC
ACATATACACCATGGAACACTACGCAGCCATAAAAAATGATAAGTTCATGTCCTTTGTAGGGACATGAATGAAAC
TGGAAACCATCATTCTCAGCAAACTCTCGCAAGGACAAAAAACCAAACACTGCGTGTTCTCACTCATAGGTGTGA
ATTGAACAATGAGAACACATGGACACAGGAAGGGGAACATCACACTCCGGGGACTGTTGTGGGGTTGGAGGAGGG
ATAGCATTAGGAGATATACCTAATGCTAAATGACGAGTTAATGGGAACCTGCACATTGTGCACATGTACCCTAAA
ACTTAAAGTATAATATTAAAATAAAAAATAAAGAAAAAAAAAAAAAAA
Oct1
wildype = BC052274
Alternative exon 2 used, additional exon after exon 3; insertion of 289
nucleotides (additional exon after exon 3).
oct1 asv1
AAAAATGGCGGACGGAGGAGCAGCGAGTCAAGATGAGAGTTCAGCCGCGGCGGCAGCAGCAGCAGCTACTACTGG
GCTGTAAACAGTGATGCCAGCAAAATGTTACTTCAGCTGATGAAGTGATGCTGTTTCGAGAATTTGAAAGCAATT
TTTCAGTGGATAAAGAAGTTGACAGCACGATTTGTTGGATGTGATGAAGGATTAATCAGCATACACCTTCACTTG
TATTAGCTTAAGATGGAATGGTTCTGGGCAATATAAAATAACAGACTCAAGAATGAACAATCCGTCAGAAACCAG
TAAACCATCTATGGAGAGTGGAGATGGCAACACAGCATGGACCCTTTTATGATATGGGCACTGAAACTAAAGCAC
ATGGTGGAAGAAGGATTGGTAGCATATAGAAACATTTTTAGACAAATGAAAAAGCAAAAAAGTCAGAAATTACAG
TGTATTTCCATAAAGTTACACCAAGTGTGCCTGCCTCTCCTGCCTCCCCTTCCAGCTTTTTGTCTTCTGCCATTT
CTGAGTCAGCAAGACCCCTCCTGTTCCTCCTTCTCAGCCTACTCAGCATGAAGACAAGGATGAAGATCTTTGTGA
TGATCCACTTCCACTTAATGAATAGCACACAAACCAATGGTCTGGACTTTCAGAAGCAGCCTGTGCCTGTAGGAG
GAGCAATCTCAACAGCCCAGGCGCA
Oct1
wildype = BC052274
OCTAMER-BINDING TRANSCRIPTION FACTOR 1
Exon 2 deleted; deletion of 101 nucleotides in 5′ UTR
oct1 asv2
GAGGAGCAGCGAGTCAAGATGAGAGTTCAGCCGCGGCGGCAGCAGCAGCAGACTCAAGAATGAACAATCCGTCAG
AAACCAGTAAACCATCTATGGAGAGTGGAGATGGCAACACAGGCACACAAACCAATGGTCTGGAC
Oct2
wildype = X13810
Deletion in exon 13; deletion of 136 nucleotides
oct2 asv1
GCTACAGCCCCCATATGGTCACACCCCAAGGGGGCGCGGGGACCTTACCGTTGTCCCAAGCTTCCAGCAGTCTGA
GCACAACAGCACAAACCCCAGCCCTCAAGGCAGCCACTCGGCTATCGGCTTGTCAGGCCTGAACCCCAGCACGGG
CCCTGGCCTCTGGTGGAACCCTGCCCCTTACCAGCCTTGATGGCAGCGGGAATCTGGTGC
PAX2
wildype = L25597
Additional exon inserted after exon 5, exon 9 deleted; insertion of 69
nucleotides (additional exon); deletion of 83 nucleotides (exon 9)
pax2 asv1
ACGGCCTCCCCTCCTGTTTCCAGCGCCTCCAATGACCCAGTGGGATCCTACTCCATCAATGGGATCCTGGGGATT
CCTCGCTCCAATGGTGAGAAGAGGAAACGTGATGAAGTTGAGGTATACACTGATCCTGCCCACATTAGAGGAGGT
GGAGGTTTGCATCTGGTCTGGACTTTAAGAGATGTGTCTGAGGGCTCAGTCCCCAATGGAGATTCCCAGAGTGGT
GTGGACAGTTTGCGGAAGCACTTGCGAGCTGACACCTTCACCCAGCAGCAGCTGGAAGCTTTGGATCGGGTCTTT
GAGCGTCCTTCCTACCCTGACGTCTTCCAGGCATCAGAGCACATCAAATCAGAACAGGGGAACGAGTACTCCCTC
CCAGCCCTGACCCCTGGGCTTGATGAAGTCAAGTCGAGTCTATCTGCATCCACCAACCCTGAGCTGGGCAGCAAC
GTGTCAGGCACACAGACATACCCAGTTGTGACTGGTCGTGACATGGCGAGCACCACTCTGCCTGGTTACCCCCCT
CACGTGCCCCCCACTGGCCAGGGAAGCTACCCCACCTCCACCCTGGCAGGAATGGTGCCTGGGAGCGAGTTCTCC
GGCAACCCGTACAGCCACCCCCAGTACACGGCCTACAACGAGGCTTGGAGATTCAGCAACCCCGCCTTACTAAGT
TCCCCTTATTATTATAGTGCCGCCC
CD151
wildype = NM_139030
Additional exon after exon 2. Ins 60 nucleotides. Splicing does not change
the protein.
cd151 asv1
CGCCCCCGCAGCTGCCGCCGCCGCCAGGGCCCGGACTCGGACGCGTGGTAGCCTAGAGTCCTGGGGAGCTTCTGT
CCACCTGTCCTGCAGAGGAGTCGTTTCCAGCCCGGGCCCCAGGATGGGTGAGTTCAACGAGAAGAAGACAACATG
TGGCACCGTTTGCCTCAAGTACCTGCTGTTTACCTACAATTGCTGCTTCTGGCTGGCTGGCCTGGCTGTCATGGC
PCF
wildype = X92720
Alternative splice acceptor inside exon 10
pcf asv1
CCCGCCTTCCATACCTCCCCGGCTCCGCTCGGTTCCTGGCCACCCCGCAGCCCCTGCCCAGGTGCCATGGCCGCA
TTGTACCGCCCTGGCCTGCGGCTTAACTGGCATGGGCTGAGCCCCTTGGGCTGGCCATCATGCCGTAGCATCCAG
ACCCTGCGAGTGCTTAGTGGAGATCTGGGCCAGCTTCCCACTGGCATTCGAGATTTTGTAGAGCACAGTGCCCGC
CTGTGCCAACCAGAGGGCATCCACATCTGTGATGGAACTGAGGCTGAGAATACTGCCACACTGACCCTGCTGGAG
CAGCAGGGCCTCATCCGAAAGCTCCCCAAGTACAATAACTGCTGGCTGGCCCGCACAGACCCCAAGGATGTGGCA
CGAGTAGAGAGCAAGACGGTGATTGTAACTCCTTCTCAGCGGGACACGGTACCACTCCCGCCTGGTGGGGCCTGT
GGGCAGCTGGGCAACTGGATGTCCCCAGCTGATTTCCAGCGAGCTGTGGATGAGAGGTTTCCAGGCTGCATGCAG
GGCCGCACCATGTATGTGCTTCCATTCAGCATGGGTCCTGTGGGCTCCCCGCTGTCCCGCATCGGGGTGCAGCTC
ACTGACTCAGCCTATGTGGTGGCAAGCATGCGTATTATGACCCGACTGGGGACACCTGTGCTTCAGGCCCTGGGA
GATGGTGACTTTGTCAAGTGTCTGCACTCCGTGGGCCAGCCCCTGACAGGACAAGGGGAGCCAGTGAGCCAGTGG
CCGTGCAACCCAGAGAAAACCCTGATTGGCCACGTGCCCGACCAGCGGGAGATCATCTCCTTCGGCAGCGGCTAT
GGTGGCAACTCCCTGCTGGGCAAGAAGTGCTTTGCCCTACGCATCGCCTCTCGGCTGGCCCGGGATGAGGGCTGG
CTGGCAGAGCACATGCTGATCCTGGGCATCACCAGCCCTGCAGGGAAGAAGGCGCTATGTGCAGCCGCCTTCCCT
AGTGCCTGTGGCAAGACCAACCTGGCTATGATGCGGCCTGCACTGCCAGGCTGGAAAGTGGAGTGTGTGGGGGAT
GATATTGCTTGGATGAGGTTTGACAGTGAAGGTCGACTCCGGGCCATCAACCCTGAGAACGGCTTCTTTGGGGTT
GCCCCTGGTACCTCTGCCACCACCAATCCCAACGCCATGGCTACAATCCAGAGTAACACTATTTTTACCAATGTG
GCTGAGACCAGTGATGGTGGCGTGTACTGGGAGGGCATTGACCAGCCTCTTCCACCTGGTGTTACTGTGACCTCC
TGGCTGGGCAAACCCTGGAAACCTGGTGACAAGGAGCCCTGTGCACATCCCAACTCTCGATTTTGTGCCCCGGCT
CGCCAGTGCCCCATCATGGACCCAGCCTGGGAGGCCCCAGAGGGTGTCCCCATTGACGCCATCATCTTTGGTGGC
CGCAGACCCAAAGGGGTACCCCTGGTATACGAGGCCTTCAACTGGCGTCATGGGGTGTTTGTGGGCAGAGCCATG
CGCTCTGAGTCCACTGCTGCAGCAGAACACAAAAGGACTTCTGGGAACAGGAGGTTCGTGACATTCGGAGCTACC
TGACAGAGCAGGTCAACCAGGATCTGCCCAAAGAGGTGTTGGCTGAGCTTGAGGCCCTGGAGAGACGTGTGCACA
AAATGTGACCTGAGGCCTAGTCTAGCAAGAGGACATAGCACCCTCATCTGGGAATAGGGAAGGCACCTTGCAGAA
AATATGAGCAATTGATATTAACTAACATCTTCAATGTGCCATAGACCTTCCCACAAAGACTGTCCAATAATAAGA
GATGCTTATCTATTTTAAAAAAAAAAAAAAAAAA
ZNF398
wildype = AY049743
Different 5′ region
znf398 asv1
TTAGACAGCGCAGGGCCATGGCTGAGGCGGCCCCGGCCCCGACATCTGAATGGGACTCCGAGTGCCTTACATCCC
TGCAGCCCCTTCCTCTTCCTACACCCCCAGCAGCAAATGAGGCACACCTGCAGACAGCAGCTATC
BIN1
wildype = U87558
Exons 12 and 13 deleted; deletion of 261 nucleotides
bin1 asv1
CTGCCGCCACCCCCGAGATCAGAGTCAACCACGAGCCAGAGCCGGCCGGCGGGGCCACGCCCGGGGCCACCCTCC
CCAAGTCCCCATCTCAGCCCACAGAGAGTCCAGCCGGCAGCCTGCCTTCCGGGGAGCCCAGCGCTGCCGAGGGCA
CCTTTGCTGTGTCCTGGCCCAGCCAGACGGCCGAGCCGGGGCCTGCCCAACCAGCAGAGG
BIN1
wildype = U87558
Exon 12 deleted; deletion of 129 nucleotides
bin1 asv2
CTGCCGCCACCCCCGAGATCAGAGTCAACCACGAGCCAGAGCCGGCCGGCGGGGCCACGCCCGGGGCCACCCTCC
CCAAGTCCCCATCTCAGTTTGAGGCCCCGGGGCCTTTCTCGGAGCAGGCCAGTCTGCTGGACCTGGACTTTGACC
CCCTCCCGCCCGTGACGAGCCCTGTGAAGGCACCCACGCCCTCTGGTCAGTCAATTCCAT
EAAT2
wildype = D85884
Exon 8 deleted; deletion of 135 nucleotides.
eaat2 asv1
CGCCATCTTTATAGCCCAAATGAATGGTGTTGTCCTGGATGGAGGACAGATTGTGACTGTAAGGGACAGGATGAG
AACTTCAGTCAATGTTGTGGGTGACTCTTTTGGGGCTGGGATAGTCTATCACCTCTCCAAGTCTGAGCTGGATAC
EAAT2
wildype = D85884
Exon 6 deleted; deletion of 234 nucleotides
eaat2 asv2
GATGGGAGATCAGGCCAAGCTGATGGTGGATTTCTTCAACATTTTGAATGAGATTGTAATGAAGTTAGTGATCAT
GATCATGTGTGCTGGAACTTTGCCTGTCACCTTTCGTTGCCTGGAAGAAAATCTGGGGATTGATAAGCGTGTGAC
EAAT2
wildype = D85884
Deletion in exon 5, exon 6 deleted; deletion of 334 nucleotides
eaat2 asv3
AGACTAAGATGGTTATCAAGAAGGGCCTGGAGTTCAAGGATGGGATGAACGTCTTAGGTCTGATAGGGTTTTTCA
TTGCTTTTGTGCTGGAACTTTGCCTGTCACCTTTCGTTGCCTGGAAGAAAATCTGGGGATTGATAAGCGTGTGAC
ELF1
wildype = M82882
Retained intron; insertion of 118 nucleotides
elf1/1 asv1
GAAGAGCCCAATGACATGATTACTGAGAGTTCACTGGATGTTGCTGAAGAAGAAATCATAGACGATGATGATGAT
GACATCACCCTTACAGTGGAAACAGGGTTTCTCCATGTTGGCCAGTCTCAGACTCCTGACCTCAAGCAATCTGCT
TGCCTCGGCTTCCCAAAGTGCGGGATTACAGGAATGAGCCACTGCGCCAGCCAGGTTTGTTGAAGCTTCTTGTCA
TGACGGGGATGAAACAATTGAAACTATTGAGGCTGCTGAGGCACTCCTCAATATG
ELF1
wildype = M82882
Additional 5′ exon, deletion in exon 1, exons 2-4 deleted; deletion of 797
nucleotides
elf1 asv2
GAGCAGCGGCGGCGGCGGCGGCGGCGGCAGCAGCAGCTTCAGTAGCGCAGAGGCGGCGGTGGCGAGAGGTGCGGC
GAAGGAGGCAGAGGCACTTATGCTTGTCAGGCCAAGAAGCTTGAGAGAAGAAAAATTTCAGAAAAATTGTCTCAA
TTTGACTAGAATATCAATGAACCAGGAAAAAAGGAAGAAAAACTAAACCACCATGACCAGATTCCCCAGCCACTA
CGCCAAATATATCTGTGAAGAAGAAAAACAAAGATGGAAAGGGAAACACAATTTA
FGFR2
wildype = M87770
Exons 2 and 3 deleted, alternative exon 5; deletion of 345 nucleotides
(exons 2 and 3)
fgfr2 asv1
GGATTGGTACCGTAACCATGGTCAGCTGGGGTCGTTTCATCTGCCTGGTCGTGGTCACCATGGCAACCTTGTCCC
TGGCCCGGCCCTCCTTCAGTTTAGTTGAGGATACCACATTAGAGCCAGAAGGAGCACCATACTGGACCAACACAG
AAAAGATGGAAAAGCGGCTCCATGCTGTGCCTGCGGCCAACACTGTCAAGTTTCGCTGCCCAGCCGGGGGGAACC
CAATGCCAACCATGCGGTGGCTGAAAAACGGGAAGGAGTTTAAGCAGGAGCATCGCATTGGAGGCTACAAGGTAC
GAAACCAGCACTGGAGCCTCATTATGGAAAGTGTGGTCCCATCTGACAAGGGAAATTATACCTGTGTGGTGGAGA
ATGAATACGGGTCCATCAATCACACGTACCACCTGGATGTTGTGGAGCGATCGCCTCACCGGCCCATCCTCCAAG
CCGGACTGCCGGCAAATGCCTCCACAGTGGTCGGAGGAGACGTAGAGTTTGTCTGCAAGGTTTACAGTGATGCCC
AGCCCCACATCCAGTGGATCAAGCACGTGGAAAAGAACGGCAGTAAATACGGGCCCGACGGGCTGCCCTACCTCA
AGGTTCTCAAGGCCGCCGGTGTTAACACCACGGACAAAGAGATTGAGGTTCTCTATATTCGGAATGTAACTTTTG
AGGACGCTGGGGAATATACGTGCTTGGCGGGTAATTCTATTGGGATATCCTTTCACTCTGCATGGTTGACAGTTC
TGCCAGCGCCTGGAAGAGAAAAGGAGATTACAGCTTCCCCAGACTACCTGGAGATAGCCATTTACTGCATAGGGG
TCTTCTTAATCGCCT
GABARG2
wildype = BC059389
Exon 9 deleted; deletion of 24 nucleotides
gabarg2 asv1
TGTCTTCTCTGCTCTGGTGGAGTATGGCACCTTGCATTATTTTGTCAGCAACCGGAAACCAAGCAAGGACAAAGA
TAAAAAGAAGAAAAACCCTGCCCCTACCATTGATATCCGCCCAAGATCAGCAACCATTCAAATGAATAATGCTAC
ACACCTTCAAGAGAGAGATGAAGAGTACGGCTATGAGTGTCTGGACGGCAAGGACTGTGC
GATA1
wildype = X17254
Deletion in exon 6; deletion of 335 nucleotides
gata1 asv1
TGTCAGTAAACGGGCAGGTACTCAGTGCACCAACTGCCAGACGACCACCACGACACTGTGGCGGAGAAATGCCAG
TGGGGATCCCGTGTGCAATGCCTGCGGCCTCTACTACAAGCTACACCACCAGCACTACTGTGGTGGCTCCGCTCA
GCTCATGAGGGCACAGAGCATGGCCTCCAGAGGAGGGGTGGTGTCCTTCTCCTCTTGTAG
Gli2
wildype = AB007295
Deletion in exon 5; deletion of 51 nucleotides
gli2 asv1
AGTGAGTCGGCCGTCAGCAGCACCGTCAACCCTGTCGCCATTCACAAGCGCAGCAAGGTCAAGACCGAGCCTGAG
GGCCTGCGGCCGGCCTCCCCTCTGGCGCTGACGCAGGAGCAGCTGGCTGACCTCAAGGAAGATCTGGACAGGGAT
GACTGTAAGCAGGAGGCTGAGGTGGTCATCTATGAGACCAACTGCCACTGGGAAGACTGC
GLRA2
wildype = AY437083
Alternative exon 3
glra2 asv1
CGGCTTTCTGCAAAGACCATGACTCCAGGTCTGGAAAACAACCTTCACAGACCCTATCTCCTTCAGATTTCTTGG
ACAAGTTAATGGGAAGGACATCAGGATATGATGCAAGAATCAGGCCAAATTTTAAAGGGCCTCCTGTAAATGTTA
CCTGCAACATATTTATCAACAGCTTTGGGTCAATAGCAGAAACTACAATGGACTACCGAGTGAATATTTTTCTGA
GACAACAGTGGAATGATTCACGGCTGGCGTACAGTGAGTACCCAGATGACTCCCTGGACTTGGACCCATCCATGC
TAGACTCCATTTGGAAACCAGATTTGTTCTTTGCCAATGAGAAGGGTGCC
GTF2F1
wildype = X64037
Deletion in exon 5, cryptic splicings in exons 4 and 6; deletion of 396
nucleotides
gtf2f1 asv1
GCTTGAGCAACAAGAAAATCTACCAGGAGGAGGAGAAGGAGAAACGTGGCCGCAGGAAGGCGAGCGAGCTGCGCA
TCCACGACCTGGAGGACGACCTGGAGATGTCGTCCGATGCCAGTGATGCCAGTGGTGAGGAGGGG
GTF2F1
wildype = X64037
general transcription factor IIF, polypeptide 1, 74 kDa
Intron retained between exons 10 and 11; insertion of 79 nucleotides
gtf2f1 asv2
CCCGCAGGAGAAGAAGCGCAGGAAAGACAGCAGCGAGGAGTCGGACAGCTCAGAGGAGAGCGACATTGACAGCGA
GGCCTCCTCAGCCCTCTTCATGGCGGTAAGGCCCAGCCCGGTGGCGGGGGAGGCCTGGGCGTCTGTTTGCAGACT
CACCCAGCTCCCAGCCCTGACCTCTGCAGAAGAAGAAGACGCCACCCAAGAGAGAGCGGAAGCCGTCGGGAGGGA
GCTCAAGGGGCAACAGCCGCCCAGGCACGCCCAGCGCAGAGGGTGGCAGCACCTC
ZNF147
wildype = BC042541
Exon 6 deleted; deletion of 27 nucleotides
znf147 asv1
GGGCGGCTCCAGGAGCTCACCCCCAGTTCAGGTGACCCTGGAGAGCATGACCCAGCGTCCACACACAAATCCACA
CGCCCTGTGAAGAAGGTCTCCACCCCTGTCCCTGCCTTACCCAGCAAGCTTCCCACGTTTGGAGCCCCGGAACAG
TTAGTGGATTTAAAACAAGCTGGCTTGGAGGCTGCAGCCAAAGCCACCAG
Her
wildype = M94166
Alternative exon 7 used
her asv1
AAAACTTTCTGTGTGAATGGAGGGGAGTGCTTCATGGTGAAAGACCTTTCAAACCCCTCGAGATACTTGTGCAAG
TGCCAACCTAACTTCACTGGAGACAGATGTACTGAGAATGTGCCCATGAAAGTCCAAAACCAAGAAAAGGCGGAG
GAGCTGTACCAGAAGAGAGTGCTGACCATAACCGGCATCTGCATCGCCCTCCTTGTGGTCGGCATCATGTGTGTG
GTGGCCTACTGCAAAACCAAGAAACAGCGGAAAAAGCTGCATGACCGTCTTCGGC
MAG
wildype = BC053347
Alternative exon after exon 10; insertion of 45 nucleotides
mag asv1
GGGGACAACCCTCCCGTCCTGTTCAGCAGCGACTTCCGCATCTCTGGGGCACCAGAGAAGTACGAGTCCAAAGAG
GTTTCTACCCTGGAATCTCACTGAGTGCCCCAGGAGAGCGAGAGGCGCCTGGGATCTGAGAGGAGGCTGCTGGGC
CTTCGGGGTGAGCCCCCAGAGCTGGACCTGAGCTATTCTCACTCGGACCTGGGGAAACGG
NCAM
wildype = S71824
Exon insertion between exons 6 and 7; insertion of 30 nucleotides
ncam asv1
CCATCACCTGGAGGACTTCTACCCGGAACATCAGCAGCGAAGAAAAGGCTTCGTGGACTCGACCAGAGAAGCAAG
AGACTCTGGATGGGCACATGGTGGTGCGTAGCCATGCCCGTGTGTCGTCGCTGACCCTGAAGAGCATCCAGTACA
CTGATGCCGGAGAGTACATCTGCACCGCCAGCAACACCATCGGCCAGGACTCCCAGTCCA
NMDAR1
wildype = D13515
Exon 19 deleted, deletion in exon 20; deletion of 464 nucleotides
nmdar1 asv1
CGGGATCTTCCTGATTTTCATCGAGATTGCCTACAAGCGGCACAAGGATGCTCGCCGGAAGCAGATGCAGCTGGC
CTTTGCCGCCGTTAACGTGTGGCGGAAGAACCTGCAGCAGTACCATCCCACTGATATCACGGGCCCGCTCAACCT
CTCAGATCCCTCGGTCAGCACCGTGGTGTGAGGCCCCCGGAGGCGCCCACCTGCCCAGTT
TAU
wildype = BC000558
Exon 10 inserted; insertion of 93 nucleotides
tau asv1
GCCGTCTTCCGCCAAGAGCCGCCTGCAGACAGCCCCCGTGCCCATGCCAGACCTGAAGAATGTCAAGTCCAAGAT
CGGCTCCACTGAGAACCTGAAGCACCAGCCGGGAGGCGGGAAGGTGCAGATAATTAATAAGAAGCTGGATCTTAG
CAACGTCCAGTCCAAGTGTGGCTCAAAGGATAATATCAAACACGTCCCGGGAGGCGGCAGTGTGCAAATAGTCTA
CAAACCAGTTGACCTGAGCAAGGTGACCTCCAAGTGTGGCTCATTAGGCAACATCCATCATAAACCAGGAGGTGG
CCAGGTGGAAGTAAAATCTGAGAAGCTTGACTTCAAGGACAGAGTCCAGT
PGR
wildype = X51730
Exon 4 deleted; deletion of 306 nucleotides
pgr asv1
TGACTGCATCGTTGATAAAATCCGCAGAAAAAACTGCCCAGCATGTCGCCTTAGAAAGTGCTGTCAGGCTGGCAT
GGTCCTTGGAGGTTTTCGAAACTTACATATTGATGACCAGATAACTCTCATTCAGTATTCTTGGATGAGCTTAAT
GGTGTTTGGTCTAGGATGGAGATCCTACAAACACGTCAGTGGGCAGATGCTGTATTTTGC
PGR
wildype = X51730
Exons 4 and 6 deleted; deletion of 306 nucleotides + deletion of 131
nucleotides
pgr1 asv2
TGACTGCATCGTTGATAAAATCCGCAGAAAAAACTGCCCAGCATGTCGCCTTAGAAAGTGCTGTCAGGCTGGCAT
GGTCCTTGGAGGTTTTCGAAACTTACATATTGATGACCAGATAACTCTCATTCAGTATTCTTGGATGAGCTTAAT
GGTGTTTGGTCTAGGATGGAGATCCTACAAACACGTCAGTGGGCAGATGCTGTATTTTGCACCTGATCTAATACT
AAATGATTCCTTTGGAAGGGCTACGAAGTCAAACCCAGTTTGAGGAGATGAGGTCAAGCTACATTAGAGAGCTCA
TCAAGGCAATTGGTTTGAGGCAAAAAGGAGTTGTGTCGAGCTCACAGCGT
ER1
wildype = AF258449
Exon 2 inserted; insertion of 191 nucleotides
er1 asv1
GCCGCCGCAGCTGTCGCCTTTCCTGCAGCCCCACGGCCAGCAGGTGCCCTACTACCTGGAGAACGAGCCCAGCGG
CTACACGGTGCGCGAGGCCGGCCCGCCGGCATTCTACAGGCCAAATTCAGATAATCGACGCCAGGGTGGCAGAGA
AAGATTGGCCAGTACCAATGACAAGGGAAGTATGGCTATGGAATCTGCCAAGGAGACTCGCTACTGTGCAGTGTG
CAATGACTATGCTTCAGGCTACCATTATGGAGTCTGGTCCTGTGAGGGCTGCAAGGCCTTCTTCAAGAGAAGTAT
TCAAGGACATAACGACTATATGTGTCCAGCCACCAACCAGTGCACCATTGATAAAAACAGGAGGAAGAGCTGCCA
GGCCTGCCGGCTCCGCAAATGCTACGAAGTGGGAATGATGAAAGG
RNP6
wildype = AJ419867
Alternatively spliced exon 5; insertion of 766 nucleotides
RNP6 asv1
TATGGCCCGATAAAAGTGAAGGATGTAATAGATCGTGGCCCTTCAATTTAGAAGAGATTAAGAAAAATTGGATGG
AGATTACAGACAGTTCACTCCCTTCCCCCTCAACTCTCCCAATCATTAACATCTTCTATAGTGTGTTACATTTGT
TACAATTAATGAACTGATACTGATACTTTATTATTAAATAAAGTTTAGCATTAACATTAGGGTTTACTCCTGTGT
TGTGCGGCTTTGGACAAATGCAGGAGAGCAAGTCCCACCCAGTGTGCTCTGGAGCAGCCGCTGGCCCTAAACCCC
CTGAGCCATACCTCCCCTTCTTCCTCCCCTTGAACCCCCAAGCAACCGCGAATCTCATTCCTGTCTCTTAAGACT
ACCTTTTCCAAATTGTCACGTCGTTGGAATCATACAGTATGTAGCCTCTGCAGACTGGCTTCTTGCACTTAGCAA
TGTATGTTTGCAGTTCCTCCAGTGTCTTTTCATGACTCGACGGCTCATTGGTTTTTGTTGCTGAAAATATTCCAT
TGTTTGGATGTACACTTTATCCCTTCACCTATAACAGCTTGTATTTTCGTGTGCAGTTTTATGATTACTCAAATT
GCACTTGTAGATATATCTTAACAAACACTTCATACAAAATAAGCATAGTATTATTTTATTCACCAAAGTATTGTT
AATTAGCAGAGCTCAATTCTTTGGTGTCAGTTTATCAAATTTACCTTCTAGGTTTTGAGTTTATTATTAAGAACC
TGCGTAGACTTATTTTATTTTTTAATGCATAGGATCTTTTGCCAGAAATGAGGGCATACTGGCCTGACGTAATTC
ACTCGTTTCCCAATCGCAGCCGCTTCTGGAAGCATGAGTGGGAAAAGCATGGGACCTGCGCCGCCCAGGTGGATG
CGCTCAACTCCCAGAAGAAGTACTTTGGCAGAAGCCTGGAACTCTACAGGGAGCTGGACCTCAACAGTGTGCTTC
TAAAA
LIV1
wildype = BC039498
Additional exon after exon 1; insertion of 780 nucleotides
liv1 asv1
CGTGTGGAACCAAACCTGCGCGCGTGGCCGGGCCGTGGGACAACGAGGCCGCGGAGACGAAGGCGCAATGGCGAG
GAAGTTATCTGTAATCTTGATCCTGACCTTTGCCCTCTCTGTCACAAATCCCCTTCATGAACTAAAAGCAGCTGC
TTTCCCCCAGACCACTGAGAAAATTAGTCCGAATTGGGAATCTGGCATTAATGTTGACTTGGCAATTTCCACACG
GCAATATCATCTACAACAGCTTTTCTACCGCTATGGAGAAAATAATTCTTTGTCAGTTGAAGGGTTCAGAAAATT
ACTTCAAAATATAGGCATAGATAAGATTAAAAGAATCCATATACACCATGACCACGACCATCACTCAGACCACGA
GCATCACTCAGACCATGAGCGTCACTCAGACCATGAGCATCACTCAGACCACGAGCATCACTCTGACCATAATCA
TGCTGCTTCTGGTAAAAATAAGCGAAAAGCTCTTTGCCCAGACCATGACTCAGATAGTTCAGGTAAAGATCCTAG
AAACAGCCAGGGGAAAGGAGCTCACCGACCAGAACATGCCAGTGGTAGAAGGAATGTCAAGGACAGTGTTAGTGC
TAGTGAAGTGACCTCAACTGTGTACAACACTGTCTCTGAAGGAACTCACTTTCTAGAGACAATAGAGACTCCAAG
ACCTGGAAAACTCTTCCCCAAAGATGTAAGCAGCTCCACTCCACCCAGTGTCACATCAAAGAGCCGGGTGAGCCG
GCTGGCTGGTAGGAAAACAAATGAATCTGTGAGTGAGCCCCGAAAAGGCTTTATGTATTCCAGAAACACAAATGA
AAATCCTCAGGAGTGTTTCAATGCATCAAAGCTACTGACATCTCATGGCATGGGCATCCAGGTTCCGCTGAATGC
AACAGAGTTC
SHMT1
wildype = BC038598
Additional exon in 5′ UTR after exon 1; insertion of 140 nucleotides,
splicing does not change the protein composition.
shmt1 asv1
GCAGGGAGACTTCAAGCGCCAAGCTGACCTTTGGAGGTCAGGACGGACCCAGAATCAGGCAGGAATTTGGCAGGC
CCGCGGCGGCGTAGGACGGAGGCGTCGCTAGGGTCTTGTTCTCTTGGCCAGGCTGGAGTGCTGTGGGAAAATCTG
GGCTCACTGCAGCCTCAACCTCCGGGACTCAAGTGATCATCCTGCCTCAGCCACCCCAGAGTAGCTGAGAATACA
GGCGTGCGCCACCAGGCTCGGGCAGCTTCGAACCAGTGCAATGACGATGCCAGTCAACGGGGCCCACAAGGATGC
TGACCTGTGGTCCTCACATGACAAGATGCTGGCACAACCCCTCAAAGACA
CUX
wildype = M74099
Alternative transcription initiation between exons 20 and 21;
If any protein is produced, then downstream Met is used, and protein is a
N-terminal truncation.
cux asv1
GTAAAAGACAGCTATTTTCAGGCACGGTTTCTCGTGTGCTTTAATTACAGAAAGCACTCCAAAGACCTCCGCCAG
CTGCAGCCCTGCCCCTGAGTCCCCG
LZ16
wildype = AF121775
Additional exons after exons 2 and 3; insertion of 273 nucleotides
(additional exon after exon 2); insertion of 97 nucleotides (additional
exon after exon 3); insertion of 370 nucleotides in total
lz16 asv1
CCCAAGGGTGGGTGCCCTAAAGCACCACAGCAGGAAGAGCTTCCCCTCAGCAGCGACATGGTGGAGAAGCAGACT
GGGAAAAAGATTTTTCCAAAAGAATCGTGATCTCAGTGACATATACGTGGAAGATGGAAATGGAGCCCACGACTC
TGCAGTGCATCCTGATGCCGCGCTGACCTGACGGCTTGTGCGTGTCCCTTTGGCTGCACCAGTGAGCACAGTGGC
AGGCGTGTCAGAGAAAGGGCCCCTTCTGCAGACGGTCTCTCACCATTGCCGACCACGGAATCCCAGAACCGCTGA
GCTGCCTCGGGAAGAACCAGCAGGTGTCTGCATCGTTGAGTGTGTTCTGATCCAAAGGATAAAGATAAAGTTTCT
CTAACCAAGACCCCAAAACTGGAGCGTGGCGATGGCGGGAAGGAGGTGAGGGAGCGAGCCAGCAAGCGGAAGCTG
CCCTTCACCGCGGGCGCCAATGGGGAGCAGAAGGACTCGGACACAGATGCCTCCAGCCCAGTCCCTGTTGTGGTG
CTGCAAGGCTGGTACGCTCCTCGAAGCACCATGGCATGAGATGGAGGTTCCTAGAAGCAAGAAGAAAGAGAAGCA
GGGCCCTGAGCGGAAGAGGATTAAGAAGGAGCCTGTCACCCGGAAGGCCGGGCTGCTGTTTGGCATGGGGCTGTC
TGGAATCCGAGCCGGCTACCCCCTC
PMSCL1
wildype = AJ505989
Exon 9 inserted; insertion of 51 nucleotides
pmsc11 asv1
TGATCAAGCTATCATTCTTGATGGTATAAAAATGGACACTGGAGTAGAAGTCTCTGATATTGGAAGCCAAGAGCT
GGGGTTTCACCATGTTGGCCAGACTGGACTCGAGTTCCTGACCTCAGATGCTCCCATAATACTCTCAGATAGTGA
AGAAGAAGAAATGATCATTTTGGAACCAGACAAGAATCCAAAGAAAATAAGAACACAGAC
ANAC
wildype = AF054187
3 additional alternative exons after exon 1; insertion 2130 nucleotides
anac asv1
CTTTCTGCCGCCATCTTGGTTCCGCGTTCCCTGCACAAAATGCCCGGCGAAGCCACAGAAACCGTCCCTGCTACA
GAGCAGGAGTTGCCGCAGCCCCAGGCTGAGACAGCTGTGCTACCTATGTCTTCAGCCTTGAGTGTCACTGCTGCC
TTAGGGCAGCCTGGACCTACCCTCCCCCCTCCTTGCTCTCCTGCCCCACAACAGTGCCCTCTCTCAGCTGCTAAC
CAGGCTTCCCCATTCCCTTCCCCCTCTACTATTGCCTCGACCCCTTTAGAAGTTCCTTTTCCCCAGTCATCCTCT
GGAACAGCCCTACCTTTGGGAACTGCCCCTGAAGCCCCAACCTTCCTACCAAACCTAATAGGGCCTCCCATCTCC
CCAGCTGCCTTAGCTCTAGCCTCTCCCATGATAGCTCCAACTCTGAAAGGGACCCCTTCCTCTTCAGCTCCCTTA
GCTCTGGTTGCCCTGGCTCCCCACTCAGTTCAGAAGAGTTCTGCTTTTCCACCTAACCTTCTTACTTCACCTCCT
TCAGTGGCTGTAGCTGAGTCAGGGTCAGTGATAACTCTGTCAGCTCCCATTGCTCCCTCAGAACCAAAGACTAAT
CTTAATAAAGTTCCCTCTGAGGTAGTCCCTAATCCAAAAGGCACCCCCAGCCCTCCATGTATAGTCAGTACTGTT
CCTTACCACTGTGTGACTCCCATGGCCTCTATTCAATCTGGAGTGGCCTCCCTTCCTCAGACAACACCCACAACT
ACCCTAGCCATCGCTTCCCCTCAAGTCAAAGATACCACCATTTCCTCAGTTCTGATTTCTCCACAAAACCCAGGA
AGCCTCAGCCTGAAGGGGCCTGTTAGTCCACCTGCTGCCTTATCTCTTTCAACTCAGTCTCTTCCTGTGGTGACC
TCTTCTCAAAAGACTGCGGGTCCCAACACCCCCCCAGATTTTCCCATTTCTCTGGGCTCTCATCTTGCACCTTTA
CATCAGAGTTCTTTTGGTTCTGTCCAACTTTTAGGTCAAACAGGTCCTAGTGCTTTGTCAGACCCCACAGAGAAG
ACCATTTCTGTAGATCATTCTTCCACAGGGGCCTCTTATCCTTCTCAGAGATCTGTAATTCCTCCCCTTCCTTCC
AGAAATGAGGTAGTTCCTGCTACTGTGGCTGCCTTTCCAGTGGTGGCTCCATCTGTTGACAAAGGTCCCTCTACC
ATCTCTAGCATAACCTGCAGCCCTTCTGGCTCCTTAAATGTAGCTACCTCTTCTTCATTATCTCCTACAACCTCT
CTCATTCTCAAAAACTCTCCTAATGCCACTTATCATTATCCTTTAGTGGCCCAAATGCCCGTTTCTTCTGTTGGA
ACCACCCCACTTGTGGTGACTAACCCCTGTACAATTGCTGCAGCACCTACTACTACCTTTGAGGTAGCTACTTGT
GTTTCTCCTCCAATGTCATCAGGTCCCATAAGTAACATAGAACCAACTTCCCCTGCTGCCTTGGTTATGGCACCT
GTGGCTCCCAAAGAGCCTTCTACTCAAGTAGCAACCACTCTGAGGATACCAGTCTCTCCTCCTCTGCCAGACCCT
GAAGACCTCAAAAATCTCTCCAGTTCAGTATTGGTTAAATTTCCAACACAAAAAGACCTCCAAACTGTACCTGCC
TCTCTTGAAGGAGCCCCTTTCTCTCCAGCCCAAGCAGGACTCACCACCAAGAAAGACCCTACTGTATTACCGTTA
GTCCAGGCAGCCCCTAAAAATTCCCCTTCTTTCCAAAGTACATCCTCTTCTCCAGAGATACCTCTTTCTCCTGAA
GCCACCCTAGCAAAGAAAAGCCTTGGGGAGCCTCTCCCTATAGTGGCTGCATTTCCTTTGGAAAGTGCTGACCCT
GCCGGGGTGGCTCCCACAACTGCCAAAGCAGCTGCCTTTGAGAAGGTCCTTCCTAAACCTGAATCAGCATCTGTC
TCTGCAGCACCCACCCCACCAGTCTCTCTGCCTCTTGCTCCCTCCCCAGTTCCCACTCTGCCTCCTAAACAGCAA
TTTCTGCCGTCCTCTCCTGGGCTGGTGTTGGAATCACCCTCTAAACCCCTTGCCCCTGCTGATGAGGATGAGCTG
CCGCCTCTGATTCCCCCGGAACCAATCTCTGGGGGAGTGCCTTTCCAGTCGGTCCTCGTCAACATGCCCACCCCT
AAATCTGCTGGAATCCCTGTCCCAACCCCCTCTGCCAAGCAACCTGTTACGAAGAACAACAAGGGGTCTGGAACA
GAATCTGACAGTGATGAATCAGTACCAGAGCTTGAAGAACAGGATTCCACCCAGGCAACCACACAACAAGCCCAG
CTGGCGGCAGCAGCTGAAATCGATGAAGAACCAGTCAGTAAAGCAAAACAGAGTC
Nm23
wildype = AF487339
Exon 2 deleted; deletion of 219 nucleotides
nm23 asv1
TGCAGCCGGAGTTCAAACCTAAGCAGCTGGAAGGAACCATGGCCAACTGTGAGCGTACCTTCATTGCGATCAAAC
CAGATGGGGTCCAGCGGGGTCTTGTGGGAGAGATTATCAAGCGTTTTGAGCAGAAAGGATTCCGC
SWAP70
wildype = BC000616
Exon 3 deleted; deletion of 177 nucleotides
swap70 asv1
GAAGAGCACTTCAGGGATGATGATGAGGGTCCAGTGTCCAACCAGGGCTACATGCCTTATTTAAACAGGTTCATT
TNGGAAAAGATGAATACCTGCTTAAGAAGCTTACAGAAGCTATGGGAGGAGGNTGGCAGCAAGAACAATTTGAAC
ATTATAAAATCAACTTTGATGACAGTAAAAATGGCCTTTCTGCATGGGAACTTATTGAGC
SCRAP
wildype = AK128030
Exon 23 deleted; deletion of 186 nucleotides
scrap asv1
CAGGGGGAAGCAAACCTCTCACCTTCCAAATCCAGGGCAACAAGCTGACTTTGACTGGTGCCCAGGTGCGCCAGC
TTGCTGTGGGGCAGCCCCGCCCGCTGCAAATGCCACCAACCATGGTGAATAATACAGGCGTGGTGAAGATTGTAG
TGAGACAAGCCCCTCGGGATGGACTGACTCCTGTTCCTCCATTGGCCCCAGCACCCCGGC
THTPA
wildype = BX161435
Deletion of 960 nucleotides
thtpa asv1
TCCGGAACTGCTCCCGGCATTCCTCGCGAGTGTATGGCGTGGGCTCCCTTCCCCCTCTGTGGGTCCCGCGAGGAG
ACTCTCGGGCTTTGAGGTGTGCCTGCACAGGAGACAGCACCAGCCAAGCTGATTGTGTATCTACAGCGTTTCCGG
CCTCAAGACTATCAGCGCCTGCTAGAAGTGAACAGCTCCAGAGAGAGGCCACAGGAGACT
SFRS5
wildype = BC018823
Intron retained between exons 4 and 5; insertion of 285 nucleotides
sfrs5 asv1
CTATTGAACATGCTAGGGCTCGGTCACGAGGTGGAAGAGGTAGAGGACGATACTCTGACCGTTTTAGTAGTCGCA
GACCTCGAAATGATAGACGGTATGTGAAGGGTGGATGGCTGCATTGAACAATTATTGTAGGGGTAGCATTTAAGA
TTCAGGAGTCATTAGCAGTGATGATTTTGGGACCTGCCGTATAATCTGTTCTTCTATTCCCACGTTAGCCAATTG
TTCTTGATGAATCTATATGAGTCATAGAACACAAATCTATTGACGGAAGTCATTAGAATGGCTTGTGATATCTGA
TGGCTTGAACTTGCCCACAGTTGAACACAAGTGCTGTCATTGCATTTCTTCCATTGTGAATACGAATTTTCTTCC
TCAGAAATGCTCCACCTGTAAGAACAGAAAATCGTCTTATAGTTGAGAATTTATCCTCAAGAGTCAGCTGGCAGG
ATCTCAAAGATTTCATGAGACAAGCTGGGG
Capn3
wildype = NM_000070
Exon 15 spliced out; deletion of 18 nucleotides
capn3 asv1
GCGAGTACGTCATCGTGCCCTCCACCTACGAGCCCCACCAGGAGGGGGAATTCATCCTCCGGGTCTTCTCTGAAA
AGAGGAACCTCTCTGAGGAAGTTGAAAATACCATCTCCGTGGATCGGCCAGTGCCCATCATCTTCGTTTCGGACA
GAGCAAACAGCAACAAGGAGCTGGGTGTGGACCAGGAGTCAGAGGAGGGCAAAGGCAAAA
CD74
wildype = BC018726
Additional exon after exon 6; insertion of 192 nucleotides
cd74 asv1
ACTGGAAGGTCTTTGAGAGCTGGATGCACCATTGGCTCCTGTTTGAAATGAGCAGGCACTCCTTGGAGCAAAAGC
CCACTGACGCTCCACCGAAAGTACTNACCAAGTGCCAGGAAGAGGTCAGCCACATCCCTGCTGTCCACCCGGGTT
CATTCAGGCCCAAGTGCGACGAGAACGGCAACTATCTGCCACTCCAGTGCTATGGGAGCATCGGCTACTGCTGGT
GTGTCTTCCCCAACGGCACGGAGGTCCCCAACACCAGAAGCCGCGGGCACCATAACTNCAGTGAGTCACTGGAAC
TGGAGGACCCGTCTTCTGGGCTGGGTGTGACCAAGCAGGATCTGGGCCCAGTCCCCATGT
ITGB4
wildype = X51841
Alternative exon after exon 35; insertion of 159 nucleotides
itgb4 asv1
ACTACAACTCACTGACCCGCTCAGAACACTCACACTCGACCACACTGCCGAGGGACTACTCCACCCTCACCTCCG
TCTCCTCCCACGGCCTCCCTCCCATCTGGGAACACGGGAGGAGCAGGCTTCCGCTGTCCTGGGCCCTGGGGTCCC
GGAGTCGGGCTCAGATGAAAGGGTTCCCCCCTTCCAGGGGCCCACGAGACTCTATAATCCTGGCTGGGAGGCCAG
CAGCGCCCTCCTGGGGCCCAGACTCTCGCCTGACTGCTGGTGTGCCCGACACGCCCACCCGCCTGGTGTTCTCTG
CCCTGGGGCCCACATCTCTCAGAGTGAGCTGGCAGGAGCCGCGGTGCGAG
ITPK1
wildype = BC037305
Additional 2 exons after exon1; insertion of 25 nucleotides
itpk1 asv1
GACCTTTCTGAAAGGGAAGAGAGTTGGCTACTGGCTGAGCGAGAAGAAAATCAAGAAGCTGAATTTCCAGGCCTT
CGCCGAGCTGTGCAGGAAGCGAGGGATGGAGGTTGTGCAGCTGAACCTTAGCCGGCCGATCGAGGAGCAGGGCCC
CCTGGACGTCATCATCCACAAGCTGACTGACGTCATCCTTGAAGCCGACCAGAATGATAG
PEG1/MEST
wildype = D87367
Alternative 5′ exon, not translated
pegmest asv1
AGCACATGCTGGGCTCGGGGGCGATGGGCTTGTGCGCGGACCTGGCGACGCTCTAGCCCCGAGCCGCGTATTCGT
GGCCGGGTCCTCCCTGGGAACAGGGTGAAGGCCGAGAACCTCTGGCCTCAGGAAGCGCATGCGCAACCGGTTCTC
CGAAACATGGAGTCCTGTAGGCAAGGTCTTACCTGAATCAGGATGAGGGAGTGGTGGGTCCAGGTGGGGCTGCTG
GCCGTGCCCCTGCTTGCTGCGTACCTGCACATCCCACCCCCTCAGCTCTCCCCTG
MGC2747
wildype = BC001948
Cryptic splice site used in exon 2. No protein.
MGC2747 asv1
AGAATGTTTTTGACCAGAAAACCGACAACCTTCCCAGAAAGTCCAAGCTCGTGGTGGGTGGAAAAGTGTTCGCCG
AGGGTCTGCTTGGCCACTCAGTGCAGCTGCGATTAACCCTAAAGGCTTTAAGGAACGGGCCACCTGTAACAGAGA
CACCAGCCTTCCTGTATAGACACTAAATTG
SMARCD1
wildype = U66617
Exon 1 different + Exon 5 deleted
SMARCD1 asv1
GAAGATGGCGGCCCGGGCGGGTTTCCAGTCTGTGGCTCCAAGCGGCGGCGCCGGAGCCTCAGGAGGGGCGGGCGC
GGCTGCTGCCTTGGGCCCGGGCGGAACTCCGGGGCCTCCTGTGCGAATGGGCCCGGCTCCGGGTCAAGGGCTGTA
CCGCTCCCCGATGCCCGGAGCGGCCTATCCGAGACCAGGTATGTTGCCAGGCAGCCGAATGACACCTCAGGGACC
TTCCATGGGACCCCCTGGCTATGGGGGGAACCCTTCAGTCCGACCTGGCCTGGCCCAGTCAGGGATGGATCAGTC
CCGCAAGAGACCTGCCCCTCAGCAGATCCAGCAGGTCCAGCAGCAGGCGGTCCAAAATCGAAACCACAATGCAAA
GAAAAAGAAGATGGCTGACAAAATTCTACCTCAAAGGATTCGTGAACTGGTACCAGAATCCCAGGCCTATATGGA
TCTCTTGGCTTTTGAAAGGAAACTGGACCAGACTATCATGAGGAAACGGCTAGATATCCAAGAGGCCTTGAAACG
TCCCATCAAGTCAGCCTTGTCCAAATATGATGCCACTAAACAAAAAGAGGAAGTTCTCTTCCTTTTTTAAGTCCC
TTGGTGATTGAACTGGACAAGACCTGTATGGGCCAGACAACNCATCTGGTAGAATGGCA
CDKN2A
wildype = NM_058195
Cryptic splicing, deletion in exon 2; deletion of 75 nucleotides
cdkn2a asv1
CCTGGACACGCTGGTGGTGCTGCACCGGGCCGGGGCGCGGCTGGACGTGCGCGATGCCTGGGGCCGTCTGCCCGT
GGACCTGGCTGAGGAGCTGGGCCATCGCGATGTCGCACGACATCCCCGATTGAAAGAACCAGAGAGGCTCTGAGA
AACCTCCGGAAACTTAGATCATCAGTCACC
CRK
wildype = BC009837
Cryptic splicing, exon 2 internal splicing deletion 46 bp
crk asv1
GGGCACGAGGCTGCTGTGAAGCTGAAACCGGAGCCGGTCCGCTGGGCGGCGGGCGCCGGGGGCCGGAGGGGCGCG
CGCGGCGGCGGCACCCCAGCGTTTAGGCGCGGAGGCAGCCATGGCGGGCAACTTCGACTCGGAGGAGCGGAGTAG
CTGGTACTGGGGGAGGTTGAGTCGGCAGGA
CTDP1
wildype = BC015010
Cryptic splicing in exon III
ctdp1 asv1
GGACGATCACACCAAGGCACAAGAGGGAGAACAGCCCGTGAGGCCATTTCCCGACCGGGAGGGATTGTGCCCCCA
CAACGACATTAGTCCAGACCGAATGCCGGTTCATTCCCAAAGGCCCCAAGCACTGGACCACAGAGGTACGGATAC
ATACGACTCCAACACGGAGAAGCTCATCAGGACACGGGCGCCGAAGGACCCAAAGACCATCCAGGGATCCGTACC
CCATCCGCCAGGAA
TRIM19 lambda
wildype = AF230411
Exon IV deleted, exon V partly deleted; deletion of 143 bp
trim19 asv1
CTGCAGGACCTCAGCTCTTGCATCACCCAGGGGAAAGATGCAGCTGTATCCAAGAAAGCCAGCCCAGAGGCTGCC
AGCACTCCCAGGGACCCTATTGACGTTGACCTGGATGTCTCCAATACAACGACAGCCCAGAAGAGGAAGTGCAGC
CAGACCCAGTGCCCCAGGAAGGTCATCAAG
TCF3
wildype = M31222
Exons III & IV deleted; deletion of 150 bp
tcf3 asv1
ACCAGCCGCAGAGGATGGCGCCTGTGGGCACAGACAAGGAGGCTCAGTGACCTCCTGGACTTCAGCATGATGTTC
CCGCTGCCTGTCACCAACGGGAAGGGCCGGCCCGCCTCCCTGGCCGGGGCGCAGTTCGGAGGTTCAGGCAAGAGC
GGTGAGCGGGGCGCCTATGCCTCCTTCGGG
Bc16
wildype = U00115
Exon 5 spliced into two exons; deletion of 517 nucleotides
bc16 asv1
GAGTTTCGGGATGTCCGGATGCCTGTGGCCAACCCCTTCCCCAAGGAGCGGGCACTCCCATGTGATAGTGCCAGG
CCAGTCCCTGGTGAGTACAGCCACCCATGGAGCCTGAGAACCTTGACCTCCAGTCCCCAACCAAGCTGAGTGCCA
GCGGGGAGGACTCCACCATCCCACAAGCCA
BAG4
wildype = BC038505
Exon skipping, exon II deleted; deletion of 102 bp
bag4 asv1
GGGGGCGGCCCGGCGGAGACCACCTGGCTGGGAGAAGGCGGAGGAGGCGATGGCTACTATCCCTCGGGAGGCGCC
TGGCCAGAGCCTGGTCGAGCCGGAGGAAGCCACCAGAGTTTGAATTCTTATACAAATGGAGCGTATGGTCCAACA
TACCCCCCAGGCCCTGGGGCAAATACTGCC
CNTN4
wildype = AY090737
Exon 8 skipping
cntn4 asv1
GGAATCTGTATATTGCCAAAGTAGAAAAATCAGATGTTGGGAATTATACCTGTGTGGTTACCAATACCGTGACAA
ACCACAAGGTCCTGGGGCCACCTACACCACTAATATTGAGAAATGATGTCCAGTACCAACTATTATCTGGCGAAG
AGCTGATGGAAAGCCAATAGCAAGGAAAGCCAGAAGACACAAGTCAAATGGAATTCTTGAGATCCCTAATTTTCA
CHL1
wildype = NM_006614
Exon 25 skipping.
chl1 asv1
CATTACAACTCCATCAAAGCCCAGCTGGCACCTCTCAAACCTGAATGCAACTACCAAGTACAAATTCTACTTGAG
GGCTTGCACTTCACAGGGCTGTGGAAAACCGATCACGGAGGAAAGCTCCACCTTAGGAGAAGGGAAATATGCTGG
TTTATATGATGACATCTCCACTCAAGGCTGGTTTATTGGACTGATGTGTGCGATTGCTCTTCTCACACTACTATT
ITGA4
wildype = X16983
Insertion of an additional exon after exon 5.
itga4 asv1
CAATAAAACTCAGTCTTGATTTCTGATTATGTGAAAAAATTTGGAGAAAATTTTGCATCATGTCAAGCTGGAATA
TCCAGTTTTTACACAAAGGATTTAATTGTGATGGGGGCCCCAGGATCATCTTACTGGACTGGCTCTCTTTTTGTC
TACAATATAACTACAAATAAATACAAGGCTTTTTTAGACAAACAAAATCAAGTAAAATTTGGAAGTTATTTAGGA
MCAM
wildype = NM_006500
New splice acceptor in exon 16, extended exon.
mcam asv1
GCTCAGGGAAGCAGGAGATCACGCTGCCCCCGTCTCGTAAGACCGAACTTGTAGTTGAAGTTAAGTCAGATAAGC
TCCCAGAAGAGATGGGCCTCCTGCAGGGCAGCAGCGGTGACAAGAGGGCTCCGGGAGACCAGCCCTGAATGTCCT
CGTGACCCCGGAGCTGTTGGAGACAGGTGTTGAATGCACGGCCTCCAACGACCTGGGCAAAAACACCAGCATCCT
SELL
wildype = NM_000655
Exon 7 skipping
sell asv1
CTGTAGCCATCCCCTGGCCAGCTTCAGCTTTACCTCTGCATGTACCTTCATCTGCTCAGAAGGAACTGAGTTAAT
TGGGAAGAAGAAAACCATTTGTGAATCATCTGGAATCTGGTCAAATCCTAGTCCAATATGTCAAAGCAAGAAATC
CAAGAGAAGTATGAATGACCCATATTAAATCGCCCTTGGTGAAAGAAAATTCTTGGAATACTAAAAATCATGAGA
SRrp35
gene id: 135295
asv1, Exon 2 (107 nt) deleted, replaced with new exon 2 (347 nt) just
downstream in the same intron; net change of +240 nt
agctcctgtggtggtagcagcggtagcgggagacggagcgagtccagcggccgcgggcagacccggagggaacgg
aggaagcggtcatgtctcgctacacgaggccccccaacacctccctgttcatcaggaacgtcgcggacgccacca
gaagatctaaagcagtccacagtagctggcaagcaccccccagtttgaaccaacctgttagctagaatccaagca
taaacccagcaggcgagacaaaaggcacctaaagttcaagcatcaaggagtaaagagggagggtggacacagata
taaagacctggaagaggggaagtctttatcaagcaaaagacaaagccaacaccaggttgagacttcggctttcct
acatttactcagagttccagagtcaaagccaagtctgattttgttggttctgcgtctcttataaagtccatcttg
caagccttaaagagtaaaggtcaaggttcaagatcaagtgacattgagatttgaagatgttcgaggtgctgaaga
tgctctttataacctcaatagaaagtgggtatgtggccgtcagattgaaatacagtttgcacaaggtgatcgcaa
aacaccaggccaaatgaaatcaaaagaacgtcatccttgttctccaagtgatcacaggagatcaagaagccccag
ccaaagaagaactcgaagtagaagttcttcatggggaagaaataggaggcggtcagacagccttaaagagtctcg
acacaggcgattttcttatagcaagtctaaatctcgttccaaatcattaccaaggcggtctacctcagcaaggca
gtcaagaactccaagaaggaattttggctctagaggacggtcaaggtccaagtccttacaaaagaggtccaagtc
aataggaaaatcacagtcaagttcacctcaaaagcagactagctcaggaacaaaatcaagatcacatggaagaca
ttctgactcaatagcaagatccccgtgtaaatctcccaaagggtataccaattctgaaactaaagtacaaacagc
aaagcattctcattttcggtcacattccagatctcgaagttatcgtcataaaaacagttggtgaacagcaacaga
aagagca
SFRS14
gene id: 10147
asv1, Extra 93 nt exon between exons 10 and 11
atgtcccctccaggttaagaaagccgaaccagagccgatgcgagaggaggagaaaatgattcctcctacgaaacc
tgaaattcaggccaaggctccaagtagtctgagtgatgctgtcccccagcgagcagatcacagggtagtgggcac
catcgaccagcttgtgaaacgtgtcatcgaaggcagcctgtctcccaaagagagaactcttctcaaagaggaccc
tgcttactggtttttgtctgatgaaaatagtctggagtataaatattacaagctgaagttggcagaaatgcagcg
gatgagcgagaacttgcgaggagccgaccagaagccgacctcagcagactgtgcagtgagggccatgctgtactc
ccgggctgtccgcaacctcaagaagaaactccttccgtggcagcggcgggggctcctccgtgctcaagggctccg
gggctggaaggcgaggagagcgaccaccgggacccagaccctcctatcctcaggcaccaggctgaaacaccacgg
ccggcaggctccaggcctctcacaggcaaaaccatccctgccagacagaaatgatgctgccaaggactgcccgcc
agacccagttggaccttctcctcaggaccccagcttagaagcctcaggcccatcccccaagccagcaggagtgga
catctctgaagcacctcagacctcttctccctgcccatctgctgacattgacatgaagacaatggagactgcaga
gaaactggctagatttgttgctcaggtgggaccagagatcgaacaattcagcatagaaaacagcaccgataaccc
tgacctgtggtttctacatgaccaaaatagttctgctttcaaattctatcgaaagaaagtgtttgaactatgtcc
atcaatttgtttcacgtcatctccgcacaaccttcacactggtggtggtgacaccacgggttctcaggagagccc
cgtggacctcatggaaggggaagcagagtttgaagacgagccccctccgcgggaggctgagctggagagcccaga
ggtgatgcctgaggaggaggacgaggacgatgaggatgggggagaggaggcccccgctcctggaggggcgggcaa
gtctgagggcagcacccctgccgacggccttcccggcgaggctgccgaggacgacctggctggagcacctgcctt
gtcacaggcctcctcaggtacctgcttccctcggaagaggatcagcagcaagtcattgaaggttggcatgattcc
agctcccaagagagtgtgtctcatccaggagccaaaagtccatgaaccagttcgaattgcctatgacaggcctcg
gggtcgtcccatgtccaaaaagaagaaacccaaggacttggacttcgcccagcagaagctgaccgataagaacct
gggcttccagatgctgcagaagatgggctggaaggagggccatggcctgggctccctcggaaagggcatcaggga
gccggtcagcgtgggaaccccctcggaaggggaagggttgggtgctgacgggcaggagcacaaagaagacacatt
cgatgtgttccgacagaggatgatgcagatgtacagacacaagcgggccaacaaatagatcaaaaccactgatgt
gaaagataagccttgaagcagcaattgcccttaaaacatcatccctgccctggatcggcctggagccagtgccca
attccagggtcacccccgagaggacaacaggcatctggaagtgctctctcgccactctgggtgctttactgtctc
tggcttgtttcca
SFRS14
gene id: 10147
asv2, First: Extra 93 nt exon between exons 10 and 11, Second: intron 9
looks unspliced but clone is incomplete; Results in additional 760 nts
atgtcccctccaggttaagaaagccgaaccagagccgatgcgagaggaggagaaaatgattcctcctacgaaacc
tgaaattcaggccaaggctccaagtagtctgagtgatgctgtcccccagcgagcagatcacagggtagtgggcac
catcgaccagcttgtgaaacgtgtcatcgaaggcagcctgtctcccaaagagagaactcttctcaaagaggaccc
tgcttactggtttttgtctgatgaaaatagtctggagtataaatattacaagctgaagttggcagaaatgcagcg
gatgagcgagaacttgcgaggagccgaccagaagccgacctcagcagactgtgcagtgagggccatgctgtactc
ccgggctgtccgcaacctcaagaagaaactccttccgtggcagcggcgggggctcctccgtgctcaagggctccg
gggctggaaggcgaggagagcgaccaccgggacccagaccctcctatcctcaggcaccaggctgaaacaccacgg
ccggcaggctccaggcctctcacaggcaaaaccatccctgccagacagaaatgatgctgccaaggactgcccgcc
agacccagttggaccttctcctcaggaccccagcttagaagcctcaggcccatcccccaagccagcaggagtgga
catctctgaagcacctcagacctcttctccctgcccatctgctgacattgacatgaagacaatggagactgcaga
gaaactggctagatttgttgctcaggtgggaccagagatcgaacaattcagcatagaaaacagcaccgataaccc
tgacctgtggtttctacatgaccaaaatagttctgctttcaaattctatcgaaagaaagtgtttgaactatgtcc
atcaatttgtttcacgtcatctccgcacaaccttcacactggtggtggtgacaccacgggttctcaggagagccc
cgtggacctcatggaaggggaagcagagtttgaagacgagccccctccgcgggaggctgagctggagagcccaga
ggtgatgcctgaggaggaggacgaggacgatgaggatgggggagaggaggcccccgctcctggaggggcgggcaa
gtctgagggcagcacccctgccgacggccttcccggcgaggctgccgaggacgacctggctggagcacctgcctt
gtcacaggcctcctcaggtacctgcttccctcggaagaggatcagcagcaagtcattgaaggttggcatgattcc
agctcccaagagagtgtgtctcatccaggagccaaaagtccatgaaccagttcgaattgcctatgacaggcctcg
gggtcgtcccatgtccaaaaagaagaaacccaaggacttggacttcgcccagcagaagctgaccgataagaacct
gggcttccagatgctgcagaagatgggctggaaggagggccatggcctgggctccctcggaaagggcatcaggga
gccggtcagcgtgtacgcagcaggcagcctggggtgggagtgggtggggcctcagtccttccacctgcagcctgc
cgcttggctccttcacagccaagatggcttacagctggcagttgatttttgttttttaaacagaaggcatcttca
gatgagaagctgatcatttacatgtgcaggtgtttacagggctcctttctgtcctggtgtagattttttaaccag
cttgttggccctggtcattttggccacatttgtgaccatcataaaagctaagtggtatttctgtgtagtttccgt
ctggaactgctttcccattcccgggaacccatagccgggccagccagggtcccgaacacaggcccaaagtttatt
aaaccccgatcataacctccagcaggcatttcatttaatactgagcttagttcctgctgggtaaggcattccgag
gtaaccagggccctctgggcaccccctcaaaagccagctcttcgagggtgagtactccttgtttctactgtgagt
cgcgtcttgattttccctttctttgatgtctcagtgtgtgtcccaaacacctgcatctcatggactgtttgtgcc
catgcccagttcctggcatgccaggccctgggctcaggtgcacaactgactctctttttcactccctaggggaac
cccctcggaaggggaagggttgggtgctgacgggcaggagcacaaagaagacacattcgatgtgttccgacagag
gatgatgcagatgtacagacacaagcgggccaacaaatagcaaaccgtacttgggcactggctccaggccgatcc
agggcagggatgatgttttaagggcaattgctgcttcaaggcttatctttcacatcagtggttttgatttccagg
gtcacccccgagaggacaacaggcatctggaagtgctctctcgccactctgggtgctttactgtctctggcttgt
ttcca
PRPF8
gene id: 10594
asv1, Intron 31 unspliced, results in 292 nt increase
ctaatgctcagcgatcaggactgaaccagattcccaatcgtagattcaccctctggtggtccccgaccattaatc
gagccaatgtatatgtaggctttcaggtgcagctagacctgacgggtatcttcatgcacggcaagatccccacgc
tgaagatctctctcatccagatcttccgagctcacttgtggcagaagatccatgagagcattgttatggacttat
gtcaggtgtttgaccaggaacttgatgcactggaaattgagacagtacaaaaggagacaatccatccccgaaagt
catataagatgaactcttcctgtgcagatatcctgctctttgcctcctataagtggaatgtctcccggccctcat
tgctggctgactccaagtaagtgcctcaggacccagccctaggcagccaggacactttcgttttcctgttcttct
agccctgcaactttaggaattgtcctgtctgcctttgtttcaaacttggagccagtgctacgcttggagcctgtc
aacacccttagtcagatctgctgattctctggggtcctgctgacctggaacaagttggtggagtgggtgggatgg
ttttgggatttaagtggttctggttctggggacattggttatgcccatggtttcttagaagcttgaaccctcttc
atcctcagggatgtgatggacagcaccaccacccagaaatactggattgacatccagttgcgctggggggactat
gattcccacgacattgagcgctacgcccgggccaagttcctggactacaccaccgacaacatgagtatctaccct
tcgcccacaggtgtactcatcgccattgacctggcctataacttgcacagtgcctatggaaactggttcccaggc
agcaagcctctcatacaacaggccatggccaagatcatgaaggcaaaccctgccctgtatgtgttacgtgaacgg
atccgcaaggggctacagctctattcatctgaacccactgagccttatttgtcttctcagaactatggtgagctc
ttctccaaccagattatctggtttgtggatgacaccaacgtctacagagtgactattcacaagacctttgaaggg
aacttgacaaccaagcccatcaacggagccatcttcatcttcaacccacgcacagggcagctgttcctcaagata
atccacacgtccgtgtgggcgggacagaagcgtttggggcagttggctaagtggaagacagctgaggaggtggcc
gccctgatccgatctctgcctgtggaggagcagcccaagcagatcattgtcaccaggaagggcatgctggaccca
ctggaggtgcacttactggacttccccaatattgtcatcaaaggatcggagctccaactccctttccaggcgtgt
ctcaaggtggaaaaattcggggatctcatccttaaagccactgagccccagatggttctcttcaacctctatgac
gactggctcaagactatttcatcttacacggccttctcccgtctcatcctgattctgcgtgccctacatgtgaac
aacgatcgggcaaaagtgatcctgaagccagacaagactactattacagaaccacaccacatctggcccactctg
actgacgaagaatggatcaaggtcgaggtgcagctcaaggatctgatc
PRPF8
gene id: 10594
asv2, intron 31 unspliced, exon 33 has deletion
ctaatgctcagcgatcaggactgaaccagattcccaatcgtagattcaccctctggtggtccccgaccattaatc
gagccaatgtatatgtaggctttcaggtgcagctagacctgacgggtatcttcatgcacggcaagatccccacgc
tgaagatctctctcatccagatcttccgagctcacttgtggcagaagatccatgagagcattgttatggacttat
gtcaggtgtttgaccaggaacttgatgcactggaaattgagacagtacaaaaggagacaatccatccccgaaagt
catataagatgaactcttcctgtgcagatatcctgctctttgcctcctataagtggaatgtctcccggccctcat
tgctggctgactccaagtaagtgcctcaggacccagccctaggcagccaggacactttcgttttcctgttcttct
agccctgcaactttaggaattgtcctgtctgcctttgtttcaaacttggagccagtgctacgcttggagcctgtc
aacacccttagtcagatctgctgattctctggggtcctgctgacctggaacaagttggtggagtgggtgggatgg
ttttgggatttaagtggttctggttctggggacattggttatgcccatggtttcttagaagcttgaaccctcttc
atcctcagggatgtgatggacagcaccaccacccagaaatactggattgacatccagttgcgctggggggactat
gattcccacgacattgagcgctacgcccgggccaagttcctggactacaccaccgacaacatgagtatctaccct
tcgcccacaggtgtactcatcgccattgacctggcctataacttgcacagtgcctatggaaactggttcccaggc
agcaagcctctcatacaacaggccatggccaagatcatgaaggcaaaccctgccctaactatggtgagctcttct
ccaaccagattatctggtttgtggatgacaccaacgtctacagagtgactattcacaagacctttgaagggaact
tgacaaccaagcccatcaacggagccatcttcatcttcaacccacgcacagggcagctgttcctcaagataatcc
acacgtccgtgtgggcgggacagaagcgtttggggcagttggctaagtggaagacagctgaggaggtggccgccc
tgatccgatctctgcctgtggaggagcagcccaagcagatcattgtcaccaggaagggcatgctggacccactgg
aggtgcacttactggacttccccaatattgtcatcaaaggatcggagctccaactccctttccaggcgtgtctca
aggtggaaaaattcggggatctcatccttaaagccactgagccccagatggttctcttcaacctctatgacgact
ggctcaagactatttcatcttacacggccttctcccgtctcatcctgattctgcgtgccctacatgtgaacaacg
atcgggcaaaagtgatcctgaagccagacaagactactattacagaaccacaccacatctggcccactctgactg
acgaagaatggatcaaggtcgaggtgcagctcaaggatctgatc
SR-A1
gene id: 58506
asv1, 81 nt deletion in exon 6
agtctcgagggaagacagaggagtcgggggaggatcggggcgatggtccgccagacagagaccccacgctttctc
cttctgcctttatcctgcgagccatccagcaggctgtgggaagctccctgcagggggacctgcccaatgataaag
atggctctcggtgtcatggccttcgatggcggcgctgccggagtccacggtcagagccccgttcccaggaatcag
ggggcactgacacggctactgtgttggacatggccacggacagcttcctcgcagggctggtgagtgtcctggatc
ccccggatacctgggttcccagccgcctggacctgcggcctggcgaaagtgaggacatgctggagctggtggctg
aggtccgaatcggggacagagatcccatccctctgcctgtgcccagcctgctgccccgtctcagggcctggagga
cgggcaaaacggtttctccacagtcgaactcctctaggcccacctgtgcccgtcacctcaccttgggcacgggag
acgggggccctgcaccgccccctgcacccccagccccacctgccccccgattcgatatctatgaccccttccacc
c
SR-A1
gene id: 58506
asv2, unspliced intron 3 (323 nt increase)
agtctcgagggaagacagaggagtcgggggaggatcggggcgatggtccgccagacagagaccccacgctttctc
cttctgcctttatcctgcgagccatccagcaggctgtgggaagctccctgcagggggacctgcccaatgataaag
atggctctcggtgtcatggccttcgatggcggcgctgccggagtccacggtcagagccccgttcccaggaatcag
ggggcactgacacggctactgtgagtaagaagagggggctgggggcctggctcacgggtatcagggaggaaggga
tgggggcctgagtctgggggaatggggtttggggacctggactcctggctctgcgatgctgaccaggggcaatgt
tggagagtctgggggcctgatctgtgggcctgagctttgagtgttgatggcagtcaggctataggaattagatcc
tcagttttcttggggatcttagatgtctgggttcctgagaggttagggagtggggaagcaggatttgccagtctt
catgtgaccagggacggcgtagagcctctctggcctcttccaggtgttggacatggccacggacagcttcctcgc
agggctggtgagtgtcctggatcccccggatacctgggttcccagccgcctggacctgcggcctggcgaaggtga
ggacatgctggagctggtggctgaggtccgaatcggggacagagatcccatccctctgcctgtgcccagcctgct
gccccgtctcagggcctggaggacgggcaaaacggtttctccacagtcgaactcctctaggcccacctgtgcccg
tcacctcaccttgggcacgggagacgggggccctgccccaccccctgccccctcctctgcatcctcctccccttc
cccttctccctcatcttcctccccttcccctcccccacccccaccgccccctgcacccccagccccacctgcccc
ccgattcgatatctatgaccccttccaccc
SFRS12
gene id: 140890
asv1, exon 9 missing
ccaaagccctctctttattggctcctgctccaaccatgacaagtctgatgcctggtgcaggattgcttccaatac
cgaccccaaatcctttgactactcttggtgtttcacttagcagtttgggagctataccagcagcagcactagacc
ccaacattgcaacacttggagagataccacagccaccacttatgggaaacgtggatccttccaaaatagatgaaa
ttaggagaacggtttatgttggaaatctgaattcccagacaacgacagctgatcaactacttgaattttttaaac
aagttggagaagtgaagtttgtgcggatggcaggtgatgagactcagccaactcggtttgcttttgtggaatttg
cagaccaaaattctgtaccaagggcccttgcttttaatggagttatgtttggagacaggccactgaaaataaatc
actccaacaatgcaatagtaaaaccccctgagatgacacctcaggctgcagctaaggagttagaagaagtaatga
agcgagtacgagaagctcagtcatttatctcagcagctattgaaccagagtctggaaagagcaatgaaagaaaag
gcggtcgatctcgttcccatactcgctcaaaatccaggtctagctcaaaatcccattctagaaggaaaagatcac
aatcaaaacacaggagtagatcccataatagatcacgttcaagacagaaagacagacgtagatctaagagcccac
ataaaaaacgctctaaatcaagggagagacggaagtcaaggagtcgttcgcattcacgggaaaggcgtaggagga
ggagcaggagttcttccagatcgccaagaacatcaaaaaccataaaaaggaaatcttctagatctccgtccccca
ggagcagaaataagaaggataaaaagagagaaaaagaaagggaccacatcagtgaaagaagagagagagaacgtt
caacgtctatgagaaagagttctaatgatagagatgggaaggagaagttggagaagaacagtacttcacteaaag
agaaagageacaataaagaaccagattcaagtgtgagcaaagaagtagatgacaaggatgcaccaaggactgagg
aaaacaaaatacagcacaatgggaattgtcagctgaatgaagaaaacctctctaccaaaacagaagcagtatagg
accgacaagtgtacctctgcactcaatgctggaatcaaatcc
PRPF4
gene id: 9128
asv1, intron 4 unspliced
aaactaaagcacccgacgacttagttgctccggtcgtgaagaaaccacacatctattatggaagtttggaagaga
aggagagggagcgtctggccaaaggagagtctgggattttggggaaagacggacttaaagcagggatcgaagctg
gaaatattaatataacctctggagaagtgtttgaaattgaagagcatatcagcgagcgacaggcagaagtattgg
ctgagtttgagagaaggaagcgagcccggcagatcaatgtttccacagatgactcagaggtcaaagcttgcctta
gagccttgggggaaccatcacacttttttggagagggtcctgctgaaagaagagaaaggttaagaaatatcctct
cagttgtcggtactgatgccttgaaaaagaccaaaaaggatgatgagaagtctaaaaagtccaaagaagaggtag
aacatgtctttaacttcacagtataaacatgaaggaaatgaggggataggtctctcgttttctgctttcaatggt
ttgttttgctgagatgttgggggaaatgtttttgaaggctctaccattcaagaagagttgctggcagtagttttg
gttcctttgtaagtatgaatggagctaagtgagttttccagtcaggaaagaatcatggcattcctggtataacca
tgtagttacatatcatagaaaaaaattcagtagaaagtcctctgcctgatttcatcctattaccgaatgaattca
ccttccttctgggcagttaaaatggagaaatgacagttataagaggagtagaatgcttcagatttgacctttctg
ctcttaatttgcctttcagtatcagcaaacctggtatcatgaaggaccaaatagcttgaaggtggcaagactatg
gattgctaattattcgttgcccagggcaatgaaacgcttggaagaggcccgactccataaggagattcctgagac
aacaaggacctcccagatgcaagagctgcacaagtctctccggtctttgaataatttttgcagtcagattgggga
tgatcggcctatctcctactgtcactttagtcccaattccaagatgctggccacagcttgttggagtgggctttg
caagctctggtctgttcctgattgcaacctccttcacactcttcgagggcataacacaaatgtaggagcaattgt
attccatcccaaatccactgtctccttggacccaaaagatgtcaacctggcctcttgtgcggctgatggctctgt
gaagctttggagtctcgacagtgatgaaccagtggcagatattgaaggccatacagtgcgtgtggcgcgggtaat
gtggcatccttcaggacgtttcctgggcaccacctgctatgaccgttcatggcgcttatgggatttggaggctca
agaggagatcctgcatcaggaaggccatagcatgggtgtgtatgacattgccttccatcaagatggctctttggc
tggcactgggggactggatgcatttggtcgagtttgggacctacgcacaggacgttgtatcatgttcttagaagg
ccacctgaaagaaatctatggaataaatttctcccccaatggctatcacattgcaaccggcagtggtgacaacac
ctgcaaagtgtgggacctccgacagcggcgttgcgtctacaccatccctgctcatcagaacttagtgactggtgt
caagtttgagcctatccatgggaacttcttgcttactggtgcctatgataacacagccaagatctggacgcaccc
aggctggtccccgctgaagactctggctggccacgaaggcaaagtgatgggcctagatatttcttccgatgggca
gctcatagccacttgctcatatgacaggaccttcaagctgtggatggctgaatagatgacaatgggaaaaggact
tg
PRPF4
gene id: 9128
asv2, intron 11 unspliced
aaactaaagcacccgacgacttagttgctccggtcgtgaagaaaccacacatctattatggaagtttggaagaga
aggagagggagcgtctggccaaaggagagtctgggattttggggaaagacggacttaaagcagggatcgaagctg
gaaatattaatataacctctggagaagtgtttgaaattgaagagcatatcagcgagcgacaggcagaagtattgg
ctgagtttgagagaaggaagcgagcccggcagatcaatgtttccacagatgactcagaggtcaaagcttgcctta
gagccttgggggaacccatcacactttttggagagggtcctgctgaaagaagagaaaggttaagaaatatcctct
cagttgtcggtactgatgccttgaaaaagaccaaaaaggatgatgagaagtctaaaaagtccaaagaagagtatc
agcaaacctggtatcatgaaggaccaaatagcttgaaggtggcaagactatggattgctaattattcgttgccca
gggcaatgaaacgcttggaagaggcccgactccataaggagattcctgagacaacaaggacctcccagatgcaag
agctgcacaagtctctccggtctttgaataatttttgcagtcagattggggatgatcggcctatctcctactgtc
actttagtcccaattccaagatgctggccacagcttgttggagtgggctttgcaagctctggtctgttcctgatt
gcaacctccttcacactcttcgagggcataacacaaatgtaggagcaattgtattccatcccaaatccactgtct
ccttggacccaaaagatgtcaacctggcctcttgtgcggctgatggctctgtgaagctttggagtctcgacagtg
atgaaccagtggcagatattgaaggccatacagtgcgtgtggcgcgggtaatgtggcatccttcaggacgtttcc
tgggcaccacctgctatgaccgttcatggcgcttatgggatttggaggctcaagaggagatcctgcatcaggaag
gccatagcatgggtgtgtatgacattgccttccatcaagatggctctttggctggcactgggtaaggcttctccc
atgtagtcaggggcagttcagtactctcacctcttacctatacctgcttccacagagaactggattcaaagtgtt
catttctaaattattttctcaggggactggatgcatttggtcgagtttgggacctacgcacaggacgttgtatca
tgttcttagaaggccacctgaaagaaatctatggaataaatttctcccccaatggctatcacattgcaaccggca
gtggtgacaacacctgcaaagtgtgggacctccgaaagcggcgttgcgtctacaccatccctgctcatcagaact
tagtgactggtgtcaagtttgagcctatccatgggaacttcttgcttactggtgcctatgataacacagccaaga
tctggacgcacccaggctggtccccgctgaagactctggctggccacgaaggcaaagtgatgggcctagatattt
cttccgatgggcagctcatagccacttgctcatatgacaggaccttcaagctgtggatggctgaatagatgacaa
tgggaaaaggacttg
PRPF31
gene id: 26121
asv1, intron 12 unspliced
gcaccgcatctacgagtatgtggagtcccggatgtccttcatcgcacccaacctgtccatcattatcggggcatc
cacggccgccaagatcatgggtgtggccggcggcctgaccaacctctccaagatgcccgcctgcaacatcatgct
gctcggggcccagcgcaagacgctgtcgggcttctcgtctacctcagtgctgccccacaccggctacatctacca
cagtgacatcgtgcagtccctgccaccggatctgcggcggaaagcggcccggctggtggccgccaagtgcacact
ggcagcccgtgtggacagtttccacgagagcacagaagggaaggtgggctacgaactgaaggatgagatcgagcg
caaattcgacaagtggcaggagccgccgcctgtgaagcaggtgaagccgctgcctgcgcccctggatggacagcg
gaagaagcgaggcggccgcaggtaccgcaagatgaaggagcggctggggctgacggagatccggaagcaggccaa
ccgtatgagcttcggagagatcgaggaggacgcctaccaggaggacctgggattcagcctgggccacctgggcaa
gtcgggcagtgggcgtgtgcggcagacacaggtaaacgaggccaccaaggccaggatctccaagacgctgcaggt
atgggccagacccaggtggggctggggaccgagggacacaaggtggggggagcccagatcgcagcctccctgtcc
tccccacagcggaccctgcagaagcagagcgtcgtatatggcgggaagtccaccatccgcgaccgctcctcgggc
acggcctccagcgtggccttcaccccactccagggcctggagattgtgaacccacaggcggcagagaagaaggtg
gctgaggccaaccagaagtatttctccagcatggctgagttcctcaaggtcaagggcgagaagagtggccttatg
tccacctgaatgactgcgtgtgtccaaggtggcttcccactgaagggacacagaggtccagtccttctgaagggc
taggatcgggttctggcagggagaacctgccctgccactggccccattgctgggactgcccagggaggaggcctt
ggaagagtccggcctggcctcccccaggaccgagatcaccgcccagtatgggctagagcaggttttcatcatgcc
ttgt
PRPF31
gene id: 26121
asv2, introns 10 and 12 unspliced
gcaccgcatctacgagtatgtggagtcccggatgtccttcatcgcacccaacctgtccatcattatcggggcatc
cacggccgccaagatcatgggtgtggccggcggcctgaccaacctctccaagatgcccgcctgcaacatcatgct
gctcggggcccagcgcaagacgctgtcgggcttctcgtctacctcagtgctgccccacaccggctacatctacca
cagtgacatcgtgcagtccctgccaccggatctgcggcggaaagcggcccggctggtggccgccaagtgcacact
ggcagcccgtgtggacagtttccacgagagcacagaagggaaggtgggctacgaactgaaggatgagatcgagcg
caaattcgacaagtggcaggagccgccgcctgtgaagcaggtgaagccgctgcctgcgcccctggatggacagcg
gaagaagcgaggcggccgcaggtgaggggccctgggggtccggtaggcatgggggtcatggaggggagaagccgg
cgtcctcctcccagccgactccctggcgccgcccacccacccgtccccaggtaccgcaagatgaaggagcggctg
gggctgacggagatccggaagcaggccaaccgtatgagcttcggagagatcgaggaggacgcctaccaggaggac
ctgggattcagcctgggccacctgggcaagtcgggcagtgggcgtgtgcggcagacacaggtaaacgaggccacc
aaggccaggatctccaagacgctgcaggtatgggccagacccaggtggggctggggaccgagggacacaaggtgg
ggggagcccagatcgcagcctccctgtcctccccacagcggaccctgcagaagcagagcgtcgtatatggcggga
agtccaccatccgcgaccgctcctcgggcacggcctccagcgtggccttcaccccactccagggcctggagattg
tgaacccacaggcggcagagaagaaggtggctgaggccaaccagaagtatttctccagcatggctgagttcctca
aggtcaagggcgagaagagtggccttatgtccacctgaatgactgcgtgtgtccaaggtggcttcccactgaagg
gacacagaggtccagtccttctgaagggctaggatcgggttctggcagggagaacctgccctgccactggcccca
ttgctgggactgcccagggaggaggccttggaagagtccggcctggcctcccccaggaccgagatcaccgcccag
tatgggctagagcaggttttcatcatgccttgt
SF4
gene id: 57794
asv1, unique exon 5
ccccctaaatctggaaaaatgaacatgaacatccttcaccaggaagagctcatcgctcagaagaaacgggaaatt
gaagccaaaatggaacagaaagccaagcagaatcaggtggccagccctcagcccccacatcctggcgaaatcaca
aatgcacacaactcttcctgcatttccaacaagtttgccaacgatggtagcttcttgcagcagtttctgaagttg
cagaaggcacagaccagcacagacgccccgaccagtgcgcccagcgcccctcccagcacacccacccccagcgct
gggaagaggtccctgctcatcagcaggcggacaggcctggggctggccagcctgccgggccctgtgaagagctac
tcccacgccaagcagctgcccgtggcgcaccgcccgagtgtcttccagtcccctgacgaggacgaggaggaggac
tatgagcagtggctggagatcaaagagagagtgtgcctattgactgtggggtgtgtgagttgaaccccagtactg
acagcctccttaaagtttcacccccagagggagccgagactcggaaagtgatagagaaattggcccgctttgtgg
cagaaggaggccccgagttagaaaaagtagctatggaggactacaaggataacccagcatttgcatttttgcacg
ataagaatagcagggaattcctctactacaggaagaaggtggctgagataagaaaggaagcacagaagtcgcagg
cagcctctcagaaagtitcacccccagaggacgaagaggtcaagaaccttgcagaaaagttggccaggttcatag
cggacgggggtcccgaggtggaaaccattgccctccagaacaaccgtgagaaccaggcattcagctttctgtatg
agcccaatagccaagggtacaagtactaccgacagaagctggaggagttccggaaagccaaggccagctccacag
gcagcttcacagcacctgatcccggcctgaagcgcaagtcccctcctgaggccctgtcagggtccttacccccag
ccaccacctgccccgcctcgtccacgcctgcgcccactatcatccctgctccagct
SFRS1
gene id: 6426
asv1, intron 3 unspliced
caaggacattgaggacgtgttctacaaatacggcgctatccgcgacatcgacctcaagaatcgccgcgggggacc
gcccttcgccttcgttgagttcgaggacccgcgagacgcggaagacgcggtgtatggtcgcgacggctatgatta
cgatgggtaccgtctgcgggtggagtttcctcgaagcggccgtggaacaggccgaggcggcggcgggggtggagg
tggcggagctccccgaggtcgctatggccccccatccaggcggtctgaaaacagagtggttgtctctggactgcc
tccaagtggaagttggcaggatttaaaggatcacatgcgtgaagcaggtgatgtatgttatgctgatgtttaccg
agatggcactggtgtcgtggagtttgtacggaaagaagatatgacctatgcagttcgaaaactggataacactaa
gtttagatctcatgaggtaggttatacacgtattcttttctttgaccagaattggatacagtggtcttaacagtg
gaatttcaaggtaaggattcaggcaaggttgtccaagtaaattgccagatttctggttttagttacattgtattc
attcagcatgtctgaagatagatgaaagcttagatctttcaatggaaagttctgtctatccaatagggagaaact
gcctacatccgggttaaagttgatgggcccagaagtccaagttatggaagatctcgatctcgaagccgtagtcgt
agcagaagccgtagcagaagcaacagcaggagtcgcagttactccccaaggagaagcagaggatcaccacgctat
tctccccgtcatagcagatctcgctctcgtacataagatgattggtgacactttttgtagaacccatgttgtata
cagttttcctttattcagtacaatcttttcattttttaattcaaactgttttgttcagaatgggctaaagtgttg
aattgcattcttgtaatatccccttgctcctaacatctacattcccttcgtgtctttgat
SFRS1
gene id: 6426
asv2, exon 1 extended 5′
caaggacattgaggacgtgttctacaaatacggcgctatccgcgacatcgacctcaagaatcgccgcgggggacc
gcccttcgccttcgttgagttcgaggacccgcggtgaggcggcatggggcttgcagccttgaggaaatagagacg
cggaagacgcggtgtatggtcgcgacggctatgattacgatgggtaccgtctgcgggtggagtttcctcgaagcg
gccgtggaacaggccgaggcggcggcggggggtggaggtggcggagctccccgagtcgctatggccccccatcca
ggcggtctgaaaacagagtggttgtctctggactgcctccaagtggaagttggcaggatttaaaggatcacatgc
gtgaagcaggtgatgtatgttatgctgatgtttaccgagatggcactggtgtcgtggagtttgtacggaaagaag
atatgacctatgcagttcgaaaactggataacactaagtttagatctcatgagggagaaactgcctacatccggg
ttaaagttgatgggcccagaagtccaagttatggaagatctcgatctcgaagccgtagtcgtagcagaagccgta
gcagaagcaacagcaggagtcgcagttactccccaaggagaagcagaggatcaccacgctattctccccgtcata
gcagatctcgctctcgtacataagatgattggtgacactttttgtagaacccatgttgtatacagttttccttta
ttcagtacaatcttttcattttttaattcaaactgttttgttcagaatgggctaaagtgttgaattgcattcttg
taatatccccttgctcctaacatctacattcccttcgtgtctttgat
SRPK1
gene id: 6732
asv1, exon 10 missing
agcaggaagaggagattctgggatctgatgatgatgagcaagaagatcctaatgattattgtaaaggaggttatc
atcttgtgaaaattggagatctattcaatgggagataccatgtgatccgaaagttaggctggggacacttttcaa
cagtatggttatcatgggatattcaggggaagaaatttgtggcaatgaaagtagttaaaagtgctgaacattaca
ctgaaacagcactagatgaaatccggttgctgaagtcagttcgcaattcagaccctaatgatccaaatagagaaa
tggttgttcaactactagatgactttaaaatatcaggagttaatggaacacatatctgcatggtatttgaagttt
tggggcatcatctgctcaagtggatcatcaaatccaattatcaggggcttccactgccttgtgtcaaaaaaatta
ttcagcaagtgttacagggtcttgattatttacataccaagtgccgtatcatccacactgacattaaaccagaga
acatcttattgtcagtgaatgagcagtacattcggaggctggctgcagaagcaacagaatggcagcgatctggag
ctcctccgccttccggatctgcagtcagtactgctccccagcctaaaccaaagagtcaagtaccattggccagga
tcaaacgcttatggaacgtgatacagagggtggtgcagcagaaattaattgcaatggagtgattgaagtcattaa
ttatactcagaacagtaataatgaaacattgagacataaagaggatctacataatgctaatgactgtgatgtcca
aaatttgaatcaggaatctagtttcctaagctcccaaaatggagacagcagcacatct
SFRS3
gene id: 6428
asv1, extra exon between exons 3 and 5
aaatgcatcgtgattcctgtccattggactgtaaggtttatgtaggcaatcttggaaacaatggcaacaagacgg
aattggaacgggcttttggctactatggaccactccgaagtgtgtgggttgctagaaacccacccggctttgctt
ttgttgaatttgaagatccccgagatgcagctgatgcagtccgagagctagatggaagaacactatgtggctgcc
gtgtaagagtggaactgtcgaatggtgaaaaaagaagtagaaatcgtggcccacctccctcttggggtcgtcgcc
ctcgagatgattatcgtaggaggagtcctccacctcgtcgcagagtcaccatcatgtctcttctcaccaccctct
gaatctgcattagccagtcaactagccctttcagcgtcatgtgaccagcgcgccccattcagcttggctggtgtc
gtttcacatgacccaggctggccagtcgtcaggttgcaccgccctttggttcccgagcatgctgttttctctcag
ccttctctccaaccttaaccaaatcggcagcagccacctcgaccgcccacacattcctggccaatcagctcagct
gtttatttaccaaatgtcttcacaacaactacagcagcagccttcggctaacaaaaaagcaggaaaaatccacaa
cacccccttcgccaaccaactaaatccaacgcaacatctggcaaaaccttttcagcaaattcttcctggccgtca
gtccggcagcctcacctcaccatttctagcttgttgaaacccaaaactaatctccaagaaggagaagcttctctc
gcagccggagcaggtccctttctagagataggagaagagagagatcgctgtctcgggagagaaatcacaagccgt
cccgatccttctctaggtctcgtagtcgatctaggtcaaatgaaaggaaatagaagacagtttgcaagagaagtg
gtgtacaggaaattacttcatttgacaggagtatgtacagaaaattcaagttttgtttgagacttcataagcttg
gtgcatttttaagatgttttagctgttcaaatctgtttgtctcttgaaacagtgacacaaaggtgtaattctcta
tggtttgaaatggatcatacgaggc

### Autoantibody Detection Platforms

ELISA methods and array-based protein detection methods are well known to those skilled in the art. Peptides for the detection of autoantibodies specific for tumor-enriched or tumor-specific transcription modulator splice variants may be non-diffusibly bound to an insoluble support having isolated sample receiving areas (e.g., a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. In some cases magnetic beads and the like are included. The particular manner of binding of the composition is not crucial so long as it is compatible with the reagents and overall methods of the invention, maintains the activity of the composition and is nondiffusable. Preferred methods of binding include direct binding to “sticky” or ionic supports, chemical crosslinking, the synthesis of peptide on the surface, etc. Following binding of the peptide, excess unbound material is removed by washing. The sample receiving areas may then be blocked through incubation with bovine serum albumin (BSA), casein or other innocuous protein or other moiety.

### Methods and Compositions for Cancer Subtype Diagnosis and Prognosis

It is a further embodiment of the present invention that the disclosed methods of diagnosing and classifying tumors be used by a practitioner to make a prognosis of a neoplastic condition. Because the developmental stage of any particular cell type is characterized by the expression of a unique set of transcription modulators, assaying the expression of transcription modulator splice variants would allow a practitioner to foretell the course of a particular tumor, and/or monitor the course of an ongoing therapeutic regimen.

### Diagnostic and Prognostic Kits

The present invention also encompasses kits for performing the diagnostic and prognostic methods of the invention. Such kits can be prepared from readily available materials and reagents. For example, such kits can comprise any one or more of the following materials: enzymes, reaction tubes, buffers, detergent, primers, probes, antibodies, and peptides. It is preferred that these test kits contain one or more of the primer sequences provided herein to be used to detect the presence of tumor-specific/enriched transcriptional modulator splice variants. In a preferred embodiment, these test kits allow a practitioner to obtain samples of neoplastic cells in blood, tears, semen, saliva, urine, tissue, serum, stool, sputum, cerebrospinal fluid and supernatant from cell lysate. In another preferred embodiment these test kits include the needed apparatus for performing RNA extraction, RT-PCR, and gel electrophoresis. In another embodiment, autoantibody detection kits comprising autoantibody-detecting peptides are provided. Instructions for performing the assays can also be included in the kits.

### Therapeutics and Methods of Treatment

Also disclosed herein are methods for the treatment of cancer, and bioactive agents useful in these methods. Bioactive agents are agents having biological activity. Specifically, they are chemical entities that are capable of reacting with one or more molecules in a cell or in an organism to produce an effect in that cell or organism.

Cancer-associated splice variants of transcription factors, and of basal transcription factors in particular, are preferred therapeutic targets, owing in part to their role in the coordinated regulation (or perturbation) of gene expression in pathological cell states.

Bioactive agents encompass numerous chemical classes, though typically they are organic molecules, preferably small organic compounds having a molecular weight of more than 100 and less than about 2,500 daltons, more preferably between 100 and 2000, more preferably between about 100 and about 1250, more preferably between about 100 and about 1000, more preferably between about 100 and about 750, more preferably between about 200 and about 500 daltons. Bioactive agents comprise functional groups necessary for structural interaction with proteins, particularly hydrogen bonding, and typically include at least an amine, carbonyl, hydroxyl or carboxyl group, preferably at least two of the functional chemical groups. The bioactive agents often comprise cyclical carbon or heterocyclic structures and/or aromatic or polyaromatic structures substituted with one or more of the above functional groups. Bioactive agents are also found among biomolecules including peptides, saccharides, fatty acids, steroids, purines, pyrimidines, derivatives, structural analogs or combinations thereof. Preferred bioactive agents include peptides, e.g., peptidomimetics. Peptidomimetics can be made as described, e.g., in WO 98/56401.

Bioactive agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the bioactive agents are organic chemical moieties or small molecule chemical compositions, a wide variety of which are available in the literature.

In another preferred embodiment, the bioactive agents are nucleic acids. By “nucleic acid” or oligonucleotide or grammatical equivalents herein means at least two nucleotides covalently linked together. A nucleic acid of the present invention will generally contain phosphodiester bonds, although in some cases, as outlined herein, particularly with respect to antisense nucleic acids or probes, nucleic acid analogs are included that may have alternate backbones, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), all of which are incorporated by reference)). Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleoside & Nucleotide, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars, as well as “locked nucleic acids”, are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

Examples of highly preferred bioactive agents are described below, though this description is in no way to be construed as limiting the set of bioactive agents useful in the present methods.

(i) siRNA

Inhibition of the activity of specific isoforms of transcription modulators, particularly tumor-specific or tumor-enriched splice variants of transcription modulators, may be accomplished using short interfering RNA (siRNA). Many reports have established that the activity of specific genes and isoforms can be inhibited using siRNA. For example, see Bai et al., Nucleic Acids Res., 31:7264-70, 2003; Wall et al., Lancet., 362:1401-3, 2003; Zhang et al., Cell, 115:177-86, 2003; Quinn et al., Cancer Res., 63:6221-8, 2003. siRNA may be designed by routine methods in the art, for example using design software, such as siDirect (see Naito et al., Nucleic Acids Res. 2004 Jul. 1; 32(Web Server issue):W124-9; or SVM RNAi. siRNA based on any given target sequence may also be obtained from a commercial source, such as, for example, DHARMACON.

### (ii) Antisense

Inhibition of the activity of specific isoforms of transcription modulators, particularly tumor-specific or tumor-enriched splice variants of transcription modulators, may be accomplished using antisense oligonucleotides. Numerous reports have established that the activity of specific genes and isoforms can be inhibited using antisense oligonucleotides. For example, see Manion et al., Cancer Biol Ther., 2:S105-14, 2003; Zhang et al., Proc Natl Acad Sci, 100:11636-41, 2003; Kabos et al., J Biol. Chem., 277:8763-6, 2002.

(iii) Intrabodies

The use of intrabodies is known in the art, for example, see Marasco, Curr. Top. Microbiol. Immunol. 260:247-270, 2001; Wirtz et al., Prot. Sci. 8(11):2245-50 (1999); Ohage et al. J. Mol. Biol. 291(5):1129-34 and Ohage et al. J. Biol. Chem. 291(5): 1119-28 (1999). Intrabodies may be used to modulate the activity of transcription modulator splice variants in situ.

### (iv) Decoy Nucleic Acids

Inhibition of the activity of specific isoforms of transcription modulators, particularly tumor-specific or tumor-enriched splice variants of transcription modulators, where the transcription modulators are nucleic acid binding proteins, may be accomplished using “decoy” oligonucleotides that specifically bind to the splice variants and inhibit binding to native targets, including regulatory elements in genomic DNA. Numerous reports have established that the activity of specific genes and isoforms can be inhibited using decoy oligonucleotides. For example, see Cho et al., Proc Natl Acad Sci, 99:15626-31, 2002; Ahn et al., Biochem Biophys Res Commun., 310:1048-53, 2003; Morishita, Curr Drug Targets, 4:2 p before 599, 2003.

### (v) Dominant Negative Isoforms

Inhibition of the activity of specific isoforms of transcription modulators, particularly tumor-specific or tumor-enriched splice variants of transcription modulators, may be accomplished using dominant negative isoforms of the transcription modulators. Because much is known about the structure of transcription modulators and the function of individual domains within transcriptional modulators, the function of splice variants can be predicted, and the suitability of the dominant negative technique for the inhibition of splice variant activity can be gauged. Basically, a dominant negative isoform will be designed to lack at least one molecular activity of a targeted splice variant while maintaining other activities and effectively replacing the splice variant with an isoform that is functionally deficient in at least one respect. For example, where the target splice variant is a transcription factor with an identifiable DNA-binding domain, activation domain, and protein:protein interaction motif, a dominant negative may be engineered to maintain the protein:protein interaction motif, but lack the DNA binding domain. Taking the place of the splice variant, the dominant negative will participate in protein:protein interactions with splice variant partners, but be unable to bind DNA as the splice variant normally would. Such a dominant negative design is reminiscent of the Id family of bHLH transcription factor inhibitors.

### (vi) Mimicking Peptides

Inhibition of the activity of specific isoforms of transcription modulators, particularly tumor-specific or tumor-enriched splice variants of transcription modulators, may be accomplished using cell penetrating peptides (CPP) containing “mimicking peptides”. “Mimicking peptides” mimic the interaction domains of transcription factors, i.e., exhibit the function of the interaction domain and may take the place of a splice variant in this respect, and are transported into cells by the CPP. Such CPP-mimicking peptide conjugates have been shown to effectively modulate the activity of transcription factors. For example, see Krosl et al., Nat. Med., 9:1428-32, 2003; Arnt et al., J Biol. Chem., 15; 277(46):44236-43, 2002; Kanovsky et al., Proc Natl Acad Sci, 98(22):12438-43, 2001.

(vii) Small Molecules

Inhibition of the activity of specific isoforms of transcription modulators, particularly tumor-specific or tumor-enriched splice variants of transcription modulators, may be accomplished using small molecules. A small molecule may interfere with any activity possessed by a transcription modulator splice variant that contributes to its ability to modulate transcription. For example, a small molecule may interfere with the ability of a transcription modulator splice variant to enter the nucleus, or to bind DNA, or to heterodimerize with a DNA-binding partner, or to interact with a corepressor molecule, or to interact with a basal transcription factor. Numerous reports have established that the activity of specific genes and isoforms can be inhibited using small molecules. For example, see Berg et al., Proc Natl Acad Sci, 99:3830-5, 2002; Bykov et al., Nat. Med., 8:282-8, 2002.

In a preferred embodiment of the methods provided herein, a small molecule interacts with an amino acid sequence present in the splice variant which is not present in the wildtype counterpart of the transcription modulator.

Preferably, where the transcription modulator splice variant includes a novel amino acid sequence (with respect to wildtype counterpart), a small molecule interacts with a region of the splice variant including the novel amino acid sequence, or a portion thereof.

Preferably, where the transcription modulator splice variant includes an in-frame deletion of amino acids present in its wildtype counterpart, a small molecule interacts with a region of the splice variant including the site at which the deletion occurs.

(viii) Gene Therapy

Where the expression of splice variant transcription modulators endows a tumor cell with a unique transcriptional activity, particularly a transcription activating activity that is mediated by a responsive element in DNA, such activity may be exploited to selectively express toxic agents in tumor cells. Specifically, a recombinant construct comprising a gene encoding a toxic agent under the control of such a responsive element may be engineered and introduced into cells, where it will be selectively expressed in such tumor cells possessing the unique transcriptional activity. Toxic agents may include toxic proteins, peptides, antisense oligonucleotides, and siRNAs. Toxic proteins and peptides are those that are detrimental to cell survival.

By “inhibiting activity” is meant reducing from the activity level observed in the absence of the bioactive agent, including reducing activity to an undetectable level of activity.

### Pharmaceutical Compositions and Treatment

The bioactive agents, either alone or in combination, may be used in vitro, ex vivo, and in vivo depending on the particular application. In accordance, the present invention provides for administering a pharmaceutical composition comprising a pharmaceutically acceptable carrier and a pharmacologically effective amount of one or more of the bioactive agents. The pharmaceutical composition may be formulated as powders, granules, solutions, suspensions, aerosols, solids, pills, tablets, capsules, gels, topical crèmes, suppositories, transdermal patches (e.g., via transdermal iontophoresis), etc.

As used herein, “pharmaceutically acceptable carrier” comprises any of standard pharmaceutically accepted carriers known to those of ordinary skill in the art in formulating pharmaceutical compositions. Thus, bioactive agents, by themselves, such as being present as pharmaceutically acceptable salts, or as conjugates, or where appropriate, nucleic acid vehicles encoding bioactive peptides, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients. Other types of suitable carriers include liposomes, microparticles, nanoparticles, hydrogels, as is well known in the art.

The formulations may include bactericidal agents, stabilizers, buffers, emulsifiers, preservatives, sweetening agents, lubricants, or the like. If administration is by oral route, the oligopeptides may be protected from degradation by using a suitable enteric coating, or by other suitable protective means, for example internment in a polymer matrix such as microparticles or pH sensitive hydrogels.

Suitable carriers, including excipients and diluents, may be found in, among others, Remington's Pharmaceutical Sciences, Mack Publishing Co., Philadelphia, Pa. (17th ed., 1985) and Handbook of Pharmaceutical Excipients, 3rd Ed, Washington D.C., American Pharmaceutical Association (Kibbe, A. H. ed., 2000); hereby incorporated by reference in their entirety. The pharmaceutical compositions described herein can be made in a manner well known to those skilled in the art (e.g., by means conventional in the art, including, by way of example and not limitation, mixing, dissolving, granulating, levigating, emulsifying, encapsulating, entrapping or lyophilizing processes).

The concentrations of the bioactive agents for use in the methods of treatment described herein will be determined empirically in accordance with conventional procedures for the particular purpose. Generally, for administering the bioactive agents ex vivo or in vivo for therapeutic purposes, the bioactive agents are given at a pharmacologically effective dose. By “pharmacologically effective amount” or “pharmacologically effective dose” is an amount sufficient to produce the desired physiological effect or amount capable of achieving the desired result, particularly for treating the disorder or disease condition, including reducing or eliminating one or more symptoms or manifestations of the disorder or disease.

The effective dose administered to the host will vary depending upon what is being administered, the purpose of the administration, such as prophylaxis or therapy, the state of the host, the manner of administration, the number of administrations, interval between administrations, and the like. These can be determined empirically by those skilled in the art and may be adjusted for the extent of the therapeutic response. Factors to consider in determining an appropriate dose include, but are not limited to, size and weight of the subject, the age and sex of the subject, the severity of the symptom, the stage of the disease, method of delivery of the agent, half-life of the agents, and efficacy of the agents. Stage of the disease to consider includes whether the disease is relapsing or in remission phase, and the progressiveness of the disease. Determining the dosages and times of administration for a therapeutically effective amount are well within the skill of the ordinary person in the art.

For example, an initial effective dose can be estimated initially from cell culture assays. Tumor cell proliferation and/or expression of splice variants of the transcriptional modulators may be used to assay effectiveness of the bioactive agent. A dose can then be formulated in animal models to generate a circulating concentration or tissue concentration, including that of the IC50 (concentration of bioactive reagent to achieve 50% reduction in activity being assayed, e.g., cell proliferation) as determined by the cell culture assays. Useful animal models include, but are not limited to, mouse, rat, guinea pigs, rabbits, pigs, monkeys, and chimpanzees.

In addition, the toxicity and therapeutic efficacy may be determined by cell culture assays and/or experimental animals, typically by determining a LD50 (lethal dose to 50% of the test population) and ED50 (therapeutically effectiveness in 50% of the test population). The dose ratio of toxicity and therapeutic effectiveness is the therapeutic index. Preferred are bioactive agents, individually or in combination, exhibiting high therapeutic indices.

For the purposes of this invention, the methods for administering the bioactive agents are chosen depending on the condition being treated, the form of the bioactive agent, and the pharmaceutical composition. Administration of the bioactive agents can be done in a variety of ways, including, but not limited to, cutaneously, subcutaneously, intravenously, orally, topically, transdermally, intraperitoneally, intramuscularly, and intravesically. For example, microparticle, microsphere, and microencapsulate formulations are useful for oral, intramuscular, or subcutaneous administrations. Liposomes and nanoparticles are additionally suitable for intravenous administrations. Administration of the pharmaceutical compositions may be through a single route or concurrently by several routes. For instance, oral administration can be accompanied by intravenous or parenteral injections.

In one embodiment, the method of administration is by oral delivery, in the form of a powder, tablet, pill, or capsule. Pharmaceutical formulations for oral administration may be made by combining one or more of the bioactive agents with suitable excipients, such as sugars (e.g., lactose, sucrose, mannitol, or sorbitol), cellulose (e.g., starch, methyl cellulose, hydroxymethyl cellulose, carboxymethyl cellulose, etc.), gelatin, glycine, saccharin, magnesium carbonate, calcium carbonate, polymers such as polyethylene glycol or polyvinylpyrrolidone, and the like. The pills, tablets, or capsules may have an enteric coating, which remains intact in the stomach but dissolves in the intestine. Various enteric coating are known in the art, a number of which are commercially available, including, but not limited to, methacrylic acid-methacrylic acid ester copolymers, polymer cellulose ether, cellulose acetate phathalate, polyvinyl acetate phthalate, hydroxypropyl methyl cellulose phthalate, and the like. In another embodiment, oral formulations of the bioactive agents are in prepared in a suitable diluent. Suitable diluents include various liquid forms (e.g., syrups, slurries, suspensions, etc.) in aqueous diluents such as water, saline, phosphate buffered saline, aqueous ethanol, solutions of sugars (e.g., sucrose, mannitol, or sorbitol), glycerol, aqueous suspensions of gelatin, methyl cellulose, hydroxylmethyl cellulose, cyclodextrins, and the like. In some embodiments, lipohilic solvents are used, including oils, for instance, vegetable oils, peanut oil, sesame oil, olive oil, corn oil, safflower oil, soybean oil, etc.; fatty acid esters, such as oleates, triglycerides, etc.; cholesterol derivatives, including cholesterol oleate, cholesterol linoleate, cholesterol myristilate, etc.; liposomes; and the like.

In yet another embodiment, the administration is carried out cutaneously, subcutaneously, intraperitonealy, intramuscularly and/or intravenously. Bioactive agents may be dissolved or suspended in a suitable aqueous medium for administration. Additionally, the pharmaceutical compositions for injection may be prepared in lipophilic solvents, which include, but are not limited to, oils, such as vegetable oils, olive oil, peanut oil, palm oil soybean oil, safflower oil, etc; synthetic fatty acid esters, such as ethyl oleate or triglycerides; cholesterol derivatives, including cholesterol oleate, cholesterol linoleate, cholesterol myristilate, etc.; or liposomes, as described above. The bioactive agents may be prepared directly in the lipophilic solvent or as oil/water emulsions, (see for example, Liu, F. et al., Pharm. Res. 12: 1060-1064 (1995); Prankerd, R. J., J. Parent. Sci. Tech. 44: 139-49 (1990); and U.S. Pat. No. 5,651,991).

The delivery systems also include sustained release or long term delivery methods, which are well known to those skilled in the art. By “sustained release or” “long term release” as used herein is meant that the delivery system administers a pharmaceutically therapeutic amount of bioactive agent for more than a day, preferably more than a week, and in certain instances 30 days to 60 days, or longer. Long term release systems may comprise implantable solids or gels, such as biodegradable polymers (see, e.g., Brown, D. M. et al., Anticancer Drugs, 7:507-513 (1996)); pumps, including peristaltic pumps and fluorocarbon propellant pumps; osmotic and mini-osmotic pumps; and the like.

### Development of a Database

Also contemplated herein is the formation of a database correlating transcription modulator splice variant expression with cancer phenotype and response to treatment. The establishment of such a database provides for the optimization of cancer treatment, whereby a precise molecular cancer diagnosis/prognosis is made by transcription modulator splice variant profiling, and consultation of the database reveals what treatments are likely to benefit the patient, and what treatments are likely to have harmful side effects and/or be ineffective for the patient.

### EXPERIMENTAL

### Identification of Tumor-Specific/Enriched Splice Variants of Transcription Modulators Useful for Diagnosis

A number of public databases holding gene expression data derived from a variety of cancer types are well known. For example, National Center for Biotechnology Information's EST database houses records of expressed sequence tags (ESTs) identified in differential display experiments, including ESTs that are upregulated or specific to a variety of cancer types.

Based on the identification of such EST sequences, a genomic database (such as that at NCBI) was consulted to identify corresponding genes. Those which were determined by inspection, using knowledge held in the art, to be multi-exon genes encoding transcription modulators, and thus having the potential to generate transcription modulator splice variants specific to or enriched in cancer, were identified. Primers directed to the distal 5′ (at start) and distal 3′ (at stop) regions of mRNA based on the wildtype sequence were used in RT-PCR reactions with RNA isolated from a variety of tumor cell types, including primary human tumor cell samples and human tumor cell lines. PCR products differing from the wildtype-derived product were sequenced and determined to be transcription modulator splice variants expressed in tumor cells.

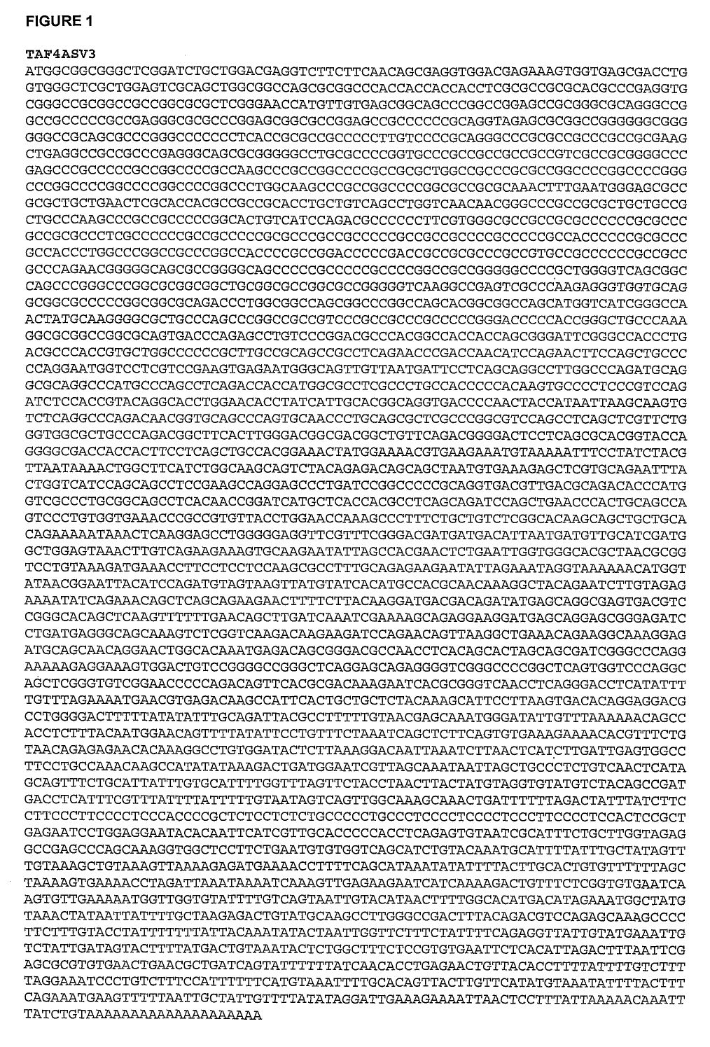

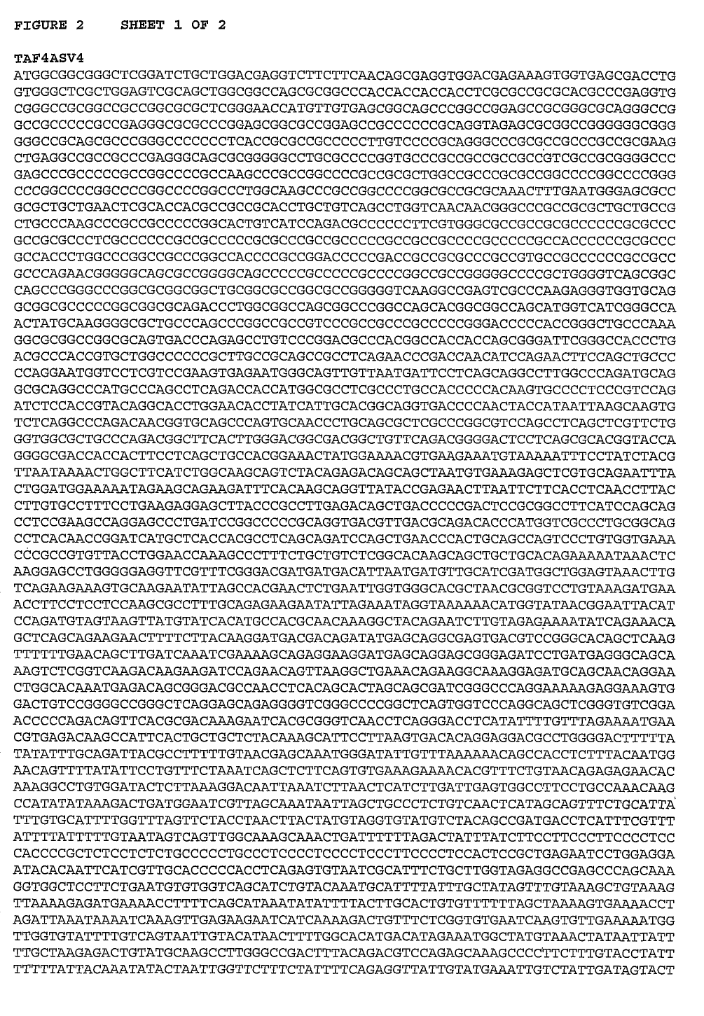

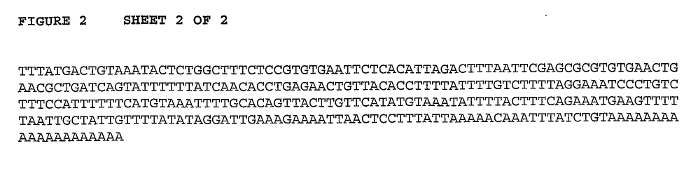

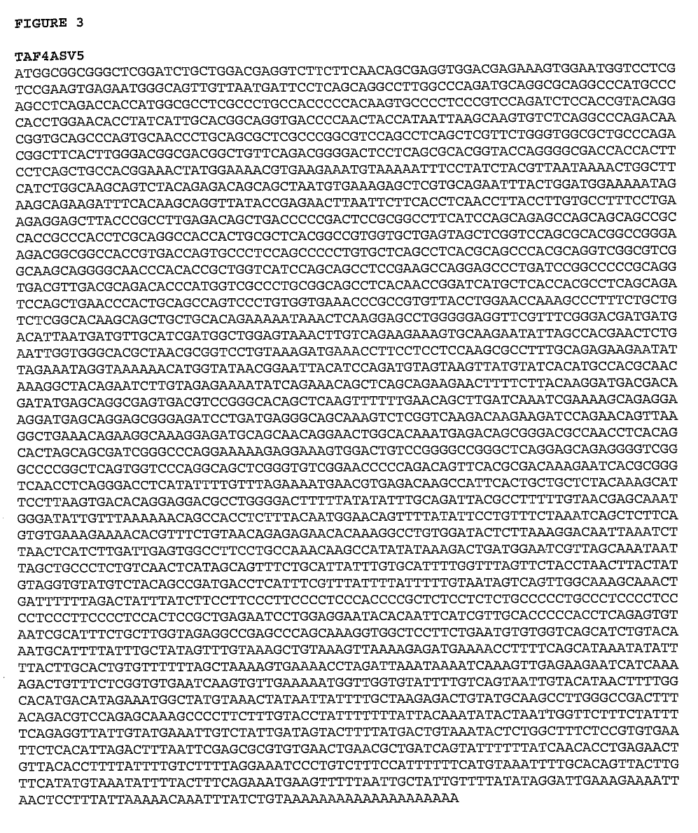

Using this approach, human tumor-specific/enriched splice variants were identified (FIGS. 1-236).

cDNA amplification using RT-PCR is performed as is described in Palm et al., J. Neurosci., 8: 1280-1296 (1998). As with any PCR reaction, triplicate samples were run to ensure the validity of the PCR result. Components and cycling will depend on individual template and primers.

1. To RNA pellet, add 10 μl DEPC—H₂O and 1 μl RNase inhibitor (20 U/μl (Perkin Elmer)).

2. Resuspend the RNA pellet with gentle tapping.

3. Quick spin.

4. Aliquot 5 μl into 2 sterile tubes for (+) and (−) RT reactions.

5. For each batch of samples, prepare additional control tubes as follows, using either high-quality RNA or DEPC-dH₂O in place of the 5 μl sample RNA:

| Control Type | (+) RT | (−) RT |
|---|---|---|
| Positive | High-quality RNA | High-quality RNA |
| Negative | DEPC-dH₂O | DEPC-dH₂O |

6. Prepare sufficient volume of the following +/−RT master reaction mixtures for all reaction tubes:

| (+) RT master reaction mixture | (−) RT master reaction mixture |
|---|---|
| 1.0 μl DEPC-dH₂O | 1.5 μl DEPC-dH₂O |
| 2.0 μl First strand RT buffer | 2.0 μl First strand RT buffer (LT) |
| (Life Technologies) | |
| 1.0 μl dNTP 250 uM (Roche) | 1.0 μl dNTP 250 uM (Roche) |
| 0.5 μl Random hexamer primers | 0.5 μl Random hexamer primers |
| Total volume = 4.5 μl | Total volume = 5.0 μl |

7. Aliquot either 4.5 μl or 5.0 μl of the relevant master mix to the (+) and (−) RT tubes.

8. Incubate at 65° C. for 5 minutes, then at 25° C. for 10 minutes.

9. Add 0.5 μl Superscript II (SSII) reverse transcriptase (Life Technologies to all (+) RT tubes only.

10. Incubate all tubes at 25° C. for 10 minutes, then at 37° C. for 40 minutes.

11. Incubate at 95° C. for 5 minutes to denature the SSII.

12. Quick spin.

13. Aliquot 3 μl of each cDNA sample into a sterile PCR tube.

14. Prepare sufficient volume of PCR master reaction mixture for all reaction tubes and add 7 μl to each tube.

PCR Master Reaction Mixture

1.0 μl PCR Buffer GC-Rich PCR System or the Expand™ Long Distance PCR System kit (Roche)

0.8 μl dNTP 250 μM (Roche)

0.2 μl Forward primer

0.2 μl Reverse primer

(0.2 μl dCTP α-³³P (or α-³²P), in cases when necessary)

0.2 μl polymerase, n U/μl, GC-Rich PCR System or the Expand™ Long Distance PCR System kit (Roche), according to manufacturer's instructions

4.6 (4.4) μl DEPC-dH₂O

Total volume=7 μl

15. PCR Cycling Conditions:

The preferred PCR cycling conditions in general are 35 cycles at 92°, annealing for 1 minute at 56°, and synthesis for one minute at 72°. A specific example follows.

| Cycles | Temp. (° C.) | Time | |
|---|---|---|---|
| 1 | 94 | 2 | min |
| 35-45 | 94 | 30 | seconds |
| | x* | 40 | seconds |
| | 68 or 72 | 150 | seconds |
| 1 | 68 or 72 | 10 | min |

56 is annealing temperature, dependent on the primer used.

16. Store the PCR products at 4° C. or continue to step 5.

17. Pour a 1-2% agarose 6% polyacrylamide sequencing gel (PAGE) while the PCR is cycling.

18. After cycling is complete, add 2.5 μl sample buffer (5×) to samples

19. Denature samples at 95° C. for 3 minutes and place directly on ice.

20. Load 3.5 μl sample on gel and run samples to desired distance.

21. Visualize products on an ethidium bromide treated agarose gel or if PAGE is used, then dry gel and expose to phosphoroimager screen or film.

If necessary, RNA from isolated cell populations is then further characterized for purity by reverse transcriptase-polymerase chain reaction (RT-PCR) with primers specific for a series of established marker genes including: vimentin (stromal cells), cytokeratin 19 (glandular epithelial cells) and CD45 (inflammatory cells/lymphocytes), and other. In addition, more specific markers for NE origin of cells (chromograninA, synaptophysin, 5-hydroxytryptophan receptor, somatostatin receptor or other) can be incorporated.

### RNA Extraction

In a preferred embodiment RNA is extracted from the test and control samples as described in Timmusk et al., Neuron, 10: 475-489 (1993). In brief: To isolate RNA from solid or liquid matrices including blood, stool, sputum, urine, samples are homogenized in 5 ml of Guanidinium lysis buffer (4M Guanidinium isothiocyanate, 25 mM sodium acetate pH 6.0 and 1 mM EDTA pH 8.0; 0.1% DEPC-H₂O; 20% (w/v) N-lauryl sarcosine 10 M; β-mercaptoethanol; 100 mM DTT; RNasin RNase inhibitor (Promega) per 100 μl of the liquid sample, for example. RNA is solubilized by repetitive pipetting. Cell lysates are transferred to a fresh tube and an equal portion (500 μl of the water-saturated acid phenol-chloroform per 100 μl of the liquid sample) is added to the cell lysate. Total RNA is extracted by further ethanol precipitation. In certain applications, liquid matrices (saliva) are first heat-treated (60° C., 15 min) prior to further processing. This is aimed to denature enzymes (salivary) that may affect mRNA stability or interfere with the PCR procedure.

### Preparation of Samples

Blood, ocular discharge, nasal discharge, saliva, feces, CSF, and tissue are collected from healthy and suspected subjects. Peripheral blood mononuclear cells (PBMC) are isolated from 2 ml of whole blood treated with anticoagulant (for example, CPD-A1®, Green Cross Co, Korea) by centrifugation over Ficoll-sodium diatrizoate solution.

Ocular and nasal discharges, saliva, and feces are eluted with 0.5 ml phosphated buffered saline (PBS).

Sputum samples are considered unsatisfactory for evaluation if alveolar lung macrophages are absent or if a marked inflammatory component is present that dilutes the concentration of pulmonary epithelial cells.

Urine often contains very low numbers of tumor cells. In these cases, we recommend concentrating samples of up to 3.5 ml to a final volume of 140 μl, before processing. Concentrated sample of urine are obtained by centrifugation for 10 min at 12,000 rpm. In another application, 30 ml-100 ml of urine samples are spun at 10,000 g, 4° C., 30 min.

Cerebrospinal fluid (CSF) is collected in 0.5 ml samples and processed as non-centrifuged material.

The tumor tissue is obtained through biopsy or surgical resection. For example, tissue samples obtained at resection and biopsies are fixed by perfusion or immersion in neutral buffered formalin (NBF), respectively. A portion of each tumor sample is frozen in liquid nitrogen and the remaining tumor tissue is fixed in NBF, embedded in paraffin; 5-μm sections are cut, and stained with hematoxylin and eosin to identify precursor lesions. Lung lobes obtained from patients undergoing resection were sampled as follows. The normal tissue surrounding the tumor is sampled extending in all directions toward the periphery of the tumor. Approximately eight separate pieces of tissue are embedded in paraffin, sectioned, and stained with hematoxylin and eosin to identify precursor lesions. Lesions are classified based on World Health Organization criteria. Sequential sections from biopsies and lesions identified in resections are cut (5-10 μm), deparaffinized, and stained with toluidine blue to facilitate dissection. A 25-gauge needle attached to a tuberculin syringe is used to remove the lesions under a dissecting microscope. Because of the extensive contamination of some lesions with normal tissue (e.g., SCC, adenoma, alveolar hyperplasia) or the small size of some lesions, <0.001 mm³, it is essential to include normal appearing cells to ensure that enough sample remained to conduct the RT-PCR assay as described below. Since, because the goal of the diagnostic analysis is to determine whether abnormal splice variants are present in these lesions and not to quantitate their levels, the presence of normal tissue-“contaminant” is acceptable. In cases where the lesion is pure, of substantial size (>500 cells), and easily dissected, it is possible to microdissect only the lesion itself.

### Expression of Transcription Modulator Splice Variants in a Variety of Cancer Types

TABLE 3

| | | | EXPRESSION | | | | | | |
|---|---|---|---|---|---|---|---|---|---|
| | | | breast | lung | | | | glioblastoma | |
| Factor | ASV | cDNA | cancer | cancer | melanoma | SCLC1 | SCLC2 | G3 | GBM |
| TAF | | | | | | | | | |
| TAF2 | TAF2 | | P | P | P | P | P | P | P |
| | TAF2 ASV1 | insert 165 nt after ex. 9 | P | N | N | N | N | N | N |
| | TAF2 ASV2 | insert 152 nt after ex. 9 | P | N | N | N | N | N | N |
| TAF4 | TAF4 (S2/AS3) | | N | N | N | N | N | N | N |
| | TAF4 ASV1 | exons 6-9 spliced out | P | P | P | N | N | N | N |
| | TAF4 ASV2 (S2/As2) | exon 7 spliced out | N | N | P | P | P | P | P |
| TAF7L | TAF7L | | P | N | P | N | N | N | N |
| | TAF7L ASV1 | new exon between ex. 8 and 9 | P | N | P | N | P | N | N |
| TAF10 | TAF10 | | P | P | P | P | P | P | P |
| | TAF10 ASV1 | intron seq. after exon 2 | P | P | P | N | N | N | N |
| | TAF10 ASV2 | intron seq after exon 4 | P | P | P | N | N | N | N |
| | TAF10 ASV3 | intron seq. after exon 2 | P | P | P | N | N | N | N |
| | TAF10 ASV4 | intron after exon 2 and exon 4 | N | P | P | N | N | N | N |
| TAF15 | TAF15 (S2/AS2) | | P | P | P | P | P | P | P |
| | TAF15 ASV1 | exon 15 spliced out | N | N | N | P | P | P | P |
| SMARC | | | | | | | | | |
| SMARCA1 | SMARCA1 (S3/AS2) | | P | P | P | P | P | P | P |
| | SMARCA1 ASV1 | exon 13 is spliced out (fragment 219) | N | N | P | P | P | P | P |
| SMARCA2 | SMARCA2 (S6/AS6) | | P | P | P | P | P | P | P |
| | SMARCA2 ASV1 | deletion in ex 29 (fragment 834) | N | N | N | P | P | P | P |
| SMARCA4 | SMARCA4 (S6/AS6) | | P | P | P | P | P | P | N |
| | SMARCA4 ASV1 | exon 27 is out (fragment 950) | P | P | P | P | P | P | P |
| SMARCB1 | SMARCB1 | | P | P | P | P | P | P | P |
| | SMARCB1 ASV1 | Deletion in exon 2 (nt 355-378) | P | P | P | P | P | P | P |
| SMARCC2 | SMARCC2 (S5/AS5) | | P | P | P | P | P | P | P |
| | SMARCC2 ASV1 | nt 3255-3600 spliced in exon 27 | P | P | P | P | P | N | N |
| | SMARCC2 ASV2 | nt 3255-3531 spliced in exon 27 | P | P | P | P | P | N | N |
| | SMARCC2 ASV3 | extra ex. between 17 and 18 (fr. 1050) | N | N | N | N | N | P | P |
| SMARCD3 | SMARCD3 | | N | N | N | N | N | N | N |
| | SMARCD3 ASV1 | New ORF or short trunc (frag. 1400) | P | N | P | N | N | P | P |
| | SMARCD3 ASV2 | ex.s 3, 4, 5 out (frag. 1300) | N | N | N | P | P | N | N |
| NCOA | | | | | | | | | |
| NCOA2 | NCOA2 (S2/AS2) | | P | P | P | P | P | P | P |
| | NCOA2 ASV1 | ex 13 spliced out (fr. 1100) | P | P | P | P | P | P | P |
| NCOA4 | NCOA4 (S1/AS2) | | P | P | P | P | P | P | P |
| | NCOA4 ASV1 | exon 8 out (frag. 900) | P | P | P | P | P | P | P |
| NCOA6 | NCOA6 (S2/AS2) | | P | P | P | P | P | P | P |
| | NCOA6 ASV1 | deletion beginning of ex 8 (fr. 571) | N | N | N | N | N | P | P |
| NCOA7 | NCOA7 (S1/AS1) | | P | P | P | P | P | P | P |
| | NCOA7 ASV1 | exon 3 out (fr. 600) | P | P | P | P | P | P | P |

All references cited herein are expressly incorporated herein in their entirety by reference. All sequences referenced herein by Genbank accession numbers are incorporated herein in their entirety by reference.

We claim:

1. A method for diagnosing cancer, comprising determining the expression of at least one splice variant of each of a plurality of basal transcription factors, wherein expression of each of said basal transcription factor splice variants is distinguished from expression of its wildtype isoform, and wherein the expression pattern of said basal transcription factor splice variants is indicative of cancer.

2. The method according to claim 1, further comprising determining the expression of a plurality of splice variants of at least one of said plurality of basal transcription factors, wherein expression of each of the basal transcription factor splice variants is distinguished from the expression of its counterpart wildtype isoform, and wherein the expression pattern of said basal transcription factor splice variants is indicative of cancer.

3. A method for diagnosing cancer, comprising determining the expression of a plurality of splice variants of at least one basal transcription factor, wherein expression of each of said basal transcription factor splice variants is distinguished from expression of its counterpart wildtype isoform, and wherein the expression pattern of said basal transcription factor splice variants is indicative of cancer.

4. The method according to claim 3, further comprising determining the expression of a plurality of splice variants of a plurality of basal transcription factors, wherein expression of each of said splice variants is distinguished from expression of the wildtype isoform of the corresponding transcription modulator, and wherein the expression pattern of said splice variants is indicative of cancer.

5. The method according to any one of claims 1 to 4, wherein the expression pattern of said basal transcription factor splice variants is indicative of at least one cancer selected from the group consisting of lung cancer, gastrointestinal cancer, breast cancer, prostate cancer, skin cancer, sarcoma, endocrine cancer, neural cancer, bladder cancer, cervical cancer, renal cancer, and hematopoietic cancer.

6. The method according to any one of claims 1 to 4, wherein said basal transcription factor splice variants are derived from the group of gene families consisting of TAF, SMARC, HDAC, MED12, NCOA, GTF, THRAP, HMG, OGDL, BRF, and BAF.

7. The method according to any one of claims 1 to 4, wherein the expression pattern of said splice variants is determined simultaneously.

8. The method according to any one of claims 1 to 4, wherein said determining the expression of at least one splice variant comprises determining the expression of at least one mRNA encoding said at least one splice variant.

9. The method according to claim 8, wherein said determining the expression of at least one mRNA comprises the use of a nucleic acid array.

10. The method according to claim 8, wherein said determining the expression of at least one mRNA comprises the use of RT-PCR.

11. The method according to any one of claims 1 to 4, wherein said determining the expression of at least one splice variant comprises determining the presence of an autoantibody in a sample, which autoantibody specifically binds to said at least one splice variant.

12. The method according to claim 11, wherein said determining the presence of an autoantibody comprises the use of a peptide that specifically binds to said autoantibody.

13. The method according to claim 12, further comprising the use of a peptide array.

14. The method according to any one of claims 1 to 4, further comprising determining the expression of at least one splice variant of at least one non-basal transcription factor, wherein expression of each of the non-basal transcription factor splice variants is distinguished from the expression of its counterpart wildtype isoform, and wherein the expression of said non-basal transcription factor splice variants is indicative of cancer.

15. The method according to any one of claims 1 to 4, further comprising determining the expression of at least one splice variant of at least one non-transcription modulator, wherein expression of each of the non-transcription-modulator splice variants is distinguished from the expression of its counterpart wildtype isoform, and wherein the expression of said non-transcription modulator splice variants is indicative of cancer.

16. A method for the treatment of cancer, comprising administering to said patient a bioactive agent capable of inhibiting the activity of basal transcription factor splice variant; wherein expression of said basal transcription factor splice variant is distinguished from expression of its counterpart wildtype isoform, and wherein the expression of said basal transcription factor splice variant is indicative of cancer.

17. The method according to claim 16, wherein said basal transcription factor splice variant is derived from the group of gene families consisting of TAF, SMARC, HDAC, MED12, NCOA, GTF, THRAP, HMG, OGDL, BRF, and BAF.

18. The method according to claim 16 or 17, wherein said bioactive agent is a small interfering RNA.

19. The method according to claim 16 or 17, wherein said bioactive agent is an antisense nucleic acid.

20. The method according to claim 16 or 17, wherein said bioactive agent is a decoy oligonucleotide which is capable of binding to said at least one splice variant of a basal transcription factor.

21. The method according to claim 16 or 17, wherein said bioactive active agent directly targets one or more of said basal transcription factor splice variants and is selective for said one or more basal transcription factor splice variants over their counterpart wildtype isoforms.

22. A nucleic acid encoding a basal transcription factor splice variant, comprising a nucleotide sequence selected from the group consisting of SEQ ID No: yy to zz.

23. A basal transcription factor splice variant, comprising an amino acid sequence encoded by a nucleic acid according to claim 22.

24. A nucleic acid encoding a partial amino acid sequence of a basal transcription factor splice variant, comprising a nucleotide sequence selected from the group consisting of SEQ ID No: 1 to xx.

25. An antibody that specifically binds to a partial amino acid sequence of a basal transcription factor according to claim 24, wherein said antibody does not specifically bind to the wildtype isoform of the counterpart basal transcription factor.

26. A diagnostic array for detecting cancer, comprising at least a first peptide capable of binding with an autoantibody that recognizes a splice variant of a first basal transcription factor and a second peptide capable of binding with an autoantibody that recognizes a splice variant of a second basal transcription factor; wherein said first and second peptides do not specifically bind to autoantibodies that recognize the wildtype isoforms of said first and second basal transcription factors.

27. A diagnostic array for detecting cancer, comprising at least a first peptide capable of binding with an autoantibody that recognizes a first splice variant of a basal transcription factor and a second peptide capable of binding with an autoantibody that recognizes a second splice variant of said basal transcription factor; wherein said first and second peptides do not specifically bind to autoantibodies that recognize the wildtype isoform of said basal transcription factor.

28. The array according to claim 26 or 27, wherein said peptides are non-diffusably bound to a solid support.
